(19) United States
(12) Patent Application Publication
(10) Pub. No.: US 2023/0072421 A1
(43) Pub. Date: Mar. 9, 2023

(54) TARGETED RADIOPHARMACEUTICALS FOR THE DIAGNOSIS AND TREATMENT OF PROSTATE CANCER

(71) Applicants: Bayer AS, Oslo (NO); Bayer Aktiengesellschaft, Leverkusen (DE)

(72) Inventors: Niels BÖHNKE, Berlin (DE); Sabine ZITZMANN-KOLBE, Berlin (DE); Stefanie HAMMER, Berlin (DE); Sven WITTROCK, Berlin (DE); Donald BIERER, Haan (DE); Thorsten POETHKO, Schwelm (DE); Hans BRIEM, Bremen (DE); Holger Magnus STEUBER, Berlin (DE); Martina SCHÄFER, Berlin (DE); Robin Michael MEIER, Wain (DE); Arif CELIK, Berlin (DE); Cornelia PREUSSE, Berlin (DE); Antje ROTTMANN, Berlin (DE); Nicolas WERBECK, Berlin (DE); Alexander KRISTIAN, Oslo (NO); Bård INDREVOLL, Oslo (NO); Alan CUTHBERTSON, Oslo (NO); Alex PAPPLE, Aurskog (NO)

(73) Assignees: Bayer AS, Oslo (NO); Bayer Aktiengesellschaft, Leverkusen (DE)

(21) Appl. No.: 17/629,258

(22) PCT Filed: Jul. 24, 2020
(86) PCT No.: PCT/EP2020/070922

§ 371 Date: Jan. 21, 2022

(30) Foreign Application Priority Data
Jul. 25, 2019 (EP) 19188231.5

(51) Int. Cl. A61K 51/10; A61P 35/00; A61K 51/04; A61K 51/06; A61K 9/00; A61K 45/06

(52) U.S. Cl. CPC A61K 51/1096; A61P 35/00; A61K 51/0478; C07B 2200/05; A61K 51/0497; A61K 9/0019; A61K 45/06; A61K 51/065

(57) ABSTRACT

A compound of general formula (I): wherein: n is 1, 2 or 3; R1, R2, R3 and R4, independently represent OH or Q; and 20 Q represents a tissue-targeting moeity selected from the group consisting of or a stereoisomer, a hydrate, a solvate, or a salt thereof, or a mixture of same, methods of preparing said compounds, intermediate compounds useful for preparing said compounds, pharmaceutical compositions and combinations comprising said compounds and the use of said 25 compounds for manufacturing pharmaceutical compositions for the treatment or prophylaxis of diseases, in particular of soft tissue diseases, as a sole agent or in combination with other active ingredients.

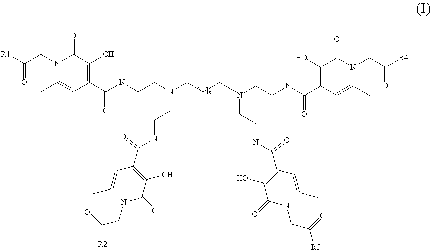

The present invention relates to chelators of general formula (I) and targeted radiopharmaceuticals prepared thereform as described and defined herein, methods of preparing said conjugates, intermediate compounds useful for preparing said compounds, pharmaceutical compositions and combinations comprising said compounds, and the use of said compounds for manufacturing pharmaceutical compositions for the imaging, treatment or prophylaxis of diseases, in particular prostate cancer, as a sole agent or in combination with other active ingredients.

### BACKGROUND

Specific cell killing can be essential for the successful treatment of a variety of diseases in mammalian subjects. Typical examples of this are in the treatment of malignant diseases such as sarcomas and carcinomas. However the selective elimination of certain cell types can also play a key role in the treatment of other diseases, especially hyperplastic and neoplastic diseases.

The most common methods of selective treatment are currently surgery, chemotherapy and external beam irradiation. Targeted radionuclide therapy is, however, a promising and developing area with the potential to deliver highly cytotoxic radiation specifically to cell types associated with disease. The most common forms of radiopharmaceuticals currently authorised for use in humans employ beta-emitting and/or gamma-emitting radionuclides. There has, however, been some interest in the use of alpha-emitting radionuclides in therapy because of their potential for more specific cell killing.

The radiation range of typical alpha emitters in physiological surroundings is generally less than 100 micrometers, the equivalent of only a few cell diameters. This makes these sources well suited for the treatment of tumours, including micrometastases, because they have the range to reach neighbouring cells within a tumour but if they are well targeted then little of the radiated energy will pass beyond the target cells. Thus, not every cell need be targeted but damage to surrounding healthy tissue may be minimised (see Feinendegen et al., Radiat Res 148:195-201 (1997)). In contrast, a beta particle has a range of 1 mm or more in water (see Wilbur, Antibody Immunocon Radiopharm 4: 85-96 (1991)).

The energy of alpha-particle radiation is high in comparison with that carried by beta particles, gamma rays and X-rays, typically being 5-8 MeV, or 5 to 10 times that of a beta particle and 20 or more times the energy of a gamma ray. Thus, this deposition of a large amount of energy over a very short distance gives α-radiation an exceptionally high linear energy transfer (LET), high relative biological efficacy (RBE) and low oxygen enhancement ratio (OER) compared to gamma and beta radiation (see Hall, “Radiobiology for the radiologist”, Fifth edition, Lippincott Williams & Wilkins, Philadelphia Pa., USA, 2000). This explains the exceptional cytotoxicity of alpha emitting radionuclides and also imposes stringent demands on the biological targeting of such isotopes and upon the level of control and study of alpha emitting radionuclide distribution which is necessary in order to avoid unacceptable side effects.

Table 1 below shows the physical decay properties of the alpha emitters so far broadly proposed in the literature as possibly having therapeutic efficacy.

TABLE 1

| Candidate nuclide | T_{1/2}* | Clinically tested for |
|---|---|---|
| ²²⁵Ac | 10.0 days | leukaemia |
| ²¹¹At | 7.2 hours | glioblastoma |
| ²¹³Bi | 46 minutes | leukaemia |
| ²²³Ra | 11.4 days | skeletal metastases |
| ²²⁴Ra | 3.66 days | ankylosing spondylitis |

*Half life

So far, with regards to the application in radioimmunotherapy the main attention has been focused on ²¹¹At, ²¹³Bi and ²²⁵Ac and these three nuclides have been explored in clinical immunotherapy trials.

Several of the radionuclides which have been proposed are short-lived, i.e. have half-lives of less than 12 hours. Such a short half-life makes it difficult to produce and distribute radiopharmaceuticals based upon these radionuclides in a commercial manner. Administration of a short-lived nuclide also increases the proportion of the radiation dose which will be emitted in the body before the target site is reached.

The recoil energy from alpha-emission will in many cases cause the release of daughter nuclides from the position of decay of the parent. This recoil energy is sufficient to break many daughter nuclei out from the chemical environment which may have held the parent, e.g. where the parent was complexed by a ligand such as a chelating agent. This will occur even where the daughter is chemically compatible with, i.e. complexable by, the same ligand. Equally, where the daughter nuclide is a gas, particularly a noble gas such as radon, or is chemically incompatible with the ligand, this release effect will be even greater. When daughter nuclides have half-lives of more than a few seconds, they can diffuse away into the blood system, unrestrained by the complexant which held the parent. These free radioactive daughters can then cause undesired systemic toxicity.

The use of Thorium-227 (T_{1/2}=18.7 days) under conditions where control of the ²²³Ra daughter isotope is maintained was proposed a few years ago (see WO 01/60417 and WO 02/05859).

This was in situations where a carrier system is used which allows the daughter nuclides to be retained by a closed environment. In one case, the radionuclide is disposed within a liposome and the substantial size of the liposome (as compared to recoil distance) helps retain daughter nuclides within the liposome. In the second case, bone-seeking complexes of the radionuclide are used which incorporate into the bone matrix and therefore restrict release of the daughter nuclides. These are potentially highly advantageous methods, but the administration of liposomes is not desirable in some circumstances and there are many diseases of soft tissue in which the radionuclides cannot be surrounded by a mineralised matrix so as to retain the daughter isotopes.

More recently, it was established that the toxicity of the ²²³Ra daughter nuclei released upon decay of ²²⁷Th could be tolerated in the mammalian body to a much greater extent than would be predicted from prior tests on comparable nuclei. In the absence of the specific means of retaining the radium daughters of thorium-227 discussed above, the publicly available information regarding radium toxicity made it clear that it was not possible to use thorium-227 as a therapeutic agent since the dosages required to achieve a therapeutic effect from thorium-227 decay would result in a highly toxic and possibly lethal dosage of radiation from the decay of the radium daughters, i.e. there is no therapeutic window.

WO 04/091668 describes the unexpected finding that a therapeutic treatment window does exist in which a therapeutically effective amount of a targeted thorium-227 radionuclide can be administered to a subject (typically a mammal) without generating an amount of radium-223 sufficient to cause unacceptable myelotoxicity. This can therefore be used for treatment and prophylaxis of all types of diseases at both bony and soft-tissue sites.

In view of the above developments, it is now possible to employ alpha-emitting thorium-227 nuclei in endoradionuclide therapy without lethal myelotoxicity resulting from the generated ²²³Ra. Nonetheless, the therapeutic window remains relatively narrow and it is in all cases desirable to administer no more alpha-emitting radioisotope to a subject than absolutely necessary. Useful exploitation of this new therapeutic window would therefore be greatly enhanced if the alpha-emitting thorium-227 nuclei could be complexed and targeted with a high degree of reliability.

Because radionuclides are constantly decaying, the time spent handling the material between isolation and administration to the subject is of great importance. It would also be of considerable value if the alpha-emitting thorium nuclei could be complexed, targeted and/or administered in a form which was quick and convenient to prepare, preferably requiring few steps, short incubation periods and/or temperatures not irreversibly affecting the properties of the targeting entity.

Furthermore, processes which can be conducted in solvents that do not need removal before administration (essentially in aqueous solution) have the considerable advantage of avoiding a solvent evaporation or dialysis step.

It would also be considered of significant value if a thorium labelled drug product formulation could be developed which demonstrated significantly enhanced stability. This is critical to ensure that robust product quality standards are adhered to while at the same time enabling a logistical path to delivering patient doses. Thus formulations with minimal radiolysis over a period of 1-4 days are preferred.

Octadentate chelating agents containing hydroxypyridinone groups have previously been shown to be suitable for coordinating the alpha emitter thorium-277, for subsequent attachment to a targeting moiety (WO2011098611). Octadentate chelators were described, containing four 3,2-hydroxypyridinone groups joined by linker groups to an amine-based scaffold, having a separate reactive group used for conjugation to a targeting molecule. Preferred structures of the previous invention contained 3,2-hydroxypyridinone groups and employed the isothiocyanate moiety as the preferred coupling chemistry to the antibody component as shown in compound ALG-DD-NCS. The isothiocyanate is widely used to attach a label to proteins via amine groups. The isothiocyanate group reacts with amino terminal and primary amines in proteins and has been used for the labelling of many proteins including antibodies. Although the thiourea bond formed in these conjugates is reasonably stable, it has been reported that antibody conjugates prepared from fluorescent isothiocyanates deteriorate over time. [Banks P R, Paquette D M., Bioconjug Chem (1995) 6:447-458]. The thiourea formed by the reaction of fluorescein isothiocyanate with amines is also susceptible to conversion to a guanidine under basic conditions [Dubey I, Pratviel G, Meunier B Journal: Bioconjug Chem (1998) 9:627-632]. Due to the long decay half-life of thorium-227 (18.7 days) coupled to the long biological half-life of a monoclonal antibody it is desirable to use more stable linking moieties so as to generate conjugates which are more chemically stable both in vivo and to storage.

Work on conjugation of hydroxypyridinone ligands was published in WO2013/167754 and discloses ligands possessing a water solubilising moiety comprising a hydroxyalkyl functionality.

Due to the reactivity of the hydroxyl groups of this chelate class activation as an activated ester is not possible as multiple competing reactions ensue leading to a complex mixture of products through esterification reactions. The ligands of WO2013/167754 must therefore be coupled to the tissue-targeting protein via alternative chemistries such as the isothiocyanate giving a less stable thiourea conjugate as described above. In addition WO2013167755 and WO2013167756 discloses the hydroxyalkyl/isothiocyanate conjugates applied to CD33 and CD22 targeted antibodies respectively. Furthermore, although the introduction of the alcohol groups do contribute to water solubilization by increasing the hydrophilicity of the conjugates they are still hydrophobic enough to effect the pharmacokinetics of the labeled conjugate with significant liver uptake observed in some cases particularly in applications utilizing small molecules such as peptides.

WO2013/022797 discloses a PSMA-targeting peptide and linking structure for use with beta-emitting radionuclides. The application discloses a number of specific chelators suitable for use with the peptide, but does not suggest the use of alpha-emitters, nor the use of HOPO chelators.

WO2015/055318 discloses an alternative PSMA-targeting peptide (PSMA-617) and linking structure for use with beta-emitting radionuclides. The application discloses a number of specific chelators suitable for use with the peptide, but does not suggest the use of alpha-emitters, nor the use of HOPO chelators.

WO2016/096843 discloses 3,2-HOPO chelators radiolabeled with thorium and attached to a variety of tissue-targeted moieties. However, while such chelators are effective in the therapeutic setting, when labeled with a zirconium ion for imaging purposes, such chelators can experience π-π stacking which can lead to unwanted agglomeration.

However, the state of the art does not describe the chelators of general formula (I) of the present invention as described and defined herein.

It has now been found, and this constitutes the basis of the present invention, that the compounds of the present invention have surprising and advantageous properties.

In particular, the compounds of the present invention have surprisingly been found to be suitable for use with tissue targeting moieties, such as peptides and monoclonal antibodies and antibody fragments with reduced dimerization or agglomeration. Without being bound by one particular theory, the inventors believe the reduced dimerization or agglomeration is the result of reduction of π-π stacking.

Further, the compounds of the present invention have surprisingly been found to effectively address several biological targets associated with tumor growth.

Further, the compounds of the present invention have surprisingly been found to effectively target PSMA and may therefore be used for the treatment or prophylaxis of oncologic disorders, such as prostate cancer, for example.

### DESCRIPTION OF THE INVENTION

In accordance with a first aspect, the present invention covers compounds of general formula (I):

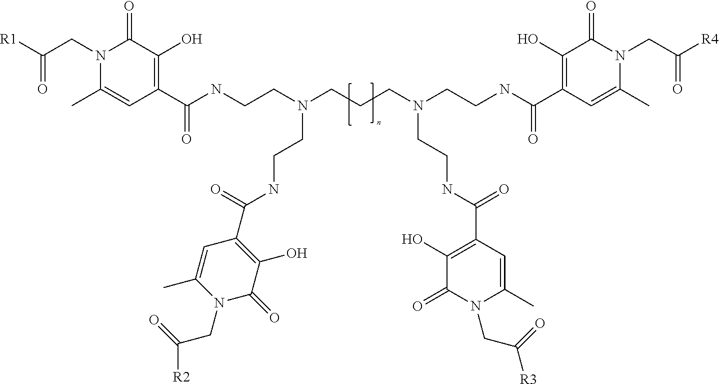

wherein:
   n is 1, 2 or 3;
   R1, R2, R3 and R4, independently represent OH or Q; and
   Q represents a tissue-targeting moeity selected from the group consisting of poly- and oligo-peptides, proteins, DNA and RNA fragments, aptamers polyclonal or monoclonal antibodies, and a mixture of proteins or fragments or constructs of protein

or a stereoisomer, a hydrate, a solvate, or a salt thereof, or a mixture of same.

In one embodiment of the first aspect of the invention, Q represents the following structure:

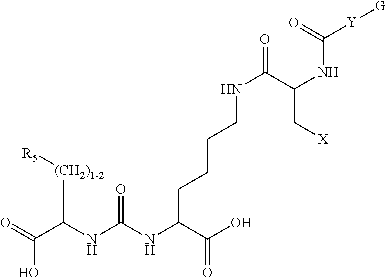

wherein X represents aryl, heteroaryl, butylurea, fluoro-substituted phenyl, butyl-substituted phenyl, quinolinyl and 2-napthyl;
wherein Y represents aryl, heteroaryl, aralkyl, hetaralkyl, C₃-C₈-cycloalkyl, or pyridine;
wherein G represents CH₂N*H or N*H with * being the attachment point to the remainder of compound (1);
and wherein R₅ represents azole, —SO₂H, —SO₃H, —SO₄H, —PO₂H, —PO₃H₂, —PO₃H, —PO₄H₂, —CO₂H, —C(O)R; wherein
R represents —H, —OH, —(C₁-C₁₀)alkyl, —O(C₁-C₁₀)alkyl, —NHR₆, or NR₆R₇;
R₆, R₇ and R₈ each independently represent H, bond, (C₁-C₁₀)alkylene, F, Cl, BR, I, C(O), C(S), —C(S)—NH-benzyl-, —C(O)—NH-benzyl-, —C(O)—(C₁-C₁₀)alkylene, —(CH₂)v-NR₈, —(CH₂)p-NH—C(O)—(CH₂)p-, —(CH₂—CH₂)t-NH—C(O)—(CH₂)p-, —(CH₂)p-COR, —(CH₂)p-C(O)NH—C[(CH₂)p-COR]₃, —C[(CH₂)p-COR]₃, or —(CH₂)p-(C₅-C₁₄)heteroaryl;
wherein v, p and t are independently 0, 1, 2, 3, 4, 5, 6, 7, 8, 9, or 10.

In another embodiment of the first aspect of the invention, Q represents a monoclonal antibody with binding affinity for targets selected from the list consisting of FAP, HER2, and PSMA.

In accordance with a second aspect, the present invention covers compounds wherein compound (1) is radiolabled with a radionuclide A selected from the group consisting of ⁴³Sc, ⁴⁴Sc, ⁴⁷Sc, ⁸⁹Zr, ⁹⁰Y, ¹¹¹In, ¹⁴⁹Tb, ¹⁵²Tb, ¹⁵⁵Tb, ¹⁶¹Tb, ¹⁶⁶Ho, ¹⁷⁷Lu, ¹⁸⁶Re, ¹⁸⁸Re, ²¹²Bi, ²¹³Bi, ²²⁵Ac, ²²⁷Th, and ²³²Th. In one embodiment, the radionuclide A is chelated according to the general structure:

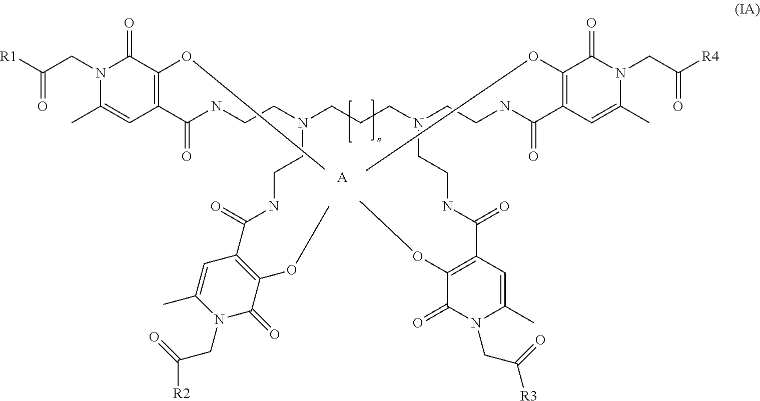

wherein:
n is 1, 2 or 3;
R1, R2, R3 and R4, independently represent OH or Q; and
Q represents a tissue-targeting moeity selected from the group consisting of poly- and oligo-peptides, proteins, DNA and RNA fragments, aptamers polyclonal or monoclonal antibodies, and a mixture of proteins or fragments or constructs of protein

or a stereoisomer, a hydrate, a solvate, or a salt thereof, or a mixture of same.

In one embodiment of the second aspect of the present invention, n Q represents the following structure:

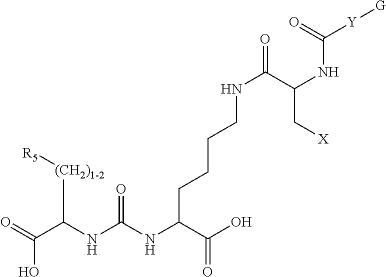

wherein X represents aryl, heteroaryl, butylurea, fluoro-substituted phenyl, butyl-substituted phenyl, quinolinyl and 2-napthyl;
wherein Y represents aryl, heteroaryl, aralkyl, hetaralkyl, C₃-C₈-cycloalkyl, or pyridine;
wherein G represents CH₂N*H or N*H with * being the attachment point to the remainder of compound (1);
and wherein R₅ represents azole, —SO₂H, —SO₃H, —SO₄H, —PO₂H, —PO₃H₂, —PO₃H, —PO₄H₂, —CO₂H, —C(O)R; wherein
R represents —H, —OH, —(C₁-C₁₀)alkyl, —O(C₁-C₁₀)alkyl, —NHR₆, or NR₆R₇;
R₆, R₇ and R₈ each independently represent H, bond, (C₁-C₁₀)alkylene, F, Cl, BR, I, C(O), C(S), —C(S)—NH-benzyl-, —C(O)—NH-benzyl-, —C(O)—(C₁-C₁₀)alkylene, —(CH₂)v-NR₈, —(CH₂)p-NH—C(O)—(CH₂)p-, —(CH₂—CH₂)t-NH—C(O)—(CH₂)p-, —(CH₂)p-COR, —(CH₂)p-C(O)NH—C[(CH₂)p-COR]₃, —C[(CH₂)p-COR]₃, or —(CH₂)p-(C₅-C₁₄)heteroaryl;
wherein v, p and t are independently 0, 1, 2, 3, 4, 5, 6, 7, 8, 9, or 10.

In accordance with an embodiment of the third aspect, the present invention covers compounds wherein compound (1) is radiolabled with a radionuclide A selected from the group consisting of of ⁴³Sc, ⁴⁴Sc, ⁴⁷Sc, ⁸⁹Zr, ⁹⁰Y, ¹¹¹In, ¹⁴⁹Tb, ¹⁵²Tb, ¹⁵⁵Tb, ¹⁶¹Tb, ¹⁶⁶Ho, ¹⁷⁷Lu, ¹⁸⁶Re, ¹⁸⁸Re, ²¹²Bi, ²¹³Bi, ²²⁵Ac, ²²⁷Th, and ²³²Th. In one embodiment, the radionuclide A is chelated according to the general structure

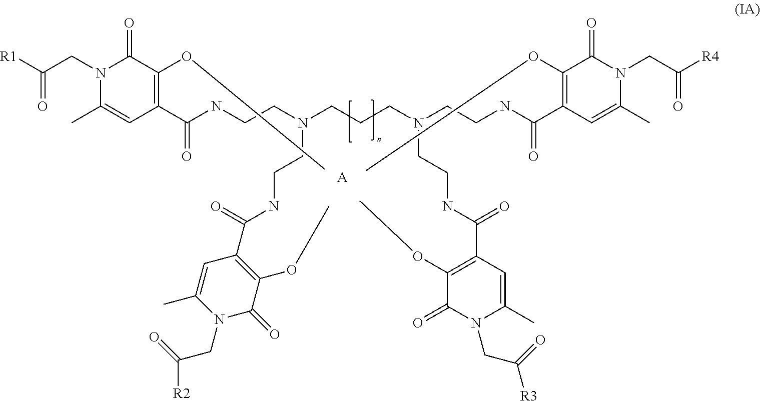

wherein Q represents a monoclonal antibody with binding affinity for targets selected from the list consisting of FAP, HER2, and PSMA.

### Definitions

The term “substituted” means that one or more hydrogen atoms on the designated atom or group are replaced with a selection from the indicated group, provided that the designated atom's normal valency under the existing circumstances is not exceeded. Combinations of substituents and/or variables are permissible.

The term “optionally substituted” means that the number of substituents can be equal to or different from zero. Unless otherwise indicated, it is possible that optionally substituted groups are substituted with as many optional substituents as can be accommodated by replacing a hydrogen atom with a non-hydrogen substituent on any available carbon or nitrogen atom.

Commonly, it is possible for the number of optional substituents, when present, to be 1, 2, 3, 4 or 5, in particular 1, 2 or 3.

As used herein, the term “one or more”, e.g. in the definition of the substituents of the compounds of general formula (I) of the present invention, means “1, 2, 3, 4 or 5, particularly 1, 2, 3 or 4, more particularly 1, 2 or 3, even more particularly 1 or 2”.

As used herein, an oxo substituent represents an oxygen atom, which is bound to a carbon atom or to a sulfur atom via a double bond.

The term “ring substituent” means a substituent attached to an aromatic or nonaromatic ring which replaces an available hydrogen atom on the ring.

The term “comprising” when used in the specification includes “consisting of”.

If within the present text any item is referred to as “as mentioned herein”, it means that it may be mentioned anywhere in the present text.

The terms as mentioned in the present text have the following meanings:

The term “halogen atom” means a fluorine, chlorine, bromine or iodine atom, particularly a fluorine, chlorine or bromine atom.

The term “C₁-C₆-alkyl” means a linear or branched, saturated, monovalent hydrocarbon group having 1, 2, 3, 4, 5 or 6 carbon atoms, e.g. a methyl, ethyl, propyl, isopropyl, butyl, sec-butyl, isobutyl, tert-butyl, pentyl, isopentyl, 2-methylbutyl, 1-methylbutyl, 1-ethylpropyl, 1,2-dimethylpropyl, neo-pentyl, 1,1-dimethylpropyl, hexyl, 1-methylpentyl, 2-methylpentyl, 3-methylpentyl, 4-methylpentyl, 1-ethylbutyl, 2-ethylbutyl, 1,1-dimethylbutyl, 2,2-dimethylbutyl, 3,3-dimethylbutyl, 2,3-dimethylbutyl, 1,2-dimethylbutyl or 1,3-dimethylbutyl group, or an isomer thereof. Particularly, said group has 1, 2, 3 or 4 carbon atoms (“C₁-C₄-alkyl”), e.g. a methyl, ethyl, propyl, isopropyl, butyl, sec-butyl isobutyl, or tert-butyl group, more particularly 1, 2 or 3 carbon atoms (“C₁-C₃-alkyl”), e.g. a methyl, ethyl, n-propyl or isopropyl group.

The term “C₁-C₈-hydroxyalkyl” means a linear or branched, saturated, monovalent hydrocarbon group in which the term “C₁-C₈-alkyl” is defined supra, and in which 1, 2 or 3 hydrogen atoms are replaced with a hydroxy group, e.g. a hydroxymethyl, 1-hydroxyethyl, 2-hydroxyethyl, 1,2-dihydroxyethyl, 3-hydroxypropyl, 2-hydroxypropyl, 1-hydroxypropyl, 1-hydroxypropan-2-yl, 2-hydroxypropan-2-yl, 2,3-dihydroxypropyl, 1,3-dihydroxypropan-2-yl, 3-hydroxy-2-methyl-propyl, 2-hydroxy-2-methyl-propyl, 1-hydroxy-2-methyl-propyl group.

The term “C₁-C₆-alkylsulfanyl” means a linear or branched, saturated, monovalent group of formula (C₁-C₆-alkyl)-S—, in which the term “C₁-C₆-alkyl” is as defined supra, e.g. a methylsulfanyl, ethylsulfanyl, propylsulfanyl, isopropylsulfanyl, butylsulfanyl, sec-butylsulfanyl, isobutylsulfanyl, tert-butylsulfanyl, pentylsulfanyl, isopentylsulfanyl, hexylsulfanyl group.

The term “C₁-C₅-haloalkyl” means a linear or branched, saturated, monovalent hydrocarbon group in which the term “C₁-C₅-alkyl” is as defined supra, and in which one or more of the hydrogen atoms are replaced, identically or differently, with a halogen atom. Particularly, said halogen atom is a fluorine atom. Said C₁-C₅-haloalkyl group is, for example, fluoromethyl, difluoromethyl, trifluoromethyl, 2-fluoroethyl, 2,2-difluoroethyl, 2,2,2-trifluoroethyl, pentafluoroethyl, 3,3,3-trifluoropropyl or 1,3-difluoropropan-2-yl.

The term “C₁-C₅-alkoxy” means a linear or branched, saturated, monovalent group of formula (C₁-C₆-alkyl)-O—, in which the term “C₁-C₆-alkyl” is as defined supra, e.g. a methoxy, ethoxy, n-propoxy, isopropoxy, n-butoxy, sec-butoxy, isobutoxy, tert-butoxy, pentyloxy, isopentyloxy or n-hexyloxy group, or an isomer thereof.

The term “C₁-C₅-haloalkoxy” means a linear or branched, saturated, monovalent C₁-C₆-alkoxy group, as defined supra, in which one or more of the hydrogen atoms is replaced, identically or differently, with a halogen atom. Particularly, said halogen atom is a fluorine atom. Said C₁-C₆-haloalkoxy group is, for example, fluoromethoxy, difluoromethoxy, trifluoromethoxy, 2,2,2-trifluoroethoxy or pentafluoroethoxy.

The term “C₂-C₆-alkenyl” means a linear or branched, monovalent hydrocarbon group, which contains one or two double bonds, and which has 2, 3, 4, 5 or 6 carbon atoms, particularly 2 or 3 carbon atoms (“C₂-C₃-alkenyl”), it being understood that in the case in which said alkenyl group contains more than one double bond, then it is possible for said double bonds to be isolated from, or conjugated with, each other. Said alkenyl group is, for example, an ethenyl (or “vinyl”), prop-2-en-1-yl (or “allyl”), prop-1-en-1-yl, but-3-enyl, but-2-enyl, but-1-enyl, pent-4-enyl, pent-3-enyl, pent-2-enyl, pent-1-enyl, hex-5-enyl, hex-4-enyl, hex-3-enyl, hex-2-enyl, hex-1-enyl, prop-1-en-2-yl (or “isopropenyl”), 2-methylprop-2-enyl, 1-methylprop-2-enyl, 2-methylprop-1-enyl, 1-methylprop-1-enyl, 3-methylbut-3-enyl, 2-methylbut-3-enyl, 1-methylbut-3-enyl, 3-methylbut-2-enyl, 2-methylbut-2-enyl, 1-methylbut-2-enyl, 3-methylbut-1-enyl, 2-methylbut-1-enyl, 1-methylbut-1-enyl, 1,1-dimethylprop-2-enyl, 1-ethylprop-1-enyl, 1-propylvinyl, 1-isopropylvinyl, 4-methylpent-4-enyl, 3-methylpent-4-enyl, 2-methylpent-4-enyl, 1-methylpent-4-enyl, 4-methylpent-3-enyl, 3-methylpent-3-enyl, 2-methylpent-3-enyl, 1-methylpent-3-enyl, 4-methylpent-2-enyl, 3-methylpent-2-enyl, 2-methylpent-2-enyl, 1-methylpent-2-enyl, 4-methylpent-1-enyl, 3-methylpent-1-enyl, 2-methylpent-1-enyl, 1-methylpent-1-enyl, 3-ethylbut-3-enyl, 2-ethylbut-3-enyl, 1-ethylbut-3-enyl, 3-ethylbut-2-enyl, 2-ethylbut-2-enyl, 1-ethylbut-2-enyl, 3-ethylbut-1-enyl, 2-ethylbut-1-enyl, 1-ethylbut-1-enyl, 2-propylprop-2-enyl, 1-propylprop-2-enyl, 2-isopropylprop-2-enyl, 1-isopropylprop-2-enyl, 2-propylprop-1-enyl, 1-propylprop-1-enyl, 2-isopropylprop-1-enyl, 1-isopropylprop-1-enyl, 3,3-dimethylprop-1-enyl, 1-(1,1-dimethylethyl)ethenyl, buta-1,3-dienyl, penta-1,4-dienyl or hexa-1,5-dienyl group. Particularly, said group is vinyl or allyl.

The term “C₂-C₆-alkynyl” means a linear or branched, monovalent hydrocarbon group which contains one triple bond, and which contains 2, 3, 4, 5 or 6 carbon atoms, particularly 2 or 3 carbon atoms (“C₂-C₃-alkynyl”). Said C₂-C₆-alkynyl group is, for example, ethynyl, prop-1-ynyl, prop-2-ynyl (or “propargyl”), but-1-ynyl, but-2-ynyl, but-3-ynyl, pent-1-ynyl, pent-2-ynyl, pent-3-ynyl, pent-4-ynyl, hex-1-ynyl, hex-2-ynyl, hex-3-ynyl, hex-4-ynyl, hex-5-ynyl, 1-methylprop-2-ynyl, 2-methylbut-3-ynyl, 1-methylbut-3-ynyl, 1-methylbut-2-ynyl, 3-methylbut-1-ynyl, 1-ethylprop-2-ynyl, 3-methylpent-4-ynyl, 2-methylpent-4-ynyl, 1-methyl-pent-4-ynyl, 2-methylpent-3-ynyl, 1-methylpent-3-ynyl, 4-methylpent-2-ynyl, 1-methyl-pent-2-ynyl, 4-methylpent-1-ynyl, 3-methylpent-1-ynyl, 2-ethylbut-3-ynyl, 1-ethylbut-3-ynyl, 1-ethylbut-2-ynyl, 1-propylprop-2-ynyl, 1-isopropylprop-2-ynyl, 2,2-dimethylbut-3-ynyl, 1,1-dimethylbut-3-ynyl, 1,1-dimethylbut-2-ynyl or 3,3-dimethylbut-1-ynyl group. Particularly, said alkynyl group is ethynyl, prop-1-ynyl or prop-2-ynyl.

The term “C₃-C₈-cycloalkyl” means a saturated, monovalent, mono- or bicyclic hydrocarbon ring which contains 3, 4, 5, 6, 7 or 8 carbon atoms (“C₃-C₈-cycloalkyl”). Said C₃-C₈-cycloalkyl group is for example, a monocyclic hydrocarbon ring, e.g. a cyclopropyl, cyclobutyl, cyclopentyl, cyclohexyl, cycloheptyl or cyclooctyl group, or a bicyclic hydrocarbon ring, e.g. a bicyclo[4.2.0]octyl or octahydropentalenyl.

The term “C₄-C₈-cycloalkenyl” means a monovalent, mono- or bicyclic hydrocarbon ring which contains 4, 5, 6, 7 or 8 carbon atoms and one double bond. Particularly, said ring contains 4, 5 or 6 carbon atoms (“C₄-C₆-cycloalkenyl”). Said C₄-C₈-cycloalkenyl group is for example, a monocyclic hydrocarbon ring, e.g. a cyclobutenyl, cyclopentenyl, cyclohexenyl, cycloheptenyl or cyclooctenyl group, or a bicyclic hydrocarbon ring, e.g. a bicyclo[2.2.1]hept-2-enyl or bicyclo[2.2.2]oct-2-enyl.

The term “C₃-C₈-cycloalkoxy” means a saturated, monovalent, mono- or bicyclic group of formula (C₃-C₈-cycloalkyl)-O—, which contains 3, 4, 5, 6, 7 or 8 carbon atoms, in which the term “C₃-C₈-cycloalkyl” is defined supra, e.g. a cyclopropyloxy, cyclobutyloxy, cyclopentyloxy, cyclohexyloxy, cycloheptyloxy or cyclooctyloxy group.

The term “spirocycloalkyl” means a saturated, monovalent bicyclic hydrocarbon group in which the two rings share one common ring carbon atom, and wherein said bicyclic hydrocarbon group contains 5, 6, 7, 8, 9, 10 or 11 carbon atoms, it being possible for said spirocycloalkyl group to be attached to the rest of the molecule via any one of the carbon atoms except the spiro carbon atom. Said spirocycloalkyl group is, for example, spiro[2.2]pentyl, spiro[2.3]hexyl, spiro[2.4]heptyl, spiro[2.5]octyl, spiro[2.6]nonyl, spiro[3.3]heptyl, spiro[3.4]octyl, spiro[3.5]nonyl, spiro[3.6]decyl, spiro[4.4]nonyl, spiro[4.5]decyl, spiro[4.6]undecyl or spiro[5.5]undecyl.

The terms “4- to 7-membered heterocycloalkyl” and “4- to 6-membered heterocycloalkyl” mean a monocyclic, saturated heterocycle with 4, 5, 6 or 7 or, respectively, 4, 5 or 6 ring atoms in total, which contains one or two identical or different ring heteroatoms from the series N, O and S, it being possible for said heterocycloalkyl group to be attached to the rest of the molecule via any one of the carbon atoms or, if present, a nitrogen atom.

Said heterocycloalkyl group, without being limited thereto, can be a 4-membered ring, such as azetidinyl, oxetanyl or thietanyl, for example; or a 5-membered ring, such as tetrahydrofuranyl, 1,3-dioxolanyl, thiolanyl, pyrrolidinyl, imidazolidinyl, pyrazolidinyl, 1,1-dioxidothiolanyl, 1,2-oxazolidinyl, 1,3-oxazolidinyl or 1,3-thiazolidinyl, for example; or a 6-membered ring, such as tetrahydropyranyl, tetrahydrothiopyranyl, piperidinyl, morpholinyl, dithianyl, thiomorpholinyl, piperazinyl, 1,3-dioxanyl, 1,4-dioxanyl or 1,2-oxazinanyl, for example, or a 7-membered ring, such as azepanyl, 1,4-diazepanyl or 1,4-oxazepanyl, for example.

Particularly, “4- to 6-membered heterocycloalkyl” means a 4- to 6-membered heterocycloalkyl as defined supra containing one ring nitrogen atom and optionally one further ring heteroatom from the series: N, O, S. More particularly, “5- or 6-membered heterocycloalkyl” means a monocyclic, saturated heterocycle with 5 or 6 ring atoms in total, containing one ring nitrogen atom and optionally one further ring heteroatom from the series: N, O.

The term “5- to 8-membered heterocycloalkenyl” means a monocyclic, unsaturated, nonaromatic heterocycle with 5, 6, 7 or 8 ring atoms in total, which contains one or two double bonds and one or two identical or different ring heteroatoms from the series: N, O, S; it being possible for said heterocycloalkenyl group to be attached to the rest of the molecule via any one of the carbon atoms or, if present, a nitrogen atom.

Said heterocycloalkenyl group is, for example, 4H-pyranyl, 2H-pyranyl, 2,5-dihydro-1H-pyrrolyl, [1,3]dioxolyl, 4H-[1,3,4]thiadiazinyl, 2,5-dihydrofuranyl, 2,3-dihydrofuranyl, 2,5-dihydrothiophenyl, 2,3-dihydrothiophenyl, 4,5-dihydrooxazolyl or 4H-[1,4]thiazinyl.

The term “heterospirocycloalkyl” means a bicyclic, saturated heterocycle with 6, 7, 8, 9, 10 or 11 ring atoms in total, in which the two rings share one common ring carbon atom, which “heterospirocycloalkyl” contains one or two identical or different ring heteroatoms from the series: N, O, S; it being possible for said heterospirocycloalkyl group to be attached to the rest of the molecule via any one of the carbon atoms, except the spiro carbon atom, or, if present, a nitrogen atom.

Said heterospirocycloalkyl group is, for example, azaspiro[2.3]hexyl, azaspiro[3.3]heptyl, oxaazaspiro[3.3]heptyl, thiaazaspiro[3.3]heptyl, oxaspiro[3.3]heptyl, oxazaspiro[5.3]nonyl, oxazaspiro[4.3]octyl, azaspiro[4,5]decyl, oxazaspiro [5.5]undecyl, diazaspiro[3.3]heptyl, thiazaspiro[3.3]heptyl, thiazaspiro[4.3]octyl, azaspiro[5.5]undecyl, or one of the further homologous scaffolds such as spiro[3.4]-, spiro[4.4]-, spiro[2.4]-, spiro[2.5]-, spiro[2.6]-, spiro[3.5]-, spiro[3.6]-, spiro[4.5]- and spiro[4.6]-.

The term “fused heterocycloalkyl” means a bicyclic, saturated heterocycle with 6, 7, 8, 9 or 10 ring atoms in total, in which the two rings share two adjacent ring atoms, which “fused heterocycloalkyl” contains one or two identical or different ring heteroatoms from the series: N, O, S; it being possible for said fused heterocycloalkyl group to be attached to the rest of the molecule via any one of the carbon atoms or, if present, a nitrogen atom.

Said fused heterocycloalkyl group is, for example, azabicyclo[3.3.0]octyl, azabicyclo[4.3.0]nonyl, diazabicyclo[4.3.0]nonyl, oxazabicyclo[4.3.0]nonyl, thiazabicyclo[4.3.0]nonyl or azabicyclo[4.4.0]decyl.

The term “bridged heterocycloalkyl” means a bicyclic, saturated heterocycle with 7, 8, 9 or 10 ring atoms in total, in which the two rings share two common ring atoms which are not adjacent, which “bridged heterocycloalkyl” contains one or two identical or different ring heteroatoms from the series: N, O, S; it being possible for said bridged heterocycloalkyl group to be attached to the rest of the molecule via any one of the carbon atoms, except the spiro carbon atom, or, if present, a nitrogen atom.

Said bridged heterocycloalkyl group is, for example, azabicyclo[2.2.1]heptyl, oxazabicyclo[2.2.1]heptyl, thiazabicyclo[2.2.1]heptyl, diazabicyclo[2.2.1]heptyl, azabicyclo-[2.2.2]octyl, diazabicyclo[2.2.2]octyl, oxazabicyclo[2.2.2]octyl, thiazabicyclo[2.2.2]octyl, azabicyclo[3.2.1]octyl, diazabicyclo[3.2.1]octyl, oxazabicyclo[3.2.1]octyl, thiazabicyclo[3.2.1]octyl, azabicyclo[3.3.1]nonyl, diazabicyclo[3.3.1]nonyl, oxazabicyclo[3.3.1]nonyl, thiazabicyclo[3.3.1]-nonyl, azabicyclo[4.2.1]nonyl, diazabicyclo[4.2.1]nonyl, oxazabicyclo[4.2.1]nonyl, thiazabicyclo[4.2.1]nonyl, azabicyclo[3.3.2]decyl, diazabicyclo[3.3.2]decyl, oxazabicyclo[3.3.2]decyl, thiazabicyclo[3.3.2]decyl or azabicyclo[4.2.2]decyl.

The term “heteroaryl” means a monovalent, monocyclic, bicyclic or tricyclic aromatic ring having 5, 6, 8, 9, 10, 11, 12, 13 or 14 ring atoms (a “5- to 14-membered heteroaryl” group), particularly 5, 6, 9 or 10 ring atoms, which contains at least one ring heteroatom and optionally one, two or three further ring heteroatoms from the series: N, O and/or S, and which is bound via a ring carbon atom or optionally via a ring nitrogen atom (if allowed by valency).

Said heteroaryl group can be a 5-membered heteroaryl group, such as, for example, thienyl, furanyl, pyrrolyl, oxazolyl, thiazolyl, imidazolyl, pyrazolyl, isoxazolyl, isothiazolyl, oxadiazolyl, triazolyl, thiadiazolyl or tetrazolyl; or a 6-membered heteroaryl group, such as, for example, pyridinyl, pyridazinyl, pyrimidinyl, pyrazinyl or triazinyl; or a tricyclic heteroaryl group, such as, for example, carbazolyl, acridinyl or phenazinyl; or a 9-membered heteroaryl group, such as, for example, benzofuranyl, benzothienyl, benzoxazolyl, benzisoxazolyl, benzimidazolyl, benzothiazolyl, benzotriazolyl, indazolyl, indolyl, isoindolyl, indolizinyl or purinyl; or a 10-membered heteroaryl group, such as, for example, quinolinyl, quinazolinyl, isoquinolinyl, cinnolinyl, phthalazinyl, quinoxalinyl or pteridinyl.

In general, and unless otherwise mentioned, the heteroaryl or heteroarylene groups include all possible isomeric forms thereof, e.g.: tautomers and positional isomers with respect to the point of linkage to the rest of the molecule. Thus, for some illustrative non-restricting examples, the term pyridinyl includes pyridin-2-yl, pyridin-3-yl and pyridin-4-yl; or the term thienyl includes thien-2-yl and thien-3-yl.

Particularly, the heteroaryl group is a quinolinyl group.

The term “azole” includes imidazoles, pyrazoles, triazoles and tetrazoles.

The term “C₁-C₆”, as used in the present text, e.g. in the context of the definition of “C₁-C₆-alkyl”, “C₁-C₆-haloalkyl”, “C₁-C₆-hydroxyalkyl”, “C₁-C₆-alkoxy” or “C₁-C₆-haloalkoxy” means an alkyl group having a finite number of carbon atoms of 1 to 6, i.e. 1, 2, 3, 4, 5 or 6 carbon atoms.

Further, as used herein, the term “C₃-C₈”, as used in the present text, e.g. in the context of the definition of “C₃-C₈-cycloalkyl”, means a cycloalkyl group having a finite number of carbon atoms of 3 to 8, i.e. 3, 4, 5, 6, 7 or 8 carbon atoms.

When a range of values is given, said range encompasses each value and sub-range within said range.

For example:

“C₁-C₆” encompasses C₁, C₂, C₃, C₄, C₅, C₆, C₁-C₆, C₁-C₆, C₁-C₄, C₁-C₃, C₁-C₂, C₂- C₆, C₂-C₅, C₂-C₄, C₂-C₃, C₃-C₆, C₃-C₅, C₃-C₄, C₄-C₆, C₄-C₅, and C₅-C₆;

“C₂-C₆” encompasses C₂, C₃, C₄, C₅, C₆, C₂-C₆, C₂-C₅, C₂-C₄, C₂-C₃, C₃-C₆, C₃-C₅, C₃-C₄, C₄-C₆, C₄-C₅, and C₅-C₆;

“C₃-C₁₀” encompasses C₃, C₄, C₅, C₆, C₇, C₈, C₉, C₁₀, C₃-C₁₀, C₃-C₉, C₃-C₈, C₃-C₇, C₃-C₆, C₃-C₅, C₃-C₄, C₄-C₁₀, C₄-C₉, C₄-C₈, C₄-C₇, C₄-C₆, C₄-C₅, C₅-C₁₀, C₅-C₉, C₅-C₈, C₅-C₇, C₅-C₆, C₆-C₁₀, C₆-C₉, C₆-C₈, C₆-C₇, C₇-C₁₀, C₇-C₉, C₇-C₈, C₈-C₁₀, C₈-C₉ and C₉-C₁₀;

“C₃-C₈” encompasses C₃, C₄, C₅, C₆, C₇, C₈, C₃-C₈, C₃-C₇, C₃-C₆, C₃-C₅, C₃-C₄, C₄- C₈, C₄-C₇, C₄-C₆, C₄-C₅, C₅-C₈, C₅-C₇, C₅-C₆, C₆-C₈, C₆-C₇ and C₇-C₈;

“C₃-C₆” encompasses C₃, C₄, C₅, C₆, C₃-C₆, C₃-C₅, C₃-C₄, C₄-C₆, C₄-C₅, and C₅-C₆;

“C₄-C₈” encompasses C₄, C₅, C₆, C₇, C₈, C₄-C₈, C₄-C₇, C₄-C₆, C₄-C₅, C₅-C₈, C₅-C₇, C₅-C₆, C₆-C₈, C₆-C₇ and C₇-C₈;

“C₄-C₇” encompasses C₄, C₅, C₆, C₇, C₄-C₇, C₄-C₆, C₄-C₅, C₅-C₇, C₅-C₆ and C₆-C₇;

“C₄-C₆” encompasses C₄, C₅, C₆, C₄-C₆, C₄-C₅ and C₅-C₆;

“C₅-C₁₀” encompasses C₅, C₆, C₇, C₈, C₉, C₁₀, C₅-C₁₀, C₅-C₉, C₅-C₈, C₅-C₇, C₅-C₆, C₆- C₁₀, C₆-C₉, C₆-C₈, C₆-C₇, C₇-C₁₀, C₇-C₉, C₇-C₈, C₈-C₁₀, C₈-C₉ and C₉-C₁₀;

“C₆-C₁₀” encompasses C₆, C₇, C₈, C₉, C₁₀, C₆-C₁₀, C₆-C₉, C₆-C₈, C₆-C₇, C₇-C₁₀, C₇-C₉, C₇-C₈, C₈-C₁₀, C₈-C₉ and C₉-C₁₀.

As used herein, the term “leaving group” means an atom or a group of atoms that is displaced in a chemical reaction as stable species taking with it the bonding electrons. In particular, such a leaving group is selected from the group comprising: halide, in particular fluoride, chloride, bromide or iodide, (methylsulfonyl)oxy, [(trifluoromethyl)sulfonyl]oxy, [(nonafluorobutyl)-sulfonyl]oxy, (phenylsulfonyl)oxy, [(4-methylphenyl)sulfonyl]oxy, [(4-bromophenyl)sulfonyl]oxy, [(4-nitrophenyl)sulfonyl]oxy, [(2-nitrophenyl)sulfonyl]oxy, [(4-isopropylphenyl)sulfonyl]oxy, [(2,4,6-triisopropylphenyl)sulfonyl]oxy, [(2,4,6-trimethylphenyl)sulfonyl]oxy, [(4-tert-butyl-phenyl)sulfonyl]oxy and [(4-methoxyphenyl)sulfonyl]oxy.

The schemes and procedures described below illustrate synthetic routes to the compounds of formula (I) of the invention and are not intended to be limiting. It is obvious to the person skilled in the art that the order of transformations as exemplified in the Schemes can be modified in various ways. The order of transformations exemplified in the Schemes is therefore not intended to be limiting. In addition, interconversion of any of the substituents can be achieved before and/or after the exemplified transformations. These modifications can be such as the introduction of protecting groups, cleavage of protecting groups, reduction or oxidation of functional groups, halogenation, metallation, substitution or other reactions known to the person skilled in the art. These transformations include those which introduce a functionality which allows for further interconversion of substituents. Appropriate protecting groups and their introduction and cleavage are well-known to the person skilled in the art (see for example T. W. Greene and P. G. M. Wuts in Protective Groups in Organic Synthesis, 3rd edition, Wiley 1999). Specific examples are described in the subsequent paragraphs.

It is possible for the compounds of general formula (I) to exist as isotopic variants. The invention therefore includes one or more isotopic variant(s) of the compounds of general formula (I), particularly deuterium-containing compounds of general formula (I).

The term “Isotopic variant” of a compound or a reagent is defined as a compound exhibiting an unnatural proportion of one or more of the isotopes that constitute such a compound.

The term “Isotopic variant of the compound of general formula (I)” is defined as a compound of general formula (I) exhibiting an unnatural proportion of one or more of the isotopes that constitute such a compound.

The expression “unnatural proportion” means a proportion of such isotope which is higher than its natural abundance. The natural abundances of isotopes to be applied in this context are described in “Isotopic Compositions of the Elements 1997”, Pure Appl. Chem., 70(1), 217-235, 1998.

Examples of such isotopes include stable and radioactive isotopes of hydrogen, carbon, nitrogen, oxygen, phosphorus, sulfur, fluorine, chlorine, bromine and iodine, such as ²H (deuterium), ³H (tritium), ¹¹C, ¹³C, ¹⁴C, ¹⁵N, ¹⁷O, ¹⁸O, ³²P, ³³P, ³³S, ³⁴S, ³⁵S, ³⁶S, ¹⁸F, ³⁶Cl, ⁸²Br, ¹²³I, ¹²⁴, ¹²⁵I, ¹²⁹I and ¹³¹I, respectively.

With respect to the treatment and/or prophylaxis of the disorders specified herein the isotopic variant(s) of the compounds of general formula (I) preferably contain deuterium (“deuterium-containing compounds of general formula (I)”). Isotopic variants of the compounds of general formula (I) in which one or more radioactive isotopes, such as ³H or ¹⁴C, are incorporated are useful e.g. in drug and/or substrate tissue distribution studies. These isotopes are particularly preferred for the ease of their incorporation and detectability. Positron emitting isotopes such as ¹⁸F or ¹¹C may be incorporated into a compound of general formula (I). These isotopic variants of the compounds of general formula (I) are useful for in vivo imaging applications. Deuterium-containing and ¹³C-containing compounds of general formula (I) can be used in mass spectrometry analyses (H. J. Leis et al., Curr. Org. Chem., 1998, 2, 131) in the context of preclinical or clinical studies.

Isotopic variants of the compounds of general formula (I) can generally be prepared by methods known to a person skilled in the art, such as those described in the schemes and/or examples herein, by substituting a reagent for an isotopic variant of said reagent, preferably for a deuterium-containing reagent. Depending on the desired sites of deuteration, in some cases deuterium from D₂O can be incorporated either directly into the compounds or into reagents that are useful for synthesizing such compounds (Esaki et al., Tetrahedron, 2006, 62, 10954; Esaki et al., Chem. Eur. J., 2007, 13, 4052). Deuterium gas is also a useful reagent for incorporating deuterium into molecules. Catalytic deuteration of olefinic bonds (H. J. Leis et al., Curr. Org. Chem., 1998, 2, 131; J. R. Morandi et al., J. Org. Chem., 1969, 34 (6), 1889) and acetylenic bonds (N. H. Khan, J. Am. Chem. Soc., 1952, 74 (12), 3018; S. Chandrasekhar et al., Tetrahedron Letters, 2011, 52, 3865) is a rapid route for incorporation of deuterium. Metal catalysts (i.e. Pd, Pt, and Rh) in the presence of deuterium gas can be used to directly exchange deuterium for hydrogen in functional groups containing hydrocarbons (J. G. Atkinson et al., U.S. Pat. No. 3,966,781). A variety of deuterated reagents and synthetic building blocks are commercially available from companies such as for example C/D/N Isotopes, Quebec, Canada; Cambridge Isotope Laboratories Inc., Andover, Mass., USA; and CombiPhos Catalysts, Inc., Princeton, N.J., USA. Further information on the state of the art with respect to deuterium-hydrogen exchange is given for example in Hanzlik et al., J. Org. Chem. 55, 3992-3997, 1990; R. P. Hanzlik et al., Biochem. Biophys. Res. Commun. 160, 844, 1989; P. J. Reider et al., J. Org. Chem. 52, 3326-3334, 1987; M. Jarman et al., Carcinogenesis 16(4), 683-688, 1995; J. Atzrodt et al., Angew. Chem., Int. Ed. 2007, 46, 7744; K. Matoishi et al., Chem. Commun. 2000, 1519-1520; K. Kassahun et al., WO2012/112363.

The term “deuterium-containing compound of general formula (I)” is defined as a compound of general formula (I), in which one or more hydrogen atom(s) is/are replaced by one or more deuterium atom(s) and in which the abundance of deuterium at each deuterated position of the compound of general formula (I) is higher than the natural abundance of deuterium, which is about 0.015%. Particularly, in a deuterium-containing compound of general formula (I) the abundance of deuterium at each deuterated position of the compound of general formula (I) is higher than 10%, 20%, 30%, 40%, 50%, 60%, 70% or 80%, preferably higher than 90%, 95%, 96% or 97%, even more preferably higher than 98% or 99% at said position(s). It is understood that the abundance of deuterium at each deuterated position is independent of the abundance of deuterium at other deuterated position(s).

The selective incorporation of one or more deuterium atom(s) into a compound of general formula (I) may alter the physicochemical properties (such as for example acidity [C. L. Perrin, et al., J. Am. Chem. Soc., 2007, 129, 4490; A. Streitwieser et al., J. Am. Chem. Soc., 1963, 85, 2759;], basicity [C. L. Perrin et al., J. Am. Chem. Soc., 2005, 127, 9641; C. L. Perrin, et al., J. Am. Chem. Soc., 2003, 125, 15008; C. L. Perrin in Advances in Physical Organic Chemistry, 44, 144], lipophilicity [B. Testa et al., Int. J. Pharm., 1984, 19(3), 271]) and/or the metabolic profile of the molecule and may result in changes in the ratio of parent compound to metabolites or in the amounts of metabolites formed. Such changes may result in certain therapeutic advantages and hence may be preferred in some circumstances. Reduced rates of metabolism and metabolic switching, where the ratio of metabolites is changed, have been reported (A. E. Mutlib et al., Toxicol. Appl. Pharmacol., 2000, 169, 102; D. J. Kushner et al., Can. J. Physiol. Pharmacol., 1999, 77, 79). These changes in the exposure to parent drug and metabolites can have important consequences with respect to the pharmacodynamics, tolerability and efficacy of a deuterium-containing compound of general formula (I). In some cases deuterium substitution reduces or eliminates the formation of an undesired or toxic metabolite and enhances the formation of a desired metabolite (e.g. Nevirapine: A. M. Sharma et al., Chem. Res. Toxicol., 2013, 26, 410; Efavirenz: A. E. Mutlib et al., Toxicol. Appl. Pharmacol., 2000, 169, 102). In other cases the major effect of deuteration is to reduce the rate of systemic clearance.

As a result, the biological half-life of the compound is increased. The potential clinical benefits would include the ability to maintain similar systemic exposure with decreased peak levels and increased trough levels. This could result in lower side effects and enhanced efficacy, depending on the particular compound's pharmacokinetic/pharmacodynamic relationship. ML-337 (C. J. Wenthur et al., J. Med. Chem., 2013, 56, 5208) and Odanacatib (K. Kassahun et al., WO2012/112363) are examples for this deuterium effect. Still other cases have been reported in which reduced rates of metabolism result in an increase in exposure of the drug without changing the rate of systemic clearance (e.g. Rofecoxib: F. Schneider et al., Arzneim. Forsch./Drug. Res., 2006, 56, 295; Telaprevir: F. Maltais et al., J. Med. Chem., 2009, 52, 7993). Deuterated drugs showing this effect may have reduced dosing requirements (e.g. lower number of doses or lower dosage to achieve the desired effect) and/or may produce lower metabolite loads.

A compound of general formula (I) may have multiple potential sites of attack for metabolism. To optimize the above-described effects on physicochemical properties and metabolic profile, deuterium-containing compounds of general formula (I) having a certain pattern of one or more deuterium-hydrogen exchange(s) can be selected. Particularly, the deuterium atom(s) of deuterium-containing compound(s) of general formula (I) is/are attached to a carbon atom and/or is/are located at those positions of the compound of general formula (I), which are sites of attack for metabolizing enzymes such as e.g. cytochrome P₄₅₀.

Where the plural form of the word compounds, salts, polymorphs, hydrates, solvates and the like, is used herein, this is taken to mean also a single compound, salt, polymorph, isomer, hydrate, solvate or the like.

By “stable compound” or “stable structure” is meant a compound that is sufficiently robust to survive isolation to a useful degree of purity from a reaction mixture, and formulation into an efficacious therapeutic agent.

The compounds of the present invention optionally contain one or more asymmetric centres, depending upon the location and nature of the various substituents desired. It is possible that one or more asymmetric carbon atoms are present in the (R) or (S) configuration, which can result in racemic mixtures in the case of a single asymmetric centre, and in diastereomeric mixtures in the case of multiple asymmetric centres. In certain instances, it is possible that asymmetry also be present due to restricted rotation about a given bond, for example, the central bond adjoining two substituted aromatic rings of the specified compounds.

Preferred compounds are those which produce the more desirable biological activity. Separated, pure or partially purified isomers and stereoisomers or racemic or diastereomeric mixtures of the compounds of the present invention are also included within the scope of the present invention. The purification and the separation of such materials can be accomplished by standard techniques known in the art.

The optical isomers can be obtained by resolution of the racemic mixtures according to conventional processes, for example, by the formation of diastereoisomeric salts using an optically active acid or base or formation of covalent diastereomers. Examples of appropriate acids are tartaric, diacetyltartaric, ditoluoyltartaric and camphorsulfonic acid. Mixtures of diastereoisomers can be separated into their individual diastereomers on the basis of their physical and/or chemical differences by methods known in the art, for example, by chromatography or fractional crystallisation. The optically active bases or acids are then liberated from the separated diastereomeric salts. A different process for separation of optical isomers involves the use of chiral chromatography (e.g., HPLC columns using a chiral phase), with or without conventional derivatisation, optimally chosen to maximise the separation of the enantiomers. Suitable HPLC columns using a chiral phase are commercially available, such as those manufactured by Daicel, e.g., Chiracel OD and Chiracel OJ, for example, among many others, which are all routinely selectable. Enzymatic separations, with or without derivatisation, are also useful. The optically active compounds of the present invention can likewise be obtained by chiral syntheses utilizing optically active starting materials.

In order to distinguish different types of isomers from each other reference is made to IUPAC Rules Section E (Pure Appl Chem 45, 11-30, 1976).

The present invention includes all possible stereoisomers of the compounds of the present invention as single stereoisomers, or as any mixture of said stereoisomers, e.g. (R)- or (S)-isomers, in any ratio. Isolation of a single stereoisomer, e.g. a single enantiomer or a single diastereomer, of a compound of the present invention is achieved by any suitable state of the art method, such as chromatography, especially chiral chromatography, for example.

Further, it is possible for the compounds of the present invention to exist as tautomers. For example, any compound of the present invention which contains an imidazopyridine moiety as a heteroaryl group for example can exist as a 1H tautomer, or a 3H tautomer, or even a mixture in any amount of the two tautomers, namely:

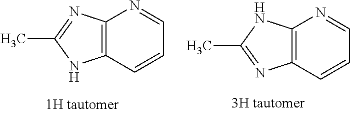

The present invention includes all possible tautomers of the compounds of the present invention as single tautomers, or as any mixture of said tautomers, in any ratio.

Further, the compounds of the present invention can exist as N-oxides, which are defined in that at least one nitrogen of the compounds of the present invention is oxidised. The present invention includes all such possible N-oxides.

The present invention also covers useful forms of the compounds of the present invention, such as metabolites, hydrates, solvates, prodrugs, salts, in particular pharmaceutically acceptable salts, and/or co-precipitates.

The compounds of the present invention can exist as a hydrate, or as a solvate, wherein the compounds of the present invention contain polar solvents, in particular water, methanol or ethanol for example, as structural element of the crystal lattice of the compounds. It is possible for the amount of polar solvents, in particular water, to exist in a stoichiometric or non-stoichiometric ratio. In the case of stoichiometric solvates, e.g. a hydrate, hemi-, (semi-), mono-, sesqui-, di-, tri-, tetra-, penta- etc. solvates or hydrates, respectively, are possible. The present invention includes all such hydrates or solvates.

Further, it is possible for the compounds of the present invention to exist in free form, e.g. as a free base, or as a free acid, or as a zwitterion, or to exist in the form of a salt. Said salt may be any salt, either an organic or inorganic addition salt, particularly any pharmaceutically acceptable organic or inorganic addition salt, which is customarily used in pharmacy, or which is used, for example, for isolating or purifying the compounds of the present invention.

The term “pharmaceutically acceptable salt” refers to an inorganic or organic acid addition salt of a compound of the present invention. For example, see S. M. Berge, et al. “Pharmaceutical Salts,” J. Pharm. Sci. 1977, 66, 1-19.

A suitable pharmaceutically acceptable salt of the compounds of the present invention may be, for example, an acid-addition salt of a compound of the present invention bearing a nitrogen atom, in a chain or in a ring, for example, which is sufficiently basic, such as an acid-addition salt with an inorganic acid, or “mineral acid”, such as hydrochloric, hydrobromic, hydroiodic, sulfuric, sulfamic, bisulfuric, phosphoric, or nitric acid, for example, or with an organic acid, such as formic, acetic, acetoacetic, pyruvic, trifluoroacetic, propionic, butyric, hexanoic, heptanoic, undecanoic, lauric, benzoic, salicylic, 2-(4-hydroxybenzoyl)-benzoic, camphoric, cinnamic, cyclopentanepropionic, digluconic, 3-hydroxy-2-naphthoic, nicotinic, pamoic, pectinic, 3-phenylpropionic, pivalic, 2-hydroxyethanesulfonic, itaconic, trifluoromethanesulfonic, dodecylsulfuric, ethanesulfonic, benzenesulfonic, para-toluenesulfonic, methanesulfonic, 2-naphthalenesulfonic, naphthalinedisulfonic, camphorsulfonic acid, citric, tartaric, stearic, lactic, oxalic, malonic, succinic, malic, adipic, alginic, maleic, fumaric, D-gluconic, mandelic, ascorbic, glucoheptanoic, glycerophosphoric, aspartic, sulfosalicylic, or thiocyanic acid, for example.

Further, another suitably pharmaceutically acceptable salt of a compound of the present invention which is sufficiently acidic, is an alkali metal salt, for example a sodium or potassium salt, an alkaline earth metal salt, for example a calcium, magnesium or strontium salt, or an aluminium or a zinc salt, or an ammonium salt derived from ammonia or from an organic primary, secondary or tertiary amine having 1 to 20 carbon atoms, such as ethylamine, diethylamine, triethylamine, ethyldiisopropylamine, monoethanolamine, diethanolamine, triethanolamine, dicyclohexylamine, dimethylaminoethanol, diethylaminoethanol, tris(hydroxymethyl)aminomethane, procaine, dibenzylamine, N-methylmorpholine, arginine, lysine, 1,2-ethylenediamine, N-methylpiperidine, N-methyl-glucamine, N,N-dimethyl-glucamine, N-ethyl-glucamine, 1,6-hexanediamine, glucosamine, sarcosine, serinol, 2-amino-1,3-propanediol, 3-amino-1,2-propanediol, 4-amino-1,2,3-butanetriol, or a salt with a quaternary ammonium ion having 1 to 20 carbon atoms, such as tetramethylammonium, tetraethylammonium, tetra(n-propyl)ammonium, tetra(n-butyl)ammonium, N-benzyl-N,N,N-trimethylammonium, choline or benzalkonium.

Those skilled in the art will further recognise that it is possible for acid addition salts of the claimed compounds to be prepared by reaction of the compounds with the appropriate inorganic or organic acid via any of a number of known methods. Alternatively, alkali and alkaline earth metal salts of acidic compounds of the present invention are prepared by reacting the compounds of the present invention with the appropriate base via a variety of known methods.

The present invention includes all possible salts of the compounds of the present invention as single salts, or as any mixture of said salts, in any ratio.

In the present text, in particular in the Experimental Section, for the synthesis of intermediates and of examples of the present invention, when a compound is mentioned as a salt form with the corresponding base or acid, the exact stoichiometric composition of said salt form, as obtained by the respective preparation and/or purification process, is, in most cases, unknown.

Unless specified otherwise, suffixes to chemical names or structural formulae relating to salts, such as “hydrochloride”, “trifluoroacetate”, “sodium salt”, or “x HCl”, “x CF₃OOOH”, “x Na*”, for example, mean a salt form, the stoichiometry of which salt form not being specified.

This applies analogously to cases in which synthesis intermediates or example compounds or salts thereof have been obtained, by the preparation and/or purification processes described, as solvates, such as hydrates, with (if defined) unknown stoichiometric composition.

Furthermore, the present invention includes all possible crystalline forms, or polymorphs, of the compounds of the present invention, either as single polymorph, or as a mixture of more than one polymorph, in any ratio.

Moreover, the present invention also includes prodrugs of the compounds according to the invention. The term “prodrugs” here designates compounds which themselves can be biologically active or inactive, but are converted (for example metabolically or hydrolytically) into compounds according to the invention during their residence time in the body.

Suitable targeting moieties include poly- and oligo-peptides, proteins, DNA and RNA fragments, aptamers etc, preferably a protein, e.g. avidin, streptavidin, a polyclonal or monoclonal antibody (including IgG and IgM type antibodies), or a mixture of proteins or fragments or constructs of protein. Antibodies, antibody constructs, fragments of antibodies (e.g. Fab fragments or any fragment comprising at least one antigen binding region(s)), constructs of fragments (e.g. single chain antibodies) or a mixture thereof are particularly preferred. Suitable fragments particularly include Fab, F(ab′)₂, Fab′ and/or scFv. Antibody constructs may be of any antibody or fragment indicated herein.

In accordance with an embodiment of the first aspect, the present invention covers compounds of general formula (I), supra, in wherein Q represents the following structure:

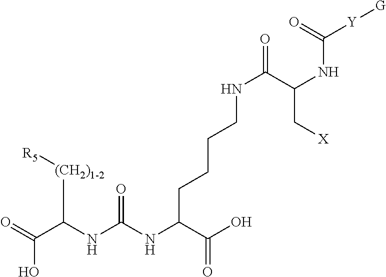

wherein X represents aryl, heteroaryl, butylurea, fluoro-substituted phenyl, butyl-substituted phenyl, quinolinyl and 2-napthyl;
wherein Y represents aryl, heteroaryl, aralkyl, hetaralkyl, C₃-C₈-cycloalkyl, or pyridine; wherein G represents CH₂N*H or N*H with * being the attachment point to the remainder of compound (I);
and wherein R₅ represents tetrazole, —SO₂H, —SO₃H, —SO₄H, —PO₂H, —PO₃H₂, —PO₃H, —PO₄H₂, —CO₂H, —C(O)R; wherein
R represents —H, —OH, —(C₁-C₁₀)alkyl, —O(C₁-C₁₀)alkyl, —NHR₆, or NR₆R₇;
R₆, R₇ and R₈ each independently represent H, bond, (C₁-C₁₀)alkylene, F, Cl, BR, I, C(O), C(S), —C(S)—NH-benzyl-, —C(O)—NH-benzyl-, —C(O)—(C₁-C₁₀)alkylene, —(CH₂)v-NR₈, —(CH₂)p-NH—C(O)—(CH₂)p-, —(CH₂—CH₂)t-NH—C(O)—(CH₂)p-, —(CH₂)p-COR, —(CH₂)p-C(O)NH—C[(CH₂)p-COR]₃, —C[(CH₂)p-COR]₃, or —(CH₂)p-(C₅-C₁₄)heteroaryl;
wherein v, p and t are independently 0, 1, 2, 3, 4, 5, 6, 7, 8, 9, or 10.

or a stereoisomer, a hydrate, a solvate, or a salt thereof, or a mixture of same.

In accordance with an alternate embodiment of the second aspect, the present invention covers compounds of general formula (IA), supra, in which:

wherein compound (1) is radiolabled with a radionuclide A selected from the group consisting of ⁴³Sc, ⁴⁴Sc, ⁴⁷Sc, ⁸⁹Zr, ⁹⁰Y, ¹¹¹In, ¹⁴⁹Tb, ¹⁵²Tb, ¹⁵⁵Tb, ¹⁶¹Tb, ¹⁶⁶Ho, ¹⁷⁷Lu, ¹⁸⁶Re, ¹⁸⁸Re, ²¹²Bi, ²¹³Bi, ²²⁵Ac, ²²⁷Th, and ²³²Th:

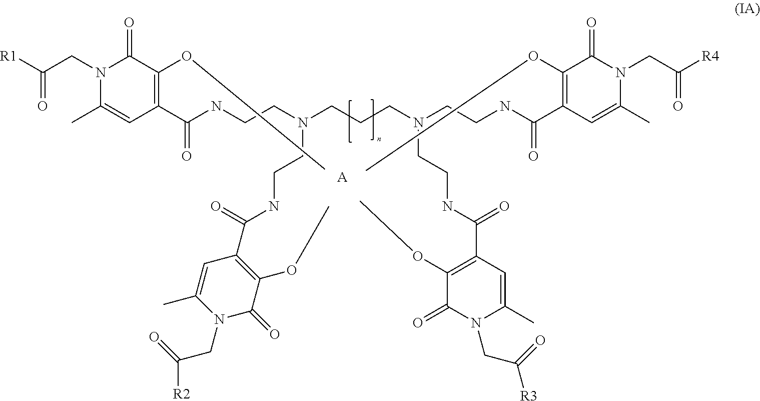

wherein Q represents the following structure:

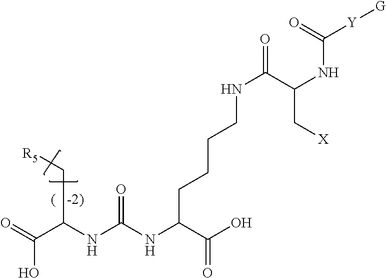

wherein X represents aryl, heteroaryl, butylurea, fluoro-substituted phenyl, butyl-substituted phenyl, quinoline and 2-napthyl;
wherein Y represents aryl, heteroaryl, aralkyl, hetaralkyl, C₃-C₈-cycloalkyl, or pyridine; wherein G represents CH₂N*H or N*H with * being the attachment point to the remainder of compound (1);
and wherein R5 represents R₅ represents —SO₂H, —SO₃H, —SO₄H, —PO₂H, —PO₃H₂, —PO₃H, —PO₄H₂, —CO₂H, —C(O)R; wherein
R represents —H, —OH, —(C₁-C₁₀)alkyl, —O(C₁-C₁₀)alkyl, —NHR₆, or NR₆R₇;
R₆, R₇ and R₈ each independently represent H, bond, (C₁-C₁₀)alkylene, F, Cl, BR, I, C(O), C(S), —C(S)—NH-benzyl-, —C(O)—NH-benzyl-, —C(O)—(C₁-C₁₀)alkylene, —(CH₂)v-NR₈, —(CH₂)p-NH—C(O)—(CH₂)p-, —(CH₂—CH₂)t-NH—C(O)—(CH₂)p-, —(CH₂)p-COR, —(CH₂)p-C(O)NH—C[(CH₂)p-COR]₃, —C[(CH₂)p-COR]₃, or —(CH₂)p-(C₅-C₁₄)heteroaryl;
wherein v, p and t are independently 0, 1, 2, 3, 4, 5, 6, 7, 8, 9, or 10;

or a stereoisomer, a hydrate, a solvate, or a salt thereof, or a mixture of same.

In accordance with an alternate embodiment of the second aspect, the present invention covers compounds of general formula (I), supra, in which:
n is 1;
two of R1, R2, R3 and R4 represent OH and two of R1, R2, R3 and R4 represent Q; or a stereoisomer, a hydrate, a solvate, or a salt thereof, or a mixture of same. It should be noted in this embodiment, both constitutional isomers (1,1 and 1,2) can both be present.

In accordance with an alternate embodiment of the second aspect, the present invention covers compounds of general formula (I), supra, in which:
n is 1;
three of R1, R2, R3 and R4 represent OH and one of R1, R2, R3 and R4 represents Q, or a stereoisomer, a hydrate, a solvate, or a salt thereof, or a mixture of same.

In accordance with an alternate embodiment of the second aspect, the present invention covers compounds of general formula (I), supra, in which:
n is 1;
one of R1, R2, R3 and R4 represents OH and three of R1, R2, R3 and R4 represent Q,

or a stereoisomer, a hydrate, a solvate, or a salt thereof, or a mixture of same.

In accordance with an alternate embodiment of the second aspect, the present invention covers compounds of general formula (I), supra, in which:
n is 1;
all of R1, R2, R3 and R4 represent Q,

or a stereoisomer, a hydrate, a solvate, or a salt thereof, or a mixture of same

In accordance with a further embodiment of the second aspect, the present invention covers compounds of general formula (I), supra, in which:

X represents 2-quinolinyl, and Y represents pyridine, and stereoisomers, tautomers, N-oxides, hydrates, solvates, and salts thereof, and mixtures of same.

In a particular further embodiment of the first aspect, the present invention covers combinations of two or more of the above mentioned embodiments under the heading “further embodiments of the second aspect of the present invention”.

The present invention covers any sub-combination within any embodiment or aspect of the present invention of compounds of general formula (I), supra.

In accordance with an embodiment of the third aspect, the present invention covers compounds of general formula (IA) wherein compound (I) is radiolabled with a radionuclide A selected from the group consisting of ⁴³Sc, ⁴⁴Sc, ⁴⁷Sc, ⁸⁹Zr, ⁹⁰Y, ¹¹¹In, ¹⁴⁹Tb, ¹⁵²Tb, ¹⁵⁵Tb, ¹⁶¹Tb, ¹⁶⁶Ho, ¹⁷⁷Lu, ¹⁸⁶Re, ¹⁸⁸Re, ²¹²Bi, ²¹³Bi, ²²⁵Ac, ²²⁷Th, and ²³²Th

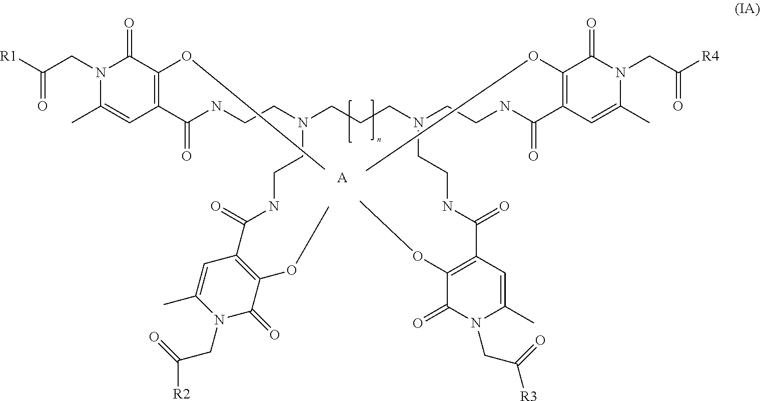

wherein Q represents a monoclonal antibody with binding affinity for targets selected from the list consisting of FAP, HER2, and PSMA.

In accordance with an alternate embodiment of the third aspect, the present invention covers compounds of general formula (I), supra, in which:
n is 1;
two of R1, R2, R3 and R4 represent OH and two of R1, R2, R3 and R4 represent Q; or a stereoisomer, a hydrate, a solvate, or a salt thereof, or a mixture of same. It should be noted in this embodiment, both constitutional isomers (1,1 and 1,2) can both be present.

In accordance with an alternate embodiment of the third aspect, the present invention covers compounds of general formula (I), supra, in which:
n is 1;
three of R1, R2, R3 and R4 represent OH and one of R1, R2, R3 and R4 represents Q, or a stereoisomer, a hydrate, a solvate, or a salt thereof, or a mixture of same.

In accordance with an alternate embodiment of the third aspect, the present invention covers compounds of general formula (I), supra, in which:
n is 1;
one of R1, R2, R3 and R4 represents OH and three of R1, R2, R3 and R4 represent Q,

or a stereoisomer, a hydrate, a solvate, or a salt thereof, or a mixture of same.

In accordance with an alternate embodiment of the third aspect, the present invention covers compounds of general formula (I), supra, in which:
n is 1;
all of R1, R2, R3 and R4 represent Q,

or a stereoisomer, a hydrate, a solvate, or a salt thereof, or a mixture of same

The present invention covers any sub-combination within any embodiment or aspect of the present invention of intermediate compounds of general formula (IA).

The present invention covers the compounds of general formula (I) which are disclosed in the Example Section of this text, infra.

The compounds of general formula (I) of the present invention can be converted to any salt, preferably pharmaceutically acceptable salts, as described herein, by any method which is known to the person skilled in the art. Similarly, any salt of a compound of general formula (I) of the present invention can be converted into the free compound, by any method which is known to the person skilled in the art.

Compounds of general formula (I) of the present invention demonstrate a valuable pharmacological spectrum of action and pharmacokinetic profile, both of which could not have been predicted. Compounds of the present invention have surprisingly been found to effectively target tumors and it is possible therefore that said compounds be used for the treatment or prophylaxis of diseases, preferably soft tissue disorders in humans and animals.

Compounds of the present invention can be utilized to inhibit, block, reduce, decrease, etc., cell proliferation and/or cell division, and/or produce apoptosis. This method comprises administering to a mammal in need thereof, including a human, an amount of a compound of general formula (I) of the present invention, or a pharmaceutically acceptable salt, isomer, polymorph, metabolite, hydrate, solvate or ester thereof, which is effective to treat the disorder.

Hyperproliferative disorders include, but are not limited to, for example: psoriasis, keloids, and other hyperplasias affecting the skin, benign prostate hyperplasia (BPH), solid tumours, such as cancers of the breast, respiratory tract, brain, reproductive organs, digestive tract, urinary tract, eye, liver, skin, head and neck, thyroid, parathyroid and their distant metastases. Those disorders also include lymphomas, sarcomas, and leukaemias.

Examples of breast cancers include, but are not limited to, invasive ductal carcinoma, invasive lobular carcinoma, ductal carcinoma in situ, and lobular carcinoma in situ.

Examples of cancers of the respiratory tract include, but are not limited to, small-cell and non-small-cell lung carcinoma, as well as bronchial adenoma and pleuropulmonary blastoma.

Examples of brain cancers include, but are not limited to, brain stem and hypothalmic glioma, cerebellar and cerebral astrocytoma, medulloblastoma, ependymoma, as well as neuroectodermal and pineal tumour.

Tumours of the male reproductive organs include, but are not limited to, prostate and testicular cancer.

Tumours of the female reproductive organs include, but are not limited to, endometrial, cervical, ovarian, vaginal, and vulvar cancer, as well as sarcoma of the uterus.

Tumours of the digestive tract include, but are not limited to, anal, colon, colorectal, oesophageal, gallbladder, gastric, pancreatic, rectal, small-intestine, and salivary gland cancers.

Tumours of the urinary tract include, but are not limited to, bladder, penile, kidney, renal pelvis, ureter, urethral and human papillary renal cancers.

Eye cancers include, but are not limited to, intraocular melanoma and retinoblastoma.

Examples of liver cancers include, but are not limited to, hepatocellular carcinoma (liver cell carcinomas with or without fibrolamellar variant), cholangiocarcinoma (intrahepatic bile duct carcinoma), and mixed hepatocellular cholangiocarcinoma.

Skin cancers include, but are not limited to, squamous cell carcinoma, Kaposi's sarcoma, malignant melanoma, Merkel cell skin cancer, and non-melanoma skin cancer.

Head-and-neck cancers include, but are not limited to, laryngeal, hypopharyngeal, nasopharyngeal, oropharyngeal cancer, lip and oral cavity cancer and squamous cell.

Lymphomas include, but are not limited to, AIDS-related lymphoma, non-Hodgkin's lymphoma, cutaneous T-cell lymphoma, Burkitt lymphoma, Hodgkin's disease, and lymphoma of the central nervous system.

Sarcomas include, but are not limited to, sarcoma of the soft tissue, osteosarcoma, malignant fibrous histiocytoma, lymphosarcoma, and rhabdomyosarcoma.

Leukemias include, but are not limited to, acute myeloid leukemia, acute lymphoblastic leukemia, chronic lymphocytic leukemia, chronic myelogenous leukemia, and hairy cell leukemia.

The present invention also provides methods of treating angiogenic disorders including diseases associated with excessive and/or abnormal angiogenesis.

Inappropriate and ectopic expression of angiogenesis can be deleterious to an organism. A number of pathological conditions are associated with the growth of extraneous blood vessels.

These include, for example, diabetic retinopathy, ischemic retinal-vein occlusion, and retinopathy of prematurity [Aiello et al., New Engl. J. Med., 1994, 331, 1480; Peer et al., Lab. Invest., 1995, 72, 638], age-related macular degeneration (AMD) [Lopez et al., Invest. Opththalmol. Vis. Sci., 1996, 37, 855], neovascular glaucoma, psoriasis, retrolental fibroplasias, angiofibroma, inflammation, rheumatoid arthritis (RA), restenosis, in-stent restenosis, vascular graft restenosis, etc. In addition, the increased blood supply associated with cancerous and neoplastic tissue, encourages growth, leading to rapid tumour enlargement and metastasis. Moreover, the growth of new blood and lymph vessels in a tumour provides an escape route for renegade cells, encouraging metastasis and the consequence spread of the cancer. Thus, compounds of general formula (I) of the present invention can be utilized to treat and/or prevent any of the aforementioned angiogenesis disorders, for example by inhibiting and/or reducing blood vessel formation; by inhibiting, blocking, reducing, decreasing, etc. endothelial cell proliferation, or other types involved in angiogenesis, as well as causing cell death or apoptosis of such cell types.

These disorders have been well characterized in humans, but also exist with a similar etiology in other mammals, and can be treated by administering pharmaceutical compositions of the present invention.

The term “treating” or “treatment” as stated throughout this document is used conventionally, for example the management or care of a subject for the purpose of combating, alleviating, reducing, relieving, improving the condition of a disease or disorder, such as a carcinoma.

The compounds of the present invention can be used in particular in therapy and prevention, i.e. prophylaxis, of tumour growth and metastases, especially in solid tumours of all indications and stages with or without pre-treatment of the tumour growth.

Generally, the use of chemotherapeutic agents and/or anti-cancer agents in combination with a compound or pharmaceutical composition of the present invention will serve to:
1. yield better efficacy in reducing the growth of a tumour or even eliminate the tumour as compared to administration of either agent alone,
2. provide for the administration of lesser amounts of the administered chemotherapeutic agents,
3. provide for a chemotherapeutic treatment that is well tolerated in the patient with fewer deleterious pharmacological complications than observed with single agent chemotherapies and certain other combined therapies,
4. provide for treating a broader spectrum of different cancer types in mammals, especially humans,
5. provide for a higher response rate among treated patients,
6. provide for a longer survival time among treated patients compared to standard chemotherapy treatments,
7. provide a longer time for tumour progression, and/or
8. yield efficacy and tolerability results at least as good as those of the agents used alone, compared to known instances where other cancer agent combinations produce antagonistic effects.

In addition, the compounds of general formula (I) of the present invention can also be used in combination with radiotherapy and/or surgical intervention.

In a further embodiment of the present invention, the compounds of general formula (I) of the present invention may be used to sensitize a cell to radiation, i.e. treatment of a cell with a compound of the present invention prior to radiation treatment of the cell renders the cell more susceptible to DNA damage and cell death than the cell would be in the absence of any treatment with a compound of the present invention. In one aspect, the cell is treated with at least one compound of general formula (I) of the present invention.

Thus, the present invention also provides a method of killing a cell, wherein a cell is administered one or more compounds of the present invention in combination with conventional radiation therapy.

The present invention also provides a method of rendering a cell more susceptible to cell death, wherein the cell is treated with one or more compounds of general formula (I) of the present invention prior to the treatment of the cell to cause or induce cell death. In one aspect, after the cell is treated with one or more compounds of general formula (I) of the present invention, the cell is treated with at least one compound, or at least one method, or a combination thereof, in order to cause DNA damage for the purpose of inhibiting the function of the normal cell or killing the cell.

In other embodiments of the present invention, a cell is killed by treating the cell with at least one DNA damaging agent, i.e. after treating a cell with one or more compounds of general formula (I) of the present invention to sensitize the cell to cell death, the cell is treated with at least one DNA damaging agent to kill the cell. DNA damaging agents useful in the present invention include, but are not limited to, chemotherapeutic agents (e.g. cis platin), ionizing radiation (X-rays, ultraviolet radiation), carcinogenic agents, and mutagenic agents.

In other embodiments, a cell is killed by treating the cell with at least one method to cause or induce DNA damage. Such methods include, but are not limited to, activation of a cell signalling pathway that results in DNA damage when the pathway is activated, inhibiting of a cell signalling pathway that results in DNA damage when the pathway is inhibited, and inducing a biochemical change in a cell, wherein the change results in DNA damage. By way of a non-limiting example, a DNA repair pathway in a cell can be inhibited, thereby preventing the repair of DNA damage and resulting in an abnormal accumulation of DNA damage in a cell.

In one aspect of the invention, a compound of general formula (I) of the present invention is administered to a cell prior to the radiation or other induction of DNA damage in the cell. In another aspect of the invention, a compound of general formula (I) of the present invention is administered to a cell concomitantly with the radiation or other induction of DNA damage in the cell. In yet another aspect of the invention, a compound of general formula (I) of the present invention is administered to a cell immediately after radiation or other induction of DNA damage in the cell has begun.

In another aspect, the cell is in vitro. In another embodiment, the cell is in vivo.

In accordance with a further aspect, the present invention covers compounds of general formula (I), as described supra, or stereoisomers, tautomers, N-oxides, hydrates, solvates, and salts thereof, particularly pharmaceutically acceptable salts thereof, or mixtures of same, for use in the diagnosis, treatment, or prophylaxis of diseases, in particular soft tissue disorders.

In accordance with a further aspect, the present invention covers the use of compounds of general formula (I), as described supra, or stereoisomers, tautomers, N-oxides, hydrates, solvates, and salts thereof, particularly pharmaceutically acceptable salts thereof, or mixtures of same, for the diagnosis, treatment, or prophylaxis of diseases, in particular soft tissue disorders, particularly prostate cancer.

In accordance with a further aspect, the present invention covers the use of a compound of formula (I), described supra, or a stereoisomer, a tautomer, an N-oxide, a hydrate, a solvate, or a salt thereof, particularly a pharmaceutically acceptable salt thereof, or a mixture of same, for the diagnosis, prophylaxis, or treatment of diseases, in particular soft tissue disorders, particularly prostate cancer.

In accordance with a further aspect, the present invention covers the use of compounds of general formula (I), as described supra, or stereoisomers, tautomers, N-oxides, hydrates, solvates, and salts thereof, particularly pharmaceutically acceptable salts thereof, or mixtures of same, in a method of diagnosis, treatment or prophylaxis of diseases, in particular soft tissue disorders, particularly prostate cancer.

In accordance with a further aspect, the present invention covers use of a compound of general formula (I), as described supra, or stereoisomers, tautomers, N-oxides, hydrates, solvates, and salts thereof, particularly pharmaceutically acceptable salts thereof, or mixtures of same, for the preparation of a pharmaceutical composition, preferably a medicament, for the diagnosis, prophylaxis, or treatment of diseases, in particular soft tissue disorders, particularly prostate cancer.

In accordance with a further aspect, the present invention covers a method of diagnosis, treatment, or prophylaxis of diseases, in particular soft tissue disorders, particularly prostate cancer, using an effective amount of a compound of general formula (I), as described supra, or stereoisomers, tautomers, N-oxides, hydrates, solvates, and salts thereof, particularly pharmaceutically acceptable salts thereof, or mixtures of same.

In a preferred embodiment, compounds of general formula (I) can be radiolabled with an appropriate radionuclide and used for the imaging of an internal organ of a mammal according to conventional methods.

In accordance with a further aspect, the present invention covers pharmaceutical compositions, in particular a medicament, comprising a compound of general formula (I), as described supra, or a stereoisomer, a tautomer, an N-oxide, a hydrate, a solvate, a salt thereof, particularly a pharmaceutically acceptable salt, or a mixture of same, and one or more excipients), in particular one or more pharmaceutically acceptable excipient(s). Conventional procedures for preparing such pharmaceutical compositions in appropriate dosage forms can be utilized.

The present invention furthermore covers pharmaceutical compositions, in particular medicaments, which comprise at least one compound according to the invention, conventionally together with one or more pharmaceutically suitable excipients, and to their use for the above mentioned purposes.

It is possible for the compounds according to the invention to have systemic and/or local activity. For this purpose, they can be administered in a suitable manner, such as, for example, via the oral, parenteral, pulmonary, nasal, sublingual, lingual, buccal, rectal, vaginal, dermal, transdermal, conjunctival, otic route or as an implant or stent.

For these administration routes, it is possible for the compounds according to the invention to be administered in suitable administration forms.

For oral administration, it is possible to formulate the compounds according to the invention to dosage forms known in the art that deliver the compounds of the invention rapidly and/or in a modified manner, such as, for example, tablets (uncoated or coated tablets, for example with enteric or controlled release coatings that dissolve with a delay or are insoluble), orally-disintegrating tablets, films/wafers, films/lyophylisates, capsules (for example hard or soft gelatine capsules), sugar-coated tablets, granules, pellets, powders, emulsions, suspensions, aerosols or solutions. It is possible to incorporate the compounds according to the invention in crystalline and/or amorphised and/or dissolved form into said dosage forms.

Parenteral administration can be effected with avoidance of an absorption step (for example intravenous, intraarterial, intracardial, intraspinal or intralumbal) or with inclusion of absorption (for example intramuscular, subcutaneous, intracutaneous, percutaneous or intraperitoneal). Administration forms which are suitable for parenteral administration are, inter alia, preparations for injection and infusion in the form of solutions, suspensions, emulsions, lyophylisates or sterile powders.

Examples which are suitable for other administration routes are pharmaceutical forms for inhalation [inter alia powder inhalers, nebulizers], nasal drops, nasal solutions, nasal sprays; tablets/films/wafers/capsules for lingual, sublingual or buccal administration; suppositories; eye drops, eye ointments, eye baths, ocular inserts, ear drops, ear sprays, ear powders, ear-rinses, ear tampons; vaginal capsules, aqueous suspensions (lotions, mixturae agitandae), lipophilic suspensions, emulsions, ointments, creams, transdermal therapeutic systems (such as, for example, patches), milk, pastes, foams, dusting powders, implants or stents.

The compounds according to the invention can be incorporated into the stated administration forms. This can be effected in a manner known per se by mixing with pharmaceutically suitable excipients. Pharmaceutically suitable excipients include, inter alia,
fillers and carriers (for example cellulose, microcrystalline cellulose (such as, for example, Avicel®), lactose, mannitol, starch, calcium phosphate (such as, for example, Di-Cafos®)),
ointment bases (for example petroleum jelly, paraffins, triglycerides, waxes, wool wax, wool wax alcohols, lanolin, hydrophilic ointment, polyethylene glycols),
bases for suppositories (for example polyethylene glycols, cacao butter, hard fat),
solvents (for example water, ethanol, isopropanol, glycerol, propylene glycol, medium chain-length triglycerides fatty oils, liquid polyethylene glycols, paraffins),
surfactants, emulsifiers, dispersants or wetters (for example sodium dodecyl sulfate), lecithin, phospholipids, fatty alcohols (such as, for example, Lanette®), sorbitan fatty acid esters (such as, for example, Span®), polyoxyethylene sorbitan fatty acid esters (such as, for example, Tween®), polyoxyethylene fatty acid glycerides (such as, for example, Cremophor®), polyoxyethylene fatty acid esters, polyoxyethylene fatty alcohol ethers, glycerol fatty acid esters, poloxamers (such as, for example, Pluronic®),
buffers, acids and bases (for example phosphates, carbonates, citric acid, acetic acid, hydrochloric acid, sodium hydroxide solution, ammonium carbonate, trometamol, triethanolamine),
isotonicity agents (for example glucose, sodium chloride),
adsorbents (for example highly-disperse silicas),
viscosity-increasing agents, gel formers, thickeners and/or binders (for example polyvinylpyrrolidone, methylcellulose, hydroxypropylmethylcellulose, hydroxypropylcellulose, carboxymethylcellulose-sodium, starch, carbomers, polyacrylic acids (such as, for example, Carbopol®); alginates, gelatine),
disintegrants (for example modified starch, carboxymethylcellulose-sodium, sodium starch glycolate (such as, for example, Explotab®), cross-linked polyvinylpyrrolidone, croscarmellose-sodium (such as, for example, AcDiSol®)),
flow regulators, lubricants, glidants and mould release agents (for example magnesium stearate, stearic acid, talc, highly-disperse silicas (such as, for example, Aerosil®)),
coating materials (for example sugar, shellac) and film formers for films or diffusion membranes which dissolve rapidly or in a modified manner (for example polyvinylpyrrolidones (such as, for example, Kollidon®), polyvinyl alcohol, hydroxypropylmethylcellulose, hydroxypropylcellulose, ethylcellulose, hydroxypropylmethylcellulose phthalate, cellulose acetate, cellulose acetate phthalate, polyacrylates, polymethacrylates such as, for example, Eudragit®)),
capsule materials (for example gelatine, hydroxypropylmethylcellulose),
synthetic polymers (for example polylactides, polyglycolides, polyacrylates, polymethacrylates (such as, for example, Eudragit®), polyvinylpyrrolidones (such as, for example, Kollidon®), polyvinyl alcohols, polyvinyl acetates, polyethylene oxides, polyethylene glycols and their copolymers and blockcopolymers),
plasticizers (for example polyethylene glycols, propylene glycol, glycerol, triacetine, triacetyl citrate, dibutyl phthalate),
penetration enhancers,
stabilisers (for example antioxidants such as, for example, ascorbic acid, ascorbyl palmitate, sodium ascorbate, butylhydroxyanisole, butylhydroxytoluene, propyl gallate),
preservatives (for example parabens, sorbic acid, thiomersal, benzalkonium chloride, chlorhexidine acetate, sodium benzoate),
colourants (for example inorganic pigments such as, for example, iron oxides, titanium dioxide),
flavourings, sweeteners, flavour- and/or odour-masking agents.

The present invention furthermore relates to a pharmaceutical composition which comprise at least one compound according to the invention, conventionally together with one or more pharmaceutically suitable excipient(s), and to their use according to the present invention.

In accordance with another aspect, the present invention covers pharmaceutical combinations, in particular medicaments, comprising at least one compound of general formula (I) of the present invention and at least one or more further active ingredients, in particular for the diagnosis, treatment, and/or prophylaxis of prostate cancer.

Particularly, the present invention covers a pharmaceutical combination, which comprises:
one or more first active ingredients, in particular compounds of general formula (I) as defined supra, and
one or more further active ingredients, in particular prostate cancer The term “combination” in the present invention is used as known to persons skilled in the art, it being possible for said combination to be a fixed combination, a non-fixed combination or a kit-of-parts.

A “fixed combination” in the present invention is used as known to persons skilled in the art and is defined as a combination wherein, for example, a first active ingredient, such as one or more compounds of general formula (I) of the present invention, and a further active ingredient are present together in one unit dosage or in one single entity. One example of a “fixed combination” is a pharmaceutical composition wherein a first active ingredient and a further active ingredient are present in admixture for simultaneous administration, such as in a formulation. Another example of a “fixed combination” is a pharmaceutical combination wherein a first active ingredient and a further active ingredient are present in one unit without being in admixture.

A non-fixed combination or “kit-of-parts” in the present invention is used as known to persons skilled in the art and is defined as a combination wherein a first active ingredient and a further active ingredient are present in more than one unit. One example of a non-fixed combination or kit-of-parts is a combination wherein the first active ingredient and the further active ingredient are present separately. It is possible for the components of the non-fixed combination or kit-of-parts to be administered separately, sequentially, simultaneously, concurrently or chronologically staggered.

The compounds of the present invention can be administered as the sole pharmaceutical agent or in combination with one or more other pharmaceutically active ingredients where the combination causes no unacceptable adverse effects. The present invention also covers such pharmaceutical combinations. For example, the compounds of the present invention can be combined with known anti-cancer agents.

Examples of anti-cancer agents include:

131I-chTNT, abarelix, abemaciclib, abiraterone, acalabrutinib, aclarubicin, adalimumab, ado-trastuzumab emtansine, afatinib, aflibercept, aldesleukin, alectinib, alemtuzumab, alendronic acid, alitretinoin, altretamine, amifostine, aminoglutethimide, hexyl aminolevulinate, amrubicin, amsacrine, anastrozole, ancestim, anethole dithiolethione, anetumab ravtansine, angiotensin II, antithrombin III, apalutamide, aprepitant, arcitumomab, arglabin, arsenic trioxide, asparaginase, atezolizumab, avelumab, axicabtagene ciloleucel, axitinib, azacitidine, basiliximab, belotecan, bendamustine, besilesomab, belinostat, bevacizumab, bexarotene, bicalutamide, bisantrene, bleomycin, blinatumomab, bortezomib, bosutinib, buserelin, brentuximab vedotin, brigatinib, busulfan, cabazitaxel, cabozantinib, calcitonine, calcium folinate, calcium levofolinate, capecitabine, capromab, carbamazepine carboplatin, carboquone, carfilzomib, carmofur, carmustine, catumaxomab, celecoxib, celmoleukin, ceritinib, cetuximab, chlorambucil, chlormadinone, chlormethine, cidofovir, cinacalcet, cisplatin, cladribine, clodronic acid, clofarabine, cobimetinib, copanlisib, crisantaspase, crizotinib, cyclophosphamide, cyproterone, cytarabine, dacarbazine, dactinomycin, daratumumab, darbepoetin alfa, dabrafenib, dasatinib, daunorubicin, decitabine, degarelix, denileukin diftitox, denosumab, depreotide, deslorelin, dianhydrogalactitol, dexrazoxane, dibrospidium chloride, dianhydrogalactitol, diclofenac, dinutuximab, docetaxel, dolasetron, doxifluridine, doxorubicin, doxorubicin+estrone, dronabinol, durvalumab, eculizumab, edrecolomab, elliptinium acetate, elotuzumab, eltrombopag, enasidenib, endostatin, enocitabine, enzalutamide, epirubicin, epitiostanol, epoetin alfa, epoetin beta, epoetin zeta, eptaplatin, eribulin, erlotinib, esomeprazole, estradiol, estramustine, ethinylestradiol, etoposide, everolimus, exemestane, fadrozole, fentanyl, filgrastim, fluoxymesterone, floxuridine, fludarabine, fluorouracil, flutamide, folinic acid, formestane, fosaprepitant, fotemustine, fulvestrant, gadobutrol, gadoteridol, gadoteric acid meglumine, gadoversetamide, gadoxetic acid, gallium nitrate, ganirelix, gefitinib, gemcitabine, gemtuzumab, Glucarpidase, glutoxim, GM-CSF, goserelin, granisetron, granulocyte colony stimulating factor, histamine dihydrochloride, histrelin, hydroxycarbamide, 1-125 seeds, lansoprazole, ibandronic acid, ibritumomab tiuxetan, ibrutinib, idarubicin, ifosfamide, imatinib, imiquimod, improsulfan, indisetron, incadronic acid, ingenol mebutate, inotuzumab ozogamicin, interferon alfa, interferon beta, interferon gamma, iobitridol, iobenguane (123I), iomeprol, ipilimumab, irinotecan, Itraconazole, ixabepilone, ixazomib, lanreotide, lansoprazole, lapatinib, lasocholine, lenalidomide, lenvatinib, lenograstim, lentinan, letrozole, leuprorelin, levamisole, levonorgestrel, levothyroxine sodium, lisuride, lobaplatin, lomustine, lonidamine, lutetium Lu 177 dotatate, masoprocol, medroxyprogesterone, megestrol, melarsoprol, melphalan, mepitiostane, mercaptopurine, mesna, methadone, methotrexate, methoxsalen, methylaminolevulinate, methylprednisolone, methyltestosterone, metirosine, midostaurin, mifamurtide, miltefosine, miriplatin, mitobronitol, mitoguazone, mitolactol, mitomycin, mitotane, mitoxantrone, mogamulizumab, molgramostim, mopidamol, morphine hydrochloride, morphine sulfate, mvasi, nabilone, nabiximols, nafarelin, naloxone+pentazocine, naltrexone, nartograstim, necitumumab, nedaplatin, nelarabine, neratinib, neridronic acid, netupitant/palonosetron, nivolumab, pentetreotide, nilotinib, nilutamide, nimorazole, nimotuzumab, nimustine, nintedanib, niraparib, nitracrine, nivolumab, obinutuzumab, octreotide, ofatumumab, olaparib, olaratumab, omacetaxine mepesuccinate, omeprazole, ondansetron, oprelvekin, orgotein, orilotimod, osimertinib, oxaliplatin, oxycodone, oxymetholone, ozogamicine, p53 gene therapy, paclitaxel, palbociclib, palifermin, palladium-103 seed, palonosetron, pamidronic acid, panitumumab, panobinostat, pantoprazole, pazopanib, pegaspargase, PEG-epoetin beta (methoxy PEG-epoetin beta), pembrolizumab, pegfilgrastim, peginterferon alfa-2b, pembrolizumab, pemetrexed, pentazocine, pentostatin, peplomycin, Perflubutane, perfosfamide, Pertuzumab, picibanil, pilocarpine, pirarubicin, pixantrone, plerixafor, plicamycin, poliglusam, polyestradiol phosphate, polyvinylpyrrolidone+sodium hyaluronate, polysaccharide-K, pomalidomide, ponatinib, porfimer sodium, pralatrexate, prednimustine, prednisone, procarbazine, procodazole, propranolol, quinagolide, rabeprazole, racotumomab, radium-223 chloride, radotinib, raloxifene, raltitrexed, ramosetron, ramucirumab, ranimustine, rasburicase, razoxane, refametinib, regorafenib, ribociclib, risedronic acid, rhenium-186 etidronate, rituximab, rolapitant, romidepsin, romiplostim, romurtide, rucaparib, samarium (153Sm) lexidronam, sargramostim, sarilumab, satumomab, secretin, siltuximab, sipuleucel-T, sizofiran, sobuzoxane, sodium glycididazole, sonidegib, sorafenib, stanozolol, streptozocin, sunitinib, talaporfin, talimogene laherparepvec, tamibarotene, tamoxifen, tapentadol, tasonermin, teceleukin, technetium (99mTc) nofetumomab merpentan, 99mTc-HYNIC-[Tyr3]-octreotide, tegafur, tegafur+gimeracil+oteracil, temoporfin, temozolomide, temsirolimus, teniposide, testosterone, tetrofosmin, thalidomide, thiotepa, thymalfasin, thyrotropin alfa, tioguanine, tisagenlecleucel, tocilizumab, topotecan, toremifene, tositumomab, trabectedin, trametinib, tramadol, trastuzumab, trastuzumab emtansine, treosulfan, tretinoin, trifluridine+tipiracil, trilostane, triptorelin, trametinib, trofosfamide, thrombopoietin, tryptophan, ubenimex, valatinib, valrubicin, vandetanib, vapreotide, vemurafenib, vinblastine, vincristine, vindesine, vinflunine, vinorelbine, vismodegib, vorinostat, vorozole, yttrium-90 glass microspheres, zinostatin, zinostatin stimalamer, zoledronic acid, zorubicin.

Based upon standard laboratory techniques known to evaluate compounds useful for the treatment of soft tissue disease, by standard toxicity tests and by standard pharmacological assays for the determination of treatment of the conditions identified above in mammals, and by comparison of these results with the results of known active ingredients or medicaments that are used to treat these conditions, the effective dosage of the compounds of the present invention can readily be determined for treatment of each desired indication. The amount of the active ingredient to be administered in the treatment of one of these conditions can vary widely according to such considerations as the particular compound and dosage unit employed, the mode of administration, the period of treatment, the age and sex of the patient treated, and the nature and extent of the condition treated.

In all aspects of the present invention, the tissue-targeting radiopharmaceutical preferably comprises Th-227. The radiopharmaceutical is preferably administered at a dosage level of thorium-227 dosage of 500 kBq/kg to 2 MBq/kg bodyweight, preferably 1.5 MBq/kg. Correspondingly, a single dosage until may comprise around any of these ranges multiplied by a suitable bodyweight, such as 30 to 150 Kg, preferably 40 to 100 Kg Of course the specific initial and continuing dosage regimen for each patient will vary according to the nature and severity of the condition as determined by the attending diagnostician, the activity of the specific compound employed, the age and general condition of the patient, time of administration, route of administration, rate of excretion of the drug, drug combinations, and the like. The desired mode of treatment and number of doses of a compound of the present invention or a pharmaceutically acceptable salt or ester or composition thereof can be ascertained by those skilled in the art using conventional treatment tests.

General Procedures

The compounds according to the invention can be prepared according to the following schemes 1 through 15.

The schemes and procedures described below illustrate synthetic routes to the compounds of formula (I) of the invention and are not intended to be limiting. It is obvious to the person skilled in the art that the order of transformations as exemplified in the Schemes can be modified in various ways. The order of transformations exemplified in the Schemes is therefore not intended to be limiting. In addition, interconversion of any of the substituents, R1, R2, R3, R4, R5, R6, R7, R8, R9, R10, R11, R12, R13, V, W, Y, Z and Het can be achieved before and/or after the exemplified transformations. These modifications can be such as the introduction of protecting groups, cleavage of protecting groups, reduction or oxidation of functional groups, halogenation, metallation, substitution or other reactions known to the person skilled in the art. These transformations include those which introduce a functionality which allows for further interconversion of substituents. Appropriate protecting groups and their introduction and cleavage are well-known to the person skilled in the art (see for example T. W. Greene and P. G. M. Wuts in Protective Groups in Organic Synthesis, 3rd edition, Wiley 1999). Specific examples are described in the subsequent paragraphs.

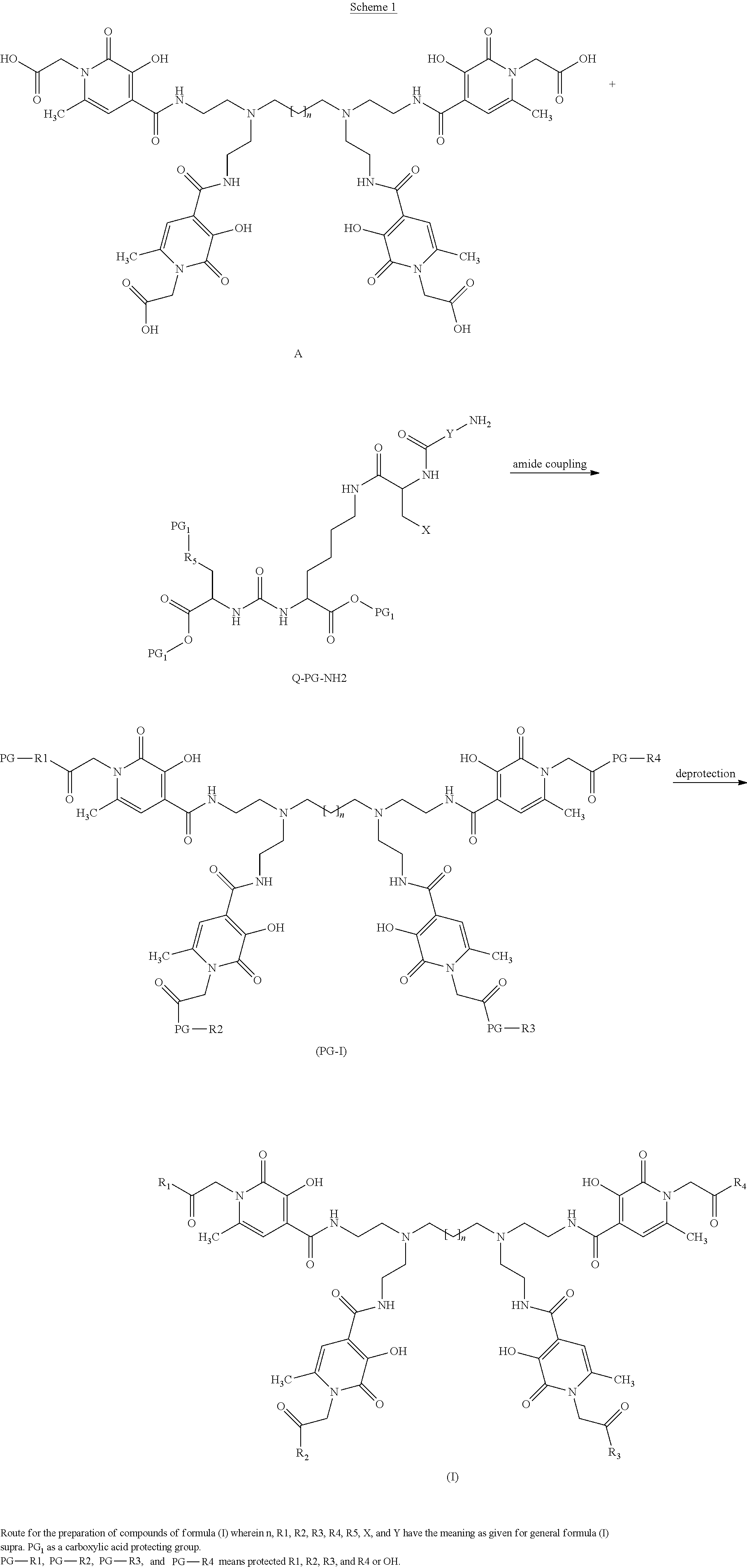

HOPO chelator A is reacted with a suitably protected amine Q-PG-NH2 under amide coupling conditions known to those skilled in the art to give amide PG-I. Possible reaction conditions include but are not limited to amide coupling reagents like HATU (1-[Bis(dimethylamino)methylene]-1H-1,2,3-triazolo[4,5-b]pyridinium 3-oxide hexafluorophosphate) or PyAOP ((3-hydroxy-3H-1,2,3-triazolo[4,5-b]pyridinato-O)tri-1-pyrrolidinyl-phosphorus hexafluorophosphate). In this reaction in theory 0-4 amidation reactions with Q-PG-NH2 can occur at HOPO chelator A yielding different multimers: monomer for one reaction, dimer for two reactions, trimer for three reactions, tetramer for four reactions at the HOPO chelator A.

In the next step the protect compound PG-I is deprotected by conditions known to those skilled in the art to give compound I. For example the removal of Boc-amine protecting groups or tert-butyl esters can be achieved by TFA (trifluoroacetic acid) or hydrochloric acid.

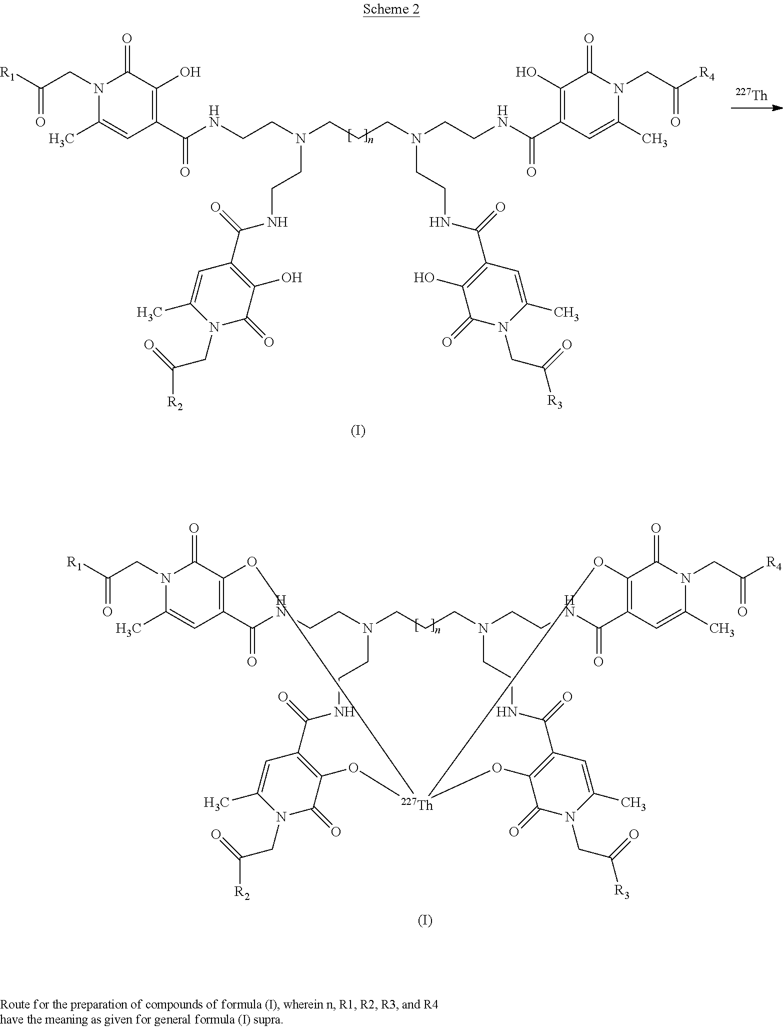

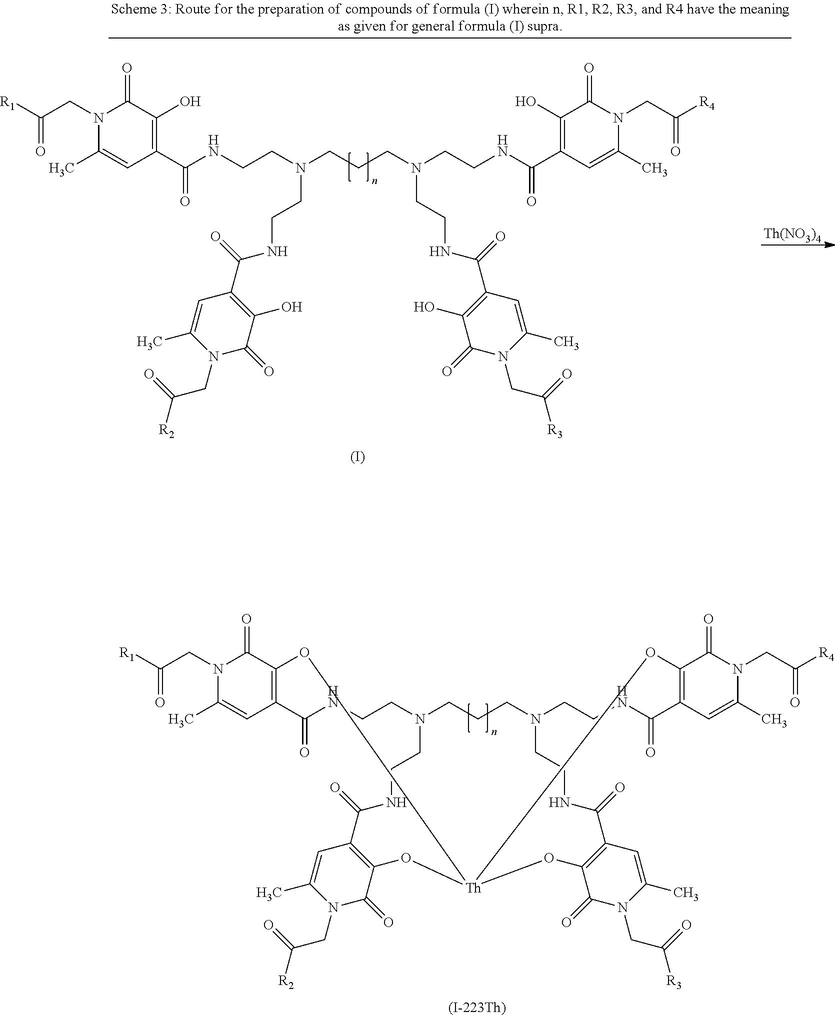

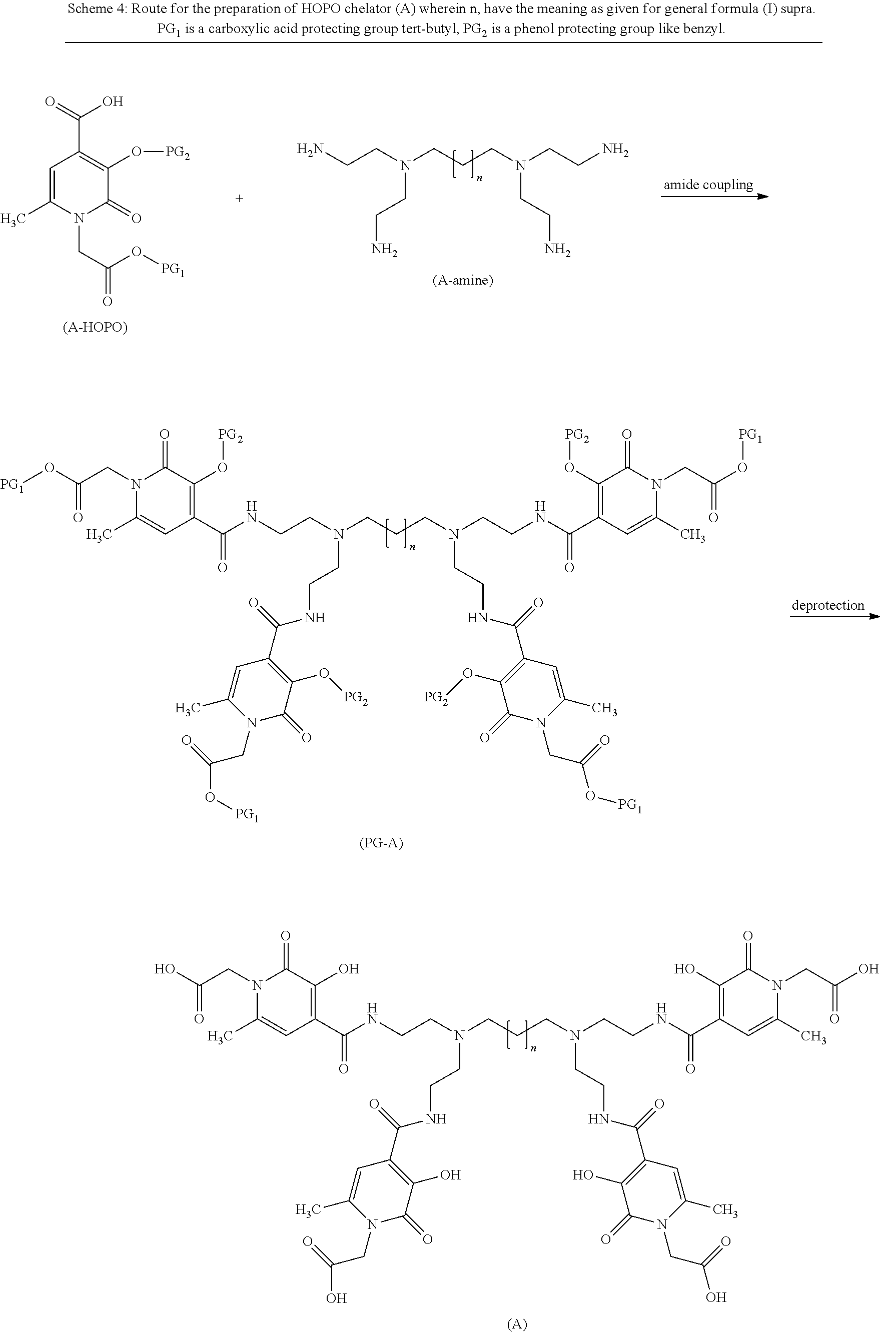

Suitably protected hydroxypyridone A-HOPO is coupled to tetraamine A-amine under amide coupling conditions known to those skilled in the art. Possible reaction conditions include but are not limited to amide coupling reagents like HATU (1-[Bis(dimethylamino)methylene]-1H-1,2,3-triazolo[4,5-b]pyridinium 3-oxide hexafluorophosphate). In the following step the protecting groups are removed by conditions known to those skilled in the art for the respective protecting groups. Possible reaction conditions include but are not limited to cleavage by hydrochloric acid, hydrobromic acid, hydrogen bromide in acetic acid or trifluoroacetic acid.

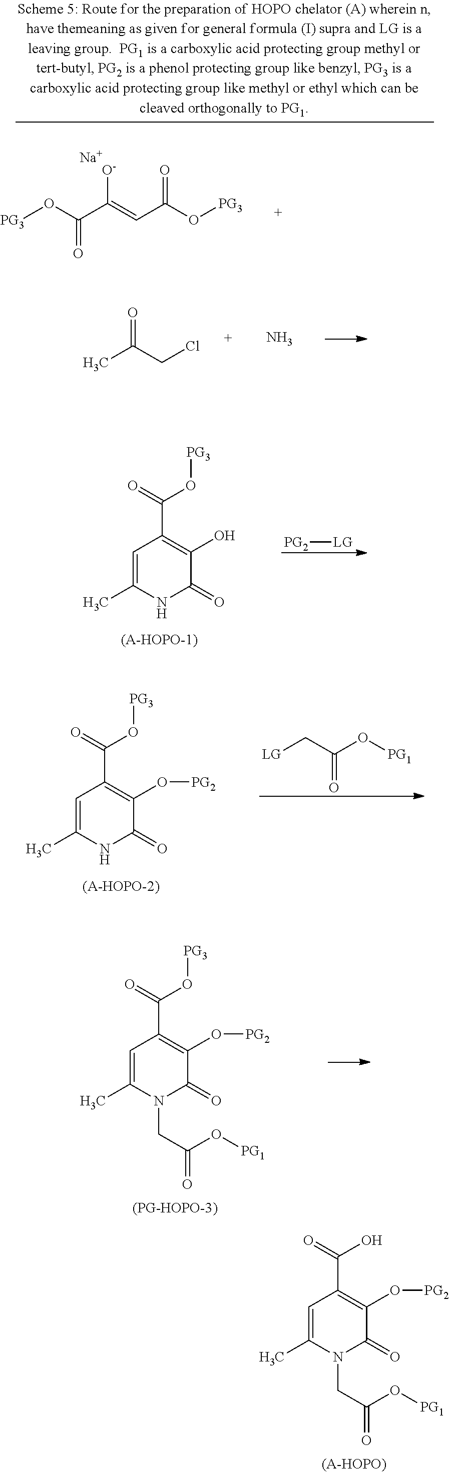

PG₃-protected oxalacetate sodium salt is reacted with chloroacetone and ammonia under suitable conditions to give protected hydroxypyridone A-HOPO-1. These reaction conditions include but are not limited to heating, elevated pressure or the use of a Lewis acid like aluminium trichloride. A-HOPO-1 is then protected at the phenol position by reaction with PG₂-X to give A-HOPO-2. After that A-HOPO-2 is reacted with an activated protected acetic acid equivalent like tert-butyl bromoacetate to give PG-HOPO-3. Finally PG₃ is cleaved selectively by conditions known to those skilled in the art like for example lithium hydroxide for the cleavage of ethyl or methyl esters to give A-HOPO.

The order of the second and third step in this synthesis can be exchanged meaning to alkylate the pyridine NH before protection of the phenol.

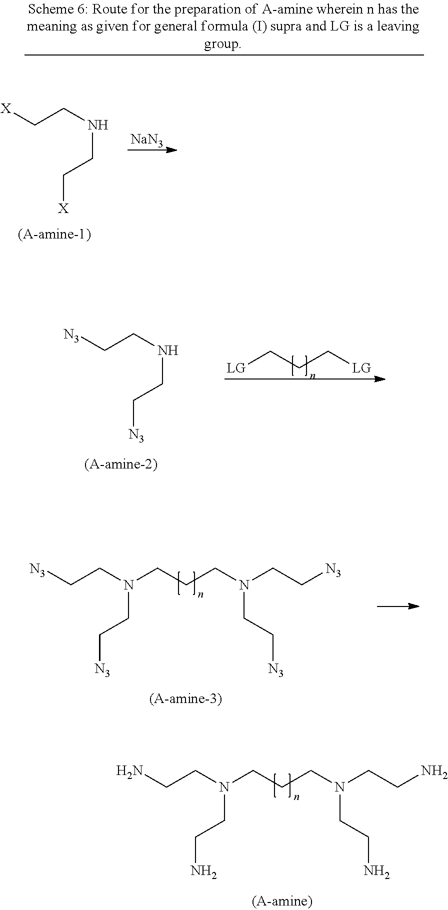

Bis-reactive A-amine-1 is reacted with an appropriate azide like sodium azide under conditions for alkylic nucleophilic displacement known to those skilled in the art to give bis azide A-amine-2. This is then further reacted with an appropriate bis-reactive alkane like 1,3-dibromopropoane under conditions for alkylic nucleophilic displacement known to those skilled in the art to give tetraazide A-amine-3. Tetraazide A-amine-3 is the reduced to tetraamien A-amine under conditions typical for the reduced of azides to the corresponding amine like catalytic hydrogenation with palladium on charcoal or with triphenyl phosphine.

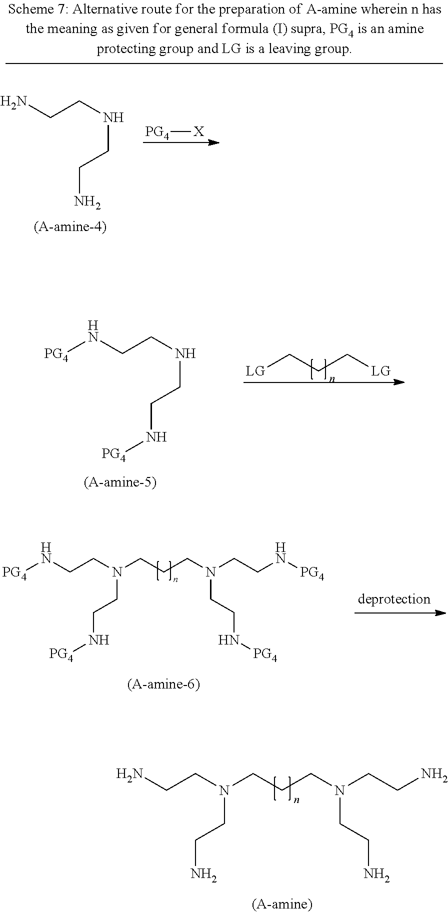

Trisamine A-amine-4 is protected at the terminal primary amines with a suitable protecting group like Boc, Fmoc, Cbz, or trityl to give bis-protected trisamine A-amine-5. This is then further reacted with an appropriate bis-reactive alkane like 1,3-dibromopropoane under conditions for alkylic nucleophilic displacement known to those skilled in the art to give tetrakis-protected hexaamine A-amine-6. A-amine-6 is then deprotected under conditions known to those skilled in the art to give A-amine.

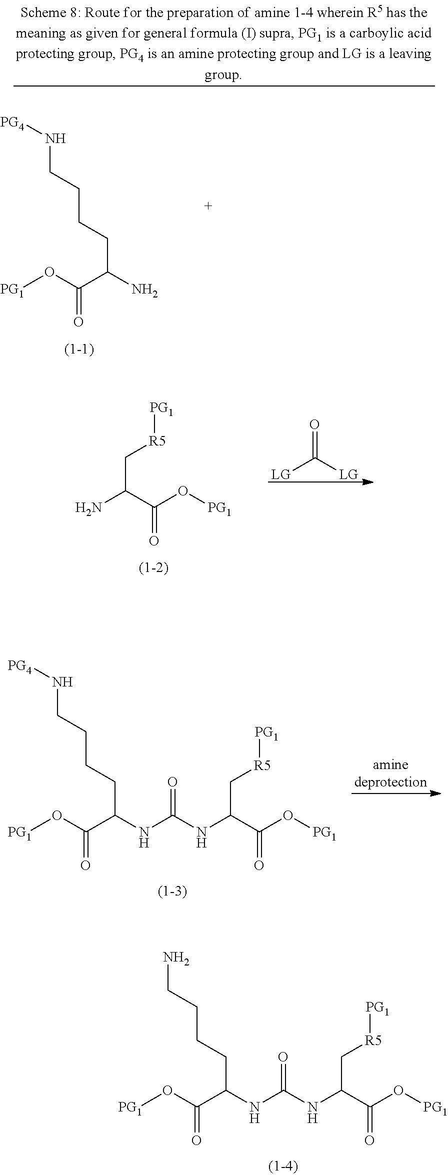

Protected lysine 1-1 is coupled with the α-amino group of protected amino acid 1-2 via an appropriate carbonic acid equivalent to give urea 1-3. This carbonic acid equivalent can be but is not limited to N,N′-carbonyl diimidazole, phosgene, diphosgene, triphosgene, or p-nitrophenylchloroformate and is reacted first with 1-1 and then treated with 1-2 together with a suitable base like for example N,N-diisopropylethylamine or triethylamine or reacted first with 1-2 and then treated with 1-1 together with a suitable base like for example N,N-diisopropylethylamine or triethylamine. In the next step 1-3 is deprotected at the ε-amino group of the lysine moiety under conditions known to those skilled in the art to give amine 1-4.

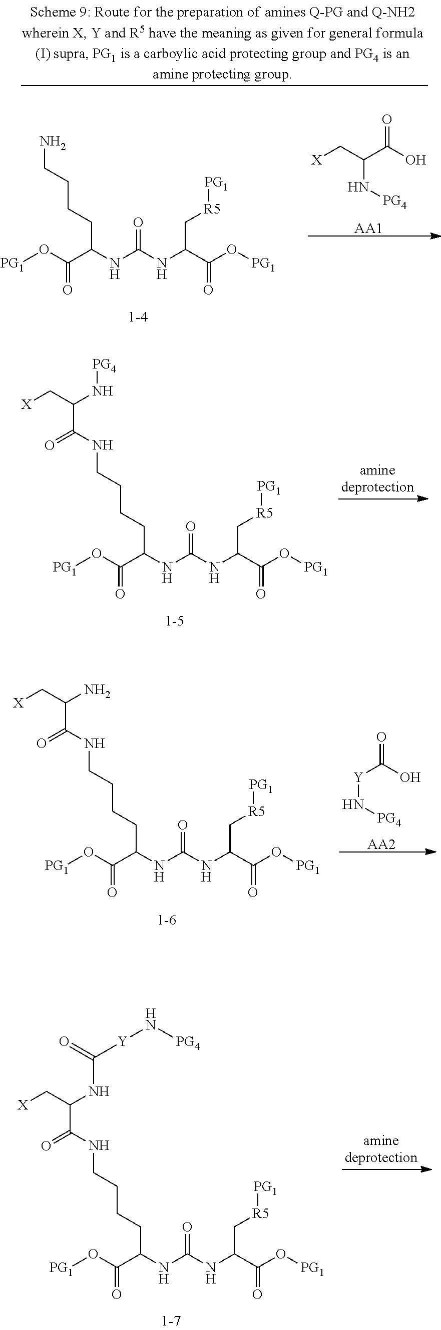

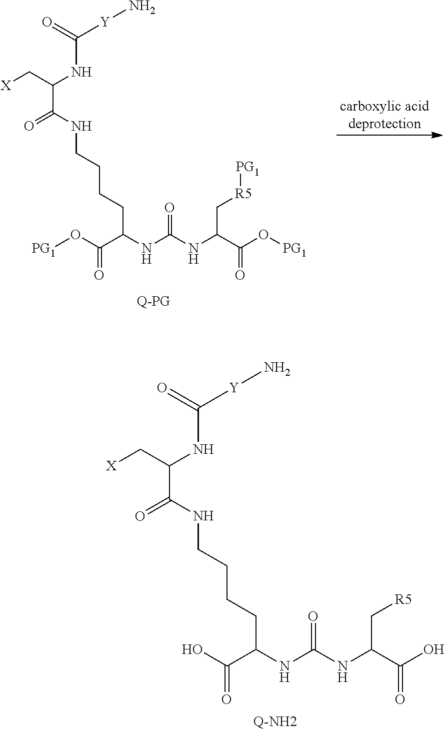

Amine 1-4 is coupled to N-protected amino acid AA1 by typical peptide coupling conditions known to those skilled in the art to give protected peptide 1-5. Typical reaction conditions include but are to limited to the use of coupling reagents like HATU, PyAOP, or DMTMM. 1-5 is then deprotected at the amine position by conditions known to those skilled in the art to give amine 1-6. These conditions include but are not limited to the use of trifluoroacetic acid for Boc protecting groups, catalytic hydrogenation for Cbz protecting groups or piperidine for Fmoc protecting groups. Amine 1-6 is then coupling with N-protected amino acid AA2 by typical peptide coupling conditions known to those skilled in the art to give protected peptide 1-7. 1-7 is then deprotected at the amine position by conditions known to those skilled in the art to give amine Q-PG. Q-PG is then preferably conjugated via amide bond formation using standard acid activating coupling reagents to the a carboxylate group on the HOPO chelators described in this invention. The use of stable tetrafluorophenol esters of compound (1) are preferred. Alternatively Q-PG can be deprotected at the carboxylic acid groups by conditions known to those skilled in the art to give Q-NH2. These conditions include but are not limited to trifluoro acetic acid or hydrochloric acid. The chelator moiety may then be coupled through amide bond formation using preformed active esters or other suitably activated carboxylate groups on the chelator.

These syntheses can also be performed on solid phase by conditions known to those skilled in the art by attaching amine AA2 or amine 1-4 to a solid phase like e.g. 2-chlorotrityl resin and cleaving it off the resin by suitable reagents like trifluoroacetic acid.

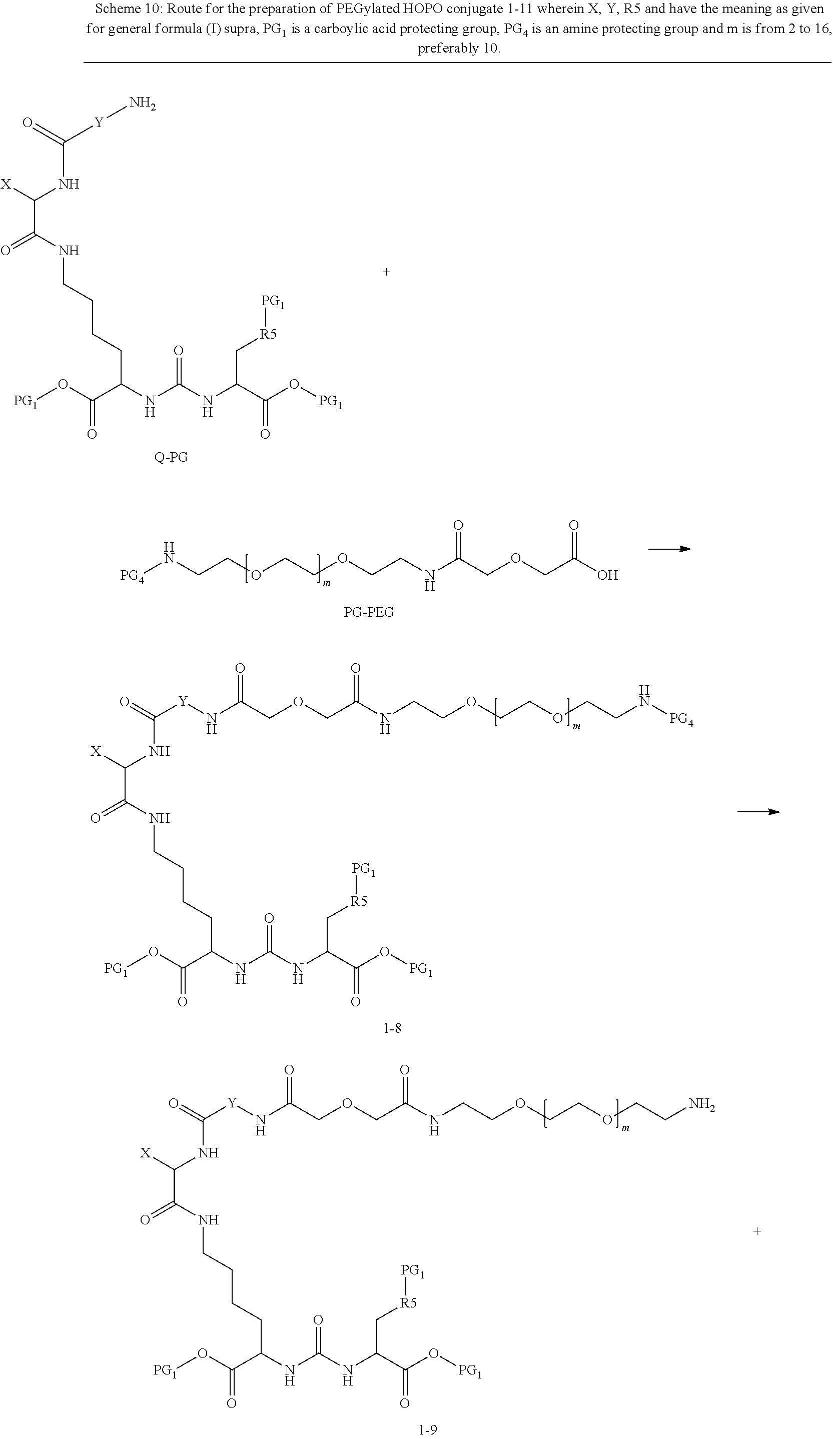

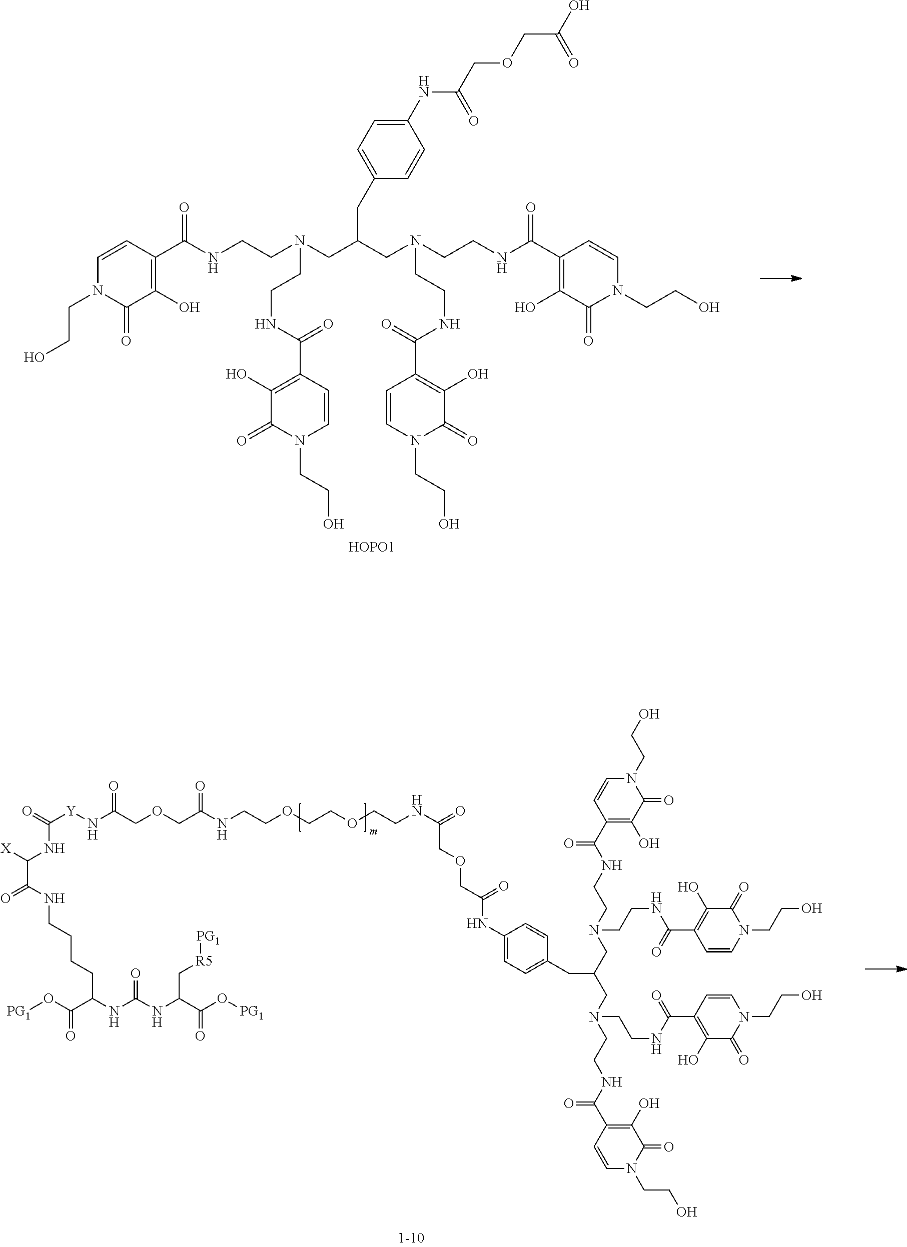

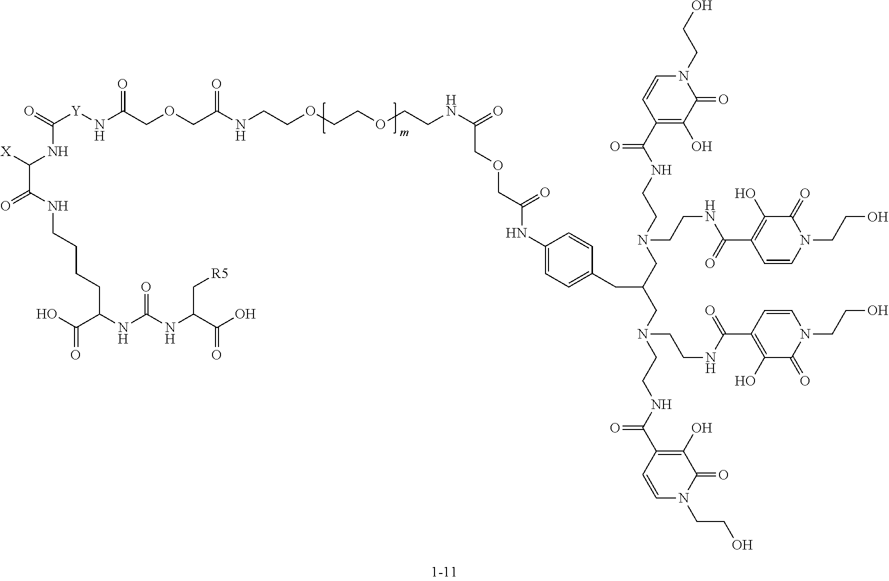

Q-PG is coupled to N-protected PEG carboxylic acid PG-PEG by standard amide coupling conditions known to those skilled in the art to give 1-8. These conditions include but are not limited to the use of coupling reagents like HATU or PyAOP. 1-8 is then deprotected at the amino group by conditions known to those skilled in the art to give amine 1-9. Amine 1-9 is the coupled to HOPO chelator glycolic acid HOPO1 by amide coupling conditions known to those skilled in the art to give 1-10. These conditions include but are not limited to the use of coupling reagents like HATU or PyAOP. HOPO1 can be synthesized by reacting the free aniline of HOPO1 (described in WO2013167756) with diglycolic acid anhydride with a suitable base like 2,4,6-trimethylpyridine. 1-10 is then deprotected at the carboxylic acid groups by conditions known to those skilled in the art to give conjugate 1-11. These conditions include but are not limited to trifluoro acetic acid or hydrochloric acid.

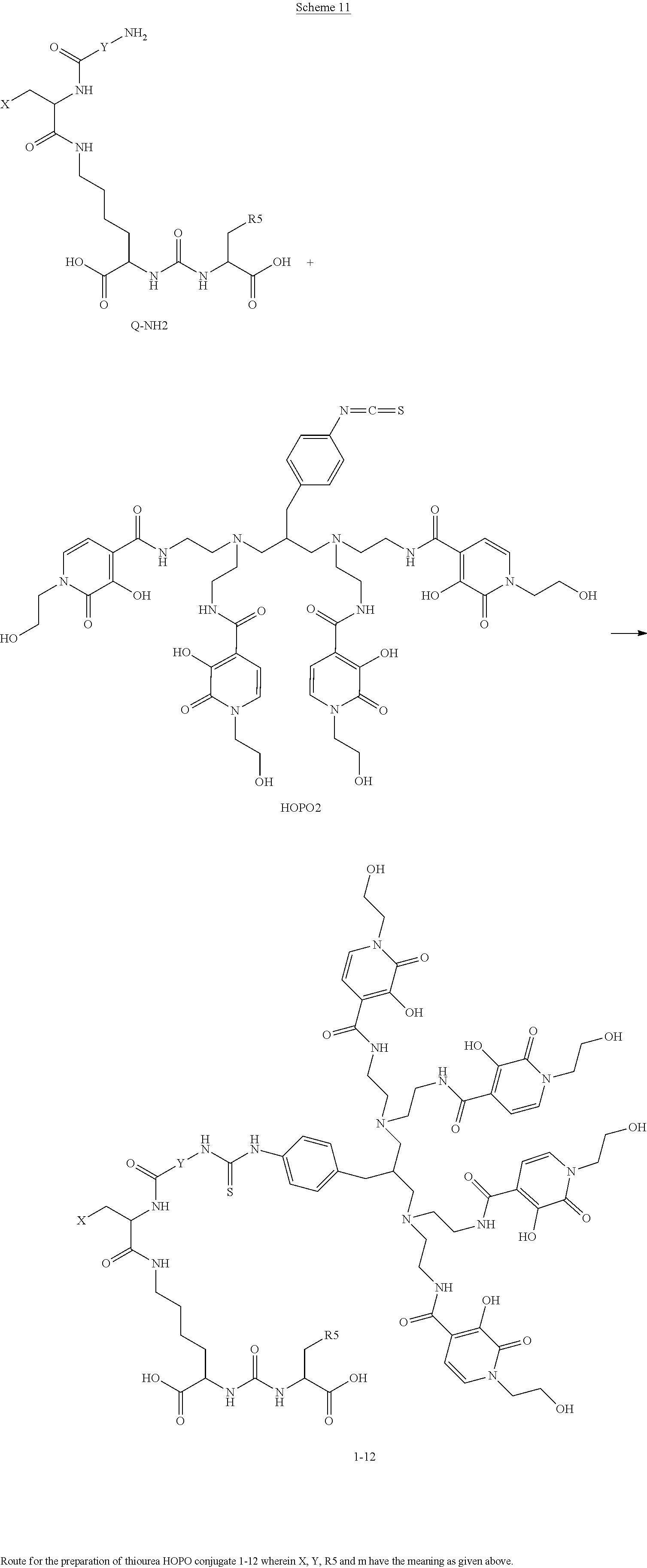

Amine Q-NH2 is reacted with HOPO isothiocyanate HOPO2 (described in WO 2013167756) in an appropriate buffer like borate buffer to give thiourea 1-12.

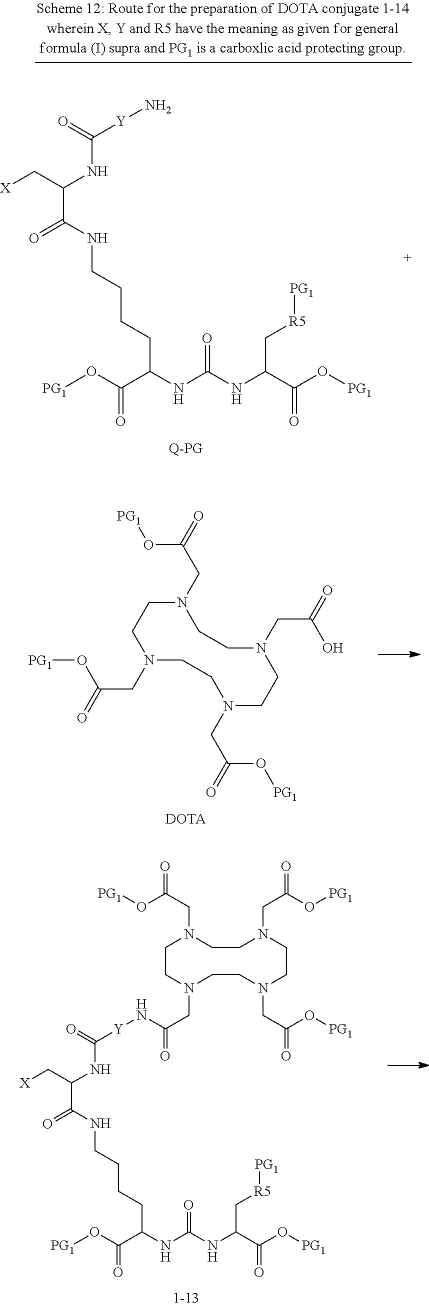

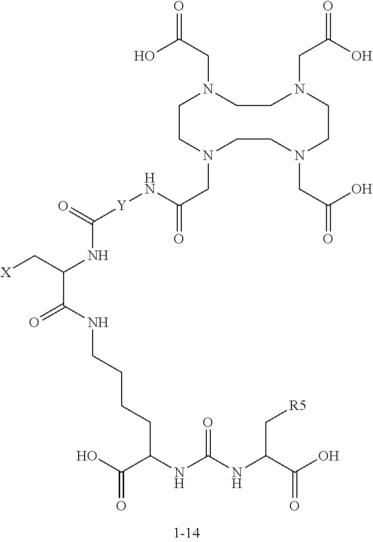

Amine Q-PG is coupled to tris-protected DOTA derivative (DOTA) by amide coupling conditions known to those skilled in the art to give 1-13. These conditions include but are not limited to the use of coupling reagents like HATU or PyAOP. 1-13 is then deprotected at the carboxylic acid groups by conditions known to those skilled in the art to give conjugate 1-14. These conditions include but are not limited to trifluoro acetic acid or hydrochloric acid.

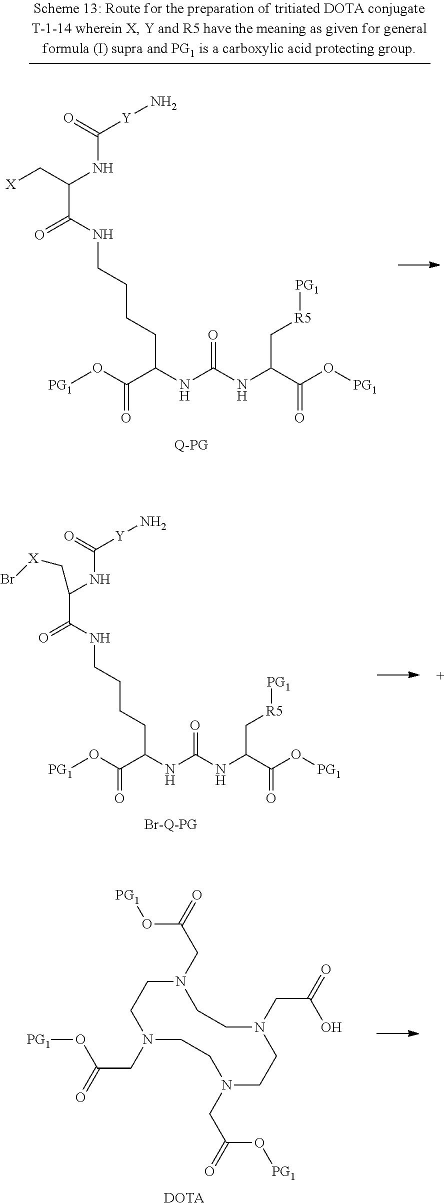

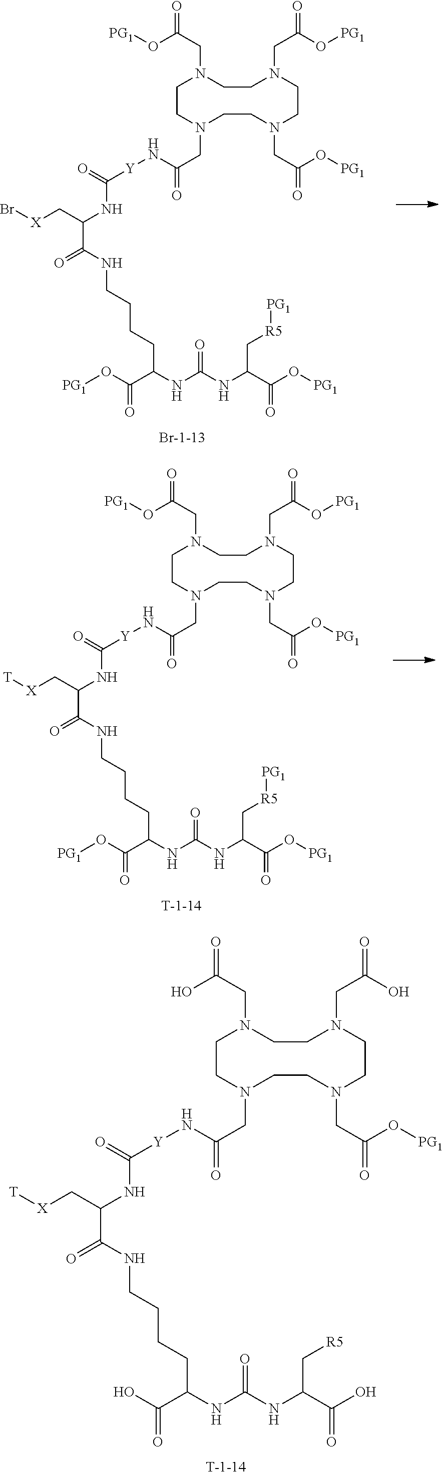

Amine Q-PG is brominated at residue X by conditions known to those skilled in the art to give Br-Q-PG. These conditions include but are not limited to the use of N-bromosuccinimide or bromine. Br-Q-PG is then coupled to tris-protected DOTA derivative (DOTA) by amide coupling conditions known to those skilled in the art to give Br-1-13. These conditions include but are not limited to the use of coupling reagents like HATU or PyAOP. Br-1-13 is then tritiated via catalytic tritiation to give T-1-13. T-1-13 is then deprotected at the carboxylic acid groups by conditions known to those skilled in the art to give conjugate T-1-14. These conditions include but are not limited to trifluoro acetic acid or hydrochloric acid.

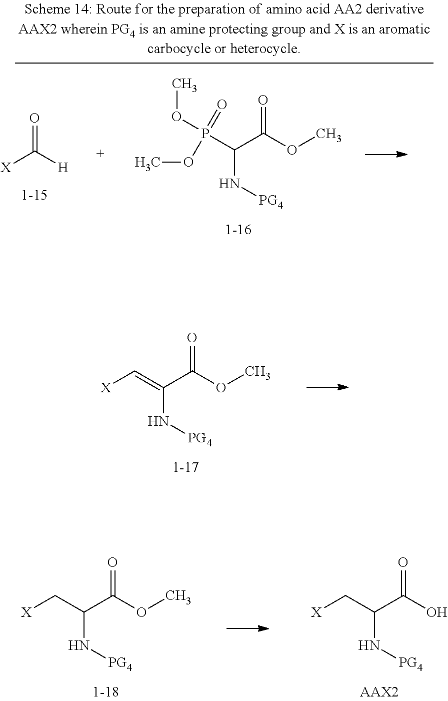

Amino acids of type AAX2 can be prepared according to R. Ramón et al., ChemBioChem 2011, 12, 625-632. Benzylic aldehyde 1-15 is reacted with phosphonate 1-16 in a Wittig-type reaction to give aminoacrylic ester 1-17. 1-17 is then reduced to protected amino acid 1-18. To yield the racemic amino acid 1-18 the reduction can be performed using achiral catalysts like palladium on charcoal under a hydrogen atmosphere. For stereoselective reduction yielding the enantiomerically pure or enriched amino acids 1-18 and AAX2 the reduction can be performed using a chiral catalyst like (R)—[Rh(COD)(MaxPhos)]BF₄ under a hydrogen atmosphere.

The benzylic aldehydes 1-15 can be prepared for example via oxidation of the corresponding benzyl alcohol using Dess-Martin periodinane or Swern conditions or by reduction of the corresponding benzoic acid using DIBAL-H. The corresponding benzyl alcohol can be synthesized by bromination of the methyl group of the corresponding toluoyl derivative followed by hydrolysis or substitution with acetate and subsequent ester hydrolysis or by reduction of the corresponding methyl ester with lithium aluminium hydride. Moreover 1-15 can be prepared by treating the corresponding arylbromide with n-butyllithium and N,N-dimethylformamide.

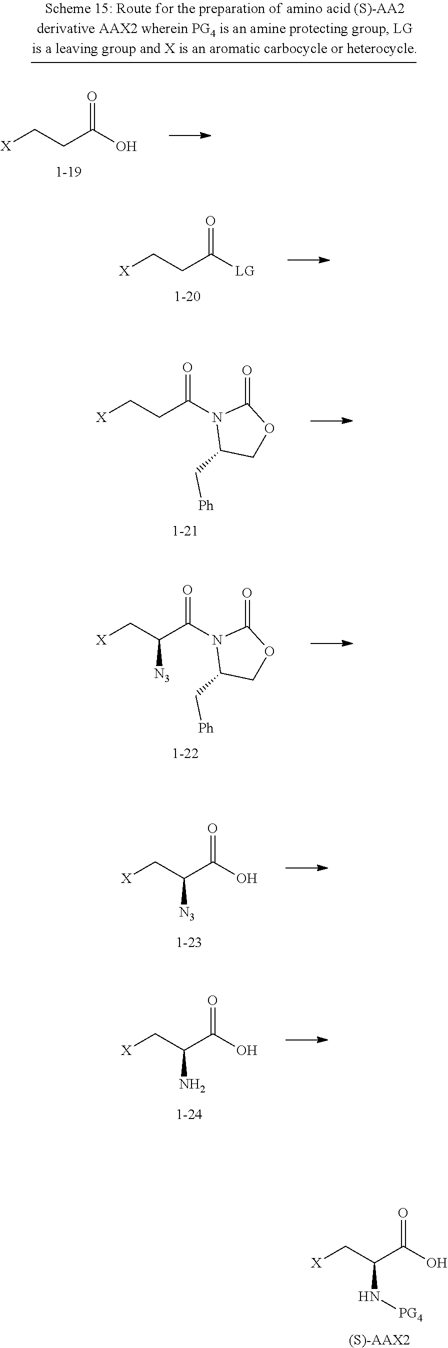

Another possibility to synthesize enantiomerically enriched or pure AAX2 is described in D. A. Evans et al., J. Am. Chem. Soc. 1987, 22, 6881 and starts with activation of the corresponding 3-aryl-propanoic acid 1-19 by for example oxalyl chloride or thionyl chloride to yield 1-20. This is then reacted with (4S)-4-benzyl-1,3-oxazolidin-2-one which was deprotonated with N-butyllithium to yield chiral imide 1-21. This is then deprotonated with a lithium bis(trimethylsilyl)amide and reacted with 2,4,6-tri(propan-2-yl)benzene-1-sulfonyl azide to give chirakl azido acid 1-23. Reduction of the α-azido group by triphenyl phosphine yield chiral amino acid 1-24 which is protected at the amino group to give (S)-AAX2.

### EXPERIMENTAL SECTION

Abbreviations

The following table lists the abbreviations used in this paragraph and in the Intermediates and Examples section as far as they are not explained within the text body.

TABLE 1
Abbreviations

| Abbreviation | Meaning |
|---|---|
| h | hour(s) |
| min | minute(s) |
| d | day(s) |
| aq. | aqueous |
| sat. | saturated |
| EtOAc | ethyl acetate |
| DCM | dichloromethane |
| MeOH | methanol |
| THF | tetrahydrofuran |
| DMF | N,N-dimethylformamide |
| DIPEA | N,N-diisopropylethylamine |
| HCI | hydrochloric acid |
| HATU | N-[(dimethylamino)(3H-[1,2,3]triazolo[4,5-b]pyridin-3- |
| | yloxy)methylidene]-N-methylmethanaminium |
| | hexafluorophosphate |
| HFIP | 1,1,1,3,3,3-Hexafluoro-2-propanol |
| T₃P | 2,4,6-tripropyl-1,3,5,2lambda⁵,4lambda⁵,6lambda⁵- |
| | trioxatriphosphinane-2,4,6-trione |
| SiO2 | silica |
| C18 | reversed phase |
| TIPS | Triisopropylsilane |
| TFA | Trifluroroacetic acid |
| HNO3 | Nitric acid |
| COMU | (1-Cyano-2-ethoxy-2- |
| | oxoethylidenaminooxy)dimethylamino-morpholino- |
| | carbenium hexafluorophosphate |
| (R)- | [((R)-tert-Butylmethylphosphino)(di-tert- |
| [Rh(COD) | butylphosphino)amine](1,5-cyclooctadiene)rhodium(I) |
| (MaxPHOS)]BF4 | tetrafluoroborate |
| PYAOP | (7-Azabenzotriazol-1-yloxy)tripyrrolidinophosphonium |
| | hexafluorophosphate |
| r.t. | Room temperature, ambient temperature (18-25° C.) |
| iTLC | Instant Thin Layer Chromatography |
| NMP | N-Methyl-2-pyrrolidone |
| tR | Time of retention |
| RAC | Radioactive concentration |
| TSTU | O-(N-Succinimidyl)-1,1,3,3-tetramethyluronium |
| | Tetrafluoroborate |
| ²²⁷Th | Thorium-227 |
| ACN | Acetonitrile |
| EDTA | ethylenediaminetetraacetic acid |
| HPLC | high performance liquid chromatography |
| pABA | para-aminobenzoic acid |
| PSMA | prostate-specific membrane antigen |
| Rₜ | time of retention |
| BnBr | Benzyl bromide |
| LC-MS | liquid chromatography-coupled mass spectrometry |
| MTBE | methyl tert-butyl ether |
| PE | Petroleum ether |
| 3,2-HOPO | 3-hydroxypyridin-2-one |
| 5T4 | oncofetal trophoblast glycoprotein |
| AlCl₃ | aluminum chloride |
| Amc | trans-4-(aminomethyl)cyclohexanecarboxylic acid |
| BL | blocked sample |
| BSA | bovine serum alumine |
| CAR | chelator-to-antibody ratio |
| CDCl₃ | deuterated chloroform |
| Ctrl | control mAb with 3,2-HOPO chelator |
| DMA | dimethylacetamide |
| DMSO | dimethylsulfoxide |
| DOTA | 1,4,7,10-tetraazacyclododecane-1,4,7,10-tetraacetic |
| | acid |
| ESI+ | electrospray ionization positive mode |
| EtOH | ethanol |
| FAP | fibroblast activation protein |
| FPLC | fast protein purification |
| g | gram |
| HER2 | human epidermal growth factor receptor 2 |
| HPGe | high purity germanium |
| IC50 | half maximal inhibitory concentration |
| IgG2A | immunoglobulin G subclass 2A |
| IRF | immunoreactive fraction |
| L | liter |
| LiOH | lithium hydroxide |
| mAbLY6G6D | monoclonal antibodylymphocyte antigen 6 family |
| | member G6F |
| m/zmAb | mass-to-charge ratiomonoclonal antibody |
| Mem/z | methylmass-to-charge ratio |
| MHzMe | megahertzmethyl |
| mLMHz | mililitermegahertz |
| mMmL | milimolarmililiter |
| MSmM | mass spectrometrymilimolar |
| Na₂SO₄MS | sodium sulfatemass spectrometry |
| NalNa₂SO₄ | napthylalaninesodium sulfate |
| NaOHNal | sodium hydroxidenapthylalanine |
| nmNaOH | nanometersodium hydroxide |
| NMRnm | nuclear magnetic resonancenanometer |
| nmolNMR | nanomolnuclear magnetic resonance |
| PBSnmol | phosphate buffered salinenanomol |
| PS80PBS | polysorbate 80phosphate buffered saline |
| RCPPS80 | radiochemical puritypolysorbate 80 |
| SECRCP | size-exclusion chromatographyradiochemical purity |
| TBSSEC | TRIS (tris(hydroxymethyl)aminomethane) buffered |
| | salinesize-exclusion chromatography |
| ThTBS | thoriumTRIS (tris(hydroxymethyl)aminomethane) |
| | buffered saline |
| UBLTh | unblocked samplethorium |
| ZrUBL | zirconiumunblocked sample |
| Zr | zirconium |

Amino acid Abbreviations

Ala=Alanine

Arg=Arginine

Asn=Asparagine

Asp=Aspartic acid

Cys=Cysteine

Glu=Glutamic acid

Gln=Glutamine

Gly=Glycine

His=Histidine

Ile=Isoleucine

Leu=Leucine

Lys=Lysine

Met=Methionine

Phe=Phenylalanine

Pro=Proline

Ser=Serine

Thr=Threonine

Trp=Tryptophan

Tyr=Tyrosine

Other abbreviations not specified herein have their meanings customary to the skilled person.

The various aspects of the invention described in this application are illustrated by the following examples which are not meant to limit in any way the full scope of the invention as described herein.

Specific Experimental Descriptions

NMR peak forms in the following specific experimental descriptions are stated as they appear in the spectra, possible higher order effects have not been considered. Chemical shifts (5) are given in ppm. The chemical shifts were corrected by setting the DMSO signal to 2.50 ppm unless otherwise stated.

The ¹H-NMR data of selected compounds are listed in the form of ¹H-NMR peaklists. Therein, for each signal peak the δ value in ppm is given, followed by the signal intensity, reported in round brackets. The δ value-signal intensity pairs from different peaks are separated by commas. Therefore, a peaklist is described by the general form: δ₁ (intensity₁), δ₂ (intensity₂), . . . , δᵢ (intensityᵢ), . . . , δₙ (intensityₙ).

The intensity of a sharp signal correlates with the height (in cm) of the signal in a printed NMR spectrum. When compared with other signals, this data can be correlated to the real ratios of the signal intensities. In the case of broad signals, more than one peak, or the center of the signal along with their relative intensity, compared to the most intense signal displayed in the spectrum, are shown. A ¹H-NMR peaklist is similar to a classical ¹H-NMR readout, and thus usually contains all the peaks listed in a classical NMR interpretation. Moreover, similar to classical ¹H-NMR printouts, peaklists can show solvent signals, signals derived from stereoisomers of the particular target compound, peaks of impurities, ¹³C satellite peaks, and/or spinning sidebands. The peaks of stereoisomers, and/or peaks of impurities are typically displayed with a lower intensity compared to the peaks of the target compound (e.g., with a purity of >90%). Such stereoisomers and/or impurities may be typical for the particular manufacturing process, and therefore their peaks may help to identify a reproduction of the manufacturing process on the basis of “by-product fingerprints”. An expert who calculates the peaks of the target compound by known methods (MestReC, ACD simulation, or by use of empirically evaluated expectation values), can isolate the peaks of the target compound as required, optionally using additional intensity filters. Such an operation would be similar to peak-picking in classical ¹H-NMR interpretation. A detailed description of the reporting of NMR data in the form of peaklists can be found in the publication “Citation of NMR Peaklist Data within Patent Applications” (cf. http://www.researchdisclosure.com/searching-disclosures, Research Disclosure Database Number 605005, 2014, 1 Aug. 2014). In the peak picking routine, as described in the Research Disclosure Database Number 605005, the parameter “MinimumHeight” can be adjusted between 1% and 4%. However, depending on the chemical structure and/or depending on the concentration of the measured compound it may be reasonable to set the parameter “MinimumHeight”<1%.

Reactions employing microwave irradiation may be run with a Biotage Initiator® microwave oven optionally equipped with a robotic unit. The reported reaction times employing microwave heating are intended to be understood as fixed reaction times after reaching the indicated reaction temperature. The compounds and intermediates produced according to the methods of the invention may require purification. Purification of organic compounds is well known to the person skilled in the art and there may be several ways of purifying the same compound. In some cases, no purification may be necessary. In some cases, the compounds may be purified by crystallization. In some cases, impurities may be stirred out using a suitable solvent. In some cases, the compounds may be purified by chromatography, particularly flash column chromatography, using for example prepacked silica gel cartridges, e.g. from Separtis such as Isolute® Flash silica gel (“SiO₂”) or Isolute® Flash NH₂ silica gel (“amine-coated SiO₂”) in combination with a Isolera autopurifier (Biotage) and eluents such as gradients of e.g. hexane/ethyl acetate or dichloromethane/methanol. In some cases, the compounds may be purified by preparative HPLC using for example a Waters autopurifier equipped with a diode array detector and/or on-line electrospray ionization mass spectrometer in combination with a suitable prepacked reverse phase column and eluents such as gradients of water and acetonitrile which may contain additives such as trifluoroacetic acid, formic acid or aqueous ammonia. In some cases, purification methods as described above can provide those compounds of the present invention which possess a sufficiently basic or acidic functionality in the form of a salt, such as, in the case of a compound of the present invention which is sufficiently basic, a trifluoroacetate or formate salt for example, or, in the case of a compound of the present invention which is sufficiently acidic, an ammonium salt for example. A salt of this type can either be transformed into its free base or free acid form, respectively, by various methods known to the person skilled in the art, or be used as salts in subsequent biological assays. It is to be understood that the specific form (e.g. salt, free base etc) of a compound of the present invention as isolated as described herein is not necessarily the only form in which said compound can be applied to a biological assay in order to quantify the specific biological activity.

The purities of the intermediates and examples are >95% as judged by LC/MS or ¹H-NMR spectra if not stated otherwise.

The percentage yields reported in the following examples are based on the starting component that was used in the lowest molar amount. Air and moisture sensitive liquids and solutions were transferred via syringe or cannula, and introduced into reaction vessels through rubber septa. Commercial grade reagents and solvents were used without further purification. The term “concentrated under reduced pressure” refers to use of a Buchi rotary evaporator at a minimum pressure of approximately 15 mm of Hg. All temperatures are reported uncorrected in degrees Celsius (° C.).

In order that this invention may be better understood, the following examples are set forth. These examples are for the purpose of illustration only, and are not to be construed as limiting the scope of the invention in any manner. All publications mentioned herein are incorporated by reference in their entirety.

Generation of ²²⁷Th

²²⁷Th was selectively isolated from an ²²⁷Ac mixture, which had been growing in daughters for two weeks, by adding 0.25 ml of 7M HNO₃ to the actinium mixture (which had been evaporated to dryness) and eluting the solution through an anion exchange column. The column had an inner diameter of 2 mm and a length of 30 mm containing approximately 70 mg of AG-1×8 anion exchange resin (Biorad Laboratories, Hercules, Calif., USA) (nitrate form). The column was washed with 2-4 ml of 7M HNO₃ to remove ²²⁷Ac, ²²³Ra and Ra daughters while retaining ²²⁷Th. Subsequently ²²⁷Th was stripped from the column with a few ml of 12M HCl. Finally the HCl was evaporated to dryness and the ²²⁷Th re-dissolved in 0.05 M HCl.

Antibodies

The FAP-targeting antibody was prepared according to WO2017/211809.

The PSMA-targeting antibody is BAY 2315497 and is prepared according to Example 9, specifically Examples 9a and 9b of WO 2016/096843.

The HER2-targeting antibody is prepared according to Example 5, specifically Examples 5a and 5b of WO 2016/096843.

Analytical LC-MS and HPLC Conditions

LC-MS-data given in the subsequent specific experimental descriptions refer (unless otherwise noted) to the following conditions:

Method 1:

Instrument: Waters Acquity UPLCMS SingleQuad; Column: Acquity UPLC BEH C18 1.7 μm, 50×2.1 mm; eluent A: water+0.1 vol % formic acid (99%), eluent B: acetonitrile; gradient: 0-1.6 min 1-99% B, 1.6-2.0 min 99% B; flow 0.8 ml/min; temperature: 60° C.; DAD scan: 210-400 nm.

Method 2:

Instrument: Waters Acquity UPLCMS SingleQuad; Column: Acquity UPLC BEH C18 1.7 μm, 50×2.1 mm; eluent A: water+0.2 vol % aqueous ammonia (32%), eluent B: acetonitrile; gradient: 0-1.6 min 1-99% B, 1.6-2.0 min 99% B; flow 0.8 ml/min; temperature: 60° C.; DAD scan: 210-400 nm.

Method 3:

Instrument: Waters Acquity UPLCMS SingleQuad; Column: Acquity UPLC BEH C18 1.7 μm, 50×2.1 mm; eluent A: water+0.1 vol-% formic acid (99%), eluent B: acetonitrile; gradient: 0-1.7 min 1-45% B, 1.7-1.72 min 45-99% B, 1.72-2.0 min 99% B; flow 0.8 ml/min; temperature: 60° C.; ELSD.

Method A:

Instrument: SHIMADZU LCMS-2020 SingleQuad; Column: Chromolith@Flash RP-18E 25-2 MM; eluent A: water+0.0375 vol % trifluoroacetic acid, eluent B: acetonitrile+0.01875 vol % trifluoroacetic acid; gradient: 0-0.8 min 0-60% B, 0.8-1.2 min 60% B; flow 1.5 ml/min; temperature: 50° C.; PDA: 220 nm & 254 nm.

Method B:

Instrument: Agilent 1100\G1956A SingleQuad; Column: Kinetex@ 5 um EVO C18 30*2.1 mm; eluent A: water+0.0375 vol % trifluoroacetic acid, eluent B: acetonitrile+0.01875 vol % trifluoroacetic acid; gradient: 0-0.8 min 0-60% B, 0.8-1.2 min 60% B; flow 1.5 ml/min; temperature: 50° C.; PDA: 220 nm & 254 nm.

Method C:

Instrument: SHIMADZU LCMS-2020 SingleQuad; Column: Chromolith@Flash RP-18E 25-2 MM; eluent A: water+0.0375 vol % trifluoroacetic acid, eluent B: acetonitrile+0.01875 vol % trifluoroacetic acid; gradient: 0-0.8 min, 5-95% B, 0.8-1.2 min 95% B; flow 1.5 ml/min; temperature: 50° C.; PDA: 220 nm & 254 nm.

Method D:

Instrument: Agilent 1100\G1956A SingleQuad; Column: Kinetex@ 5 μm EVO C18 30*2.1 mm; eluent A: water+0.0375 vol % trifluoroacetic acid, eluent B: acetonitrile+0.01875 vol % trifluoroacetic acid; gradient: 0-0.8 min 5-95% B, 0.8-1.2 min 95% B; flow 1.5 ml/min; temperature: 50° C.; PDA: 220 nm & 254 nm.

Method E:

Instrument: Agilent 1200\G6110A SingleQuad; Column: XBridge C18 2.1*50 mm, 5 μm; eluent A: water+0.025 vol % ammonium hydroxide, eluent B: acetonitrile; gradient: 0-1.2 min 10-80% B, 1.2-1.6 min 80% B; flow 1.2 ml/min; temperature: 40° C.; DAD: 220 nm & 254 nm.

Method F:

Instrument: Agilent 1200\G6110A SingleQuad; Column: XBridge C18 2.1*50 mm, 5 μm; eluent A: water+0.025 vol % ammonium hydroxide, eluent B: acetonitrile; gradient: 0-1.2 min 0-60% B, 1.2-1.6 min 60% B; flow 1.0 ml/min; temperature: 40° C.; DAD: 220 nm & 254 nm.

Method G:

Instrument: SHIMADZU LCMS-2020 SingleQuad; Column: Kinetex EVO C18 2.1*30 mm, 5 μm; eluent A: water+0.025 vol % ammonium hydroxide, eluent B: acetonitrile; gradient: 0-0.8 min, 5-95% B, 0.8-1.2 min 95% B; flow 1.5 ml/min; temperature: 40° C.; PDA: 220 nm & 254 nm.

Method H:

Instrument: SHIMADZU LCMS-2020 SingleQuad; Column: Kinetex EVO C18 2.1*30 mm, 5 μm; eluent A: water+0.025 vol % ammonium hydroxide, eluent B: acetonitrile; gradient: 0-1.2 min, 0-60% B, 1.2-1.6 min, 60% B; flow 1.0 ml/min; temperature: 40° C.; PDA: 220 nm & 254 nm.

Method I:

Instrument: SHIMADZU LCMS-2020 SingleQuad; Column: Kinetex EVO C18 2.1*30 mm, 5 um; eluent A: water+0.025 vol % ammonium hydroxide, eluent B: acetonitrile; gradient: 0-0.8 min, 5-95% B, 0.8-1.2 min, 95% B; flow 1.5 ml/min; temperature: 40° C.; PDA: 220 nm & 254 nm.

Method J:

Instrument: SHIMADZU LCMS-2020 SingleQuad; Column: Kinetex EVO C18 2.1*30 mm, 5 μm; eluent A: water+0.025 vol % ammonium hydroxide, eluent B: acetonitrile; gradient: 0-0.8 min, 0-60% B, 0.8-1.2 min, 60% B; flow 1.5 ml/min; temperature: 40° C.; PDA: 220 nm & 254 nm.

Method K:

Instrument: Waters Acquity/QTOF; Column: Phenomenex Kinetex 1.7 μm C18, 100 Å, 30×2.1 mm; eluent A: Water/0.1% TFA; eluent B: ACN/0.1% TFA; flow: 0.5 mL/min; temperature: ambient; Detection: PDA

Method L:

Column: Agilent Kinetex EVO C18 30*2.1 mm, 5 um. MPA: 0.0375% TFA in water. MPB: 0.0188% TFA in acetonitrile. Gradient: 0.00 min 5% B→0.80 min 95% B→1.20 min 95% B→1.21 min 5% B→1.50 min 5% B; flow rate: 1.5 mL/min; Column Temperature: 50° C.; UV detection: 220 nm & 254 nm.

Method M

Equipment type MS: Waters TOF instrument; Equipment type UPLC: Waters Acquity I-CLASS; Column: Waters, HSST3, 2.1×50 mm, C18 1.8 μm; eluent A: 1 L water+0.01% formic acid; eluent B: 1 L acetonitrile+0.01% formic acid; gradient: 0.0 min 2% B→0.5 min 2% B→7.5 min 95% B→10.0 min 95% B; oven: 50° C.; flow rate: 1.00 mL/min; UV-detection: 210 nm

Method N

Equipment type MS: ThermoFisherScientific LTQ-Orbitrap-XL; Equipment type HPLC: Agilent 1200SL; Column: Agilent, POROSHELL 120, 3×150 mm, SB—C18 2.7 μm; eluent A: 1 L water+0.1% trifluoroacetic acid; eluent B: 1 L acetonitrile+0.1% trifluoroacetic acid; gradient: 0.0 min 2% B→1.5 min 2% B→15.5 min 98% B→18.0 min 98% B; oven: 40° C.; flow rate: 0.75 mL/min; UV-detection: 210 nm

Methode o (Preparative RP-Chromatography)

Instrument: Knauer Blue Shadow (Pump 40P, Azura Detector UVD 2.1S) with Merck/Hitachi D-2500 Chromato-Integrator; Column: Nucleodur C18 5 μm Gravity, 250×21 mm; eluent A: acetonitrile, eluent B: water+0.2% trifluoroacetic acid; gradient: 0-25 min 55% A, 25-30 min 55-90% A, 30-45 min 90% A; flow 5.0 mL/min; temperature: r.t.; UV scan: 226 nm.

Methode P

Instrument: Agilent 6550 Series Funnel Q-TOF; Instrument HPLC: Agilent 1290 Infinity Series; Column: Kinetix C18 1.7 μm, 100×1.7 mm; eluent A: water+0.1 vol % formic acid (99%), eluent B: acetonitrile+0.1 vol % formic acid (99%); gradient: 0-2 min 5% B, 2.0-3.0 min 5-90% B; flow 1.0 mL/min; temperature: 40° C.; UV scan: 230 nm.

Methode Q (Analytical RP-Chromatography)

Instrument: Agilent HP 1200 (UV) and Raytest Ramona® 2000 (radioactivity signal), the radioactivity signal was digitized by Agilent A/D-converter 35900E and evaluated by the work station; Column: Aquasil C18 5 μm 125 A, 150×4.6 mm; eluent A: acetonitrile, eluent B: water+0.2% trifluoroacetic acid; gradient: 0-30 min 10-90% A, 30-40 min 90% A; flow 1.3 mL/min; temperature: r.t.; UV scan: 254 nm.

Methode R (Preparative RP-Chromatography)

Instrument: Knauer Blue Shadow (Pump 40P, Azura Detector UVD 2.1S) with Merck/Hitachi D-2500 Chromato-Integrator; Column: Nucleodur C18 5 μm Gravity, 250×21 mm; eluent A: acetonitrile, eluent B: water+0.2 vol % trifluoroacetic acid; gradient: 0-30 min 10-50% A, 30-35 min 50-90% A, 35-40 min 90% A; flow 5.0 mL/min; temperature: r.t.; UV scan: 226 nm.

Methode S

Instrument: Agilent 6200 Series TOF; Instrument HPLC: Agilent 1290 Infinity Series; Column: Kinetix C18 1.7 μm, 100×1.7 mm; eluent A: water+0.1 vol % formic acid (99%), eluent B: acetonitrile+0.1 vol % formic acid (99%); gradient: 0-2 min 5% B, 2.0-3.0 min 5-90% B; flow 1.0 mL/min; temperature: 40° C.; UV scan: 210 nm.

Methode T (Analytical RP-Chromatography)

Instrument: Agilent HP 1260 Infinity (UV) and Raytest Ramona Star® (radioactivity signal), the radioactivity signal was digitized by Agilent A/D-converter 35900E and evaluated by the work station; Column: Aquasil C18 5 μm 125 A, 150×4.6 mm; eluent A: acetonitrile, eluent B: water+0.2% trifluoroacetic acid; gradient: 0-30 min 10-90% A, 30-40 min 90% A; flow 1.3 mL/min; temperature: r.t.; UV scan: 254 nm.

Flash Column Chromatography Conditions

“Purification by (flash) column chromatography” as stated in the subsequent specific experimental descriptions refers to the use of a Biotage Isolera purification system. For technical specifications see “Biotage product catalogue” on www.biotage.com.

Synthetic Procedures

Carboxy-HOPO Chelator

Intermediate a

### Ethyl 3-hydroxy-6-methyl-2-oxo-1,2-dihydropyridine-4-carboxylate

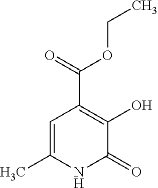

To a solution of sodium diethyloxylacetate (42.1 g, 200 mmol) in dry THF (500 mL) in a 1-liter 3-neck round bottom flask was added chloroacetone (16 mL, 200 mmol). After 10 min NH3 gas was bubbled trough the reaction followed by careful addition of AlCl3 (2.67 g, 20 mmol). The reaction was stirred under ambient temperature for 5 days. The resulting orange solid was filtered and taken up in 1 M HCl (500 mL) so that pH was below 3. The resulting suspension was stirred for 30 min and the precipitate filtered, washed with water and dried to give 25.5 g (65%) of the target compound as a pale yellow solid.

Intermediate b

### 3-(benzyloxy)-6-methyl-2-oxo-1,2-dihydropyridine-4-carboxylate

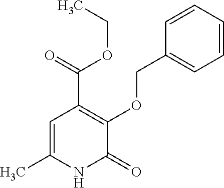 [SMILES: CCOC(=O)c1cc(C)[nH]c(=O)c1OCc1ccccc1]

1,8-Diazabicyclo[5.4.0]undec-7-ene (30.0 g, 197 mmol) was added to a solution of ethyl 3-hydroxy-6-methyl-2-oxo-1,2-dihydropyridine-4-carboxylate (25.5 g, 129 mmol) in isopropanol (300 mL). The reaction mixture was refluxed at 83° C. under N₂ before adding benzyl bromide (24 mL, 202 mmol) slowly. Refluxing was maintained for 4 hours, and then the solvent was evaporated. The resulting dark brown oil was dissolved in dichloromethane (100 mL), washed with aqueous 3 M HCl (2×100 mL) and water (3×100 mL). The organic layer was dried (Na2SO4), filtered and concentrated. Diisopropyl ether (500 mL) was added to the oily residue and the after spinning on the rotary evaporator the solid was filtered and dried to give 25 grams (67%) of the target compound. as a light grey solid.

Intermediate c

### Ethyl 3-(benzyloxy)-1-(2-(tert-butoxy)-2-oxoethyl)-6-methyl-2-oxo-1,2-dihydropyridine-4-carboxylate

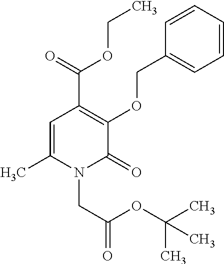 [SMILES: CCOC(=O)c1cc(C)n(CC(=O)OC(C)(C)C)c(=O)c1OCc1ccccc1]

To a solution of tert-butylbromoacetate (25.0 g, 0.128 mol) in acetone (130 mL) was added sodium iodide (25.6 g, 0.17 mol). The reaction mixture was heated at reflux for 9 hours, cooled to room temperature, filtered and concentrated. The crude product was used without further purification.

To a 1 L round bottom flask was added 3-(benzyloxy)-6-methyl-2-oxo-1,2-dihydropyridine-4-carboxylate (20.9 g, 72.8 mmol) followed by potassium fluoride on alumina (40 g). These reagents were purged under nitrogen for 15 minutes after which dimethoxyethane (320 mL) was added. To this solution was added tert-butyliodoacetate (35 g, 144 mmol) and the reaction was left to stir overnight, filtered and the filter cake washed thoroughly with THF. The filtrate was concentrated and the residue purified by flash chromatography (heptane-heptane/EtOAc 90:10-80:20-70:30-50:50) to afford 25.5 gram (87%) of the target compound.

Intermediate d

### 3-(Benzyloxy)-1-(2-(tert-butoxy)-2-oxoethyl)-6-methyl-2-oxo-1,2-dihydropyridine-4-carboxylic acid

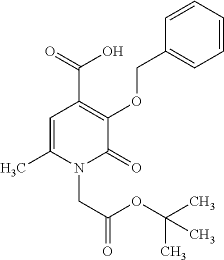 [SMILES: Cc1cc(C(=O)O)c(OCc2ccccc2)c(=O)n1CC(=O)OC(C)(C)C]

To a solution of ethyl 3-(benzyloxy)-1-(2-(tert-butoxy)-2-oxoethyl)-6-methyl-2-oxo-1,2-dihydropyridine-4-carboxylate (25.5 g, 63.5 mmol) in 1:1 THF/H2O (500 mL) was added aqueous LiOH (76 mL 1M, 76.2 mmol) and the reaction mixture stirred overnight at ambient temperature. Aqueous citric acid was added to neutral pH and the aqueous phase was extracted with EtOAc (×3). The combined organic extract was dried (Na2SO4), filtered and concentrated. The residue was triturated with heptane/EtOAc 90:10 and the precipitated material filtered and dried to afford 21.9 g (92%) of the target compound as a colorless solid.

MS (ESIpos): m/z=374.1 [M+H]⁺

Intermediate e

### Bis(2-azidoethyl)amine

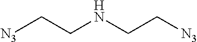 [SMILES: [N-]=[N+]=NCCNCCN=[N+]=[N-]]

To a stirred solution of sodium azide (56.0 g, 0.86 mol) in water (500 mL) was added bis(2-chloroethyl)amine hydrochloride (76.8 g, 0.43 mol). After stirring for 2 hours at 90° C., another portion of sodium azide (56.0 g, 0.86 g) was added, and the reaction mixture stirred for 48 hours at 90° C. After cooling to room temperature, the pH was adjusted to around 10 with aqueous NaOH (10 M). The aqueous solution was extracted with diethyl ether (×5). The combined organic extract was dried (Na2SO4), filtered and concentrated. Purification by chromatography (heptane/EtOAc gradient) afforded 45.5 g (68%) of the target compound as a yellow oil.

Intermediate f

### N1,N1,N3,N3-tetrakis(2-azidoethyl)propane-1,3-diamine

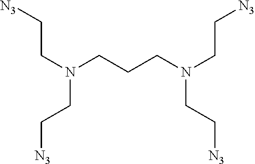 [SMILES: [N-]=[N+]=NCCN(CCCN(CCN=[N+]=[N-])CCN=[N+]=[N-])CCN=[N+]=[N-]]

To a solution of 1,3-dibromopropane (15 mL, 147 mmol), potassium carbonate (101 g, 730 mmol) and potassium iodide (48 g, 280 mmol) in acetonitrile (500 mL) was added slowly bis(2-azidoethyl)amine dissolved in acetonitrile and the reaction mixture heated at 80° C. for 48 hours. The solid was removed by filtration and the filtrate concentrated in vacuo. The residue was purified by column chromatography using heptane/EtOAc (0-50% EtOAc) to afford 38 grams (74%) of the target compound as a yellow oil.

Intermediate q

### N1,N1′-(propane-1,3-diyl)bis(N1-(2-aminoethyl)ethane-1,2-diamine)

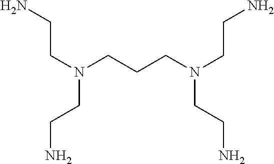 [SMILES: NCCN(CCN)CCCN(CCN)CCN]

A solution of N1,N1,N3,N3-tetrakis(2-azidoethyl)propane-1,3-diamine (5.20 g) in EtOH (100 mL) was hydrogenated overnight at 5 bar in the presence of Pd (2 g). Filtration and concentration afforded the target compound as a yellow oil used without further purification.

MS (ESIpos): m/z=247.2 [M+H]⁺

Intermediate h

### Tetra-tert-butyl 2,2′,2″,2′″-((5,9-bis(2-formamidoethyl)-2,5,9,12-tetraazatridecanedioyl)-tetrakis-(3-(benzyloxy)-6-methyl-2-oxopyridine-4,1(2H)-diyl))tetraacetate

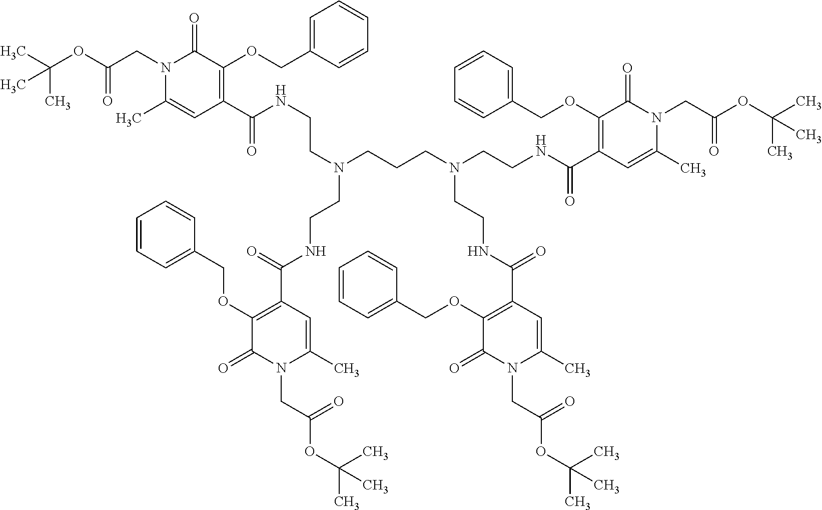 [SMILES: Cc1cc(C(=O)NCCN(CCCN(CCNC(=O)c2cc(C)n(CC(=O)OC(C)(C)C)c(=O)c2OCc2ccccc2)CCNC(=O)c2cc(C)n(CC(=O)OC(C)(C)C)c(=O)c2OCc2ccccc2)CCNC(=O)c2cc(C)n(CC(=O)OC(C)(C)C)c(=O)c2OCc2ccccc2)c(OCc2ccccc2)c(=O)n1CC(=O)OC(C)(C)C]

To a solution of N1,N1′-(propane-1,3-diyl)bis(N1-(2-aminoethyl)ethane-1,2-diamine) (246 mg, 1 mmol) and 3-(benzyloxy)-1-(2-(tert-butoxy)-2-oxoethyl)-6-methyl-2-oxo-1,2-dihydropyridine-4-carboxylic acid (1.50 g, 4 mmol) in DMF (15 mL) was added DIPEA (2 mL, 12 mmol), followed by HATU (3.0 g, 8 mmol). The reaction mixture was stirred at room temperature overnight, poured into water and extracted three times with EtOAc. The combined organic extract was washed with brine, dried (Na₂SO₄), filtered and concentrated. Flash chromatography (DCM/EtOAc 50:50-DCM/EtOAc/MeOH 47.5:47.5:5-DCM/EtOAc/MeOH 45:45:10) afforded 0.45 g (27%) of the target compound as a yellow solid.

Different batches were combined and purified by flash chromatography to afford 2.70 g of the target compound as a yellow solid used in the final step.

LC-MS (Method K, gradient: 10-70% B over 3 min): Rₜ=3.01 min; MS (ESIpos): m/z=1667.7 [M+H]⁺

1H NMR ((400 MHz, CDCl₃) δ 8.13 (s, 3H), 7.39-7.25 (m, 18H), 6.37 (s, 4H), 5.27 (s, 8H), 4.68 (s, 8H), 3.39-3.03 (m, 9H), 2.72 (d, J=44.6, 8H), 2.10 (d, J=63.3, 15H), 1.50-1.34 (m, 40H)), and 13C NMR ((101 MHz, CDCl3) δ 166.8, 144.1, 130.1, 129.2, 129.3, 128.5, 83.2, 74.9, 55.9, 46.9, 38.8, 28.2, 20.2).

### Example A

### 2,2′,2″,2′″-(propane-1,3-diylbis{nitrilobis[(ethane-2,1-diyl)carbamoyl(3-hydroxy-6-methyl-2-oxopyridine-4,1(2H)-diyl)]})tetraacetic acid

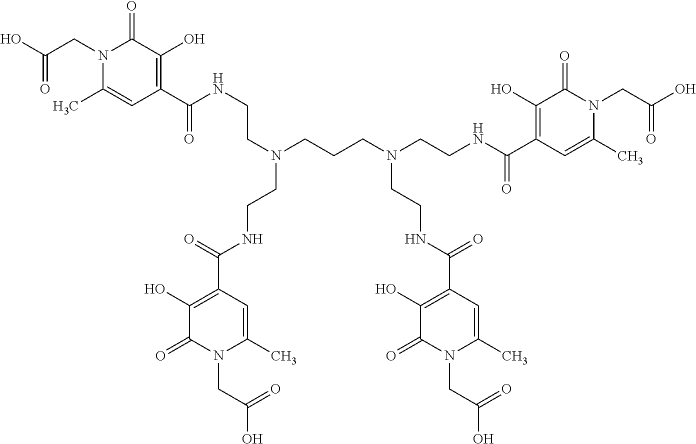 [SMILES: Cc1cc(C(=O)NCCN(CCCN(CCNC(=O)c2cc(C)n(CC(=O)O)c(=O)c2O)CCNC(=O)c2cc(C)n(CC(=O)O)c(=O)c2O)CCNC(=O)c2cc(C)n(CC(=O)O)c(=O)c2O)c(O)c(=O)n1CC(=O)O]

Tetra-tert-butyl 2,2′,2″,2′″-((5,9-bis(2-formamidoethyl)-2,5,9,12-tetraazatridecanedioyl)-tetrakis-(3-(benzyloxy)-6-methyl-2-oxopyridine-4,1(2H)-diyl))tetraacetate (2.70 grams) was treated with concentrated hydrochloric acid (100 mL) at room temperature for 3 hours and concentrated to dryness by evaporation in vacuo. The residue was purified by reverse phase flash chromatography (0-50% ACN in water) to give 2.2 grams of the target compound. This material was again purified using preparative HPLC (column: Phenomenex Luna 5 μm C18(2) 100 Å, 250×50 mm; mobile phase: water/0.1% TFA; ACN; gradient: 0-30% B over 40 min; flow: 50 mL/min; detection: UV 280/335 nm) to afford 400 mg of the target compound.

LC-MS (Method K, gradient: 5-30% B over 3 min): Rₜ=1.68 min; MS (ESIpos): m/z=1083.5 [M+H]⁺

1H NMR ((400 MHz, DMSO) δ 11.03 (4H), 8.62 (s, 4H), 6.39 (s, 4H), 4.74 (s, 8H), 4.16-2.85 (m, 26H), 2.19 (s, 12H)), and 13C NMR ((101 MHz, DMSO) δ 169.3, 165.5, 158.9, 144.3, 134.3, 117.5, 102.9, 51.7, 46.1, 34.6, 19.1)

Alternative Synthesis Route

Intermediate i

### Ethyl 3-hydroxy-6-methyl-2-oxo-1H-pyridine-4-carboxylate

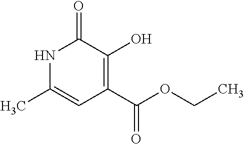

Compound diethyl oxalacetate sodium salt (250 g, 1.19 mol) was partitioned between aq. HCl (1 M, 1.7 L) and DCM (2 L). The layers were stirred at rt for 3 hours until all the solid was dissolved and the pH of the aqueous phase <3. The reaction was parallel performed for 2 batches and worked together. The organic phase (2 batches) was separated and the aqueous phase was extracted with DCM (400 mL×2). The combined organic phase was dried over MgSO₄, filtered and concentrated under reduced pressure to give diethyl 2-hydroxybut-2-enedioate (460 g, crude) as a yellow oil.

To a solution of diethyl 2-hydroxybut-2-enedioate (460 g, the above yellow oil) in THF (4.5 L) was added 1-chloropropan-2-one (285 g, 3.08 mol) at rt. Then NH₃ (g) was bubbled through the reaction mixture for 1.5 hours at −20° C.-0° C. The resulting mixture was stirred at 85° C. in an autoclave (30 Psi) for 4 hours. The reaction mixture was cooled to rt and filtered. The filter cake was washed with MTBE (300 mL). A yellow solid (440 g) was collected by filtration. The crude product (440 g) was combined with other 2 batches crude product (430 g from 500 g of diethyl oxalacetate sodium salt; 450 g from 500 g of diethyl oxalacetate sodium salt). All the crude product was slurried with aq. HCl (1 M, 9 L). The mixture was filtered. The filter cake was washed with H₂O (500 mL) and dried in high vacuum to give ethyl 3-hydroxy-6-methyl-2-oxo-1H-pyridine-4-carboxylate (612 g, 43.5% yield) as a red solid.

¹H NMR: (DMSO-d₆ 400 MHz) 11.95 (brs, 1H), 9.77 (brs, 1H), 6.08 (s, 1H), 4.29 (q, 2H), 2.10 (s, 3H), 1.27 (t, 3H).

Note, containing ˜0.5 eq NH₄⁺

Intermediate i

### Ethyl 1-(2-tert-butoxy-2-oxo-ethyl)-3-hydroxy-6-methyl-2-oxo-pyridine-4-carboxylate

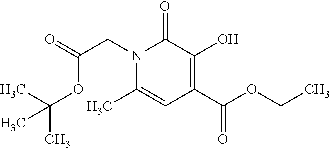 [SMILES: CCOC(=O)c1cc(C)n(CC(=O)OC(C)(C)C)c(=O)c1O]

A mixture of ethyl 3-hydroxy-6-methyl-2-oxo-1H-pyridine-4-carboxylate (50 g, 253.57 mmol), KOtBu (31.50 g, 280.72 mmol) and ditert-butoxymagnesium (95.50 g, 560.01 mmol) in THF (1.2 L) was stirred at rt under N₂ for 1 hour. Tert-butyl 2-bromoacetate (50 mL, 338.37 mmol) in THF (300 mL) was added to the reaction mixture drop-wise at rt. The mixture was stirred at rt for 2 hours. The reaction mixture was concentrated under reduced pressure. The residue was diluted with EA (1 L) and cold sat. aq. citric acid (500 mL). Two phases were separated. The organic phase was washed with H₂O (200 mL×2) and brine (200 mL), dried over Na₂SO₄, filtered and concentrated under reduced pressure. The reaction was parallel performed for 6 batches and all of the residues were combined. The combined crude product was diluted with DCM (2 L) and filtered. The filtrate was concentrated under reduced pressure. The residue was slurried with MTBE (1.5 L) to give ethyl 1-(2-tert-butoxy-2-oxo-ethyl)-3-hydroxy-6-methyl-2-oxo-pyridine-4-carboxylate (314 g, 66.3% yield) as an off-white solid.

¹H NMR: (CDCl₃ 400 MHz) 10.30 (brs, 1H), 6.34 (s, 1H), 4.74 (s, 2H), 4.40 (q, 2H), 2.24 (s, 3H), 1.47 (s, 9H), 1.41 (t, 3H).

Intermediate k

### Ethyl 3-benzyloxy-1-(2-tert-butoxy-2-oxo-ethyl)-6-methyl-2-oxo-pyridine-4-carboxylate

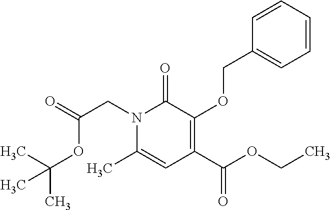 [SMILES: CCOC(=O)c1cc(C)n(CC(=O)OC(C)(C)C)c(=O)c1OCc1ccccc1]

A mixture of ethyl 1-(2-tert-butoxy-2-oxo-ethyl)-3-hydroxy-6-methyl-2-oxo-pyridine-4-carboxylate (183 g, 587.80 mmol), K₂CO₃ (122 g, 882.74 mmol) and BnBr (84 mL, 707.23 mmol) in CH₃CN (1.9 L) was stirred at reflux under N₂ for 3 hours. The reaction mixture was cooled to rt and filtered. The reaction was parallel performed for 2 batches. The combined filtrate was concentrated under reduced pressure to give 500 g of crude product. The crude product was purified with another batch crude product (220 g, 150 g of ethyl 1-(2-tert-butoxy-2-oxo-ethyl)-3-hydroxy-6-methyl-2-oxo-pyridine-4-carboxylate was used in this batch from EW99-2305) together by silica gel chromatography (PE:EA=10:1 to 3:1) to give ethyl 3-benzyloxy-1-(2-tert-butoxy-2-oxo-ethyl)-6-methyl-2-oxo-pyridine-4-carboxylate (615 g, 92.5% yield) as a red oil.

¹H NMR: (CDCl₃ 400 MHz) 7.51-7.49 (m, 2H), 7.35-7.30 (m, 3H), 6.21 (d, 1H), 5.25 (s, 2H), 4.75 (s, 2H), 4.28 (q, 2H), 2.27 (s, 3H), 1.49 (s, 9H), 1.28 (t, 3H).

Intermediate l

### 3-benzyloxy-1-(2-tert-butoxy-2-oxo-ethyl)-6-methyl-2-oxo-pyridine-4-carboxylic acid

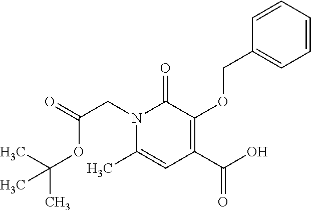 [SMILES: Cc1cc(C(=O)O)c(OCc2ccccc2)c(=O)n1CC(=O)OC(C)(C)C]

To a solution of ethyl 3-benzyloxy-1-(2-tert-butoxy-2-oxo-ethyl)-6-methyl-2-oxo-pyridine-4-carboxylate (102.5 g, 255.32 mmol) in THF (1 L) and H₂O (1 L) was added LiOH.H₂O (12.86 g, 306.39 mmol) in H₂O (300 mL) at rt. The mixture was stirred at rt for 5 hours and then adjusted pH to 3 with sat. aq. citric acid. The resulting mixture was diluted with EA (1.5 L). Two phases were separated. The organic phase was washed with H₂O (500 mL) and brine (500 mL), dried over Na₂SO₄, filtered and concentrated under reduced pressure. The reaction was parallel performed for 6 batches. All of the crude products were slurried with a mixed solvent (1.8 L, PE:EA=10:1) to give 3-benzyloxy-1-(2-tert-butoxy-2-oxo-ethyl)-6-methyl-2-oxo-pyridine-4-carboxylic acid (452 g, 79.0% yield) as a white solid.

¹H NMR: (CDCl₃ 400 MHz) 7.44-7.42 (m, 2H), 7.39-7.37 (m, 3H), 6.58 (d, 1H), 5.57 (s, 2H), 4.75 (s, 2H), 2.29 (s, 3H), 1.51 (s, 9H).

LC-MS: (method L) t=0.789 min, m/z=374.2 (M+H)⁺.

Intermediate m

### N¹-trityl-N²-(2-(tritylamino)ethyl)ethane-1,2-diamine

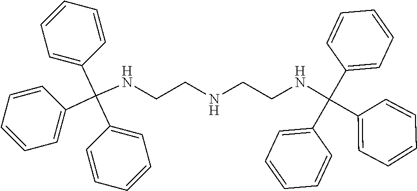 [SMILES: c1ccc(C(NCCNCCNC(c2ccccc2)(c2ccccc2)c2ccccc2)(c2ccccc2)c2ccccc2)cc1]

The reactions were performed as 4 batches in parallel: to a solution of N′-(2-aminoethyl)ethane-1,2-diamine (50 g, 484.66 mmol) in i-PrOH (2 L) was added diethylamine (110 mL, 1.07 mol), followed by TrtCl (270 g, 968.52 mmol) in portions at 0° C. The mixture was stirred at room temperature for 16 hours. TLC (DCM:MeOH=20:1) indicated the reaction completed. NaOH (aq., 5 M, 300 mL for each batch) was added and the mixture was stirred at room temperature for 0.5 h. The suspension of 4 batches were combined and filtered. The cake was washed with water (1 L×6) and dried under air. The crude product was stirred in refluxing MeOH (2.4 L) for 20 min and then filtered while hot. The cake was washed with MeOH (200 mL) and dried in vacuum. The resulting solid was slurried in MeCN (3.0 L) to afford N¹-trityl-N²-(2-(tritylamino)ethyl)ethane-1,2-diamine (741 g, 90% purity) as a white solid.

¹H NMR: (CDCl₃ 400 MHz) 7.52-7.50 (m 12H), 7.28-7.17 (m, 18H), 2.68 (t, 4H), 2.28 (t, 4H).

Intermediate n

### N¹,N¹′-(propane-1,3-diyl)bis(N²-trityl-N¹-(2-(tritylamino)ethyl)ethane-1,2-diamine)

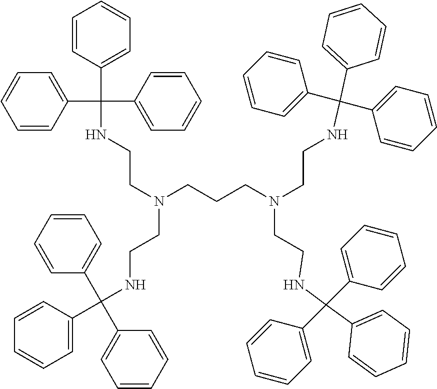 [SMILES: c1ccc(C(NCCN(CCCN(CCNC(c2ccccc2)(c2ccccc2)c2ccccc2)CCNC(c2ccccc2)(c2ccccc2)c2ccccc2)CCNC(c2ccccc2)(c2ccccc2)c2ccccc2)(c2ccccc2)c2ccccc2)cc1]

The reactions were performed as 3 batches in parallel: to a suspension of N¹-trityl-N²-(2-(tritylamino)ethyl)ethane-1,2-diamine (240 g, 367.48 mmol, 90% purity) and 1,3-diiodopropane (60.38 g, 204.06 mmol) in MeCN (1.65 L) was added K₂CO₃ (240 g, 1.74 mol), followed by DMF (3.2 mL, 41.59 mmol). The mixture was stirred at reflux for 3 days under N₂. The mixture was filtered. The cake was washed with water (3.5 L×3) and MeCN (1.5 L) and then stirred in MeCN (3 L) at 70° C. for 0.5 h. The suspension was filtered while hot, and the cake was washed with MeCN (2.5 L). The resulting solid was stirred in MeCN (2 L) at 70° C. for 20 hours and then filtered while hot. The cake was washed with MeCN (2.5 L) and water (4 L) and then dried in vacuum to afford N¹,N¹′-(propane-1,3-diyl)bis(N²-trityl-N¹-(2-(tritylamino)ethyl)ethane-1,2-diamine) (540 g, 444.21 mmol) as a white solid.

¹H NMR: (CDCl₃ 400 MHz) 7.46-7.44 (m 24H), 7.22-7.14 (m, 36H), 2.33 (t, 8H), 2.15 (t, 8H), 1.95 (t, 4H), 1.85 (t, 4H), 1.19 (s, 2H).

Intermediate o

### N¹,N¹′-(propane-1,3-diyl)bis(N¹-(2-aminoethyl)ethane-1,2-diamine)

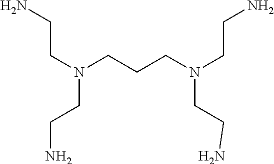 [SMILES: NCCN(CCN)CCCN(CCN)CCN]

The reactions were performed as 3 batches in parallel: a suspension of N¹,N¹-(propane-1,3-diyl)bis(N²-trityl-N¹-(2-(tritylamino)ethyl)ethane-1,2-diamine) (177 g, 145.60 mmol) in HCl (aq., 6 M, 900 mL) was stirred at reflux for 2 hours. MTBE (1 L) was added and the mixture was refluxed for further 1 hour. The organic layer was removed. The aqueous phase was washed with MTBE (1.5 L×5) and lyophilized. The resulting solid was combined with two batches of product to give N¹,N¹-(propane-1,3-diyl)bis(N¹-(2-aminoethyl)ethane-1,2-diamine) hydrochloride (234 g, 503.05 mmol, 6HCl), which was dissolved in H₂O (2 L). NaOH (126 g, 3.15 mol) was added in portions. The mixture was stirred at room temperature for 10 min and then lyophilized. The solid obtained was stirred in DCM (2.5 L) for 10 min and then filtered. The filtrate was concentrated in vacuum. The residue was dissolved in water (300 mL) and then lyophilized to give N¹,N¹-(propane-1,3-diyl)bis(N¹-(2-aminoethyl)ethane-1,2-diamine) (107 g, 86.33% yield) as a yellow oil.

¹H NMR: (D₂O 400 MHz) 2.71-2.67 (m 8H), 2.54-2.50 (m, 8H), 2.43 (t, 4H), 1.62-1.56 (m, 2H).

IC: Average Cl⁻ content: 3.1%.

Intermediate p

### tetra-tert-butyl 2,2′,2″,2′″-[propane-1,3-diylbis(nitrilobis{(ethane-2,1-diyl)carbamoyl[3-(benzyloxy)-6-methyl-2-oxopyridine-4,1(2H)-diyl]})]tetraacetate

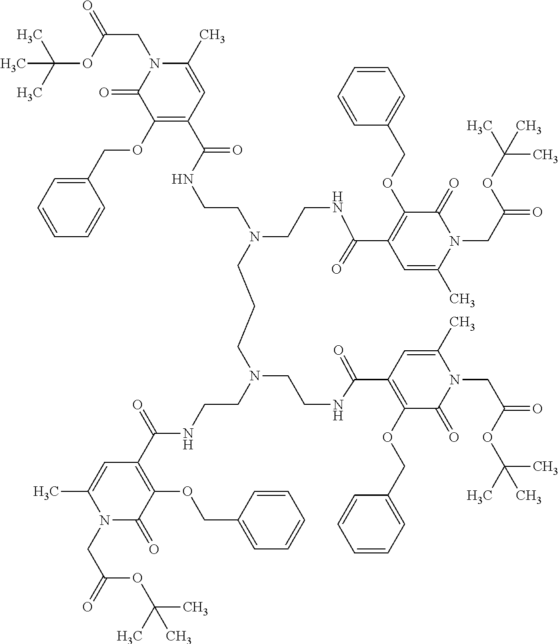 [SMILES: Cc1cc(C(=O)NCCN(CCCN(CCNC(=O)c2cc(C)n(CC(=O)OC(C)(C)C)c(=O)c2OCc2ccccc2)CCNC(=O)c2cc(C)n(CC(=O)OC(C)(C)C)c(=O)c2OCc2ccccc2)CCNC(=O)c2cc(C)n(CC(=O)OC(C)(C)C)c(=O)c2OCc2ccccc2)c(OCc2ccccc2)c(=O)n1CC(=O)OC(C)(C)C]

Under argon 24.2 g (64.9 mmol) of 3-(benzyloxy)-1-(2-tert-butoxy-2-oxoethyl)-6-methyl-2-oxo-1,2-dihydropyridine-4-carboxylic acid were dissolved in 80 mL of N,N-dimethylacetamide. 8.4 g (64.9 mmol) of N,N-diisopropylethylamine and 24.7 g (64.9 mmol) of HATU (CAS-RN:[148893-10-1]) were added at room temperature. The reaction mixture is stirred 30 min at this temperature. Then 4.00 g (16.2 mmol) of N¹,N¹,N³,N³-tetrakis(2-aminoethyl)propane-1,3-diamine dissolved in 20 mL of N,N-dimethylacetamide were added dropwise. The reaction mixture was stirred 2 h at room temperature. Water was added and the formed viscous solid was separated from the liquid phase. The remaining solid residue was dissolved in 400 mL ethyl acetate and extracted with saturated aqueous NaHCO₃-solution. The organic phase is washed twice with water and dried over MgSO₄. The solvent was evaporated under reduced pressure. The residue is purified by using column chromatography A followed by a second column chromatography B (A: Biotage autopurifier system (Isolera LS®), 340 g Biotage SNAP cartridge KP-Sil® ultra column, dichloromethane/ethyl acetate to ethyl acetate/methanol: 0-100% ethyl acetate to 5-10% methanol. B: Biotage autopurifier system (Isolera LS®), 375 g Biotage SNAP cartridge KP-NH®, dichloromethane/ethyl acetate: 0-50% ethyl acetate). Yield: 72%, 19.5 g (11.7 mmol).

¹H NMR (400 MHz, DMSO-d₆) b ppm 1.43 (br s, 38H) 2.21 (s, 12H) 2.31-2.38 (m, 4H) 2.42 (br t, J=6.72 Hz, 8H) 3.13-3.26 (m, 8H) 4.73 (s, 8H) 5.13 (s, 8H) 6.10-6.29 (m, 4H) 7.23-7.33 (m, 12H) 7.35-7.40 (m, 6H) 8.19 (t, J=5.45 Hz, 4H)

LC-MS: R₁=1.68 min; MS (ESIpos): m/z=1668 [M+H]⁺

Instrument: Waters Acquity UPLCMS SingleQuad; column: XBridge C18 5 μm, 150×4.6 mm; eluent A: water+0.2 vol % aqueous ammonia (32%), eluent B: acetonitrile; gradient: 0-1.6 min 1-99% B, 1.6-2.0 min 99% B; flow 0.8 ml/min; temperature: 60° C.; DAD scan: 210-400 nm.

### Example A1

### 2,2′,2″,2′″-(propane-1,3-diylbis{nitrilobis[(ethane-2,1-diyl)carbamoyl(3-hydroxy-6-methyl-2-oxopyridine-4,1(2H)-diyl)]})tetraacetic acid-hydrogen chloride (1/2)

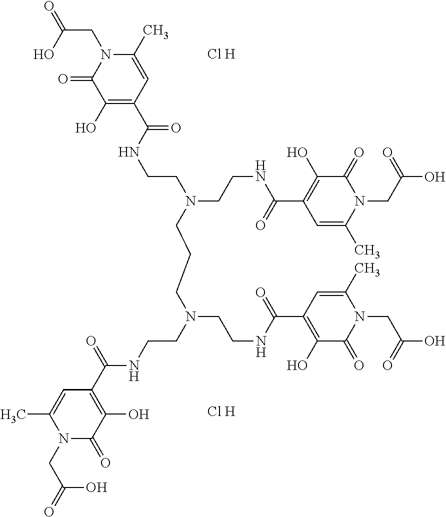 [SMILES: Cc1cc(C(=O)NCCN(CCCN(CCNC(=O)c2cc(C)n(CC(=O)O)c(=O)c2O)CCNC(=O)c2cc(C)n(CC(=O)O)c(=O)c2O)CCNC(=O)c2cc(C)n(CC(=O)O)c(=O)c2O)c(O)c(=O)n1CC(=O)O ; "Cl H" ; "Cl H"]

23.2 g (13.9 mmol) of tetra-tert-butyl 2,2′,2″,2′-[propane-1,3-diylbis(nitrilobis{(ethane-2,1-diyl)carbamoyl[3-(benzyloxy)-6-methyl-2-oxopyridine-4,1(2H)-diyl]})]tetraacetate were suspended in 115 mL aqueous hydrochloric acid (37%). The mixture was stirred 16 h at room temperature and then 2 h at 50° C. Afterwards the mixture was stirred further 48 h at room temperature. 500 mL of 2-methyltetrahydrofuran were added at room temperature and the yellow precipitate was collected by filtration. After washing with a small amount of ethanol the residue was titurated in 50 mL of ethanol at 45° C. Yield: 96%, 15.5 g (13.4 mmol).

¹H NMR (400 MHz, DMSO-d₆) b ppm 2.03-2.33 (m, 14H) 3.66-3.86 (br m, 12H) 4.74 (s, 8H) 6.46 (s, 4H) 8.64-8.82 (br m, 4H) 10.49-10.60 (br s, 2H) 11.09-11.22 (br s, 2H) 12.89-13.51 (br s, 4H)

LC-MS: R₁=0.50 min; MS (ESIpos): m/z=1083 [M+H]⁺

Instrument: Waters Acquity UPLCMS SingleQuad; Column: XBridge C18 5 μm, 150×4.6 mm; Eluent A: water+0.1 Vol-% formic acid (99%), Eluent B: acetonitrile; Gradient: 0-1.7 min 1-45% B, 1.7-1.72 min 45-99% B, 1.72-2.0 min 99% B; Flow 0.8 ml/min; Temperature: 60° C.; ELSD.

Small Molecule Conjugates Targeting PSMA

Intermediate 1

### di-tert-butyl N-{[(2S)-6-{[(benzyloxy)carbonyl]amino}-1-tert-butoxy-1-oxohexan-2-yl]carbamoyl}-L-glutamate

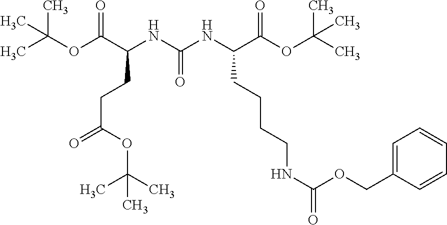 [SMILES: CC(C)(C)OC(=O)CC[C@H](NC(=O)N[C@@H](CCCCNC(=O)OCc1ccccc1)C(=O)OC(C)(C)C)C(=O)OC(C)(C)C]

tert-butyl N6-[(benzyloxy)carbonyl]-L-lysinate-hydrogen chloride (1/1) (7.27 g, 19.5 mmol; CAS-RN:[5978-22-3]) was solubilised in DCM (100 ml), cooled to 0° C. under argon, and N,N-diisopropylethylamine (14 ml, 78 mmol) was added dropwise. The mixture was stirred for 5 min at 0° C. and 30 min at rt. 4-nitrophenyl carbonochloridate (3.59 g, 17.8 mmol; CAS-RN:[7693-46-1]) and di-tert-butyl L-glutamate-hydrogen chloride (1/1) (5.00 g, 16.9 mmol; CAS-RN:[32677-01-3]) were added and N,N-diisopropylethylamine (14 ml, 78 mmol) was added dropwise to the mixture. It was stirred overnight at rt. The mixture was concentrated under reduced pressure, diluted with DCM, washed 2 times with sodium hydroxide (0.5 M) and once with brine. The organic layer was dried and evaporated. The residue was purified by flash chromatography (SiO2, hexane/EtOAc gradient 0%-50%) to give 8.40 g (96% purity, 67% yield) of the target compound.

LC-MS (Method 1): Rt=1.48 min; MS (ESIpos): m/z=623 [M+H]⁺

¹H-NMR (400 MHz, DMSO-d6) δ[ppm]: 1.154 (0.66), 1.172 (1.40), 1.190 (0.71), 1.384 (16.00), 1.392 (7.49), 1.987 (2.48), 3.331 (2.46), 4.017 (0.64), 4.035 (0.68), 4.992 (1.08), 7.318 (0.43), 7.334 (1.05), 7.342 (0.98), 7.360 (0.43).

Intermediate 2

### di-tert-butyl N-{[(2S)-6-amino-1-tert-butoxy-1-oxohexan-2-yl]carbamoyl}-L-glutamate

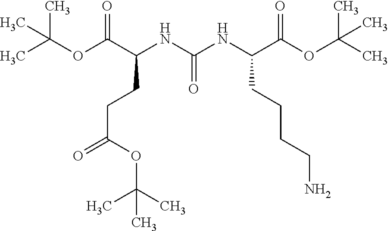 [SMILES: CC(C)(C)OC(=O)CC[C@H](NC(=O)N[C@@H](CCCCN)C(=O)OC(C)(C)C)C(=O)OC(C)(C)C]

di-tert-butyl N-{[(2S)-6-{[(benzyloxy)carbonyl]amino}-1-tert-butoxy-1-oxohexan-2-yl]carbamoyl}-L-glutamate (8.48 g, 13.6 mmol) was solubilised in MeOH (42 ml), palladium on carbon (1.45 g, 10% purity, 1.36 mmol) was added and the mixture was purged with hydrogen. The mixture was stirred for 4 h at rt under hydrogen atmosphere. The mixture was filtered over Celite, washed with MeOH and evaporated. The residue was diluted with DCM, washed 3 times with sat. sodium hydrogen carbonate solution and once with brine. The organic layer was dried and evaporated. The residue was purified by flash chromatography (SiO2, DCM/Ethanol gradient 0%-20%) to give 4.06 g (97% purity, 59% yield) of the target compound.

LC-MS (Method 1): Rₜ=0.97 min; MS (ESIpos): m/z=489 [M+H]⁺

¹H-NMR (400 MHz, DMSO-d6) δ[ppm]: 1.053 (0.43), 1.288 (0.42), 1.297 (0.48), 1.302 (0.48), 1.386 (16.00), 1.390 (13.68), 1.396 (13.08), 2.477 (0.52), 3.165 (1.50), 3.330 (0.58), 6.239 (0.40).

Intermediate 3

### tri-tert-butyl (5S,12S,16S)-1-(9H-fluoren-9-yl)-5-[(naphthalen-2-yl)methyl]-3,6,14-trioxo-2-oxa-4,7,13,15-tetraazaoctadecane-12,16,18-tricarboxylate

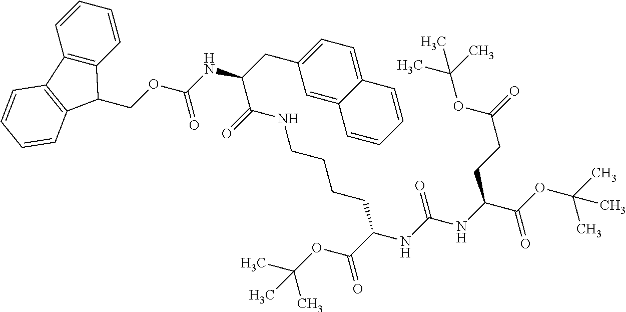 [SMILES: CC(C)(C)OC(=O)CC[C@H](NC(=O)N[C@@H](CCCCNC(=O)[C@H](Cc1ccc2ccccc2c1)NC(=O)OCC1c2ccccc2-c2ccccc21)C(=O)OC(C)(C)C)C(=O)OC(C)(C)C]

di-tert-butyl N-{[(2S)-6-amino-1-tert-butoxy-1-oxohexan-2-yl]carbamoyl}-L-glutamate (1.00 g, 70% purity, 1.44 mmol) and (2S)-2-({[(9H-fluoren-9-yl)methoxy]carbonyl}amino)-3-(naphthalen-2-yl)propanoic acid (940 mg, 2.15 mmol; CAS-RN:[112883-43-9]) were solubilised in DMF (11 ml), 4-methylmorpholine (470 μl, 4.3 mmol, CAS-RN: 109-02-4) and HATU (819 mg, 2.15 mmol) were added and the mixture was stirred under argon overnight at rt. The mixture was evaporated and purified by preparative HPLC (C18, acetonitrile/water with 0.1% formic acid) to give 1.11 g (93% purity, 79% yield) of the target compound.

LC-MS (Method 1): Rₜ=1.67 min; MS (ESIpos): m/z=908 [M+H]⁺

¹H-NMR (400 MHz, DMSO-d6) δ[ppm]: 1.379 (16.00), 2.297 (2.06), 4.101 (0.61), 4.107 (0.70), 7.164 (0.59), 7.181 (0.62), 7.230 (0.56), 7.249 (0.74), 7.363 (0.42), 7.457 (0.50), 7.758 (0.43), 7.844 (0.68), 7.863 (0.60).

Intermediate 4

### di-tert-butyl (2S)-2-({[(2S)-6-{[(2S)-2-amino-3-(naphthalen-2-yl)propanoyl]amino}-1-tert-butoxy-1-oxohexan-2-yl]carbamoyl}amino)pentanedioate

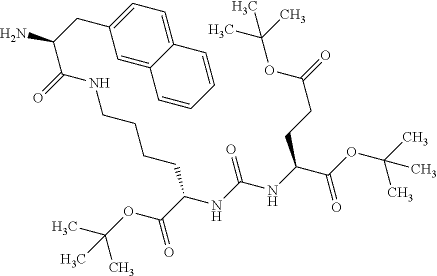 [SMILES: CC(C)(C)OC(=O)CC[C@H](NC(=O)N[C@@H](CCCCNC(=O)[C@@H](N)Cc1ccc2ccccc2c1)C(=O)OC(C)(C)C)C(=O)OC(C)(C)C]

tri-tert-butyl (5S,12S,16S)-1-(9H-fluoren-9-yl)-5-[(naphthalen-1-yl)methyl]-3,6,14-trioxo-2-oxa-4,7,13,15-tetraazaoctadecane-12,16,18-tricarboxylate (1.11 g, 93% purity, 1.14 mmol) was solubilised in DMF (26 ml), piperidine (2.3 ml, 23 mmol) was added and the mixture was stirred under argon overnight at rt. The mixture was evaporated and purified by preparative HPLC (C18, acetonitrile/water with 0.1% formic acid) to give 663 mg (85% yield) of the target compound.

LC-MS (Method 1): Rₜ=1.22 min; MS (ESIpos): m/z=686 [M+H]⁺

¹H-NMR (400 MHz, DMSO-d6) δ[ppm]: 1.382 (13.90), 1.387 (16.00), 2.297 (3.11), 7.162 (0.66), 7.180 (0.78), 7.230 (0.67), 7.249 (0.71), 7.455 (0.48), 7.679 (0.55), 7.806 (0.81), 7.828 (0.77), 7.845 (0.55).

Intermediate 5

### tri-tert-butyl (3S,10S,14S)-1-{(1r,4S)-4-[({[(9H-fluoren-9-yl)methoxy]carbonyl}amino)methyl]cyclohexyl}-3-[(naphthalen-2-yl)methyl]-1,4,12-trioxo-2,5,11,13-tetraazahexadecane-10,14,16-tricarboxylate

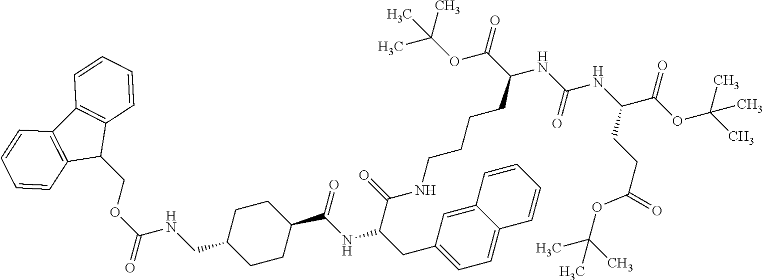

di-tert-butyl (2S)-2-({[(2S)-6-{[(2S)-2-amino-3-(naphthalen-2-yl)propanoyl]amino}-1-tert-butoxy-1-oxohexan-2-yl]carbamoyl}amino)pentanedioate (116 mg, 70% purity, 119 μmol) and (1R,4R)-4-[({[(9H-fluoren-9-yl)methoxy]carbonyl}amino)methyl]cyclohexane-1-carboxylic acid (67.5 mg, 178 μmol) were solubilised in DMF (910 μl), 4-methylmorpholine (39 μl, 360 μmol, CAS-RN: 109-02-4) and HATU (67.6 mg, 178 μmol) were added and the mixture was stirred under argon overnight at rt. The mixture was evaporated and purified by preparative HPLC (C18, acetonitrile/water with 0.1% formic acid) to give 134 mg (80% purity, 86% yield) of the target compound.

LC-MS (Method 1): Rₜ=1.66 min; MS (ESIpos): m/z=1047 [M+H]⁺

¹H-NMR (400 MHz, DMSO-d6) δ[ppm]: 1.231 (0.44), 1.378 (13.79), 1.385 (16.00), 2.298 (0.55), 2.518 (1.25), 2.522 (0.78), 4.279 (0.64), 4.296 (0.45), 7.306 (0.61), 7.324 (0.42), 7.381 (0.59), 7.400 (0.75), 7.418 (0.40), 7.448 (0.45), 7.668 (0.63), 7.685 (0.77), 7.789 (0.50), 7.874 (0.67), 7.893 (0.61), 7.947 (0.40).

Intermediate 6

### tri-tert-butyl (3S,10S,14S)-1-[(1r,4S)-4-(aminomethyl)cyclohexyl]-3-[(naphthalen-2-yl)methyl]-1,4,12-trioxo-2,5,11,13-tetraazahexadecane-10,14,16-tricarboxylate

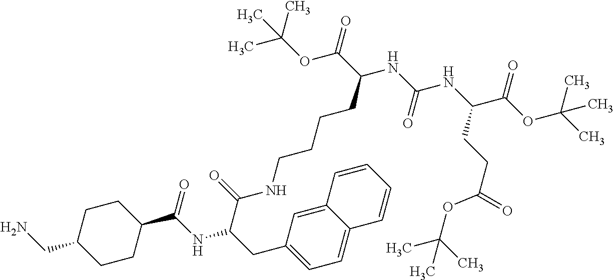

tri-tert-butyl (3S,10S,14S)-1-{(1R,4S)-4-[({[(9H-fluoren-9-yl)methoxy]carbonyl}amino)methyl]cyclohexyl}-3-[(naphthalen-2-yl)methyl]-1,4,12-trioxo-2,5,11,13-tetraazahexadecane-10,14,16-tricarboxylate (730 mg, 98% purity, 684 μmol) was solubilised in DMF (18 ml), piperidine (1.4 ml, 14 mmol) was added and the mixture was stirred under argon overnight at rt. The mixture was evaporated and purified by preparative HPLC (C18, acetonitrile/water with 0.1% formic acid) to give 381 mg (98% purity, 66% yield) of the target compound.

LC-MS (Method 1): Rₜ=1.20 min; MS (ESIpos): m/z=825 [M+H]⁺

¹H-NMR (400 MHz, DMSO-d6) δ[ppm]: 1.381 (14.66), 1.387 (16.00), 2.518 (1.24), 2.523 (0.84), 3.342 (0.66), 7.451 (0.53), 7.678 (0.48), 7.785 (0.58), 8.419 (0.62).

### Example 1-A

### (3S,10S,14S)-1-[(1r,4S)-4-(aminomethyl)cyclohexyl]-3-[(naphthalen-2-yl)methyl]-1,4,12-trioxo-2,5,11,13-tetraazahexadecane-10,14,16-tricarboxylic acid

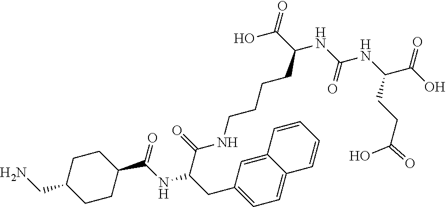

tri-tert-butyl (3S,10S,14S)-1-[(1r,4S)-4-(aminomethyl)cyclohexyl]-3-[(naphthalen-2-yl)methyl]-1,4,12-trioxo-2,5,11,13-tetraazahexadecane-10,14,16-tricarboxylate (25.0 mg, 98% purity, 29.7 μmol) was solubilised in DCM (1.0 ml), TFA (1.0 ml, 13 mmol) was added and the mixture was stirred under argon for 2 h at rt. The mixture was evaporated and purified by preparative HPLC (C18, acetonitrile/water with 0.1% formic acid) to give 12.0 mg (95% purity, 58% yield) of the target compound.

LC-MS (Method 1): Rₜ=0.70 min; MS (ESIpos): m/z=657 [M+H]⁺

¹H-NMR (400 MHz, DMSO-d6) δ[ppm]: 1.232 (1.18), 1.904 (1.25), 2.327 (3.80), 2.331 (2.75), 2.336 (1.25), 2.518 (16.00), 2.523 (10.10), 2.558 (0.52), 2.577 (0.52), 2.669 (3.87), 2.673 (2.75), 2.678 (1.25), 3.119 (0.46), 7.452 (0.59), 7.693 (0.59), 7.783 (0.72), 7.804 (0.59).

The amino group of Intermediate 6 can then used in amide bond forming reactions with the carboxylate containing chelators of this invention to form chelator conjugates containing monomeric, dimeric, trimeric or tetrameric pharmacophore moieties. The protecting groups are removed using standard conditions. The conjugates can also be prepared by activating the chelator moiety followed by addition of the amine-containing pharmacophore (Example 1A).

Intermediate 7

### tri-tert-butyl (3S,10S,14S)-3-[(naphthalen-2-yl)methyl]-1,4,12-trioxo-1-[(1r,4S)-4-({2-[4,7,10-tris(2-tert-butoxy-2-oxoethyl)-1,4,7,10-tetraazacyclododecan-1-yl]acetamido}methyl)cyclohexyl]-2,5,11,13-tetraazahexadecane-10,14,16-tricarboxylate

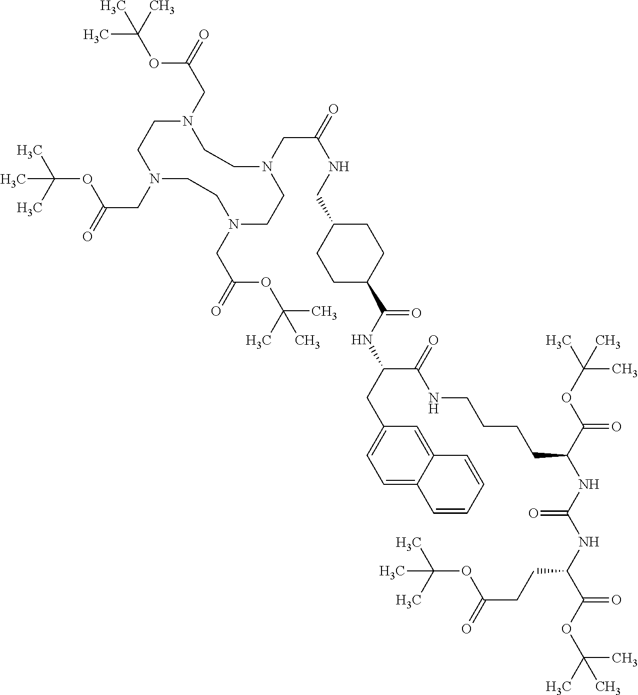 [SMILES: CC(C)(C)OC(=O)CC[C@H](NC(=O)N[C@@H](CCCCNC(=O)[C@H](Cc1ccc2ccccc2c1)NC(=O)[C@H]1CC[C@H](CNC(=O)CN2CCN(CC(=O)OC(C)(C)C)CCN(CC(=O)OC(C)(C)C)CCN(CC(=O)OC(C)(C)C)CC2)CC1)C(=O)OC(C)(C)C)C(=O)OC(C)(C)C]

[4,7,10-tris(2-tert-butoxy-2-oxoethyl)-1,4,7,10-tetraazacyclododecan-1-yl]acetic acid (289 mg, 505 μmol), [(1H-benzotriazol-1-yl)oxy](dimethylamino)-N,N-dimethylmethaniminium hexafluoridophosphate(1−) (189 mg, 498 μmol; CAS-RN:94790-37-1) and N,N-diisopropylethylamine (88 μl, 500 μmol) were stirred in DMF (3.5 ml) for 15 min at rt. tri-tert-butyl (3S,10S,14S)-1-[(1r,4S)-4-(aminomethyl)cyclohexyl]-3-[(naphthalen-2-yl)methyl]-1,4,12-trioxo-2,5,11,13-tetraazahexadecane-10,14,16-tricarboxylate (104 mg, 126 μmol) was added and the mixture was stirred at rt. The mixture was evaporated and purified by preparative HPLC (C18, acetonitrile/water with 0.1% formic acid) to give 97.0 mg (99% purity, 55% yield) of the target compound.

LC-MS (Method 1): Rₜ=1.42 min; MS (ESIpos): m/z=1379 [M+H]⁺

¹H-NMR (400 MHz, DMSO-d6) δ[ppm]: 1.381 (15.02), 1.387 (16.00), 1.407 (4.24), 1.412 (2.61), 1.479 (2.90), 2.332 (0.43), 2.518 (2.40), 2.523 (1.70), 2.673 (0.44), 2.917 (0.45), 3.513 (0.42), 7.449 (0.44).

### Example 1-B, PSMA-617

### (3S,10S,14S)-3-[(naphthalen-2-yl)methyl]-1,4,12-trioxo-1-[(1r,4S)-4-({2-[4,7,10-tris(carboxymethyl)-1,4,7,10-tetraazacyclododecan-1-yl]acetamido}methyl)cyclohexyl]-2,5,11,13-tetraazahexadecane-10,14,16-tricarboxylic acid

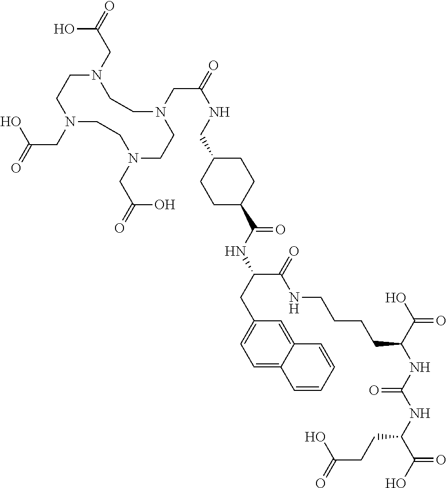 [SMILES: O=C(O)CC[C@H](NC(=O)N[C@@H](CCCCNC(=O)[C@H](Cc1ccc2ccccc2c1)NC(=O)[C@H]1CC[C@H](CNC(=O)CN2CCN(CC(=O)O)CCN(CC(=O)O)CCN(CC(=O)O)CC2)CC1)C(=O)O)C(=O)O]

tri-tert-butyl (3S,10S,14S)-3-[(naphthalen-2-yl)methyl]-1,4,12-trioxo-1-[(1r,4S)-4-({2-[4,7,10-tris(2-tert-butoxy-2-oxoethyl)-1,4,7,10-tetraazacyclododecan-1-yl]acetamido}methyl)cyclohexyl]-2,5,11,13-tetraazahexadecane-10,14,16-tricarboxylate (96.0 mg, 98% purity, 68.2 μmol) was solubilised in DCM (9.6 ml), TFA (79 μl, 1.0 mmol) was added and the mixture was stirred under argon at rt. The mixture was evaporated and purified by preparative HPLC (C18, acetonitrile/water with 0.1% formic acid) to give 48.0 mg (95% purity, 64% yield) of the target compound.

LC-MS (Method 1): Rₜ=0.72 min; MS (ESIneg): m/z=1041 [M−H]⁻

¹H-NMR (400 MHz, DMSO-d6) δ[ppm]: 0.793 (0.86), 0.822 (1.00), 1.093 (0.43), 1.124 (0.43), 1.245 (1.64), 1.313 (1.36), 1.328 (1.64), 1.463 (1.14), 1.572 (1.14), 1.613 (1.14), 1.656 (1.07), 1.699 (0.79), 1.717 (0.64), 1.734 (0.71), 1.751 (0.79), 1.860 (0.57), 1.876 (0.57), 1.907 (1.71), 2.049 (0.79), 2.075 (1.50), 2.214 (1.29), 2.236 (2.00), 2.252 (1.00), 2.337 (1.43), 2.518 (16.00), 2.523 (11.00), 2.540 (10.79), 2.632 (1.93), 2.674 (3.29), 2.679 (1.64), 2.909 (2.14), 2.966 (5.29), 3.074 (2.86), 3.099 (2.00), 3.121 (1.57), 3.134 (1.50), 3.427 (8.07), 3.983 (0.86), 3.994 (0.93), 4.066 (0.86), 4.082 (0.86), 4.463 (0.50), 4.484 (0.79), 4.498 (0.79), 4.521 (0.43), 6.304 (1.71), 6.325 (1.57), 7.389 (1.50), 7.411 (1.64), 7.432 (1.57), 7.435 (1.43), 7.452 (2.43), 7.466 (1.36), 7.469 (1.36), 7.483 (0.57), 7.693 (2.71), 7.785 (3.71), 7.806 (3.29), 7.834 (1.50), 7.839 (1.50), 7.857 (1.43), 7.923 (1.00), 8.068 (1.14), 8.083 (1.21), 8.106 (0.71), 8.142 (1.00).

### Example 1-B-²³²Th

### {3-[(naphthalen-2-yl)methyl]-1,4,12-trioxo-1-{4-[(2-{4,7,10-tris[(carboxy-kappaO)methyl]-1,4,7,10-tetraazacyclododecan-1-yl-kappa⁴N¹,N⁴,N⁷,N¹⁰}acetamido)methyl]cyclohexyl}-2,5,11,13-tetraazahexadecane-10,14,16-tricarboxylato(4−)}thorium(3+)

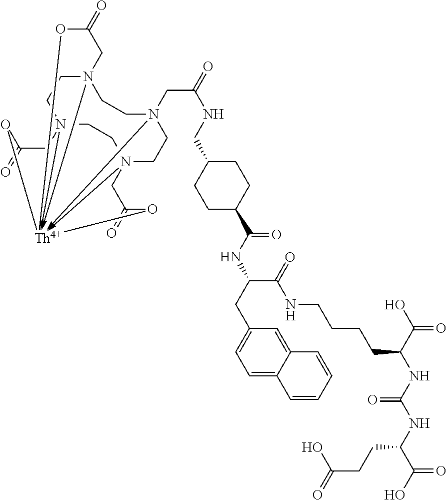

(3S,10S,14S)-3-[(naphthalen-2-yl)methyl]-1,4,12-trioxo-1-[(1r,4S)-4-({2-[4,7,10-tris(carboxymethyl)-1,4,7,10-tetraazacyclododecan-1-yl]acetamido}methyl)cyclohexyl]-2,5,11,13-tetraazahexadecane-10,14,16-tricarboxylic acid (28.6 mg, 27.4 μmol) was solubilised in ammonium acetate (29 ml, 1.0 M, 29 mmol, prepared with ultrafiltered and autoclaved water), thorium solution (6.6 ml, 27 μmol, 1 μg/μl in HNO₃ [2%]) was added and the mixture was stirred for 3.5 h at 90° C. The mixture was evaporated and diluted with DMF. The resulting suspension was filtered, washed with DMF and the filtrate was purified by preparative HPLC (C18, acetonitrile/water with 0.1% formic acid) to give 1.20 mg (75% purity, 3% yield) of the target compound.

¹H-NMR (400 MHz, DMSO-d6) δ[ppm]: 0.851 (0.44), 1.232 (1.27), 1.352 (0.89), 1.452 (0.57), 1.656 (0.57), 2.235 (0.44), 2.322 (2.98), 2.327 (3.87), 2.331 (2.92), 2.336 (1.52), 2.518 (16.00), 2.522 (9.40), 2.539 (1.08), 2.549 (2.29), 2.664 (2.98), 2.669 (3.94), 2.673 (2.98), 2.678 (1.59), 2.727 (0.83), 2.888 (0.83), 2.938 (0.51), 3.088 (0.57), 3.131 (0.51), 3.505 (1.02), 7.085 (0.76), 7.384 (0.51), 7.404 (0.51), 7.428 (0.38), 7.443 (0.89), 7.461 (1.40), 7.476 (0.89), 7.493 (0.44), 7.698 (0.76), 7.801 (1.21), 7.821 (1.02), 7.842 (0.95), 7.863 (0.89).

### Example 1-B-²²⁷Th

### [²²⁷Th]Thorium (3S,10S,14S)-3-[(naphthalen-2-yl)methyl]-1,4,12-trioxo-1-{(1r,4S)-4-[(2-{4,7,10-tris[(carboxy-kappaO)methyl]-1,4,7,10-tetraazacyclododecan-1-yl-kappa⁴N¹,N⁴,N⁷,N¹⁰}acetamido)methyl]cyclohexyl}-2,5,11,13-tetraazahexadecane-10,14,16-tricarboxylate

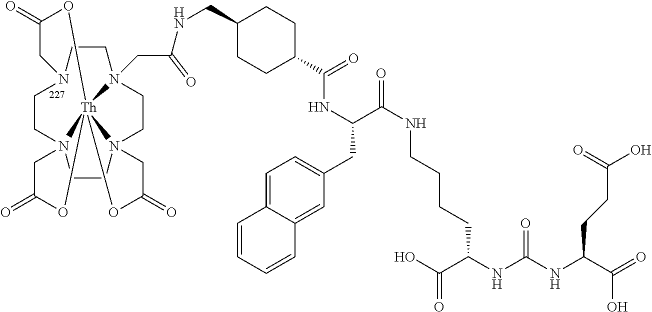

(3S,10S,14S)-3-[(naphthalen-2-yl)methyl]-1,4,12-trioxo-1-[(1r,4S)-4-({2-[4,7,10-tris(carboxymethyl)-1,4,7,10-tetraazacyclododecan-1-yl]acetamido}methyl)cyclohexyl]-2,5,11,13-tetraazahexadecane-10,14,16-tricarboxylic acid dissolved in 400 mM sodium acetate buffer (pH 5.6) containing 0.5 mg/mL pABA was mixed with thorium-227 in 0.5 M HCl at 0.3 MBq/nmol specific activity and RAC of 7.2 MBq/mL at 95° C. for 40 min. The labelling efficiency was determined to be 92% by iTLC.

Intermediate 8

### {4-[(2-{(3-{bis[2-({1-[2-({[(1S,4r)-4-{[(7S,11S,18S)-7,11-bis(tert-butoxycarbonyl)-2,2-dimethyl-19-(naphthalen-2-yl)-4,9,17-trioxo-3-oxa-8,10,16-triazanonadecan-18-yl]carbamoyl}cyclohexyl]methyl}amino)-2-oxoethyl]-3-hydroxy-6-methyl-2-oxo-1,2-dihydropyridine-4-carbonyl}amino)ethyl]amino}propyl)[2-({1-[2-({[(1S,4r)-4-{[(7S,11S,18S)-7,11-bis(tert-butoxycarbonyl)-2,2-dimethyl-19-(naphthalen-2-yl)-4,9,17-trioxo-3-oxa-8,10,16-triazanonadecan-18-yl]carbamoyl}cyclohexyl]methyl}amino)-2-oxoethyl]-3-hydroxy-6-methyl-2-oxo-1,2-dihydropyridine-4-carbonyl}amino)ethyl]amino}ethyl)carbamoyl]-3-hydroxy-6-methyl-2-oxopyridin-1(2H)-yl}acetic acid

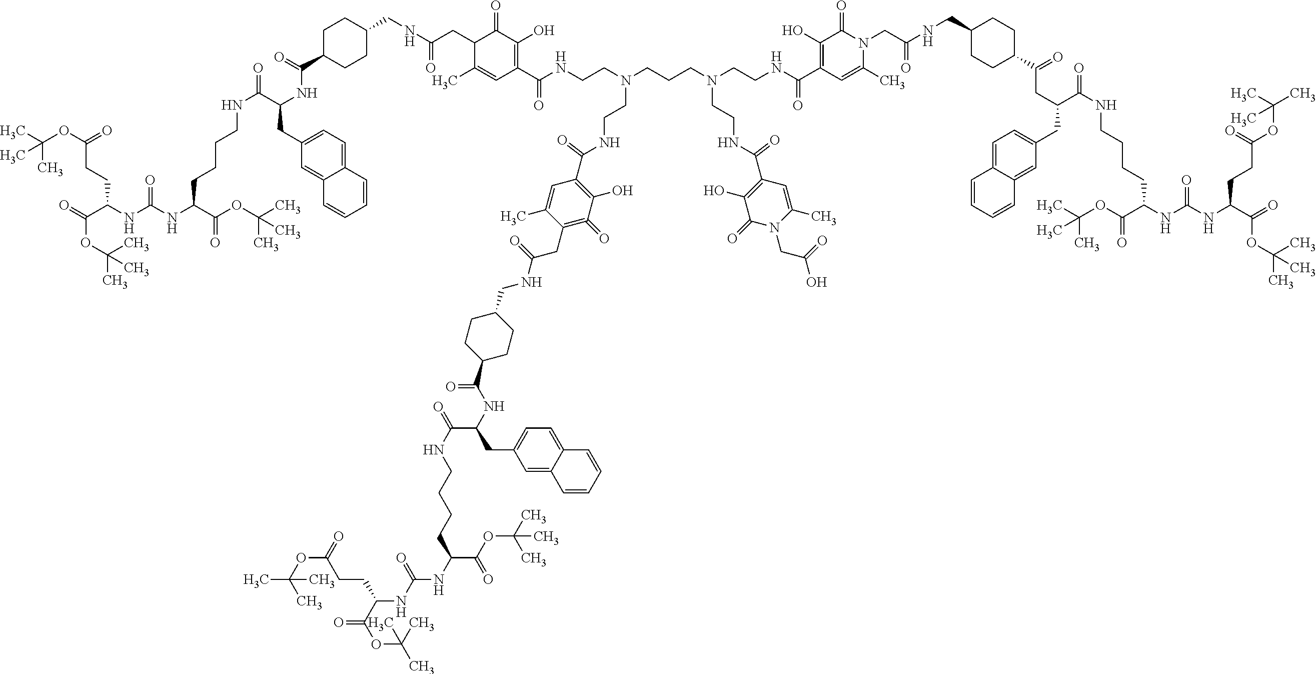

2,2′,2″,2′″-(propane-1,3-diylbis{nitrilobis[(ethane-2,1-diyl)carbamoyl(3-hydroxy-6-methyl-2-oxopyridine-4,1(2H)-diyl)]})tetraacetic acid (5 mg, 4.6 μmol) and tri-tert-butyl (3S,10S,14S)-1-[(1r,4S)-4-(aminomethyl)cyclohexyl]-3-[(naphthalen-2-yl)methyl]-1,4,12-trioxo-2,5,11,13-tetraazahexadecane-10,14,16-tricarboxylate (15.2 mg, 18.5 μmol) are dissolved in NMP (1.0 mL), DIPEA (8.0 μL) is added, then PyAOP (9.6 mg, 18.5 μmol) in NMP (960 μL) is added. Reaction mix is diluted with 50% ACN/water/0.1% TFA (6 mL) and the products purified by preparative HPLC (Äkta pure system, column: Phenomenex Luna 5 μm C18(2) 100 Å, 250×21.2 mm Mobile phase: Water/0.1% TFA; ACN Gradient: 50-100% B over 40 min Flow: 10 mL/min Detection: UV 280/335 nm, tR: 33 min) affording 2.5 mg of the target compound.

LC-MS (Method K, gradient: 50-100% B over 3 min): Rₜ=2.02 min; MS (ESIpos): m/z=1750.5 [M+2H]²⁺

### Example 1-C Monomer

### (3S,10S,14S)-1-[(1r,4S)-4-({2-[4-({2-[{3-[bis(2-{[1-(carboxymethyl)-3-hydroxy-6-methyl-2-oxo-1,2-dihydropyridine-4-carbonyl]amino}ethyl)amino]propyl}(2-{[1-(carboxymethyl)-3-hydroxy-6-methyl-2-oxo-1,2-dihydropyridine-4-carbonyl]amino}ethyl)amino]ethyl}carbamoyl)-3-hydroxy-6-methyl-2-oxopyridin-1(2H)-yl]acetamido}methyl)cyclohexyl]-3-[(naphthalen-2-yl)methyl]-1,4,12-trioxo-2,5,11,13-tetraazahexadecane-10,14,16-tricarboxylic acid

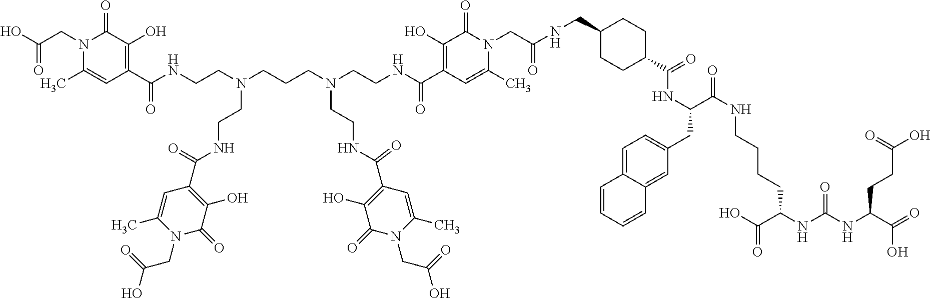 [SMILES: Cc1cc(C(=O)NCCN(CCCN(CCNC(=O)c2cc(C)n(CC(=O)O)c(=O)c2O)CCNC(=O)c2cc(C)n(CC(=O)NC[C@H]3CC[C@H](C(=O)N[C@@H](Cc4ccc5ccccc5c4)C(=O)NCCCC[C@H](NC(=O)N[C@@H](CCC(=O)O)C(=O)O)C(=O)O)CC3)c(=O)c2O)CCNC(=O)c2cc(C)n(CC(=O)O)c(=O)c2O)c(O)c(=O)n1CC(=O)O]

2,2′,2″,2′″-(propane-1,3-diylbis{nitrilobis[(ethane-2,1-diyl)carbamoyl(3-hydroxy-6-methyl-2-oxopyridine-4,1(2H)-diyl)]})tetraacetic acid (5.70 mg, 5.26 μmol) in 570 μL NMP and PyAOP (2.7 mg, 5.26 μmol) in 270 μL NMP are mixed in a vial. DIPEA (4.6 μL, 26 μmol) is added. (3S,10S,14S)-1-[(1r,4S)-4-(aminomethyl)cyclohexyl]-3-[(naphthalen-2-yl)methyl]-1,4,12-trioxo-2,5,11,13-tetraazahexadecane-10,14,16-tricarboxylic acid (5.18 mg, 7.89 μmol) in 520 μL NMP is added. Reaction mix is diluted with 10% ACN/water/0.1% TFA (7 mL) and products purified by preparative HPLC (RP-HPLC using Äkta pure system (ALG-106) Column: Phenomenex Luna 5 μm C18(2) 100 Å, 250×21.2 mm Mobile phase: Water/0.1% TFA; ACN Gradient: 10-50% B over 40 min Flow: 10 mL/min Detection: UV 280/335 nm tR product: 31 min tR by-product: 37 min) affording 4.6 mg of the target compound.

LC-MS (Method K, gradient: 10-50% B over 3 min): Rₜ=1.75 min; MS (ESIpos): m/z=1720.4 [M+H]⁺

### Example 1-C Monomer-²³²Th

### (3S,10S,14S)-1-[(1r,4S)-4-({2-[4-({2-[{3-[bis(2-{[1-(carboxymethyl)-3-(hydroxy-kappaO)-6-methyl-2-(oxo-kappaO)-1,2-dihydropyridine-4-carbonyl]amino}ethyl)amino]propyl}(2-{[1-(carboxymethyl)-3-(hydroxy-kappaO)-6-methyl-2-(oxo-kappaO)-1,2-dihydropyridine-4-carbonyl]amino}ethyl)amino]ethyl}carbamoyl)-3-(hydroxy-kappaO)-6-methyl-2-(oxo-kappaO)pyridin-1(2H)-yl]acetamido}methyl)cyclohexyl]-3-[(naphthalen-2-yl)methyl]-1,4,12-trioxo-2,5,11,13-tetraazahexadecane-10,14,16-tricarboxylato(4−)(²³²Th)Thorium

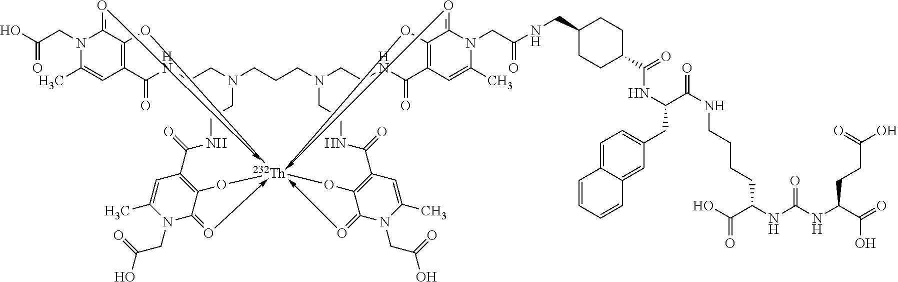

(3S,10S,14S)-1-[(1r,4S)-4-({2-[4-({2-[{3-[bis(2-{[1-(carboxymethyl)-3-hydroxy-6-methyl-2-oxo-1,2-dihydropyridine-4-carbonyl]amino}ethyl)amino]propyl}(2-{[1-(carboxymethyl)-3-hydroxy-6-methyl-2-oxo-1,2-dihydropyridine-4-carbonyl]amino}ethyl)amino]ethyl}carbamoyl)-3-hydroxy-6-methyl-2-oxopyridin-1(2H)-yl]acetamido}methyl)cyclohexyl]-3-[(naphthalen-2-yl)methyl]-1,4,12-trioxo-2,5,11,13-tetraazahexadecane-10,14,16-tricarboxylic acid (1.00 mg, 0.581 μmol) is dissolved in 0.5 M carbonate buffer (0.5 mL), Th-232 solution (1 mg/mL in 2% HNO3, 200 μg, 0.87 μmol) is added. Reaction mixture is lyophilised affording 1.10 mg (95% purity, 92% yield) of the target compound.

LC-MS (Method K, gradient: 10-50% B over 3 min): Rₜ=2.22 min; MS (ESIpos): m/z=1948.7 [M+H]⁺

### Example 1-D Monomer-²²⁷Th

### (3S,10S,14S)-1-[(1r,4S)-4-({2-[4-({2-[{3-[Bis(2-{[1-(Carboxymethyl)-3-(Hydroxy-kappaO)-6-methyl-2-(oxo-kappaO)-1,2-dihydropyridine-4-carbonyl]amino}ethyl)amino]propyl}(2-{[1-(carboxymethyl)-3-(hydroxy-kappaO)-6-methyl-2-(oxo-kappaO)-1,2-dihydropyridine-4-carbonyl]amino}ethyl)amino]ethyl}carbamoyl)-3-(hydroxy-kappaO)-6-methyl-2-(oxo-kappaO)pyridin-1(2H)-yl]acetamido}methyl)cyclohexyl]-3-[(naphthalen-2-yl)methyl]-1,4,12-trioxo-2,5,11,13-tetraazahexadecane-10,14,16-tricarboxylato(4−)(²²⁷Th)Thorium

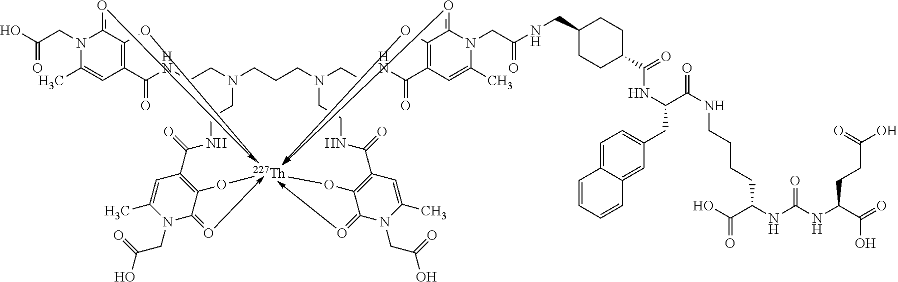

(3S,10S,14S)-1-[(1r,4S)-4-({2-[4-({2-[{3-[bis(2-{[1-(carboxymethyl)-3-hydroxy-6-methyl-2-oxo-1,2-dihydropyridine-4-carbonyl]amino}ethyl)amino]propyl}(2-{[1-(carboxymethyl)-3-hydroxy-6-methyl-2-oxo-1,2-dihydropyridine-4-carbonyl]amino}ethyl)amino]ethyl}carbamoyl)-3-hydroxy-6-methyl-2-oxopyridin-1(2H)-yl]acetamido}methyl)cyclohexyl]-3-[(naphthalen-2-yl)methyl]-1,4,12-trioxo-2,5,11,13-tetraazahexadecane-10,14,16-tricarboxylic acid (5.4 μg) dissolved in 186 μL 30 mM citrate buffer (pH 5.5) containing 0.5 mg/mL pABA was mixed with thorium-227 in 0.5 M HCl (2 μL) at 0.3 MBq/nmol specific activity and RAC of 2.9 MBq/mL. 0.1M carbonate buffer pH 9 (140 μL) was added and mixture incubated for 60 min. The labelling efficiency was determined to be 99% by iTLC.

### Example 1-E Dimer

### (3S,10S,14S,3'S,10'S,14'S)-1,1′-(propane-1,3-diylbis{[(2-{[1-(carboxymethyl)-3-hydroxy-6-methyl-2-oxo-1,2-dihydropyridine-4-carbonyl]amino}ethyl)azanediyl]ethane-2,1-diylcarbamoyl(3-hydroxy-6-methyl-2-oxopyridine-4,1(2H)-diyl)(1-oxoethane-2,1-diyl)azanediylmethylene(1r,4S)cyclohexane-4,1-diyl})bis{3-[(naphthalen-2-yl)methyl]-1,4,12-trioxo-2,5,11,13-tetraazahexadecane-10,14,16-tricarboxylic acid}

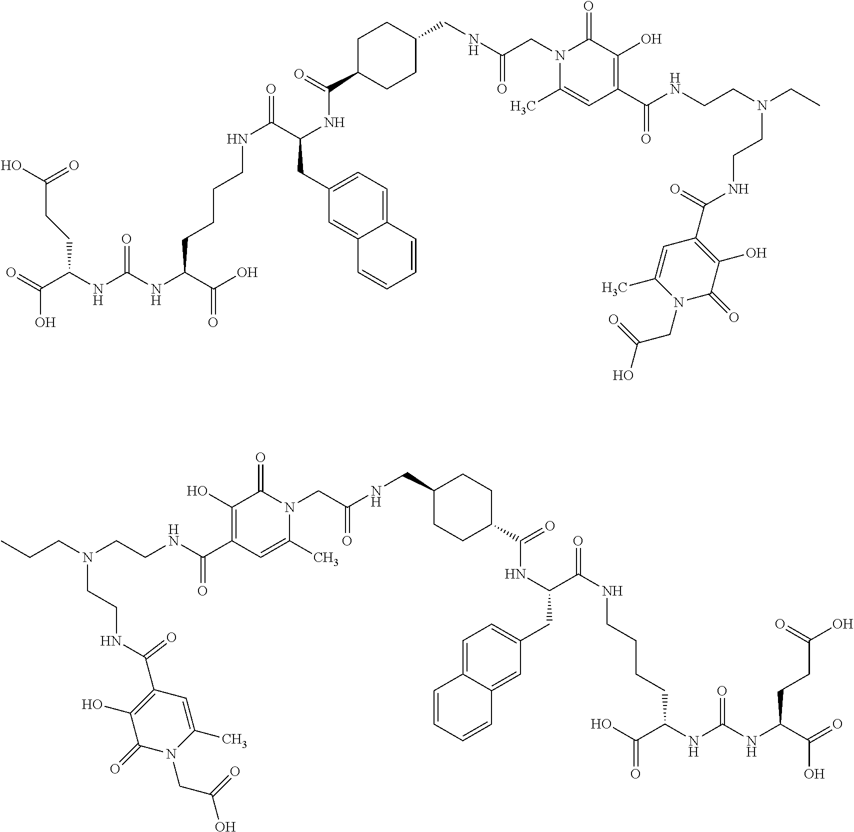

2,2′,2″,2′″-(propane-1,3-diylbis{nitrilobis[(ethane-2,1-diyl)carbamoyl(3-hydroxy-6-methyl-2-oxopyridine-4,1(2H)-diyl)]})tetraacetic acid (5.70 mg, 5.26 μmol) in 570 μL NMP and PyAOP (2.7 mg, 5.26 μmol) in 270 μL NMP are mixed in a vial. DIPEA (4.6 μL, 26 μmol) is added. (3S,10S,14S)-1-[(1r,4S)-4-(aminomethyl)cyclohexyl]-3-[(naphthalen-2-yl)methyl]-1,4,12-trioxo-2,5,11,13-tetraazahexadecane-10,14,16-tricarboxylic acid (5.18 mg, 7.89 μmol) in 520 μL NMP is added. Reaction mix is diluted with 10% ACN/water/0.1% TFA (7 mL) and products purified by preparative HPLC (RP-HPLC using Äkta pure system (ALG-106) Column: Phenomenex Luna 5 μm C18(2) 100 Å, 250×21.2 mm Mobile phase: Water/0.1% TFA; ACN Gradient: 10-50% B over 40 min Flow: 10 mL/min Detection: UV 280/335 nm tR product: 31 min tR by-product: 37 min) affording 3.60 mg (95% purity, 28% yield) of the target compound.

LC-MS (Method K, gradient: 10-50% B over 3 min): Rₜ=2.27 min; MS (ESIpos): m/z=1179.3 [M+2H]²⁺

### Example 1-F Dimer-²²⁷Th

### (3S,10S,14S,3'S,10'S,14'S)-1,1′-(propane-1,3-diylbis{[(2-{[1-(carboxymethyl)-3-(hydroxy-kappaO)-6-methyl-2-(oxo-kappaO)-1,2-dihydropyridine-4-carbonyl]amino}ethyl)azanediyl]ethane-2,1-diylcarbamoyl[3-(hydroxy-kappaO)-6-methyl-2-(oxo-kappaO)pyridine-4,1(2H)-diyl](1-oxoethane-2,1-diyl)azanediylmethylene(1r,4S)cyclohexane-4,1-diyl})bis{3-[(naphthalen-2-yl)methyl]-1,4,12-trioxo-2,5,11,13-tetraazahexadecane-10,14,16-tricarboxylato}(4−)(²²⁷Th)Thorium

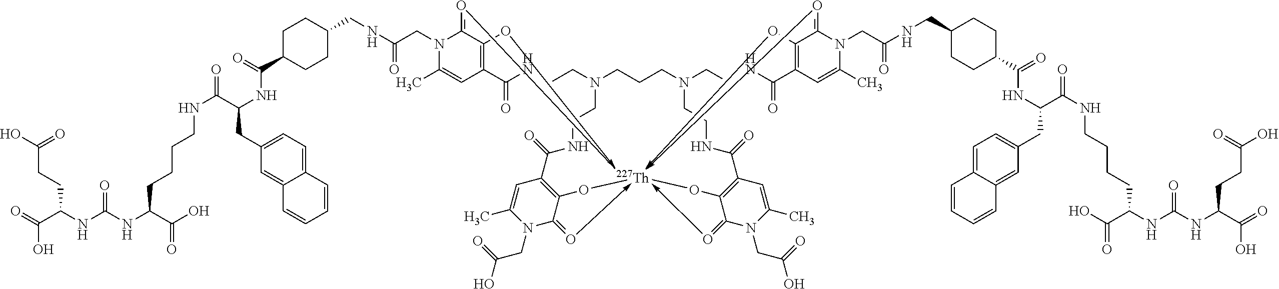

(3S,10S,14S,3'S,10'S,14'S)-1,1′-(propane-1,3-diylbis{[(2-{[1-(carboxymethyl)-3-hydroxy-6-methyl-2-oxo-1,2-dihydropyridine-4-carbonyl]amino}ethyl)azanediyl]ethane-2,1-diylcarbamoyl(3-hydroxy-6-methyl-2-oxopyridine-4,1(2H)-diyl)(1-oxoethane-2,1-diyl)azanediylmethylene(1r,4S)cyclohexane-4,1-diyl})bis{3-[(naphthalen-2-yl)methyl]-1,4,12-trioxo-2,5,11,13-tetraazahexadecane-10,14,16-tricarboxylic acid} (8 μg) dissolved in 20 μL 30 mM citrate buffer (pH 5.5) containing 0.5 mg/mL pABA was mixed with thorium-227 in 0.5 M HCl (2 μL) at 0.3 MBq/nmol specific activity and RAC of 5.9 MBq/mL. 0.1M carbonate buffer pH 9 (150 μL) was added and mixture incubated for 60 min. The labelling efficiency was determined to be 99% by iTLC.

Intermediate 7, Trimer

### {4-[(2-{(3-{bis[2-({1-[2-({[(1S,4r)-4-{[(7S,11S,18S)-7,11-bis(tert-butoxycarbonyl)-2,2-dimethyl-19-(naphthalen-2-yl)-4,9,17-trioxo-3-oxa-8,10,16-triazanonadecan-18-yl]carbamoyl}cyclohexyl]methyl}amino)-2-oxoethyl]-3-hydroxy-6-methyl-2-oxo-1,2-dihydropyridine-4-carbonyl}amino)ethyl]amino}propyl)[2-({1-[2-({[(1S,4r)-4-{[(7S,11S,18S)-7,11-bis(tert-butoxycarbonyl)-2,2-dimethyl-19-(naphthalen-2-yl)-4,9,17-trioxo-3-oxa-8,10,16-triazanonadecan-18-yl]carbamoyl}cyclohexyl]methyl}amino)-2-oxoethyl]-3-hydroxy-6-methyl-2-oxo-1,2-dihydropyridine-4-carbonyl}amino)ethyl]amino}ethyl)carbamoyl]-3-hydroxy-6-methyl-2-oxopyridin-1(2H)-yl}acetic acid

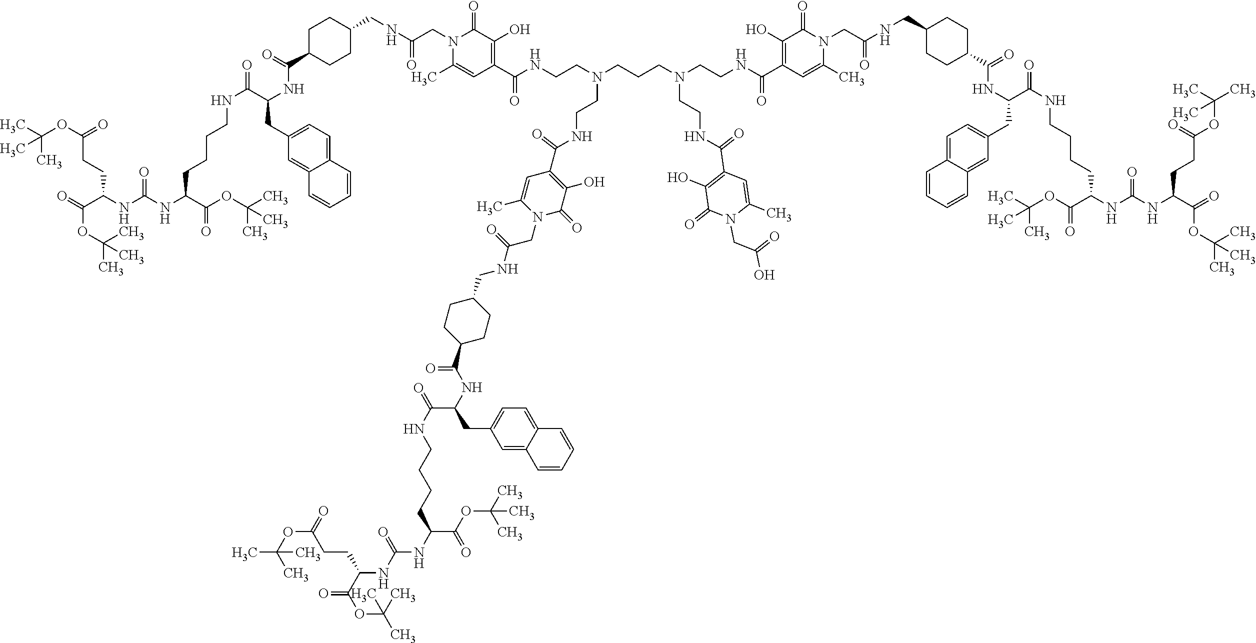

2,2′,2″,2′″-(propane-1,3-diylbis{nitrilobis[(ethane-2,1-diyl)carbamoyl(3-hydroxy-6-methyl-2-oxopyridine-4,1(2H)-diyl)]})tetraacetic acid (5 mg, 4.6 μmol) and tri-tert-butyl (3S,10S,14S)-1-[(1r,4S)-4-(aminomethyl)cyclohexyl]-3-[(naphthalen-2-yl)methyl]-1,4,12-trioxo-2,5,11,13-tetraazahexadecane-10,14,16-tricarboxylate (15.2 mg, 18.5 μmol) are dissolved in NMP (1.0 mL), DIPEA (8.0 μL) is added, then PyAOP (9.6 mg, 18.5 μmol) in NMP (960 μL) is added. Reaction mix is diluted with 50% ACN/water/0.1% TFA (6 mL) and the products purified by preparative HPLC (Äkta pure system, column: Phenomenex Luna 5 μm C18(2) 100 Å, 250×21.2 mm Mobile phase: Water/0.1% TFA; ACN Gradient: 50-100% B over 40 min Flow: 10 mL/min Detection: UV 280/335 nm, tR: 33 min) affording 2.5 mg of the target compound.

LC-MS (Method K, gradient: 50-100% B over 3 min): Rₜ=2.02 min; MS (ESIpos): m/z=1750.5 [M+2H]²⁺

### Example 1-G Trimer

### (3S,10S,14S,3'S,10'S,14'S)-1,1′-{[(3-{[2-({1-[2-({[(1S,4r)-4-{[(2S)-1-{[(5S)-5-carboxy-5-({[(1S)-1,3-dicarboxypropyl]carbamoyl}amino)pentyl]amino}-3-(naphthalen-2-yl)-1-oxopropan-2-yl]carbamoyl}cyclohexyl]methyl}amino)-2-oxoethyl]-3-hydroxy-6-methyl-2-oxopyridine-4(2H)-carbonyl}amino)ethyl](2-{[1-(carboxymethyl)-3-hydroxy-6-methyl-2-oxo-1,2-dihydropyridine-4-carbonyl]amino}ethyl)amino}propyl)azanediyl]bis[(ethane-2,1-diyl)carbamoyl(3-hydroxy-6-methyl-2-oxopyridine-4,1(2H)-diyl)(1-oxoethane-2,1-diyl)azanediylmethylene(1r,4S)cyclohexane-4,1-diyl]}bis{3-[(naphthalen-2-yl)methyl]-1,4,12-trioxo-2,5,11,13-tetraazahexadecane-10,14,16-tricarboxylic acid}

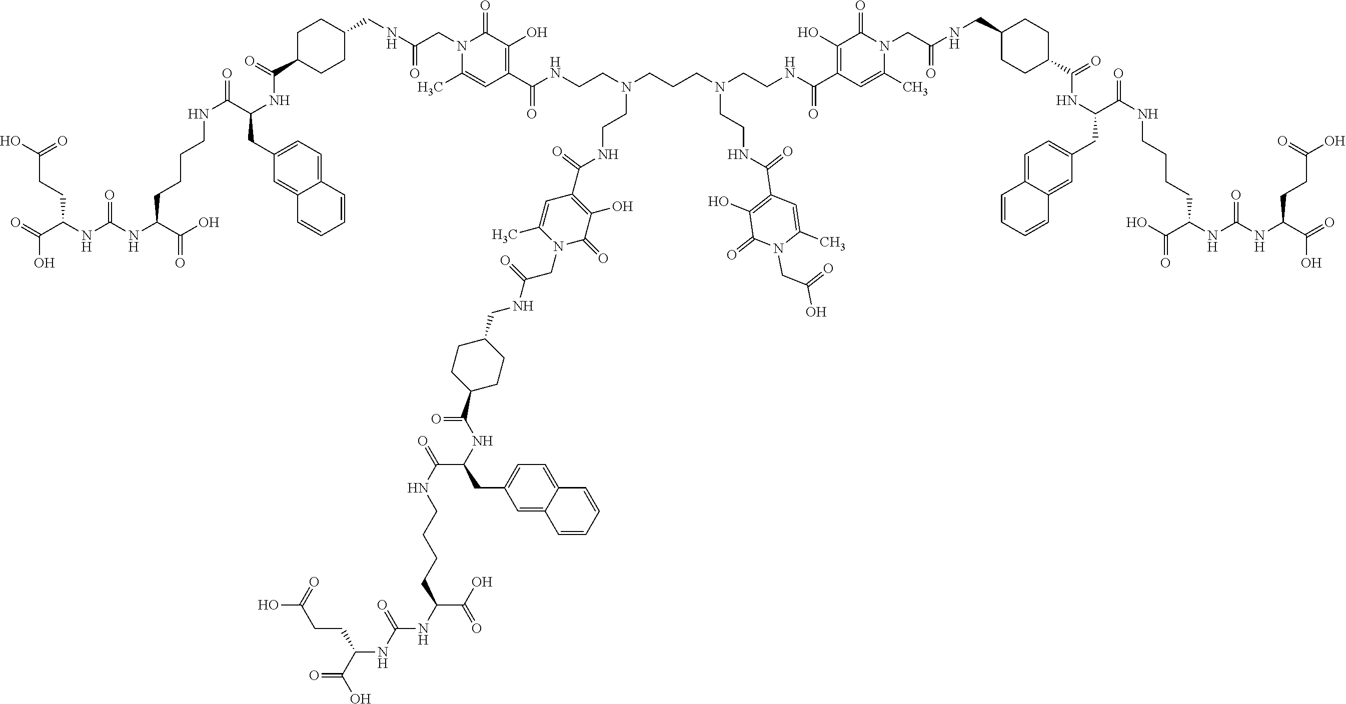 [SMILES: Cc1cc(C(=O)NCCN(CCCN(CCNC(=O)c2cc(C)n(CC(=O)NC[C@H]3CC[C@H](C(=O)N[C@@H](Cc4ccc5ccccc5c4)C(=O)NCCCC[C@H](NC(=O)N[C@@H](CCC(=O)O)C(=O)O)C(=O)O)CC3)c(=O)c2O)CCNC(=O)c2cc(C)n(CC(=O)NC[C@H]3CC[C@H](C(=O)N[C@@H](Cc4ccc5ccccc5c4)C(=O)NCCCC[C@H](NC(=O)N[C@@H](CCC(=O)O)C(=O)O)C(=O)O)CC3)c(=O)c2O)CCNC(=O)c2cc(C)n(CC(=O)NC[C@H]3CC[C@H](C(=O)N[C@@H](Cc4ccc5ccccc5c4)C(=O)NCCCC[C@H](NC(=O)N[C@@H](CCC(=O)O)C(=O)O)C(=O)O)CC3)c(=O)c2O)c(O)c(=O)n1CC(=O)O]

{4-[(2-{(3-{bis[2-({1-[2-({[(1S,4r)-4-{[(7S,11S,18S)-7,11-bis(tert-butoxycarbonyl)-2,2-dimethyl-19-(naphthalen-2-yl)-4,9,17-trioxo-3-oxa-8,10,16-triazanonadecan-18-yl]carbamoyl}cyclohexyl]methyl}amino)-2-oxoethyl]-3-hydroxy-6-methyl-2-oxo-1,2-dihydropyridine-4-carbonyl}amino)ethyl]amino}propyl)[2-({1-[2-({[(1S,4r)-4-{[(7S,11S,18S)-7,11-bis(tert-butoxycarbonyl)-2,2-dimethyl-19-(naphthalen-2-yl)-4,9,17-trioxo-3-oxa-8,10,16-triazanonadecan-18-yl]carbamoyl}cyclohexyl]methyl}amino)-2-oxoethyl]-3-hydroxy-6-methyl-2-oxo-1,2-dihydropyridine-4-carbonyl}amino)ethyl]amino}ethyl)carbamoyl]-3-hydroxy-6-methyl-2-oxopyridin-1(2H)-yl}acetic acid (2.50 mg, 0.714 μmol) is treated with 90% TFA in water (0.5 mL) for 2 hrs. Water (18 mL) is added and reaction mixture is lyophilised affording 2.10 mg (95% purity, 93% yield) of the target compound.

LC-MS (Method K, gradient: 10-70% B over 3 min): Rₜ=1.84 min; MS (ESIpos): m/z=1498.1 [M+2H]²⁺

### Example 1-H Trimer-²²⁷Th

### (3S,10S,14S,3'S,10'S,14'S)-1,1′-{[(3-{[2-({1-[2-({[(1S,4r)-4-{[(2S)-1-{[(5S)-5-carboxy-5-({[(1S)-1,3-dicarboxypropyl]carbamoyl}amino)pentyl]amino}-3-(naphthalen-2-yl)-1-oxopropan-2-yl]carbamoyl}cyclohexyl]methyl}amino)-2-oxoethyl]-3-(hydroxy-kappaO)-6-methyl-2-oxopyridine-4(2H)-carbonyl}amino)ethyl](2-{[1-(carboxymethyl)-3-(hydroxy-kappaO)-6-methyl-2-oxo-1,2-dihydropyridine-4-carbonyl]amino}ethyl)amino}propyl)azanediyl]bis[(ethane-2,1-diyl)carbamoyl[3-(hydroxy-kappaO)-6-methyl-2-oxopyridine-4,1(2H)-diyl](1-oxoethane-2,1-diyl)azanediylmethylene(1r,4S)cyclohexane-4,1-diyl]}bis{3-[(naphthalen-2-yl)methyl]-1,4,12-trioxo-2,5,11,13-tetraazahexadecane-10,14,16-tricarboxylato}(4−)(²²⁷Th)Thorium

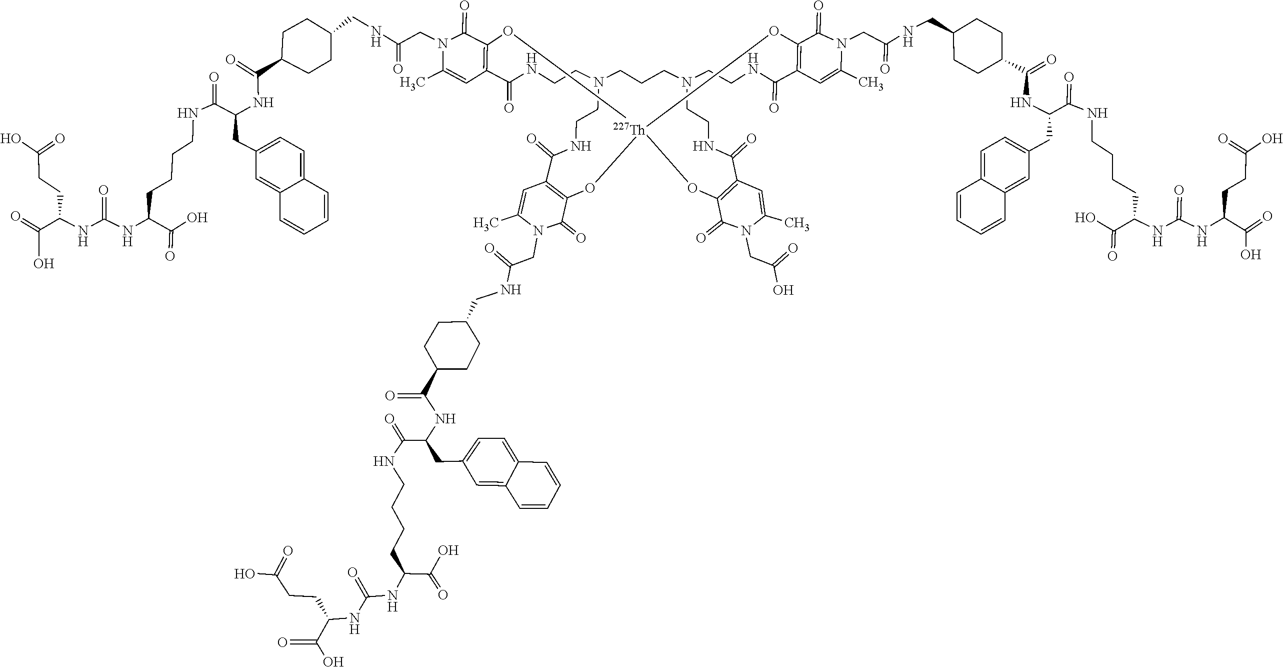

(3S,10S,14S,3'S,10'S,14'S)-1,1′-{[(3-{[2-({1-[2-({[(1S,4r)-4-{[(2S)-1-{[(5S)-5- carboxy-5-({[(1S)-1,3-dicarboxypropyl]carbamoyl}amino)pentyl]amino}-3-(naphthalen-2-yl)-1-oxopropan-2-yl]carbamoyl}cyclohexyl]methyl}amino)-2-oxoethyl]-3-hydroxy-6-methyl-2-oxopyridine-4(2H)-carbonyl}amino)ethyl](2-{[1-(carboxymethyl)-3-hydroxy-6-methyl-2-oxo-1,2-dihydropyridine-4-carbonyl]amino}ethyl)amino}propyl)azanediyl]bis[(ethane-2,1-diyl)carbamoyl(3-hydroxy-6-methyl-2-oxopyridine-4,1(2H)-diyl)(1-oxoethane-2,1-diyl)azanediylmethylene(1r,4S)cyclohexane-4,1-diyl]}bis{3-[(naphthalen-2-yl)methyl]-1,4,12-trioxo-2,5,11,13-tetraazahexadecane-10,14,16-tricarboxylic acid} (8 μg) dissolved in 159 μL 30 mM citrate buffer (pH 5.5) containing 0.5 mg/mL pABA was mixed with thorium-227 in 0.5 M HCl (2 μL) at 0.3 MBq/nmol specific activity and RAC of 4.6 MBq/mL. 0.1M carbonate buffer pH 9 (16 μL) was added and mixture incubated for 60 min. The labelling efficiency was determined to be 97% by iTLC.

### Example 1-1 Tetramer

### (2S)-2-[[(1S)-5-[[(2S)-2-[[4-[[[2-[4-[2-[3-[bis[2-[[1-[2-[[4-[[(1S)-2-[[(5S)-5-carboxy-5-[[(1S)-1,3-dicarboxypropyl]carbamoylamino]pentyl]amino]-1-(2-naphthylmethyl)-2-oxo-ethyl]carbamoyl]cyclohexyl]methylamino]-2-oxo-ethyl]-3-hydroxy-6-methyl-2-oxo-pyridine-4-carbonyl]amino]ethyl]amino]propyl-[2-[[1-[2-[[4-[[(1S)-2-[[(5S)-5-carboxy-5-[[(1S)-1,3-dicarboxypropyl]carbamoylamino]pentyl]amino]-1-(2-naphthylmethyl)-2-oxo-ethyl]carbamoyl]cyclohexyl]methylamino]-2-oxo-ethyl]-3-hydroxy-6-methyl-2-oxo-pyridine-4-carbonyl]amino]ethyl]amino]ethylcarbamoyl]-3-hydroxy-6-methyl-2-oxo-1-pyridyl]acetyl]amino]methyl]cyclohexanecarbonyl]amino]-3-(2-naphthyl)propanoyl]amino]-1-carboxy-pentyl]carbamoylamino]pentanedioic acid

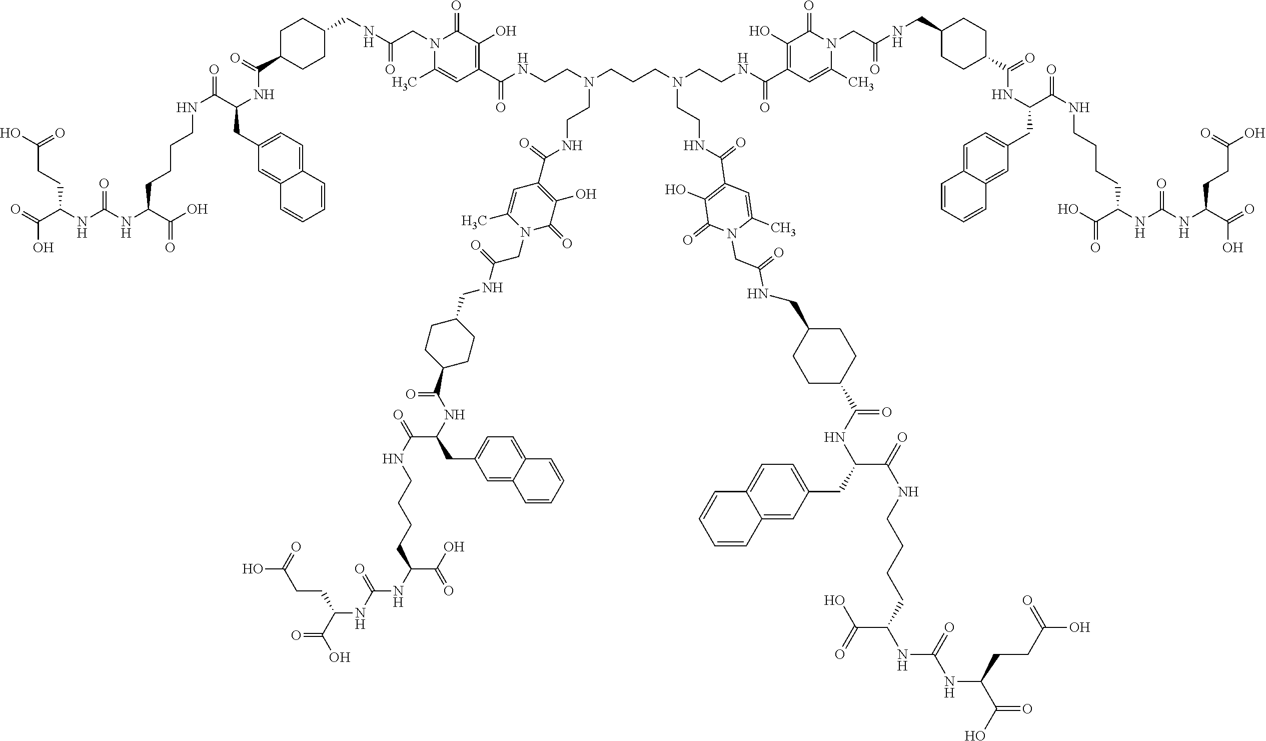

2,2′,2″,2′″-(propane-1,3-diylbis{nitrilobis[(ethane-2,1-diyl)carbamoyl(3-hydroxy-6-methyl-2-oxopyridine-4,1(2H)-diyl)]})tetraacetic acid (7.40 mg, 6.83 μmol) and PyAOP (14.3 mg, 27.3 μmol) are dissolved in NMP (1 mL), DIPEA (11.9 μL, 68 μmol) is added. (3S,10S,14S)-1-[(1r,4S)-4-(aminomethyl)cyclohexyl]-3-[(naphthalen-2-yl)methyl]-1,4,12-trioxo-2,5,11,13-tetraazahexadecane-10,14,16-tricarboxylic acid (31.4 mg, 47.8 μmol) is added. Reaction mix is quenched with 20% ACN/water (8 mL) and the product purified by preparative HPLC (RP-HPLC using Äkta pure system, Column: Phenomenex Luna 5 μm C18(2) 100 Å, 250×21.2 mm Mobile phase: Water/0.1% TFA; ACN Gradient: 20-60% B over 40 min Flow: 10 mL/min Detection: UV 280/335 nm tR: 34 min) affording 2.50 mg (95% purity, 10% yield) of the target compound.

LC-MS (Method K, gradient: 20-60% B over 3 min): Rₜ=1.89 min; MS (ESIpos): m/z=1816.8 [M+2H]²⁺

### Example 1-J Tetramer-²²⁷Th

### (2S)-2-[[(1S)-5-[[(2S)-2-[[4-[[[2-[4-[2-[3-[bis[2-[[1-[2-[[4-[[(1S)-2-[[(5S)-5-carboxy-5-[[(1S)-1,3-dicarboxypropyl]carbamoylamino]pentyl]amino]-1-(2-naphthylmethyl)-2-oxo-ethyl]carbamoyl]cyclohexyl]methylamino]-2-oxo-ethyl]-3-(hydroxy-kappaO)-6-methyl-2-oxo-pyridine-4-carbonyl]amino]ethyl]amino]propyl-[2-[[1-[2-[[4-[[(1S)-2-[[(5S)-5-carboxy-5-[[(1S)-1,3-dicarboxypropyl]carbamoylamino]pentyl]amino]-1-(2-naphthylmethyl)-2-oxo-ethyl]carbamoyl]cyclohexyl]methylamino]-2-oxo-ethyl]-3-(hydroxy-kappaO)-6-methyl-2-oxo-pyridine-4-carbonyl]amino]ethyl]amino]ethylcarbamoyl]-3-(hydroxyl-kappaO)-6-methyl-2-oxo-1-pyridyl]acetyl]amino]methyl]cyclohexanecarbonyl]amino]-3-(2-naphthyl)propanoyl]amino]-1-carboxylato-pentyl]carbamoylamino]pentanedioate(4−)(²²⁷Th)Thorium

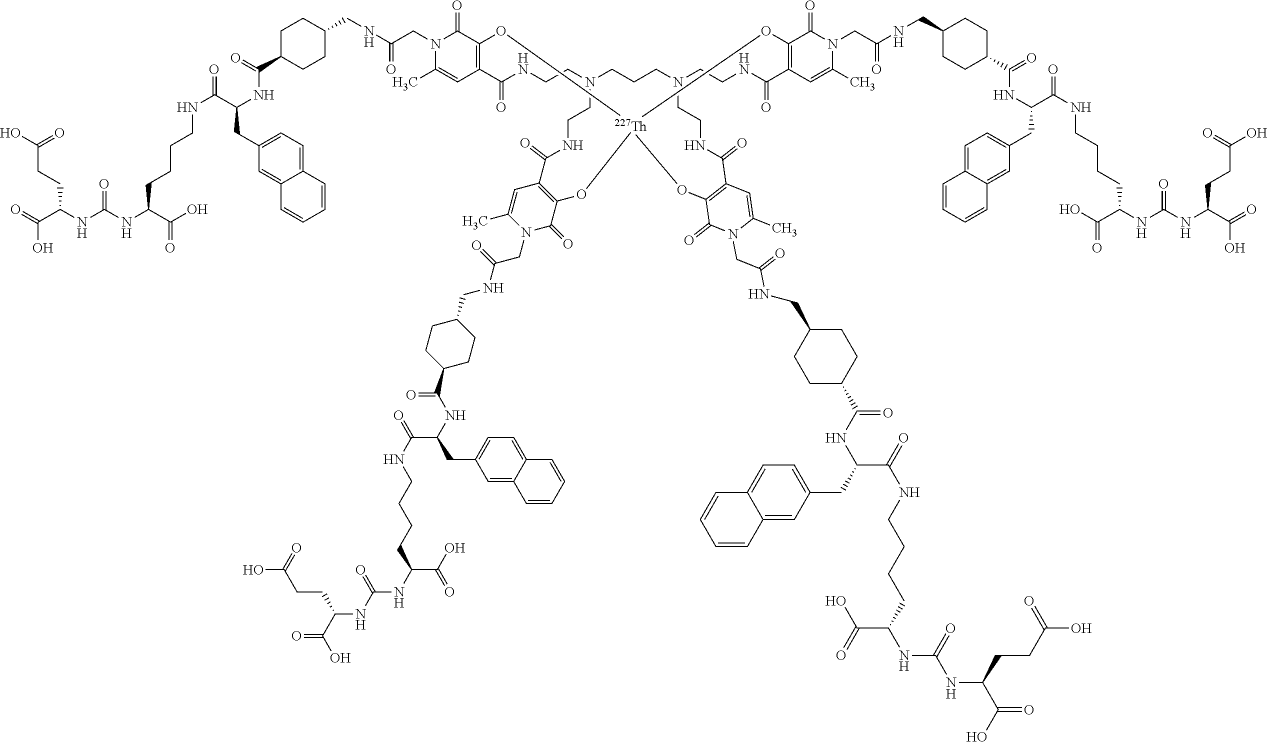

(2S)-2-[[(1S)-5-[[(2S)-2-[[4-[[[2-[4-[2-[3-[bis[2-[[1-[2-[[4-[[(1S)-2-[[(5S)-5-carboxy-5-[[(1S)-1,3-dicarboxypropyl]carbamoylamino]pentyl]amino]-1-(2-naphthylmethyl)-2-oxo-ethyl]carbamoyl]cyclohexyl]methylamino]-2-oxo-ethyl]-3-hydroxy-6-methyl-2-oxo-pyridine-4-carbonyl]amino]ethyl]amino]propyl-[2-[[1-[2-[[4-[[(1S)-2-[[(5S)-5-carboxy-5-[[(1S)-1,3-dicarboxypropyl]carbamoylamino]pentyl]amino]-1-(2-naphthylmethyl)-2-oxo-ethyl]carbamoyl]cyclohexyl]methylamino]-2-oxo-ethyl]-3-hydroxy-6-methyl-2-oxo-pyridine-4-carbonyl]amino]ethyl]amino]ethylcarbamoyl]-3-hydroxy-6-methyl-2-oxo-1-pyridyl]acetyl]amino]methyl]cyclohexanecarbonyl]amino]-3-(2-naphthyl)propanoyl]amino]-1-carboxy-pentyl]carbamoylamino]pentanedioic acid (7 μg) dissolved in 108 μL 30 mM citrate buffer (pH 5.5) containing 0.5 mg/mL pABA was mixed with thorium-227 in 0.5 M HCl (2 μL) at 0.3 MBq/nmol specific activity and RAC of 5.7 MBq/mL. 0.1M carbonate buffer pH 9 (11 μL) was added and mixture incubated for 2 hrs. The labelling efficiency was determined to be 98% by iTLC.

### Example 1-K

### (3S,10S,14S)-1-{(1 r,4S)-4-[(2-{4-[(7,10-bis{3-[(2-{[1-(carboxymethyl)-3-hydroxy-6-methyl-2-oxo-1,2-dihydropyridine-4-carbonyl]amino}ethyl)amino]-3-oxopropyl}-18-[1-(carboxymethyl)-3-hydroxy-6-methyl-2-oxo-1,2-dihydropyridin-4-yl]-4,13,18-trioxo-3,7,10,14,17-pentaazaoctadecan-1-yl)carbamoyl]-3-hydroxy-6-methyl-2-oxopyridin-1(2H)-yl}acetamido)methyl]cyclohexyl}-3-[(naphthalen-2-yl)methyl]-1,4,12-trioxo-2,5,11,13-tetraazahexadecane-10,14,16-tricarboxylic acid

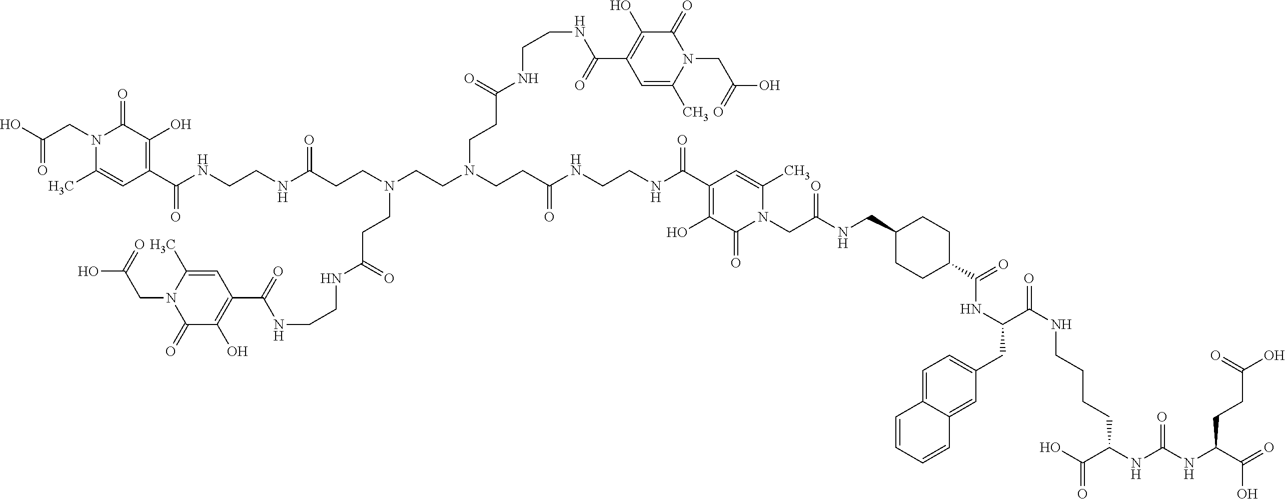 [SMILES: Cc1cc(C(=O)NCCNC(=O)CCN(CCC(=O)NCCNC(=O)c2cc(C)n(CC(=O)O)c(=O)c2O)CCN(CCC(=O)NCCNC(=O)c2cc(C)n(CC(=O)O)c(=O)c2O)CCC(=O)NCCNC(=O)c2cc(C)n(CC(=O)NC[C@H]3CC[C@H](C(=O)N[C@@H](Cc4ccc5ccccc5c4)C(=O)NCCCC[C@H](NC(=O)N[C@@H](CCC(=O)O)C(=O)O)C(=O)O)CC3)c(=O)c2O)c(O)c(=O)n1CC(=O)O]

{4-[(7,10-bis{3-[(2-{[1-(carboxymethyl)-3-hydroxy-6-methyl-2-oxo-1,2-dihydropyridine-4-carbonyl]amino}ethyl)amino]-3-oxopropyl}-18-[1-(carboxymethyl)-3-hydroxy-6-methyl-2-oxo-1,2-dihydropyridin-4-yl]-4,13,18-trioxo-3,7,10,14,17-pentaazaoctadecan-1-yl)carbamoyl]-3-hydroxy-6-methyl-2-oxopyridin-1(2H)-yl}acetic acid (2.10 mg, 1.55 μmol) in 210 μL NMP, 2,4,6-trimethylpyridine (2.1 μL) and TSTU (0.5 mg in 50 μL NMP) are mixed in a vial. (3S,10S,14S)-1-[(1r,4S)-4-(aminomethyl)cyclohexyl]-3-[(naphthalen-2-yl)methyl]-1,4,12-trioxo-2,5,11,13-tetraazahexadecane-10,14,16-tricarboxylic acid (1.53 mg, 2.33 μmol) is added. Reaction mix is diluted with water/0.1% TFA (4 mL) and the product purified by preparative HPLC (RP-HPLC using Äkta pure system (ALG-106) Column: Phenomenex Luna 5 μm C18(2) 100 Å, 250×21.2 mm Mobile phase: Water/0.1% TFA; ACN Gradient: 10-50% B over 40 min Flow: 10 mL/min Detection: UV 280/335 nm, tR product: 32 min) affording 2.00 mg (95% purity, 61% yield) of the target compound.

LC-MS (Method K, gradient: 10-50% B over 3 min): Rₜ=1.78 min; MS (ESIpos): m/z=1990.9 [M+H]⁺

### Example 1-L-²²⁷Th

### (3S,10S,14S)-1-{(1 r,4S)-4-[(2-{4-[(7,10-bis{3-[(2-{[1-(carboxymethyl)-3-(hydroxy-kappaO)-6-methyl-2-(oxo-kappaO)-1,2-dihydropyridine-4-carbonyl]amino}ethyl)amino]-3-oxopropyl}-18-[1-(carboxymethyl)-3-(hydroxy-kappaO)-6-methyl-2-(oxo-kappaO)-1,2-dihydropyridin-4-yl]-4,13,18-trioxo-3,7,10,14,17-pentaazaoctadecan-1-yl)carbamoyl]-3-(hydroxy-kappaO)-6-methyl-2-(oxo-kappaO)pyridin-1(2H)-yl}acetamido)methyl]cyclohexyl}-3-[(naphthalen-2-yl)methyl]-1,4,12-trioxo-2,5,11,13-tetraazahexadecane-10,14,16-tricarboxylato(4−)(²²⁷Th)Thorium

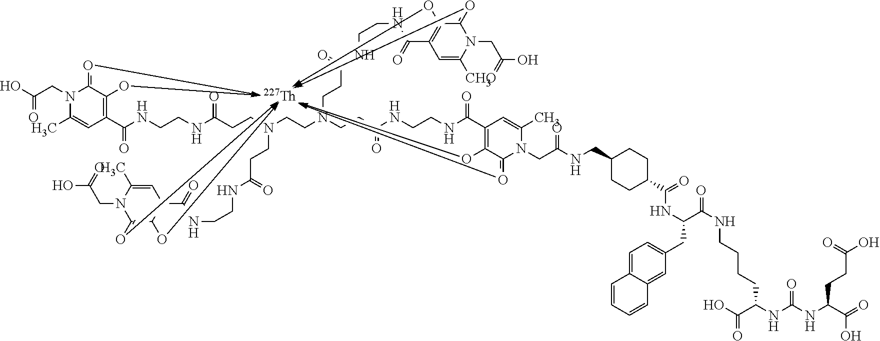

(3S,10S,14S)-1-{(1r,4S)-4-[(2-{4-[(7,10-bis{3-[(2-{[1-(carboxymethyl)-3-hydroxy-6-methyl-2-oxo-1,2-dihydropyridine-4-carbonyl]amino}ethyl)amino]-3-oxopropyl}-18-[1-(carboxymethyl)-3-hydroxy-6-methyl-2-oxo-1,2-dihydropyridin-4-yl]-4,13,18-trioxo-3,7,10,14,17-pentaazaoctadecan-1-yl)carbamoyl]-3-hydroxy-6-methyl-2-oxopyridin-1(2H)-yl}acetamido)methyl]cyclohexyl}-3-[(naphthalen-2-yl)methyl]-1,4,12-trioxo-2,5,11,13-tetraazahexadecane-10,14,16-tricarboxylic acid (4.9 μg) dissolved in 149 μL 30 mM citrate buffer (pH 5.5) containing 0.5 mg/mL pABA was mixed with thorium-227 in 0.5 M HCl (2 μL) at 0.375 MBq/nmol specific activity and RAC of 5.6 MBq/mL. 1M HEPES buffer pH 7.5 (15 μL) was added and mixture incubated for 30 min. The labelling efficiency was determined to be 65% by iTLC.

Intermediate 8

### (3S,10S,14S)-1-[(1r,4S)-4-(47,47-dimethyl-3,7,45-trioxo-5,11,14,17,20,23,26,29,32,35,38,41,46-tridecaoxa-2,8,44-triazaoctatetracontan-1-yl)cyclohexyl]-3-[(naphthalen-2-yl)methyl]-1,4,12-trioxo-2,5,11,13-tetraazahexadecane-10,14,16-tricarboxylic acid

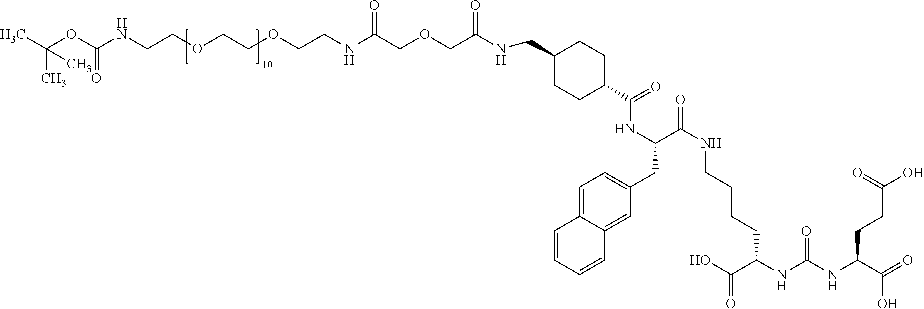

2,2-dimethyl-4,42-dioxo-3,8,11,14,17,20,23,26,29,32,35,38,44-tridecaoxa-5,41-diazahexatetracontan-46-oic acid (5.00 mg, 6.57 μmol) (5 mg in 500 μL NMP) and PyAOP (3.4 mg, 6.6 μmol) in 340 μL NMP are mixed in a vial. N,N-diisopropylethylamine (2.3 μL) is added.

pH strip indicates neutral reaction mixture. N,N-diisopropylethylamine (1.0 μL, 19 μmol) is added. (3S,10S,14S)-1-[(1r,4S)-4-(aminomethyl)cyclohexyl]-3-[(naphthalen-2-yl)methyl]-1,4,12-trioxo-2,5,11,13-tetraazahexadecane-10,14,16-tricarboxylic acid (4.31 mg, 6.57 μmol) in 430 μL NMP is added. Purification by RP-HPLC (Äkta pure system Column: Phenomenex Luna 5 μm C18(2) 100 Å, 250×21.2 mm Mobile phase: Water/0.1% TFA; ACN Gradient: 20-50% B over 40 min Flow: 10 mL/min Detection: UV 280/276 nm tR product=40 min afforded 4.5 mg (49%) of the target compound.

LC-MS (Method K, gradient: 10-50% B over 3 min): Rₜ=2.29 min; MS (ESIpos): m/z=1398.7 [M+H]⁺

Intermediate 9

### (3S,10S,14S)-1-[(1r,4S)-4-(43-amino-3,7-dioxo-5,11,14,17,20,23,26,29,32,35,38,41-dodecaoxa-2,8-diazatritetracontan-1-yl)cyclohexyl]-3-[(naphthalen-2-yl)methyl]-1,4,12-trioxo-2,5,11,13-tetraazahexadecane-10,14,16-tricarboxylic acid

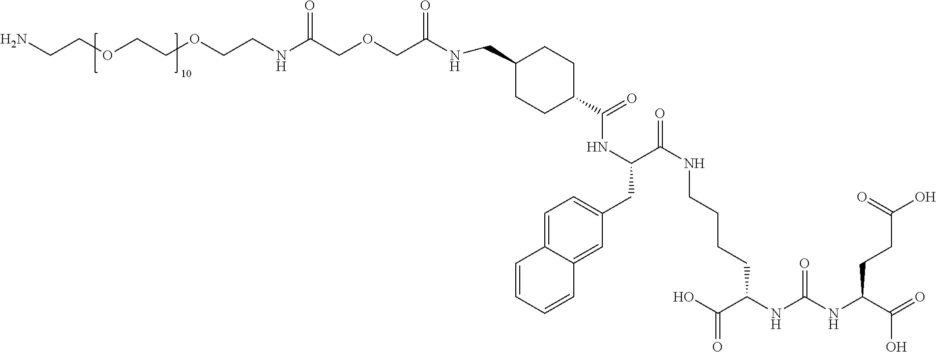

(3S,10S,14S)-1-[(1r,4S)-4-(47,47-dimethyl-3,7,45-trioxo-5,11,14,17,20,23,26,29,32,35,38,41,46-tridecaoxa-2,8,44-triazaoctatetracontan-1-yl)cyclohexyl]-3-[(naphthalen-2-yl)methyl]-1,4,12-trioxo-2,5,11,13-tetraazahexadecane-10,14,16-tricarboxylic acid (9.00 mg, 6.43 μmol) is treated with 95% TFA/water. TFA is removed by evaporation, water (10 mL) is added to the residue followed by lyophilisation affording 8.4 mg (100% yield) of the target compound.

LC-MS (Method K, gradient: 10-50% B over 3 min): Rₜ=1.8 min; MS (ESIpos): m/z=1298.7 [M+H]⁺

Intermediate 10a

### {2-[4-(3-[bis(2-{[3-hydroxy-1-(2-hydroxyethyl)-2-oxo-1,2-dihydropyridine-4-carbonyl]amino}ethyl)amino]-2-{[bis(2-{[3-hydroxy-1-(2-hydroxyethyl)-2-oxo-1,2-dihydropyridine-4-carbonyl]amino}ethyl)amino]methyl}propyl)anilino]-2-oxoethoxy}acetic acid

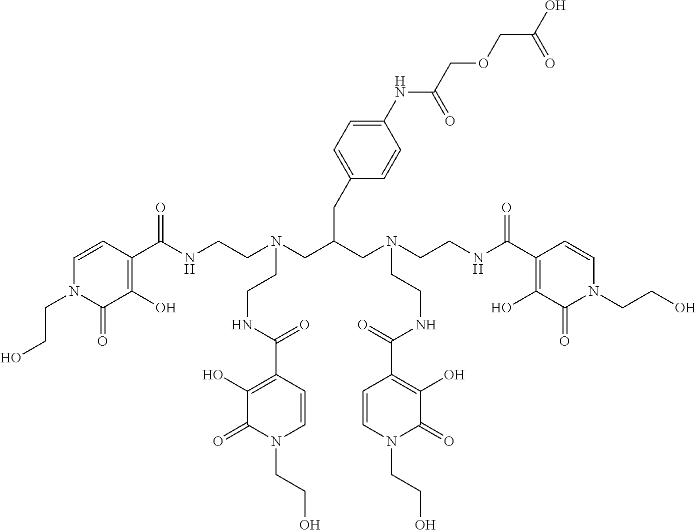 [SMILES: O=C(O)COCC(=O)Nc1ccc(CC(CN(CCNC(=O)c2ccn(CCO)c(=O)c2O)CCNC(=O)c2ccn(CCO)c(=O)c2O)CN(CCNC(=O)c2ccn(CCO)c(=O)c2O)CCNC(=O)c2ccn(CCO)c(=O)c2O)cc1]

To a solution of N,N′,N,N′-({2-[(4-aminophenyl)methyl]propane-1,3-diyl}bis[nitrilodi(ethane-2,1-diyl)])tetrakis[3-hydroxy-1-(2-hydroxyethyl)-2-oxo-1,2-dihydropyridine-4-carboxamide] (100 mg, 80% purity, 74.3 μmol) in NMP 01 mL) was added at r.t. 1,4-dioxane-2,6-dione (32.9 mg, 97% purity, 275 μmol) and 2,4,6-trimethylpiperidine (48 μl, 360 μmol). After stirring for 15 min at r.t. the mixture was diluted with 0.1% aqueous formic acid (3.5 mL), adjusted to pH 14 with 5N aq. NaOH and then adjusted to pH with TFA. The mixture was purified by preparative

LC-MS (Method 1): Rₜ=0.62 min; MS (ESIpos): m/z=1193 [M+H]⁺

¹H-NMR (400 MHz, DMSO-d6) δ[ppm]: 1.232 (0.45), 1.907 (0.45), 2.074 (0.90), 2.195 (0.65), 2.332 (2.65), 2.336 (1.16), 2.518 (16.00), 2.522 (10.97), 2.669 (3.68), 2.673 (2.71), 2.678 (1.23), 3.608 (4.06), 3.622 (8.58), 3.635 (4.77), 3.918 (3.87), 3.932 (6.58), 3.945 (3.29), 4.119 (4.52), 4.162 (3.68), 6.481 (5.16), 6.499 (5.16), 6.989 (1.55), 7.011 (1.68), 7.051 (6.77), 7.069 (6.13), 7.407 (2.26), 7.428 (1.87), 8.133 (2.00), 8.523 (1.35).

### Example 1-PEG

### (3S,10S,14S)-1-[(1r,4S)-4-{49-[4-(3-[bis(2-{[3-hydroxy-1-(2-hydroxyethyl)-2-oxo-1,2-dihydropyridine-4-carbonyl]amino}ethyl)amino]-2-{[bis(2-{[3-hydroxy-1-(2-hydroxyethyl)-2-oxo-1,2-dihydropyridine-4-carbonyl]amino}ethyl)amino]methyl}propyl)anilino]-3,7,45,49-tetraoxo-5,11,14,17,20,23,26,29,32,35,38,41,47-tridecaoxa-2,8,44-triazanonatetracontan-1-yl}cyclohexyl]-3-[(naphthalen-2-yl)methyl]-1,4,12-trioxo-2,5,11,13-tetraazahexadecane-10,14,16-tricarboxylic acid

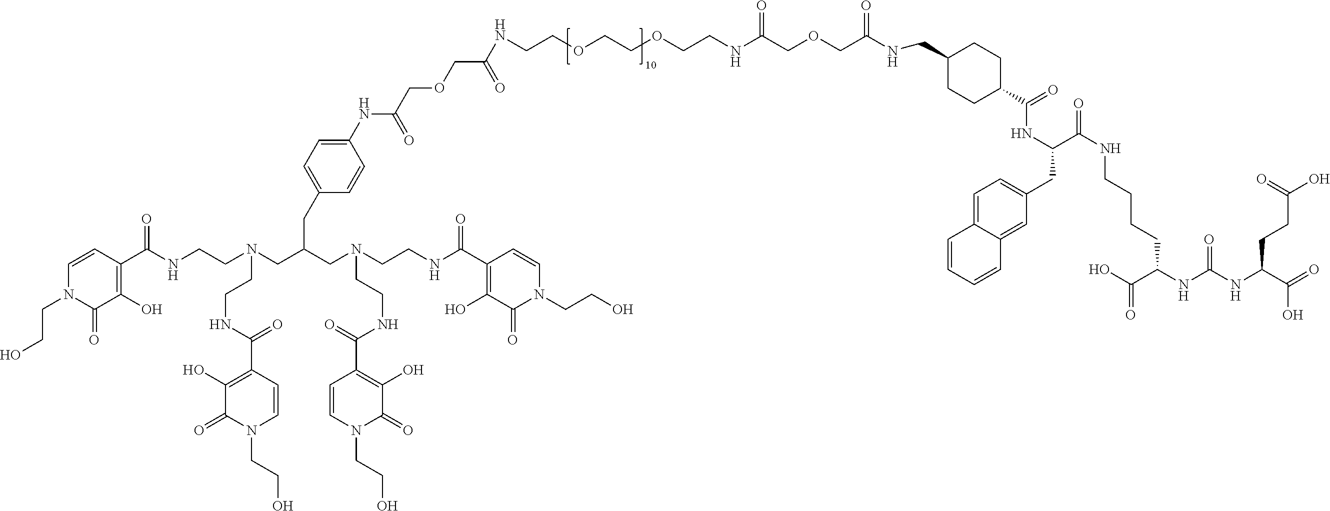

{2-[4-(3-[bis(2-{[3-hydroxy-1-(2-hydroxyethyl)-2-oxo-1,2-dihydropyridine-4-carbonyl]amino}ethyl)amino]-2-{[bis(2-{[3-hydroxy-1-(2-hydroxyethyl)-2-oxo-1,2-dihydropyridine-4-carbonyl]amino}ethyl)amino]methyl}propyl)anilino]-2-oxoethoxy}acetic acid (5.00 mg, 4.19 μmol) in 500 μL NMP and PyAOP (2.2 mg, 4.19 μmol) in 220 μL NMP are mixed in a vial. 2,4,6-Trimethylpyridine (2.8 μL, 42 μmol) is added, then (3S,10S,14S)-1-[(1r,4S)-4-(43-amino-3,7-dioxo-5,11,14,17,20,23,26,29,32,35,38,41-dodecaoxa-2,8-diazatritetracontan-1-yl)cyclohexyl]-3-[(naphthalen-2-yl)methyl]-1,4,12-trioxo-2,5,11,13-tetraazahexadecane-10,14,16-tricarboxylic acid (5.99 mg, 4.61 μmol) in 600 μL NMP is added. Reaction mixture is diluted with water/0.1% TFA (3.5 mL) and the product purified by preparative HPLC (RP-HPLC using Äkta pure system, Column: Phenomenex Luna 5 μm C18(2) 100 Å, 250×21.2 mm Mobile phase: Water/0.1% TFA; ACN Gradient: 10-50% B over 40 min Flow: 10 mL/min Detection: UV 280/335 nm, tR product=32 min) affording 2.00 mg (93% purity, 18% yield)

LC-MS (Method K, gradient: 10-50% B over 3 min): Rₜ=1.69 min; MS (ESIpos): m/z=1236.6 [M+H]⁺

### Example 1-PEG-²²⁷Th

### (3S,10S,14S)-1-[(1r,4S)-4-{49-[4-(3-[bis(2-{[3-(hydroxy-kappaO)-1-(2-hydroxyethyl)-2-(oxo-kappaO)-1,2-dihydropyridine-4-carbonyl]amino}ethyl)amino]-2-{[bis(2-{[3-(hydroxy-kappaO)-1-(2-hydroxyethyl)-2-(oxo-kappaO)-1,2-dihydropyridine-4-carbonyl]amino}ethyl)amino]methyl}propyl)anilino]-3,7,45,49-tetraoxo-5,11,14,17,20,23,26,29,32,35,38,41,47-tridecaoxa-2,8,44-triazanonatetracontan-1-yl}cyclohexyl]-3-[(naphthalen-2-yl)methyl]-1,4,12-trioxo-2,5,11,13-tetraazahexadecane-10,14,16-tricarboxylato(4−)(²²⁷Th)Thorium

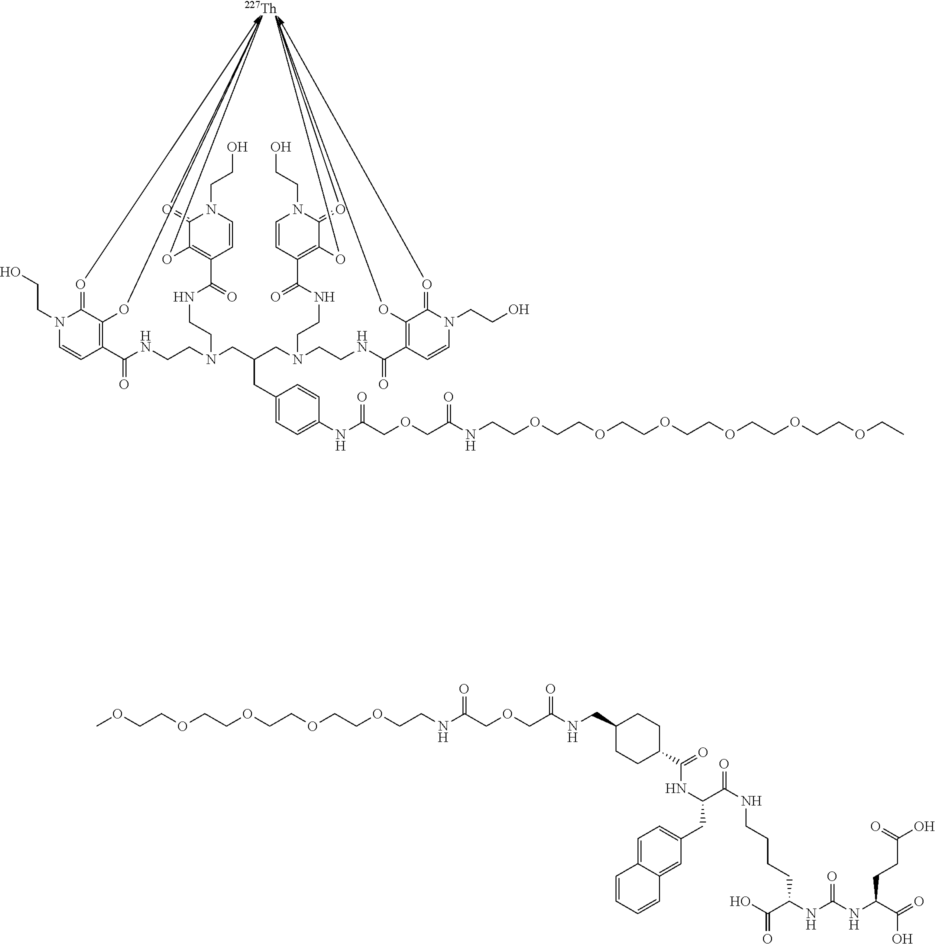

(3S,10S,14S)-1-[(1r,4S)-4-{49-[4-(3-[bis(2-{[3-hydroxy-1-(2-hydroxyethyl)-2-oxo-1,2-dihydropyridine-4-carbonyl]amino}ethyl)amino]-2-{[bis(2-{[3-hydroxy-1-(2-hydroxyethyl)-2-oxo-1,2-dihydropyridine-4-carbonyl]amino}ethyl)amino]methyl}propyl)anilino]-3,7,45,49-tetraoxo-5,11,14,17,20,23,26,29,32,35,38,41,47-tridecaoxa-2,8,44-triazanonatetracontan-1-yl}cyclohexyl]-3-[(naphthalen-2-yl)methyl]-1,4,12-trioxo-2,5,11,13-tetraazahexadecane-10,14,16-tricarboxylic acid (6.9 μg) dissolved in 167 μL 30 mM citrate buffer (pH 5.5) containing 0.5 mg/mL pABA was mixed with thorium-227 in 0.5 M HCl (2 μL) at 0.375 MBq/nmol specific activity and RAC of 4.8 MBq/mL. Ethanol (50 μL) was added and mixture incubated for 60 min. The labelling efficiency was determined to be 96% by iTLC.

### Example 1-F

### (3S,10S,14S)-1-{(1r,4S)-4-[({[4-(3-[bis(2-{[3-hydroxy-1-(2-hydroxyethyl)-2-oxo-1,2-dihydropyridine-4-carbonyl]amino}ethyl)amino]-2-{[bis(2-{[3-hydroxy-1-(2-hydroxyethyl)-2-oxo-1,2-dihydropyridine-4-carbonyl]amino}ethyl)amino]methyl}propyl)phenyl]carbamothioyl}amino)methyl]cyclohexyl}-3-[(naphthalen-2-yl)methyl]-1,4,12-trioxo-2,5,11,13-tetraazahexadecane-10,14,16-tricarboxylic acid

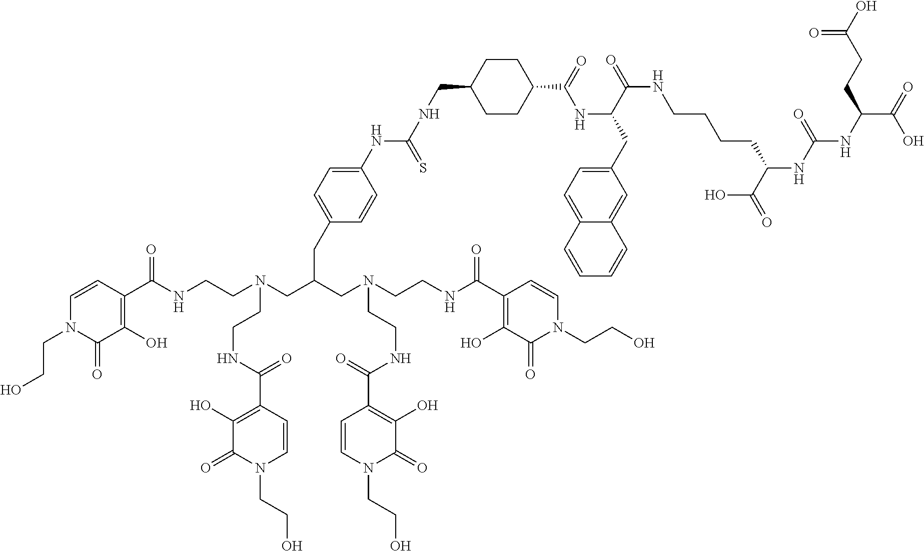 [SMILES: O=C(O)CC[C@H](NC(=O)N[C@@H](CCCCNC(=O)[C@H](Cc1ccc2ccccc2c1)NC(=O)[C@H]1CC[C@H](CNC(=S)Nc2ccc(CC(CN(CCNC(=O)c3ccn(CCO)c(=O)c3O)CCNC(=O)c3ccn(CCO)c(=O)c3O)CN(CCNC(=O)c3ccn(CCO)c(=O)c3O)CCNC(=O)c3ccn(CCO)c(=O)c3O)cc2)CC1)C(=O)O)C(=O)O]

N,N′,N″,N′″-({2-[(4-isothiocyanatophenyl)methyl]propane-1,3-diyl}bis[nitrilodi(ethane-2,1-diyl)])tetrakis[3-hydroxy-1-(2-hydroxyethyl)-2-oxo-1,2-dihydropyridine-4-carboxamide] (9.0 mg, 8.0 μmol) and (3S,10S,14S)-1-[(1r,4S)-4-(aminomethyl)cyclohexyl]-3-[(naphthalen-2-yl)methyl]-1,4,12-trioxo-2,5,11,13-tetraazahexadecane-10,14,16-tricarboxylic acid (3.0 mg, 4.6 μmol) are dissolved in borate buffer (1 mL) and the mixture heated at 50° C. Product is purified using preparative HPLC (RP-HPLC using Äkta pure system, Column: Phenomenex Luna 5 μm C18(2) 100 Å, 250×21.2 mm, Mobile phase: Water/0.1% TFA; ACN Gradient: 20-40% B over 40 min Flow: 10 mL/min Detection: UV 280/335 nm, tR=26 min) affording 3.0 mg (37% yield) of the target compound.

LC-MS (Method K, gradient: 10-50% B over 3 min): Rₜ=1.88 min; MS (ESIpos): m/z=1773.7 [M+H]⁺

### Example 1-F-²²⁷Th

### (3S,10S,14S)-1-{(1r,4S)-4-[({[4-(3-[bis(2-{[3-(hydroxy-kappaO)-1-(2-hydroxyethyl)-2-(oxo-kappaO)-1,2-dihydropyridine-4-carbonyl]amino}ethyl)amino]-2-{[bis(2-{[3-(hydroxy-kappaO)-1-(2-hydroxyethyl)-2-(oxo-kappaO)-1,2-dihydropyridine-4-carbonyl]amino}ethyl)amino]methyl}propyl)phenyl]carbamothioyl}amino)methyl]cyclohexyl}-3-[(naphthalen-2-yl)methyl]-1,4,12-trioxo-2,5,11,13-tetraazahexadecane-10,14,16-tricarboxylato(4−)(²²⁷Th)Thorium

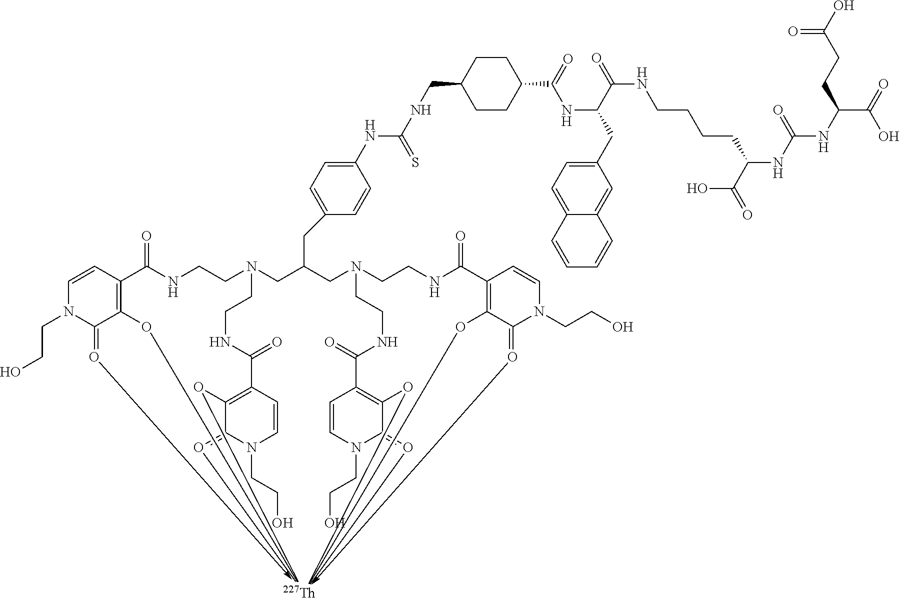

(3S,10S,14S)-1-{(1 r,4S)-4-[({[4-(3-[bis(2-{[3-hydroxy-1-(2-hydroxyethyl)-2-oxo-1,2-dihydropyridine-4-carbonyl]amino}ethyl)amino]-2-{[bis(2-{[3-hydroxy-1-(2-hydroxyethyl)-2-oxo-1,2-dihydropyridine-4-carbonyl]amino}ethyl)amino]methyl}propyl)phenyl]carbamothioyl}amino)methyl]cyclohexyl}-3-[(naphthalen-2-yl)methyl]-1,4,12-trioxo-2,5,11,13-tetraazahexadecane-10,14,16-tricarboxylic acid (6.9 μg) dissolved in 230 μL 30 mM citrate buffer (pH 5.5) containing 0.5 mg/mL pABA was mixed with thorium-227 in 0.5 M HCl (2 μL) at 0.3 MBq/nmol specific activity and RAC of 3.7 MBq/mL and the mixture incubated for 60 min. The labelling efficiency was determined to be 97% by iTLC.

### Example 1-M

### (3S,10S,14S)-1-[(1r,4S)-4-(acetamidomethyl)cyclohexyl]-3-[(naphthalen-2-yl)methyl]-1,4,12-trioxo-2,5,11,13-tetraazahexadecane-10,14,16-tricarboxylic acid

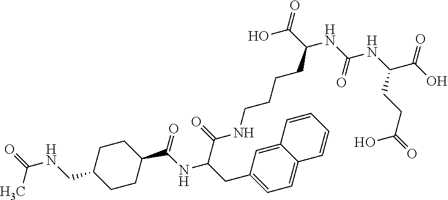 [SMILES: CC(=O)NC[C@H]1CC[C@H](C(=O)NC(Cc2ccc3ccccc3c2)C(=O)NCCCC[C@H](NC(=O)N[C@@H](CCC(=O)O)C(=O)O)C(=O)O)CC1]

tri-tert-butyl (3S,10S,14S)-1-[(1r,4S)-4-(acetamidomethyl)cyclohexyl]-3-[(naphthalen-2-yl)methyl]-1,4,12-trioxo-2,5,11,13-tetraazahexadecane-10,14,16-tricarboxylate (20.0 mg, 98% purity, 22.6 μmol) was solubilised in DCM (1.0 ml), TFA (1.0 ml, 13 mmol) was added and the mixture was stirred under argon at rt. The mixture was evaporated and purified by preparative HPLC (C18, acetonitrile/water with 0.1% formic acid) to give 8.00 mg (96% purity, 49% yield) of the target compound.

LC-MS (Method 1): Rₜ=0.86 min; MS (ESIpos): m/z=699 [M+H]⁺

¹H-NMR (400 MHz, DMSO-d6) δ[ppm]: 0.755 (0.43), 0.786 (0.72), 0.818 (0.58), 1.009 (0.43), 1.178 (0.65), 1.231 (1.52), 1.300 (0.87), 1.319 (0.87), 1.352 (0.43), 1.461 (0.87), 1.482 (0.58), 1.577 (0.80), 1.612 (0.87), 1.772 (13.39), 1.907 (0.80), 2.062 (0.58), 2.217 (0.80), 2.235 (1.52), 2.254 (0.65), 2.332 (2.97), 2.336 (1.30), 2.518 (16.00), 2.523 (10.79), 2.673 (3.04), 2.678 (1.30), 2.810 (1.01), 2.826 (1.59), 2.841 (0.94), 2.887 (0.51), 2.910 (0.51), 2.921 (0.58), 2.945 (0.65), 2.984 (0.51), 2.999 (0.58), 3.017 (0.51), 3.034 (0.58), 3.074 (0.58), 3.088 (0.65), 3.109 (0.51), 3.122 (0.43), 3.998 (0.58), 4.011 (0.58), 4.070 (0.58), 4.086 (0.58), 4.513 (0.58), 4.526 (0.58), 6.280 (0.65), 6.298 (1.09), 6.317 (0.65), 7.381 (1.01), 7.385 (1.01), 7.402 (1.09), 7.406 (1.16), 7.419 (0.43), 7.433 (1.09), 7.437 (0.94), 7.447 (1.16), 7.453 (1.52), 7.456 (1.30), 7.467 (0.94), 7.471 (1.09), 7.484 (0.43), 7.681 (1.88), 7.726 (0.51), 7.741 (0.94), 7.755 (0.43), 7.772 (1.09), 7.783 (1.74), 7.789 (1.09), 7.804 (1.45), 7.839 (1.01), 7.857 (0.94), 7.943 (1.23), 7.955 (1.23), 7.965 (1.09).

Intermediate 10

### di-tert-butyl N-{[(2S)-6-{[(benzyloxy)carbonyl]amino}-1-tert-butoxy-1-oxohexan-2-yl]carbamoyl}-D-glutamate

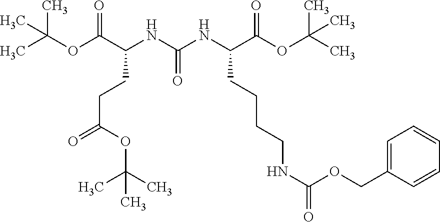 [SMILES: CC(C)(C)OC(=O)CC[C@@H](NC(=O)N[C@@H](CCCCNC(=O)OCc1ccccc1)C(=O)OC(C)(C)C)C(=O)OC(C)(C)C]

di-tert-butyl D-glutamate-hydrogen chloride (1/1) (4.97 g, 16.8 mmol) and 4-nitrophenyl carbonochloridate (3.57 g, 17.7 mmol) were solubilised in DCM (51 ml), cooled to 0° C. under argon, and N,N-diisopropylethylamine (6.7 ml, 39 mmol) was added dropwise. The mixture was stirred for 5 min at 0° C. and 30 min at rt. tert-butyl N6-[(benzyloxy)carbonyl]-L-lysinate-hydrogen chloride (1/1) (7.22 g, 19.4 mmol) was added and N,N-diisopropylethylamine (6.7 ml, 39 mmol) was added dropwise to the mixture. It was stirred 1 h at rt. The mixture was washed 3 times with sat. sodium hydrogen carbonate, once with sodium hydroxide (1.0 M) and once with brine. The organic layer was dried and concentrated under reduced pressure to give 13.3 g (78% purity, 99% yield) of the target compound, which was used without further purification.

LC-MS (Method 1): Rₜ=1.47 min; MS (ESIpos): m/z=623 [M+H]⁺

¹H-NMR (400 MHz, DMSO-d6) δ[ppm]: 1.409 (0.86), 1.419 (0.83), 1.447 (16.00), 1.737 (0.56), 1.754 (0.54), 2.518 (0.43), 2.983 (0.68), 2.997 (0.69), 3.836 (0.75), 4.995 (2.79), 7.270 (0.50), 7.307 (0.75), 7.321 (0.90), 7.324 (0.93), 7.327 (0.91), 7.337 (2.40), 7.348 (2.00), 7.361 (0.84), 7.365 (1.00), 7.368 (0.64), 8.345 (0.80).

Intermediate 11

### di-tert-butyl N-{[(2S)-6-amino-1-tert-butoxy-1-oxohexan-2-yl]carbamoyl}-D-glutamate

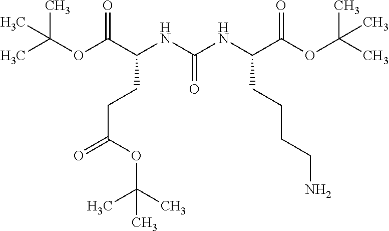 [SMILES: CC(C)(C)OC(=O)CC[C@@H](NC(=O)N[C@@H](CCCCN)C(=O)OC(C)(C)C)C(=O)OC(C)(C)C]

di-tert-butyl N-{[(2S)-6-{[(benzyloxy)carbonyl]amino}-1-tert-butoxy-1-oxohexan-2-yl]carbamoyl}-D-glutamate (2.37 g, 70% purity, 2.67 mmol) was solubilised in MeOH (40 ml), palladium on carbon (60.0 mg, 10% purity) was added and the mixture was purged with hydrogen. The mixture was stirred for 4.5 h at rt under hydrogen atmosphere. The mixture was filtered over Celite, washed with MeOH and concentrated under reduced pressure to give 1.80 g (70% purity, 97% yield) of the target compound, which was used without further purification.

LC-MS (Method 1): Rₜ=0.98 min; MS (ESIpos): m/z=489 [M+H]⁺

¹H-NMR (400 MHz, DMSO-d6) δ[ppm]: 1.389 (16.00), 1.397 (9.42), 2.478 (0.42), 2.728 (1.77), 2.888 (2.10), 6.319 (0.43).

Intermediate 12

### tri-tert-butyl (5S,12S,16R)-1-(9H-fluoren-9-yl)-5-[(naphthalen-2-yl)methyl]-3,6,14-trioxo-2-oxa-4,7,13,15-tetraazaoctadecane-12,16,18-tricarboxylate

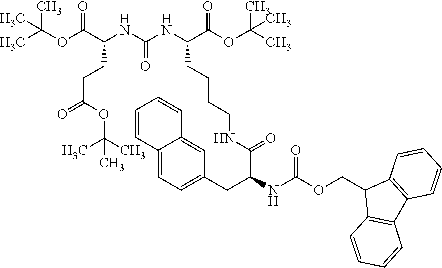 [SMILES: CC(C)(C)OC(=O)CC[C@@H](NC(=O)N[C@@H](CCCCNC(=O)[C@H](Cc1ccc2ccccc2c1)NC(=O)OCC1c2ccccc2-c2ccccc21)C(=O)OC(C)(C)C)C(=O)OC(C)(C)C]

di-tert-butyl N-{[(2S)-6-amino-1-tert-butoxy-1-oxohexan-2-yl]carbamoyl}-D-glutamate (4.00 g, 85% purity, 6.97 mmol) and (2S)-2-({[(9H-fluoren-9-yl)methoxy]carbonyl}amino)-3-(naphthalen-2-yl)propanoic acid (4.58 g, 10.5 mmol; CAS-RN:[112883-43-9]) were solubilised in DMF (54 ml), 4-methylmorpholine (2.3 ml, 21 mmol, CAS-RN: 109-02-4) and HATU (3.98 g, 10.5 mmol) were added and the mixture was stirred under argon overnight at rt. The mixture was evaporated, diluted with water and extracted 3 times with DCM. The combined organic layers were washed with brine, dried and evaporated. The residue was purified by flash chromatography (SiO2, hexane/EtOAc gradient 0%-25%) to give 6.07 g (96% yield) of the target compound.

LC-MS (Method 1): Rₜ=1.65 min; MS (ESIpos): m/z=908 [M+H]⁺

¹H-NMR (400 MHz, DMSO-d6) δ[ppm]: 1.317 (0.43), 1.368 (9.34), 1.382 (7.41), 1.389 (16.00), 2.518 (1.06), 2.522 (0.73), 3.096 (0.40), 4.070 (1.47), 4.089 (1.18), 4.097 (1.55), 4.106 (0.87), 6.310 (0.42), 7.121 (0.44), 7.138 (0.44), 7.155 (0.44), 7.213 (0.60), 7.232 (0.68), 7.250 (0.42), 7.336 (0.76), 7.354 (1.06), 7.372 (0.76), 7.381 (0.57), 7.423 (0.60), 7.434 (0.92), 7.440 (1.20), 7.449 (1.40), 7.459 (1.25), 7.475 (0.62), 7.524 (0.71), 7.545 (0.63), 7.564 (0.66), 7.583 (0.65), 7.591 (0.65), 7.609 (0.57), 7.667 (0.55), 7.688 (0.84), 7.750 (0.85), 7.760 (0.79), 7.780 (0.83), 7.800 (1.13), 7.821 (1.86), 7.837 (1.77), 7.853 (1.11), 7.861 (0.91).

Intermediate 13

### di-tert-butyl (2R)-2-({[(2S)-6-{[(2S)-2-amino-3-(naphthalen-2-yl)propanoyl]amino}-1-tert-butoxy-1-oxohexan-2-yl]carbamoyl}amino)pentanedioate

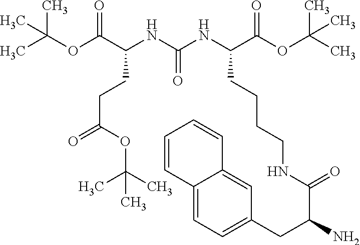 [SMILES: CC(C)(C)OC(=O)CC[C@@H](NC(=O)N[C@@H](CCCCNC(=O)[C@@H](N)Cc1ccc2ccccc2c1)C(=O)OC(C)(C)C)C(=O)OC(C)(C)C]

tri-tert-butyl (5S,12S,16R)-1-(9H-fluoren-9-yl)-5-[(naphthalen-2-yl)methyl]-3,6,14-trioxo-2-oxa-4,7,13,15-tetraazaoctadecane-12,16,18-tricarboxylate (940 mg, 95% purity, 984 μmol) was solubilised in DMF (26 ml), piperidine (1.9 ml, 20 mmol) was added and the mixture was stirred under argon over the weekend at rt. The mixture was evaporated and purified by preparative HPLC (C18, acetonitrile/water with 0.2% ammonia) to give 305 mg (98% purity, 44% yield) of the target compound.

LC-MS (Method 1): Rₜ=1.21 min; MS (ESIpos): m/z=686 [M+H]⁺

Intermediate 14

### tri-tert-butyl (3S,10S,14R)-1-{(1r,4S)-4-[({[(9H-fluoren-9-yl)methoxy]carbonyl}amino)methyl]cyclohexyl}-3-[(naphthalen-2-yl)methyl]-1,4,12-trioxo-2,5,11,13-tetraazahexadecane-10,14,16-tricarboxylate

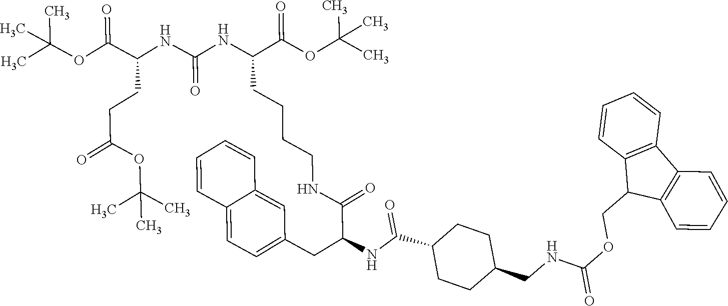 [SMILES: CC(C)(C)OC(=O)CC[C@@H](NC(=O)N[C@@H](CCCCNC(=O)[C@H](Cc1ccc2ccccc2c1)NC(=O)[C@H]1CC[C@H](CNC(=O)OCC2c3ccccc3-c3ccccc32)CC1)C(=O)OC(C)(C)C)C(=O)OC(C)(C)C]

di-tert-butyl (2R)-2-({[(2S)-6-{[(2S)-2-amino-3-(naphthalen-2-yl)propanoyl]amino}-1-tert-butoxy-1-oxohexan-2-yl]carbamoyl}amino)pentanedioate (305 mg, 445 μmol) and (1r,4r)-4-[({[(9H-fluoren-9-yl)methoxy]carbonyl}amino)methyl]cyclohexane-1-carboxylic acid (253 mg, 668 μmol) were solubilised in DMF (3.4 ml), 4-methylmorpholine (150 μl, 1.3 mmol, CAS-RN: 109-02-4) and HATU (254 mg, 668 μmol) were added and the mixture was stirred under argon overnight at rt. The mixture was purified by preparative HPLC (C18, acetonitrile/water with 0.1% formic acid) to give 160 mg (96% purity, 33% yield) of the target compound.

LC-MS (Method 1): Rₜ=1.65 min; MS (ESIpos): m/z=1047 [M+H]⁺

¹H-NMR (400 MHz, DMSO-d6) δ[ppm]: 1.214 (0.59), 1.230 (0.78), 1.373 (16.00), 1.379 (4.03), 1.387 (15.09), 1.390 (15.72), 1.643 (0.40), 2.297 (0.85), 2.522 (0.43), 2.781 (0.46), 4.280 (0.88), 4.297 (0.62), 6.312 (0.46), 6.333 (0.42), 7.245 (0.48), 7.289 (0.41), 7.306 (0.86), 7.324 (0.55), 7.382 (0.77), 7.400 (0.95), 7.418 (0.53), 7.448 (0.61), 7.668 (0.87), 7.686 (1.10), 7.788 (0.65), 7.808 (0.53), 7.873 (0.95), 7.892 (0.84), 7.944 (0.51).

Intermediate 15

### tri-tert-butyl (3S,10S,14R)-1-[(1r,4S)-4-(aminomethyl)cyclohexyl]-3-[(naphthalen-2-yl)methyl]-1,4,12-trioxo-2,5,11,13-tetraazahexadecane-10,14,16-tricarboxylate

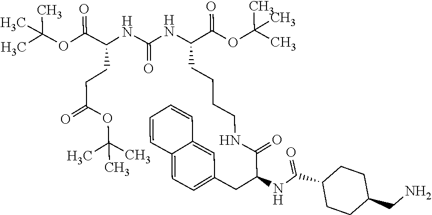 [SMILES: CC(C)(C)OC(=O)CC[C@@H](NC(=O)N[C@@H](CCCCNC(=O)[C@H](Cc1ccc2ccccc2c1)NC(=O)[C@H]1CC[C@H](CN)CC1)C(=O)OC(C)(C)C)C(=O)OC(C)(C)C]

tri-tert-butyl (3S,10S,14R)-1-{(1r,4S)-4-[({[(9H-fluoren-9-yl)methoxy]carbonyl}amino)methyl]cyclohexyl}-3-[(naphthalen-2-yl)methyl]-1,4,12-trioxo-2,5,11,13-tetraazahexadecane-10,14,16-tricarboxylate (160 mg, 96% purity, 147 μmol) was solubilised in DMF (3.9 ml), piperidine (290 μl, 2.9 mmol) was added and the mixture was stirred under argon at rt. The mixture was evaporated and purified by preparative HPLC (C18, acetonitrile/water with 0.1% formic acid) to give 95.0 mg (100% purity, 79% yield) of the target compound.

LC-MS (Method 1): Rₜ=1.18 min; MS (ESIpos): m/z=825 [M+H]⁺

¹H-NMR (400 MHz, DMSO-d6) δ[ppm]: 1.195 (0.43), 1.232 (0.47), 1.377 (16.00), 1.388 (13.40), 1.394 (14.23), 1.677 (0.40), 2.332 (0.64), 2.518 (3.55), 2.523 (2.55), 2.673 (0.66), 6.332 (0.42), 7.452 (0.74), 7.678 (0.66), 7.784 (0.71), 7.804 (0.53), 7.960 (0.42), 7.981 (0.64), 8.411 (1.22).

### Example 1-A-D

### (3S,10S,14R)-1-[(1r,4S)-4-(aminomethyl)cyclohexyl]-3-[(naphthalen-2-yl)methyl]-1,4,12-trioxo-2,5,11,13-tetraazahexadecane-10,14,16-tricarboxylic acid

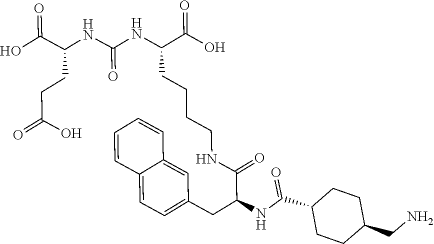 [SMILES: NC[C@H]1CC[C@H](C(=O)N[C@@H](Cc2ccc3ccccc3c2)C(=O)NCCCC[C@H](NC(=O)N[C@H](CCC(=O)O)C(=O)O)C(=O)O)CC1]

tri-tert-butyl (3S,10S,14R)-1-[(1r,4S)-4-(aminomethyl)cyclohexyl]-3-[(naphthalen-2-yl)methyl]-1,4,12-trioxo-2,5,11,13-tetraazahexadecane-10,14,16-tricarboxylate (21.9 mg, 98% purity, 26.0 μmol) was solubilised in DCM (1.6 ml), TFA (1.6 ml, 21 mmol) was added and the mixture was stirred under argon at rt. The mixture was evaporated and purified by preparative HPLC (C18, acetonitrile/water with 0.1% formic acid) to give 6.00 mg (95% purity, 33% yield) of the target compound.

LC-MS (Method 1): Rₜ=0.73 min; MS (ESIpos): m/z=657 [M+H]⁺

¹H-NMR (400 MHz, DMSO-d6) δ[ppm]: 0.886 (1.25), 0.912 (1.33), 1.067 (0.86), 1.094 (0.86), 1.145 (0.62), 1.251 (2.42), 1.314 (2.50), 1.330 (2.58), 1.522 (2.73), 1.650 (1.95), 1.829 (0.94), 1.903 (0.70), 2.078 (1.25), 2.116 (1.17), 2.137 (1.01), 2.151 (1.17), 2.287 (0.78), 2.332 (3.43), 2.336 (1.64), 2.451 (2.58), 2.518 (14.91), 2.523 (10.69), 2.562 (16.00), 2.579 (2.50), 2.594 (2.81), 2.678 (1.48), 2.916 (1.09), 2.949 (1.72), 2.974 (1.64), 3.023 (2.34), 3.090 (2.34), 3.102 (2.50), 3.124 (2.34), 3.137 (2.19), 3.350 (5.70), 3.906 (1.64), 4.501 (1.25), 4.515 (1.25), 7.389 (2.34), 7.413 (2.97), 7.430 (2.73), 7.433 (2.65), 7.442 (2.89), 7.448 (5.07), 7.453 (2.97), 7.462 (2.50), 7.466 (2.58), 7.479 (1.01), 7.690 (4.60), 7.778 (6.56), 7.800 (5.78), 7.832 (2.65), 7.836 (2.65), 7.854 (2.50), 8.290 (0.86).

### Example 1-B-D

### (3S,10S,14R)-3-[(naphthalen-2-yl)methyl]-1,4,12-trioxo-1-[(1r,4S)-4-({2-[4,7,10-tris(carboxymethyl)-1,4,7,10-tetraazacyclododecan-1-yl]acetamido}methyl)cyclohexyl]-2,5,11,13-tetraazahexadecane-10,14,16-tricarboxylic acid

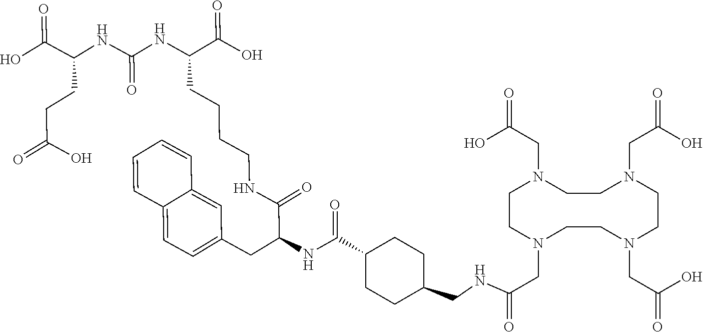 [SMILES: O=C(O)CC[C@@H](NC(=O)N[C@@H](CCCCNC(=O)[C@H](Cc1ccc2ccccc2c1)NC(=O)[C@H]1CC[C@H](CNC(=O)CN2CCN(CC(=O)O)CCN(CC(=O)O)CCN(CC(=O)O)CC2)CC1)C(=O)O)C(=O)O]

tri-tert-butyl (3S,10S,14R)-3-[(naphthalen-2-yl)methyl]-1,4,12-trioxo-1-[(1r,4S)-4-({2-[4,7,10-tris(2-tert-butoxy-2-oxoethyl)-1,4,7,10-tetraazacyclododecan-1-yl]acetamido}methyl)cyclohexyl]-2,5,11,13-tetraazahexadecane-10,14,16-tricarboxylate (30.0 mg, 21.8 μmol) was solubilised in DCM (280 μl), TFA (840 μl, 11 mmol) was added and the mixture was stirred under argon overnight at rt. The mixture was evaporated and purified by preparative HPLC (C18, acetonitrile/water with 0.1% formic acid) to give 6.00 mg (95% purity, 25% yield) of the target compound.

¹H-NMR (400 MHz, DMSO-d6) δ[ppm]: 0.820 (0.80), 0.851 (0.54), 1.091 (0.47), 1.119 (0.47), 1.232 (2.01), 1.310 (1.27), 1.325 (1.41), 1.468 (1.14), 1.489 (0.87), 1.584 (0.94), 1.618 (1.00), 1.665 (0.94), 1.707 (0.87), 1.729 (0.67), 1.745 (0.54), 1.857 (0.54), 1.907 (0.74), 2.022 (0.47), 2.053 (0.67), 2.074 (0.67), 2.190 (0.87), 2.206 (1.34), 2.227 (1.27), 2.244 (0.74), 2.318 (1.47), 2.322 (3.08), 2.326 (4.15), 2.331 (3.08), 2.336 (1.47), 2.518 (16.00), 2.522 (10.31), 2.636 (1.87), 2.659 (2.21), 2.664 (3.62), 2.668 (4.55), 2.673 (3.35), 2.678 (1.67), 2.899 (2.14), 2.957 (4.15), 2.996 (4.08), 3.081 (2.54), 3.115 (1.21), 3.429 (6.03), 3.991 (0.47), 4.011 (0.80), 4.023 (0.80), 4.043 (0.40), 4.066 (0.40), 4.086 (0.80), 4.101 (0.80), 4.465 (0.40), 4.488 (0.67), 4.501 (0.67), 6.350 (0.94), 6.364 (1.27), 6.383 (0.94), 7.384 (1.27), 7.388 (1.27), 7.408 (1.41), 7.418 (0.60), 7.431 (1.34), 7.435 (1.27), 7.452 (2.08), 7.466 (1.21), 7.469 (1.27), 7.483 (0.54), 7.689 (2.34), 7.785 (3.08), 7.805 (2.61), 7.838 (1.34), 7.856 (1.21), 7.916 (0.74), 8.068 (1.21).

### Example 1-B-D-²²⁷Th

### [²²⁷Th]Thorium-(3S,10S,14R)-3-[(naphthalen-2-yl)methyl]-1,4,12-trioxo-1-{(1r,4S)-4-[(2-{4,7,10-tris[(carboxy-kappaO)methyl]-1,4,7,10-tetraazacyclododecan-1-yl-kappa⁴N¹,N⁴,N⁷,N¹⁰}acetamido)methyl]cyclohexyl}-2,5,11,13-tetraazahexadecane-10,14,16-tricarboxylate

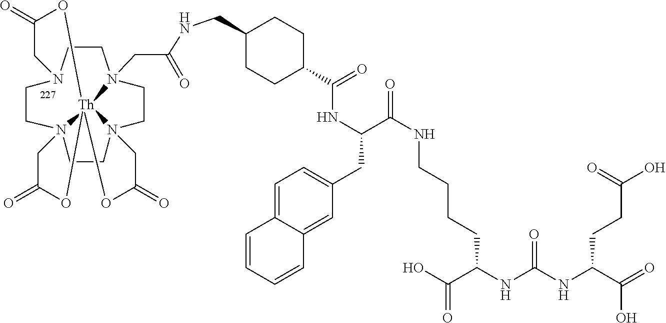

(3S,10S,14R)-3-[(naphthalen-2-yl)methyl]-1,4,12-trioxo-1-[(1r,4S)-4-({2-[4,7,10-tris(carboxymethyl)-1,4,7,10-tetraazacyclododecan-1-yl]acetamido}methyl)cyclohexyl]-2,5,11,13-tetraazahexadecane-10,14,16-tricarboxylic acid was dissolved in 400 mM sodium acetate buffer (pH 5) containing 0.5 mg/mL pABA. Thorium-227 in 400 mM sodium acetate buffer (pH 5) plus 20% EtOH was added giving a specific activity of 0.375 MBq/nmol and a RAC of 7.1 MBq/mL. The mixture was incubated at 95° C. for 60 min. The labelling efficiency was determined to be 96.7% by iTLC.

Intermediate 6-Br

### tri-tert-butyl (3S,10S,14S)-1-[(1r,4S)-4-(aminomethyl)cyclohexyl]-3-[(1-bromonaphthalen-2-yl)methyl]-1,4,12-trioxo-2,5,11,13-tetraazahexadecane-10,14,16-tricarboxylate

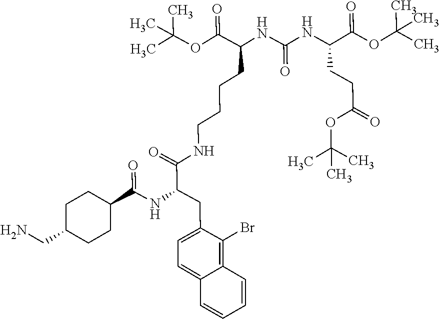 [SMILES: CC(C)(C)OC(=O)CC[C@H](NC(=O)N[C@@H](CCCCNC(=O)[C@H](Cc1ccc2ccccc2c1Br)NC(=O)[C@H]1CC[C@H](CN)CC1)C(=O)OC(C)(C)C)C(=O)OC(C)(C)C]

N-Bromo-succinimide (7.60 mg, 42.7 μmol; CAS-RN:[128-08-5]) was added portionwise at r.t. to a solution of tri-tert-butyl (3S,10S,14S)-1-[(1r,4S)-4-(aminomethyl)cyclohexyl]-3-[(naphthalen-2-yl)methyl]-1,4,12-trioxo-2,5,11,13-tetraazahexadecane-10,14,16-tricarboxylate (32.0 mg, 38.8 μmol) in acetonitrile (1.9 mL). After 3.5 h further N-bromo-succinimide (15.2 mg, 85.4 μmol) was added and the mixture was stirred for further 3 d in the dark at r.t. Then the mixture was filtered and the filtrate was concentrated under reduced pressure. The crude product was purified by preparative HPLC (C18, acetonitrile/0.1% formic acid gradient) to give 13.0 mg of the final product (37% yield).

¹H-NMR (400 MHz, DMSO-d6) δ[ppm]: 1.232 (0.67), 1.353 (0.50), 1.382 (13.48), 1.389 (16.00), 1.907 (0.90), 2.327 (3.24), 2.331 (2.35), 2.336 (1.06), 2.518 (13.82), 2.523 (8.45), 2.562 (2.85), 2.669 (3.30), 2.673 (2.41), 2.678 (1.06), 7.868 (0.45).

Intermediate 7-Br

### tri-tert-butyl (3S,10S,14S)-3-[(1-bromonaphthalen-2-yl)methyl]-1,4,12-trioxo-1-[(1r,4S)-4-({2-[4,7,10-tris(2-tert-butoxy-2-oxoethyl)-1,4,7,10-tetraazacyclododecan-1-yl]acetamido}methyl)cyclohexyl]-2,5,11,13-tetraazahexadecane-10,14,16-tricarboxylate

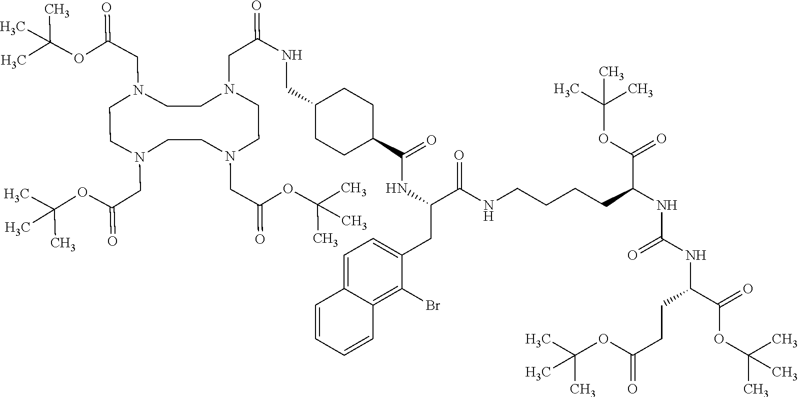 [SMILES: CC(C)(C)OC(=O)CC[C@H](NC(=O)N[C@@H](CCCCNC(=O)[C@H](Cc1ccc2ccccc2c1Br)NC(=O)[C@H]1CC[C@H](CNC(=O)CN2CCN(CC(=O)OC(C)(C)C)CCN(CC(=O)OC(C)(C)C)CCN(CC(=O)OC(C)(C)C)CC2)CC1)C(=O)OC(C)(C)C)C(=O)OC(C)(C)C]

N,N-Diisopropylethylamine (6.9 μl, 40 μmol; CAS-RN:[7087-68-5]) was added at r.t. to a solution of [4,7,10-tris(2-tert-butoxy-2-oxoethyl)-1,4,7,10-tetraazacyclododecan-1-yl]acetic acid (22.8 mg, 39.9 μmol) and [(1H-benzotriazol-1-yl)oxy](dimethylamino)-N,N-dimethylmethaniminium hexafluoridophosphate(1−) (14.9 mg, 39.4 μmol; CAS-RN:[94790-37-1]) in DMF (270 μl, 3.6 mmol; CAS-RN:[68-12-2]). After 15 min tri-tert-butyl (3S,10S,14S)-1-[(1r,4S)-4-(aminomethyl)cyclohexyl]-3-[(1-bromonaphthalen-2-yl)methyl]-1,4,12-trioxo-2,5,11,13-tetraazahexadecane-10,14,16-tricarboxylate (9.00 mg, 9.97 μmol) was added and the mixture stirred for 2 h at r.t. After that the mixture was purified by preparative HPLC (C18, acetonitrile/0.1% formic acid gradient) to give 8.6 mg of the title compound (59% yield).

LC-MS (Method 1): Rₜ=1.36 min; MS (ESIpos): m/z=1459 [M+H]⁺

Intermediate 7-3H

### tri-tert-butyl (3S,10S,14S)-3-{[(1-³H)naphthalen-2-yl]methyl}-1,4,12-trioxo-1-[(1r,4S)-4-({2-[4,7,10-tris(2-tert-butoxy-2-oxoethyl)-1,4,7,10-tetraazacyclododecan-1-yl]acetamido}methyl)cyclohexyl]-2,5,11,13-tetraazahexadecane-10,14,16-tricarboxylate

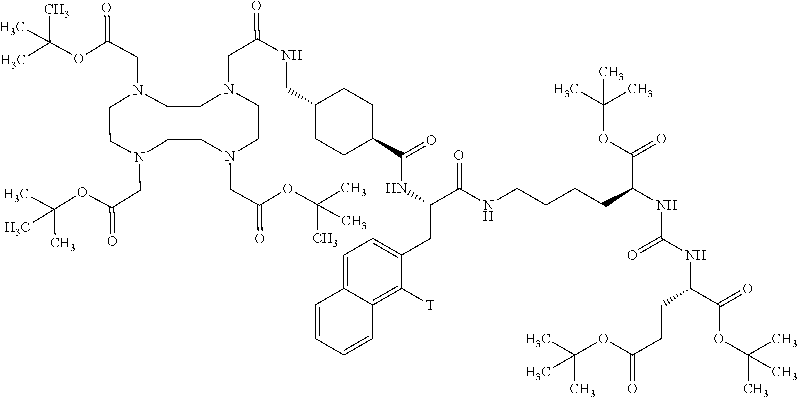

A mixture of 3.3 mg tri-tert-butyl (3S,10S,14S)-3-[(1-bromonaphthalen-2-yl)methyl]-1,4,12-trioxo-1-[(1r,4S)-4-({2-[4,7,10-tris(2-tert-butoxy-2-oxoethyl)-1,4,7,10-tetraazacyclododecan-1-yl]acetamido}methyl)cyclohexyl]-2,5,11,13-tetraazahexadecane-10,14,16-tricarboxylate (2.27 μmol) in 2.00 mL THF, 10 μL triethylamine and 11.8 mg 10% palladium on charcoal were stirred at a tritium pressure of 270 mbar for 2 h at room temperature. After freezing of the reaction mixture with liquid nitrogen the non-reacted tritium was adsorbed on a waste storage filled with charcoal and platinum oxide. The solvent was evaporated and the dry residue was dissolved in 2.5 mL THF/EtOH (1+1, v+v) and the catalyst was filtered off using a 0.45 μm PTFE syringe filter (Whatman). Labile tritium was removed by dissolving the residue in THF/EtOH (1+1, v+v) followed by evaporation (five times). The crude product solution was purified by RP-chromatography (method O) yielding 919.3 MBq (1.36 mg, 0.98 μmol) of the title compound with a specific activity of 933.7 GBq/mmol.

LC-MS (Method P): Rt=1.33; m/z=1378.9 (M+H)+, 1380.9 (M+H)+, 1382.9 (M+H)+

Radio-HPLC (Method Q): Rₜ=20.9 min

### Example 1-B-3H, 3H-PSMA-617

### (3S,10S,14S)-3-{[(3-³H)naphthalen-2-yl]methyl}-1,4,12-trioxo-1-[(1r,4S)-4-({2-[4,7,10-tris(carboxymethyl)-1,4,7,10-tetraazacyclododecan-1-yl]acetamido}methyl)cyclohexyl]-2,5,11,13-tetraazahexadecane-10,14,16-tricarboxylic acid

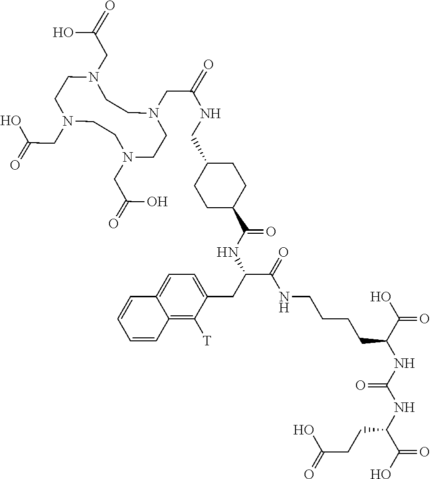

750 MBq tri-tert-butyl (3S,10S,14S)-3-{[(1-³H)naphthalen-2-yl]methyl}-1,4,12-trioxo-1-[(1r,4S)-4-({2-[4,7,10-tris(2-tert-butoxy-2-oxoethyl)-1,4,7,10-tetraazacyclododecan-1-yl]acetamido}methyl)cyclohexyl]-2,5,11,13-tetraazahexadecane-10,14,16-tricarboxylate (1.11 mg, 0.80 μmol) were dissolved in 2.00 mL 4 M HCl in dioxane/water (1.9+0.1, v+v) and stirred for 1.5 h at room temperature. The solvent war removed under reduced pressure and purified by RP-chromatography (method R) yielding 520.7 MBq (0.59 mg, 0.56 μmol) of the title compound with a specific activity of 926.6 GBq/mmol (70%, >99% radiochemical purity).

LC-MS (Method S): Rₜ=1.11 min; m/z=1042.1 (M+H)⁺, 1044.1 (M+H)⁺, 1046.1 (M+H)⁺

Radio-HPLC (Method T): Rₜ=10.0 min;

#2 Linker

Intermediate 9

### tri-tert-butyl (5S,12S,16S)-1-(9H-fluoren-9-yl)-5-[(3-methylphenyl)methyl]-3,6,14-trioxo-2-oxa-4,7,13,15-tetraazaoctadecane-12,16,18-tricarboxylate

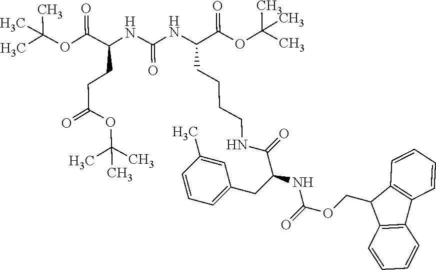 [SMILES: Cc1cccc(C[C@H](NC(=O)OCC2c3ccccc3-c3ccccc32)C(=O)NCCCC[C@H](NC(=O)N[C@@H](CCC(=O)OC(C)(C)C)C(=O)OC(C)(C)C)C(=O)OC(C)(C)C)c1]

di-tert-butyl N-{[(2S)-6-amino-1-tert-butoxy-1-oxohexan-2-yl]carbamoyl}-L-glutamate (300 mg, 70% purity, 431 μmol) and N-{[(9H-fluoren-9-yl)methoxy]carbonyl}-3-methyl-L-phenylalanine (259 mg, 646 μmol) were solubilised in DMF (3.3 ml), 4-methylmorpholine (140 μl, 1.3 mmol, CAS-RN: 109-02-4) and HATU (246 mg, 646 μmol) were added and the mixture was stirred under argon overnight at rt. The mixture was evaporated and purified by preparative HPLC (C18, acetonitrile/water with 0.1% formic acid) to give 310 mg (75% purity, 62% yield) of the target compound.

LC-MS (Method 1): Rₜ=1.65 min; MS (ESIpos): m/z=872 [M+H]⁺

¹H-NMR (400 MHz, DMSO-d6) δ[ppm]: 1.380 (16.00), 1.383 (14.47), 2.235 (1.64), 2.297 (0.41), 2.518 (1.77), 2.522 (1.21), 4.131 (0.58), 4.152 (0.45), 7.081 (0.42), 7.403 (0.41), 7.657 (0.47), 7.868 (0.61), 7.887 (0.57).

Intermediate 10

### di-tert-butyl (2S)-2-({[(2S)-6-{[(2S)-2-amino-3-(3-methylphenyl)propanoyl]amino}-1-tert-butoxy-1-oxohexan-2-yl]carbamoyl}amino)pentanedioate

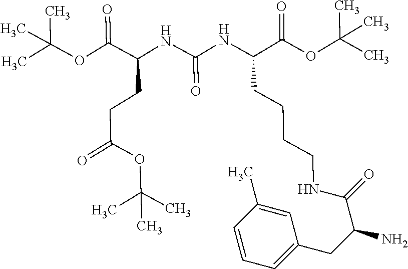 [SMILES: Cc1cccc(C[C@H](N)C(=O)NCCCC[C@H](NC(=O)N[C@@H](CCC(=O)OC(C)(C)C)C(=O)OC(C)(C)C)C(=O)OC(C)(C)C)c1]

tri-tert-butyl (5S,12S,16S)-1-(9H-fluoren-9-yl)-5-[(3-methylphenyl)methyl]-3,6,14-trioxo-2-oxa-4,7,13,15-tetraazaoctadecane-12,16,18-tricarboxylate (310 mg, 92% purity, 327 μmol) was solubilised in DMF (8.6 ml), piperidine (650 μl, 6.5 mmol) was added and the mixture was stirred under argon overnight at rt. The mixture was evaporated and purified by preparative HPLC (C18, acetonitrile/water with 0.1% formic acid) to give 130 mg (98% purity, 60% yield) of the target compound.

LC-MS (Method 1): Rₜ=1.39 min; MS (ESIpos): m/z=650 [M+H]⁺

Intermediate 11

### tri-tert-butyl (3S,10S,14S)-1-{(1r,4S)-4-[({[(9H-fluoren-9-yl)methoxy]carbonyl}amino)methyl]cyclohexyl}-3-[(3-methylphenyl)methyl]-1,4,12-trioxo-2,5,11,13-tetraazahexadecane-10,14,16-tricarboxylate

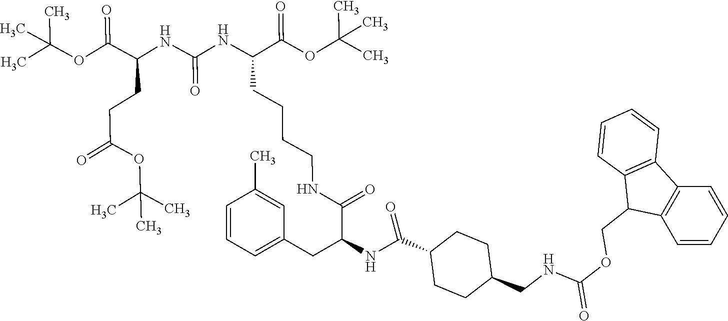 [SMILES: Cc1cccc(C[C@H](NC(=O)[C@H]2CC[C@H](CNC(=O)OCC3c4ccccc4-c4ccccc43)CC2)C(=O)NCCCC[C@H](NC(=O)N[C@@H](CCC(=O)OC(C)(C)C)C(=O)OC(C)(C)C)C(=O)OC(C)(C)C)c1]

di-tert-butyl (2S)-2-({[(2S)-6-{[(2S)-2-amino-3-(3-methylphenyl)propanoyl]amino}-1-tert-butoxy-1-oxohexan-2-yl]carbamoyl}amino)pentanedioate (130 mg, 200 μmol) and (1r,4r)-4-[({[(9H-fluoren-9-yl)methoxy]carbonyl}amino)methyl]cyclohexane-1-carboxylic acid (114 mg, 301 μmol) were solubilised in DMF (1.5 ml), 4-methylmorpholine (66 μl, 600 μmol, CAS-RN: 109-02-4) and HATU (114 mg, 301 μmol) were added and the mixture was stirred under argon overnight at rt. The mixture was evaporated and purified by preparative HPLC (C18, acetonitrile/water with 0.1% formic acid) to give 36.0 mg (18% yield) of the target compound.

LC-MS (Method 1): Rt=1.64 min; MS (ESIpos): m/z=1011 [M+H]⁺

Intermediate 12

### tri-tert-butyl (3S,10S,14S)-1-[(1r,4S)-4-(aminomethyl)cyclohexyl]-3-[(3-methylphenyl)methyl]-1,4,12-trioxo-2,5,11,13-tetraazahexadecane-10,14,16-tricarboxylate

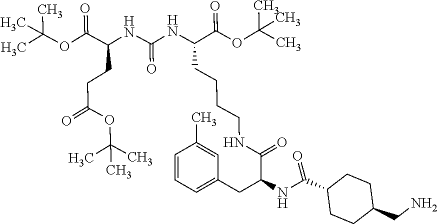 [SMILES: Cc1cccc(C[C@H](NC(=O)[C@H]2CC[C@H](CN)CC2)C(=O)NCCCC[C@H](NC(=O)N[C@@H](CCC(=O)OC(C)(C)C)C(=O)OC(C)(C)C)C(=O)OC(C)(C)C)c1]

tri-tert-butyl (3S,10S,14S)-1-{(1r,4S)-4-[({[(9H-fluoren-9-yl)methoxy]carbonyl}amino)methyl]cyclohexyl}-3-[(3-methylphenyl)methyl]-1,4,12-trioxo-2,5,11,13-tetraazahexadecane-10,14,16-tricarboxylate (36.0 mg, 35.6 μmol) was solubilised in DMF (940 μl), piperidine (70 μl, 710 μmol) was added and the mixture was stirred under argon overnight at rt. The mixture was evaporated and purified by preparative HPLC (C18, acetonitrile/water with 0.1% formic acid) to give 12.0 mg (43% yield) of the target compound.

LC-MS (Method 1): Rt=1.20 min; MS (ESIpos): m/z=789 [M+H]⁺

¹H-NMR (400 MHz, DMSO-d6) δ[ppm]: 1.227 (0.49), 1.381 (14.82), 1.388 (16.00), 2.238 (2.41), 3.509 (1.21), 6.987 (0.47), 7.010 (0.51), 7.113 (0.45).

### Example 2-A

### N⁶—{N-[(1r,4S)-4-(aminomethyl)cyclohexane-1-carbonyl]-3-methyl-L-phenylalanyl}-N²-{[(1S)-1,3-dicarboxypropyl]carbamoyl}-L-lysine

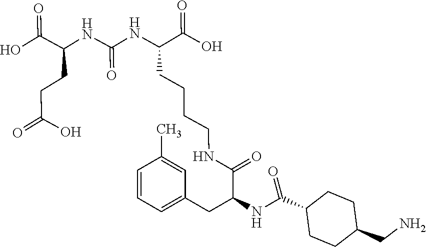 [SMILES: Cc1cccc(C[C@H](NC(=O)[C@H]2CC[C@H](CN)CC2)C(=O)NCCCC[C@H](NC(=O)N[C@@H](CCC(=O)O)C(=O)O)C(=O)O)c1]

tri-tert-butyl (3S,10S,14S)-1-[(1r,4S)-4-(aminomethyl)cyclohexyl]-3-[(3-methylphenyl)methyl]-1,4,12-trioxo-2,5,11,13-tetraazahexadecane-10,14,16-tricarboxylate (12.0 mg, 98% purity, 14.9 μmol) was solubilised in DCM (1.0 ml), TFA (1.0 ml, 13 mmol) was added and the mixture was stirred under argon overnight at rt. The mixture was evaporated and purified by preparative HPLC (C18, acetonitrile/water with 0.1% formic acid) to give 2.00 mg (95% purity, 21% yield) of the target compound.

LC-MS (Method 1): Rₜ=0.66 min; MS (ESIpos): m/z=620 [M+H]⁺

¹H-NMR (400 MHz, DMSO-d6) δ[ppm]: 1.235 (2.71), 2.074 (2.44), 2.242 (11.93), 2.327 (16.00), 2.561 (2.71), 2.609 (2.44), 2.626 (2.44), 2.669 (15.46), 6.985 (2.44), 7.018 (2.71). #3 Linker Intermediate 13 tri-tert-butyl (5S,12S,16S)-1-(9H-fluoren-9-yl)-3,6,14-trioxo-5-(3-phenylpropyl)-2-oxa-4,7,13,15-tetraazaoctadecane-12,16,18-tricarboxylate

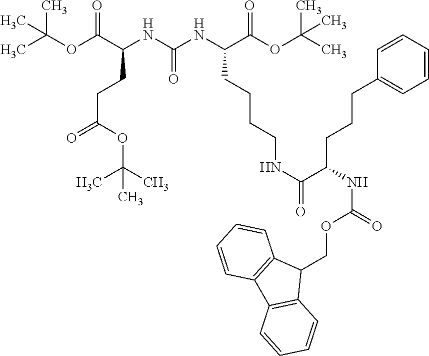 [SMILES: CC(C)(C)OC(=O)CC[C@H](NC(=O)N[C@@H](CCCCNC(=O)[C@H](CCCc1ccccc1)NC(=O)OCC1c2ccccc2-c2ccccc21)C(=O)OC(C)(C)C)C(=O)OC(C)(C)C]

di-tert-butyl N-{[(2S)-6-amino-1-tert-butoxy-1-oxohexan-2-yl]carbamoyl}-L-glutamate (300 mg, 70% purity, 431 μmol) and N-{[(9H-fluoren-9-yl)methoxy]carbonyl}-5-phenyl-L-norvaline (268 mg, 646 μmol) were solubilised in DMF (3.3 ml), 4-methylmorpholine (140 μl, 1.3 mmol, CAS-RN: 109-02-4) and HATU (246 mg, 646 μmol) were added and the mixture was stirred under argon overnight at rt. The mixture was evaporated and purified by preparative HPLC (C18, acetonitrile/water with 0.1% formic acid) to give 300 mg (50% purity, 39% yield) of the target compound.

LC-MS (Method 1): Rₜ=1.67 min; MS (ESIpos): m/z=886 [M+H]⁺

¹H-NMR (400 MHz, DMSO-d6) δ[ppm]: 1.379 (16.00), 2.298 (1.13), 2.518 (0.66), 2.523 (0.46), 7.161 (0.50), 7.163 (0.46), 7.165 (0.43), 7.173 (0.49), 7.179 (0.42), 7.181 (0.42), 7.247 (0.46), 7.264 (0.46), 7.283 (0.43), 7.403 (0.44), 7.874 (0.48), 7.893 (0.56).

Intermediate 14

### di-tert-butyl (2S)-2-({[(2S)-6-{[(2S)-2-amino-5-phenylpentanoyl]amino}-1-tert-butoxy-1-oxohexan-2-yl]carbamoyl}amino)pentanedioate

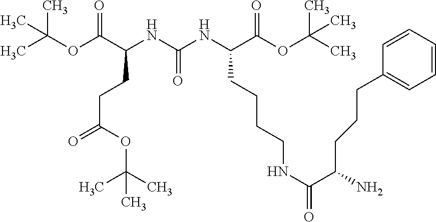 [SMILES: CC(C)(C)OC(=O)CC[C@H](NC(=O)N[C@@H](CCCCNC(=O)[C@@H](N)CCCc1ccccc1)C(=O)OC(C)(C)C)C(=O)OC(C)(C)C]

tri-tert-butyl (5S,12S,16S)-1-(9H-fluoren-9-yl)-3,6,14-trioxo-5-(3-phenylpropyl)-2-oxa-4,7,13,15-tetraazaoctadecane-12,16,18-tricarboxylate (300 mg, 80% purity, 271 μmol) was solubilised in DMF (7.1 ml), piperidine (540 μl, 5.4 mmol) was added and the mixture was stirred under argon overnight at rt. The mixture was evaporated and purified by preparative HPLC (C18, acetonitrile/water with 0.1% formic acid) to give 199 mg (90% purity, 100% yield) of the target compound.

LC-MS (Method 1): Rt=1.15 min; MS (ESIpos): m/z=664 [M+H]⁺

Intermediate 15

### tri-tert-butyl (3S,10S,14S)-1-{(1r,4S)-4-[({[(9H-fluoren-9-yl)methoxy]carbonyl}amino)methyl]cyclohexyl}-1,4,12-trioxo-3-(3-phenylpropyl)-2,5,11,13-tetraazahexadecane-10,14,16-tricarboxylate

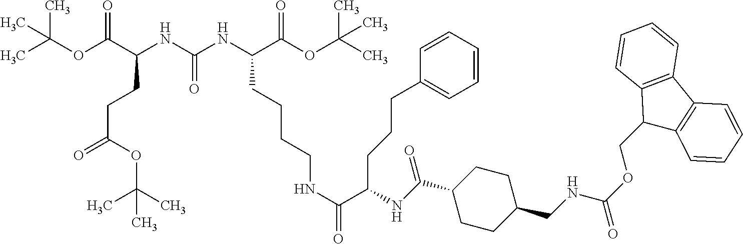 [SMILES: CC(C)(C)OC(=O)CC[C@H](NC(=O)N[C@@H](CCCCNC(=O)[C@H](CCCc1ccccc1)NC(=O)[C@H]1CC[C@H](CNC(=O)OCC2c3ccccc3-c3ccccc32)CC1)C(=O)OC(C)(C)C)C(=O)OC(C)(C)C]

di-tert-butyl (2S)-2-({[(2S)-6-{[(2S)-2-amino-5-phenylpentanoyl]amino}-1-tert-butoxy-1-oxohexan-2-yl]carbamoyl}amino)pentanedioate (199 mg, 300 μmol) and (1r,4r)-4-[({[(9H-fluoren-9-yl)methoxy]carbonyl}amino)methyl]cyclohexane-1-carboxylic acid (171 mg, 450 μmol) were solubilised in DMF (2.3 ml), 4-methylmorpholine (99 μl, 900 μmol, CAS-RN: 109-02-4) and HATU (171 mg, 450 μmol) were added and the mixture was stirred under argon overnight at rt.

The mixture was evaporated and purified by preparative HPLC (C18, acetonitrile/water with 0.1% formic acid) to give 250 mg (81% yield) of the target compound.

LC-MS (Method 1): Rₜ=1.66 min; MS (ESIpos): m/z=1025 [M+H]⁺

¹H-NMR (400 MHz, DMSO-d6) δ[ppm]: 1.230 (0.87), 1.379 (16.00), 1.383 (11.49), 1.387 (12.23), 1.668 (0.40), 1.683 (0.40), 2.074 (0.83), 2.518 (1.21), 2.522 (0.86), 2.728 (0.96), 2.888 (1.21), 4.288 (0.61), 4.305 (0.42), 7.139 (0.53), 7.158 (0.74), 7.244 (0.47), 7.263 (0.56), 7.281 (0.43), 7.316 (0.75), 7.319 (0.68), 7.335 (0.50), 7.338 (0.45), 7.407 (0.61), 7.679 (0.55), 7.698 (0.49), 7.877 (0.72), 7.896 (0.62), 8.158 (0.82).

Intermediate 16

### tri-tert-butyl (3S,10S,14S)-1-[(1r,4S)-4-(aminomethyl)cyclohexyl]-1,4,12-trioxo-3-(3-phenylpropyl)-2,5,11,13-tetraazahexadecane-10,14,16-tricarboxylate

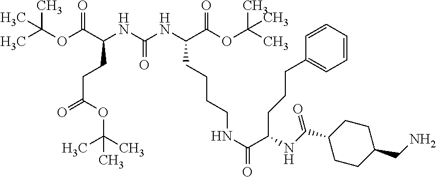 [SMILES: CC(C)(C)OC(=O)CC[C@H](NC(=O)N[C@@H](CCCCNC(=O)[C@H](CCCc1ccccc1)NC(=O)[C@H]1CC[C@H](CN)CC1)C(=O)OC(C)(C)C)C(=O)OC(C)(C)C]

tri-tert-butyl (3S,10S,14S)-1-{(1r,4S)-4-[({[(9H-fluoren-9-yl)methoxy]carbonyl}amino)methyl]cyclohexyl}-1,4,12-trioxo-3-(3-phenylpropyl)-2,5,11,13-tetraazahexadecane-10,14,16-tricarboxylate (250 mg, 244 μmol) was solubilised in DMF (6.4 ml), piperidine (480 μl, 4.9 mmol) was added and the mixture was stirred under argon overnight at rt. The mixture was evaporated and purified by preparative HPLC (C18, acetonitrile/water with 0.1% formic acid) to give 86.0 mg (44% yield) of the target compound.

LC-MS (Method 1): Rt=1.18 min; MS (ESIpos): m/z=803 [M+H]⁺

¹H-NMR (400 MHz, DMSO-d6) δ[ppm]: 1.383 (16.00), 1.385 (12.85), 1.390 (12.10), 2.518 (1.30), 2.523 (0.94), 2.540 (1.29), 2.559 (0.68), 7.137 (0.84), 7.157 (1.06), 7.244 (0.54), 7.261 (0.69), 8.408 (1.08).

### Example 3-A

### N⁶—{N-[(1r,4S)-4-(aminomethyl)cyclohexane-1-carbonyl]-5-phenyl-L-norvalyl}-N²-{[(1S)-1,3-dicarboxypropyl]carbamoyl}-L-lysine

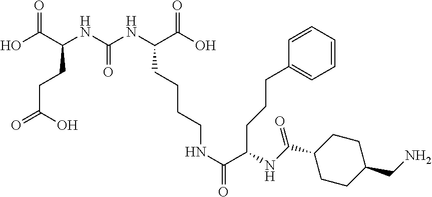 [SMILES: NC[C@H]1CC[C@H](C(=O)N[C@@H](CCCc2ccccc2)C(=O)NCCCC[C@H](NC(=O)N[C@@H](CCC(=O)O)C(=O)O)C(=O)O)CC1]

tri-tert-butyl (3S,10S,14S)-1-[(1r,4S)-4-(aminomethyl)cyclohexyl]-1,4,12-trioxo-3-(3-phenylpropyl)-2,5,11,13-tetraazahexadecane-10,14,16-tricarboxylate (13.4 mg, 98% purity, 16.4 μmol) was solubilised in DCM (1.1 ml), TFA (1.1 ml, 15 mmol) was added and the mixture was stirred under argon for 2 h at rt. The mixture was evaporated and purified by preparative HPLC (C18, acetonitrile/water with 0.1% formic acid) to give 6.00 mg (95% purity, 55% yield) of the target compound.

LC-MS (Method 1): Rₜ=0.70 min; MS (ESIpos): m/z=635 [M+H]⁺

¹H-NMR (400 MHz, DMSO-d6) δ[ppm]: 0.891 (1.19), 0.920 (1.72), 0.950 (1.32), 1.247 (3.11), 1.285 (2.45), 1.331 (3.37), 1.350 (2.91), 1.447 (1.85), 1.468 (2.71), 1.492 (3.44), 1.531 (1.92), 1.550 (2.45), 1.568 (2.78), 1.584 (2.51), 1.604 (2.38), 1.616 (2.25), 1.684 (1.45), 1.715 (3.44), 1.748 (2.12), 1.770 (1.59), 1.787 (1.65), 1.805 (1.98), 1.843 (0.79), 1.862 (0.46), 1.905 (0.79), 2.126 (1.45), 2.149 (2.38), 2.162 (2.25), 2.180 (1.39), 2.217 (0.99), 2.238 (1.52), 2.254 (0.86), 2.261 (0.99), 2.274 (0.93), 2.297 (0.46), 2.336 (1.19), 2.518 (16.00), 2.523 (12.36), 2.540 (12.43), 2.622 (2.45), 2.935 (0.86), 2.950 (1.19), 2.967 (1.52), 2.983 (1.32), 3.045 (1.45), 3.060 (1.72), 3.077 (1.52), 3.093 (1.12), 3.340 (4.96), 3.918 (2.18), 3.931 (2.78), 4.185 (1.39), 4.199 (1.59), 6.125 (1.06), 6.398 (1.65), 6.416 (1.65), 7.142 (5.36), 7.155 (5.36), 7.162 (7.47), 7.172 (3.24), 7.243 (6.21), 7.261 (6.28), 7.280 (2.91), 7.785 (1.26), 7.799 (2.31), 7.812 (1.26), 7.900 (2.12), 7.921 (2.05).

#4 Linker

Intermediate 17

### tri-tert-butyl (5S,12S,16S)-5-[3-(carbamoylamino)propyl]-1-(9H-fluoren-9-yl)-3,6,14-trioxo-2-oxa-4,7,13,15-tetraazaoctadecane-12,16,18-tricarboxylate

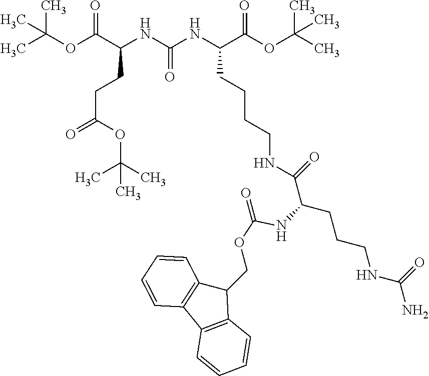 [SMILES: CC(C)(C)OC(=O)CC[C@H](NC(=O)N[C@@H](CCCCNC(=O)[C@H](CCCNC(N)=O)NC(=O)OCC1c2ccccc2-c2ccccc21)C(=O)OC(C)(C)C)C(=O)OC(C)(C)C]

di-tert-butyl N-{[(2S)-6-amino-1-tert-butoxy-1-oxohexan-2-yl]carbamoyl}-L-glutamate (300 mg, 70% purity, 431 μmol) and N5-carbamoyl-N2-{[(9H-fluoren-9-yl)methoxy]carbonyl}-L-ornithine (257 mg, 646 μmol) were solubilised in DMF (3.3 ml), 4-methylmorpholine (140 μl, 1.3 mmol, CAS-RN: 109-02-4) and HATU (246 mg, 646 μmol) were added and the mixture was stirred under argon for 3 h at rt. The mixture was evaporated and purified by preparative HPLC (C18, acetonitrile/water with 0.1% formic acid) to give 310 mg (92% purity, 76% yield) of the target compound.

LC-MS (Method 1): Rₜ=1.43 min; MS (ESIpos): m/z=868 [M+H]⁺

¹H-NMR (400 MHz, DMSO-d6) δ[ppm]: 1.380 (16.00), 1.384 (9.41), 2.297 (0.94), 2.686 (0.68), 2.729 (0.43), 2.888 (0.55), 4.207 (0.47), 5.383 (0.47), 7.324 (0.54), 7.414 (0.60), 7.880 (0.74), 7.898 (0.62).

Intermediate 18

### tri-tert-butyl (6S,13S,17S)-1,6-diamino-1,7,15-trioxo-2,8,14,16-tetraazanonadecane-13,17,19-tricarboxylate

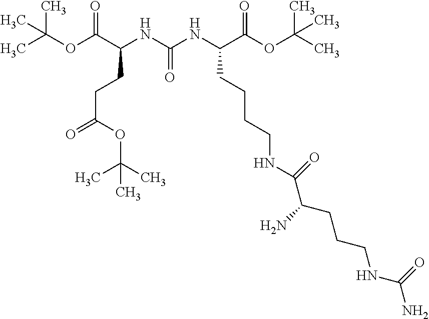 [SMILES: CC(C)(C)OC(=O)CC[C@H](NC(=O)N[C@@H](CCCCNC(=O)[C@@H](N)CCCNC(N)=O)C(=O)OC(C)(C)C)C(=O)OC(C)(C)C]

tri-tert-butyl (5S,12S,16S)-5-[3-(carbamoylamino)propyl]-1-(9H-fluoren-9-yl)-3,6,14-trioxo-2-oxa-4,7,13,15-tetraazaoctadecane-12,16,18-tricarboxylate (310 mg, 92% purity, 329 μmol) was solubilised in DMF (8.6 ml), piperidine (650 μl, 6.6 mmol) was added and the mixture was stirred under argon overnight at rt. The mixture was evaporated and purified by preparative HPLC (C18, acetonitrile/water with 0.1% formic acid) to give 212 mg (90% purity, 90% yield) of the target compound.

¹H-NMR (400 MHz, DMSO-d6) δ[ppm]: 1.385 (16.00), 1.392 (11.97), 1.397 (12.79), 2.297 (0.59), 2.518 (0.52), 5.397 (0.71), 8.286 (0.60).

Intermediate 19

### tri-tert-butyl (6S,13S,17S)-1-amino-6-({(1r,4S)-4-[({[(9H-fluoren-9-yl)methoxy]carbonyl}amino)methyl]cyclohexane-1-carbonyl}amino)-1,7,15-trioxo-2,8,14,16-tetraazanonadecane-13,17,19-tricarboxylate

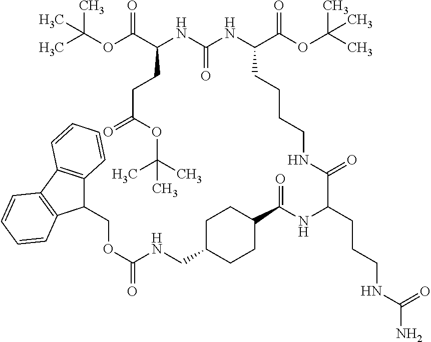

tri-tert-butyl (6S,13S,17S)-1,6-diamino-1,7,15-trioxo-2,8,14,16-tetraazanonadecane-13,17,19-tricarboxylate (212 mg, 329 μmol) and (1r,4r)-4-[({[(9H-fluoren-9-yl)methoxy]carbonyl}amino)methyl]cyclohexane-1-carboxylic acid (187 mg, 493 μmol) were solubilised in DMF (2.5 ml), 4-methylmorpholine (110 μl, 990 μmol, CAS-RN: 109-02-4) and HATU (188 mg, 493 μmol) were added and the mixture was stirred under argon overnight at rt. The mixture was evaporated and purified by preparative HPLC (C18, acetonitrile/water with 0.1% formic acid) to give 300 mg (90% purity, 82% yield) of the target compound.

¹H-NMR (400 MHz, DMSO-d6) δ[ppm]: 1.232 (0.64), 1.255 (0.74), 1.275 (0.64), 1.315 (0.45), 1.380 (14.07), 1.384 (16.00), 1.389 (9.61), 1.392 (12.09), 1.668 (0.59), 1.694 (0.54), 2.297 (5.78), 2.322 (0.41), 2.327 (0.57), 2.331 (0.46), 2.518 (2.70), 2.522 (1.71), 2.664 (0.41), 2.669 (0.54), 2.673 (0.41), 2.818 (0.59), 4.200 (0.41), 4.286 (0.75), 4.294 (0.65), 4.303 (0.53), 4.310 (0.41), 5.367 (0.82), 6.297 (0.41), 7.143 (0.64), 7.163 (1.30), 7.181 (1.49), 7.231 (1.27), 7.235 (0.46), 7.249 (1.36), 7.267 (0.59), 7.303 (0.67), 7.320 (1.21), 7.322 (1.21), 7.338 (0.83), 7.341 (0.81), 7.392 (0.69), 7.410 (1.10), 7.429 (0.49), 7.680 (0.99), 7.698 (0.89), 7.879 (1.25), 7.897 (1.14).

Intermediate 20

### formic acid-tri-tert-butyl (6S,13S,17S)-1-amino-6-{[(1r,4S)-4-(aminomethyl)cyclohexane-1-carbonyl]amino}-1,7,15-trioxo-2,8,14,16-tetraazanonadecane-13,17,19-tricarboxylate (1/1)

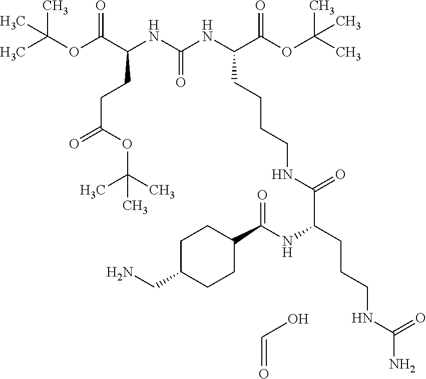

tri-tert-butyl (6S,13S,17S)-1-amino-6-({(1r,4S)-4-[({[(9H-fluoren-9-yl)methoxy]carbonyl}amino)methyl]cyclohexane-1-carbonyl}amino)-1,7,15-trioxo-2,8,14,16-tetraazanonadecane-13,17,19-tricarboxylate (300 mg, 90% purity, 268 μmol) was solubilised in DMF (7.0 ml), piperidine (530 μl, 5.4 mmol) was added and the mixture was stirred under argon for 4 h at rt. The mixture was evaporated and purified by preparative HPLC (C18, acetonitrile/water with 0.1% formic acid) to give 67.0 mg (98% purity, 29% yield) of the target compound.

LC-MS (Method 1): Rₜ=1.01 min; MS (ESIneg): m/z=829 [M−H]⁻

¹H-NMR (400 MHz, DMSO-d6) δ[ppm]: 1.273 (0.45), 1.359 (0.45), 1.385 (16.00), 1.388 (11.67), 1.395 (11.31), 2.518 (1.92), 2.523 (1.40), 2.534 (0.64), 2.551 (0.55), 5.382 (0.67), 8.412 (1.43).

### Example 4-A

### N⁶—{N²-[(1r,4S)-4-(aminomethyl)cyclohexane-1-carbonyl]-N⁵-carbamoyl-L-ornithyl}-N²-{[(1S)-1,3-dicarboxypropyl]carbamoyl}-L-lysine

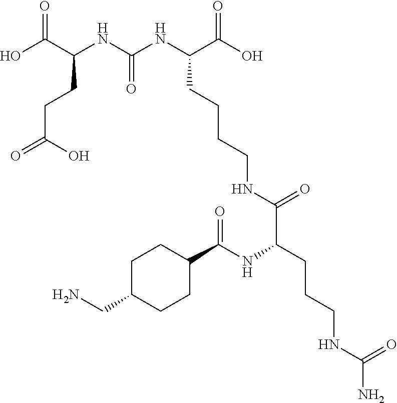

formic acid-tri-tert-butyl (6S,13S,17S)-1-amino-6-{[(1r,4S)-4-(aminomethyl)cyclohexane-1-carbonyl]amino}-1,7,15-trioxo-2,8,14,16-tetraazanonadecane-13,17,19-tricarboxylate (1/1) (20.0 mg, 98% purity, 23.6 μmol) was solubilised in DCM (1.4 ml), TFA (1.4 ml, 19 mmol) was added and the mixture was stirred under argon at rt. The mixture was evaporated and purified by preparative HPLC (C18, acetonitrile/water with 0.2% ammonia) to give 7.50 mg (95% purity, 49% yield) of the target compound.

¹H-NMR (400 MHz, DMSO-d6) δ[ppm]: 0.931 (0.66), 1.266 (1.93), 1.289 (1.64), 1.326 (1.39), 1.344 (1.37), 1.360 (1.34), 1.440 (0.79), 1.461 (1.10), 1.475 (1.13), 1.494 (0.90), 1.577 (0.85), 1.595 (0.83), 1.717 (1.60), 1.746 (2.38), 2.158 (0.69), 2.185 (1.12), 2.201 (1.30), 2.210 (1.30), 2.230 (0.62), 2.327 (0.40), 2.522 (1.38), 2.539 (16.00), 2.627 (1.25), 2.641 (1.24), 2.665 (0.52), 2.669 (0.56), 2.922 (1.29), 3.018 (1.48), 3.032 (1.48), 3.558 (1.17), 3.777 (0.86), 3.896 (0.88), 3.915 (1.22), 3.929 (1.27), 3.955 (1.26), 3.972 (1.15), 4.116 (0.60), 4.137 (0.92), 4.151 (0.91), 4.171 (0.56), 5.586 (0.77), 6.243 (0.59), 6.260 (0.58), 6.327 (0.81), 6.346 (0.78), 6.446 (0.42), 7.829 (0.55), 7.842 (1.00), 7.855 (0.55), 7.887 (0.91), 7.907 (0.87), 8.247 (1.28).

#5 Linker

Intermediate 21

### tri-tert-butyl (5S,12S,16S)-5-[4-(carbamoylamino)butyl]-1-(9H-fluoren-9-yl)-3,6,14-trioxo-2-oxa-4,7,13,15-tetraazaoctadecane-12,16,18-tricarboxylate

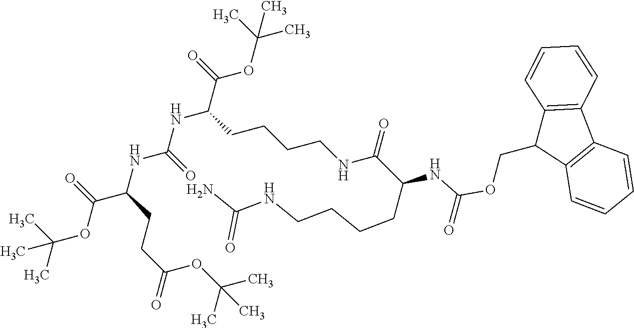 [SMILES: CC(C)(C)OC(=O)CC[C@H](NC(=O)N[C@@H](CCCCNC(=O)[C@H](CCCCNC(N)=O)NC(=O)OCC1c2ccccc2-c2ccccc21)C(=O)OC(C)(C)C)C(=O)OC(C)(C)C]

di-tert-butyl N-{[(2S)-6-amino-1-tert-butoxy-1-oxohexan-2-yl]carbamoyl}-L-glutamate (300 mg, 70% purity, 431 μmol) and N6-carbamoyl-N2-{[(9H-fluoren-9-yl)methoxy]carbonyl}-L-lysine (266 mg, 646 μmol) were solubilised in DMF (3.3 ml), 4-methylmorpholine (140 μl, 1.3 mmol, CAS-RN: 109-02-4) and HATU (246 mg, 646 μmol) were added and the mixture was stirred under argon overnight at rt. The mixture was evaporated and purified by preparative H PLC (C18, acetonitrile/water with 0.1% formic acid) to give 310 mg (98% purity, 80% yield) of the target compound.

LC-MS (Method 1): Rₜ=1.44 min; MS (ESIpos): m/z=882 [M+H]⁺

¹H-NMR (400 MHz, DMSO-d6) δ[ppm]: 1.380 (16.00), 1.384 (8.62), 2.518 (0.45), 4.212 (0.49), 5.346 (0.40), 7.322 (0.47), 7.324 (0.46), 7.414 (0.62), 7.881 (0.65), 7.900 (0.55).

Intermediate 22

### tri-tert-butyl (7S,14S,18S)-1,7-diamino-1,8,16-trioxo-2,9,15,17-tetraazaicosane-14,18,20-tricarboxylate

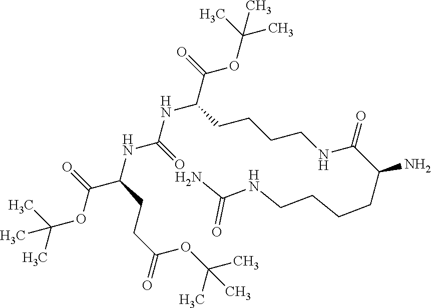 [SMILES: CC(C)(C)OC(=O)CC[C@H](NC(=O)N[C@@H](CCCCNC(=O)[C@@H](N)CCCCNC(N)=O)C(=O)OC(C)(C)C)C(=O)OC(C)(C)C]

tri-tert-butyl (5S,12S,16S)-5-[4-(carbamoylamino)butyl]-1-(9H-fluoren-9-yl)-3,6,14-trioxo-2-oxa-4,7,13,15-tetraazaoctadecane-12,16,18-tricarboxylate (210 mg, 98% purity, 234 μmol) was solubilised in DMF (2.5 ml), piperidine (460 μl, 4.7 mmol) was added and the mixture was stirred under argon overnight at rt. The mixture was evaporated and purified by preparative HPLC (C18, acetonitrile/water with 0.1% formic acid) to give 131 mg (85% purity, 72% yield) of the target compound.

¹H-NMR (400 MHz, DMSO-d6) δ[ppm]: 1.272 (0.77), 1.289 (0.78), 1.310 (0.76), 1.327 (0.69), 1.385 (16.00), 1.391 (14.38), 1.396 (14.61), 2.907 (0.62), 2.922 (0.61), 3.051 (0.41), 3.242 (0.41), 3.951 (0.41), 5.372 (0.89), 6.295 (0.47), 6.316 (0.46), 6.339 (0.49), 6.360 (0.46), 8.289 (1.02).

Intermediate 23 tri-tert-butyl (7S,14S,18S)-1-amino-7-({(1r,4S)-4-[({[(9H-fluoren-9-yl)methoxy]carbonyl}amino)methyl]cyclohexane-1-carbonyl}amino)-1,8,16-trioxo-2,9,15,17-tetraazaicosane-14,18,20-tricarboxylate

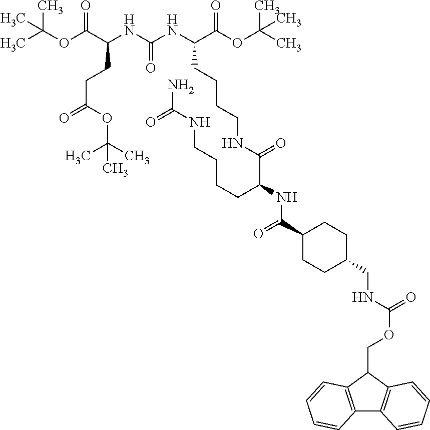 [SMILES: CC(C)(C)OC(=O)CC[C@H](NC(=O)N[C@@H](CCCCNC(=O)[C@H](CCCCNC(N)=O)NC(=O)[C@H]1CC[C@H](CNC(=O)OCC2c3ccccc3-c3ccccc32)CC1)C(=O)OC(C)(C)C)C(=O)OC(C)(C)C]

tri-tert-butyl (7S,14S,18S)-1,7-diamino-1,8,16-trioxo-2,9,15,17-tetraazaicosane-14,18,20-tricarboxylate (180 mg, 273 μmol) and (1r,4r)-4-[({[(9H-fluoren-9-yl)methoxy]carbonyl}amino)methyl]cyclohexane-1-carboxylic acid (156 mg, 410 μmol) were solubilised in DMF (2.1 ml), 4-methylmorpholine (90 μl, 820 μmol, CAS-RN: 109-02-4) and HATU (156 mg, 410 μmol) were added and the mixture was stirred under argon at rt. The mixture was evaporated and purified by preparative HPLC (C18, acetonitrile/water with 0.1% formic acid) to give 119 mg (98% purity, 42% yield) of the target compound.

¹H-NMR (400 MHz, DMSO-d6) δ[ppm]: 1.231 (0.59), 1.252 (0.71), 1.269 (0.63), 1.289 (0.72), 1.315 (0.58), 1.343 (0.41), 1.380 (16.00), 1.384 (15.33), 1.391 (12.70), 1.667 (0.55), 1.702 (0.62), 2.297 (0.68), 2.821 (0.52), 2.893 (0.46), 2.908 (0.47), 4.286 (0.86), 4.304 (0.60), 5.342 (0.95), 6.245 (0.41), 6.290 (0.44), 6.310 (0.42), 7.303 (0.57), 7.321 (1.01), 7.340 (0.66), 7.392 (0.60), 7.410 (0.96), 7.429 (0.42), 7.681 (0.85), 7.699 (0.78), 7.879 (1.08), 7.898 (0.98).

Intermediate 24 tri-tert-butyl (7S,14S,18S)-1-amino-7-{[(1r,4S)-4-(aminomethyl)cyclohexane-1-carbonyl]amino}-1,8,16-trioxo-2,9,15,17-tetraazaicosane-14,18,20-tricarboxylate

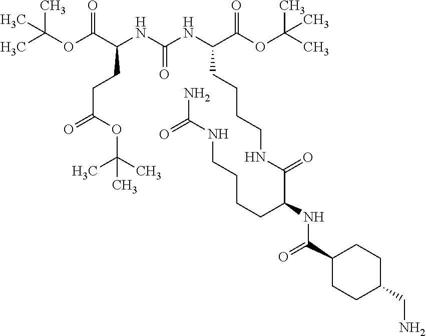 [SMILES: CC(C)(C)OC(=O)CC[C@H](NC(=O)N[C@@H](CCCCNC(=O)[C@H](CCCCNC(N)=O)NC(=O)[C@H]1CC[C@H](CN)CC1)C(=O)OC(C)(C)C)C(=O)OC(C)(C)C]

tri-tert-butyl (7S,14S,18S)-1-amino-7-({(1r,4S)-4-[({[(9H-fluoren-9-yl)methoxy]carbonyl}amino)methyl]cyclohexane-1-carbonyl}amino)-1,8,16-trioxo-2,9,15,17-tetraazaicosane-14,18,20-tricarboxylate (180 mg, 70% purity, 123 μmol) was solubilised in DMF (3.2 ml), piperidine (240 μl, 2.5 mmol) was added and the mixture was stirred under argon for 4 h at rt. The mixture was evaporated and purified by preparative HPLC (C18, acetonitrile/water with 0.1% formic acid) to give 94.0 mg (85% purity, 81% yield) of the target compound.

LC-MS (Method 1): Rₜ=1.00 min; MS (ESIpos): m/z=799 [M+H]⁺

¹H-NMR (400 MHz, DMSO-d6) δ[ppm]: 1.249 (0.41), 1.270 (0.42), 1.286 (0.43), 1.312 (0.41), 1.358 (0.41), 1.384 (16.00), 1.387 (11.85), 1.394 (11.13), 2.518 (1.20), 2.522 (0.82), 2.536 (0.52), 2.539 (0.42), 2.552 (0.50), 3.350 (0.48), 5.357 (0.64), 8.421 (0.62).

### Example 5-A

N⁶—{N²-[(1r,4S)-4-(aminomethyl)cyclohexane-1-carbonyl]-N⁶-carbamoyl-L-lysyl}-N²-{[(1S)-1,3-dicarboxypropyl]carbamoyl}-L-lysine

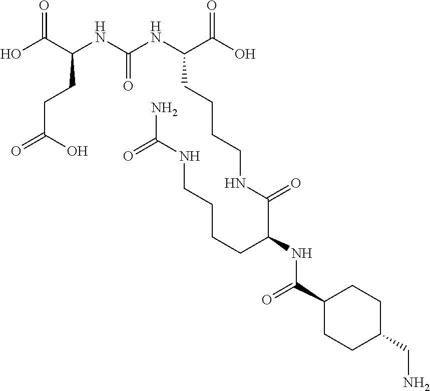 [SMILES: NC[C@H]1CC[C@H](C(=O)N[C@@H](CCCCNC(N)=O)C(=O)NCCCC[C@H](NC(=O)N[C@@H](CCC(=O)O)C(=O)O)C(=O)O)CC1]

tri-tert-butyl (7S,14S,18S)-1-amino-7-{[(1r,4S)-4-(aminomethyl)cyclohexane-1-carbonyl]amino}-1,8,16-trioxo-2,9,15,17-tetraazaicosane-14,18,20-tricarboxylate (30.0 mg, 98% purity, 36.8 μmol) was solubilised in DCM (2.3 ml), TFA (2.3 ml, 29 mmol) was added and the mixture was stirred under argon at rt. The mixture was evaporated and purified by preparative HPLC (C18, acetonitrile/water with 0.1% formic acid) to give 5.00 mg (90% purity, 19% yield) of the target compound.

¹H-NMR (400 MHz, DMSO-d6) δ[ppm]: 0.929 (0.98), 1.263 (2.69), 1.290 (2.43), 1.333 (2.33), 1.473 (1.14), 1.556 (1.40), 1.710 (1.61), 1.738 (1.76), 1.796 (1.19), 2.074 (1.24), 2.136 (1.09), 2.155 (1.09), 2.219 (1.04), 2.518 (11.29), 2.523 (7.87), 2.540 (4.25), 2.620 (1.24), 2.876 (1.04), 2.945 (1.35), 2.961 (1.29), 2.993 (1.35), 3.072 (1.61), 3.354 (16.00), 3.882 (1.19), 3.927 (1.14), 4.108 (1.09), 4.122 (1.09), 5.537 (2.38), 6.162 (0.62), 6.370 (1.45), 7.781 (1.24), 7.930 (0.98), 7.948 (1.04).

#6 Linker

Intermediate 25

### tri-tert-butyl (5S,12S,16S)-1-(9H-fluoren-9-yl)-3,6,14-trioxo-5-[(3,4,5-trifluorophenyl)methyl]-2-oxa-4,7,13,15-tetraazaoctadecane-12,16,18-tricarboxylate

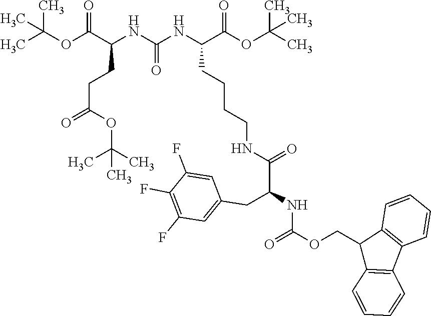 [SMILES: CC(C)(C)OC(=O)CC[C@H](NC(=O)N[C@@H](CCCCNC(=O)[C@H](Cc1cc(F)c(F)c(F)c1)NC(=O)OCC1c2ccccc2-c2ccccc21)C(=O)OC(C)(C)C)C(=O)OC(C)(C)C]

di-tert-butyl N-{[(2S)-6-amino-1-tert-butoxy-1-oxohexan-2-yl]carbamoyl}-L-glutamate (300 mg, 70% purity, 431 μmol) and N-{[(9H-fluoren-9-yl)methoxy]carbonyl}-3,4,5-trifluoro-L-phenylalanine (285 mg, 646 μmol; CAS-RN:[205526-30-3]) were solubilised in DMF (3.3 ml), 4-methylmorpholine (140 μl, 1.3 mmol, CAS-RN: 109-02-4) and HATU (246 mg, 646 μmol) were added and the mixture was stirred under argon at rt. The mixture was evaporated and purified by preparative HPLC (C18, acetonitrile/water with 0.1% formic acid) to give 310 mg (95% purity, 75% yield) of the target compound.

LC-MS (Method 1): Rₜ=1.66 min; MS (ESIpos): m/z=912 [M+H]⁺

Intermediate 26

### di-tert-butyl (2S)-2-({[(2S)-6-{[(2S)-2-amino-3-(3,4,5-trifluorophenyl)propanoyl]amino}-1-tert-butoxy-1-oxohexan-2-yl]carbamoyl}amino)pentanedioate

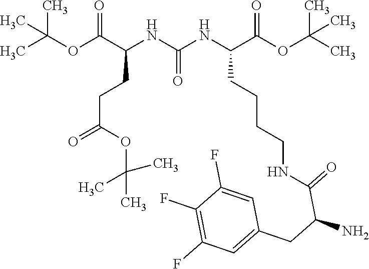 [SMILES: CC(C)(C)OC(=O)CC[C@H](NC(=O)N[C@@H](CCCCNC(=O)[C@@H](N)Cc1cc(F)c(F)c(F)c1)C(=O)OC(C)(C)C)C(=O)OC(C)(C)C]

tri-tert-butyl (5S,12S,16S)-1-(9H-fluoren-9-yl)-3,6,14-trioxo-5-[(3,4,5-trifluorophenyl)methyl]-2-oxa-4,7,13,15-tetraazaoctadecane-12,16,18-tricarboxylate (310 mg, 95% purity, 323 μmol) was solubilised in DMF (3.5 ml), piperidine (640 μl, 6.5 mmol) was added and the mixture was stirred under argon overnight at rt. The mixture was evaporated and purified by preparative HPLC (C18, acetonitrile/water with 0.1% formic acid) to give 243 mg of the target compound.

LC-MS (Method 1): Rₜ=1.02 min; MS (ESIpos): m/z=690 [M+H]⁺

Intermediate 27

### tri-tert-butyl (3S,10S,14S)-1-{(1r,4S)-4-[({[(9H-fluoren-9-yl)methoxy]carbonyl}amino)methyl]cyclohexyl}-1,4,12-trioxo-3-[(3,4,5-trifluorophenyl)methyl]-2,5,11,13-tetraazahexadecane-10,14,16-tricarboxylate

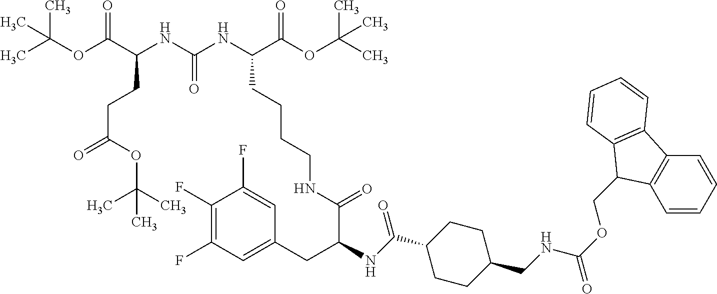 [SMILES: CC(C)(C)OC(=O)CC[C@H](NC(=O)N[C@@H](CCCCNC(=O)[C@H](Cc1cc(F)c(F)c(F)c1)NC(=O)[C@H]1CC[C@H](CNC(=O)OCC2c3ccccc3-c3ccccc32)CC1)C(=O)OC(C)(C)C)C(=O)OC(C)(C)C]

di-tert-butyl (2S)-2-({[(2S)-6-{[(2S)-2-amino-3-(3,4,5-trifluorophenyl)propanoyl]amino}-1-tert-butoxy-1-oxohexan-2-yl]carbamoyl}amino)pentanedioate (243 mg, 92% purity, 325 μmol) and (1 r,4r)-4-[({[(9H-fluoren-9-yl)methoxy]carbonyl}amino)methyl]cyclohexane-1-carboxylic acid (185 mg, 487 μmol) were solubilised in DMF (2.5 ml), 4-methylmorpholine (110 μl, 970 μmol, CAS-RN: 109-02-4) and HATU (185 mg, 487 μmol) were added and the mixture was stirred under argon overnight at rt. The mixture was evaporated and purified by preparative HPLC (C18, acetonitrile/water with 0.1% formic acid) to give 250 mg (98% purity, 72% yield) of the target compound.

LC-MS (Method 1): Rₜ=1.65 min; MS (ESIpos): m/z=1051 [M+H]⁺

¹H-NMR (400 MHz, DMSO-d6) δ[ppm]: 1.379 (16.00), 1.384 (13.65), 1.387 (14.51), 2.518 (1.40), 2.523 (0.90), 2.728 (3.65), 2.888 (4.36), 4.289 (0.67), 4.305 (0.46), 7.312 (0.78), 7.315 (0.73), 7.331 (0.51), 7.387 (0.42), 7.406 (0.63), 7.675 (0.62), 7.694 (0.55), 7.877 (0.70), 7.896 (0.65), 7.938 (0.47), 7.951 (0.68), 8.162 (0.61).

Intermediate 28

### tri-tert-butyl (3S,10S,14S)-1-[(1r,4S)-4-(aminomethyl)cyclohexyl]-1,4,12-trioxo-3-[(3,4,5-trifluorophenyl)methyl]-2,5,11,13-tetraazahexadecane-10,14,16-tricarboxylate

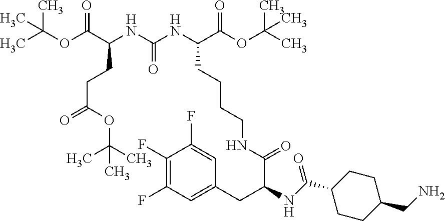 [SMILES: CC(C)(C)OC(=O)CC[C@H](NC(=O)N[C@@H](CCCCNC(=O)[C@H](Cc1cc(F)c(F)c(F)c1)NC(=O)[C@H]1CC[C@H](CN)CC1)C(=O)OC(C)(C)C)C(=O)OC(C)(C)C]

tri-tert-butyl (3S,10S,14S)-1-{(1r,4S)-4-[({[(9H-fluoren-9-yl)methoxy]carbonyl}amino)methyl]cyclohexyl}-1,4,12-trioxo-3-[(3,4,5-trifluorophenyl)methyl]-2,5,11,13-tetraazahexadecane-10,14,16-tricarboxylate (250 mg, 98% purity, 233 μmol) was solubilised in DMF (6.1 ml), piperidine (460 μl, 4.7 mmol) was added and the mixture was stirred under argon overnight at rt. The mixture was evaporated and purified by preparative HPLC (C18, acetonitrile/water with 0.1% formic acid) to give 96.0 mg (98% purity, 49% yield) of the target compound.

LC-MS (Method 1): Rₜ=1.17 min; MS (ESIpos): m/z=829 [M+H]⁺

¹H-NMR (400 MHz, DMSO-d6) δ[ppm]: 1.244 (0.43), 1.341 (0.42), 1.382 (14.92), 1.387 (16.00), 2.298 (2.72), 2.536 (0.82), 2.553 (0.65), 7.124 (0.44), 7.142 (0.70), 7.163 (0.97), 7.181 (0.76), 7.231 (0.59), 7.249 (0.66), 7.988 (0.45), 8.010 (0.44), 8.404 (1.15).

### Example 6-A

### N⁶—{N-[(1r,4S)-4-(aminomethyl)cyclohexane-1-carbonyl]-3,4,5-trifluoro-L-phenylalanyl}-N²-{[(1S)-1,3-dicarboxypropyl]carbamoyl}-L-lysine

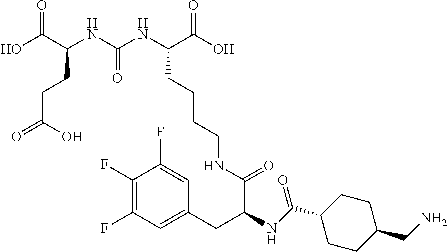 [SMILES: NC[C@H]1CC[C@H](C(=O)N[C@@H](Cc2cc(F)c(F)c(F)c2)C(=O)NCCCC[C@H](NC(=O)N[C@@H](CCC(=O)O)C(=O)O)C(=O)O)CC1]

tri-tert-butyl (3S,10S,14S)-1-[(1r,4S)-4-(aminomethyl)cyclohexyl]-1,4,12-trioxo-3-[(3,4,5-trifluorophenyl)methyl]-2,5,11,13-tetraazahexadecane-10,14,16-tricarboxylate (98.0 mg, 98% purity, 116 μmol) was solubilised in DCM (7.1 ml), TFA (7.1 ml, 92 mmol) was added and the mixture was stirred under argon at rt. The mixture was evaporated and purified by preparative HPLC (C18, acetonitrile/water with 0.1% formic acid) to give 21.0 mg (85% purity, 23% yield) of the target compound.

LC-MS (Method 1): Rₜ=0.70 min; MS (ESIpos): m/z=661 [M+H]⁺

¹H-NMR (400 MHz, DMSO-d6) δ[ppm]: 0.852 (1.11), 0.884 (2.74), 0.915 (3.04), 0.947 (1.26), 1.085 (0.74), 1.109 (1.63), 1.116 (1.63), 1.148 (1.63), 1.175 (1.19), 1.200 (1.78), 1.207 (1.78), 1.238 (3.85), 1.259 (3.78), 1.277 (3.56), 1.340 (3.33), 1.357 (3.63), 1.375 (2.37), 1.395 (1.41), 1.435 (2.67), 1.449 (2.96), 1.470 (2.30), 1.517 (1.93), 1.551 (1.70), 1.581 (2.22), 1.594 (3.26), 1.614 (3.33), 1.627 (2.15), 1.648 (2.37), 1.691 (3.11), 1.734 (2.52), 1.768 (2.15), 1.784 (1.93), 1.802 (1.56), 1.808 (1.63), 1.825 (1.56), 1.841 (1.11), 1.860 (0.59), 1.905 (1.11), 2.055 (1.26), 2.074 (3.11), 2.084 (2.30), 2.118 (1.56), 2.131 (1.33), 2.137 (1.26), 2.153 (2.07), 2.167 (2.07), 2.172 (2.00), 2.184 (1.33), 2.227 (1.56), 2.248 (2.22), 2.262 (1.33), 2.271 (1.63), 2.284 (1.33), 2.306 (0.81), 2.336 (1.41), 2.518 (16.00), 2.523 (11.11), 2.539 (0.89), 2.599 (7.26), 2.616 (7.19), 2.678 (1.41), 2.733 (1.70), 2.760 (2.22), 2.767 (2.59), 2.793 (2.30), 2.927 (1.33), 2.947 (3.33), 2.960 (4.37), 2.981 (2.89), 2.993 (2.30), 3.093 (2.00), 3.109 (2.59), 3.126 (2.30), 3.143 (1.85), 3.336 (4.22), 3.920 (2.00), 3.940 (4.07), 3.953 (4.15), 3.972 (1.85), 4.390 (1.26), 4.402 (1.41), 4.416 (2.00), 4.425 (2.07), 4.438 (1.41), 4.450 (1.26), 6.131 (1.48), 6.384 (2.67), 6.404 (2.59), 7.149 (4.52), 7.166 (5.19), 7.172 (5.19), 7.189 (4.74), 7.929 (1.85), 7.943 (3.41), 7.957 (1.78), 8.156 (3.04), 8.177 (2.89), 8.251 (0.74).

#7 Linker

Intermediate 29

### tri-tert-butyl (5S,12S,16S)-1-(9H-fluoren-9-yl)-5-[(4-fluorophenyl)methyl]-3,6,14-trioxo-2-oxa-4,7,13,15-tetraazaoctadecane-12,16,18-tricarboxylate

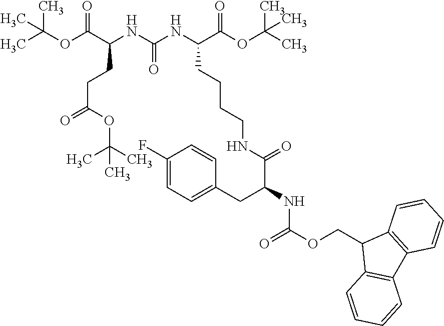 [SMILES: CC(C)(C)OC(=O)CC[C@H](NC(=O)N[C@@H](CCCCNC(=O)[C@H](Cc1ccc(F)cc1)NC(=O)OCC1c2ccccc2-c2ccccc21)C(=O)OC(C)(C)C)C(=O)OC(C)(C)C]

di-tert-butyl N-{[(2S)-6-amino-1-tert-butoxy-1-oxohexan-2-yl]carbamoyl}-L-glutamate (287 mg, 70% purity, 412 μmol) and N-{[(9H-fluoren-9-yl)methoxy]carbonyl}-4-fluoro-L-phenylalanine (251 mg, 618 μmol) were solubilised in DMF (3.2 ml), 4-methylmorpholine (140 μl, 1.2 mmol, CAS-RN: 109-02-4) and HATU (235 mg, 618 μmol) were added and the mixture was stirred under argon overnight at rt. The mixture was evaporated and purified by preparative HPLC (C18, acetonitrile/water with 0.1% formic acid) to give 280 mg (95% purity, 74% yield) of the target compound.

LC-MS (Method 1): Rₜ=1.63 min; MS (ESIpos): m/z=876 [M+H]⁺

¹H-NMR (400 MHz, DMSO-d6) δ[ppm]: 1.242 (0.63), 1.379 (16.00), 2.221 (0.48), 2.296 (0.73), 4.145 (1.16), 6.273 (0.61), 7.062 (0.63), 7.296 (1.17), 7.405 (0.73), 7.629 (0.70), 7.870 (0.72), 7.886 (0.74).

Intermediate 30

### di-tert-butyl (2S)-2-({[(2S)-6-{[(2S)-2-amino-3-(4-fluorophenyl)propanoyl]amino}-1-tert-butoxy-1-oxohexan-2-yl]carbamoyl}amino)pentanedioate

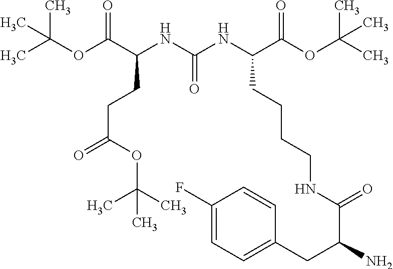 [SMILES: CC(C)(C)OC(=O)CC[C@H](NC(=O)N[C@@H](CCCCNC(=O)[C@@H](N)Cc1ccc(F)cc1)C(=O)OC(C)(C)C)C(=O)OC(C)(C)C]

tri-tert-butyl (5S,12S,16S)-1-(9H-fluoren-9-yl)-5-[(4-fluorophenyl)methyl]-3,6,14-trioxo-2-oxa-4,7,13,15-tetraazaoctadecane-12,16,18-tricarboxylate (280 mg, 95% purity, 304 μmol) was solubilised in DMF (8.0 ml), piperidine (600 μl, 6.1 mmol) was added and the mixture was stirred under argon at rt. The mixture was evaporated and purified by preparative HPLC (C18, acetonitrile/water with 0.1% formic acid) to give 160 mg (80% purity, 65% yield) of the target compound.

LC-MS (Method 1): Rₜ=1.10 min; MS (ESIpos): m/z=653 [M+H]⁺

Intermediate 31

### tri-tert-butyl (3S,10S,14S)-1-{(1r,4S)-4-[({[(9H-fluoren-9-yl)methoxy]carbonyl}amino)methyl]cyclohexyl}-3-[(4-fluorophenyl)methyl]-1,4,12-trioxo-2,5,11,13-tetraazahexadecane-10,14,16-tricarboxylate

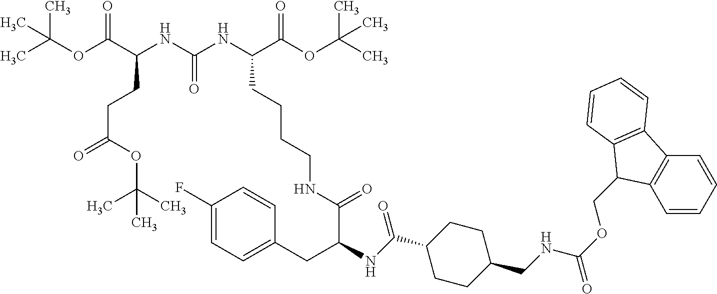 [SMILES: CC(C)(C)OC(=O)CC[C@H](NC(=O)N[C@@H](CCCCNC(=O)[C@H](Cc1ccc(F)cc1)NC(=O)[C@H]1CC[C@H](CNC(=O)OCC2c3ccccc3-c3ccccc32)CC1)C(=O)OC(C)(C)C)C(=O)OC(C)(C)C]

di-tert-butyl (2S)-2-({[(2S)-6-{[(2S)-2-amino-3-(4-fluorophenyl)propanoyl]amino}-1-tert-butoxy-1-oxohexan-2-yl]carbamoyl}amino)pentanedioate (160 mg, 245 μmol) and (1r,4r)-4-[({[(9H-fluoren-9-yl)methoxy]carbonyl}amino)methyl]cyclohexane-1-carboxylic acid (140 mg, 368 μmol) were solubilised in DMF (1.9 ml), 4-methylmorpholine (81 μl, 740 μmol, CAS-RN: 109-02-4) and HATU (140 mg, 368 μmol) were added and the mixture was stirred under argon overnight at rt. The mixture was evaporated and purified by preparative HPLC (C18, acetonitrile/water with 0.1% formic acid) to give 150 mg (60% yield) of the target compound.

LC-MS (Method 1): Rₜ=1.62 min; MS (ESIpos): m/z=1015 [M+H]⁺

¹H-NMR (400 MHz, DMSO-d6) δ[ppm]: 1.228 (0.43), 1.380 (16.00), 1.385 (13.83), 1.387 (15.17), 1.631 (0.44), 2.298 (1.35), 2.518 (1.75), 2.522 (1.15), 4.285 (0.72), 4.302 (0.49), 7.067 (0.65), 7.090 (0.41), 7.225 (0.47), 7.231 (0.69), 7.247 (0.62), 7.314 (0.79), 7.333 (0.52), 7.389 (0.44), 7.408 (0.68), 7.675 (0.65), 7.694 (0.58), 7.878 (0.82), 7.897 (0.71).

Intermediate 32

### tri-tert-butyl (3S,10S,14S)-1-[(1r,4S)-4-(aminomethyl)cyclohexyl]-3-[(4-fluorophenyl)methyl]-1,4,12-trioxo-2,5,11,13-tetraazahexadecane-10,14,16-tricarboxylate

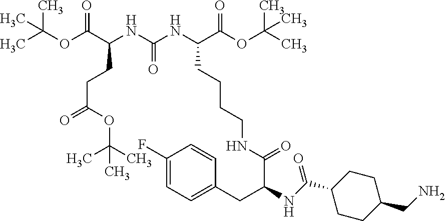 [SMILES: CC(C)(C)OC(=O)CC[C@H](NC(=O)N[C@@H](CCCCNC(=O)[C@H](Cc1ccc(F)cc1)NC(=O)[C@H]1CC[C@H](CN)CC1)C(=O)OC(C)(C)C)C(=O)OC(C)(C)C]

tri-tert-butyl (3S,10S,14S)-1-{(1r,4S)-4-[({[(9H-fluoren-9-yl)methoxy]carbonyl}amino)methyl]cyclohexyl}-3-[(4-fluorophenyl)methyl]-1,4,12-trioxo-2,5,11,13-tetraazahexadecane-10,14,16-tricarboxylate (150 mg, 80% purity, 118 μmol) was solubilised in DMF (3.1 ml), piperidine (230 μl, 2.4 mmol) was added and the mixture was stirred under argon overnight at rt. The mixture was evaporated and purified by preparative HPLC (C18, acetonitrile/water with 0.1% formic acid) to give 62.0 mg (66% yield) of the target compound.

LC-MS (Method 1): Rₜ=1.17 min; MS (ESIpos): m/z=793 [M+H]⁺

¹H-NMR (400 MHz, DMSO-d6) δ[ppm]: 1.347 (0.40), 1.383 (16.00), 1.387 (14.26), 1.389 (14.33), 2.518 (1.45), 2.523 (1.07), 2.527 (0.86), 7.039 (0.42), 7.062 (0.83), 7.084 (0.49), 7.208 (0.44), 7.222 (0.52), 7.229 (0.44), 8.421 (1.47).

### Example 7-A

### N⁶—{N-[(1r,4S)-4-(aminomethyl)cyclohexane-1-carbonyl]-4-fluoro-L-phenylalanyl}-N²-{[(1S)-1,3-dicarboxypropyl]carbamoyl}-L-lysine

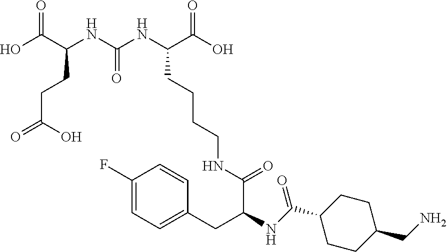 [SMILES: NC[C@H]1CC[C@H](C(=O)N[C@@H](Cc2ccc(F)cc2)C(=O)NCCCC[C@H](NC(=O)N[C@@H](CCC(=O)O)C(=O)O)C(=O)O)CC1]

tri-tert-butyl (3S,10S,14S)-1-[(1r,4S)-4-(aminomethyl)cyclohexyl]-3-[(4-fluorophenyl)methyl]-1,4,12-trioxo-2,5,11,13-tetraazahexadecane-10,14,16-tricarboxylate (13.8 mg, 98% purity, 17.1 μmol) was solubilised in DCM (1.0 ml), TFA (1.0 ml, 14 mmol) was added and the mixture was stirred under argon at rt. The mixture was evaporated and purified by preparative HPLC (C18, acetonitrile/water with 0.1% formic acid) to give 3.00 mg (98% purity, 28% yield) of the target compound.

LC-MS (Method 1): Rₜ=0.64 min; MS (ESIpos): m/z=625 [M+H]⁺

¹H-NMR (400 MHz, DMSO-d6) δ[ppm]: 0.868 (1.44), 0.898 (1.52), 0.924 (0.72), 1.096 (0.40), 1.128 (0.96), 1.161 (0.96), 1.204 (1.36), 1.239 (2.88), 1.261 (2.16), 1.320 (2.00), 1.337 (2.16), 1.352 (1.68), 1.437 (1.44), 1.452 (1.44), 1.470 (1.04), 1.504 (1.20), 1.541 (1.52), 1.569 (1.60), 1.686 (1.60), 1.751 (1.04), 1.777 (1.12), 1.808 (0.96), 1.834 (0.88), 1.898 (0.40), 2.049 (0.72), 2.074 (2.48), 2.117 (1.04), 2.139 (1.04), 2.153 (1.12), 2.223 (0.72), 2.248 (0.96), 2.268 (0.80), 2.302 (0.40), 2.332 (3.28), 2.336 (1.44), 2.518 (16.00), 2.523 (11.28), 2.539 (2.00), 2.572 (2.16), 2.586 (2.08), 2.678 (1.44), 2.729 (0.96), 2.755 (1.28), 2.763 (1.44), 2.789 (1.28), 2.917 (1.84), 2.928 (2.08), 2.951 (2.16), 2.962 (1.84), 3.062 (1.44), 3.076 (1.68), 3.093 (1.60), 3.110 (1.44), 3.351 (4.48), 3.867 (1.36), 3.881 (1.44), 3.942 (1.36), 4.356 (0.72), 4.379 (1.28), 4.392 (1.28), 4.402 (0.80), 4.415 (0.72), 6.148 (0.64), 6.333 (1.12), 6.352 (1.04), 7.036 (3.12), 7.058 (6.24), 7.080 (3.68), 7.217 (3.20), 7.231 (3.84), 7.238 (3.28), 7.252 (2.48), 7.913 (1.52), 8.147 (1.20), 8.169 (1.20), 8.379 (1.04).

#8 Linker

Intermediate 33

### tri-tert-butyl (5S,12S,16S)-5-[(4-tert-butylphenyl)methyl]-1-(9H-fluoren-9-yl)-3,6,14-trioxo-2-oxa-4,7,13,15-tetraazaoctadecane-12,16,18-tricarboxylate

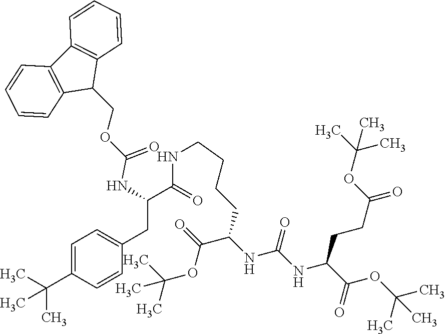 [SMILES: CC(C)(C)OC(=O)CC[C@H](NC(=O)N[C@@H](CCCCNC(=O)[C@H](Cc1ccc(C(C)(C)C)cc1)NC(=O)OCC1c2ccccc2-c2ccccc21)C(=O)OC(C)(C)C)C(=O)OC(C)(C)C]

di-tert-butyl N-{[(2S)-6-amino-1-tert-butoxy-1-oxohexan-2-yl]carbamoyl}-L-glutamate (300 mg, 70% purity, 431 μmol) and 4-tert-butyl-N-{[(9H-fluoren-9-yl)methoxy]carbonyl}-L-phenylalanine (287 mg, 646 μmol) were solubilised in DMF (3.3 ml), 4-methylmorpholine (140 μl, 1.3 mmol, CAS-RN: 109-02-4) and HATU (246 mg, 646 μmol) were added and the mixture was stirred under argon at rt. The mixture was evaporated and purified by preparative HPLC (C18, acetonitrile/water with 0.1% formic acid) to give 340 mg (98% purity, 85% yield) of the target compound.

LC-MS (Method 1): Rt=1.75 min; MS (ESIpos): m/z=914 [M+H]⁺

¹H-NMR (400 MHz, DMSO-d6) δ[ppm]: 1.180 (5.18), 1.235 (0.77), 1.381 (16.00), 2.297 (1.09), 4.073 (0.57), 6.275 (0.58), 7.168 (0.60), 7.189 (0.94), 7.218 (0.86), 7.231 (0.58), 7.249 (0.52), 7.274 (0.46), 7.293 (0.41), 7.395 (0.54), 7.411 (0.46), 7.865 (0.67), 7.883 (0.61).

Intermediate 34

### di-tert-butyl (2S)-2-({[(2S)-6-{[(2S)-2-amino-3-(4-tert-butylphenyl)propanoyl]amino}-1-tert-butoxy-1-oxohexan-2-yl]carbamoyl}amino)pentanedioate

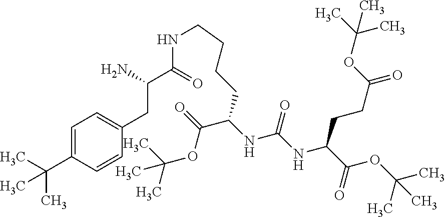 [SMILES: CC(C)(C)OC(=O)CC[C@H](NC(=O)N[C@@H](CCCCNC(=O)[C@@H](N)Cc1ccc(C(C)(C)C)cc1)C(=O)OC(C)(C)C)C(=O)OC(C)(C)C]

tri-tert-butyl (5S,12S,16S)-5-[(4-tert-butylphenyl)methyl]-1-(9H-fluoren-9-yl)-3,6,14-trioxo-2-oxa-4,7,13,15-tetraazaoctadecane-12,16,18-tricarboxylate (340 mg, 98% purity, 365 μmol) was solubilised in DMF (9.6 ml), piperidine (720 μl, 7.3 mmol) was added and the mixture was stirred under argon at rt. The mixture was evaporated and purified by preparative HPLC (C18, acetonitrile/water with 0.1% formic acid) to give 232 mg (92% yield) of the target compound.

LC-MS (Method 1): Rt=1.24 min; MS (ESIpos): m/z=692 [M+H]⁺

Intermediate 35

### tri-tert-butyl (3S,10S,14S)-3-[(4-tert-butylphenyl)methyl]-1-{(1r,4S)-4-[({[(9H-fluoren-9-yl)methoxy]carbonyl}amino)methyl]cyclohexyl}-1,4,12-trioxo-2,5,11,13-tetraazahexadecane-10,14,16-tricarboxylate

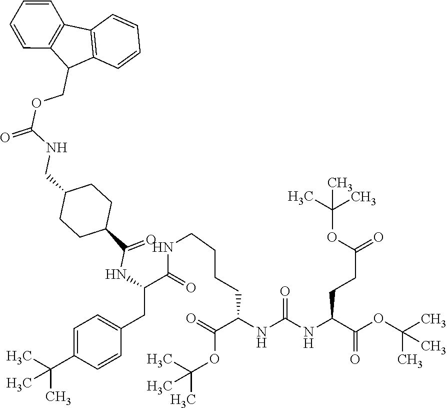 [SMILES: CC(C)(C)OC(=O)CC[C@H](NC(=O)N[C@@H](CCCCNC(=O)[C@H](Cc1ccc(C(C)(C)C)cc1)NC(=O)[C@H]1CC[C@H](CNC(=O)OCC2c3ccccc3-c3ccccc32)CC1)C(=O)OC(C)(C)C)C(=O)OC(C)(C)C]

di-tert-butyl (2S)-2-({[(2S)-6-{[(2S)-2-amino-3-(4-tert-butylphenyl)propanoyl]amino}-1-tert-butoxy-1-oxohexan-2-yl]carbamoyl}amino)pentanedioate (232 mg, 336 μmol) and (1r,4r)-4-[({[(9H-fluoren-9-yl)methoxy]carbonyl}amino)methyl]cyclohexane-1-carboxylic acid (191 mg, 504 μmol) were solubilised in DMF (2.6 ml), 4-methylmorpholine (110 μl, 1.0 mmol, CAS-RN: 109-02-4) and HATU (192 mg, 504 μmol) were added and the mixture was stirred under argon at rt. The mixture was evaporated and purified by preparative HPLC (C18, acetonitrile/water with 0.1% formic acid) to give 200 mg (57% yield) of the target compound.

LC-MS (Method 1): Rₜ=1.72 min; MS (ESIpos): m/z=1053 [M+H]⁺

¹H-NMR (400 MHz, DMSO-d6) δ[ppm]: 1.230 (9.12), 1.380 (16.00), 1.385 (14.22), 1.387 (15.49), 2.298 (0.69), 2.326 (0.41), 2.518 (1.55), 2.522 (1.02), 2.668 (0.46), 2.727 (1.20), 2.729 (1.29), 2.888 (1.44), 4.283 (0.69), 4.300 (0.46), 7.098 (0.51), 7.119 (0.71), 7.236 (0.82), 7.257 (0.77), 7.313 (0.80), 7.316 (0.77), 7.332 (0.53), 7.335 (0.49), 7.389 (0.42), 7.408 (0.65), 7.672 (0.62), 7.691 (0.55), 7.878 (0.80), 7.896 (0.65).

Intermediate 36

### tri-tert-butyl (3S,10S,14S)-1-[(1r,4S)-4-(aminomethyl)cyclohexyl]-3-[(4-tert-butylphenyl)methyl]-1,4,12-trioxo-2,5,11,13-tetraazahexadecane-10,14,16-tricarboxylate

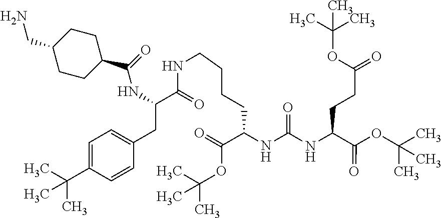 [SMILES: CC(C)(C)OC(=O)CC[C@H](NC(=O)N[C@@H](CCCCNC(=O)[C@H](Cc1ccc(C(C)(C)C)cc1)NC(=O)[C@H]1CC[C@H](CN)CC1)C(=O)OC(C)(C)C)C(=O)OC(C)(C)C]

tri-tert-butyl (3S,10S,14S)-3-[(4-tert-butylphenyl)methyl]-1-{(1r,4S)-4-[({[(9H-fluoren-9-yl)methoxy]carbonyl}amino)methyl]cyclohexyl}-1,4,12-trioxo-2,5,11,13-tetraazahexadecane-10,14,16-tricarboxylate (200 mg, 190 μmol) was solubilised in DMF (5.0 ml), piperidine (380 μl, 3.8 mmol) was added and the mixture was stirred under argon at rt. The mixture was evaporated and purified by preparative HPLC (C18, acetonitrile/water with 0.1% formic acid) to give 116 mg (74% yield) of the target compound.

LC-MS (Method 1): Rt=1.25 min; MS (ESIpos): m/z=831 [M+H]⁺

¹H-NMR (400 MHz, DMSO-d6) δ[ppm]: 1.235 (12.06), 1.383 (16.00), 1.387 (14.27), 1.389 (14.85), 2.518 (0.83), 2.522 (0.58), 2.530 (0.61), 2.548 (0.56), 7.097 (0.66), 7.117 (0.87), 7.233 (1.03), 7.254 (0.74), 8.409 (1.16).

### Example 8-A

### N⁶—{N-[(1r,4S)-4-(aminomethyl)cyclohexane-1-carbonyl]-4-tert-butyl-L-phenylalanyl}-N²-{[(1S)-1,3-dicarboxypropyl]carbamoyl}-L-lysine

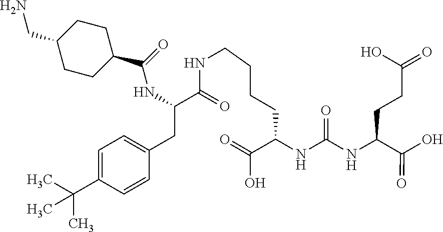 [SMILES: CC(C)(C)c1ccc(C[C@H](NC(=O)[C@H]2CC[C@H](CN)CC2)C(=O)NCCCC[C@H](NC(=O)N[C@@H](CCC(=O)O)C(=O)O)C(=O)O)cc1]

tri-tert-butyl (3S,10S,14S)-1-[(1r,4S)-4-(aminomethyl)cyclohexyl]-3-[(4-tert-butylphenyl)methyl]-1,4,12-trioxo-2,5,11,13-tetraazahexadecane-10,14,16-tricarboxylate (21.0 mg, 98% purity, 24.8 μmol) was solubilised in DCM (1.5 ml), TFA (1.5 ml, 20 mmol) was added and the mixture was stirred under argon at rt. The mixture was evaporated and purified by preparative HPLC (C18, acetonitrile/water with 0.1% formic acid) to give 5.00 mg (90% purity, 27% yield) of the target compound.

LC-MS (Method 1): Rₜ=0.83 min; MS (ESIpos): m/z=663 [M+H]⁺

¹H-NMR (400 MHz, DMSO-d6) δ[ppm]: 1.236 (16.00), 2.331 (0.42), 2.518 (2.09), 2.522 (1.32), 2.539 (0.49), 2.673 (0.44), 3.350 (0.90), 7.105 (0.86), 7.125 (1.12), 7.236 (1.34), 7.257 (0.93).

#9 Linker

Intermediate 37

### tri-tert-butyl (5S,12S,16S)-1-(9H-fluoren-9-yl)-3,6,14-trioxo-5-{4-[(pyridine-3-carbonyl)amino]butyl}-2-oxa-4,7,13,15-tetraazaoctadecane-12,16,18-tricarboxylate

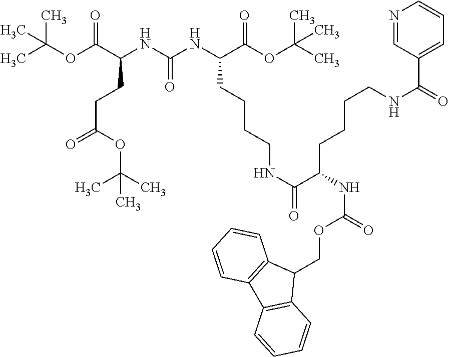 [SMILES: CC(C)(C)OC(=O)CC[C@H](NC(=O)N[C@@H](CCCCNC(=O)[C@H](CCCCNC(=O)c1cccnc1)NC(=O)OCC1c2ccccc2-c2ccccc21)C(=O)OC(C)(C)C)C(=O)OC(C)(C)C]

di-tert-butyl N-{[(2S)-6-amino-1-tert-butoxy-1-oxohexan-2-yl]carbamoyl}-L-glutamate (300 mg, 70% purity, 431 μmol) and N2-{[(9H-fluoren-9-yl)methoxy]carbonyl}-N6-(pyridine-3-carbonyl)-L-lysine (306 mg, 646 μmol) were solubilised in DMF (3.3 ml), 4-methylmorpholine (140 μl, 1.3 mmol, CAS-RN: 109-02-4) and HATU (246 mg, 646 μmol) were added and the mixture was stirred under argon at rt. The mixture was evaporated and purified by preparative HPLC (C18, acetonitrile/water with 0.1% formic acid) to give 280 mg (98% purity, 68% yield) of the target compound.

LC-MS (Method 1): Rₜ=1.47 min; MS (ESIpos): m/z=944 [M+H]⁺

¹H-NMR (400 MHz, DMSO-d6) δ[ppm]: 1.377 (16.00), 2.297 (0.49), 2.522 (0.45), 4.204 (0.54), 7.310 (0.49), 7.409 (0.49), 7.877 (0.68), 7.896 (0.56).

Intermediate 38

### tri-tert-butyl (7S,14S,18S)-7-amino-1,8,16-trioxo-1-(pyridin-3-yl)-2,9,15,17-tetraazaicosane-14,18,20-tricarboxylate

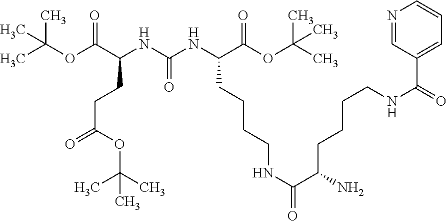 [SMILES: CC(C)(C)OC(=O)CC[C@H](NC(=O)N[C@@H](CCCCNC(=O)[C@@H](N)CCCCNC(=O)c1cccnc1)C(=O)OC(C)(C)C)C(=O)OC(C)(C)C]

tri-tert-butyl (5S,12S,16S)-1-(9H-fluoren-9-yl)-3,6,14-trioxo-5-{4-[(pyridine-3-carbonyl)amino]butyl}-2-oxa-4,7,13,15-tetraazaoctadecane-12,16,18-tricarboxylate (280 mg, 297 μmol) was solubilised in DMF (7.8 ml), piperidine (590 μl, 5.9 mmol) was added and the mixture was stirred under argon at rt. The mixture was evaporated and purified by preparative HPLC (C18, acetonitrile/water with 0.2% ammonia) to give 111 mg (98% purity, 51% yield) of the target compound.

LC-MS (Method 2): Rₜ=1.22 min; MS (ESIpos): m/z=722 [M+H]⁺

¹H-NMR (400 MHz, DMSO-d6) δ[ppm]: 1.371 (1.15), 1.382 (16.00), 1.386 (13.46), 1.390 (12.62), 2.297 (0.53), 2.518 (1.35), 2.522 (0.84), 3.248 (0.68), 3.263 (0.64), 8.253 (1.43), 8.680 (0.48), 8.684 (0.49), 8.692 (0.49), 8.696 (0.44), 8.980 (0.53), 8.982 (0.60), 8.986 (0.58), 8.988 (0.55).

Intermediate 39

### tri-tert-butyl (7S,14S,18S)-7-({(1r,4S)-4-[({[(9H-fluoren-9-yl)methoxy]carbonyl}amino)methyl]cyclohexane-1-carbonyl}amino)-1,8,16-trioxo-1-(pyridin-3-yl)-2,9,15,17-tetraazaicosane-14,18,20-tricarboxylate

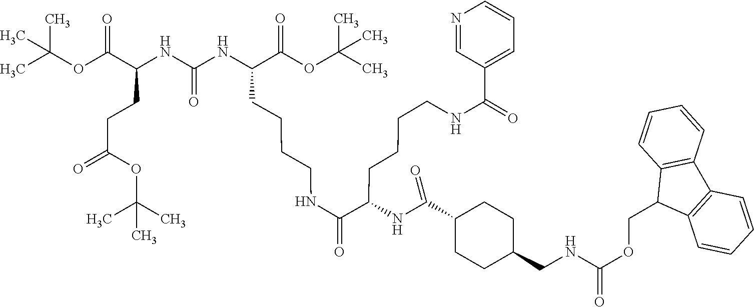 [SMILES: CC(C)(C)OC(=O)CC[C@H](NC(=O)N[C@@H](CCCCNC(=O)[C@H](CCCCNC(=O)c1cccnc1)NC(=O)[C@H]1CC[C@H](CNC(=O)OCC2c3ccccc3-c3ccccc32)CC1)C(=O)OC(C)(C)C)C(=O)OC(C)(C)C]

tri-tert-butyl (7S,14S,18S)-7-amino-1,8,16-trioxo-1-(pyridin-3-yl)-2,9,15,17-tetraazaicosane-14,18,20-tricarboxylate (111 mg, 154 μmol) and (1r,4r)-4-[({[(9H-fluoren-9-yl)methoxy]carbonyl}amino)methyl]cyclohexane-1-carboxylic acid (87.6 mg, 231 μmol) were solubilised in DMF (1.2 ml), 4-methylmorpholine (51 μl, 460 μmol, CAS-RN: 109-02-4) and HATU (87.8 mg, 231 μmol) were added and the mixture was stirred under argon at rt. The mixture was evaporated and purified by preparative HPLC (C18, acetonitrile/water with 0.1% formic acid) to give 119 mg (98% purity, 70% yield) of the target compound.

LC-MS (Method 1): Rₜ=1.48 min; MS (ESIpos): m/z=1083 [M+H]⁺

¹H-NMR (400 MHz, DMSO-d6) δ[ppm]: 1.239 (0.46), 1.378 (16.00), 1.386 (9.24), 2.297 (0.52), 2.518 (0.83), 2.522 (0.51), 4.293 (0.57), 7.320 (0.67), 7.322 (0.68), 7.338 (0.45), 7.341 (0.44), 7.410 (0.60), 7.684 (0.53), 7.702 (0.49), 7.879 (0.67), 7.898 (0.61), 8.978 (0.43), 8.982 (0.43).

Intermediate 40

### formic acid-tri-tert-butyl (7S,14S,18S)-7-{[(1r,4S)-4-(aminomethyl)cyclohexane-1-carbonyl]amino}-1,8,16-trioxo-1-(pyridin-3-yl)-2,9,15,17-tetraazaicosane-14,18,20-tricarboxylate (1/1)

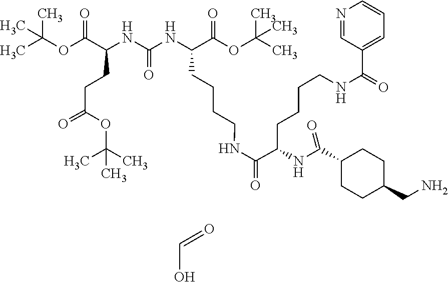 [SMILES: CC(C)(C)OC(=O)CC[C@H](NC(=O)N[C@@H](CCCCNC(=O)[C@H](CCCCNC(=O)c1cccnc1)NC(=O)[C@H]1CC[C@H](CN)CC1)C(=O)OC(C)(C)C)C(=O)OC(C)(C)C ; O=CO]

tri-tert-butyl (7S,14S,18S)-7-({(1r,4S)-4-[({[(9H-fluoren-9-yl)methoxy]carbonyl}amino)methyl]cyclohexane-1-carbonyl}amino)-1,8,16-trioxo-1-(pyridin-3-yl)-2,9,15,17-tetraazaicosane-14,18,20-tricarboxylate (119 mg, 98% purity, 108 μmol) was solubilised in DMF (2.8 ml), piperidine (210 μl, 2.2 mmol) was added and the mixture was stirred under argon for 3 h at rt. The mixture was evaporated and purified by preparative HPLC (C18, acetonitrile/water with 0.1% formic acid) to give 44.0 mg (98% purity, 44% yield) of the target compound.

LC-MS (Method 1): Rₜ=1.05 min; MS (ESIpos): m/z=861 [M+H]⁺

¹H-NMR (400 MHz, DMSO-d6) δ[ppm]: 1.240 (0.42), 1.382 (16.00), 1.389 (8.48), 2.518 (1.89), 2.523 (1.38), 3.231 (0.43), 3.250 (0.50), 8.418 (0.94), 8.979 (0.40), 8.983 (0.41).

### Example 9-A

N⁶—{N²-[(1r,4S)-4-(aminomethyl)cyclohexane-1-carbonyl]-N⁶-(pyridine-3-carbonyl)-L-lysyl}-N²-{[(1S)-1,3-dicarboxypropyl]carbamoyl}-L-lysine

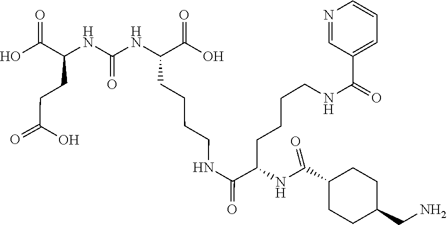 [SMILES: NC[C@H]1CC[C@H](C(=O)N[C@@H](CCCCNC(=O)c2cccnc2)C(=O)NCCCC[C@H](NC(=O)N[C@@H](CCC(=O)O)C(=O)O)C(=O)O)CC1]

formic acid-tri-tert-butyl (7S,14S,18S)-7-{[(1r,4S)-4-(aminomethyl)cyclohexane-1-carbonyl]amino}-1,8,16-trioxo-1-(pyridin-3-yl)-2,9,15,17-tetraazaicosane-14,18,20-tricarboxylate (1/1) (13.4 mg, 98% purity, 14.5 μmol) was solubilised in DCM (1.0 ml), TFA (1.0 ml, 13 mmol) was added and the mixture was stirred under argon at rt. The mixture was evaporated and purified by preparative HPLC (C18, acetonitrile/water with 0.1% formic acid) to give 8.00 mg (95% purity, 76% yield) of the target compound.

LC-MS (Method 1): Rₜ=0.49 min; MS (ESIpos): m/z=693 [M+H]⁺

¹H-NMR (400 MHz, DMSO-d6) δ[ppm]: 0.858 (1.30), 0.888 (3.12), 0.916 (3.45), 0.944 (1.50), 1.143 (0.46), 1.253 (7.80), 1.314 (4.55), 1.330 (5.40), 1.349 (4.94), 1.427 (2.73), 1.446 (3.77), 1.463 (4.68), 1.478 (5.27), 1.496 (6.18), 1.512 (5.66), 1.533 (4.10), 1.548 (2.93), 1.571 (2.60), 1.602 (3.06), 1.623 (3.51), 1.636 (3.71), 1.657 (5.20), 1.670 (5.85), 1.702 (5.33), 1.750 (3.64), 1.767 (3.51), 1.787 (3.84), 1.820 (1.56), 1.839 (0.72), 2.073 (0.46), 2.107 (1.63), 2.137 (3.32), 2.164 (3.51), 2.177 (3.06), 2.197 (2.08), 2.204 (2.21), 2.226 (3.51), 2.247 (2.02), 2.262 (1.82), 2.284 (0.91), 2.322 (3.06), 2.326 (4.03), 2.331 (2.99), 2.517 (16.00), 2.522 (10.08), 2.539 (14.83), 2.627 (4.88), 2.664 (3.64), 2.668 (4.42), 2.673 (3.25), 2.949 (2.15), 2.965 (2.93), 2.983 (3.84), 2.997 (3.71), 3.022 (3.90), 3.038 (4.29), 3.055 (3.71), 3.072 (3.12), 3.219 (7.93), 3.236 (12.10), 3.252 (12.68), 3.268 (9.89), 3.385 (11.45), 3.944 (5.79), 4.108 (1.89), 4.129 (3.19), 4.143 (3.25), 4.164 (1.76), 6.151 (2.15), 6.166 (2.08), 6.390 (3.19), 6.410 (3.12), 7.471 (3.51), 7.483 (3.84), 7.490 (3.90), 7.502 (3.77), 7.766 (2.21), 7.780 (4.23), 7.792 (2.21), 7.874 (3.84), 7.894 (3.71), 8.182 (3.12), 8.187 (4.94), 8.191 (3.45), 8.202 (3.25), 8.207 (4.75), 8.211 (3.25), 8.232 (4.88), 8.677 (5.01), 8.686 (5.01), 8.764 (2.41), 8.777 (4.75), 8.791 (2.41), 8.995 (7.28), 8.998 (7.22).

#10 Linker

Intermediate 41

### tri-tert-butyl (5S,12S,16S)-1-(9H-fluoren-9-yl)-5-[(3-fluorophenyl)methyl]-3,6,14-trioxo-2-oxa-4,7,13,15-tetraazaoctadecane-12,16,18-tricarboxylate

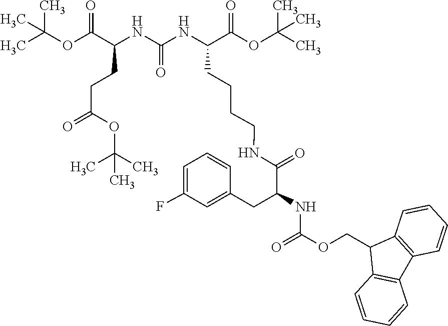 [SMILES: CC(C)(C)OC(=O)CC[C@H](NC(=O)N[C@@H](CCCCNC(=O)[C@H](Cc1cccc(F)c1)NC(=O)OCC1c2ccccc2-c2ccccc21)C(=O)OC(C)(C)C)C(=O)OC(C)(C)C]

di-tert-butyl N-{[(2S)-6-amino-1-tert-butoxy-1-oxohexan-2-yl]carbamoyl}-L-glutamate (290 mg, 70% purity, 416 μmol) and N-{[(9H-fluoren-9-yl)methoxy]carbonyl}-3-fluoro-L-phenylalanine (253 mg, 624 μmol) were solubilised in DMF (3.2 ml), 4-methylmorpholine (140 μl, 1.2 mmol, CAS-RN: 109-02-4) and HATU (237 mg, 624 μmol) were added and the mixture was stirred under argon at rt. The mixture was evaporated and purified by preparative HPLC (C18, acetonitrile/water with 0.1% formic acid) to give 310 mg (95% purity, 81% yield) of the target compound.

LC-MS (Method 1): Rₜ=1.63 min; MS (ESIpos): m/z=876 [M+H]⁺

¹H-NMR (400 MHz, DMSO-d6) δ[ppm]: 1.380 (16.00), 2.297 (1.42), 4.126 (0.41), 6.274 (0.44), 7.250 (0.42), 7.271 (0.42), 7.402 (0.41), 7.637 (0.56), 7.868 (0.61), 7.887 (0.55).

Intermediate 42

### di-tert-butyl (2S)-2-({[(2S)-6-{[(2S)-2-amino-3-(3-fluorophenyl)propanoyl]amino}-1-tert-butoxy-1-oxohexan-2-yl]carbamoyl}amino)pentanedioate

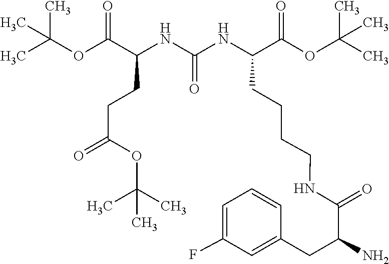 [SMILES: CC(C)(C)OC(=O)CC[C@H](NC(=O)N[C@@H](CCCCNC(=O)[C@@H](N)Cc1cccc(F)c1)C(=O)OC(C)(C)C)C(=O)OC(C)(C)C]

tri-tert-butyl (5S,12S,16S)-1-(9H-fluoren-9-yl)-5-[(3-fluorophenyl)methyl]-3,6,14-trioxo-2-oxa-4,7,13,15-tetraazaoctadecane-12,16,18-tricarboxylate (310 mg, 92% purity, 326 μmol) was solubilised in DMF (8.6 ml), piperidine (640 μl, 6.5 mmol) was added and the mixture was stirred under argon at rt. The mixture was evaporated and purified by preparative HPLC (C18, acetonitrile/water with 0.1% formic acid) to give 204 mg (96% yield) of the target compound.

LC-MS (Method 1): Rt=1.14 min; MS (ESIpos): m/z=654 [M+H]⁺

¹H-NMR (400 MHz, DMSO-d6) δ[ppm]: 1.384 (12.08), 1.392 (16.00), 2.327 (0.44), 2.518 (1.62), 2.523 (1.03), 2.665 (0.47), 2.668 (0.53), 7.010 (0.66), 7.013 (0.63), 7.032 (0.73), 8.194 (1.14).

Intermediate 43

### tri-tert-butyl (3S,10S,14S)-1-{(1r,4S)-4-[({[(9H-fluoren-9-yl)methoxy]carbonyl}amino)methyl]cyclohexyl}-3-[(3-fluorophenyl)methyl]-1,4,12-trioxo-2,5,11,13-tetraazahexadecane-10,14,16-tricarboxylate

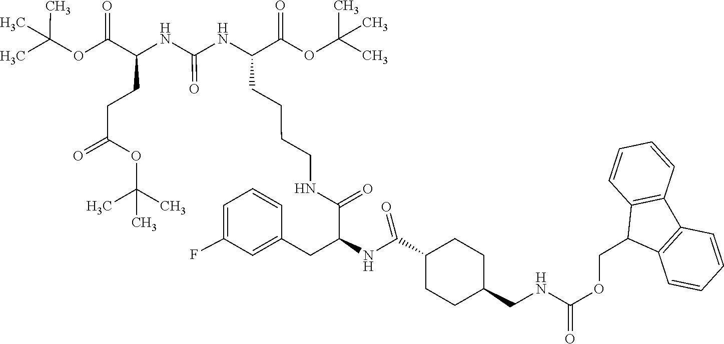 [SMILES: CC(C)(C)OC(=O)CC[C@H](NC(=O)N[C@@H](CCCCNC(=O)[C@H](Cc1cccc(F)c1)NC(=O)[C@H]1CC[C@H](CNC(=O)OCC2c3ccccc3-c3ccccc32)CC1)C(=O)OC(C)(C)C)C(=O)OC(C)(C)C]

di-tert-butyl (2S)-2-({[(2S)-6-{[(2S)-2-amino-3-(3-fluorophenyl)propanoyl]amino}-1-tert-butoxy-1-oxohexan-2-yl]carbamoyl}amino)pentanedioate (204 mg, 96% purity, 300 μmol) and (1r,4r)-4-[({[(9H-fluoren-9-yl)methoxy]carbonyl}amino)methyl]cyclohexane-1-carboxylic acid (171 mg, 450 μmol) were solubilised in DMF (2.3 ml), 4-methylmorpholine (99 μl, 900 μmol, CAS-RN: 109-02-4) and HATU (171 mg, 450 μmol) were added and the mixture was stirred under argon at rt.

The mixture was evaporated and purified by preparative HPLC (C18, acetonitrile/water with 0.1% formic acid) to give 80.0 mg (26% yield) of the target compound.

LC-MS (Method 1): Rt=1.62 min; MS (ESIpos): m/z=1015 [M+H]⁺

Intermediate 44

### tri-tert-butyl (3S,10S,14S)-1-[(1r,4S)-4-(aminomethyl)cyclohexyl]-3-[(3-fluorophenyl)methyl]-1,4,12-trioxo-2,5,11,13-tetraazahexadecane-10,14,16-tricarboxylate

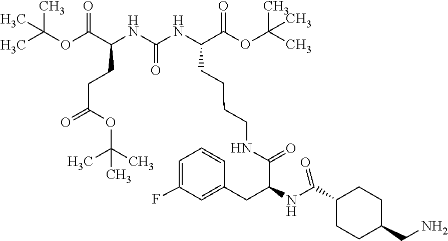 [SMILES: CC(C)(C)OC(=O)CC[C@H](NC(=O)N[C@@H](CCCCNC(=O)[C@H](Cc1cccc(F)c1)NC(=O)[C@H]1CC[C@H](CN)CC1)C(=O)OC(C)(C)C)C(=O)OC(C)(C)C]

tri-tert-butyl (3S,10S,14S)-1-{(1r,4S)-4-[({[(9H-fluoren-9-yl)methoxy]carbonyl}amino)methyl]cyclohexyl}-3-[(3-fluorophenyl)methyl]-1,4,12-trioxo-2,5,11,13-tetraazahexadecane-10,14,16-tricarboxylate (80.0 mg, 80% purity, 63.1 μmol) was solubilised in DMF (1.7 ml), piperidine (120 μl, 1.3 mmol) was added and the mixture was stirred under argon at rt. The mixture was evaporated and purified by preparative HPLC (C18, acetonitrile/water with 0.1% formic acid) to give 40.0 mg (80% yield) of the target compound.

LC-MS (Method 1): Rt=1.19 min; MS (ESIpos): m/z=793 [M+H]⁺

¹H-NMR (400 MHz, DMSO-d6) δ[ppm]: 1.383 (15.49), 1.389 (16.00), 2.518 (1.98), 2.522 (1.57), 7.048 (0.44), 8.417 (1.51).

### Example 10-A

### N⁶—{N-[(1r,4S)-4-(aminomethyl)cyclohexane-1-carbonyl]-3-fluoro-L-phenylalanyl}-N²-{[(1S)-1,3-dicarboxypropyl]carbamoyl}-L-lysine

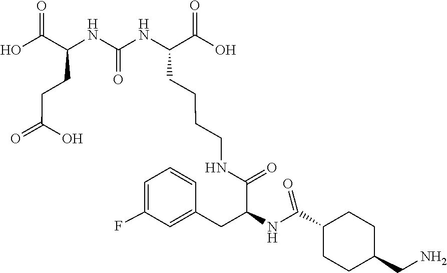 [SMILES: NC[C@H]1CC[C@H](C(=O)N[C@@H](Cc2cccc(F)c2)C(=O)NCCCC[C@H](NC(=O)N[C@@H](CCC(=O)O)C(=O)O)C(=O)O)CC1]

tri-tert-butyl (3S,10S,14S)-1-[(1r,4S)-4-(aminomethyl)cyclohexyl]-3-[(3-fluorophenyl)methyl]-1,4,12-trioxo-2,5,11,13-tetraazahexadecane-10,14,16-tricarboxylate (23.5 mg, 98% purity, 29.1 μmol) was solubilised in DCM (1.8 ml), TFA (1.8 ml, 23 mmol) was added and the mixture was stirred under argon at rt. The mixture was evaporated and purified by preparative HPLC (C18, acetonitrile/water with 0.1% formic acid) to give 11.0 mg (98% purity, 59% yield) of the target compound.

LC-MS (Method 1): Rt=0.63 min; MS (ESIpos): m/z=625 [M+H]⁺

¹H-NMR (400 MHz, DMSO-d6) δ[ppm]: 0.851 (0.48), 0.880 (1.18), 0.911 (1.25), 0.937 (0.62), 1.123 (0.69), 1.146 (0.83), 1.203 (0.90), 1.234 (1.73), 1.327 (1.45), 1.346 (1.59), 1.426 (1.11), 1.444 (1.18), 1.462 (0.90), 1.507 (0.83), 1.548 (0.90), 1.569 (1.18), 1.580 (1.32), 1.591 (1.18), 1.666 (1.25), 1.698 (1.11), 1.743 (0.76), 1.777 (0.90), 1.795 (0.62), 1.811 (0.62), 1.835 (0.69), 1.852 (0.48), 1.905 (1.11), 2.048 (0.55), 2.074 (4.23), 2.112 (0.69), 2.124 (0.62), 2.147 (0.90), 2.160 (0.90), 2.178 (0.55), 2.232 (0.62), 2.256 (0.90), 2.267 (0.62), 2.276 (0.69), 2.287 (0.55), 2.331 (2.98), 2.336 (1.39), 2.518 (15.38), 2.522 (9.77), 2.539 (1.39), 2.594 (2.42), 2.611 (2.49), 2.673 (2.98), 2.756 (0.83), 2.782 (0.97), 2.791 (1.18), 2.816 (1.04), 2.916 (0.62), 2.929 (0.83), 2.949 (1.87), 2.962 (1.94), 2.983 (1.11), 2.996 (0.97), 3.086 (1.04), 3.102 (1.32), 3.119 (1.32), 3.135 (1.11), 3.336 (16.00), 3.932 (1.59), 3.943 (1.73), 4.389 (0.55), 4.402 (0.62), 4.413 (0.90), 4.426 (0.97), 4.436 (0.62), 4.449 (0.48), 6.121 (0.62), 6.394 (1.11), 6.413 (1.11), 6.972 (0.76), 6.994 (1.52), 7.017 (0.97), 7.026 (1.45), 7.040 (2.01), 7.058 (3.05), 7.253 (0.97), 7.269 (1.18), 7.274 (1.45), 7.289 (1.59), 7.309 (0.69), 7.883 (0.83), 7.896 (1.39), 7.911 (0.69), 8.101 (1.39), 8.123 (1.32), 8.244 (0.97).

#11 Linker

Intermediate 45

### tri-tert-butyl (5S,12S,16S)-5-(4-acetamidobutyl)-1-(9H-fluoren-9-yl)-3,6,14-trioxo-2-oxa-4,7,13,15-tetraazaoctadecane-12,16,18-tricarboxylate

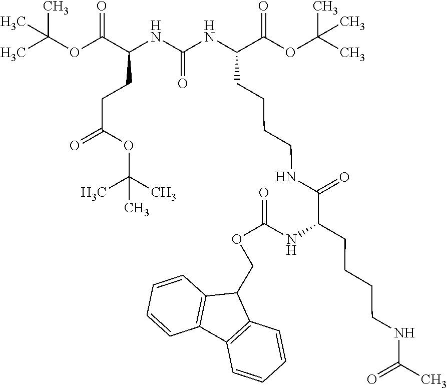 [SMILES: CC(=O)NCCCC[C@H](NC(=O)OCC1c2ccccc2-c2ccccc21)C(=O)NCCCC[C@H](NC(=O)N[C@@H](CCC(=O)OC(C)(C)C)C(=O)OC(C)(C)C)C(=O)OC(C)(C)C]

di-tert-butyl N-{[(2S)-6-amino-1-tert-butoxy-1-oxohexan-2-yl]carbamoyl}-L-glutamate (300 mg, 70% purity, 431 μmol) and N6-acetyl-N2-{[(9H-fluoren-9-yl)methoxy]carbonyl}-L-lysine (265 mg, 646 μmol) were solubilised in DMF (3.3 ml), 4-methylmorpholine (140 μl, 1.3 mmol, CAS-RN: 109-02-4) and HATU (246 mg, 646 μmol) were added and the mixture was stirred under argon at rt. The mixture was evaporated and purified by preparative HPLC (C18, acetonitrile/water with 0.1% formic acid) to give 255 mg (80% purity, 54% yield) of the target compound.

LC-MS (Method 1): Rₜ=1.47 min; MS (ESIpos): m/z=881 [M+H]⁺

Intermediate 46

### tri-tert-butyl (8S,15S,19S)-8-amino-2,9,17-trioxo-3,10,16,18-tetraazahenicosane-15,19,21-tricarboxylate

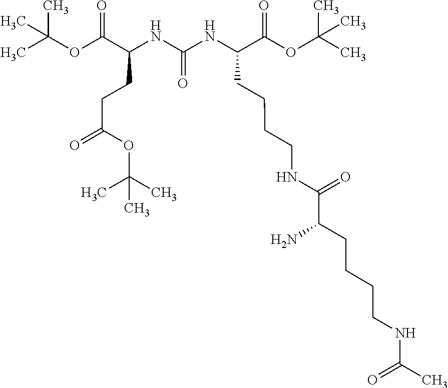 [SMILES: CC(=O)NCCCC[C@H](N)C(=O)NCCCC[C@H](NC(=O)N[C@@H](CCC(=O)OC(C)(C)C)C(=O)OC(C)(C)C)C(=O)OC(C)(C)C]

tri-tert-butyl (5S,12S,16S)-5-(4-acetamidobutyl)-1-(9H-fluoren-9-yl)-3,6,14-trioxo-2-oxa-4,7,13,15-tetraazaoctadecane-12,16,18-tricarboxylate (255 mg, 80% purity, 232 μmol) was solubilised in DMF (2.5 ml), piperidine (460 μl, 4.6 mmol) was added and the mixture was stirred under argon overnight at rt. The mixture was evaporated and purified by preparative HPLC (C18, acetonitrile/water with 0.1% formic acid) to give 71.0 mg (98% purity, 46% yield)) of the target compound.

LC-MS (Method 1): Rₜ=1.06 min; MS (ESIpos): m/z=659 [M+H]⁺

Intermediate 47

### tri-tert-butyl (8S,15S,19S)-8-({(1r,4S)-4-[({[(9H-fluoren-9-yl)methoxy]carbonyl}amino)methyl]cyclohexane-1-carbonyl}amino)-2,9,17-trioxo-3,10,16,18-tetraazahenicosane-15,19,21-tricarboxylate

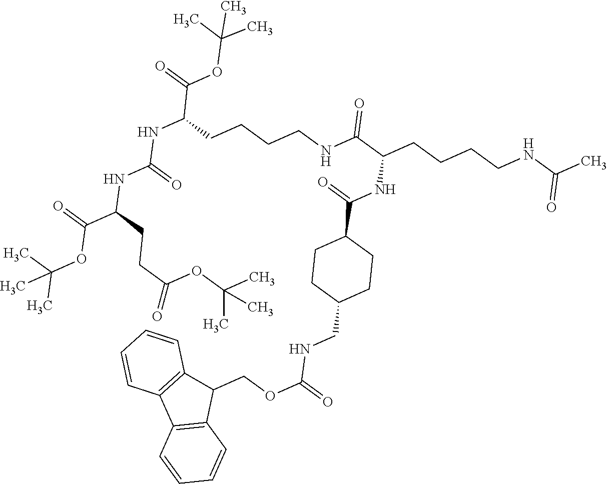 [SMILES: CC(=O)NCCCC[C@H](NC(=O)[C@H]1CC[C@H](CNC(=O)OCC2c3ccccc3-c3ccccc32)CC1)C(=O)NCCCC[C@H](NC(=O)N[C@@H](CCC(=O)OC(C)(C)C)C(=O)OC(C)(C)C)C(=O)OC(C)(C)C]

tri-tert-butyl (8S,15S,19S)-8-amino-2,9,17-trioxo-3,10,16,18-tetraazahenicosane-15,19,21-tricarboxylate (71.0 mg, 98% purity, 106 μmol) and (1r,4r)-4-[({[(9H-fluoren-9-yl)methoxy]carbonyl}amino)methyl]cyclohexane-1-carboxylic acid (60.2 mg, 159 μmol) were solubilised in DMF (810 μl), 4-methylmorpholine (35 μl, 320 μmol, CAS-RN: 109-02-4) and HATU (60.3 mg, 159 μmol) were added and the mixture was stirred under argon at rt. The mixture was evaporated and purified by preparative HPLC (C18, acetonitrile/water with 0.1% formic acid) to give 61.0 mg (85% purity, 48% yield) of the target compound.

LC-MS (Method 1): Rₜ=1.48 min; MS (ESIpos): m/z=1020 [M+H]⁺

Intermediate 48

### tri-tert-butyl (8S,15S,19S)-8-{[(1r,4S)-4-(aminomethyl)cyclohexane-1-carbonyl]amino}-2,9,17-trioxo-3,10,16,18-tetraazahenicosane-15,19,21-tricarboxylate

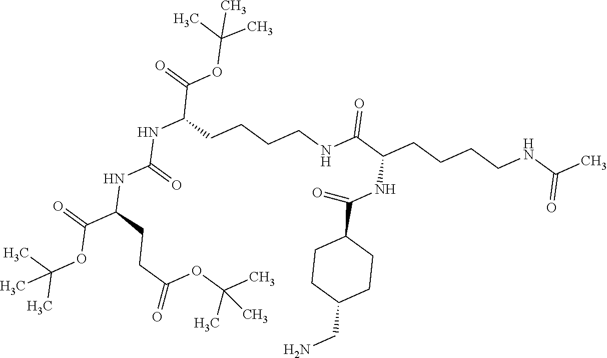 [SMILES: CC(=O)NCCCC[C@H](NC(=O)[C@H]1CC[C@H](CN)CC1)C(=O)NCCCC[C@H](NC(=O)N[C@@H](CCC(=O)OC(C)(C)C)C(=O)OC(C)(C)C)C(=O)OC(C)(C)C]

tri-tert-butyl (8S,15S,19S)-8-({(1r,4S)-4-[({[(9H-fluoren-9-yl)methoxy]carbonyl}amino)methyl]cyclohexane-1-carbonyl}amino)-2,9,17-trioxo-3,10,16,18-tetraazahenicosane-15,19,21-tricarboxylate (61.0 mg, 85% purity, 50.9 μmol) was solubilised in DMF (1.3 ml), piperidine (100 μl, 1.0 mmol) was added and the mixture was stirred under argon at rt. The mixture was evaporated and purified by preparative HPLC (C18, acetonitrile/water with 0.1% formic acid) to give 79.0 mg (73% purity) of the target compound.

LC-MS (Method 1): Rₜ=0.92 min; MS (ESIpos): m/z=798 [M+H]⁺

### Example 11-A

### N⁶—{N⁶-acetyl-N²-[(1r,4S)-4-(aminomethyl)cyclohexane-1-carbonyl]-L-lysyl}-N²-{[(1S)-1,3-dicarboxypropyl]carbamoyl}-L-lysine

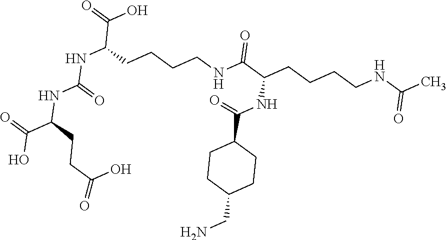 [SMILES: CC(=O)NCCCC[C@H](NC(=O)[C@H]1CC[C@H](CN)CC1)C(=O)NCCCC[C@H](NC(=O)N[C@@H](CCC(=O)O)C(=O)O)C(=O)O]

tri-tert-butyl (8S,15S,19S)-8-{[(1r,4S)-4-(aminomethyl)cyclohexane-1-carbonyl]amino}-2,9,17-trioxo-3,10,16,18-tetraazahenicosane-15,19,21-tricarboxylate (15.5 mg, 73% purity, 14.2 μmol) was solubilised in DCM (870 μl), TFA (870 μl, 11 mmol) was added and the mixture was stirred under argon at rt. The mixture was evaporated and purified by preparative HPLC (C18, acetonitrile/water with 0.1% formic acid) to give 11.0 mg (89% purity) of the target compound.

LC-MS (Method 1): Rₜ=0.47 min; MS (ESIpos): m/z=630 [M+H]⁺

¹H-NMR (400 MHz, DMSO-d6) δ[ppm]: 0.910 (0.76), 0.941 (0.86), 1.183 (0.55), 1.235 (1.01), 1.255 (1.47), 1.276 (1.49), 1.285 (1.37), 1.318 (1.53), 1.327 (1.39), 1.335 (1.43), 1.352 (1.88), 1.366 (1.40), 1.446 (0.63), 1.457 (0.72), 1.468 (1.03), 1.485 (1.02), 1.503 (0.95), 1.522 (0.67), 1.541 (0.53), 1.561 (0.46), 1.608 (0.53), 1.622 (0.57), 1.641 (0.45), 1.656 (0.40), 1.677 (0.43), 1.692 (0.50), 1.714 (0.93), 1.727 (1.31), 1.768 (16.00), 1.784 (1.15), 1.905 (0.50), 1.922 (0.57), 2.161 (0.68), 2.183 (0.43), 2.190 (0.43), 2.209 (0.75), 2.226 (1.15), 2.245 (1.27), 2.267 (0.66), 2.327 (0.41), 2.518 (1.93), 2.523 (1.20), 2.539 (2.24), 2.642 (1.01), 2.657 (1.48), 2.669 (1.24), 2.673 (1.25), 2.946 (0.78), 2.962 (1.88), 2.978 (2.09), 2.994 (1.29), 3.010 (0.78), 3.024 (0.69), 3.041 (0.44), 4.009 (0.49), 4.016 (0.76), 4.029 (0.77), 4.064 (0.43), 4.078 (0.53), 4.085 (0.87), 4.098 (0.92), 4.106 (0.81), 4.119 (0.81), 4.128 (0.68), 4.141 (0.69), 6.281 (1.27), 6.302 (1.26), 6.316 (1.40), 6.336 (1.29), 7.682 (1.36), 7.757 (1.29), 7.777 (1.60), 7.787 (1.27), 7.801 (0.62), 7.832 (0.60), 7.845 (1.18), 7.859 (0.58).

#11a Linker

Intermediate 49

### tri-tert-butyl (5S,12S,16S)-5-[([1,1′-biphenyl]-4-yl)methyl]-1-(9H-fluoren-9-yl)-3,6,14-trioxo-2-oxa-4,7,13,15-tetraazaoctadecane-12,16,18-tricarboxylate

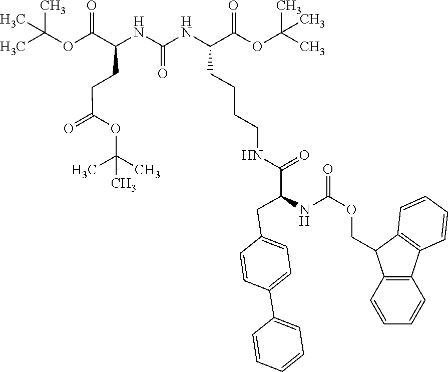 [SMILES: CC(C)(C)OC(=O)CC[C@H](NC(=O)N[C@@H](CCCCNC(=O)[C@H](Cc1ccc(-c2ccccc2)cc1)NC(=O)OCC1c2ccccc2-c2ccccc21)C(=O)OC(C)(C)C)C(=O)OC(C)(C)C]

di-tert-butyl N-{[(2S)-6-amino-1-tert-butoxy-1-oxohexan-2-yl]carbamoyl}-L-glutamate (280 mg, 70% purity, 402 μmol) and (2S)-3-([1,1′-biphenyl]-4-yl)-2-({[(9H-fluoren-9-yl)methoxy]carbonyl}amino)propanoic acid (279 mg, 603 μmol) were solubilised in DMF (3.1 ml), 4-methylmorpholine (130 μl, 1.2 mmol, CAS-RN: 109-02-4) and HATU (229 mg, 603 μmol) were added and the mixture was stirred under argon at rt. The mixture was evaporated and purified by preparative HPLC (C18, acetonitrile/water with 0.1% formic acid) to give 380 mg (95% purity, 96% yield) of the target compound.

LC-MS (Method 1): Rₜ=1.71 min; MS (ESIpos): m/z=934 [M+H]⁺

¹H-NMR (400 MHz, DMSO-d6) δ[ppm]: 1.376 (16.00), 2.223 (0.81), 2.997 (0.65), 4.137 (1.37), 6.277 (0.75), 7.377 (1.89), 7.394 (1.87), 7.582 (1.25), 7.651 (1.28), 7.880 (0.92).

Intermediate 50

### di-tert-butyl (2S)-2-({[(2S)-6-{[(2S)-2-amino-3-([1,1′-biphenyl]-4-yl)propanoyl]amino}-1-tert-butoxy-1-oxohexan-2-yl]carbamoyl}amino)pentanedioate

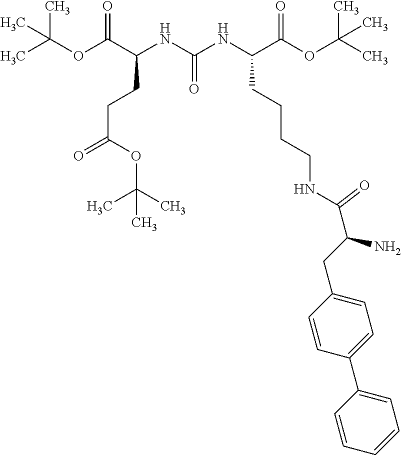 [SMILES: CC(C)(C)OC(=O)CC[C@H](NC(=O)N[C@@H](CCCCNC(=O)[C@@H](N)Cc1ccc(-c2ccccc2)cc1)C(=O)OC(C)(C)C)C(=O)OC(C)(C)C]

tri-tert-butyl (5S,12S,16S)-5-[([1,1′-biphenyl]-4-yl)methyl]-1-(9H-fluoren-9-yl)-3,6,14-trioxo-2-oxa-4,7,13,15-tetraazaoctadecane-12,16,18-tricarboxylate (310 mg, 92% purity, 306 μmol) was solubilised in DMF (8.0 ml), piperidine (600 μl, 6.1 mmol) was added and the mixture was stirred under argon at rt. The mixture was evaporated and purified by preparative HPLC (C18, acetonitrile/water with 0.1% formic acid) to give 254 mg (82% purity, 96% yield) of the target compound.

LC-MS (Method 1): Rt=1.26 min; MS (ESIpos): m/z=712 [M+H]⁺

Intermediate 51

### tri-tert-butyl (3S,10S,14S)-3-[([1,1′-biphenyl]-4-yl)methyl]-1-{(1r,4S)-4-[({[(9H-fluoren-9-yl)methoxy]carbonyl}amino)methyl]cyclohexyl}-1,4,12-trioxo-2,5,11,13-tetraazahexadecane-10,14,16-tricarboxylate

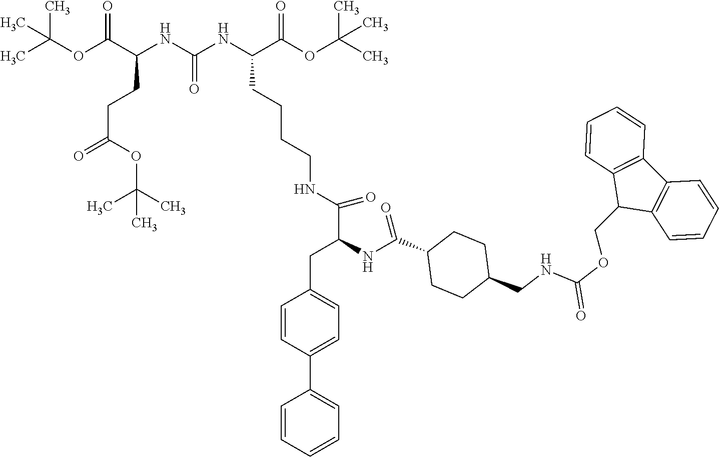 [SMILES: CC(C)(C)OC(=O)CC[C@H](NC(=O)N[C@@H](CCCCNC(=O)[C@H](Cc1ccc(-c2ccccc2)cc1)NC(=O)[C@H]1CC[C@H](CNC(=O)OCC2c3ccccc3-c3ccccc32)CC1)C(=O)OC(C)(C)C)C(=O)OC(C)(C)C]

di-tert-butyl (2S)-2-({[(2S)-6-{[(2S)-2-amino-3-([1,1′-biphenyl]-4-yl)propanoyl]amino}-1-tert-butoxy-1-oxohexan-2-yl]carbamoyl}amino)pentanedioate (254 mg, 82% purity, 293 μmol) and (1 r,4r)-4-[({[(9H-fluoren-9-yl)methoxy]carbonyl}amino)methyl]cyclohexane-1-carboxylic acid (167 mg, 439 μmol) were solubilised in DMF (2.3 ml), 4-methylmorpholine (97 μl, 880 μmol, CAS-RN: 109-02-4) and HATU (167 mg, 439 μmol) were added and the mixture was stirred under argon overnight at rt. The mixture was evaporated and purified by preparative HPLC (C18, acetonitrile/water with 0.1% formic acid) to give 78.0 mg (25% yield) of the target compound.

LC-MS (Method 1): Rₜ=1.69 min; MS (ESIpos): m/z=1073 [M+H]⁺

¹H-NMR (400 MHz, DMSO-d6) δ[ppm]: 1.233 (0.82), 1.378 (16.00), 1.384 (12.12), 2.298 (0.51), 2.323 (0.61), 2.327 (0.81), 2.332 (0.58), 2.523 (2.52), 2.665 (0.63), 2.669 (0.82), 2.673 (0.60), 2.729 (1.20), 2.794 (0.53), 2.888 (1.32), 4.282 (0.67), 4.298 (0.49), 7.289 (0.86), 7.308 (1.34), 7.325 (0.65), 7.378 (0.40), 7.397 (0.64), 7.417 (0.72), 7.437 (0.75), 7.546 (0.65), 7.565 (0.58), 7.620 (0.67), 7.639 (0.54), 7.668 (0.65), 7.687 (0.58), 7.866 (0.65), 7.885 (0.64).

Intermediate 52

### formic acid-tri-tert-butyl (3S,10S,14S)-1-[(1r,4S)-4-(aminomethyl)cyclohexyl]-3-[([1,1′-biphenyl]-4-yl)methyl]-1,4,12-trioxo-2,5,11,13-tetraazahexadecane-10,14,16-tricarboxylate (1/1)

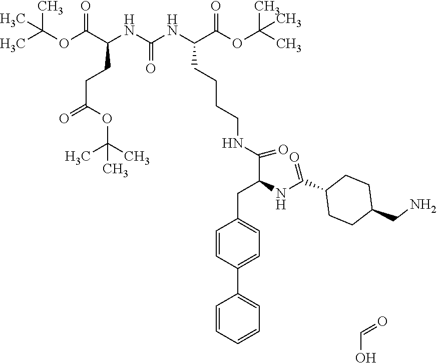 [SMILES: CC(C)(C)OC(=O)CC[C@H](NC(=O)N[C@@H](CCCCNC(=O)[C@H](Cc1ccc(-c2ccccc2)cc1)NC(=O)[C@H]1CC[C@H](CN)CC1)C(=O)OC(C)(C)C)C(=O)OC(C)(C)C ; O=CO]

tri-tert-butyl (3S,10S,14S)-3-[([1,1′-biphenyl]-4-yl)methyl]-1-{(1r,4S)-4-[({[(9H-fluoren-9-yl)methoxy]carbonyl}amino)methyl]cyclohexyl}-1,4,12-trioxo-2,5,11,13-tetraazahexadecane-10,14,16-tricarboxylate (78.0 mg, 75% purity, 54.6 μmol) was solubilised in DMF (1.4 ml), piperidine (110 μl, 1.1 mmol) was added and the mixture was stirred under argon overnight at rt. The mixture was evaporated and purified by preparative HPLC (C18, acetonitrile/water with 0.1% formic acid) to give 27.0 mg (98% purity, 54% yield) of the target compound.

LC-MS (Method 1): Rt=1.26 min; MS (ESIpos): m/z=851 [M+H]⁺

¹H-NMR (400 MHz, DMSO-d6) δ[ppm]: 1.381 (16.00), 1.386 (10.37), 2.518 (1.47), 2.522 (0.87), 7.285 (0.54), 7.306 (0.61), 7.441 (0.61), 7.540 (0.75), 7.561 (0.60), 7.617 (0.60), 7.635 (0.51), 8.424 (0.60).

### Example 11a-A

### (3S,10S,14S)-1-[(1r,4S)-4-(aminomethyl)cyclohexyl]-3-[([1,1′-biphenyl]-4-yl)methyl]-1,4,12-trioxo-2,5,11,13-tetraazahexadecane-10,14,16-tricarboxylic acid

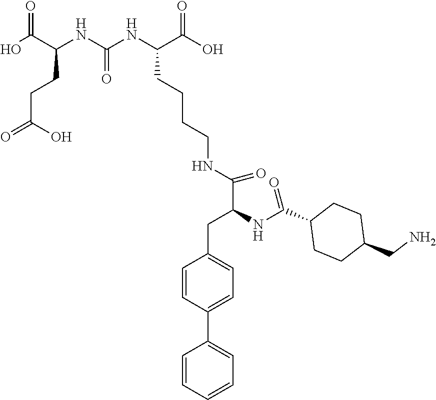 [SMILES: NC[C@H]1CC[C@H](C(=O)N[C@@H](Cc2ccc(-c3ccccc3)cc2)C(=O)NCCCC[C@H](NC(=O)N[C@@H](CCC(=O)O)C(=O)O)C(=O)O)CC1]

formic acid-tri-tert-butyl (3S,10S,14S)-1-[(1r,4S)-4-(aminomethyl)cyclohexyl]-3-[([1,1′-biphenyl]-4-yl)methyl]-1,4,12-trioxo-2,5,11,13-tetraazahexadecane-10,14,16-tricarboxylate (1/1) (11.4 mg, 98% purity, 12.5 μmol) was solubilised in DCM (770 μl), TFA (770 μl, 9.9 mmol) was added and the mixture was stirred under argon at rt. The mixture was evaporated and purified by preparative HPLC (C18, acetonitrile/water with 0.1% formic acid) to give 5.00 mg (98% purity, 58% yield) of the target compound.

LC-MS (Method 1): Rₜ=0.80 min; MS (ESIpos): m/z=683 [M+H]⁺

¹H-NMR (400 MHz, DMSO-d6) δ[ppm]: 0.851 (0.69), 0.883 (1.12), 0.913 (1.20), 0.936 (0.60), 1.160 (0.69), 1.184 (0.86), 1.215 (1.12), 1.237 (1.89), 1.258 (1.55), 1.339 (1.46), 1.352 (1.72), 1.437 (1.12), 1.570 (1.55), 1.693 (1.20), 1.752 (0.77), 1.783 (0.86), 1.816 (0.69), 1.841 (0.69), 1.905 (1.12), 2.068 (0.52), 2.097 (0.86), 2.128 (0.86), 2.151 (0.86), 2.167 (0.86), 2.234 (0.52), 2.254 (0.77), 2.278 (0.60), 2.289 (0.52), 2.332 (3.61), 2.336 (1.63), 2.518 (16.00), 2.523 (11.10), 2.586 (1.63), 2.604 (1.72), 2.673 (3.53), 2.678 (1.55), 2.790 (0.60), 2.813 (0.77), 2.824 (0.95), 2.848 (0.86), 2.925 (0.69), 2.938 (0.77), 2.957 (0.95), 2.972 (1.63), 2.986 (1.38), 3.007 (1.03), 3.020 (0.95), 3.106 (1.20), 3.122 (1.46), 3.139 (1.46), 3.154 (1.46), 3.940 (1.55), 4.412 (0.52), 4.434 (0.86), 4.447 (0.86), 4.470 (0.43), 6.123 (0.52), 6.400 (0.86), 6.420 (0.86), 7.293 (3.70), 7.314 (4.82), 7.333 (2.67), 7.352 (1.81), 7.421 (2.92), 7.441 (4.73), 7.459 (2.32), 7.541 (5.16), 7.561 (4.13), 7.621 (4.13), 7.639 (3.61), 7.642 (2.75), 7.888 (1.20), 8.114 (1.12), 8.135 (1.03), 8.262 (0.43).

#12 Linker

Intermediate 53

tri-tert-butyl (5S,12S,16S)-1-(9H-fluoren-9-yl)-5-[(2-methylphenyl)methyl]-3,6,14-trioxo-2-oxa-4,7,13,15-tetraazaoctadecane-12,16,18-tricarboxylate

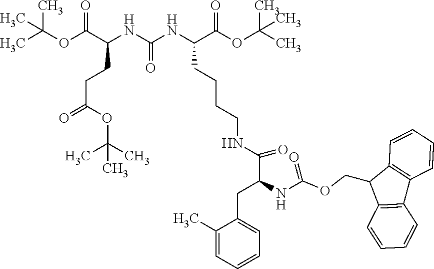 [SMILES: Cc1ccccc1C[C@H](NC(=O)OCC1c2ccccc2-c2ccccc21)C(=O)NCCCC[C@H](NC(=O)N[C@@H](CCC(=O)OC(C)(C)C)C(=O)OC(C)(C)C)C(=O)OC(C)(C)C]

di-tert-butyl N-{[(2S)-6-amino-1-tert-butoxy-1-oxohexan-2-yl]carbamoyl}-L-glutamate (300 mg, 70% purity, 431 μmol) and N-{[(9H-fluoren-9-yl)methoxy]carbonyl}-2-methyl-L-phenylalanine (259 mg, 646 μmol) were solubilised in DMF (3.3 ml), 4-methylmorpholine (140 μl, 1.3 mmol, CAS-RN: 109-02-4) and HATU (246 mg, 646 μmol) were added and the mixture was stirred under argon at rt. The mixture was evaporated and purified by preparative HPLC (C18, acetonitrile/water with 0.1% formic acid) to give 168 mg (92% purity, 41% yield) of the target compound.

LC-MS (Method 1): Rₜ=1.66 min; MS (ESIpos): m/z=872 [M+H]⁺

Intermediate 54

### di-tert-butyl (2S)-2-({[(2S)-6-{[(2S)-2-amino-3-(2-methylphenyl)propanoyl]amino}-1-tert-butoxy-1-oxohexan-2-yl]carbamoyl}amino)pentanedioate

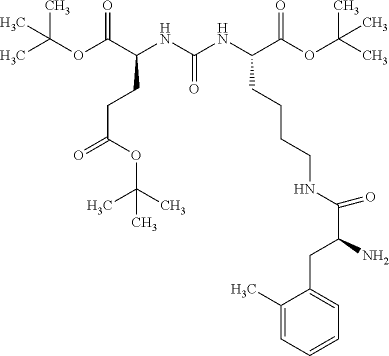 [SMILES: Cc1ccccc1C[C@H](N)C(=O)NCCCC[C@H](NC(=O)N[C@@H](CCC(=O)OC(C)(C)C)C(=O)OC(C)(C)C)C(=O)OC(C)(C)C]

tri-tert-butyl (5S,12S,16S)-1-(9H-fluoren-9-yl)-5-[(2-methylphenyl)methyl]-3,6,14-trioxo-2-oxa-4,7,13,15-tetraazaoctadecane-12,16,18-tricarboxylate (168 mg, 92% purity, 177 μmol) was solubilised in DMF (1.9 ml), piperidine (350 μl, 3.5 mmol) was added and the mixture was stirred under argon overnight at rt. The mixture was evaporated and purified by preparative HPLC (C18, acetonitrile/water with 0.1% formic acid) to give 45.0 mg (90% purity, 35% yield) of the target compound.

LC-MS (Method 1): Rₜ=1.12 min; MS (ESIpos): m/z=650 [M+H]⁺

Intermediate 55

### tri-tert-butyl (3S,10S,14S)-1-{(1r,4S)-4-[({[(9H-fluoren-9-yl)methoxy]carbonyl}amino)methyl]cyclohexyl}-3-[(2-methylphenyl)methyl]-1,4,12-trioxo-2,5,11,13-tetraazahexadecane-10,14,16-tricarboxylate

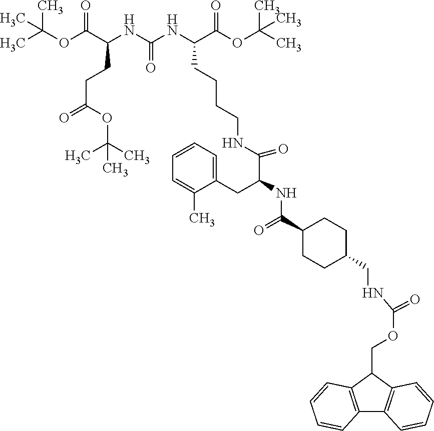 [SMILES: Cc1ccccc1C[C@H](NC(=O)[C@H]1CC[C@H](CNC(=O)OCC2c3ccccc3-c3ccccc32)CC1)C(=O)NCCCC[C@H](NC(=O)N[C@@H](CCC(=O)OC(C)(C)C)C(=O)OC(C)(C)C)C(=O)OC(C)(C)C]

di-tert-butyl (2S)-2-({[(2S)-6-{[(2S)-2-amino-3-(2-methylphenyl)propanoyl]amino}-1-tert-butoxy-1-oxohexan-2-yl]carbamoyl}amino)pentanedioate (45.0 mg, 90% purity, 62.4 μmol) and (1r,4r)-4-[({[(9H-fluoren-9-yl)methoxy]carbonyl}amino)methyl]cyclohexane-1-carboxylic acid (35.5 mg, 93.6 μmol) were solubilised in DMF (480 μl), 4-methylmorpholine (21 μl, 190 μmol, CAS-RN: 109-02-4) and HATU (35.6 mg, 93.6 μmol) were added and the mixture was stirred under argon overnight at rt. The mixture was evaporated and purified by preparative HPLC (C18, acetonitrile/water with 0.1% formic acid) to give 51.0 mg (93% purity, 75% yield) of the target compound.

LC-MS (Method 1): Rₜ=0.88 min; MS (ESIpos): m/z=1011 [M+H]⁺

Intermediate 56

### tri-tert-butyl (3S,10S,14S)-1-[(1r,4S)-4-(aminomethyl)cyclohexyl]-3-[(2-methylphenyl)methyl]-1,4,12-trioxo-2,5,11,13-tetraazahexadecane-10,14,16-tricarboxylate

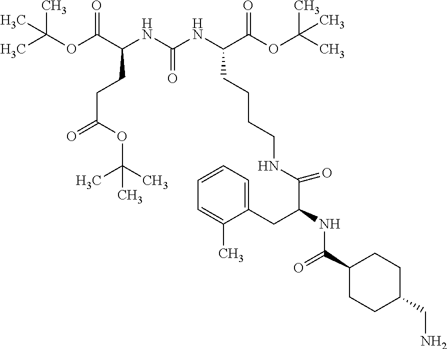 [SMILES: Cc1ccccc1C[C@H](NC(=O)[C@H]1CC[C@H](CN)CC1)C(=O)NCCCC[C@H](NC(=O)N[C@@H](CCC(=O)OC(C)(C)C)C(=O)OC(C)(C)C)C(=O)OC(C)(C)C]

tri-tert-butyl (3S,10S,14S)-1-{(1r,4S)-4-[({[(9H-fluoren-9-yl)methoxy]carbonyl}amino)methyl]cyclohexyl}-3-[(2-methylphenyl)methyl]-1,4,12-trioxo-2,5,11,13-tetraazahexadecane-10,14,16-tricarboxylate (53.0 mg, 80% purity, 42.0 μmol) was solubilised in DMF (1.1 ml), piperidine (83 μl, 840 μmol) was added and the mixture was stirred under argon at rt. The mixture was evaporated and purified by preparative HPLC (C18, acetonitrile/water with 0.1% formic acid) to give 50.0 mg (80% purity, 121% yield) of the target compound.

LC-MS (Method 1): Rₜ=1.02 min; MS (ESIpos): m/z=789 [M+H]⁺

### Example 12-A

### N⁶—{N-[(1r,4S)-4-(aminomethyl)cyclohexane-1-carbonyl]-2-methyl-L-phenylalanyl}-N²-{[(1S)-1,3-dicarboxypropyl]carbamoyl}-L-lysine

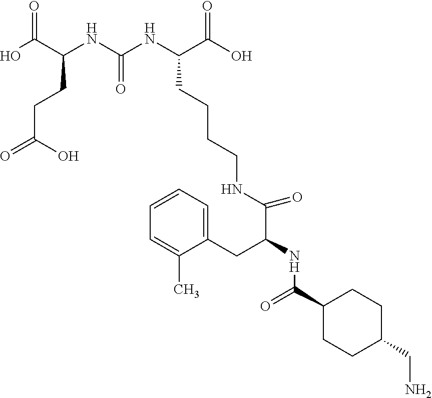 [SMILES: Cc1ccccc1C[C@H](NC(=O)[C@H]1CC[C@H](CN)CC1)C(=O)NCCCC[C@H](NC(=O)N[C@@H](CCC(=O)O)C(=O)O)C(=O)O]

tri-tert-butyl (3S,10S,14S)-1-[(1 r,4S)-4-(aminomethyl)cyclohexyl]-3-[(2-methyl]phenyl)methyl]-1,4,12-trioxo-2,5,11,13-tetraazahexadecane-10,14,16-tricarboxylate (12.0 mg, 98% purity, 14.9 μmol) was solubilised in DCM (920 μl), TFA (920 μl, 12 mmol) was added and the mixture was stirred under argon at rt. The mixture was evaporated and purified by preparative HPLC (C18, acetonitrile/water with 0.1% formic acid) to give 6.20 mg (80% purity, 54% yield) of the target compound.

LC-MS (Method 1): Rₜ=0.66 min; MS (ESIpos): m/z=621 [M+H]⁺

¹H-NMR (400 MHz, DMSO-d6) δ[ppm]: 0.884 (0.64), 0.916 (0.74), 1.156 (0.51), 1.210 (1.18), 1.233 (1.49), 1.300 (0.91), 1.314 (1.01), 1.437 (0.74), 1.566 (1.01), 1.606 (0.68), 1.675 (0.88), 1.702 (0.81), 1.751 (0.51), 1.790 (0.57), 1.807 (0.44), 1.829 (0.44), 2.074 (0.61), 2.096 (0.57), 2.130 (0.54), 2.155 (0.57), 2.172 (0.54), 2.248 (0.61), 2.269 (0.57), 2.288 (9.35), 2.332 (1.45), 2.518 (8.71), 2.522 (5.33), 2.539 (16.00), 2.601 (0.98), 2.673 (1.42), 2.678 (0.68), 2.728 (0.44), 2.751 (0.54), 2.762 (0.68), 2.786 (0.61), 2.921 (1.08), 2.936 (1.11), 2.956 (0.68), 2.971 (0.54), 3.053 (0.51), 3.068 (0.61), 3.086 (0.57), 3.101 (0.44), 3.330 (2.53), 3.918 (0.84), 3.930 (1.08), 4.421 (0.68), 4.435 (0.68), 6.117 (0.41), 6.392 (0.68), 6.411 (0.68), 7.039 (0.91), 7.050 (1.72), 7.057 (1.28), 7.061 (1.32), 7.066 (1.55), 7.079 (0.88), 7.087 (1.69), 7.103 (1.49), 7.122 (0.68), 7.825 (0.51), 7.839 (0.88), 8.083 (0.84), 8.104 (0.78).

#13 Linker

Intermediate 57

### tri-tert-butyl (5S,12S,16S)-1-(9H-fluoren-9-yl)-5-[(4-methylphenyl)methyl]-3,6,14-trioxo-2-oxa-4,7,13,15-tetraazaoctadecane-12,16,18-tricarboxylate

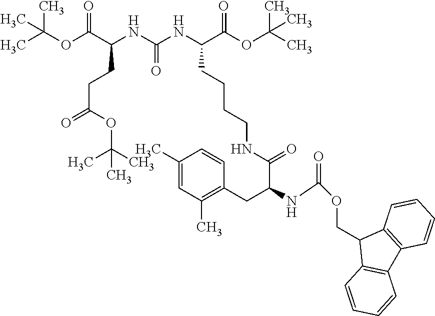 [SMILES: Cc1ccc(C[C@H](NC(=O)OCC2c3ccccc3-c3ccccc32)C(=O)NCCCC[C@H](NC(=O)N[C@@H](CCC(=O)OC(C)(C)C)C(=O)OC(C)(C)C)C(=O)OC(C)(C)C)c(C)c1]

di-tert-butyl N-{[(2S)-6-amino-1-tert-butoxy-1-oxohexan-2-yl]carbamoyl}-L-glutamate (300 mg, 70% purity, 431 μmol) and N-{[(9H-fluoren-9-yl)methoxy]carbonyl}-4-methyl-L-phenylalanine (259 mg, 646 μmol) were solubilised in DMF (3.3 ml), 4-methylmorpholine (140 μl, 1.3 mmol, CAS-RN: 109-02-4) and HATU (246 mg, 646 μmol) were added and the mixture was stirred under argon at rt. The mixture was evaporated and purified by preparative HPLC (C18, acetonitrile/water with 0.1% formic acid) to give 128 mg (98% purity, 33% yield) of the target compound.

LC-MS (Method 1): Rₜ=1.66 min; MS (ESIpos): m/z=872 [M+H]⁺

Intermediate 58

### di-tert-butyl (2S)-2-({[(2S)-6-{[(2S)-2-amino-3-(4-methylphenyl)propanoyl]amino}-1-tert-butoxy-1-oxohexan-2-yl]carbamoyl}amino)pentanedioate

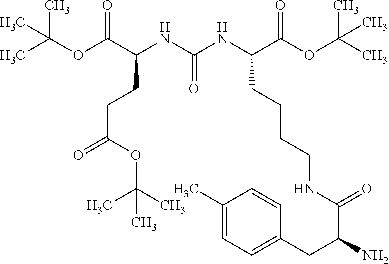 [SMILES: Cc1ccc(C[C@H](N)C(=O)NCCCC[C@H](NC(=O)N[C@@H](CCC(=O)OC(C)(C)C)C(=O)OC(C)(C)C)C(=O)OC(C)(C)C)cc1]

tri-tert-butyl (5S,12S,16S)-1-(9H-fluoren-9-yl)-5-[(4-methylphenyl)methyl]-3,6,14-trioxo-2-oxa-4,7,13,15-tetraazaoctadecane-12,16,18-tricarboxylate (128 mg, 98% purity, 144 μmol) was solubilised in DMF (1.5 ml), piperidine (280 μl, 2.9 mmol) was added and the mixture was stirred under argon overnight at rt. The mixture was evaporated and purified by preparative HPLC (C18, acetonitrile/water with 0.1% formic acid) to give 73.0 mg (98% purity, 77% yield) of the target compound.

LC-MS (Method 1): Rₜ=1.14 min; MS (ESIpos): m/z=650 [M+H]⁺

Intermediate 59

### tri-tert-butyl (3S,10S,14S)-1-{(1r,4S)-4-[({[(9H-fluoren-9-yl)methoxy]carbonyl}amino)methyl]cyclohexyl}-3-[(4-methylphenyl)methyl]-1,4,12-trioxo-2,5,11,13-tetraazahexadecane-10,14,16-tricarboxylate

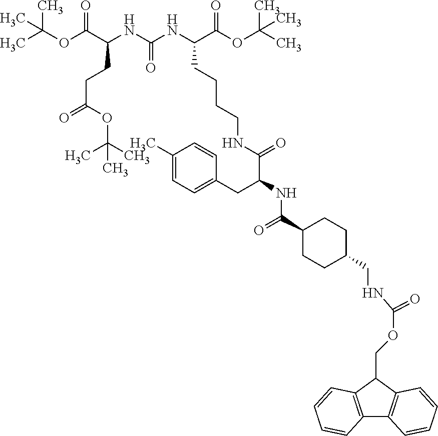 [SMILES: Cc1ccc(C[C@H](NC(=O)[C@H]2CC[C@H](CNC(=O)OCC3c4ccccc4-c4ccccc43)CC2)C(=O)NCCCC[C@H](NC(=O)N[C@@H](CCC(=O)OC(C)(C)C)C(=O)OC(C)(C)C)C(=O)OC(C)(C)C)cc1]

di-tert-butyl (2S)-2-({[(2S)-6-{[(2S)-2-amino-3-(4-methylphenyl)propanoyl]amino}-1-tert-butoxy-1-oxohexan-2-yl]carbamoyl}amino)pentanedioate (73.0 mg, 98% purity, 110 μmol) and (1r,4r)-4-[({[(9H-fluoren-9-yl)methoxy]carbonyl}amino)methyl]cyclohexane-1-carboxylic acid (62.8 mg, 165 μmol) were solubilised in DMF (850 μl), 4-methylmorpholine (36 μl, 330 μmol, CAS-RN: 109-02-4) and HATU (62.9 mg, 165 μmol) were added and the mixture was stirred under argon at rt. The mixture was evaporated and purified by preparative HPLC (C18, acetonitrile/water with 0.1% formic acid) to give 73.0 mg (98% purity, 64% yield) of the target compound.

LC-MS (Method 1): Rₜ=1.62 min; MS (ESIpos): m/z=1011 [M+H]⁺

Intermediate 60

### tri-tert-butyl (3S,10S,14S)-1-[(1r,4S)-4-(aminomethyl)cyclohexyl]-3-[(4-methylphenyl)methyl]-1,4,12-trioxo-2,5,11,13-tetraazahexadecane-10,14,16-tricarboxylate

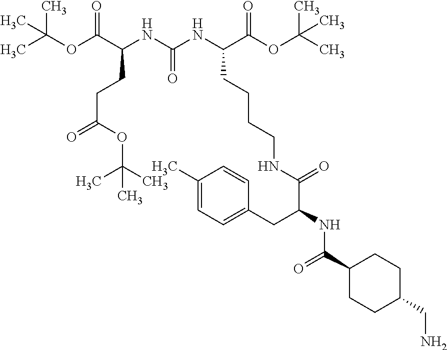 [SMILES: Cc1ccc(C[C@H](NC(=O)[C@H]2CC[C@H](CN)CC2)C(=O)NCCCC[C@H](NC(=O)N[C@@H](CCC(=O)OC(C)(C)C)C(=O)OC(C)(C)C)C(=O)OC(C)(C)C)cc1]

tri-tert-butyl (3S,10S,14S)-1-{(1r,4S)-4-[({[(9H-fluoren-9-yl)methoxy]carbonyl}amino)methyl]cyclohexyl}-3-[(4-methylphenyl)methyl]-1,4,12-trioxo-2,5,11,13-tetraazahexadecane-10,14,16-tricarboxylate (73.0 mg, 98% purity, 70.8 μmol) was solubilised in DMF (1.9 ml), piperidine (140 μl, 1.4 mmol) was added and the mixture was stirred under argon at rt. The mixture was evaporated and purified by preparative HPLC (C18, acetonitrile/water with 0.1% formic acid) to give 33.0 mg (80% purity, 47% yield) of the target compound.

LC-MS (Method 1): Rₜ=1.18 min; MS (ESIpos): m/z=789 [M+H]⁺

### Example 13-A

### N⁶—{N-[(1r,4S)-4-(aminomethyl)cyclohexane-1-carbonyl]-4-methyl-L-phenylalanyl}-N²-{[(1S)-1,3-dicarboxypropyl]carbamoyl}-L-lysine

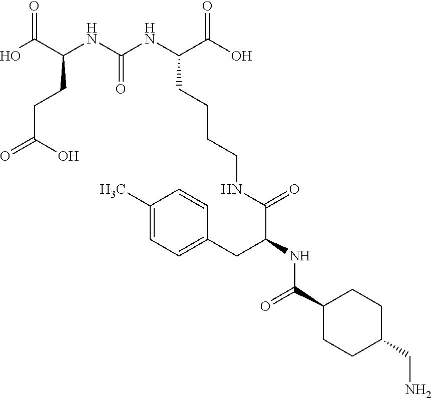 [SMILES: Cc1ccc(C[C@H](NC(=O)[C@H]2CC[C@H](CN)CC2)C(=O)NCCCC[C@H](NC(=O)N[C@@H](CCC(=O)O)C(=O)O)C(=O)O)cc1]

tri-tert-butyl (3S,10S,14S)-1-[(1r,4S)-4-(aminomethyl)cyclohexyl]-3-[(4-methylphenyl)methyl]-1,4,12-trioxo-2,5,11,13-tetraazahexadecane-10,14,16-tricarboxylate (10.8 mg, 98% purity, 13.4 μmol) was solubilised in DCM (820 μl), TFA (820 μl, 11 mmol) was added and the mixture was stirred under argon at rt. The mixture was evaporated and purified by preparative HPLC (C18, acetonitrile/water with 0.1% formic acid) to give 3.30 mg (90% purity, 36% yield) of the target compound.

LC-MS (Method 1): Rₜ=0.67 min; MS (ESIpos): m/z=621 [M+H]⁺

¹H-NMR (400 MHz, DMSO-d6) δ[ppm]: 0.882 (0.64), 0.913 (0.71), 1.153 (0.48), 1.210 (1.26), 1.232 (1.36), 1.296 (0.92), 1.313 (1.01), 1.439 (0.73), 1.543 (0.71), 1.565 (0.92), 1.603 (0.60), 1.624 (0.55), 1.672 (0.86), 1.701 (0.79), 1.751 (0.53), 1.783 (0.68), 1.799 (0.53), 1.821 (0.42), 2.074 (0.44), 2.098 (0.57), 2.128 (0.48), 2.159 (0.53), 2.173 (0.59), 2.241 (0.66), 2.263 (0.53), 2.288 (9.02), 2.331 (0.82), 2.518 (4.64), 2.522 (2.84), 2.539 (16.00), 2.601 (0.92), 2.673 (0.82), 2.727 (0.46), 2.749 (0.57), 2.761 (0.70), 2.784 (0.62), 2.896 (0.40), 2.921 (0.99), 2.934 (1.08), 2.955 (0.81), 2.969 (0.62), 3.042 (0.60), 3.058 (0.73), 3.074 (0.68), 3.091 (0.57), 3.349 (1.85), 3.915 (0.86), 3.929 (1.06), 4.422 (0.66), 4.438 (0.66), 6.135 (0.44), 6.388 (0.64), 6.407 (0.60), 7.038 (0.92), 7.042 (0.88), 7.049 (1.65), 7.056 (1.26), 7.061 (1.32), 7.065 (1.47), 7.079 (0.90), 7.086 (1.69), 7.102 (1.56), 7.122 (0.68), 7.854 (0.84), 8.094 (0.75), 8.115 (0.73).

#14 Linker

Intermediate 61

### tri-tert-butyl (3S,10S,14S)-1-[(1r,4S)-4-(aminomethyl)cyclohexyl]-3-[(1-bromonaphthalen-2-yl)methyl]-1,4,12-trioxo-2,5,11,13-tetraazahexadecane-10,14,16-tricarboxylate

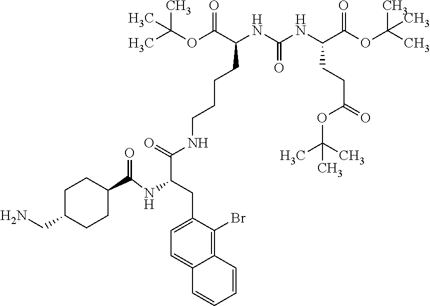 [SMILES: CC(C)(C)OC(=O)CC[C@H](NC(=O)N[C@@H](CCCCNC(=O)[C@H](Cc1ccc2ccccc2c1Br)NC(=O)[C@H]1CC[C@H](CN)CC1)C(=O)OC(C)(C)C)C(=O)OC(C)(C)C]

tri-tert-butyl (3S,10S,14S)-1-[(1r,4S)-4-(aminomethyl)cyclohexyl]-3-[(naphthalen-2-yl)methyl]-1,4,12-trioxo-2,5,11,13-tetraazahexadecane-10,14,16-tricarboxylate (32.0 mg, 38.8 μmol) was solubilised in acetonitrile (1.1 ml), N-bromosuccinimide (7.60 mg, 42.7 μmol) was added and the mixture was stirred for 3.5 h at rt. N-Bromosuccinimide (15.2 mg, 85.4 μmol) was added and the mixture was stirred in the dark over the weekend at rt. The mixture was filtered and evaporated. The residue was purified by preparative HPLC (C18, acetonitrile/water with 0.1% formic acid) to give 13.0 mg (37% yield) of the target compound.

LC-MS (Method 1): Rt=1.24 min; MS (ESIpos): m/z=903 [M+H]⁺

¹H-NMR (400 MHz, DMSO-d6) δ[ppm]: 1.232 (0.67), 1.353 (0.50), 1.382 (13.48), 1.389 (16.00), 1.907 (0.90), 2.327 (3.24), 2.331 (2.35), 2.336 (1.06), 2.518 (13.82), 2.523 (8.45), 2.562 (2.85), 2.669 (3.30), 2.673 (2.41), 2.678 (1.06), 7.868 (0.45).

### Example 14-A

### (3S,10S,14S)-1-[(1r,4S)-4-(aminomethyl)cyclohexyl]-3-[(1-bromonaphthalen-2-yl)methyl]-1,4,12-trioxo-2,5,11,13-tetraazahexadecane-10,14,16-tricarboxylic acid

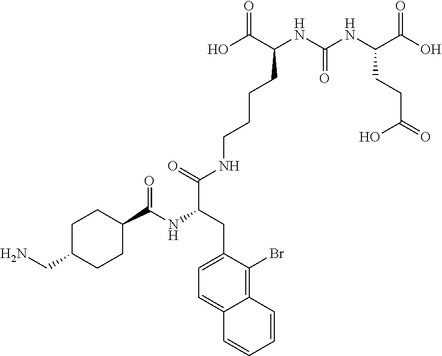 [SMILES: NC[C@H]1CC[C@H](C(=O)N[C@@H](Cc2ccc3ccccc3c2Br)C(=O)NCCCC[C@H](NC(=O)N[C@@H](CCC(=O)O)C(=O)O)C(=O)O)CC1]

tri-tert-butyl (3S,10S,14S)-1-[(1r,4S)-4-(aminomethyl)cyclohexyl]-3-[(1-bromonaphthalen-2-yl)methyl]-1,4,12-trioxo-2,5,11,13-tetraazahexadecane-10,14,16-tricarboxylate (14.0 mg, 98% purity, 15.2 μmol) was solubilised in DCM (930 μl), TFA (930 μl, 12 mmol) was added and the mixture was stirred under argon for 2 h at rt. The mixture was evaporated and purified by preparative HPLC (C18, acetonitrile/water with 0.1% formic acid) to give 10.0 mg (85% purity, 76% yield) of the target compound.

LC-MS (Method 1): Rₜ=1.00 min; MS (ESIpos): m/z=735 [M+H]⁺

¹H-NMR (400 MHz, DMSO-d6) δ[ppm]: 1.083 (3.33), 1.112 (3.88), 1.142 (4.07), 1.231 (14.61), 1.273 (6.57), 1.324 (7.86), 1.373 (6.20), 1.403 (6.84), 1.434 (6.10), 1.559 (7.68), 1.636 (9.90), 1.764 (6.01), 1.878 (4.07), 1.905 (4.81), 1.948 (3.98), 1.976 (3.70), 2.072 (2.87), 2.174 (6.01), 2.221 (7.95), 2.265 (5.92), 2.327 (8.51), 2.669 (6.75), 2.690 (3.14), 2.727 (11.84), 2.887 (13.32), 3.002 (16.00), 3.965 (4.53), 4.641 (3.14), 7.433 (4.53), 7.455 (4.81), 7.542 (3.24), 7.559 (5.46), 7.578 (4.07), 7.631 (3.98), 7.650 (5.27), 7.668 (3.14), 7.847 (6.20), 7.867 (5.92), 7.925 (7.21), 7.947 (7.58), 8.186 (7.58), 8.206 (7.40).

#15 Linker

Intermediate 62

### tri-tert-butyl (5S,12S,16S)-5-[(4-tert-butoxyphenyl)methyl]-3,6,14-trioxo-1-phenyl-2-oxa-4,7,13,15-tetraazaoctadecane-12,16,18-tricarboxylate

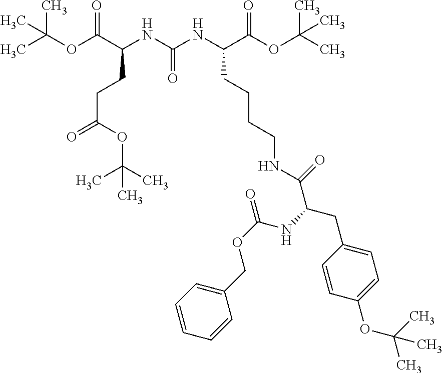 [SMILES: CC(C)(C)OC(=O)CC[C@H](NC(=O)N[C@@H](CCCCNC(=O)[C@H](Cc1ccc(OC(C)(C)C)cc1)NC(=O)OCc1ccccc1)C(=O)OC(C)(C)C)C(=O)OC(C)(C)C]

di-tert-butyl N-{[(2S)-6-amino-1-tert-butoxy-1-oxohexan-2-yl]carbamoyl}-L-glutamate (330 mg, 80% purity, 541 μmol) and N-[(benzyloxy)carbonyl]-O-tert-butyl-L-tyrosine (201 mg, 541 μmol; CAS-RN:[5545-54-0]) were solubilised in DMF (4.2 ml), 4-methylmorpholine (180 μl, 1.6 mmol, CAS-RN: 109-02-4) and HATU (309 mg, 812 μmol) were added and the mixture was stirred under argon for 3 h at rt. The mixture was evaporated and purified by preparative HPLC (C18, acetonitrile/water with 0.1% formic acid) to give 310 mg (100% purity, 68% yield) of the target compound.

LC-MS (Method 1): Rₜ=1.59 min; MS (ESpos): m/z=842 [M+H]⁺

Intermediate 63

### N⁶—{N-[(benzyloxy)carbonyl]-L-tyrosyl}-N²-{[(1S)-1,3-dicarboxypropyl]carbamoyl}-L-lysine

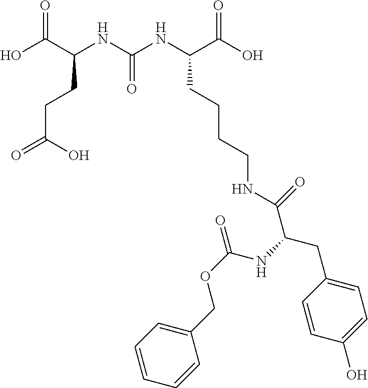 [SMILES: O=C(O)CC[C@H](NC(=O)N[C@@H](CCCCNC(=O)[C@H](Cc1ccc(O)cc1)NC(=O)OCc1ccccc1)C(=O)O)C(=O)O]

tri-tert-butyl (5S,12S,16S)-5-[(4-tert-butoxyphenyl)methyl]-3,6,14-trioxo-1-phenyl-2-oxa-4,7,13,15-tetraazaoctadecane-12,16,18-tricarboxylate (310 mg, 369 μmol) was solubilised in DCM (3.6 ml), TFA (1.4 ml, 18 mmol) was added and the mixture was stirred under argon overnight at rt. The mixture was evaporated and purified by preparative HPLC (C18, acetonitrile/water with 0.1% formic acid) to give 110 mg (90% purity, 44% yield) of the target compound.

LC-MS (Method 1): Rₜ=0.82 min; MS (ESIpos): m/z=617 [M+H]⁺

¹H-NMR (400 MHz, DMSO-d6) δ[ppm]: 1.239 (2.26), 1.257 (3.03), 1.276 (2.75), 1.295 (1.41), 1.344 (2.68), 1.362 (2.75), 1.397 (0.85), 1.478 (0.99), 1.497 (1.20), 1.511 (1.55), 1.530 (1.13), 1.619 (1.13), 1.634 (1.41), 1.654 (1.34), 1.681 (1.27), 1.695 (1.48), 1.715 (1.76), 1.730 (1.34), 1.751 (0.70), 1.866 (0.56), 1.883 (1.06), 1.900 (1.48), 1.917 (1.48), 1.934 (0.78), 1.953 (0.56), 2.074 (3.81), 2.169 (0.42), 2.183 (0.63), 2.210 (2.33), 2.226 (4.02), 2.231 (3.03), 2.248 (3.95), 2.266 (2.04), 2.290 (0.56), 2.318 (1.34), 2.518 (16.00), 2.523 (10.85), 2.539 (1.97), 2.584 (1.34), 2.609 (1.62), 2.618 (1.90), 2.644 (1.69), 2.678 (1.34), 2.786 (1.83), 2.797 (1.97), 2.820 (1.41), 2.832 (1.27), 2.957 (0.63), 2.972 (1.34), 2.990 (2.11), 3.004 (2.26), 3.022 (1.97), 3.038 (1.69), 3.055 (1.13), 3.072 (0.56), 4.010 (1.06), 4.031 (2.11), 4.044 (2.33), 4.065 (2.75), 4.079 (3.17), 4.086 (3.88), 4.099 (3.45), 4.106 (2.47), 4.119 (1.69), 4.900 (1.83), 4.933 (6.84), 4.949 (7.68), 4.981 (1.83), 6.286 (4.09), 6.306 (7.40), 6.327 (4.09), 6.623 (8.39), 6.644 (9.23), 7.020 (7.75), 7.042 (7.47), 7.144 (0.56), 7.226 (5.00), 7.244 (6.84), 7.247 (5.99), 7.266 (1.13), 7.283 (4.09), 7.297 (3.10), 7.301 (3.95), 7.317 (7.05), 7.331 (4.16), 7.335 (6.34), 7.352 (2.19), 7.357 (1.55), 7.366 (3.67), 7.388 (3.45), 7.927 (1.55), 7.941 (3.10), 7.955 (1.69), 9.169 (1.69), 12.519 (0.70).

Intermediate 64

### N⁶-L-tyrosyl-N²-{[(1S)-1,3-dicarboxypropyl]carbamoyl}-L-lysine

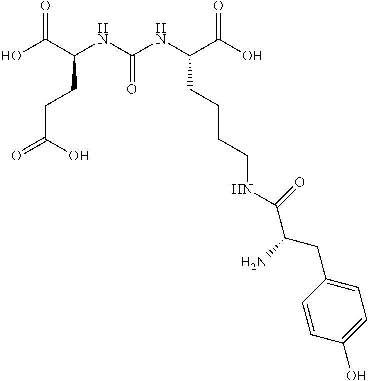 [SMILES: N[C@@H](Cc1ccc(O)cc1)C(=O)NCCCC[C@H](NC(=O)N[C@@H](CCC(=O)O)C(=O)O)C(=O)O]

N6-{N-[(benzyloxy)carbonyl]-L-tyrosyl}-N2-{[(1S)-1,3-dicarboxypropyl]carbamoyl}-L-lysine (110 mg, 178 μmol) was solubilised in MeOH (5 mL), Palladium on carbon (19.0 mg, 10% purity, 17.8 pmol) was added and the mixture was purged with hydrogen. The mixture was stirred for 3 h at rt under hydrogen atmosphere. The mixture was filtered over Celite, washed with MeOH and concentrated under reduced pressure to give 69.0 mg (80% yield) of the target compound.

Intermediate 65

### N⁶—(N-{(1r,4S)-4-[({[(9H-fluoren-9-yl)methoxy]carbonyl}amino)methyl]cyclohexane-1-carbonyl}-L-tyrosyl)-N²-{[(1S)-1,3-dicarboxypropyl]carbamoyl}-L-lysine

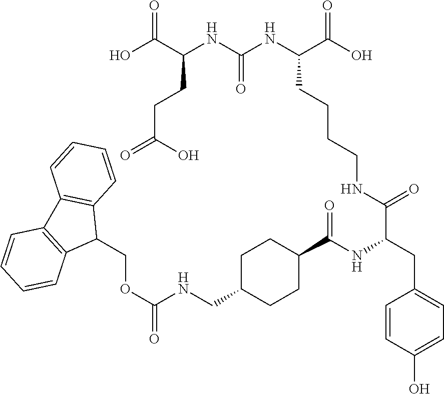 [SMILES: O=C(O)CC[C@H](NC(=O)N[C@@H](CCCCNC(=O)[C@H](Cc1ccc(O)cc1)NC(=O)[C@H]1CC[C@H](CNC(=O)OCC2c3ccccc3-c3ccccc32)CC1)C(=O)O)C(=O)O]

(1r,4r)-4-[({[(9H-fluoren-9-yl)methoxy]carbonyl}amino)methyl]cyclohexane-1-carboxylic acid (48.8 mg, 128 μmol) was solubilised in DMF (2.0 ml), 4-methylmorpholine (42 μl, 390 μmol, CAS-RN: 109-02-4) and HATU (48.9 mg, 128 μmol) were added and the mixture was stirred for 30 min at rt. N6-L-tyrosyl-N2-{[(1S)-1,3-dicarboxypropyl]carbamoyl}-L-lysine (62.0 mg, 128 μmol) was added and it was stirred under argon for 3 h at rt. The mixture was evaporated and purified by preparative HPLC (C18, acetonitrile/water with 0.1% formic acid) to give 72.0 mg (76% purity, 50% yield) of the target compound.

LC-MS (Method 1): Rt=1.09 min; MS (ESIpos): m/z=845 [M+H]⁺

### Example 15-A

### N⁶—{N-[(1r,4S)-4-(aminomethyl)cyclohexane-1-carbonyl]-L-tyrosyl}-N²-{[(1S)-1,3-dicarboxypropyl]carbamoyl}-L-lysine

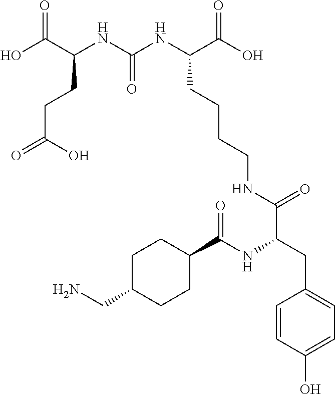 [SMILES: NC[C@H]1CC[C@H](C(=O)N[C@@H](Cc2ccc(O)cc2)C(=O)NCCCC[C@H](NC(=O)N[C@@H](CCC(=O)O)C(=O)O)C(=O)O)CC1]

N6-(N-{(1r,4S)-4-[({[(9H-fluoren-9-yl)methoxy]carbonyl}amino)methyl]cyclohexane-1-carbonyl}-L-tyrosyl)-N2-{[(1S)-1,3-dicarboxypropyl]carbamoyl}-L-lysine (70.0 mg, 82.9 μmol) was solubilised in DMF (640 μl), piperidine (160 μl, 1.7 mmol) was added and the mixture was stirred under argon overnight at rt. The mixture was evaporated and purified by preparative HPLC (C18, acetonitrile/water with 0.1% formic acid) to give 38.0 mg (99% purity, 73% yield) of the target compound.

¹H-NMR (400 MHz, DMSO-d6) δ[ppm]: 0.851 (0.57), 0.881 (0.92), 0.913 (0.92), 0.945 (0.42), 1.168 (0.71), 1.200 (1.27), 1.232 (2.34), 1.312 (1.13), 1.327 (1.20), 1.438 (0.92), 1.553 (1.06), 1.573 (1.35), 1.677 (0.99), 1.708 (0.92), 1.750 (0.57), 1.788 (0.78), 1.805 (0.57), 1.829 (0.50), 2.050 (0.42), 2.074 (1.77), 2.115 (0.50), 2.133 (0.42), 2.150 (0.64), 2.163 (0.71), 2.180 (0.42), 2.229 (0.50), 2.249 (0.71), 2.273 (0.57), 2.285 (0.42), 2.331 (3.12), 2.518 (16.00), 2.523 (10.41), 2.539 (7.65), 2.598 (1.70), 2.615 (2.12), 2.638 (0.78), 2.648 (0.99), 2.673 (3.75), 2.791 (0.78), 2.804 (0.85), 2.824 (0.64), 2.839 (0.57), 2.915 (0.57), 2.933 (0.64), 2.947 (0.50), 3.056 (0.57), 3.072 (0.71), 3.089 (0.64), 3.934 (1.27), 4.273 (0.42), 4.296 (0.78), 4.309 (0.78), 6.116 (0.50), 6.385 (0.92), 6.405 (0.85), 6.604 (3.75), 6.625 (3.89), 6.965 (3.54), 6.986 (3.12), 7.767 (0.64), 7.782 (1.06), 7.945 (1.13), 7.966 (1.06).

#16 Linker

Intermediate 66

### tri-tert-butyl (5S,12S,16S)-1-(9H-fluoren-9-yl)-3,6,14-trioxo-5-{[3-(pentafluoro-lambda⁶-sulfanyl)phenyl]methyl}-2-oxa-4,7,13,15-tetraazaoctadecane-12,16,18-tricarboxylate

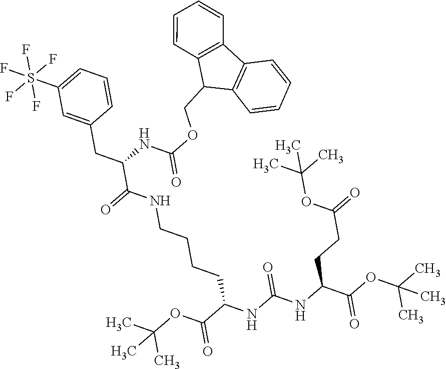 [SMILES: CC(C)(C)OC(=O)CC[C@H](NC(=O)N[C@@H](CCCCNC(=O)[C@H](Cc1cccc(S(F)(F)(F)(F)F)c1)NC(=O)OCC1c2ccccc2-c2ccccc21)C(=O)OC(C)(C)C)C(=O)OC(C)(C)C]

di-tert-butyl N-{[(2S)-6-amino-1-tert-butoxy-1-oxohexan-2-yl]carbamoyl}-L-glutamate (470 mg, 964 μmol) and N-{[(9H-fluoren-9-yl)methoxy]carbonyl}-3-(pentafluoro-lambda6-sulfanyl)phenylalanine (495 mg, 964 μmol) were solubilised in DMF (7.4 ml), 4-methylmorpholine (320 μl, 2.9 mmol, CAS-RN: 109-02-4) and HATU (550 mg, 1.45 mmol) were added and the mixture was stirred under argon overnight at rt. The mixture was evaporated and purified by preparative HPLC (C18, acetonitrile/water with 0.1% formic acid) to give 296 mg (87% purity, 27% yield) of the target compound.

LC-MS (Method 1): Rt=1.67 min; MS (ESIpos): m/z=984 [M+H]⁺

Intermediate 67

### di-tert-butyl (2S)-2-({[(2S)-6-({(2S)-2-amino-3-[3-(pentafluoro-lambda⁶-sulfanyl)phenyl]propanoyl}amino)-1-tert-butoxy-1-oxohexan-2-yl]carbamoyl}amino)pentanedioate

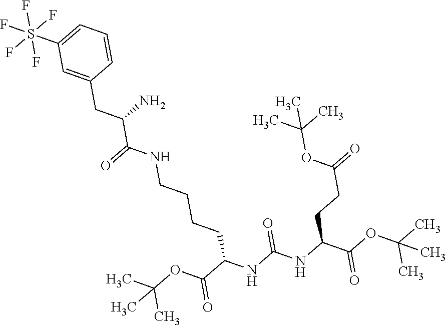 [SMILES: CC(C)(C)OC(=O)CC[C@H](NC(=O)N[C@@H](CCCCNC(=O)[C@@H](N)Cc1cccc(S(F)(F)(F)(F)F)c1)C(=O)OC(C)(C)C)C(=O)OC(C)(C)C]

tri-tert-butyl (5S,12S,16S)-1-(9H-fluoren-9-yl)-3,6,14-trioxo-5-{[3-(pentafluoro-lambda6-sulfanyl)phenyl]methyl}-2-oxa-4,7,13,15-tetraazaoctadecane-12,16,18-tricarboxylate (295 mg, 300 μmol) was solubilised in DMF (2.3 ml), piperidine (590 μl, 6.0 mmol) was added and the mixture was stirred under argon for 2 h at rt. The mixture was evaporated and purified by preparative HPLC (C18, acetonitrile/water with 0.1% formic acid) to give 210 mg (92% yield) of the target compound.

LC-MS (Method 1): Rₜ=1.25 min; MS (ESIpos): m/z=762 [M+H]⁺

Intermediate 68

### tri-tert-butyl (3S,10S,14S)-1-{(1r,4S)-4-[({[(9H-fluoren-9-yl)methoxy]carbonyl}amino)methyl]cyclohexyl}-1,4,12-trioxo-3-{[3-(pentafluoro-lambda⁶-sulfanyl)phenyl]methyl}-2,5,11,13-tetraazahexadecane-10,14,16-tricarboxylate

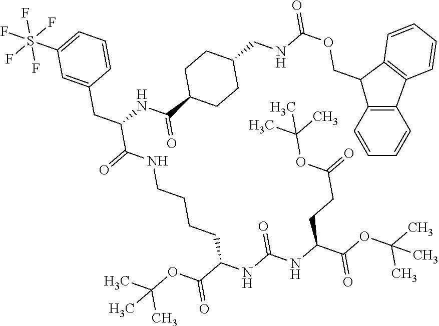 [SMILES: CC(C)(C)OC(=O)CC[C@H](NC(=O)N[C@@H](CCCCNC(=O)[C@H](Cc1cccc(S(F)(F)(F)(F)F)c1)NC(=O)[C@H]1CC[C@H](CNC(=O)OCC2c3ccccc3-c3ccccc32)CC1)C(=O)OC(C)(C)C)C(=O)OC(C)(C)C]

di-tert-butyl (2S)-2-({[(2S)-6-({(2S)-2-amino-3-[3-(pentafluoro-lambda6-sulfanyl)phenyl]propanoyl}amino)-1-tert-butoxy-1-oxohexan-2-yl]carbamoyl}amino)pentanedioate (240 mg, 315 μmol) and (1r,4r)-4-[({[(9H-fluoren-9-yl)methoxy]carbonyl}amino)methyl]cyclohexane-1-carboxylic acid (120 mg, 315 μmol) were solubilised in DMF (3.6 ml), 4-methylmorpholine (100 μl, 950 μmol, CAS-RN: 109-02-4) and HATU (126 mg, 331 μmol) were added and the mixture was stirred under argon overnight at rt. The mixture was evaporated and purified by preparative HPLC (C18, acetonitrile/water with 0.1% formic acid) to give 53.0 mg (99% purity, 15% yield) of the target compound.

LC-MS (Method 1): Rₜ=1.65 min; MS (ESIpos): m/z=1123 [M+H]⁺

¹H-NMR (400 MHz, DMSO-d6) δ[ppm]: 1.231 (0.49), 1.380 (16.00), 1.386 (14.23), 2.331 (0.96), 2.336 (0.43), 2.518 (5.06), 2.523 (3.34), 2.673 (0.96), 2.678 (0.43), 4.281 (0.67), 4.298 (0.45), 7.311 (0.74), 7.313 (0.69), 7.329 (0.47), 7.407 (0.63), 7.515 (0.65), 7.672 (0.61), 7.691 (0.58), 7.731 (0.52), 7.877 (0.67), 7.896 (0.63).

Intermediate 69

### tri-tert-butyl (3S,10S,14S)-1-[(1r,4S)-4-(aminomethyl)cyclohexyl]-1,4,12-trioxo-3-{[3-(pentafluoro-lambda⁶-sulfanyl)phenyl]methyl}-2,5,11,13-tetraazahexadecane-10,14,16-tricarboxylate

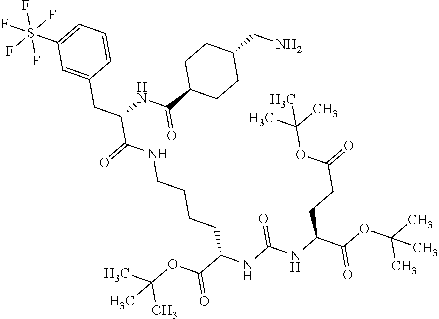 [SMILES: CC(C)(C)OC(=O)CC[C@H](NC(=O)N[C@@H](CCCCNC(=O)[C@H](Cc1cccc(S(F)(F)(F)(F)F)c1)NC(=O)[C@H]1CC[C@H](CN)CC1)C(=O)OC(C)(C)C)C(=O)OC(C)(C)C]

tri-tert-butyl (3S,10S,14S)-1-{(1r,4S)-4-[({[(9H-fluoren-9-yl)methoxy]carbonyl}amino)methyl]cyclohexyl}-1,4,12-trioxo-3-{[3-(pentafluoro-lambda6-sulfanyl)phenyl]methyl}-2,5,11,13-tetraazahexadecane-10,14,16-tricarboxylate (53.0 mg, 47.2 μmol) was solubilised in DMF (730 μl), piperidine (93 μl, 940 μmol) was added and the mixture was stirred under argon for 2 h at rt. The mixture was evaporated and purified by preparative HPLC (C18, acetonitrile/water with 0.1% formic acid) to give 23.0 mg (95% purity, 51% yield) of the target compound.

LC-MS (Method 1): Rt=1.25 min; MS (ESIpos): m/z=901 [M+H]⁺

¹H-NMR (400 MHz, DMSO-d6) δ[ppm]: 1.231 (0.43), 1.382 (13.02), 1.386 (16.00), 2.331 (0.77), 2.518 (4.42), 2.523 (2.75), 7.515 (0.68), 7.733 (0.43), 8.405 (0.63).

### Example 16-A

### N⁶—{N-[(1r,4S)-4-(aminomethyl)cyclohexane-1-carbonyl]-3-(pentafluoro-lambda⁶-sulfanyl)-L-phenylalanyl}-N²-{[(1S)-1,3-dicarboxypropyl]carbamoyl}-L-lysine

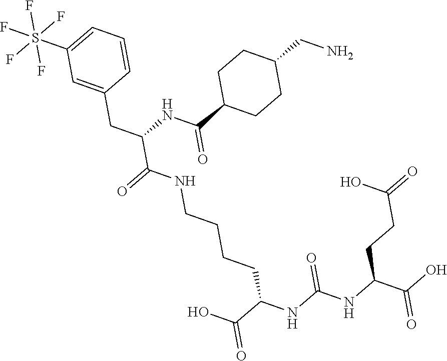 [SMILES: NC[C@H]1CC[C@H](C(=O)N[C@@H](Cc2cccc(S(F)(F)(F)(F)F)c2)C(=O)NCCCC[C@H](NC(=O)N[C@@H](CCC(=O)O)C(=O)O)C(=O)O)CC1]

tri-tert-butyl (3S,10S,14S)-1-[(1r,4S)-4-(aminomethyl)cyclohexyl]-1,4,12-trioxo-3-{[3-(pentafluoro-lambda6-sulfanyl)phenyl]methyl}-2,5,11,13-tetraazahexadecane-10,14,16-tricarboxylate (6.40 mg, 7.11 μmol) was solubilised in DCM (230 μl), TFA (270 μl, 3.6 mmol) was added and the mixture was stirred under argon for 3 h at rt. The mixture was evaporated and purified by preparative HPLC (C18, acetonitrile/water with 0.1% formic acid) to give 2.50 mg (99% purity, 48% yield) of the target compound.

LC-MS (Method 1): Rt=0.77 min; MS (ESIpos): m/z=733 [M+H]⁺

¹H-NMR (500 MHz, CHLOROFORM-d) 5 [ppm]: 7.263 (15.23), 7.278 (16.00), 7.288 (1.76), 7.292 (1.36), 7.499 (1.12), 7.658 (1.37).

#17 Linker

Intermediate 70

### tri-tert-butyl (5S,12S,16S)-1-(9H-fluoren-9-yl)-3,6,14-trioxo-5-[(quinolin-2-yl)methyl]-2-oxa-4,7,13,15-tetraazaoctadecane-12,16,18-tricarboxylate

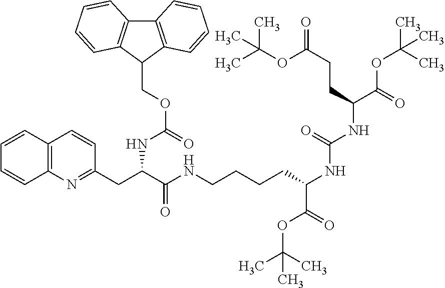 [SMILES: CC(C)(C)OC(=O)CC[C@H](NC(=O)N[C@@H](CCCCNC(=O)[C@H](Cc1ccc2ccccc2n1)NC(=O)OCC1c2ccccc2-c2ccccc21)C(=O)OC(C)(C)C)C(=O)OC(C)(C)C]

di-tert-butyl N-{[(2S)-6-amino-1-tert-butoxy-1-oxohexan-2-yl]carbamoyl}-L-glutamate (567 mg, 1.16 mmol) and N-{[(9H-fluoren-9-yl)methoxy]carbonyl}-3-quinolin-2-yl-L-alanine (510 mg, 1.16 mmol) were solubilised in DMF (8.9 ml), 4-methylmorpholine (320 μl, 2.9 mmol, CAS-RN: 109-02-4) and HATU (663 mg, 1.74 mmol) were added and the mixture was stirred under argon for 3 h at rt. The mixture was evaporated, diluted with water and extracted with DCM/isopropanol (4:1). The organic layer was dried, evaporated and purified by preparative HPLC (C18, acetonitrile/water with 0.1% formic acid) to give 433 mg (95% purity, 41% yield) of the target compound.

LC-MS (Method 1): Rt=1.59 min; MS (ESIpos): m/z=909 [M+H]⁺

¹H-NMR (400 MHz, DMSO-d6) δ[ppm]: 1.377 (16.00), 2.326 (0.51), 2.518 (2.42), 2.522 (1.48), 2.668 (0.51), 7.845 (0.41).

Intermediate 71

### di-tert-butyl (2S)-2-({[(2S)-6-{[(2S)-2-amino-3-(quinolin-2-yl)propanoyl]amino}-1-tert-butoxy-1-oxohexan-2-yl]carbamoyl}amino)pentanedioate

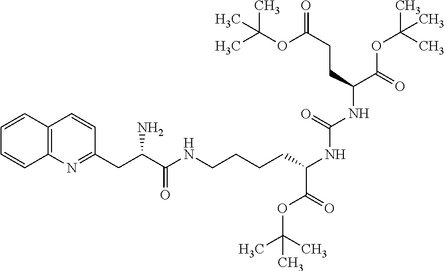 [SMILES: CC(C)(C)OC(=O)CC[C@H](NC(=O)N[C@@H](CCCCNC(=O)[C@@H](N)Cc1ccc2ccccc2n1)C(=O)OC(C)(C)C)C(=O)OC(C)(C)C]

tri-tert-butyl (5S,12S,16S)-1-(9H-fluoren-9-yl)-3,6,14-trioxo-5-[(quinolin-2-yl)methyl]-2-oxa-4,7,13,15-tetraazaoctadecane-12,16,18-tricarboxylate (366 mg, 403 μmol) was solubilised in DMF (3.1 ml), piperidine (800 μl, 8.1 mmol) was added and the mixture was stirred under argon for 2 h at rt. The mixture was evaporated and purified by preparative HPLC (C18, acetonitrile/water with 0.1% formic acid) to give 83.0 mg (100% purity, 51% yield) of the target compound.

LC-MS (Method 1): Rt=1.16 min; MS (ESIpos): m/z=687 [M+H]⁺

¹H-NMR (400 MHz, DMSO-d6) δ[ppm]: 1.381 (16.00), 1.385 (14.41), 1.387 (12.59), 2.518 (1.14), 2.522 (0.70), 7.412 (0.69), 7.433 (0.70), 7.543 (0.40), 7.936 (0.77), 7.940 (0.48), 7.954 (0.63), 8.245 (0.69), 8.266 (0.48).

Intermediate 72

### tri-tert-butyl (3S,10S,14S)-1-{(1r,4S)-4-[({[(9H-fluoren-9-yl)methoxy]carbonyl}amino)methyl]cyclohexyl}-1,4,12-trioxo-3-[(quinolin-2-yl)methyl]-2,5,11,13-tetraazahexadecane-10,14,16-tricarboxylate

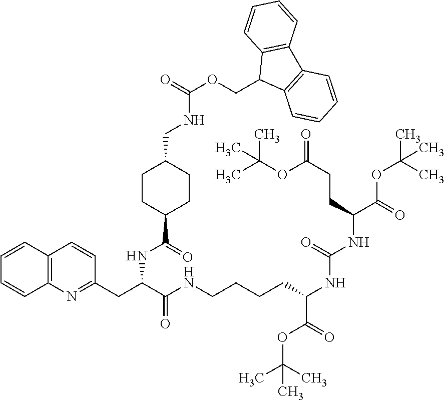 [SMILES: CC(C)(C)OC(=O)CC[C@H](NC(=O)N[C@@H](CCCCNC(=O)[C@H](Cc1ccc2ccccc2n1)NC(=O)[C@H]1CC[C@H](CNC(=O)OCC2c3ccccc3-c3ccccc32)CC1)C(=O)OC(C)(C)C)C(=O)OC(C)(C)C]

di-tert-butyl (2S)-2-({[(2S)-6-{[(2S)-2-amino-3-(quinolin-2-yl)propanoyl]amino}-1-tert-butoxy-1-oxohexan-2-yl]carbamoyl}amino)pentanedioate (67.0 mg, 97.7 μmol) and (1r,4r)-4-[({[(9H-fluoren-9-yl)methoxy]carbonyl}amino)methyl]cyclohexane-1-carboxylic acid (37.1 mg, 97.7 μmol) were solubilised in DMF (1.1 ml), 4-methylmorpholine (32 μl, 290 μmol, CAS-RN: 109-02-4) and HATU (39.0 mg, 103 μmol) were added and the mixture was stirred under argon overnight at rt. The mixture was evaporated and purified by preparative HPLC (C18, acetonitrile/water with 0.1% formic acid) to give 34.0 mg (95% purity, 32% yield) of the target compound.

¹H-NMR (400 MHz, DMSO-d6) δ[ppm]: 1.377 (13.17), 1.382 (16.00), 2.518 (1.44), 2.523 (0.95), 2.539 (1.28), 4.279 (0.52), 7.306 (0.50), 7.308 (0.48), 7.400 (0.49), 7.667 (0.47), 7.686 (0.44), 7.874 (0.51), 7.893 (0.49), 7.918 (0.45).

Intermediate 73

### tri-tert-butyl (3S,10S,14S)-1-[(1r,4S)-4-(aminomethyl)cyclohexyl]-1,4,12-trioxo-3-[(quinolin-2-yl)methyl]-2,5,11,13-tetraazahexadecane-10,14,16-tricarboxylate

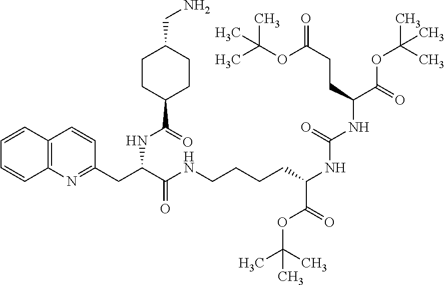 [SMILES: CC(C)(C)OC(=O)CC[C@H](NC(=O)N[C@@H](CCCCNC(=O)[C@H](Cc1ccc2ccccc2n1)NC(=O)[C@H]1CC[C@H](CN)CC1)C(=O)OC(C)(C)C)C(=O)OC(C)(C)C]

tri-tert-butyl (3S,10S,14S)-1-{(1r,4S)-4-[({[(9H-fluoren-9-yl)methoxy]carbonyl}amino)methyl]cyclohexyl}-1,4,12-trioxo-3-[(quinolin-2-yl)methyl]-2,5,11,13-tetraazahexadecane-10,14,16-tricarboxylate (63.0 mg, 60.2 μmol) was solubilised in DMF (1.9 ml), piperidine (120 μl, 1.2 mmol) was added and the mixture was stirred under argon for 1.5 h at rt. The mixture was evaporated and purified by preparative HPLC (C18, acetonitrile/water with 0.1% formic acid) to give 28.0 mg (100% purity, 56% yield) of the target compound.

LC-MS (Method 1): Rt=1.14 min; MS (ESIpos): m/z=826 [M+H]⁺

¹H-NMR (400 MHz, DMSO-d6) δ[ppm]: 1.380 (12.72), 1.384 (16.00), 2.518 (1.92), 2.522 (1.20), 3.290 (0.52), 7.407 (0.42), 7.429 (0.42), 8.424 (0.71).

Intermediate 74

### 2,2′-{[(3-{[2-({1-[2-({[(1S,4r)-4-{[(7S,11S,18S)-7,11-bis(tert-butoxycarbonyl)-2,2-dimethyl-4,9,17-trioxo-19-(quinolin-2-yl)-3-oxa-8,10,16-triazanonadecan-18-yl]carbamoyl}cyclohexyl]methyl}amino)-2-oxoethyl]-3-hydroxy-6-methyl-2-oxo-1,2-dihydropyridine-4-carbonyl}amino)ethyl](2-{[1-(carboxymethyl)-3-hydroxy-6-methyl-2-oxopyridine-4(2H)-carbonyl]amino}ethyl)amino}propyl)azanediyl]bis[(ethane-2,1-diyl)carbamoyl(3-hydroxy-6-methyl-2-oxopyridine-4,1(2H)-diyl)]}diacetic acid

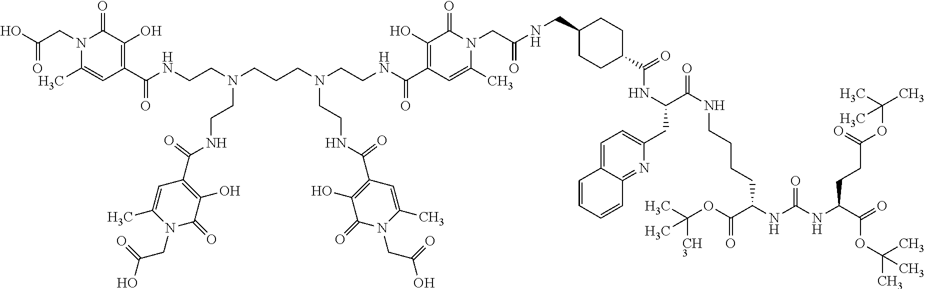 [SMILES: Cc1cc(C(=O)NCCN(CCCN(CCNC(=O)c2cc(C)n(CC(=O)O)c(=O)c2O)CCNC(=O)c2cc(C)n(CC(=O)NC[C@H]3CC[C@H](C(=O)N[C@@H](Cc4ccc5ccccc5n4)C(=O)NCCCC[C@H](NC(=O)N[C@@H](CCC(=O)OC(C)(C)C)C(=O)OC(C)(C)C)C(=O)OC(C)(C)C)CC3)c(=O)c2O)CCNC(=O)c2cc(C)n(CC(=O)O)c(=O)c2O)c(O)c(=O)n1CC(=O)O]

2,2′,2″,2′″-(propane-1,3-diylbis{nitrilobis[(ethane-2,1-diyl)carbamoyl(3-hydroxy-6-methyl-2-oxopyridine-4,1(2H)-diyl)]})tetraacetic acid (6.60 mg, 6.09 μmol) in 660 μL NMP tri-tert-butyl (3S,10S,14S)-1-[(1 r,4S)-4-(aminomethyl)cyclohexyl]-1,4,12-trioxo-3-[(quinolin-2-yl)methyl]-2,5,11,13-tetraazahexadecane-10,14,16-tricarboxylate (5.10 mg, 6.19 μmol) in 510 μL NMP and PyAOP (4.45 mg, 8.53 μmol) in 380 μL NMP are mixed in a vial. DIPEA (8.0 μL, 46.0 μmol) is added. Reaction mix is diluted with 38% ACN/water/0.1% TFA (8 mL) and the products purified by preparative HPLC (RP-HPLC using Äkta pure system, Column: Phenomenex Luna 5 μm C18(2) 100 Å, 250×21.2 mm Mobile phase: Water/0.1% TFA; ACN Gradient: 30-80% B over 40 min Flow: 10 mL/min Detection: UV 280/335 nm, tR product: 20 min) afforded 3.2 mg (28% yield) of the target product.

LC-MS (Method K, gradient: 10-70% B over 3 min): Rₜ=2.16 min; MS (ESIpos): m/z=1889.9 [M+H]⁺

### Example 17-C Monomer

### N⁶—{N-[(1r,4S)-4-({2-[4-({2-[{3-[bis(2-{[1-(carboxymethyl)-3-hydroxy-6-methyl-2-oxo-1,2-dihydropyridine-4-carbonyl]amino}ethyl)amino]propyl}(2-{[1-(carboxymethyl)-3-hydroxy-6-methyl-2-oxo-1,2-dihydropyridine-4-carbonyl]amino}ethyl)amino]ethyl}carbamoyl)-3-hydroxy-6-methyl-2-oxopyridin-1(2H)-yl]acetamido}methyl)cyclohexane-1-carbonyl]-3-(quinolin-2-yl)-L-alanyl}-N²-{[(1S)-1,3-dicarboxypropyl]carbamoyl}-L-lysine

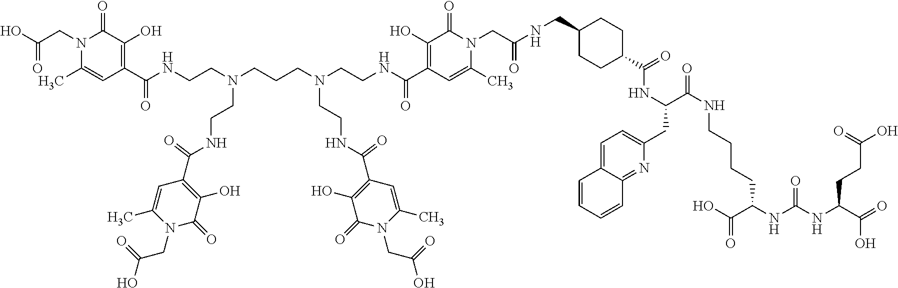 [SMILES: Cc1cc(C(=O)NCCN(CCCN(CCNC(=O)c2cc(C)n(CC(=O)O)c(=O)c2O)CCNC(=O)c2cc(C)n(CC(=O)NC[C@H]3CC[C@H](C(=O)N[C@@H](Cc4ccc5ccccc5n4)C(=O)NCCCC[C@H](NC(=O)N[C@@H](CCC(=O)O)C(=O)O)C(=O)O)CC3)c(=O)c2O)CCNC(=O)c2cc(C)n(CC(=O)O)c(=O)c2O)c(O)c(=O)n1CC(=O)O]

2,2′-{[(3-{[2-({1-[2-({[(1S,4r)-4-{[(7S,11S,18S)-7,11-bis(tert-butoxycarbonyl)-2,2-dimethyl-4,9,17-trioxo-19-(quinolin-2-yl)-3-oxa-8,10,16-triazanonadecan-18-yl]carbamoyl}cyclohexyl]methyl}amino)-2-oxoethyl]-3-hydroxy-6-methyl-2-oxo-1,2-dihydropyridine-4-carbonyl}amino)ethyl](2-{[1-(carboxymethyl)-3-hydroxy-6-methyl-2-oxopyridine-4(2H)-carbonyl]amino}ethyl)amino}propyl)azanediyl]bis[(ethane-2,1-diyl)carbamoyl(3-hydroxy-6-methyl-2-oxopyridine-4,1(2H)-diyl)]}diacetic acid (5.5 mg, 2.9 μmol) is treated with 80% TFA in water (0.5 mL). Water (18 mL) is added and the reaction mixture is lyophilised affording 5.2 mg (100% yield)

LC-MS (Method K, gradient: 10-50% B over 3 min): Rₜ=1.61 min; MS (ESIpos): m/z=1721.8 [M+H]⁺

### Example 17-C Monomer-²³²Th

### [N⁶—{N-[(1r,4S)-4-({2-[4-({2-[{3-[bis(2-{[1-(carboxymethyl)-3-(hydroxy-kappaO)-6-methyl-2-(oxo-kappaO)-1,2-dihydropyridine-4-carbonyl]amino}ethyl)amino]propyl}(2-{[1-(carboxymethyl)-3-(hydroxy-kappaO)-6-methyl-2-(oxo-kappaO)-1,2-dihydropyridine-4-carbonyl]amino}ethyl)amino]ethyl}carbamoyl)-3-(hydroxy-kappaO)-6-methyl-2-(oxo-kappaO)pyridin-1(2H)-yl]acetamido}methyl)cyclohexane-1-carbonyl]-3-(quinolin-2-yl)-L-alanyl}-N²-{[(1S)-1,3-dicarboxypropyl]carbamoyl}-L-lysinato(4−)](²³²Th)Thorium

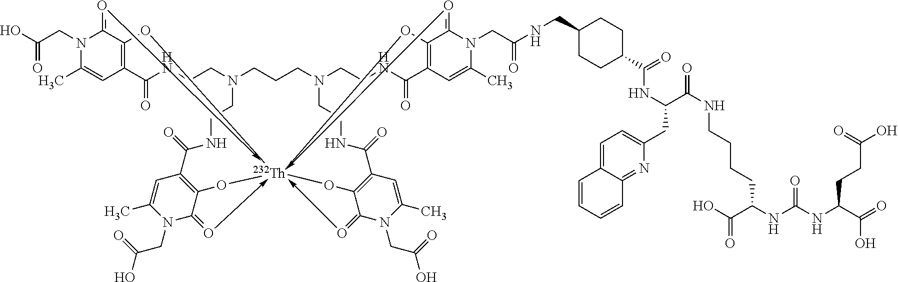

N⁶—{N-[(1r,4S)-4-({2-[4-({2-[{3-[bis(2-{[1-(carboxymethyl)-3-hydroxy-6-methyl-2-oxo-1,2-dihydropyridine-4-carbonyl]amino}ethyl)amino]propyl}(2-{[1-(carboxymethyl)-3-hydroxy-6-methyl-2-oxo-1,2-dihydropyridine-4-carbonyl]amino}ethyl)amino]ethyl}carbamoyl)-3-hydroxy-6-methyl-2-oxopyridin-1(2H)-yl]acetamido}methyl)cyclohexane-1-carbonyl]-3-(quinolin-2-yl)-L-alanyl}-N²-{[(1S)-1,3-dicarboxypropyl]carbamoyl}-L-lysine (1.00 mg, 0.581 μmol) is dissolved in 0.1 M TRIS buffer (420 μL). Th-232 solution (119 μL, 1 μg/μl in HNO₃ [2%])) is added. Reaction mixture is lyophilised affording 1.10 mg (95% purity, 92% yield)

LC-MS (Method K, gradient: 10-50% B over 3 min): Rₜ=1.38 min; MS (ESIpos): m/z=1949.8 [M+H]⁺

### Example 17-C Monomer-²²⁷Th

### [N⁶—{N-[(1r,4S)-4-({2-[4-({2-[{3-[bis(2-{[1-(carboxymethyl)-3-(hydroxy-kappaO)-6-methyl-2-(oxo-kappaO)-1,2-dihydropyridine-4-carbonyl]amino}ethyl)amino]propyl}(2-{[1-(carboxymethyl)-3-(hydroxy-kappaO)-6-methyl-2-(oxo-kappaO)-1,2-dihydropyridine-4-carbonyl]amino}ethyl)amino]ethyl}carbamoyl)-3-(hydroxy-kappaO)-6-methyl-2-(oxo-kappaO)pyridin-1(2H)-yl]acetamido}methyl)cyclohexane-1-carbonyl]-3-(quinolin-2-yl)-L-alanyl}-N²-{[(1S)-1,3-dicarboxypropyl]carbamoyl}-L-lysinato(4−)](²²⁷Th)Thorium

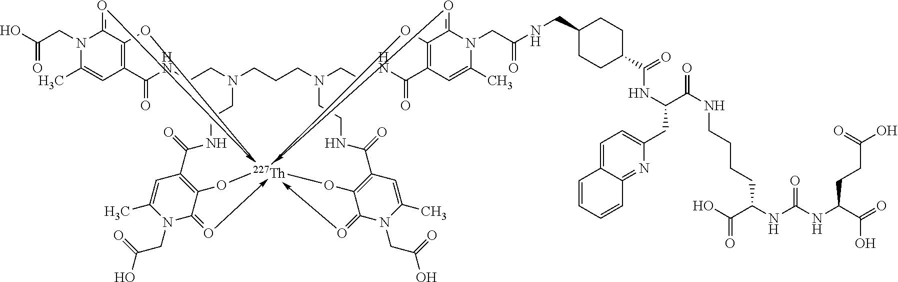

N⁶—{N-[(1r,4S)-4-({2-[4-({2-[{3-[bis(2-{[1-(carboxymethyl)-3-hydroxy-6-methyl-2-oxo-1,2-dihydropyridine-4-carbonyl]amino}ethyl)amino]propyl}(2-{[1-(carboxymethyl)-3-hydroxy-6-methyl-2-oxo-1,2-dihydropyridine-4-carbonyl]amino}ethyl)amino]ethyl}carbamoyl)-3-hydroxy-6-methyl-2-oxopyridin-1(2H)-yl]acetamido}methyl)cyclohexane-1-carbonyl]-3-(quinolin-2-yl)-L-alanyl}-N²-{[(1S)-1,3-dicarboxypropyl]carbamoyl}-L-lysine (5.3 μg) dissolved in 184 μL 30 mM citrate buffer (pH 5.5) containing 0.5 mg/mL pABA was mixed with thorium-227 in 0.5 M HCl (2 μL) at 0.3 MBq/nmol specific activity and RAC of 4.5 MBq/mL. 1M carbonate buffer pH 9 (18 μL) was added and mixture incubated for 2 hrs. The labelling efficiency was determined to be 94% by iTLC.

### Example 17-A

### N⁶—{N-[(1r,4S)-4-(aminomethyl)cyclohexane-1-carbonyl]-3-(quinolin-2-yl)-L-alanyl}-N²-{[(1S)-1,3-dicarboxypropyl]carbamoyl}-L-lysine

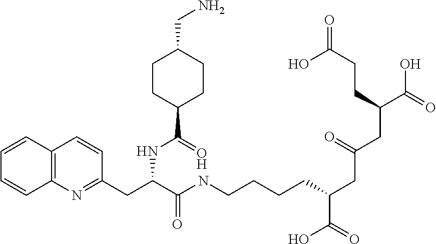 [SMILES: NC[C@H]1CC[C@H](C(=O)N[C@@H](Cc2ccc3ccccc3n2)C(=O)NCCCC[C@H](CC(=O)C[C@@H](CCC(=O)O)C(=O)O)C(=O)O)CC1]

tri-tert-butyl (3S,10S,14S)-1-[(1r,4S)-4-(aminomethyl)cyclohexyl]-1,4,12-trioxo-3-[(quinolin-2-yl)methyl]-2,5,11,13-tetraazahexadecane-10,14,16-tricarboxylate (6.00 mg, 7.27 μmol) was solubilised in DCM (230 μl), TFA (280 μl, 3.6 mmol) was added and the mixture was stirred under argon for 3 h at rt. The mixture was evaporated and purified by preparative HPLC (C18, acetonitrile/water with 0.1% formic acid) to give 2.80 mg (99% purity, 58% yield) of the target compound.

LC-MS (Method 1): Rt=0.55 min; MS (ESIpos): m/z=658 [M+H]⁺

¹H-NMR (500 MHz, DMSO-d6) δ[ppm]: 0.875 (4.33), 1.030 (1.31), 1.055 (3.10), 1.079 (3.02), 1.101 (1.31), 1.170 (1.47), 1.196 (4.16), 1.230 (9.31), 1.242 (7.76), 1.286 (1.80), 1.300 (3.76), 1.314 (6.94), 1.329 (8.00), 1.342 (5.22), 1.415 (4.49), 1.425 (4.90), 1.439 (4.73), 1.454 (3.35), 1.490 (3.92), 1.515 (3.43), 1.556 (2.78), 1.566 (3.59), 1.582 (3.02), 1.634 (3.43), 1.667 (5.80), 1.696 (5.88), 1.713 (5.14), 1.725 (5.80), 1.751 (6.04), 1.762 (4.65), 1.777 (4.00), 1.790 (2.94), 1.804 (2.04), 2.042 (2.37), 2.066 (4.08), 2.089 (2.12), 2.184 (3.76), 2.200 (4.73), 2.209 (4.73), 2.225 (5.14), 2.240 (3.18), 2.254 (2.04), 2.271 (1.14), 2.358 (3.59), 2.361 (4.90), 2.365 (3.67), 2.515 (16.00), 2.518 (13.80), 2.522 (10.69), 2.540 (2.04), 2.598 (7.10), 2.632 (4.33), 2.635 (5.55), 2.639 (4.16), 2.693 (0.73), 2.727 (3.92), 2.888 (4.98), 2.952 (3.02), 2.964 (4.16), 2.978 (4.90), 2.989 (4.33), 3.044 (4.57), 3.057 (5.39), 3.070 (4.73), 3.083 (3.59), 3.108 (5.06), 3.127 (5.80), 3.135 (6.53), 3.154 (6.12), 3.285 (7.10), 3.295 (7.92), 3.312 (6.94), 3.323 (6.37), 3.505 (3.43), 3.944 (6.04), 3.954 (7.10), 3.963 (7.10), 4.719 (2.86), 4.737 (5.14), 4.747 (5.14), 4.765 (2.61), 6.188 (3.43), 6.381 (4.16), 6.395 (3.92), 7.421 (11.27), 7.438 (11.10), 7.523 (5.22), 7.539 (9.71), 7.553 (6.04), 7.699 (5.14), 7.701 (5.14), 7.715 (8.73), 7.731 (4.98), 7.875 (3.84), 7.887 (6.69), 7.898 (4.33), 7.907 (11.10), 7.917 (13.31), 7.934 (10.04), 7.950 (0.65), 8.149 (5.88), 8.166 (5.55), 8.231 (15.51), 8.238 (11.18), 8.247 (13.39).

Intermediate 75

### ethyl (1r,4r)-4-({2-[4,7,10-tris(2-tert-butoxy-2-oxoethyl)-1,4,7,10-tetraazacyclododecan-1-yl]acetamido}methyl)cyclohexane-1-carboxylate

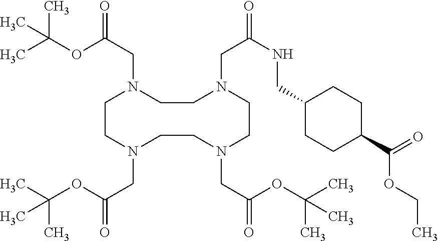 [SMILES: CCOC(=O)[C@H]1CC[C@H](CNC(=O)CN2CCN(CC(=O)OC(C)(C)C)CCN(CC(=O)OC(C)(C)C)CCN(CC(=O)OC(C)(C)C)CC2)CC1]

[4,7,10-tris(2-tert-butoxy-2-oxoethyl)-1,4,7,10-tetraazacyclododecan-1-yl]acetic acid (1.00 g, 1.75 mmol), [(1H-benzotriazol-1-yl)oxy](dimethylamino)-N,N-dimethylmethaniminium hexafluoridophosphate(1−) (662 mg, 1.75 mmol; CAS-RN:94790-37-1) and N,N-diisopropylethylamine (820 μl, 4.8 mmol) were stirred in DMF (12 ml) for 30 min at rt. ethyl (1r,4r)-4-(aminomethyl)cyclohexane-1-carboxylate (294 mg, 1.59 mmol) was added and the mixture was stirred overnight at rt. The mixture was diluted with water and extracted with DCM. The organic phase was washed with brine, dried and evaporated to give 160.0 mg (14% yield) of the target compound.

LC-MS (Method 3): Rt=1.36 min; MS (ESIpos): m/z=741 [M+H]⁺

Intermediate 76

### (1r,4r)-4-({2-[4,7,10-tris(2-tert-butoxy-2-oxoethyl)-1,4,7,10-tetraazacyclododecan-1-yl]acetamido}methyl)cyclohexane-1-carboxylic acid

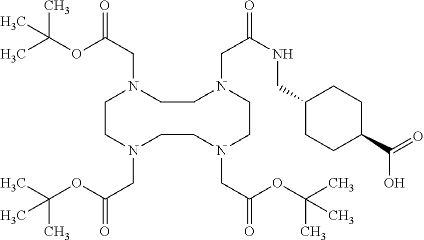 [SMILES: CC(C)(C)OC(=O)CN1CCN(CC(=O)NC[C@H]2CC[C@H](C(=O)O)CC2)CCN(CC(=O)OC(C)(C)C)CCN(CC(=O)OC(C)(C)C)CC1]

ethyl (1r,4r)-4-({2-[4,7,10-tris(2-tert-butoxy-2-oxoethyl)-1,4,7,10-tetraazacyclododecan-1-yl]acetamido}methyl)cyclohexane-1-carboxylate (270 mg, 365 μmol) was solubilised in MeOH (2.2 ml), lithium hydroxide (3.6 ml, 1.0 M, 3.6 mmol) was added and the mixture was stirred overnight at rt. The mixture was neutralized with HCl (1.0 M) and extracted with DCM/MeOH (4:1). The organic layer was dried and evaporated to give 150 mg (85% purity, 80% yield) of the target compound.

LC-MS (Method 3): Rt=0.93 min; MS (ESIpos): m/z=713 [M+H]⁺

Intermediate 77

### tri-tert-butyl (3S,10S,14S)-1,4,12-trioxo-3-[(quinolin-2-yl)methyl]-1-[(1r,4S)-4-({2-[4,7,10-tris(2-tert-butoxy-2-oxoethyl)-1,4,7,10-tetraazacyclododecan-1-yl]acetamido}methyl)cyclohexyl]-2,5,11,13-tetraazahexadecane-10,14,16-tricarboxylate

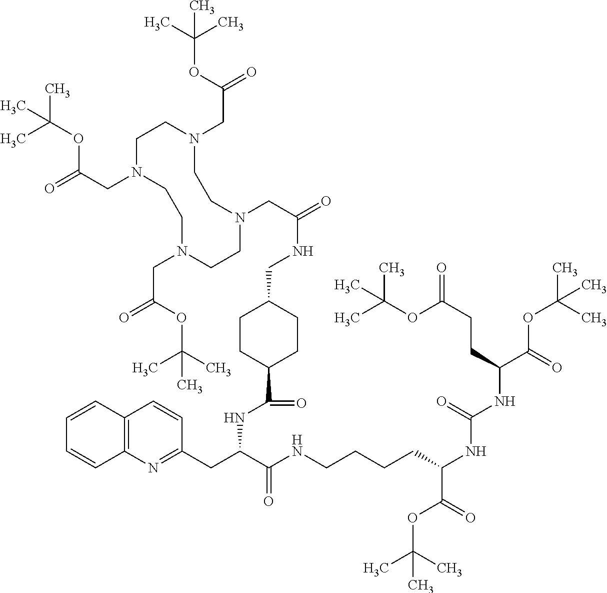 [SMILES: CC(C)(C)OC(=O)CC[C@H](NC(=O)N[C@@H](CCCCNC(=O)[C@H](Cc1ccc2ccccc2n1)NC(=O)[C@H]1CC[C@H](CNC(=O)CN2CCN(CC(=O)OC(C)(C)C)CCN(CC(=O)OC(C)(C)C)CCN(CC(=O)OC(C)(C)C)CC2)CC1)C(=O)OC(C)(C)C)C(=O)OC(C)(C)C]

(1 r,4r)-4-({2-[4,7,10-tris(2-tert-butoxy-2-oxoethyl)-1,4,7,10-tetraazacyclododecan-1-yl]acetamido}methyl)cyclohexane-1-carboxylic acid (71.6 mg, 101 μmol) was solubilised in DMF (770 μl), 4-methylmorpholine (33 μl, 300 μmol, CAS-RN: 109-02-4) and HATU (45.9 mg, 121 μmol) were added and the mixture was stirred for 20 min at rt. di-tert-butyl (2S)-2-({[(2S)-6-{[(2S)-2-amino-3-(quinolin-2-yl)propanoyl]amino}-1-tert-butoxy-1-oxohexan-2-yl]carbamoyl}amino)pentanedioate (69.0 mg, 101 μmol) was added and it was stirred under argon for 3 h at rt. The mixture was evaporated, diluted with water and extracted with DCM/isopropanol (4:1). The organic phase was dried, evaporated and purified by preparative HPLC (C18, acetonitrile/water with 0.1% formic acid) to give 138 mg (99% yield) of the target compound.

### Example 17-B

### N⁶-{3-(quinolin-2-yl)-N-[(1r,4S)-4-({2-[4,7,10-tris(carboxymethyl)-1,4,7,10-tetraazacyclododecan-1-yl]acetamido}methyl)cyclohexane-1-carbonyl]-L-alanyl}-N²-{[(1S)-1,3-dicarboxypropyl]carbamoyl}-L-lysine

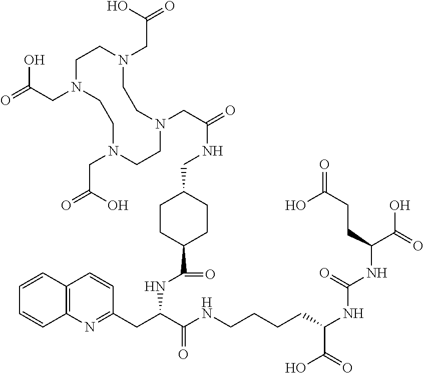 [SMILES: O=C(O)CC[C@H](NC(=O)N[C@@H](CCCCNC(=O)[C@H](Cc1ccc2ccccc2n1)NC(=O)[C@H]1CC[C@H](CNC(=O)CN2CCN(CC(=O)O)CCN(CC(=O)O)CCN(CC(=O)O)CC2)CC1)C(=O)O)C(=O)O]

tri-tert-butyl (3S,10S,14S)-1,4,12-trioxo-3-[(quinolin-2-yl)methyl]-1-[(1r,4S)-4-({2-[4,7,10-tris(2-tert-butoxy-2-oxoethyl)-1,4,7,10-tetraazacyclododecan-1-yl]acetamido}methyl)cyclohexyl]-2,5,11,13-tetraazahexadecane-10,14,16-tricarboxylate (100 mg, 72.5 μmol) was solubilised in DCM (2.8 ml), TFA (2.8 ml, 36 mmol) was added and the mixture was stirred under argon for 4.5 h at rt. The mixture was evaporated and purified by preparative HPLC (C18, acetonitrile/water with 0.1% formic acid) to give 20.0 mg (95% purity, 25% yield) of the target compound.

LC-MS (Method 1): Rt=0.56 min; MS (ESIneg): m/z=1042 [M−H]⁻

¹H-NMR (400 MHz, DMSO-d6) δ[ppm]: 0.834 (0.51), 0.852 (0.45), 1.232 (1.73), 1.323 (1.16), 1.368 (0.51), 1.385 (1.29), 1.400 (0.77), 1.421 (0.39), 1.478 (0.71), 1.579 (0.71), 1.681 (0.90), 1.695 (0.84), 1.716 (0.64), 1.738 (1.09), 1.905 (0.51), 1.922 (0.58), 2.047 (0.45), 2.210 (0.77), 2.226 (1.16), 2.246 (1.22), 2.268 (0.71), 2.337 (1.35), 2.518 (15.87), 2.523 (10.41), 2.540 (2.44), 2.679 (1.41), 2.687 (0.96), 2.728 (1.73), 2.787 (0.71), 2.889 (2.57), 2.962 (3.28), 3.091 (0.58), 3.114 (0.77), 3.126 (0.77), 3.147 (0.84), 3.498 (2.06), 3.577 (0.51), 3.653 (0.45), 3.994 (0.64), 4.006 (0.64), 4.092 (0.71), 4.105 (0.64), 4.721 (0.45), 4.734 (0.51), 6.275 (0.90), 6.295 (0.84), 6.310 (1.03), 6.330 (0.96), 7.411 (1.61), 7.432 (1.54), 7.520 (0.58), 7.540 (1.16), 7.558 (0.77), 7.697 (0.58), 7.701 (0.64), 7.718 (1.09), 7.739 (0.71), 7.908 (1.80), 7.916 (1.67), 7.924 (1.29), 7.938 (1.54), 8.079 (0.64), 8.134 (16.00), 8.231 (1.35), 8.252 (1.67), 8.273 (0.39).

### Example 17-B-²³²Th

### Thorium-{N⁶-[3-(quinolin-2-yl)-N-{4-[(2-{4,7,10-tris[(carboxy-kappaO)methyl]-1,4,7,10-tetraazacyclododecan-1-yl-kappa⁴N¹,N⁴,N⁷,N¹⁰}acetamido)methyl]cyclohexane-1-carbonyl}-L-alanyl]-N²-[(1,3-dicarboxypropyl)carbamoyl]-L-lysinate

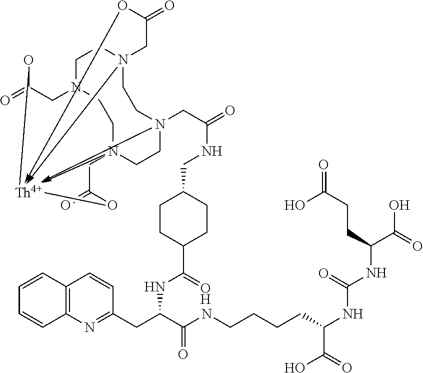

N⁶-{3-(quinolin-2-yl)-N-[(1r,4S)-4-({2-[4,7,10-tris(carboxymethyl)-1,4,7,10-tetraazacyclododecan-1-yl]acetamido}methyl)cyclohexane-1-carbonyl]-L-alanyl}-N²-{[(1S)-1,3-dicarboxypropyl]carbamoyl}-L-lysine (5.00 mg, 4.79 μmol) was solubilised in ammonium acetate (5.0 ml, 5.0 mmol, 1.0 M, prepared with ultrafiltered and autoclaved water), thorium solution (1.1 ml, 4.8 μmol, 1 μg/μl in HNO₃ [2%]) was added and the mixture was stirred for 3.5 hrs at 90° C. The mixture was diluted with water and lyophilized. The residue was purified by preparative HPLC (C18, acetonitrile/water with 0.1% formic acid) to give 2.30 mg (95% purity, 36% yield) of the target compound.

LC-MS (Method 1): Rt=0.54 min; MS (ESIpos): m/z=1272 [M+H]⁺

### Example 17-B-²²⁷Th

### [²²⁷Th]Thorium-{N⁶-[3-(quinolin-2-yl)-N-{(1r,4S)-4-[(2-{4,7,10-tris[(carboxy-kappaO)methyl]-1,4,7,10-tetraazacyclododecan-1-yl-kappa⁴N¹,N⁴,N⁷,N¹⁰}acetamido)methyl]cyclohexane-1-carbonyl}-L-alanyl]-N²-{[(1S)-1,3-dicarboxypropyl]carbamoyl}-L-lysinate}

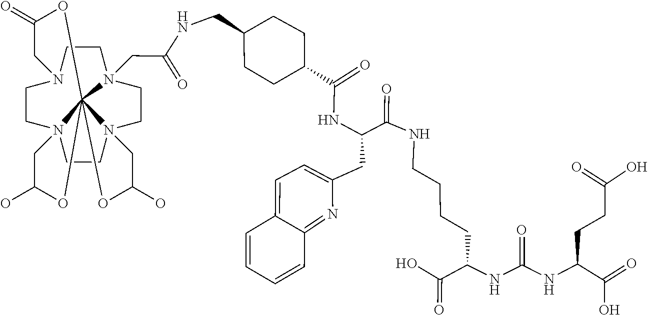

N⁶-{3-(quinolin-2-yl)-N-[(1r,4S)-4-({2-[4,7,10-tris(carboxymethyl)-1,4,7,10-tetraazacyclododecan-1-yl]acetamido}methyl)cyclohexane-1-carbonyl]-L-alanyl}-N²-{[(1S)-1,3-dicarboxypropyl]carbamoyl}-L-lysine (2.6 μg) dissolved in 152 μL 400 mM sodium acetate buffer (pH 5.6) containing 0.5 mg/mL pABA was mixed with thorium-227 in 0.5M HCl (2 μL) at 0.375 MBq/nmol specific activity and RAC of 6.0 MBq/mL at 90° C. for 40 min. The labelling efficiency was determined to be 90% by iTLC.

#18 Linker

Intermediate 78

### methyl N-(tert-butoxycarbonyl)-4-cyclobutyl-L-phenylalaninate

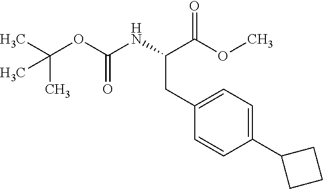 [SMILES: COC(=O)[C@H](Cc1ccc(C2CCC2)cc1)NC(=O)OC(C)(C)C]

Methyl 4-bromo-N-(tert-butoxycarbonyl)-L-phenylalaninate (853 mg, 2.38 mmol), and Ir(4′,6′-dF-5-CF3-ppy)2(4,4′-dtbbpy)PF6 (53.5 mg, 47.6 μmol) were dissolved in a reaction vial in (trifluoromethyl)benzene (51 ml). In a separate vial, 1,2-dimethoxyethane-dichloronickel (1:1) (2.62 mg, 11.9 μmol; CAS-RN:[29046-78-4]) and 4,4′-di-tert-butyl-2,2′-bipyridine (3.20 mg, 11.9 μmol; CAS-RN:[72914-19-3]) were stirred in N,N-dimethylacetamide (26 ml) for 5 min. The catalyst solution was added to the sealed reaction vial. The mixture was degassed by sparging for 20 min. Then bromocyclobutane (1.0 ml, 11 mmol; CAS-RN:[4399-47-7]), 2,6-Lutidine (1.7 ml, 14 mmol; CAS-RN:[108-48-5]) and 1,1,1,3,3,3-hexamethyl-2-(trimethylsilyl)trisilane (730 μl, 2.4 mmol; CAS-RN:[1873-77-4]) were added. The vial was stirred in a water bath and irradiated by two 40 W Kessil LED Aquarium lamps (A160WE tuna blue). The mixture was quenched with half sat. sodium hydrogen carbonate and extracted 3 times with EtOAc. The combined organic layers were dried and concentrated under reduced pressure. The residue was purified by flash chromatography (SiO2, hexane/EtOAc gradient 0%-25%) to give 377 mg (47% yield) of the target compound.

¹H-NMR (400 MHz, DMSO-d6) δ[ppm]: 1.145 (0.99), 1.163 (2.06), 1.180 (1.05), 1.235 (1.09), 1.306 (16.00), 1.921 (0.44), 1.944 (0.50), 1.970 (0.40), 1.978 (3.74), 2.010 (0.58), 2.017 (0.52), 2.032 (0.62), 2.038 (0.76), 2.062 (0.47), 2.225 (0.69), 2.232 (0.84), 2.238 (0.45), 2.245 (0.48), 2.252 (0.81), 2.259 (0.51), 2.508 (0.81), 2.513 (0.53), 2.790 (0.43), 2.815 (0.42), 2.908 (0.43), 2.921 (0.45), 3.452 (0.53), 3.596 (6.20), 4.007 (0.83), 4.025 (0.82), 7.127 (6.03), 7.256 (0.67), 7.277 (0.61).

Intermediate 79

### N-(tert-butoxycarbonyl)-4-cyclobutyl-L-phenylalanine

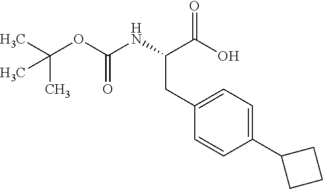 [SMILES: CC(C)(C)OC(=O)N[C@@H](Cc1ccc(C2CCC2)cc1)C(=O)O]

methyl N-(tert-butoxycarbonyl)-4-cyclobutyl-L-phenylalaninate (733 mg, 2.20 mmol) was solubilised in THF (18 ml), lithium hydroxide (11 ml, 1.0 M, 11 mmol) was added and the mixture was stirred overnight at rt. The mixture was acidified with HCl (1.0 M) and extracted with DCM. The organic layer was dried, evaporated and purified by flash chromatography (SiO2, DCM/Ethanol gradient 0%-20%) to give 530 mg (94% purity, 71% yield) of the target compound.

LC-MS (Method 1): Rt=1.31 min; MS (ESIpos): m/z=320 [M+H]⁺

¹H-NMR (400 MHz, DMSO-d6) δ[ppm]: 1.247 (1.12), 1.314 (16.00), 1.931 (0.42), 1.953 (0.50), 2.023 (0.58), 2.029 (0.51), 2.045 (0.62), 2.050 (0.73), 2.074 (0.47), 2.235 (0.67), 2.241 (0.82), 2.247 (0.43), 2.254 (0.46), 2.261 (0.80), 2.268 (0.48), 2.518 (0.46), 2.948 (0.40), 3.459 (0.49), 5.758 (3.16), 7.057 (0.61), 7.078 (0.59), 7.114 (0.65), 7.135 (2.37), 7.147 (2.41), 7.167 (0.64).

Intermediate 80

### tri-tert-butyl (6S,13S,17S)-6-[(4-cyclobutylphenyl)methyl]-2,2-dimethyl-4,7,15-trioxo-3-oxa-5,8,14,16-tetraazanonadecane-13,17,19-tricarboxylate

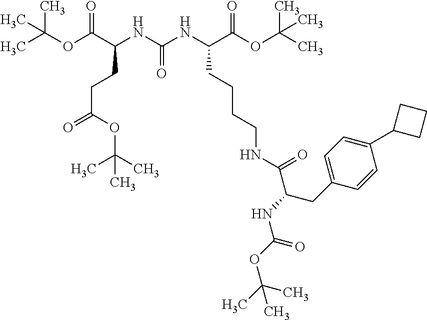 [SMILES: CC(C)(C)OC(=O)CC[C@H](NC(=O)N[C@@H](CCCCNC(=O)[C@H](Cc1ccc(C2CCC2)cc1)NC(=O)OC(C)(C)C)C(=O)OC(C)(C)C)C(=O)OC(C)(C)C]

di-tert-butyl N-{[(2S)-6-amino-1-tert-butoxy-1-oxohexan-2-yl]carbamoyl}-L-glutamate (779 mg, 1.60 mmol) and N-(tert-butoxycarbonyl)-4-cyclobutyl-L-phenylalanine (510 mg, 1.60 mmol) were solubilised in DMF (12 ml), N,N-diisopropylethylamine (1.0 ml, 5.7 mmol) and T₃P (2.1 ml, 50% purity in DMF, 3.5 mmol) were added and the mixture was stirred under argon at rt. The mixture was evaporated and purified by preparative H PLC (C18, acetonitrile/water with 0.1% formic acid) to give 370 mg (96% purity, 28% yield) of the target compound.

LC-MS (Method 1): Rt=1.65 min; MS (ESIpos): m/z=790 [M+H]⁺

Intermediate 81

### N⁶-(4-cyclobutyl-L-phenylalanyl)-N²-{[(1S)-1,3-dicarboxypropyl]carbamoyl}-L-lysine

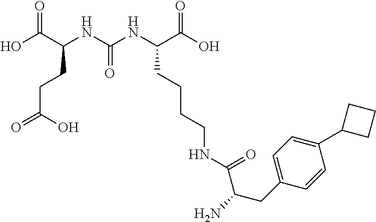 [SMILES: N[C@@H](Cc1ccc(C2CCC2)cc1)C(=O)NCCCC[C@H](NC(=O)N[C@@H](CCC(=O)O)C(=O)O)C(=O)O]

tri-tert-butyl (6S,13S,17S)-6-[(4-cyclobutylphenyl)methyl]-2,2-dimethyl-4,7,15-trioxo-3-oxa-5,8,14,16-tetraazanonadecane-13,17,19-tricarboxylate (370 mg, 469 μmol) was solubilised in DCM (6.0 ml), TFA (5.4 ml, 70 mmol) was added and the mixture was stirred under argon for 2 h at rt. The mixture was evaporated and purified by preparative HPLC (C18, acetonitrile/water with 0.1% formic acid) to give 115 mg (62% purity, 29% yield) of the target compound.

LC-MS (Method 1): Rt=0.72 min; MS (ESIpos): m/z=521 [M+H]⁺

Intermediate 82

### N⁶-(4-cyclobutyl-N-{(1r,4S)-4-[({[(9H-fluoren-9-yl)methoxy]carbonyl}amino)methyl]cyclohexane-1-carbonyl}-L-phenylalanyl)-N²-{[(1S)-1,3-dicarboxypropyl]carbamoyl}-L-lysine

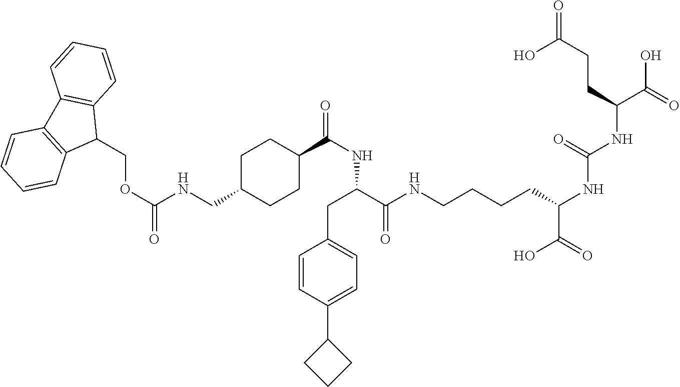 [SMILES: O=C(O)CC[C@H](NC(=O)N[C@@H](CCCCNC(=O)[C@H](Cc1ccc(C2CCC2)cc1)NC(=O)[C@H]1CC[C@H](CNC(=O)OCC2c3ccccc3-c3ccccc32)CC1)C(=O)O)C(=O)O]

N6-(4-cyclobutyl-L-phenylalanyl)-N2-{[(1S)-1,3-dicarboxypropyl]carbamoyl}-L-lysine (115 mg, 221 μmol) and (1r,4r)-4-[({[(9H-fluoren-9-yl)methoxy]carbonyl}amino)methyl]cyclohexane-1-carboxylic acid (88.0 mg, 232 μmol) were solubilised in DMF (2.5 ml), 4-methylmorpholine (73 μl, 660 μmol, CAS-RN: 109-02-4) and HATU (88.2 mg, 232 μmol) were added and the mixture was stirred under argon overnight at rt. The mixture was evaporated and purified by preparative HPLC (C18, acetonitrile/water with 0.1% formic acid) to give 68.0 mg (50% purity, 17% yield) of the target compound.

LC-MS (Method 1): Rt=1.29 min; MS (ESIpos): m/z=882 [M+H]⁺

### Example 18-A

### N⁶—{N-[(1r,4S)-4-(aminomethyl)cyclohexane-1-carbonyl]-4-cyclobutyl-L-phenylalanyl}-N²-{[(1S)-1,3-dicarboxypropyl]carbamoyl}-L-lysine

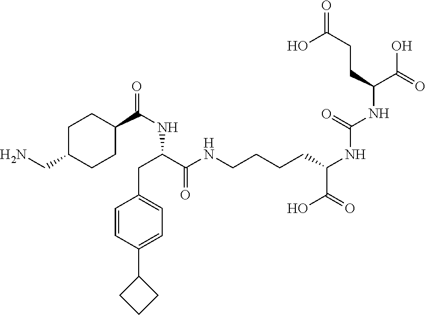 [SMILES: NC[C@H]1CC[C@H](C(=O)N[C@@H](Cc2ccc(C3CCC3)cc2)C(=O)NCCCC[C@H](NC(=O)N[C@@H](CCC(=O)O)C(=O)O)C(=O)O)CC1]

N6-(4-cyclobutyl-N-{(1r,4S)-4-[({[(9H-fluoren-9-yl)methoxy]carbonyl}amino)methyl]cyclohexane-1-carbonyl}-L-phenylalanyl)-N2-{[(1S)-1,3-dicarboxypropyl]carbamoyl}-L-lysine (68.0 mg, 77.1 μmol) was solubilised in DMF (1.2 ml), piperidine (150 μl, 1.5 mmol) was added and the mixture was stirred under argon overnight at rt. The mixture was evaporated and purified by preparative HPLC (C18, acetonitrile/water with 0.1% formic acid) to give 7.00 mg (95% purity, 13% yield) of the target compound.

LC-MS (Method 1): Rt=0.80 min; MS (ESIpos): m/z=661 [M+H]⁺

¹H-NMR (400 MHz, DMSO-d6) δ[ppm]: 0.867 (3.91), 0.898 (3.76), 1.137 (3.25), 1.168 (5.12), 1.232 (14.34), 1.322 (5.29), 1.450 (4.33), 1.504 (3.49), 1.540 (3.23), 1.572 (2.63), 1.674 (6.31), 1.767 (5.97), 1.785 (6.52), 1.898 (1.59), 1.919 (2.78), 1.943 (3.15), 1.967 (2.57), 1.983 (3.12), 2.005 (4.48), 2.028 (5.65), 2.051 (5.04), 2.078 (3.65), 2.171 (3.02), 2.208 (5.33), 2.228 (8.29), 2.249 (6.97), 2.328 (1.70), 2.598 (5.53), 2.669 (1.76), 2.695 (1.93), 2.727 (2.81), 2.753 (2.40), 2.891 (3.36), 2.914 (3.23), 2.927 (3.42), 2.961 (3.17), 3.043 (3.25), 3.420 (4.91), 3.442 (5.87), 3.463 (4.78), 3.556 (3.12), 3.596 (2.74), 3.937 (5.48), 4.373 (2.87), 4.386 (2.85), 6.187 (2.12), 6.385 (2.66), 6.401 (2.55), 7.076 (4.82), 7.096 (15.98), 7.107 (16.00), 7.127 (4.51), 7.874 (3.48), 8.034 (2.93), 8.054 (2.83), 8.288 (1.15).

#19 Linker

Intermediate 83 tri-tert-butyl (5S,12S,16S)-5-[(3,4-dimethoxyphenyl)methyl]-1-(9H-fluoren-9-yl)-3,6,14-trioxo-2-oxa-4,7,13,15-tetraazaoctadecane-12,16,18-tricarboxylate

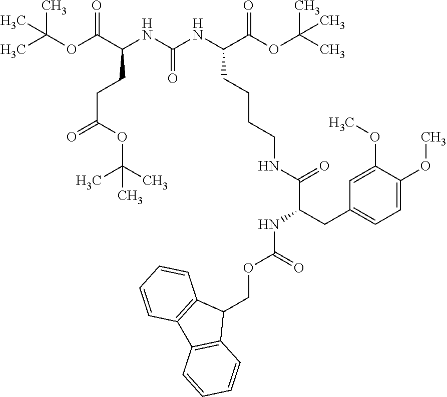 [SMILES: COc1ccc(C[C@H](NC(=O)OCC2c3ccccc3-c3ccccc32)C(=O)NCCCC[C@H](NC(=O)N[C@@H](CCC(=O)OC(C)(C)C)C(=O)OC(C)(C)C)C(=O)OC(C)(C)C)cc1OC]

di-tert-butyl N-{[(2S)-6-amino-1-tert-butoxy-1-oxohexan-2-yl]carbamoyl}-L-glutamate (270 mg, 554 μmol) and N-{[(9H-fluoren-9-yl)methoxy]carbonyl}-3-methoxy-O-methyl-L-tyrosine (248 mg, 554 μmol; CAS-RN:[184962-88-7]) were solubilised in DMF (4.3 ml), N,N-diisopropylethylamine (350 μl, 2.0 mmol) and T₃P (710 μl, 50% purity in DMF, 1.2 mmol) were added and the mixture was stirred under argon at rt. The mixture was evaporated and purified by preparative HPLC (C18, acetonitrile/water with 0.1% formic acid) to give 240 mg (91% purity, 43% yield) of the target compound.

LC-MS (Method 1): Rt=1.56 min; MS (ESIpos): m/z=918 [M+H]⁺

¹H-NMR (400 MHz, DMSO-d6) δ[ppm]: 1.368 (1.44), 1.380 (16.00), 1.383 (14.03), 2.518 (2.11), 2.523 (1.43), 2.673 (0.47), 3.670 (2.29), 3.709 (2.41), 4.142 (1.57), 6.794 (0.56), 6.904 (0.43), 6.908 (0.42), 7.399 (0.41), 7.401 (0.42), 7.636 (0.52), 7.655 (0.47), 7.867 (0.63), 7.886 (0.58).

Intermediate 84

### di-tert-butyl (2S)-2-({[(2S)-6-{[(2S)-2-amino-3-(3,4-dimethoxyphenyl)propanoyl]amino}-1-tert-butoxy-1-oxohexan-2-yl]carbamoyl}amino)pentanedioate

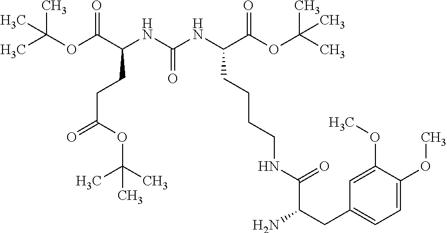 [SMILES: COc1ccc(C[C@H](N)C(=O)NCCCC[C@H](NC(=O)N[C@@H](CCC(=O)OC(C)(C)C)C(=O)OC(C)(C)C)C(=O)OC(C)(C)C)cc1OC]

tri-tert-butyl (5S,12S,16S)-5-[(3,4-dimethoxyphenyl)methyl]-1-(9H-fluoren-9-yl)-3,6,14-trioxo-2-oxa-4,7,13,15-tetraazaoctadecane-12,16,18-tricarboxylate (240 mg, 100% purity, 262 μmol) was solubilised in DMF (2.8 ml), piperidine (520 μl, 5.2 mmol) was added and the mixture was stirred under argon overnight at rt. The mixture was evaporated and purified by preparative HPLC (C18, acetonitrile/water with 0.1% formic acid) to give 100 mg (97% purity, 53% yield) of the target compound.

LC-MS (Method 1): Rt=1.48 min; MS (ESIpos): m/z=1083 [M+H]⁺

¹H-NMR (400 MHz, DMSO-d6) δ[ppm]: 1.239 (0.46), 1.378 (16.00), 1.386 (9.24), 2.297 (0.52), 2.518 (0.83), 2.522 (0.51), 4.293 (0.57), 7.320 (0.67), 7.322 (0.68), 7.338 (0.45), 7.341 (0.44), 7.410 (0.60), 7.684 (0.53), 7.702 (0.49), 7.879 (0.67), 7.898 (0.61), 8.978 (0.43), 8.982 (0.43).

Intermediate 85 tri-tert-butyl (3S,10S,14S)-3-[(3,4-dimethoxyphenyl)methyl]-1-{(1r,4S)-4-[({[(9H-fluoren-9-yl)methoxy]carbonyl}amino)methyl]cyclohexyl}-1,4,12-trioxo-2,5,11,13-tetraazahexadecane-10,14,16-tricarboxylate

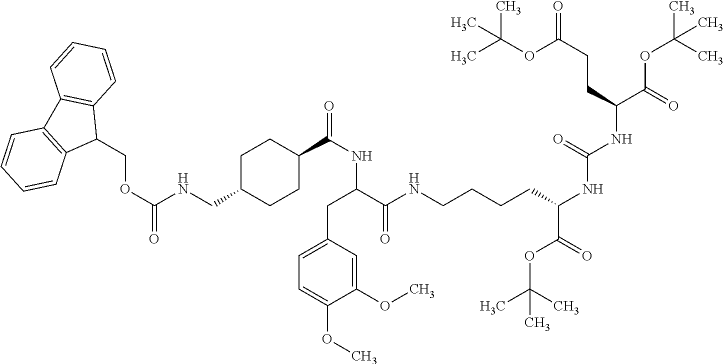 [SMILES: COc1ccc(CC(NC(=O)[C@H]2CC[C@H](CNC(=O)OCC3c4ccccc4-c4ccccc43)CC2)C(=O)NCCCC[C@H](NC(=O)N[C@@H](CCC(=O)OC(C)(C)C)C(=O)OC(C)(C)C)C(=O)OC(C)(C)C)cc1OC]

di-tert-butyl (2S)-2-({[(2S)-6-{[(2S)-2-amino-3-(3,4-dimethoxyphenyl)propanoyl]amino}-1-tert-butoxy-1-oxohexan-2-yl]carbamoyl}amino)pentanedioate (115 mg, 165 μmol) and (1r,4r)-4-[({[(9H-fluoren-9-yl)methoxy]carbonyl}amino)methyl]cyclohexane-1-carboxylic acid (94.2 mg, 248 μmol) were solubilised in DMF (1.3 ml), 4-methylmorpholine (55 μl, 500 μmol, CAS-RN: 109-02-4) and HATU (94.4 mg, 248 μmol) were added and the mixture was stirred under argon overnight at rt. The mixture was evaporated and purified by preparative HPLC (C18, acetonitrile/water with 0.1% formic acid) to give 48.0 mg (77% purity, 21% yield) of the target compound.

LC-MS (Method 1): Rₜ=1.56 min; MS (ESIpos): m/z=1057 [M+H]⁺

Intermediate 86

### tri-tert-butyl (3S,10S,14S)-1-[(1r,4S)-4-(aminomethyl)cyclohexyl]-3-[(3,4-dimethoxyphenyl)methyl]-1,4,12-trioxo-2,5,11,13-tetraazahexadecane-10,14,16-tricarboxylate

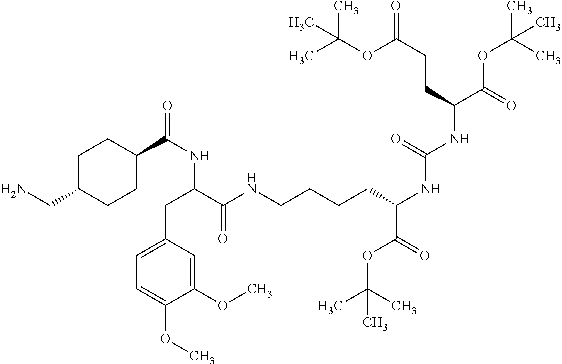 [SMILES: COc1ccc(CC(NC(=O)[C@H]2CC[C@H](CN)CC2)C(=O)NCCCC[C@H](NC(=O)N[C@@H](CCC(=O)OC(C)(C)C)C(=O)OC(C)(C)C)C(=O)OC(C)(C)C)cc1OC]

tri-tert-butyl (3S,10S,14S)-3-[(3,4-dimethoxyphenyl)methyl]-1-{(1r,4S)-4-[({[(9H-fluoren-9-yl)methoxy]carbonyl}amino)methyl]cyclohexyl}-1,4,12-trioxo-2,5,11,13-tetraazahexadecane-10,14,16-tricarboxylate (47.0 mg, 44.5 μmol) was solubilised in DMF (680 μl), piperidine (88 μl, 890 μmol) was added and the mixture was stirred under argon overnight at rt. The mixture was evaporated and purified by preparative HPLC (C18, acetonitrile/water with 0.1% formic acid) to give 14.0 mg (90% purity, 34% yield) of the target compound.

LC-MS (Method 1): Rₜ=1.15 min; MS (ESIpos): m/z=835 [M+H]⁺

¹H-NMR (400 MHz, DMSO-d6) δ[ppm]: 1.230 (0.58), 1.250 (0.45), 1.383 (16.00), 1.388 (12.74), 1.391 (13.44), 2.451 (0.48), 2.468 (0.74), 2.518 (4.00), 2.523 (2.70), 3.689 (3.86), 3.710 (3.69), 6.796 (0.71), 6.817 (0.65), 6.822 (0.60), 6.827 (0.51).

### Example 19-A

### N⁶—{N-[(1r,4S)-4-(aminomethyl)cyclohexane-1-carbonyl]-3-methoxy-O-methyl-L-tyrosyl}-N²-{[(1S)-1,3-dicarboxypropyl]carbamoyl}-L-lysine

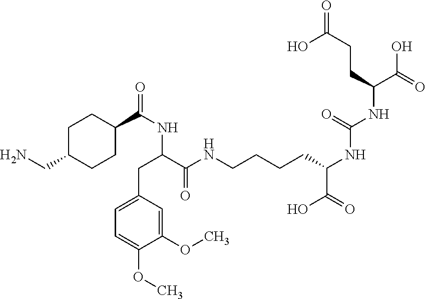 [SMILES: COc1ccc(CC(NC(=O)[C@H]2CC[C@H](CN)CC2)C(=O)NCCCC[C@H](NC(=O)N[C@@H](CCC(=O)O)C(=O)O)C(=O)O)cc1OC]

tri-tert-butyl (3S,10S,14S)-1-[(1r,4S)-4-(aminomethyl)cyclohexyl]-3-[(3,4-dimethoxyphenyl)methyl]-1,4,12-trioxo-2,5,11,13-tetraazahexadecane-10,14,16-tricarboxylate (14.0 mg, 16.8 μmol) was solubilised in DCM (650 μl), TFA (520 μl, 6.7 mmol) was added and the mixture was stirred under argon overnight at rt. The mixture was evaporated and purified by preparative HPLC (C18, acetonitrile/water with 0.1% formic acid) to give 3.00 mg (95% purity, 26% yield) of the target compound.

LC-MS (Method 1): Rₜ=0.59 min; MS (ESIpos): m/z=667 [M+H]⁺

¹H-NMR (400 MHz, DMSO-d6) δ[ppm]: 0.877 (1.06), 0.905 (1.15), 1.139 (0.73), 1.172 (0.90), 1.243 (2.08), 1.341 (1.63), 1.456 (1.29), 1.519 (1.01), 1.552 (1.07), 1.602 (0.78), 1.678 (1.82), 1.748 (1.06), 1.769 (1.23), 2.072 (1.04), 2.094 (0.95), 2.127 (0.67), 2.145 (0.67), 2.184 (0.98), 2.210 (1.09), 2.229 (0.96), 2.326 (1.12), 2.331 (0.92), 2.350 (0.98), 2.539 (0.81), 2.603 (1.69), 2.668 (1.65), 2.691 (1.18), 2.700 (1.12), 2.727 (2.60), 2.851 (1.24), 2.861 (1.38), 2.887 (3.00), 2.964 (1.41), 3.064 (1.83), 3.082 (1.80), 3.098 (1.71), 3.164 (2.16), 3.418 (8.19), 3.686 (16.00), 3.706 (14.18), 3.863 (0.87), 3.943 (2.08), 4.336 (0.62), 4.357 (1.03), 4.370 (1.03), 6.185 (0.62), 6.403 (0.72), 6.695 (1.23), 6.716 (1.69), 6.795 (2.57), 6.815 (1.88), 6.836 (2.88), 7.885 (1.12), 8.035 (0.95), 8.055 (0.93).

#20 Linker

Intermediate 87

### tri-tert-butyl (5S,12S,16S)-1-(9H-fluoren-9-yl)-3,6,14-trioxo-5-{3-[(pyrazine-2-carbonyl)amino]propyl}-2-oxa-4,7,13,15-tetraazaoctadecane-12,16,18-tricarboxylate

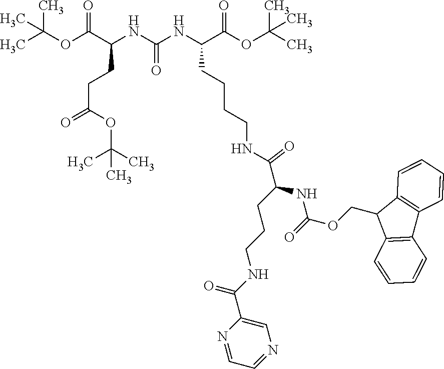 [SMILES: CC(C)(C)OC(=O)CC[C@H](NC(=O)N[C@@H](CCCCNC(=O)[C@H](CCCNC(=O)c1cnccn1)NC(=O)OCC1c2ccccc2-c2ccccc21)C(=O)OC(C)(C)C)C(=O)OC(C)(C)C]

di-tert-butyl N-{[(2S)-6-amino-1-tert-butoxy-1-oxohexan-2-yl]carbamoyl}-L-glutamate (287 mg, 97% purity, 571 μmol) and N2-{[(9H-fluoren-9-yl)methoxy]carbonyl}-N5-(pyrazine-2-carbonyl)-L-ornithine (394 mg, 856 μmol) were solubilised in DMF (4.4 ml), 4-methylmorpholine (190 μl, 1.7 mmol, CAS-RN: 109-02-4) and HATU (326 mg, 856 μmol) were added and the mixture was stirred under argon overnight at rt. The mixture was evaporated and purified by preparative HPLC (C18, acetonitrile/water with 0.1% formic acid) to give 411 mg (95% purity, 74% yield) of the target compound.

LC-MS (Method 1): Rₜ=1.41 min; MS (ESIpos): m/z=931 [M+H]⁺

¹H-NMR (500 MHz, DMSO-d6) δ[ppm]: 1.368 (5.96), 1.377 (16.00), 2.298 (0.53), 2.515 (0.71), 2.518 (0.62), 2.522 (0.47), 4.206 (0.49), 6.282 (0.44), 7.310 (0.56), 7.388 (0.52), 7.402 (0.50), 7.873 (0.79), 7.888 (0.68), 8.721 (0.43), 8.723 (0.46), 8.726 (0.41), 8.860 (0.73), 8.865 (0.68), 9.177 (0.53), 9.179 (0.55).

Intermediate 88

### tri-tert-butyl (6S,13S,17S)-6-amino-1,7,15-trioxo-1-(pyrazin-2-yl)-2,8,14,16-tetraazanonadecane-13,17,19-tricarboxylate

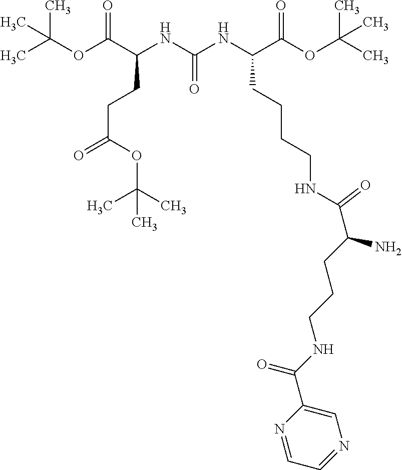 [SMILES: CC(C)(C)OC(=O)CC[C@H](NC(=O)N[C@@H](CCCCNC(=O)[C@@H](N)CCCNC(=O)c1cnccn1)C(=O)OC(C)(C)C)C(=O)OC(C)(C)C]

tri-tert-butyl (5S,12S,16S)-1-(9H-fluoren-9-yl)-3,6,14-trioxo-5-{3-[(pyrazine-2-carbonyl)amino]propyl}-2-oxa-4,7,13,15-tetraazaoctadecane-12,16,18-tricarboxylate (405 mg, 94% purity, 409 μmol) was solubilised in DMF (4.4 ml), piperidine (810 μl, 8.2 mmol) was added and the mixture was stirred under argon overnight at rt. The mixture was evaporated and purified by preparative HPLC (C18, acetonitrile/water with 0.1% formic acid) to give 285 mg (94% purity, 92% yield) of the target compound.

LC-MS (Method 1): Rₜ=0.94 min; MS (ESIpos): m/z=709 [M+H]⁺

Intermediate 89

### tri-tert-butyl (6S,13S,17S)-6-({(1r,4S)-4-[({[(9H-fluoren-9-yl)methoxy]carbonyl}amino)methyl]cyclohexane-1-carbonyl}amino)-1,7,15-trioxo-1-(pyrazin-2-yl)-2,8,14,16-tetraazanonadecane-13,17,19-tricarboxylate

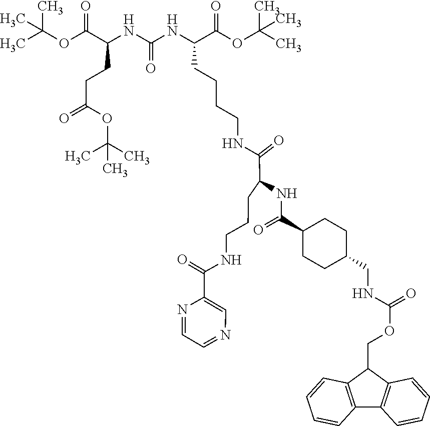 [SMILES: CC(C)(C)OC(=O)CC[C@H](NC(=O)N[C@@H](CCCCNC(=O)[C@H](CCCNC(=O)c1cnccn1)NC(=O)[C@H]1CC[C@H](CNC(=O)OCC2c3ccccc3-c3ccccc32)CC1)C(=O)OC(C)(C)C)C(=O)OC(C)(C)C]

tri-tert-butyl (6S,13S,17S)-6-amino-1,7,15-trioxo-1-(pyrazin-2-yl)-2,8,14,16-tetraazanonadecane-13,17,19-tricarboxylate (285 mg, 93% purity, 374 μmol) and (1r,4r)-4-[({[(9H-fluoren-9-yl)methoxy]carbonyl}amino)methyl]cyclohexane-1-carboxylic acid (213 mg, 562 μmol) were solubilised in DMF (2.9 ml), 4-methylmorpholine (120 μl, 1.1 mmol, CAS-RN: 109-02-4) and HATU (214 mg, 562 μmol) were added and the mixture was stirred under argon overnight at rt. The mixture was evaporated and purified by preparative HPLC (C18, acetonitrile/water with 0.1% formic acid) to give 72.0 mg (46% purity, 8% yield) and 63.0 mg (66% purity, 10% yield) of the target compound.

LC-MS (Method 1): Rₜ=1.48 min; MS (ESIpos): m/z=1070 [M+H]⁺

Intermediate 90

### tri-tert-butyl (6S,13S,17S)-6-{[(1r,4S)-4-(aminomethyl)cyclohexane-1-carbonyl]amino}-1,7,15-trioxo-1-(pyrazin-2-yl)-2,8,14,16-tetraazanonadecane-13,17,19-tricarboxylate

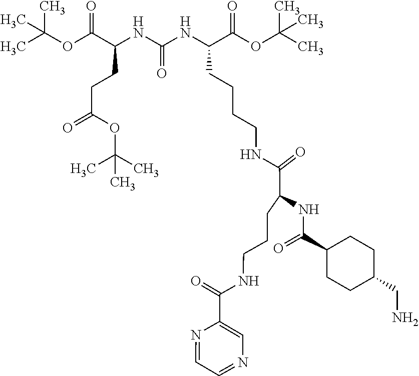 [SMILES: CC(C)(C)OC(=O)CC[C@H](NC(=O)N[C@@H](CCCCNC(=O)[C@H](CCCNC(=O)c1cnccn1)NC(=O)[C@H]1CC[C@H](CN)CC1)C(=O)OC(C)(C)C)C(=O)OC(C)(C)C]

tri-tert-butyl (6S,13S,17S)-6-({(1r,4S)-4-[({[(9H-fluoren-9-yl)methoxy]carbonyl}amino)methyl]cyclohexane-1-carbonyl}amino)-1,7,15-trioxo-1-(pyrazin-2-yl)-2,8,14,16-tetraazanonadecane-13,17,19-tricarboxylate (63.0 mg, 66% purity, 38.9 μmol) was solubilised in DMF (1.0 ml), piperidine (77 μl, 780 μmol) was added and the mixture was stirred under argon overnight at rt. The mixture was evaporated and purified by preparative HPLC (C18, acetonitrile/water with 0.1% formic acid) to give 19.0 mg (37% purity, 21% yield) and 23.0 mg (82% purity, 57% yield) of the target compound.

LC-MS (Method 1): Rt=1.02 min; MS (ESIpos): m/z=848 [M+H]⁺

### Example 20-A

### N⁶—{N²-[(1r,4S)-4-(aminomethyl)cyclohexane-1-carbonyl]-N⁵-(pyrazine-2-carbonyl)-L-ornithyl}-N²-{[(1S)-1,3-dicarboxypropyl]carbamoyl}-L-lysine

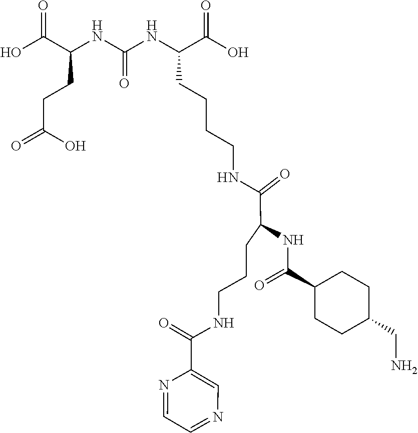 [SMILES: NC[C@H]1CC[C@H](C(=O)N[C@@H](CCCNC(=O)c2cnccn2)C(=O)NCCCC[C@H](NC(=O)N[C@@H](CCC(=O)O)C(=O)O)C(=O)O)CC1]

tri-tert-butyl (6S,13S,17S)-6-{[(1r,4S)-4-(aminomethyl)cyclohexane-1-carbonyl]amino}-1,7,15-trioxo-1-(pyrazin-2-yl)-2,8,14,16-tetraazanonadecane-13,17,19-tricarboxylate (23.0 mg, 82% purity, 22.3 μmol) was solubilised in DCM (1.4 ml), TFA (1.4 ml, 18 mmol) was added and the mixture was stirred under argon at rt. The mixture was evaporated and purified by preparative HPLC (C18, acetonitrile/water with 0.1% formic acid) to give 9.00 mg (93% purity, 55% yield) of the target compound.

LC-MS (Method 1): Rₜ=0.49 min; MS (ESIpos): m/z=680 [M+H]⁺

¹H-NMR (400 MHz, DMSO-d6) δ[ppm]: 0.901 (3.70), 0.931 (4.06), 1.230 (8.78), 1.245 (8.24), 1.263 (9.01), 1.318 (5.37), 1.337 (6.93), 1.354 (6.93), 1.486 (9.91), 1.545 (4.96), 1.562 (4.66), 1.583 (4.18), 1.621 (4.36), 1.751 (14.51), 1.904 (0.96), 2.072 (0.66), 2.083 (0.72), 2.122 (2.15), 2.151 (3.70), 2.194 (6.27), 2.213 (8.78), 2.322 (2.87), 2.326 (3.64), 2.331 (2.75), 2.522 (9.79), 2.539 (13.73), 2.632 (6.69), 2.664 (4.54), 2.668 (4.90), 2.673 (3.82), 2.991 (6.99), 3.004 (8.54), 3.020 (7.16), 3.050 (4.72), 3.164 (4.78), 3.277 (13.85), 3.290 (14.27), 3.384 (9.73), 3.502 (10.27), 3.965 (6.69), 3.979 (7.82), 3.997 (7.82), 4.014 (6.33), 4.164 (5.31), 4.176 (5.07), 4.275 (1.61), 4.515 (1.01), 6.223 (3.34), 6.241 (3.22), 6.345 (4.42), 6.365 (4.18), 7.340 (0.54), 7.815 (5.55), 7.848 (5.31), 7.868 (4.96), 7.984 (0.48), 8.202 (5.01), 8.720 (10.63), 8.855 (11.94), 8.860 (10.93), 8.949 (3.52), 8.964 (6.99), 8.979 (3.52), 9.166 (15.88), 9.169 (16.00), 9.310 (0.60).

#21 Linker

Intermediate 91

### tri-tert-butyl (3S,10S,14S)-3-[(naphthalen-2-yl)methyl]-4,12-dioxo-1-(6-oxo-1,6-dihydropyridin-2-yl)-2,5,11,13-tetraazahexadecane-10,14,16-tricarboxylate

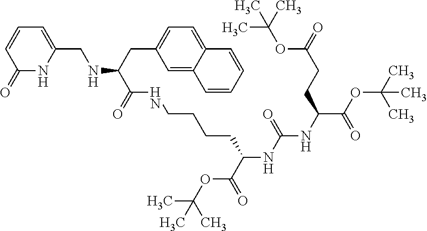 [SMILES: CC(C)(C)OC(=O)CC[C@H](NC(=O)N[C@@H](CCCCNC(=O)[C@H](Cc1ccc2ccccc2c1)NCc1cccc(=O)[nH]1)C(=O)OC(C)(C)C)C(=O)OC(C)(C)C]

di-tert-butyl (2S)-2-({[(2S)-6-{[(2S)-2-amino-3-(naphthalen-2-yl)propanoyl]amino}-1-tert-butoxy-1-oxohexan-2-yl]carbamoyl}amino)pentanedioate (403 mg, 25% purity, 147 μmol) and 6-oxo-1,6-dihydropyridine-2-carbaldehyde (19.8 mg, 96% purity, 154 μmol; CAS-RN:[358751-77-6]) were solubilised in MeOH (1.2 ml), acetic acid (8.4 μl, 150 μmol) was added and sodium cyanoborohydride (27.7 mg, 441 μmol) was added carefully. The mixture was stirred for 45 min at rt. The mixture was evaporated, diluted with DMF and purified by preparative HPLC (C18, acetonitrile/water with 0.1% formic acid) to give 72.0 mg (90% purity, 56% yield) of the target compound.

LC-MS (Method 1): Rₜ=135.00 min; MS (ESIpos): m/z=793 [M+H]⁺

¹H-NMR (400 MHz, DMSO-d6) δ[ppm]: 1.382 (16.00), 2.518 (0.99), 2.522 (0.61), 7.795 (0.43), 7.816 (0.44).

Intermediate 92

### tri-tert-butyl (3S,10S,14S)-1-{(1r,4S)-4-[({[(9H-fluoren-9-yl)methoxy]carbonyl}amino)methyl]cyclohexyl}-3-[(naphthalen-2-yl)methyl]-1,4,12-trioxo-2-[(6-oxo-1,6-dihydropyridin-2-yl)methyl]-2,5,11,13-tetraazahexadecane-10,14,16-tricarboxylate

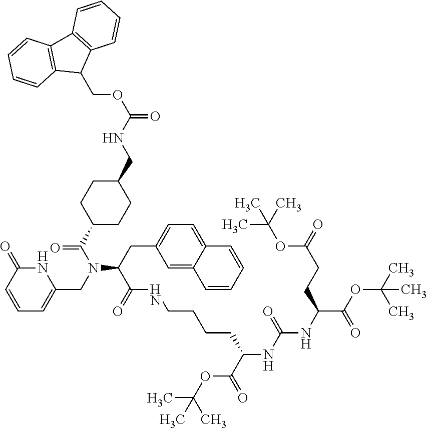 [SMILES: CC(C)(C)OC(=O)CC[C@H](NC(=O)N[C@@H](CCCCNC(=O)[C@H](Cc1ccc2ccccc2c1)N(Cc1cccc(=O)[nH]1)C(=O)[C@H]1CC[C@H](CNC(=O)OCC2c3ccccc3-c3ccccc32)CC1)C(=O)OC(C)(C)C)C(=O)OC(C)(C)C]

tri-tert-butyl (3S,10S,14S)-3-[(naphthalen-2-yl)methyl]-4,12-dioxo-1-(6-oxo-1,6-dihydropyridin-2-yl)-2,5,11,13-tetraazahexadecane-10,14,16-tricarboxylate (65.0 mg, 82.1 μmol) and (1r,4r)-4-[({[(9H-fluoren-9-yl)methoxy]carbonyl}amino)methyl]cyclohexane-1-carboxylic acid (46.7 mg, 123 μmol) were solubilised in DMF (630 μl), 4-methylmorpholine (27 μl, 250 μmol, CAS-RN: 109-02-4) and HATU (46.8 mg, 123 μmol) were added and the mixture was stirred under argon at rt. The mixture was evaporated and purified by preparative HPLC (C18, acetonitrile/water with 0.1% formic acid) to give 10.0 mg (11% yield) of the target compound.

LC-MS (Method 1): Rt=1.62 min; MS (ESIpos): m/z=1155 [M+H]⁺

Intermediate 93

### tri-tert-butyl (3S,10S,14S)-1-[(1r,4S)-4-(aminomethyl)cyclohexyl]-3-[(naphthalen-2-yl)methyl]-1,4,12-trioxo-2-[(6-oxo-1,6-dihydropyridin-2-yl)methyl]-2,5,11,13-tetraazahexadecane-10,14,16-tricarboxylate

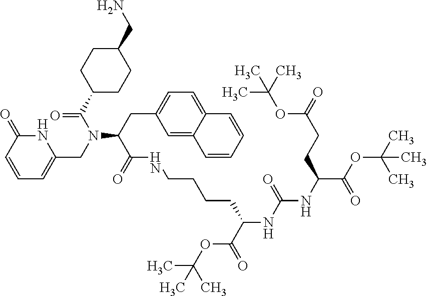 [SMILES: CC(C)(C)OC(=O)CC[C@H](NC(=O)N[C@@H](CCCCNC(=O)[C@H](Cc1ccc2ccccc2c1)N(Cc1cccc(=O)[nH]1)C(=O)[C@H]1CC[C@H](CN)CC1)C(=O)OC(C)(C)C)C(=O)OC(C)(C)C]

tri-tert-butyl (3S,10S,14S)-1-{(1r,4S)-4-[({[(9H-fluoren-9-yl)methoxy]carbonyl}amino)methyl]cyclohexyl}-3-[(naphthalen-2-yl)methyl]-1,4,12-trioxo-2-[(6-oxo-1,6-dihydropyridin-2-yl)methyl]-2,5,11,13-tetraazahexadecane-10,14,16-tricarboxylate (41.0 mg, 90% purity, 32.0 μmol) was solubilised in DMF (840 μl), piperidine (63 μl, 640 μmol) was added and the mixture was stirred under argon at rt. The mixture was evaporated and purified by preparative HPLC (C18, acetonitrile/water with 0.1% formic acid) to give 5.00 mg (85% purity, 14% yield) of the target compound.

LC-MS (Method 1): Rₜ=1.22 min; MS (ESIneg): m/z=930 [M−H]⁻

### Example 21-A

### (3S,10S,14S)-1-[(1r,4S)-4-(aminomethyl)cyclohexyl]-3-[(naphthalen-2-yl)methyl]-1,4,12-trioxo-2-[(6-oxo-1,6-dihydropyridin-2-yl)methyl]-2,5,11,13-tetraazahexadecane-10,14,16-tricarboxylic acid

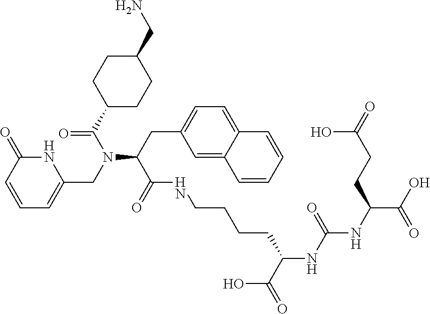 [SMILES: NC[C@H]1CC[C@H](C(=O)N(Cc2cccc(=O)[nH]2)[C@@H](Cc2ccc3ccccc3c2)C(=O)NCCCC[C@H](NC(=O)N[C@@H](CCC(=O)O)C(=O)O)C(=O)O)CC1]

tri-tert-butyl (3S,10S,14S)-1-[(1r,4S)-4-(aminomethyl)cyclohexyl]-3-[(naphthalen-2-yl)methyl]-1,4,12-trioxo-2-[(6-oxo-1,6-dihydropyridin-2-yl)methyl]-2,5,11,13-tetraazahexadecane-10,14,16-tricarboxylate (5.00 mg, 5.37 μmol) was solubilised in DCM (330 μl), TFA (330 μl, 4.3 mmol) was added and the mixture was stirred under argon at rt. The mixture was evaporated to give 1.50 mg (90% purity, 33% yield) of the target compound.

LC-MS (Method 1): Rₜ=0.75 min; MS (ESIpos): m/z=764 [M+H]⁺

¹H-NMR (400 MHz, DMSO-d6) δ[ppm]: 1.234 (13.49), 1.313 (8.46), 1.538 (6.40), 1.624 (8.69), 2.179 (5.26), 2.197 (5.71), 2.227 (5.94), 2.327 (14.86), 2.636 (5.26), 2.668 (16.00), 3.873 (4.80), 3.954 (6.63), 4.522 (5.03), 6.068 (5.49), 6.090 (5.71), 7.207 (4.80), 7.431 (6.63), 7.464 (11.66), 7.476 (11.89), 7.667 (9.37), 7.784 (8.46), 7.836 (9.83), 7.856 (12.80).

#22 Linker

Intermediate 94

### tri-tert-butyl (5S,12S,16S)-1-(9H-fluoren-9-yl)-3,6,14-trioxo-5-[(quinolin-3-yl)methyl]-2-oxa-4,7,13,15-tetraazaoctadecane-12,16,18-tricarboxylate

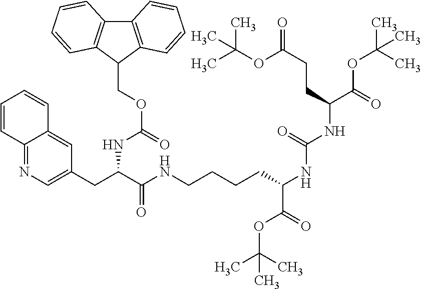 [SMILES: CC(C)(C)OC(=O)CC[C@H](NC(=O)N[C@@H](CCCCNC(=O)[C@H](Cc1cnc2ccccc2c1)NC(=O)OCC1c2ccccc2-c2ccccc21)C(=O)OC(C)(C)C)C(=O)OC(C)(C)C]

di-tert-butyl N-{[(2S)-6-amino-1-tert-butoxy-1-oxohexan-2-yl]carbamoyl}-L-glutamate (1.33 g, 2.74 mmol) and N-{[(9H-fluoren-9-yl)methoxy]carbonyl}-3-quinolin-3-yl-L-alanine (1.20 g, 2.74 mmol; CAS-RN:[281655-61-6]) were solubilised in DMF (21 ml), 4-methylmorpholine (900 μl, 8.2 mmol, CAS-RN: 109-02-4) and HATU (1.25 g, 3.28 mmol) were added and the mixture was stirred under argon overnight at rt. The mixture was diluted with water and extracted with DCM. The organic phase was washed with brine, dried and evaporated. The residue was by y flash chromatography (SiO2, hexane/EtOAc gradient 0%-100%) and preparative HPLC (C18, acetonitrile/water with 0.1% formic acid) to give 680 mg (100% purity, 27% yield) of the target compound.

LC-MS (Method 1): Rₜ=1.56 min; MS (ESIpos): m/z=909 [M+H]⁺

¹H-NMR (400 MHz, DMSO-d6) δ[ppm]: 1.357 (16.00), 2.517 (0.67), 2.522 (0.45), 4.083 (1.34), 7.349 (0.56), 7.368 (0.45), 7.527 (0.45), 7.546 (0.56), 7.821 (0.90), 7.840 (0.78), 8.821 (0.67), 8.826 (0.56).

Intermediate 95

### di-tert-butyl (2S)-2-({[(2S)-6-{[(2S)-2-amino-3-(quinolin-3-yl)propanoyl]amino}-1-tert-butoxy-1-oxohexan-2-yl]carbamoyl}amino)pentanedioate

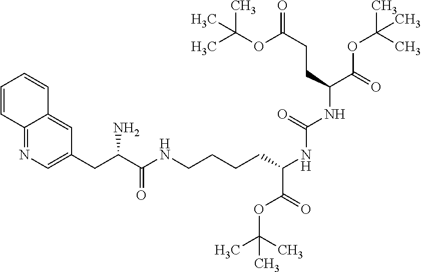 [SMILES: CC(C)(C)OC(=O)CC[C@H](NC(=O)N[C@@H](CCCCNC(=O)[C@@H](N)Cc1cnc2ccccc2c1)C(=O)OC(C)(C)C)C(=O)OC(C)(C)C]

tri-tert-butyl (5S,12S,16S)-1-(9H-fluoren-9-yl)-3,6,14-trioxo-5-[(quinolin-3-yl)methyl]-2-oxa-4,7,13,15-tetraazaoctadecane-12,16,18-tricarboxylate (680 mg, 749 μmol) was solubilised in DMF (5.8 ml), piperidine (1.5 ml, 15 mmol) was added and the mixture was stirred under argon for 3 h at rt. The mixture was evaporated and purified by preparative HPLC (C18, acetonitrile/water with 0.1% formic acid) to give 254 mg (100% purity, 49% yield) of the target compound.

LC-MS (Method 1): Rt=1.11 min; MS (ESIpos): m/z=687 [M+H]⁺

¹H-NMR (400 MHz, DMSO-d6) δ[ppm]: 1.363 (15.61), 1.366 (16.00), 1.895 (0.39), 2.517 (0.59), 2.522 (0.39), 8.094 (0.39), 8.098 (0.39), 8.237 (0.88), 8.728 (0.69), 8.734 (0.59).

Intermediate 96

### tri-tert-butyl (3S,10S,14S)-1-{(1r,4S)-4-[({[(9H-fluoren-9-yl)methoxy]carbonyl}amino)methyl]cyclohexyl}-1,4,12-trioxo-3-[(quinolin-3-yl)methyl]-2,5,11,13-tetraazahexadecane-10,14,16-tricarboxylate

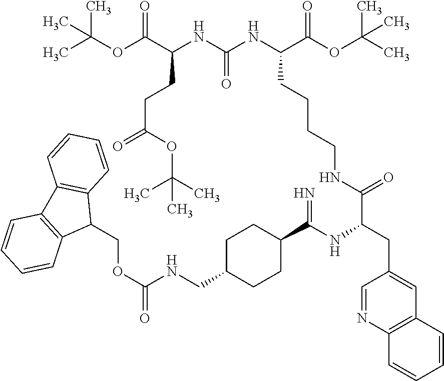 [SMILES: CC(C)(C)OC(=O)CC[C@H](NC(=O)N[C@@H](CCCCNC(=O)[C@H](Cc1cnc2ccccc2c1)NC(=N)[C@H]1CC[C@H](CNC(=O)OCC2c3ccccc3-c3ccccc32)CC1)C(=O)OC(C)(C)C)C(=O)OC(C)(C)C]

di-tert-butyl (2S)-2-({[(2S)-6-{[(2S)-2-amino-3-(quinolin-3-yl)propanoyl]amino}-1-tert-butoxy-1-oxohexan-2-yl]carbamoyl}amino)pentanedioate (90.0 mg, 131 μmol) and (1r,4r)-4-[({[(9H-fluoren-9-yl)methoxy]carbonyl}amino)methyl]cyclohexane-1-carboxylic acid (54.8 mg, 144 μmol) were solubilised in DMF (2.0 ml), 4-methylmorpholine (43 μl, 390 μmol, CAS-RN: 109-02-4) and HATU (54.9 mg, 144 μmol) were added and the mixture was stirred under argon overnight at rt. The mixture was evaporated and purified by preparative HPLC (C18, acetonitrile/water with 0.1% formic acid) to give 42.0 mg (100% purity, 31% yield) of the target compound.

LC-MS (Method 1): Rₜ=1.54 min; MS (ESIpos): m/z=1048 [M+H]⁺

Intermediate 97

### tri-tert-butyl (3S,10S,14S)-1-[(1r,4S)-4-(aminomethyl)cyclohexyl]-1,4,12-trioxo-3-[(quinolin-3-yl)methyl]-2,5,11,13-tetraazahexadecane-10,14,16-tricarboxylate

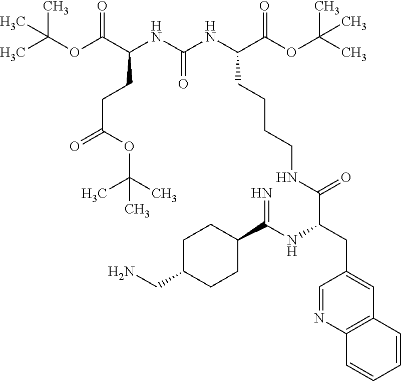 [SMILES: CC(C)(C)OC(=O)CC[C@H](NC(=O)N[C@@H](CCCCNC(=O)[C@H](Cc1cnc2ccccc2c1)NC(=N)[C@H]1CC[C@H](CN)CC1)C(=O)OC(C)(C)C)C(=O)OC(C)(C)C]

tri-tert-butyl (3S,10S,14S)-1-{(1r,4S)-4-[({[(9H-fluoren-9-yl)methoxy]carbonyl}amino)methyl]cyclohexyl}-1,4,12-trioxo-3-[(quinolin-3-yl)methyl]-2,5,11,13-tetraazahexadecane-10,14,16-tricarboxylate (35.0 mg, 33.4 μmol) was solubilised in DMF (510 μl), piperidine (66 μl, 670 μmol) was added and the mixture was stirred under argon for 2 h at rt.

The mixture was evaporated and purified by preparative HPLC (C18, acetonitrile/water with 0.1% formic acid) to give 22.0 mg (86% purity, 69% yield) of the target compound.

¹H-NMR (400 MHz, DMSO-d6) δ[ppm]: 1.220 (0.40), 1.231 (0.44), 1.380 (16.00), 1.383 (13.04), 1.387 (10.91), 2.331 (0.42), 2.518 (2.30), 2.523 (1.49), 2.673 (0.43), 8.042 (0.42), 8.084 (0.47), 8.089 (0.44), 8.415 (0.70), 8.762 (0.64), 8.767 (0.61).

### Example 22-A

### N⁶—{N-[(1r,4S)-4-(aminomethyl)cyclohexane-1-carbonyl]-3-(quinolin-3-yl)-L-alanyl}-N²-{[(1S)-1,3-dicarboxypropyl]carbamoyl}-L-lysine

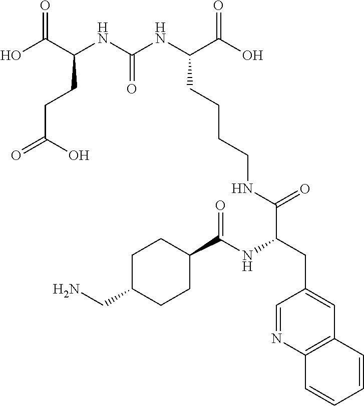 [SMILES: NC[C@H]1CC[C@H](C(=O)N[C@@H](Cc2cnc3ccccc3c2)C(=O)NCCCC[C@H](NC(=O)N[C@@H](CCC(=O)O)C(=O)O)C(=O)O)CC1]

tri-tert-butyl (3S,10S,14S)-1-[(1r,4S)-4-(aminomethyl)cyclohexyl]-1,4,12-trioxo-3-[(quinolin-3-yl)methyl]-2,5,11,13-tetraazahexadecane-10,14,16-tricarboxylate (10.0 mg, 12.1 μmol) was solubilised in DCM (390 μl), TFA (190 μl, 2.4 mmol) was added and the mixture was stirred under argon for 43 h at rt. The mixture was evaporated and purified by preparative HPLC (C18, acetonitrile/water with 0.1% formic acid) to give 6.00 mg (95% purity, 72% yield) of the target compound.

LC-MS (Method 1): Rt=0.52 min; MS (ESIpos): m/z=658 [M+H]⁺

¹H-NMR (400 MHz, DMSO-d6) δ[ppm]: 0.830 (0.53), 0.861 (0.83), 0.892 (0.65), 0.970 (0.47), 1.002 (0.41), 1.188 (0.65), 1.232 (1.83), 1.252 (1.18), 1.316 (0.89), 1.334 (1.00), 1.353 (0.89), 1.384 (1.42), 1.392 (1.42), 1.461 (0.89), 1.476 (0.77), 1.496 (0.77), 1.582 (0.41), 1.596 (0.47), 1.636 (1.12), 1.672 (0.94), 1.692 (0.89), 1.714 (0.83), 1.728 (0.83), 1.750 (0.41), 1.907 (0.53), 1.924 (0.47), 2.085 (0.59), 2.209 (0.65), 2.227 (1.00), 2.246 (1.12), 2.268 (0.59), 2.337 (1.18), 2.518 (16.00), 2.523 (10.57), 2.534 (3.37), 2.548 (5.49), 2.562 (2.66), 2.598 (0.89), 2.614 (1.36), 2.629 (0.89), 2.674 (2.54), 2.679 (1.18), 2.927 (0.47), 2.950 (0.65), 2.961 (0.89), 2.985 (0.83), 2.997 (0.71), 3.011 (0.59), 3.044 (0.47), 3.060 (0.59), 3.077 (0.47), 3.139 (0.65), 3.150 (0.71), 3.173 (0.59), 3.185 (0.53), 3.532 (4.37), 4.016 (0.71), 4.029 (0.71), 4.069 (0.41), 4.082 (0.53), 4.090 (0.77), 4.102 (0.77), 4.565 (0.41), 4.574 (0.59), 4.587 (0.59), 6.276 (1.12), 6.297 (1.06), 6.311 (1.18), 6.332 (1.06), 7.569 (1.00), 7.586 (2.18), 7.604 (1.77), 7.694 (0.71), 7.698 (0.77), 7.715 (1.18), 7.732 (0.59), 7.736 (0.59), 7.876 (1.12), 7.894 (1.00), 7.972 (1.42), 7.993 (1.18), 8.033 (1.30), 8.053 (1.83), 8.123 (1.48), 8.786 (2.01), 8.791 (1.83).

Intermediate 98 Monomer

### 2,2′-{[(3-{[2-({1-[2-({[(1S,4r)-4-{[(7S,11S,18S)-7,11-bis(tert-butoxycarbonyl)-2,2-dimethyl-4,9,17-trioxo-19-(quinolin-3-yl)-3-oxa-8,10,16-triazanonadecan-18-yl]carbamoyl}cyclohexyl]methyl}amino)-2-oxoethyl]-3-hydroxy-6-methyl-2-oxo-1,2-dihydropyridine-4-carbonyl}amino)ethyl](2-{[1-(carboxymethyl)-3-hydroxy-6-methyl-2-oxopyridine-4(2H)-carbonyl]amino}ethyl)amino}propyl)azanediyl]bis[(ethane-2,1-diyl)carbamoyl(3-hydroxy-6-methyl-2-oxopyridine-4,1(2H)-diyl)]}diacetic acid

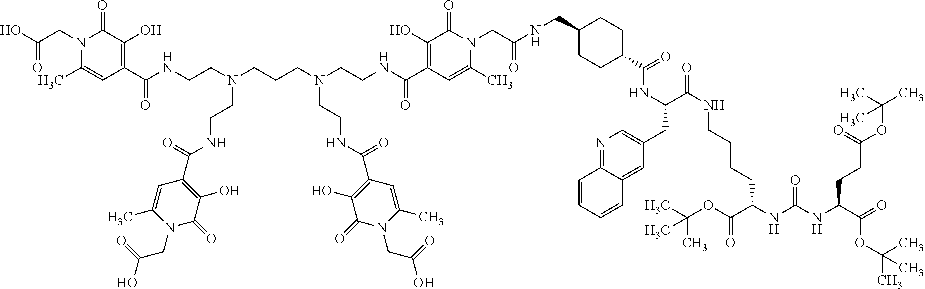 [SMILES: Cc1cc(C(=O)NCCN(CCCN(CCNC(=O)c2cc(C)n(CC(=O)O)c(=O)c2O)CCNC(=O)c2cc(C)n(CC(=O)NC[C@H]3CC[C@H](C(=O)N[C@@H](Cc4cnc5ccccc5c4)C(=O)NCCCC[C@H](NC(=O)N[C@@H](CCC(=O)OC(C)(C)C)C(=O)OC(C)(C)C)C(=O)OC(C)(C)C)CC3)c(=O)c2O)CCNC(=O)c2cc(C)n(CC(=O)O)c(=O)c2O)c(O)c(=O)n1CC(=O)O]

2,2′,2″,2′″-(propane-1,3-diylbis{nitrilobis[(ethane-2,1-diyl)carbamoyl(3-hydroxy-6-methyl-2-oxopyridine-4,1(2H)-diyl)]})tetraacetic acid (5.38 mg, 4.97 μmol) in 540 μL NMP, tri-tert-butyl (3S,10S,14S)-1-[(1 r,4S)-4-(aminomethyl)cyclohexyl]-1,4,12-trioxo-3-[(quinolin-3-yl)methyl]-2,5,11,13-tetraazahexadecane-10,14,16-tricarboxylate (5.00 mg, 6.06 μmol) in 500 μL NMP and PyAOP (4.74 mg, 9.09 μmol) in 474 μL NMP are mixed in a vial. DIPEA (8.7 μL, 50 μmol) is added. Reaction mix is quenched with water (2 mL), diluted with 38% ACN/water/0.1% TFA (6 mL) and the products purified by preparative HPLC (RP-HPLC using Äkta pure system, Column: Phenomenex Luna 5 μm C18(2) 100 Å, 250×21.2 mm Mobile phase: Water/0.1% TFA; ACN Gradient: 30-80% B over 40 min Flow: 10 mL/min Detection: UV 280/335 nm tR product: 27 min) affording 2.9 mg of the target compound.

LC-MS (Method K, gradient: 10-70% B over 3 min): Rₜ=1.77 min; MS (ESIpos): m/z=1891.0 [M+H]⁺

Intermediate 99 Dimer

### 2,2′-{propane-1,3-diylbis[{[2-({1-[2-({[(1S,4r)-4-{[(7S,11S,18S)-7,11-bis(tert-butoxycarbonyl)-2,2-dimethyl-4,9,17-trioxo-19-(quinolin-3-yl)-3-oxa-8,10,16-triazanonadecan-18-yl]carbamoyl}cyclohexyl]methyl}amino)-2-oxoethyl]-3-hydroxy-6-methyl-2-oxo-1,2-dihydropyridine-4-carbonyl}amino)ethyl]azanediyl}ethane-2,1-diylcarbamoyl(3-hydroxy-6-methyl-2-oxopyridine-4,1(2H)-diyl)]}diacetic acid

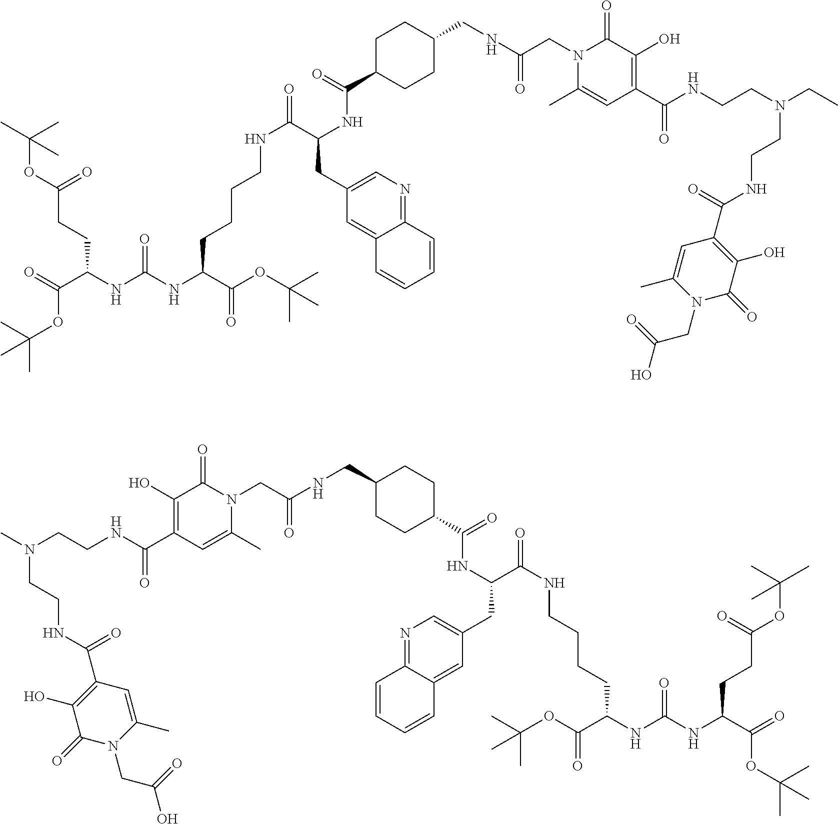

2,2′,2″,2′″-(propane-1,3-diylbis{nitrilobis[(ethane-2,1-diyl)carbamoyl(3-hydroxy-6-methyl-2-oxopyridine-4,1(2H)-diyl)]})tetraacetic acid (5.38 mg, 4.97 μmol) in 540 μL NMP, tri-tert-butyl (3S,10S,14S)-1-[(1r,4S)-4-(aminomethyl)cyclohexyl]-1,4,12-trioxo-3-[(quinolin-3-yl)methyl]-2,5,11,13-tetraazahexadecane-10,14,16-tricarboxylate (5.00 mg, 6.06 μmol) in 500 μL NMP and PyAOP (4.74 mg, 9.09 μmol) in 474 μL NMP are mixed in a vial. DIPEA (8.7 μL, 50 μmol) is added. Reaction mix is quenched with water (2 mL), diluted with 38% ACN/water/0.1% TFA (6 mL) and the products purified by preparative HPLC (RP-HPLC using Äkta pure system, Column: Phenomenex Luna 5 μm C18(2) 100 Å, 250×21.2 mm Mobile phase: Water/0.1% TFA; ACN Gradient: 30-80% B over 40 min Flow: 10 mL/min Detection: UV 280/335 nm tR product: 33 min) affording 2.7 mg of the target compound.

LC-MS (Method K, gradient: 10-70% B over 3 min): Rₜ=2.33 min; MS (ESIpos): m/z=1348.8 [M+2H]²⁺

Intermediate 100 Trimer

### {4-[(2-{(3-{bis[2-({1-[2-({[(1S,4r)-4-{[(7S,11S,18S)-7,11-bis(tert-butoxycarbonyl)-2,2-dimethyl-4,9,17-trioxo-19-(quinolin-3-yl)-3-oxa-8,10,16-triazanonadecan-18-yl]carbamoyl}cyclohexyl]methyl}amino)-2-oxoethyl]-3-hydroxy-6-methyl-2-oxo-1,2-dihydropyridine-4-carbonyl}amino)ethyl]amino}propyl)[2-({1-[2-({[(1S,4r)-4-{[(7S,11S,18S)-7,11-bis(tert-butoxycarbonyl)-2,2-dimethyl-4,9,17-trioxo-19-(quinolin-3-yl)-3-oxa-8,10,16-triazanonadecan-18-yl]carbamoyl}cyclohexyl]methyl}amino)-2-oxoethyl]-3-hydroxy-6-methyl-2-oxo-1,2-dihydropyridine-4-carbonyl}amino)ethyl]amino}ethyl)carbamoyl]-3-hydroxy-6-methyl-2-oxopyridin-1(2H)-yl}acetic acid

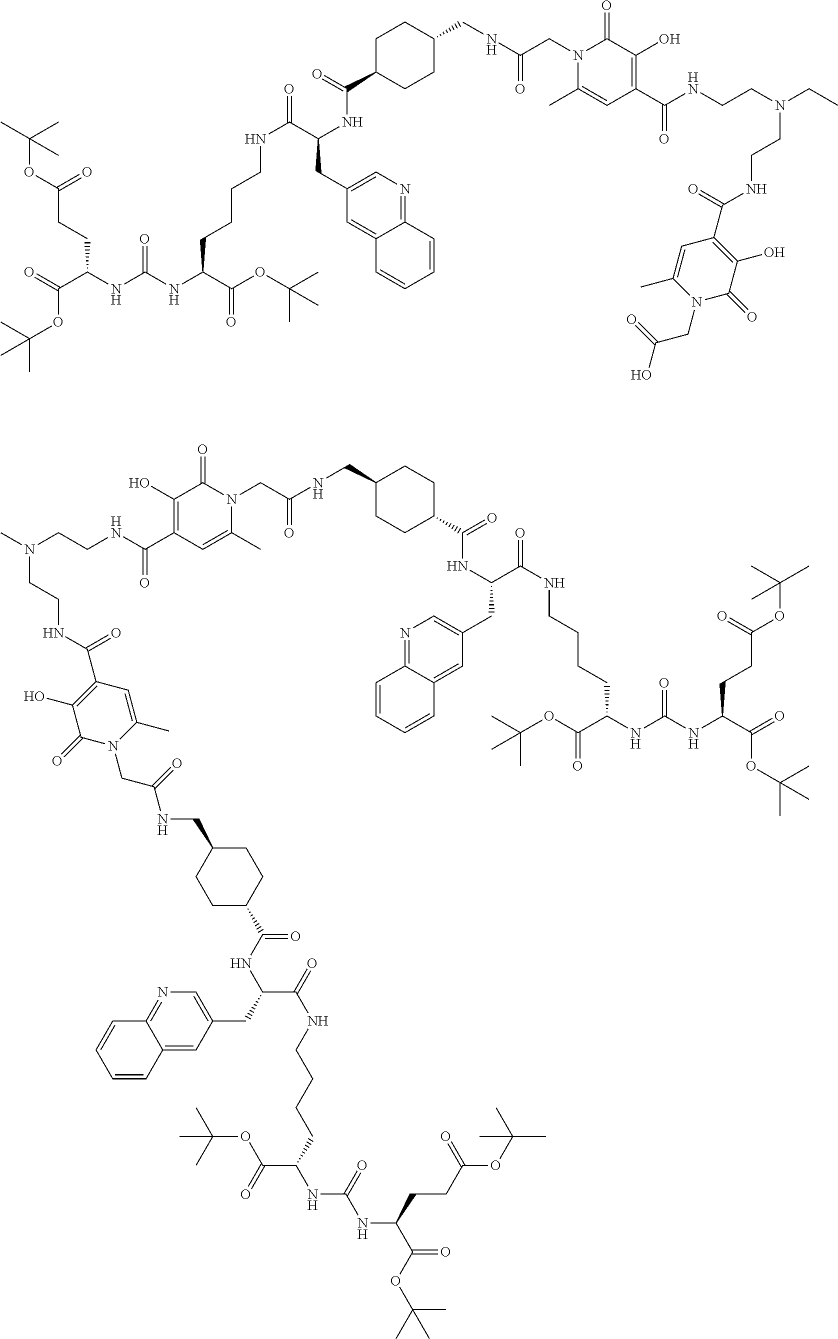

2,2′,2″,2′″-(propane-1,3-diylbis{nitrilobis[(ethane-2,1-diyl)carbamoyl(3-hydroxy-6-methyl-2-oxopyridine-4,1(2H)-diyl)]})tetraacetic acid (5.38 mg, 4.97 μmol) in 540 μL NMP, tri-tert-butyl (3S,10S,14S)-1-[(1 r,4S)-4-(aminomethyl)cyclohexyl]-1,4,12-trioxo-3-[(quinolin-3-yl)methyl]-2,5,11,13-tetraazahexadecane-10,14,16-tricarboxylate (5.00 mg, 6.06 μmol) in 500 μL NMP and PyAOP (4.74 mg, 9.09 μmol) in 474 μL NMP are mixed in a vial. DIPEA (8.7 μL, 50 μmol) is added. Reaction mix is quenched with water (2 mL), diluted with 38% ACN/water/0.1% TFA (6 mL) and the products purified by preparative HPLC (RP-HPLC using Äkta pure system, Column: Phenomenex Luna 5 μm C18(2) 100 Å, 250×21.2 mm Mobile phase: Water/0.1% TFA; ACN Gradient: 30-80% B over 40 min Flow: 10 mL/min Detection: UV 280/335 nm tR product: 37 min) affording 1.4 mg of the target compound.

LC-MS (Method K, gradient: 10-70% B over 3 min): Rₜ=2.66 min; MS (ESIpos): m/z=1752.8 [M+2H]²⁺

### Example 22-C Monomer

### N⁶—{N-[(1r,4S)-4-({2-[4-({2-[{3-[bis(2-{[1-(carboxymethyl)-3-hydroxy-6-methyl-2-oxo-1,2-dihydropyridine-4-carbonyl]amino}ethyl)amino]propyl}(2-{[1-(carboxymethyl)-3-hydroxy-6-methyl-2-oxo-1,2-dihydropyridine-4-carbonyl]amino}ethyl)amino]ethyl}carbamoyl)-3-hydroxy-6-methyl-2-oxopyridin-1(2H)-yl]acetamido}methyl)cyclohexane-1-carbonyl]-3-(quinolin-3-yl)-L-alanyl}-N²-{[(1S)-1,3-dicarboxypropyl]carbamoyl}-L-lysine

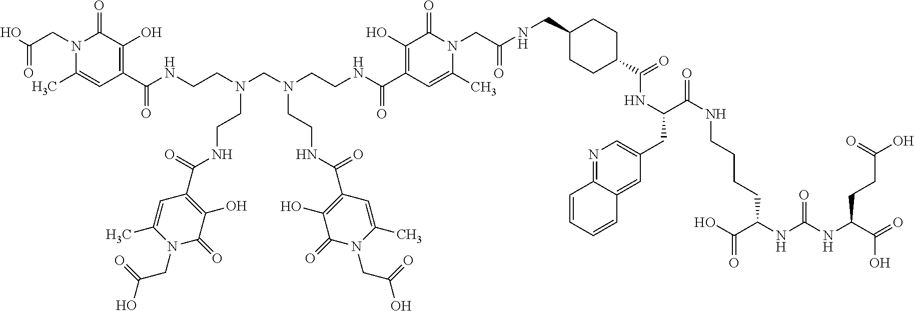 [SMILES: Cc1cc(C(=O)NCCN(CCNC(=O)c2cc(C)n(CC(=O)O)c(=O)c2O)CN(CCNC(=O)c2cc(C)n(CC(=O)O)c(=O)c2O)CCNC(=O)c2cc(C)n(CC(=O)NC[C@H]3CC[C@H](C(=O)N[C@@H](Cc4cnc5ccccc5c4)C(=O)NCCCC[C@H](NC(=O)N[C@@H](CCC(=O)O)C(=O)O)C(=O)O)CC3)c(=O)c2O)c(O)c(=O)n1CC(=O)O]

2,2′-{[(3-{[2-({1-[2-({[(1S,4r)-4-{[(7S,11S,18S)-7,11-bis(tert-butoxycarbonyl)-2,2-dimethyl-4,9,17-trioxo-19-(quinolin-3-yl)-3-oxa-8,10,16-triazanonadecan-18-yl]carbamoyl}cyclohexyl]methyl}amino)-2-oxoethyl]-3-hydroxy-6-methyl-2-oxo-1,2-dihydropyridine-4-carbonyl}amino)ethyl](2-{[1-(carboxymethyl)-3-hydroxy-6-methyl-2-oxopyridine-4(2H)-carbonyl]amino}ethyl)amino}propyl)azanediyl]bis[(ethane-2,1-diyl)carbamoyl(3-hydroxy-6-methyl-2-oxopyridine-4,1(2H)-diyl)]}diacetic acid (2.90 mg, 1.53 μmol) is treated with 80% TFA in water (0.5 mL). Water (18 mL) is and the reaction mixture is lyophilised affording 2.90 mg (95% purity, 104% yield) of the target compound

LC-MS (Method K, gradient: 10-70% B over 3 min): Rₜ=0.83 min; MS (ESIpos): m/z=1221.8 [M+H]⁺

### Example 22-C Monomer-²²⁷Th

### [N⁶—{N-[(1r,4S)-4-({2-[4-({2-[{3-[bis(2-{[1-(carboxymethyl)-3-(hydroxy-kappaO)-6-methyl-2-(oxo-kappaO)-1,2-dihydropyridine-4-carbonyl]amino}ethyl)amino]propyl}(2-{[1-(carboxymethyl)-3-(hydroxy-kappaO)-6-methyl-2-(oxo-kappaO)-1,2-dihydropyridine-4-carbonyl]amino}ethyl)amino]ethyl}carbamoyl)-3-(hydroxy-kappaO)-6-methyl-2-(oxo-kappaO)pyridin-1(2H)-yl]acetamido}methyl)cyclohexane-1-carbonyl]-3-(quinolin-3-yl)-L-alanyl}-N²-{[(1S)-1,3-dicarboxypropyl]carbamoyl}-L-lysinato(4−)](²²⁷Th)Thorium

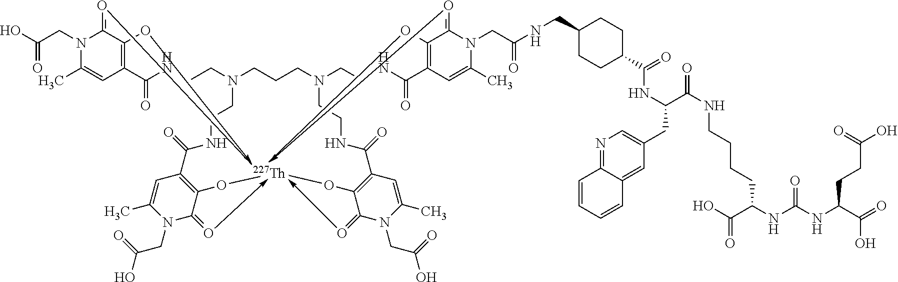

N⁶—{N-[(1r,4S)-4-({2-[4-({2-[{3-[bis(2-{[1-(carboxymethyl)-3-hydroxy-6-methyl-2-oxo-1,2-dihydropyridine-4-carbonyl]amino}ethyl)amino]propyl}(2-{[1-(carboxymethyl)-3-hydroxy-6-methyl-2-oxo-1,2-dihydropyridine-4-carbonyl]amino}ethyl)amino]ethyl}carbamoyl)-3-hydroxy-6-methyl-2-oxopyridin-1(2H)-yl]acetamido}methyl)cyclohexane-1-carbonyl]-3-(quinolin-3-yl)-L-alanyl}-N²-{[(1S)-1,3-dicarboxypropyl]carbamoyl}-L-lysine (4 μg) dissolved in 147 μL 30 mM citrate buffer (pH 5.5) containing 0.5 mg/mL pABA was mixed with thorium-227 in 0.5 M HCl (2 μL) at 0.3 MBq/nmol specific activity and RAC of 4.6 MBq/mL. 1M carbonate buffer pH 9 (15 μL) was added and mixture incubated for 60 min. The labelling efficiency was determined to be 97% by iTLC.

### Example 22-C Dimer

### (3S,10S,14S,3'S,10'S,14'S)-1,1′-(propane-1,3-diylbis{[(2-{[1-(carboxymethyl)-3-hydroxy-6-methyl-2-oxo-1,2-dihydropyridine-4-carbonyl]amino}ethyl)azanediyl]ethane-2,1-diylcarbamoyl(3-hydroxy-6-methyl-2-oxopyridine-4,1(2H)-diyl)(1-oxoethane-2,1-diyl)azanediylmethylene(1r,4S)cyclohexane-4,1-diyl})bis{1,4,12-trioxo-3-[(quinolin-3-yl)methyl]-2,5,11,13-tetraazahexadecane-10,14,16-tricarboxylic acid}

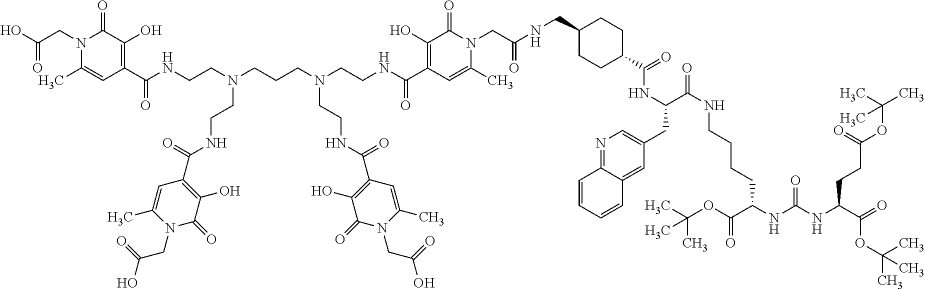 [SMILES: Cc1cc(C(=O)NCCN(CCCN(CCNC(=O)c2cc(C)n(CC(=O)O)c(=O)c2O)CCNC(=O)c2cc(C)n(CC(=O)NC[C@H]3CC[C@H](C(=O)N[C@@H](Cc4cnc5ccccc5c4)C(=O)NCCCC[C@H](NC(=O)N[C@@H](CCC(=O)OC(C)(C)C)C(=O)OC(C)(C)C)C(=O)OC(C)(C)C)CC3)c(=O)c2O)CCNC(=O)c2cc(C)n(CC(=O)O)c(=O)c2O)c(O)c(=O)n1CC(=O)O]

2,2′-{propane-1,3-diylbis[{[2-({1-[2-({[(1S,4r)-4-{[(7S,11S,18S)-7,11-bis(tert-butoxycarbonyl)-2,2-dimethyl-4,9,17-trioxo-19-(quinolin-3-yl)-3-oxa-8,10,16-triazanonadecan-18-yl]carbamoyl}cyclohexyl]methyl}amino)-2-oxoethyl]-3-hydroxy-6-methyl-2-oxo-1,2-dihydropyridine-4-carbonyl}amino)ethyl]azanediyl}ethane-2,1-diylcarbamoyl(3-hydroxy-6-methyl-2-oxopyridine-4,1(2H)-diyl)]}diacetic acid (2.70 mg, 1.00 μmol) is treated with 80% TFA in water (0.5 mL). Water (18 mL) is and the reaction mixture is lyophilised affording 2.8 mg of the target compound

LC-MS (Method K, gradient: 10-70% B over 3 min): Rₜ=0.93 min; MS (ESIpos): m/z=1180.6 [M+2H]²⁺

### Example 22-C Dimer-²²⁷Th

### (3S,10S,14S,3'S,10'S,14'S)-1,1′-(propane-1,3-diylbis{[(2-{[1-(carboxymethyl)-3-(hydroxy-kappaO)-6-methyl-2-(oxo-kappaO)-1,2-dihydropyridine-4-carbonyl]amino}ethyl)azanediyl]ethane-2,1-diylcarbamoyl[3-(hydroxy-kappaO)-6-methyl-2-(oxo-kappaO)pyridine-4,1(2H)-diyl](1-oxoethane-2,1-diyl)azanediylmethylene(1r,4S)cyclohexane-4,1-diyl})bis{1,4,12-trioxo-3-[(quinolin-3-yl)methyl]-2,5,11,13-tetraazahexadecane-10,14,16-tricarboxylato}(4−)(²²⁷Th)Thorium

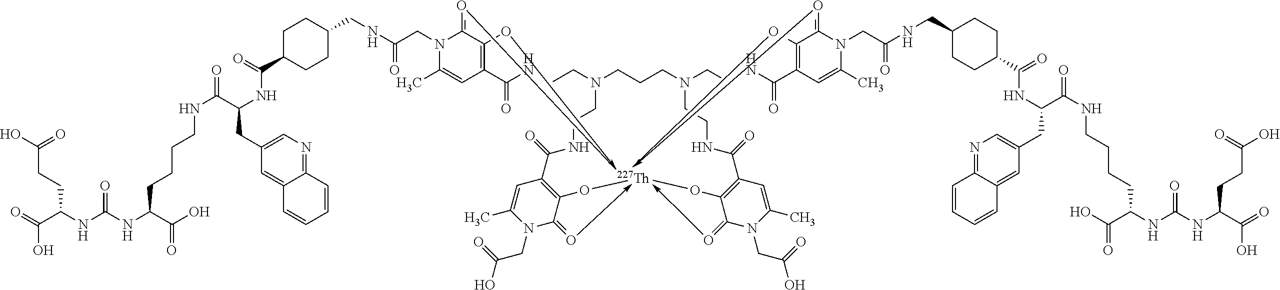

(3S,10S,14S,3'S,10'S,14'S)-1,1′-(propane-1,3-diylbis{[(2-{[1-(carboxymethyl)-3-hydroxy-6-methyl-2-oxo-1,2-dihydropyridine-4-carbonyl]amino}ethyl)azanediyl]ethane-2,1-diylcarbamoyl(3-hydroxy-6-methyl-2-oxopyridine-4,1(2H)-diyl)(1-oxoethane-2,1-diyl)azanediylmethylene(1r,4S)cyclohexane-4,1-diyl})bis{1,4,12-trioxo-3-[(quinolin-3-yl)methyl]-2,5,11,13-tetraazahexadecane-10,14,16-tricarboxylic acid} (8 μg) dissolved in 193 μL 30 mM citrate buffer (pH 5.5) containing 0.5 mg/mL pABA was mixed with thorium-227 in 0.5 M HCl (2 μL) at 0.3 MBq/nmol specific activity and RAC of 4.5 MBq/mL. 1M carbonate buffer pH 9 (19 μL) was added and mixture incubated for 60 min. The labelling efficiency was determined to be 87% by iTLC.

### Example 22-C Trimer

### (3S,10S,14S,3'S,10'S,14'S)-1,1′-{[(3-{[2-({1-[2-({[(1S,4r)-4-{[(2S)-1-{[(5S)-5-carboxy-5-({[(1S)-1,3-dicarboxypropyl]carbamoyl}amino)pentyl]amino}-1-oxo-3-(quinolin-3-yl)propan-2-yl]carbamoyl}cyclohexyl]methyl}amino)-2-oxoethyl]-3-hydroxy-6-methyl-2-oxopyridine-4(2H)-carbonyl}amino)ethyl](2-{[1-(carboxymethyl)-3-hydroxy-6-methyl-2-oxo-1,2-dihydropyridine-4-carbonyl]amino}ethyl)amino}propyl)azanediyl]bis[(ethane-2,1-diyl)carbamoyl(3-hydroxy-6-methyl-2-oxopyridine-4,1(2H)-diyl)(1-oxoethane-2,1-diyl)azanediylmethylene(1r,4S)cyclohexane-4,1-diyl]}bis{1,4,12-trioxo-3-[(quinolin-3-yl)methyl]-2,5,11,13-tetraazahexadecane-10,14,16-tricarboxylic acid}

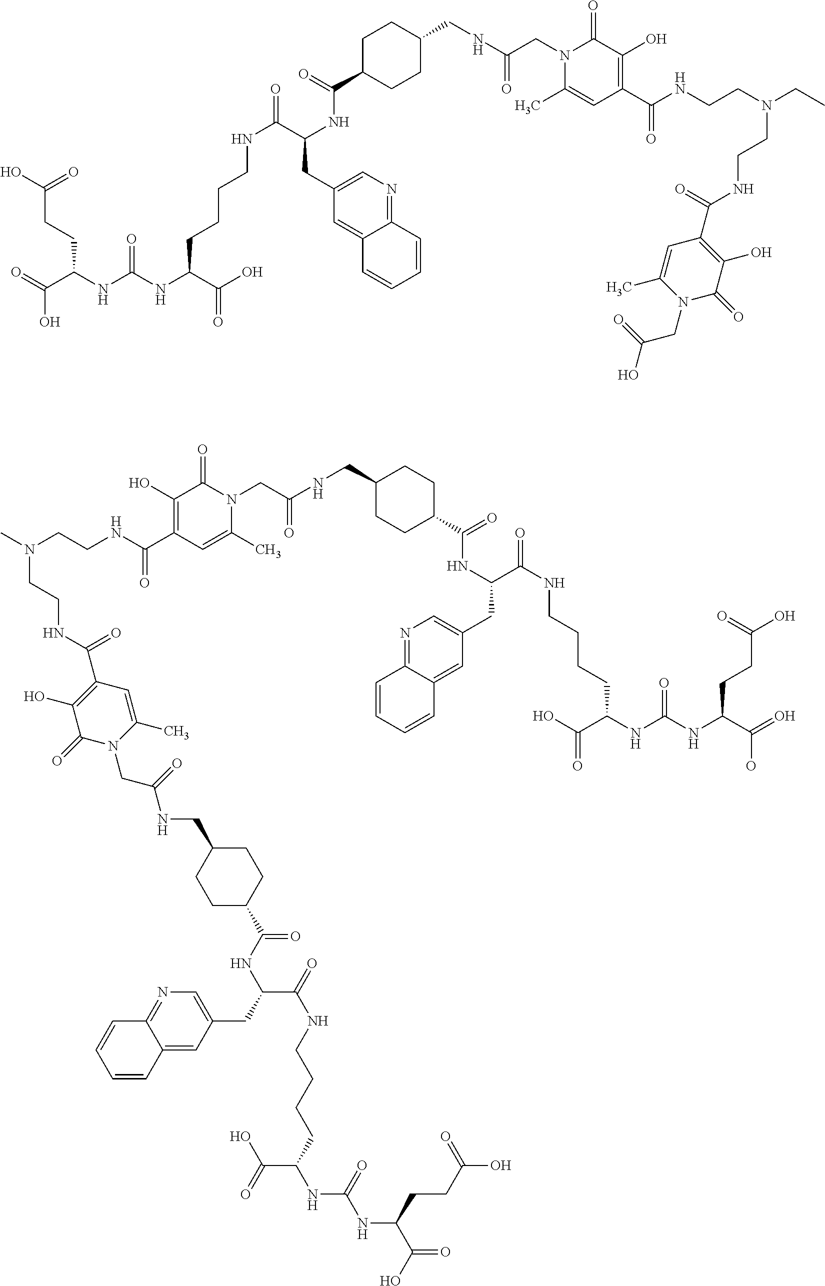

{4-[(2-{(3-{bis[2-({1-[2-({[(1S,4r)-4-{[(7S,11S,18S)-7,11-bis(tert-butoxycarbonyl)-2,2-dimethyl-4,9,17-trioxo-19-(quinolin-3-yl)-3-oxa-8,10,16-triazanonadecan-18-yl]carbamoyl}cyclohexyl]methyl}amino)-2-oxoethyl]-3-hydroxy-6-methyl-2-oxo-1,2-dihydropyridine-4-carbonyl}amino)ethyl]amino}propyl)[2-({1-[2-({[(1S,4r)-4-{[(7S,11S,18S)-7,11-bis(tert-butoxycarbonyl)-2,2-dimethyl-4,9,17-trioxo-19-(quinolin-3-yl)-3-oxa-8,10,16-triazanonadecan-18-yl]carbamoyl}cyclohexyl]methyl}amino)-2-oxoethyl]-3-hydroxy-6-methyl-2-oxo-1,2-dihydropyridine-4-carbonyl}amino)ethyl]amino}ethyl)carbamoyl]-3-hydroxy-6-methyl-2-oxopyridin-1(2H)-yl}acetic acid (1.40 mg, 0.400 μmol) is treated with 80% TFA in water (0.5 mL) 01:00. Water (18 mL) is and the reaction mixture is lyophilised affording 2.8 mg of the target compound.

LC-MS (Method K, gradient: 10-70% B over 3 min): Rₜ=1.02 min; MS (ESIpos): m/z=1500.0 [M+2H]²⁺

### Example 22-C Trimer-²²⁷Th

### (3S,10S,14S,3'S,10'S,14'S)-1,1′-{[(3-{[2-({1-[2-({[(1S,4r)-4-{[(2S)-1-{[(5S)-5-carboxy-5-({[(1S)-1,3-dicarboxypropyl]carbamoyl}amino)pentyl]amino}-1-oxo-3-(quinolin-3-yl)propan-2-yl]carbamoyl}cyclohexyl]methyl}amino)-2-oxoethyl]-3-(hydroxy-kappaO)-6-methyl-2-(oxo-kappaO)pyridine-4(2H)-carbonyl}amino)ethyl](2-{[1-(carboxymethyl)-3-(hydroxy-kappaO)-6-methyl-2-(oxo-kappaO)-1,2-dihydropyridine-4-carbonyl]amino}ethyl)amino}propyl)azanediyl]bis[(ethane-2,1-diyl)carbamoyl[3-(hydroxy-kappaO)-6-methyl-2-(oxo-kappaO)pyridine-4,1(2H)-diyl](1-oxoethane-2,1-diyl)azanediylmethylene(1r,4S)cyclohexane-4,1-diyl]}bis{1,4,12-trioxo-3-[(quinolin-3-yl)methyl]-2,5,11,13-tetraazahexadecane-10,14,16-tricarboxylato}(4−)(²²⁷Th)Thoriuum

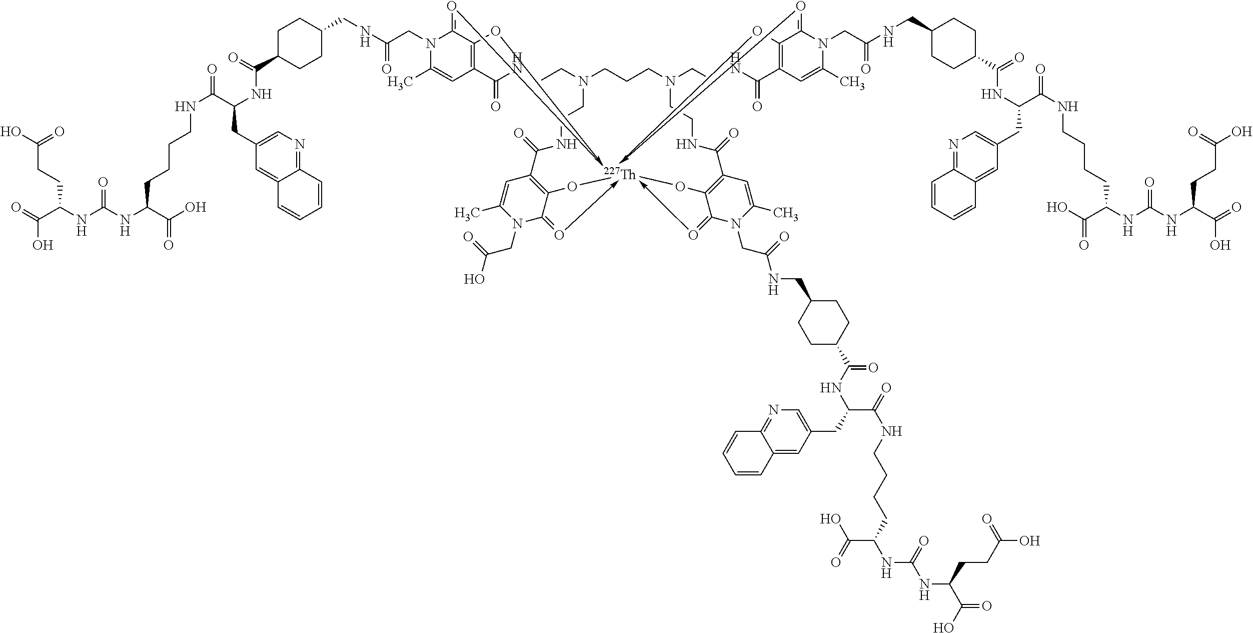

(3S,10S,14S,3'S,10'S,14'S)-1,1′-{[(3-{[2-({1-[2-({[(1S,4r)-4-{[(2S)-1-{[(5S)-5- carboxy-5-({[(1S)-1,3-dicarboxypropyl]carbamoyl}amino)pentyl]amino}-1-oxo-3-(quinolin-3-yl)propan-2-yl]carbamoyl}cyclohexyl]methyl}amino)-2-oxoethyl]-3-hydroxy-6-methyl-2-oxopyridine-4(2H)-carbonyl}amino)ethyl](2-{[1-(carboxymethyl)-3-hydroxy-6-methyl-2-oxo-1,2-dihydropyridine-4-carbonyl]amino}ethyl)amino}propyl)azanediyl]bis[(ethane-2,1-diyl)carbamoyl(3-hydroxy-6-methyl-2-oxopyridine-4,1(2H)-diyl)(1-oxoethane-2,1-diyl)azanediylmethylene(1r,4S)cyclohexane-4,1-diyl]}bis{1,4,12-trioxo-3-[(quinolin-3-yl)methyl]-2,5,11,13-tetraazahexadecane-10,14,16-tricarboxylic acid} (9 μg) dissolved in 172 μL 30 mM citrate buffer (pH 5.5) containing 0.5 mg/mL pABA was mixed with thorium-227 in 0.5 M HCl (2 μL) at 0.3 MBq/nmol specific activity and RAC of 4.5 MBq/mL. 1M carbonate buffer pH 9 (17 μL) was added and mixture incubated for 4 hrs. The labelling efficiency was determined to be 90% by iTLC.

#23 Linker

Intermediate 101

### tri-tert-butyl (4S,11S,15S)-1-[4-({[(9H-fluoren-9-yl)methoxy]carbonyl}amino)phenyl]-4-[(naphthalen-2-yl)methyl]-2,5,13-trioxo-3,6,12,14-tetraazaheptadecane-11,15,17-tricarboxylate

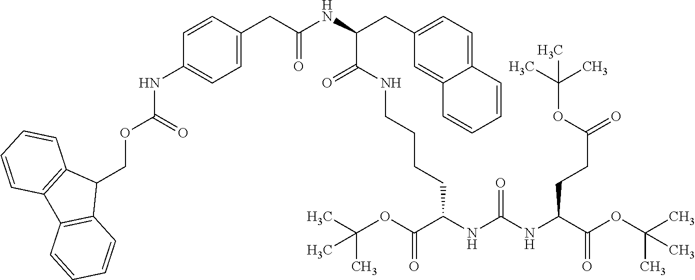 [SMILES: CC(C)(C)OC(=O)CC[C@H](NC(=O)N[C@@H](CCCCNC(=O)[C@H](Cc1ccc2ccccc2c1)NC(=O)Cc1ccc(NC(=O)OCC2c3ccccc3-c3ccccc32)cc1)C(=O)OC(C)(C)C)C(=O)OC(C)(C)C]

di-tert-butyl (2S)-2-({[(2S)-6-{[(2S)-2-amino-3-(naphthalen-2-yl)propanoyl]amino}-1-tert-butoxy-1-oxohexan-2-yl]carbamoyl}amino)pentanedioate (319 mg, 90% purity, 419 μmol) and [4-({[(9H-fluoren-9-yl)methoxy]carbonyl}amino)phenyl]acetic acid (130 mg, 349 μmol) were solubilised in DMF (2.7 ml), 4-methylmorpholine (260 μl, 1.0 mmol, CAS-RN: 109-02-4) and COMU (150 mg, 349 μmol) were added and the mixture was stirred under argon for 2 h at rt. The mixture was diluted with brine and extracted 3 times with DCM. The combined organic phases were dried and evaporated and purified by flash chromatography (SiO2, DCM/Ethanol gradient 0%-10%) to give 219 mg (80% purity, 40% yield) of the target compound.

LC-MS (Method 1): Rₜ=1.64 min; MS (ESIpos): m/z=1041 [M+H]⁺

¹H-NMR (400 MHz, DMSO-d6) δ[ppm]: 1.250 (0.58), 1.268 (1.11), 1.286 (0.64), 1.377 (12.53), 1.381 (16.00), 2.518 (3.47), 2.522 (2.21), 2.673 (0.62), 2.737 (9.57), 3.053 (1.07), 3.065 (1.32), 3.077 (1.21), 3.301 (0.42), 3.547 (1.08), 3.559 (1.10), 3.570 (1.01), 7.348 (0.59), 7.366 (0.40), 7.408 (0.40), 7.428 (0.74), 7.435 (0.51), 7.639 (0.42), 7.744 (0.81), 7.760 (0.59), 7.765 (0.64), 7.902 (0.65), 7.921 (0.56).

Intermediate 102

### tri-tert-butyl (4S,11S,15S)-1-(4-aminophenyl)-4-[(naphthalen-2-yl)methyl]-2,5,13-trioxo-3,6,12,14-tetraazaheptadecane-11,15,17-tricarboxylate

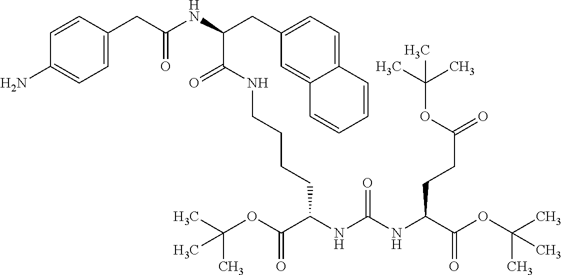 [SMILES: CC(C)(C)OC(=O)CC[C@H](NC(=O)N[C@@H](CCCCNC(=O)[C@H](Cc1ccc2ccccc2c1)NC(=O)Cc1ccc(N)cc1)C(=O)OC(C)(C)C)C(=O)OC(C)(C)C]

tri-tert-butyl (4S,11S,15S)-1-[4-({[(9H-fluoren-9-yl)methoxy]carbonyl}amino)phenyl]-4-[(naphthalen-2-yl)methyl]-2,5,13-trioxo-3,6,12,14-tetraazaheptadecane-11,15,17-tricarboxylate (219 mg, 85% purity, 179 μmol) was solubilised in DMF (1.4 ml), piperidine (350 μl, 3.6 mmol) was added and the mixture was stirred under argon overnight at rt. The mixture was evaporated and purified by preparative HPLC (C18, acetonitrile/water with 0.1% formic acid) to give 126 mg (90% purity, 77% yield) of the target compound.

LC-MS (Method 1): Rₜ=1.40 min; MS (ESIpos): m/z=819 [M+H]⁺

¹H-NMR (400 MHz, DMSO-d6) δ[ppm]: 1.382 (13.17), 1.386 (16.00), 2.518 (1.35), 2.522 (0.83), 3.166 (0.42), 3.200 (0.41), 4.833 (0.55), 6.281 (0.53), 6.322 (0.74), 6.343 (0.78), 6.703 (0.67), 6.724 (0.60), 7.464 (0.44), 7.648 (0.42).

### Example 23-A

### (4S,11S,15S)-1-(4-aminophenyl)-4-[(naphthalen-2-yl)methyl]-2,5,13-trioxo-3,6,12,14-tetraazaheptadecane-11,15,17-tricarboxylic acid

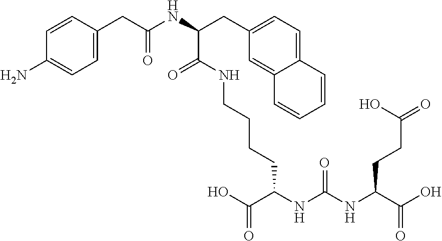 [SMILES: Nc1ccc(CC(=O)N[C@@H](Cc2ccc3ccccc3c2)C(=O)NCCCC[C@H](NC(=O)N[C@@H](CCC(=O)O)C(=O)O)C(=O)O)cc1]

tri-tert-butyl (4S,11S,15S)-1-(4-aminophenyl)-4-[(naphthalen-2-yl)methyl]-2,5,13-trioxo-3,6,12,14-tetraazaheptadecane-11,15,17-tricarboxylate (17.0 mg, 90% purity, 18.7 μmol) was solubilised in DCM (1 ml), TFA (29 μl, 370 μmol) was added and the mixture was stirred under argon overnight at rt. The mixture was evaporated and purified by preparative HPLC (C18, acetonitrile/water with 0.1% formic acid) to give 5.30 mg (95% purity, 41% yield) of the target compound.

LC-MS (Method 1): Rₜ=0.75 min; MS (ESIpos): m/z=650 [M+H]⁺

¹H-NMR (400 MHz, DMSO-d6) δ[ppm]: 1.206 (3.53), 1.212 (3.38), 1.225 (3.83), 1.262 (1.93), 1.279 (3.66), 1.297 (3.83), 1.316 (2.35), 1.404 (0.53), 1.423 (1.05), 1.440 (1.53), 1.456 (1.98), 1.475 (1.63), 1.494 (0.58), 1.543 (0.75), 1.561 (1.50), 1.575 (1.93), 1.594 (1.65), 1.608 (1.05), 1.628 (0.55), 1.669 (0.53), 1.689 (1.33), 1.707 (1.63), 1.724 (2.13), 1.741 (1.68), 1.752 (0.75), 1.761 (0.80), 1.839 (0.63), 1.856 (1.45), 1.872 (1.93), 1.891 (1.95), 1.905 (1.10), 1.925 (0.58), 2.190 (0.53), 2.214 (3.18), 2.221 (3.18), 2.235 (4.61), 2.240 (4.68), 2.252 (2.65), 2.261 (2.60), 2.283 (0.50), 2.332 (1.05), 2.518 (6.49), 2.523 (4.01), 2.539 (0.48), 2.673 (1.05), 2.727 (6.54), 2.736 (1.55), 2.887 (8.14), 2.902 (2.45), 2.924 (3.03), 2.935 (4.78), 2.957 (4.13), 2.966 (2.38), 2.985 (1.13), 2.996 (1.25), 3.013 (2.28), 3.028 (2.70), 3.045 (2.25), 3.062 (4.03), 3.075 (3.73), 3.095 (2.70), 3.109 (2.50), 3.135 (3.81), 3.171 (9.19), 3.200 (9.29), 3.235 (5.33), 3.546 (1.08), 3.982 (1.45), 3.995 (1.93), 4.002 (2.93), 4.015 (3.00), 4.022 (1.83), 4.035 (1.45), 4.057 (1.48), 4.078 (3.03), 4.091 (3.08), 4.112 (1.38), 4.492 (1.40), 4.506 (1.78), 4.514 (2.80), 4.527 (2.88), 4.535 (1.73), 4.548 (1.43), 6.282 (4.86), 6.302 (6.44), 6.323 (16.00), 6.328 (5.33), 6.340 (5.11), 6.344 (15.10), 6.351 (1.93), 6.703 (13.32), 6.724 (11.77), 6.972 (0.48), 6.993 (0.50), 7.316 (0.65), 7.339 (4.76), 7.343 (4.51), 7.360 (4.76), 7.364 (4.88), 7.430 (1.53), 7.433 (1.88), 7.447 (4.76), 7.450 (4.33), 7.460 (5.03), 7.466 (7.71), 7.470 (5.33), 7.480 (3.91), 7.484 (4.46), 7.497 (1.80), 7.501 (1.35), 7.655 (8.41), 7.763 (7.16), 7.773 (5.26), 7.784 (6.76), 7.791 (4.33), 7.795 (3.51), 7.838 (4.08), 7.842 (4.38), 7.860 (3.96), 7.950 (1.13), 7.977 (2.18), 7.991 (4.28), 8.005 (2.08), 8.060 (4.96), 8.081 (4.63), 8.147 (1.60), 8.213 (0.43), 8.217 (0.43).

Intermediate 103

tri-tert-butyl (4S,11S,15S)-4-[(naphthalen-2-yl)methyl]-2,5,13-trioxo-1-(4-{2-[4,7,10-tris(2-tert-butoxy-2-oxoethyl)-1,4,7,10-tetraazacyclododecan-1-yl]acetamido}phenyl)-3,6,12,14-tetraazaheptadecane-11,15,17-tricarboxylate

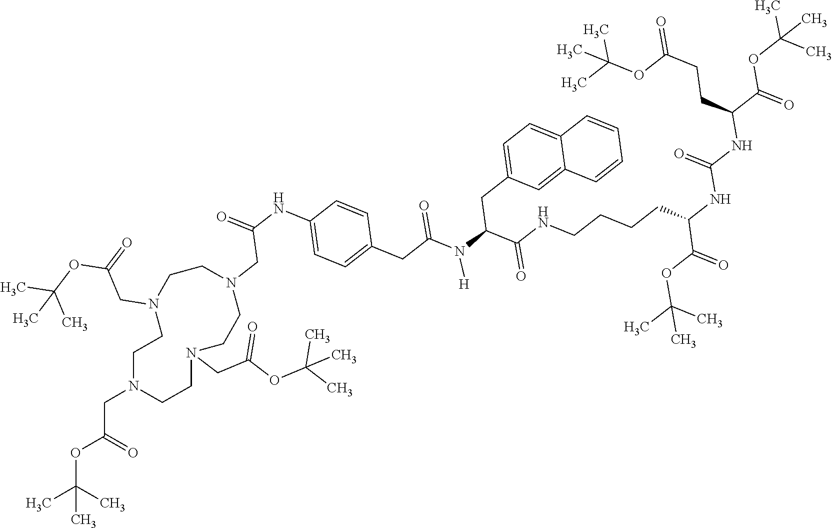 [SMILES: CC(C)(C)OC(=O)CC[C@H](NC(=O)N[C@@H](CCCCNC(=O)[C@H](Cc1ccc2ccccc2c1)NC(=O)Cc1ccc(NC(=O)CN2CCN(CC(=O)OC(C)(C)C)CCN(CC(=O)OC(C)(C)C)CCN(CC(=O)OC(C)(C)C)CC2)cc1)C(=O)OC(C)(C)C)C(=O)OC(C)(C)C]

N-[9,12-bis(2-tert-butoxy-2-oxoethyl)-2,2,6-trimethyl-4-oxo-3-oxa-6,9,12-triazatetradecan-14-yl]-N-ethylglycine (132 mg, 225 μmol), [(1H-benzotriazol-1-yl)oxy](dimethylamino)-N,N-dimethylmethaniminium hexafluoridophosphate(1−) (84.2 mg, 222 μmol; CAS-RN:94790-37-1) and N,N-diisopropylethylamine (36 μl, 220 μmol) were stirred in DMF (2 ml) for 10 min at rt. tri-tert-butyl (4S,11S,15S)-1-(4-aminophenyl)-4-[(naphthalen-2-yl)methyl]-2,5,13-trioxo-3,6,12,14-tetraazaheptadecane-11,15,17-tricarboxylate (57.5 mg, 80% purity, 56.2 μmol) was added and the mixture was stirred overnight at rt. The mixture was evaporated and purified by preparative HPLC (C18, acetonitrile/water with 0.1% formic acid) to give 45.0 mg (70% purity, 41% yield) of the target compound.

LC-MS (Method 1): Rₜ=1.26 min; MS (ESIpos): m/z=1373 [M+H]⁺

¹H-NMR (400 MHz, DMSO-d6) δ[ppm]: 1.232 (0.47), 1.342 (6.22), 1.380 (16.00), 1.384 (15.09), 1.408 (3.07), 1.429 (2.35), 1.467 (3.51), 2.084 (1.13), 2.331 (0.45), 2.518 (2.53), 2.522 (1.58), 2.539 (0.43), 2.673 (0.46), 2.888 (0.67), 2.896 (0.46), 3.065 (0.43), 7.367 (0.51), 7.462 (0.40), 7.754 (0.42), 7.775 (0.51).

### Example 23-B

### (4S,11S,15S)-4-[(naphthalen-2-yl)methyl]-2,5,13-trioxo-1-(4-{2-[4,7,10-tris(carboxymethyl)-1,4,7,10-tetraazacyclododecan-1-yl]acetamido}phenyl)-3,6,12,14-tetraazaheptadecane-11,15,17-tricarboxylic acid

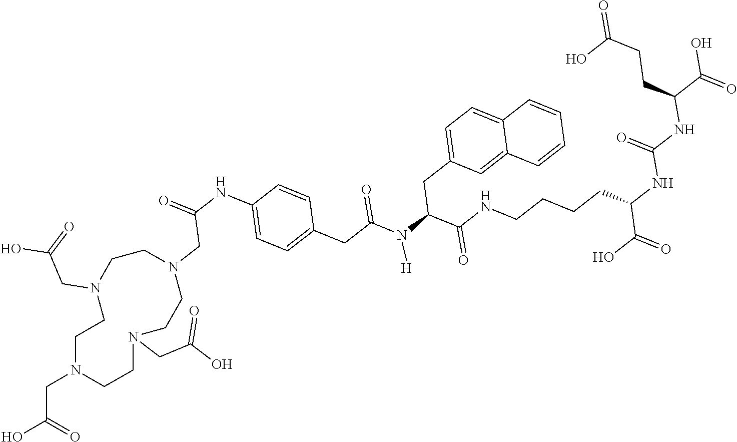 [SMILES: O=C(O)CC[C@H](NC(=O)N[C@@H](CCCCNC(=O)[C@H](Cc1ccc2ccccc2c1)NC(=O)Cc1ccc(NC(=O)CN2CCN(CC(=O)O)CCN(CC(=O)O)CCN(CC(=O)O)CC2)cc1)C(=O)O)C(=O)O]

tri-tert-butyl (4S,11S,15S)-4-[(naphthalen-2-yl)methyl]-2,5,13-trioxo-1-(4-{2-[4,7,10-tris(2-tert-butoxy-2-oxoethyl)-1,4,7,10-tetraazacyclododecan-1-yl]acetamido}phenyl)-3,6,12,14-tetraazaheptadecane-11,15,17-tricarboxylate (40.0 mg, 29.1 μmol) was solubilised in DCM (560 μl), TFA (166 mg, 1.46 mmol) was added and the mixture was stirred under argon overnight at rt. The mixture was evaporated and purified by preparative HPLC (C18, acetonitrile/water with 0.1% formic acid) to give 3.80 mg (90% purity, 11% yield) of the target compound.

LC-MS (Method 1): Rₜ=0.70 min; MS (ESIpos): m/z=1037 [M+H]⁺

¹H-NMR (400 MHz, DMSO-d6) δ[ppm]: 0.833 (0.45), 0.852 (0.83), 1.232 (5.86), 1.247 (2.29), 1.256 (2.29), 1.295 (1.49), 1.333 (0.83), 1.348 (1.60), 1.445 (0.61), 1.563 (0.59), 1.692 (0.51), 1.712 (0.64), 1.726 (0.51), 1.897 (0.59), 1.914 (0.59), 2.178 (0.61), 2.210 (0.91), 2.230 (1.36), 2.247 (1.46), 2.268 (0.88), 2.518 (5.35), 2.523 (3.86), 2.540 (7.48), 2.674 (1.41), 2.727 (1.97), 2.806 (1.01), 2.888 (2.42), 2.993 (2.98), 3.094 (1.54), 3.114 (1.44), 3.129 (1.46), 3.356 (16.00), 3.506 (3.83), 3.992 (0.85), 4.005 (0.83), 4.025 (0.51), 4.064 (0.53), 4.085 (0.93), 4.098 (0.96), 4.529 (0.72), 4.544 (0.69), 6.293 (0.67), 6.314 (0.69), 6.344 (0.56), 6.998 (1.73), 7.019 (1.70), 7.358 (1.14), 7.376 (1.33), 7.460 (1.97), 7.670 (1.60), 7.687 (0.61), 7.773 (2.32), 7.795 (2.26), 7.816 (0.53), 7.843 (0.91), 7.861 (1.06), 7.951 (0.45), 8.289 (0.43).

### Example 23-B-²²⁷Th

### [²²⁷Th]Thorium-(4S,11S,15S)-4-[(naphthalen-2-yl)methyl]-2,5,13-trioxo-1-[4-(2-{4,7,10-tris[(carboxy-kappaO)methyl]-1,4,7,10-tetraazacyclododecan-1-yl-kappa⁴N¹,N⁴,N⁷,N¹⁰}acetamido)phenyl]-3,6,12,14-tetraazaheptadecane-11,15,17-tricarboxylate

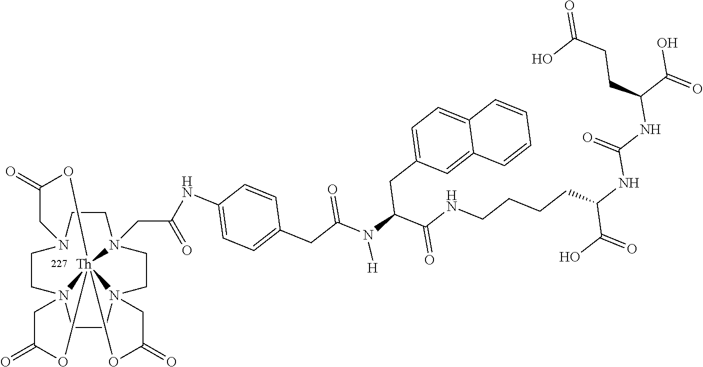

(4S,11S,15S)-4-[(naphthalen-2-yl)methyl]-2,5,13-trioxo-1-(4-{2-[4,7,10-tris(carboxymethyl)-1,4,7,10-tetraazacyclododecan-1-yl]acetamido}phenyl)-3,6,12,14-tetraazaheptadecane-11,15,17-tricarboxylic acid (1.00 mg, 0.965 μmol) was dissolved in 400 mM sodium acetate buffer (pH 5) containing 0.5 mg/mL pABA. Thorium-227 in 400 mM sodium acetate buffer (pH 5) plus 20% EtOH was added giving a specific activity of 0.375 MBq/nmol and a RAC of 3.1 MBq/mL. The mixture was incubated at 95° C. for 60 min. The labelling efficiency was determined to be 75.3% by iTLC.

#24 Linker

Intermediate 104

### tri-tert-butyl (3S,10S,14S)-1-{4-[({[(9H-fluoren-9-yl)methoxy]carbonyl}amino)methyl]phenyl}-3-[(naphthalen-2-yl)methyl]-1,4,12-trioxo-2,5,11,13-tetraazahexadecane-10,14,16-tricarboxylate

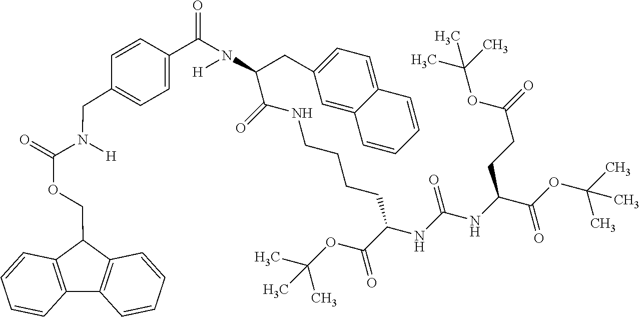 [SMILES: CC(C)(C)OC(=O)CC[C@H](NC(=O)N[C@@H](CCCCNC(=O)[C@H](Cc1ccc2ccccc2c1)NC(=O)c1ccc(CNC(=O)OCC2c3ccccc3-c3ccccc32)cc1)C(=O)OC(C)(C)C)C(=O)OC(C)(C)C]

di-tert-butyl (2S)-2-({[(2S)-6-{[(2S)-2-amino-3-(naphthalen-2-yl)propanoyl]amino}-1-tert-butoxy-1-oxohexan-2-yl]carbamoyl}amino)pentanedioate (290 mg, 95% purity, 402 μmol) and 4-[({[(9H-fluoren-9-yl)methoxy]carbonyl}amino)methyl]benzoic acid (150 mg, 402 μmol) were solubilised in DMF (3.1 ml), 4-methylmorpholine (180 μl, 1.6 mmol, CAS-RN: 109-02-4) and HATU (229 mg, 603 μmol) were added and the mixture was stirred under argon for 1 h at rt. The mixture was evaporated and purified by preparative HPLC (C18, acetonitrile/water with 0.1% formic acid) to give 109 mg (95% purity, 25% yield) of the target compound.

LC-MS (Method 1): Rt=1.62 min; MS (ESIpos): m/z=1041 [M+H]⁺

Intermediate 105

### tri-tert-butyl (3S,10S,14S)-1-[4-(aminomethyl)phenyl]-3-[(naphthalen-2-yl)methyl]-1,4,12-trioxo-2,5,11,13-tetraazahexadecane-10,14,16-tricarboxylate

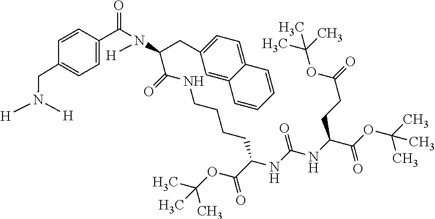 [SMILES: CC(C)(C)OC(=O)CC[C@H](NC(=O)N[C@@H](CCCCNC(=O)[C@H](Cc1ccc2ccccc2c1)NC(=O)c1ccc(CN)cc1)C(=O)OC(C)(C)C)C(=O)OC(C)(C)C]

tri-tert-butyl (3S,10S,14S)-1-{4-[({[(9H-fluoren-9-yl)methoxy]carbonyl}amino)methyl]phenyl}-3-[(naphthalen-2-yl)methyl]-1,4,12-trioxo-2,5,11,13-tetraazahexadecane-10,14,16-tricarboxylate (267 mg, 80% purity, 205 μmol) was solubilised in DMF (2.1 ml), piperidine (390 μl, 2.1 mmol) was added and the mixture was stirred under argon overnight at rt. The mixture was evaporated and purified by preparative HPLC (C18, acetonitrile/water with 0.1% formic acid) to give 56.0 mg (70% purity, 23% yield) of the target compound.

LC-MS (Method 1): Rₜ=1.23 min; MS (ESIpos): m/z=819 [M+H]⁺

¹H-NMR (400 MHz, DMSO-d6) δ[ppm]: 1.232 (0.49), 1.367 (2.07), 1.380 (15.66), 1.527 (0.45), 1.538 (0.56), 1.586 (0.81), 1.592 (0.81), 2.326 (0.76), 2.331 (0.56), 2.518 (3.05), 2.522 (1.92), 2.669 (0.80), 2.673 (0.59), 2.922 (0.90), 3.178 (0.45), 3.205 (0.52), 3.216 (0.59), 3.274 (0.92), 3.288 (1.05), 3.302 (1.19), 3.314 (1.26), 3.327 (1.40), 3.342 (1.48), 5.758 (16.00), 7.399 (0.46), 7.434 (0.46), 7.750 (0.49), 7.771 (0.74), 7.781 (0.60), 7.795 (0.77).

### Example 24-A

### (3S,10S,14S)-1-[4-(aminomethyl)phenyl]-3-[(naphthalen-2-yl)methyl]-1,4,12-trioxo-2,5,11,13-tetraazahexadecane-10,14,16-tricarboxylic acid

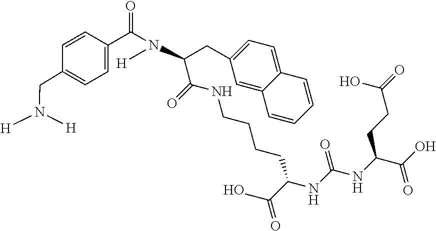 [SMILES: NCc1ccc(C(=O)N[C@@H](Cc2ccc3ccccc3c2)C(=O)NCCCC[C@H](NC(=O)N[C@@H](CCC(=O)O)C(=O)O)C(=O)O)cc1]

tri-tert-butyl (3S,10S,14S)-1-[4-(aminomethyl)phenyl]-3-[(naphthalen-2-yl)methyl]-1,4,12-trioxo-2,5,11,13-tetraazahexadecane-10,14,16-tricarboxylate (25.0 mg, 80% purity, 24.4 μmol) was solubilised in DCM (310 μl), TFA (1.0 ml, 490 μmol) was added and the mixture was stirred under argon overnight at rt. The mixture was evaporated and purified by preparative HPLC (C18, acetonitrile/water with 0.1% formic acid) to give 2.00 mg (90% purity, 11% yield) of the target compound.

LC-MS (Method 1): Rₜ=0.73 min; MS (ESIpos): m/z=650 [M+H]⁺

¹H-NMR (400 MHz, DMSO-d6) δ[ppm]: 0.850 (0.59), 1.170 (0.59), 1.231 (4.32), 1.246 (1.94), 1.255 (2.85), 1.295 (2.06), 1.332 (1.43), 1.347 (2.57), 1.380 (1.66), 1.461 (0.83), 1.608 (1.90), 1.807 (0.59), 2.142 (1.03), 2.246 (0.79), 2.331 (1.74), 2.518 (11.21), 2.522 (7.01), 2.539 (1.07), 2.669 (2.46), 2.673 (1.90), 2.728 (1.62), 2.736 (16.00), 2.887 (1.82), 2.954 (1.54), 3.052 (3.41), 3.064 (3.72), 3.076 (3.72), 3.171 (3.05), 3.197 (3.25), 3.248 (4.40), 3.458 (9.07), 3.504 (8.67), 3.546 (8.79), 3.558 (8.36), 3.570 (7.80), 3.930 (3.33), 4.746 (0.91), 6.159 (0.48), 6.414 (0.59), 7.417 (3.09), 7.432 (4.32), 7.447 (2.73), 7.494 (1.35), 7.514 (1.43), 7.777 (5.70), 7.799 (6.14), 7.830 (1.82), 8.141 (0.79), 8.770 (0.51).

Intermediate 106

### tri-tert-butyl (3S,10S,14S)-3-[(naphthalen-2-yl)methyl]-1,4,12-trioxo-1-[4-({2-[4,7,10-tris(2-tert-butoxy-2-oxoethyl)-1,4,7,10-tetraazacyclododecan-1-yl]acetamido}methyl)phenyl]-2,5,11,13-tetraazahexadecane-10,14,16-tricarboxylate

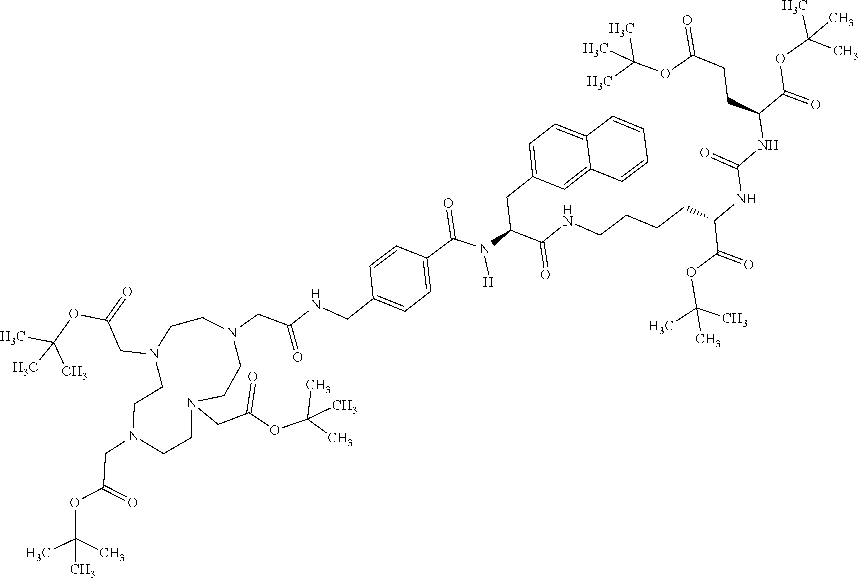 [SMILES: CC(C)(C)OC(=O)CC[C@H](NC(=O)N[C@@H](CCCCNC(=O)[C@H](Cc1ccc2ccccc2c1)NC(=O)c1ccc(CNC(=O)CN2CCN(CC(=O)OC(C)(C)C)CCN(CC(=O)OC(C)(C)C)CCN(CC(=O)OC(C)(C)C)CC2)cc1)C(=O)OC(C)(C)C)C(=O)OC(C)(C)C]

[4,7,10-tris(2-tert-butoxy-2-oxoethyl)-1,4,7,10-tetraazacyclododecan-1-yl]acetic acid (89.6 mg, 156 μmol), [(1H-benzotriazol-1-yl)oxy](dimethylamino)-N,N-dimethylmethaniminium hexafluoridophosphate(1−) (58.6 mg, 155 μmol; CAS-RN:94790-37-1) and N,N-diisopropylethylamine (28 μl, 160 μmol) were stirred in DMF (1 ml) for 10 min at rt. tri-tert-butyl (3S,10S,14S)-1-[4-(aminomethyl)phenyl]-3-[(naphthalen-2-yl)methyl]-1,4,12-trioxo-2,5,11,13-tetraazahexadecane-10,14,16-tricarboxylate (40.0 mg, 80% purity, 39.1 μmol) was added and the mixture was stirred for 5 d at rt. The mixture was evaporated and purified by preparative HPLC (C18, acetonitrile/water with 0.1% formic acid) to give 35.0 mg (85% purity, 55% yield) of the target compound.

LC-MS (Method 1): Rₜ=1.39 min; MS (ESIpos): m/z=1373 [M+H]⁺

¹H-NMR (400 MHz, DMSO-d6) δ[ppm]: 1.337 (2.40), 1.379 (16.00), 1.382 (15.72), 1.399 (4.09), 1.434 (0.72), 1.452 (1.62), 2.518 (1.50), 2.523 (0.95), 2.727 (1.28), 2.888 (1.59), 7.434 (0.44), 7.753 (0.48), 7.762 (0.42), 7.773 (0.62), 7.791 (0.70).

### Example 24-B

### (3S,10S,14S)-3-[(naphthalen-2-yl)methyl]-1,4,12-trioxo-1-[4-({2-[4,7,10-tris(carboxymethyl)-1,4,7,10-tetraazacyclododecan-1-yl]acetamido}methyl)phenyl]-2,5,11,13-tetraazahexadecane-10,14,16-tricarboxylic acid

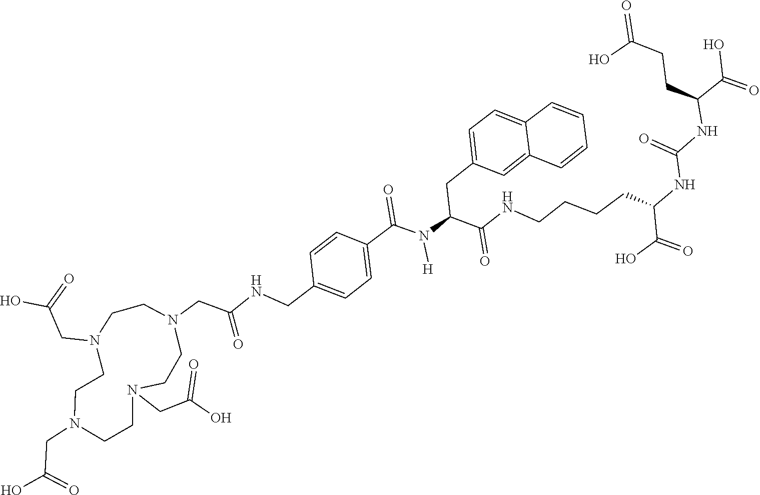 [SMILES: O=C(O)CC[C@H](NC(=O)N[C@@H](CCCCNC(=O)[C@H](Cc1ccc2ccccc2c1)NC(=O)c1ccc(CNC(=O)CN2CCN(CC(=O)O)CCN(CC(=O)O)CCN(CC(=O)O)CC2)cc1)C(=O)O)C(=O)O]

tri-tert-butyl (3S,10S,14S)-3-[(naphthalen-2-yl)methyl]-1,4,12-trioxo-1-[4-({2-[4,7,10-tris(2-tert-butoxy-2-oxoethyl)-1,4,7,10-tetraazacyclododecan-1-yl]acetamido}methyl)phenyl]-2,5,11,13-tetraazahexadecane-10,14,16-tricarboxylate (36.0 mg, 26.2 μmol) was solubilised in DCM (840 μl), TFA (2.0 ml, 26 mmol) was added and the mixture was stirred under argon over the weekend at rt. The mixture was evaporated and purified by preparative HPLC (C18, acetonitrile/water with 0.1% formic acid) to give 6.00 mg (95% purity, 21% yield) of the target compound.

LC-MS (Method 1): Rₜ=0.73 min; MS (ESIpos): m/z=1037 [M+H]⁺

¹H-NMR (400 MHz, DMSO-d6) δ[ppm]: 1.233 (7.44), 1.365 (5.45), 1.485 (2.67), 1.605 (2.50), 1.719 (2.45), 1.885 (2.78), 2.233 (6.28), 2.328 (3.22), 2.668 (6.22), 2.743 (3.50), 2.991 (11.78), 3.047 (11.89), 3.166 (9.98), 3.229 (10.77), 3.418 (16.00), 4.019 (4.01), 4.076 (3.87), 4.295 (4.82), 4.735 (2.56), 6.325 (5.45), 7.331 (5.52), 7.434 (6.59), 7.493 (3.66), 7.513 (3.79), 7.722 (3.98), 7.801 (14.38), 8.107 (3.06), 8.662 (2.74).

### Example 24-B-²²⁷Th

### [²²⁷Th]Thorium (3S,10S,14S)-3-[(naphthalen-2-yl)methyl]-1,4,12-trioxo-1-{4-[(2-{4,7,10-tris[(carboxy-kappaO)methyl]-1,4,7,10-tetraazacyclododecan-1-yl-kappa⁴N¹,N⁴,N⁷,N¹⁰}acetamido)methyl]phenyl}-2,5,11,13-tetraazahexadecane-10,14,16-tricarboxylate

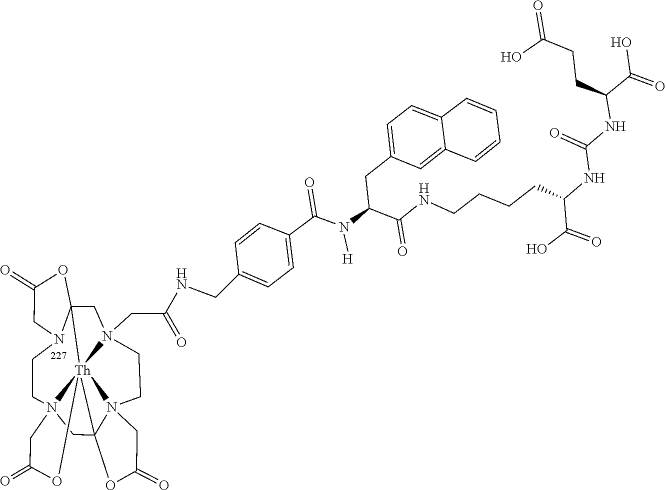

(3S,10S,14S)-3-[(naphthalen-2-yl)methyl]-1,4,12-trioxo-1-[4-({2-[4,7,10-tris(carboxymethyl)-1,4,7,10-tetraazacyclododecan-1-yl]acetamido}methyl)phenyl]-2,5,11,13-tetraazahexadecane-10,14,16-tricarboxylic acid (1.00 mg, 0.965 μmol) was dissolved in 400 mM sodium acetate buffer (pH 5) containing 0.5 mg/mL pABA. Thorium-227 in 400 mM sodium acetate buffer (pH 5) plus 20% EtOH was added giving a specific activity of 0.375 MBq/nmol and a RAC of 3.1 MBq/mL. The mixture was incubated at 95° C. for 60 min. The labelling efficiency was determined to be 83.2% by iTLC.

#25 Linker

Intermediate 107

### 5-[({[(9H-fluoren-9-yl)methoxy]carbonyl}amino)methyl]pyridine-2-carboxylic acid

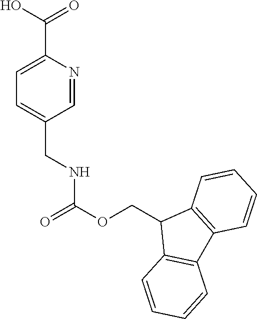 [SMILES: O=C(NCc1ccc(C(=O)O)nc1)OCC1c2ccccc2-c2ccccc21]

5-(aminomethyl)pyridine-2-carboxylic acid (410 mg, 97% purity, 2.61 mmol) was solubilised in 1,4-dioxane (5.0 ml), sodium carbonate (5.8 ml, 2.0 M, 12 mmol) and (9H-fluoren-9-yl)methyl carbonochloridate (751 mg, 2.90 mmol) were added and the mixture was stirred for 2 d at rt. HCl (20 ml, 2.0 M) was added dropwise and extracted with DCM. The organic phase was washed with brine, dried, evaporated and purified by preparative HPLC (C18, acetonitrile/water with 0.1% formic acid) to give 160 mg (95% purity, 16% yield) of the target compound.

LC-MS (Method 1): Rₜ=0.99 min; MS (ESIpos): m/z=375 [M+H]⁺

¹H-NMR (400 MHz, DMSO-d6) δ[ppm]: 1.232 (0.68), 2.336 (1.28), 2.518 (16.00), 2.523 (10.94), 2.678 (1.22), 3.159 (11.07), 3.172 (11.21), 4.082 (1.08), 4.095 (2.16), 4.108 (2.03), 4.121 (0.74), 4.216 (1.22), 4.233 (2.63), 4.249 (1.69), 4.275 (4.79), 4.290 (4.73), 4.368 (7.36), 4.385 (6.21), 7.304 (2.43), 7.320 (5.74), 7.338 (3.71), 7.397 (3.78), 7.416 (6.14), 7.434 (2.84), 7.674 (5.81), 7.693 (5.20), 7.741 (1.96), 7.747 (1.96), 7.761 (2.23), 7.766 (2.23), 7.884 (6.21), 7.903 (5.74), 7.957 (1.42), 7.972 (2.77), 7.993 (4.19), 8.014 (3.24), 8.134 (1.76), 8.573 (3.71), 8.577 (3.71).

Intermediate 108

### tri-tert-butyl (10S,14S)-1-{5-[({[(9H-fluoren-9-yl)methoxy]carbonyl}amino)methyl]pyridin-2-yl}-3-[(naphthalen-2-yl)methyl]-1,4,12-trioxo-2,5,11,13-tetraazahexadecane-10,14,16-tricarboxylate

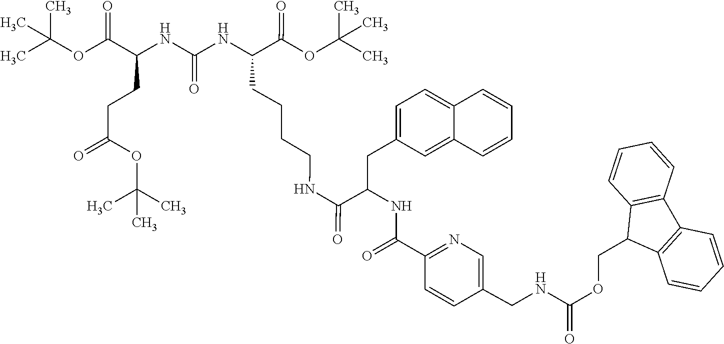 [SMILES: CC(C)(C)OC(=O)CC[C@H](NC(=O)N[C@@H](CCCCNC(=O)C(Cc1ccc2ccccc2c1)NC(=O)c1ccc(CNC(=O)OCC2c3ccccc3-c3ccccc32)cn1)C(=O)OC(C)(C)C)C(=O)OC(C)(C)C]

di-tert-butyl (2S)-2-({[(2S)-6-{[(2S)-2-amino-3-(naphthalen-2-yl)propanoyl]amino}-1-tert-butoxy-1-oxohexan-2-yl]carbamoyl}amino)pentanedioate (129 mg, 85% purity, 160 μmol) and 5-[({[(9H-fluoren-9-yl)methoxy]carbonyl}amino)methyl]pyridine-2-carboxylic acid (70.5 mg, 85% purity, 160 μmol) were solubilised in DMF (1.2 ml), 4-methylmorpholine (79 μl, 640 μmol, CAS-RN: 109-02-4) and HATU (102 mg, 240 μmol) were added and the mixture was stirred under argon at rt. The mixture was diluted with brine and extracted 3 times with DCM. The combined organic layers were dried, evaporated and purified by preparative HPLC (C18, acetonitrile/water with 0.1% formic acid) to give 144 mg (90% purity, 78% yield) of the target compound.

LC-MS (Method 1): Rₜ=1.63 min; MS (ESIpos): m/z=1042 [M+H]⁺

¹H-NMR (400 MHz, DMSO-d6) δ[ppm]: 1.368 (6.92), 1.376 (16.00), 2.518 (1.11), 2.522 (0.71), 2.888 (0.41), 4.362 (0.44), 7.306 (0.41), 7.401 (0.45), 7.420 (0.50), 7.660 (0.41), 7.681 (0.59), 7.764 (0.41), 7.871 (0.42), 7.890 (0.41).

Intermediate 109

### tri-tert-butyl (3S,10S,14S)-1-[5-(aminomethyl)pyridin-2-yl]-3-[(naphthalen-2-yl)methyl]-1,4,12-trioxo-2,5,11,13-tetraazahexadecane-10,14,16-tricarboxylate

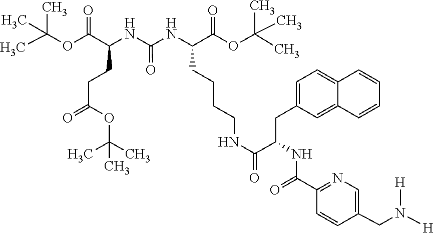 [SMILES: CC(C)(C)OC(=O)CC[C@H](NC(=O)N[C@@H](CCCCNC(=O)[C@H](Cc1ccc2ccccc2c1)NC(=O)c1ccc(CN)cn1)C(=O)OC(C)(C)C)C(=O)OC(C)(C)C]

tri-tert-butyl (3S,10S,14S)-1-{5-[({[(9H-fluoren-9-yl)methoxy]carbonyl}amino)methyl]pyridin-2-yl}-3-[(naphthalen-2-yl)methyl]-1,4,12-trioxo-2,5,11,13-tetraazahexadecane-10,14,16-tricarboxylate (142 mg, 90% purity, 123 μmol) was solubilised in DMF (1.9 ml), piperidine (105 mg, 1.23 mmol) was added and the mixture was stirred under argon for 1 h at rt. The mixture was evaporated and purified by preparative HPLC (C18, acetonitrile/water with 0.1% formic acid) to give 66.0 mg (95% purity, 62% yield) of the target compound.

LC-MS (Method 1): Rₜ=1.21 min; MS (ESIpos): m/z=819 [M+H]⁺

¹H-NMR (400 MHz, DMSO-d6) δ[ppm]: 1.232 (0.42), 1.376 (10.35), 1.380 (16.00), 1.383 (11.88), 1.558 (0.42), 1.626 (0.57), 1.639 (0.69), 2.518 (1.88), 2.523 (1.18), 2.996 (0.83), 3.011 (1.02), 3.024 (0.72), 4.149 (0.88), 7.430 (0.41), 7.441 (0.49), 7.451 (0.43), 7.680 (0.51), 7.758 (0.61), 8.016 (1.00), 8.679 (0.75).

### Example 25-A

### (3S,10S,14S)-1-[5-(aminomethyl)pyridin-2-yl]-3-[(naphthalen-2-yl)methyl]-1,4,12-trioxo-2,5,11,13-tetraazahexadecane-10,14,16-tricarboxylic acid

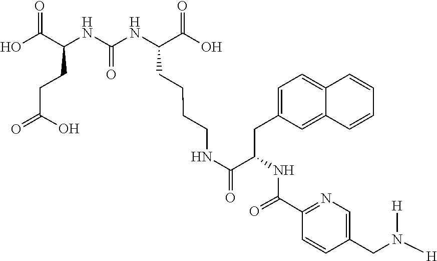 [SMILES: NCc1ccc(C(=O)N[C@@H](Cc2ccc3ccccc3c2)C(=O)NCCCC[C@H](NC(=O)N[C@@H](CCC(=O)O)C(=O)O)C(=O)O)nc1]

tri-tert-butyl (3S,10S,14S)-1-[5-(aminomethyl)pyridin-2-yl]-3-[(naphthalen-2-yl)methyl]-1,4,12-trioxo-2,5,11,13-tetraazahexadecane-10,14,16-tricarboxylate (20.0 mg, 90% purity, 22.0 μmol) was solubilised in DCM (420 μl), TFA (251 mg, 2.20 mmol) was added and the mixture was stirred under argon overnight at rt. The mixture was evaporated and purified by preparative HPLC (C18, acetonitrile/water with 0.1% formic acid) to give 11.9 mg (95% purity, 79% yield) of the target compound.

LC-MS (Method 1): Rₜ=0.72 min; MS (ESIpos): m/z=651 [M+H]⁺

¹H-NMR (400 MHz, DMSO-d6) δ[ppm]: 0.848 (0.61), 1.229 (7.23), 1.244 (7.51), 1.263 (6.62), 1.341 (6.67), 1.360 (7.29), 1.377 (5.11), 1.412 (1.28), 1.432 (2.12), 1.448 (2.85), 1.465 (3.55), 1.484 (2.85), 1.551 (1.98), 1.567 (2.85), 1.582 (3.57), 1.602 (3.16), 1.616 (2.51), 1.682 (0.87), 1.702 (2.09), 1.722 (2.85), 1.737 (3.97), 1.755 (3.83), 1.776 (2.15), 1.797 (3.27), 1.814 (4.13), 1.832 (3.38), 1.847 (2.01), 1.866 (0.92), 2.070 (0.89), 2.199 (7.73), 2.218 (13.26), 2.237 (6.45), 2.322 (1.42), 2.326 (1.82), 2.331 (1.40), 2.522 (4.80), 2.539 (10.86), 2.665 (1.62), 2.669 (1.98), 2.673 (1.62), 2.684 (0.92), 2.725 (9.05), 2.885 (11.06), 3.003 (3.10), 3.019 (4.22), 3.036 (7.04), 3.050 (8.43), 3.062 (7.12), 3.078 (3.99), 3.165 (7.23), 3.180 (3.74), 3.200 (4.33), 3.214 (6.90), 3.235 (7.20), 3.247 (7.20), 3.260 (7.60), 3.281 (4.58), 3.294 (4.10), 3.504 (4.27), 3.950 (9.16), 3.970 (11.59), 3.983 (12.73), 4.003 (13.29), 4.021 (11.42), 4.038 (8.35), 4.107 (14.21), 4.793 (3.60), 4.814 (5.86), 4.828 (6.06), 4.848 (3.38), 6.245 (4.83), 6.264 (4.66), 6.341 (6.20), 6.361 (5.92), 7.358 (7.65), 7.362 (7.57), 7.379 (8.18), 7.382 (8.15), 7.409 (2.60), 7.422 (8.40), 7.426 (13.35), 7.436 (15.22), 7.445 (13.88), 7.450 (8.38), 7.462 (2.60), 7.691 (14.94), 7.749 (7.68), 7.760 (16.00), 7.771 (6.95), 7.781 (11.39), 7.815 (6.87), 7.822 (5.78), 7.838 (6.39), 7.947 (2.01), 7.961 (4.24), 7.981 (11.62), 7.995 (6.09), 8.220 (3.97), 8.235 (7.12), 8.248 (3.63), 8.678 (8.29), 8.691 (9.83), 8.712 (7.96).

Intermediate 110

### tri-tert-butyl (3S,10S,14S)-3-[(naphthalen-2-yl)methyl]-1,4,12-trioxo-1-[5-({2-[4,7,10-tris(2-tert-butoxy-2-oxoethyl)-1,4,7,10-tetraazacyclododecan-1-yl]acetamido}methyl)pyridin-2-yl]-2,5,11,13-tetraazahexadecane-10,14,16-tricarboxylate

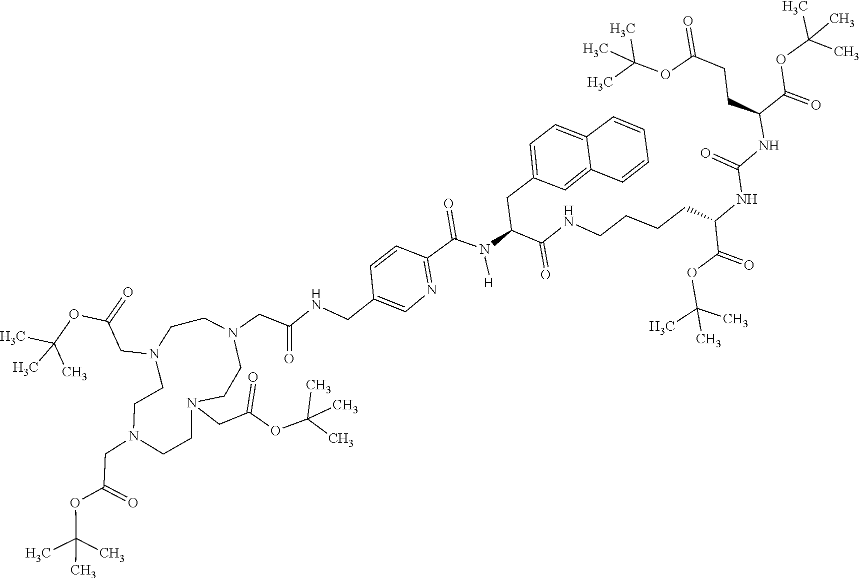 [SMILES: CC(C)(C)OC(=O)CC[C@H](NC(=O)N[C@@H](CCCCNC(=O)[C@H](Cc1ccc2ccccc2c1)NC(=O)c1ccc(CNC(=O)CN2CCN(CC(=O)OC(C)(C)C)CCN(CC(=O)OC(C)(C)C)CCN(CC(=O)OC(C)(C)C)CC2)cn1)C(=O)OC(C)(C)C)C(=O)OC(C)(C)C]

N-[9,12-bis(2-tert-butoxy-2-oxoethyl)-2,2,6-trimethyl-4-oxo-3-oxa-6,9,12-triazatetradecan-14-yl]-N-ethylglycine (119 mg, 202 μmol), [(1H-benzotriazol-1-yl)oxy](dimethylamino)-N,N-dimethylmethaniminium hexafluoridophosphate(1−) (84.1 mg, 200 μmol; CAS-RN:94790-37-1) and N,N-diisopropylethylamine (29 μl, 150 μmol) were stirred in DMF (970 μl) for 10 min at rt. tri-tert-butyl (3S,10S,14S)-1-[5-(aminomethyl)pyridin-2-yl]-3-[(naphthalen-2-yl)methyl]-1,4,12-trioxo-2,5,11,13-tetraazahexadecane-10,14,16-tricarboxylate (46.0 mg, 90% purity, 50.5 μmol) was added and the mixture was stirred for 5 h at rt. The mixture was evaporated and purified by preparative HPLC (C18, acetonitrile/water with 0.1% formic acid) to give 52.0 mg (80% purity, 60% yield) of the target compound.

LC-MS (Method 1): Rₜ=1.37 min; MS (ESIpos): m/z=1374 [M+H]⁺

¹H-NMR (400 MHz, DMSO-d6) δ[ppm]: 1.232 (0.40), 1.313 (2.15), 1.322 (2.42), 1.371 (6.50), 1.380 (16.00), 1.395 (2.85), 1.400 (3.76), 1.435 (3.47), 1.463 (1.08), 1.477 (1.59), 2.518 (1.81), 2.522 (1.16), 2.669 (0.49), 2.888 (0.45).

### Example 25-B

### (3S,10S,14S)-3-[(naphthalen-2-yl)methyl]-1,4,12-trioxo-1-[5-({2-[4,7,10-tris(carboxymethyl)-1,4,7,10-tetraazacyclododecan-1-yl]acetamido}methyl)pyridin-2-yl]-2,5,11,13-tetraazahexadecane-10,14,16-tricarboxylic acid

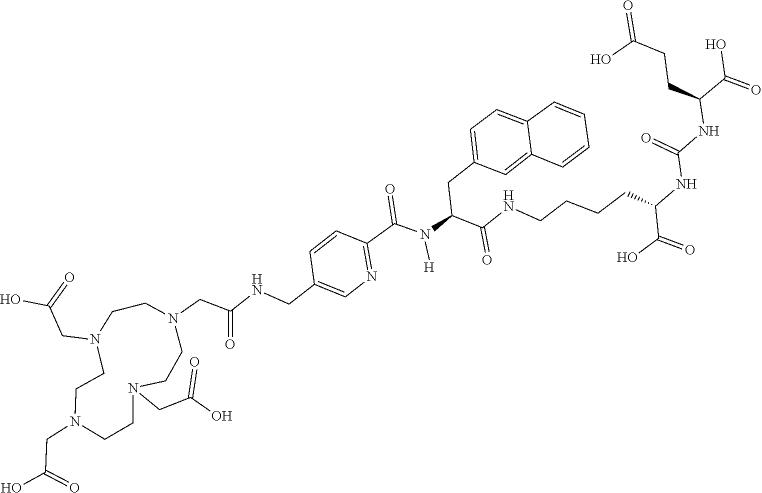 [SMILES: O=C(O)CC[C@H](NC(=O)N[C@@H](CCCCNC(=O)[C@H](Cc1ccc2ccccc2c1)NC(=O)c1ccc(CNC(=O)CN2CCN(CC(=O)O)CCN(CC(=O)O)CCN(CC(=O)O)CC2)cn1)C(=O)O)C(=O)O]

tri-tert-butyl (3S,10S,14S)-3-[(naphthalen-2-yl)methyl]-1,4,12-trioxo-1-[5-({2-[4,7,10-tris(2-tert-butoxy-2-oxoethyl)-1,4,7,10-tetraazacyclododecan-1-yl]acetamido}methyl)pyridin-2-yl]-2,5,11,13-tetraazahexadecane-10,14,16-tricarboxylate (50.0 mg, 80% purity, 29.1 μmol) was solubilised in DCM (740 μl), TFA (670 μl, 8.7 mmol) was added and the mixture was stirred under argon overnight at rt. The mixture was evaporated and purified by preparative HPLC (C18, acetonitrile/water with 0.1% formic acid) to give 7.10 mg (95% purity, 22% yield) of the target compound.

LC-MS (Method 1): Rₜ=0.72 min; MS (ESIpos): m/z=1038 [M+H]⁺

¹H-NMR (400 MHz, DMSO-d6) δ[ppm]: 1.232 (1.76), 1.344 (0.81), 1.462 (0.41), 1.575 (0.41), 1.731 (0.47), 1.751 (0.47), 1.907 (0.61), 2.074 (1.02), 2.084 (0.68), 2.206 (0.95), 2.225 (1.69), 2.244 (0.81), 2.331 (2.92), 2.518 (16.00), 2.522 (10.24), 2.539 (1.02), 2.544 (0.95), 2.576 (0.95), 2.673 (3.05), 2.678 (1.42), 2.910 (1.29), 3.024 (2.58), 3.089 (1.29), 3.440 (1.90), 3.985 (0.54), 4.068 (0.54), 4.382 (0.95), 4.779 (0.54), 4.793 (0.54), 6.298 (0.47), 6.330 (0.54), 6.352 (0.54), 7.370 (0.81), 7.390 (0.88), 7.428 (1.36), 7.437 (1.90), 7.446 (1.36), 7.702 (1.56), 7.773 (1.90), 7.794 (1.69), 7.816 (0.88), 7.839 (1.02), 7.865 (0.61), 7.936 (0.47), 8.142 (1.22), 8.184 (0.68), 8.615 (0.68), 8.689 (0.68), 8.710 (0.68), 8.793 (0.47).

### Example 25-B-²²⁷Th

### [²²⁷Th]Thorium (3S,10S,14S)-3-[(naphthalen-2-yl)methyl]-1,4,12-trioxo-1-{5-[(2-{4,7,10-tris[(carboxy-kappaO)methyl]-1,4,7,10-tetraazacyclododecan-1-yl-kappa⁴N¹,N⁴,N⁷,N¹⁰}acetamido)methyl]pyridin-2-yl}-2,5,11,13-tetraazahexadecane-10,14,16-tricarboxylate

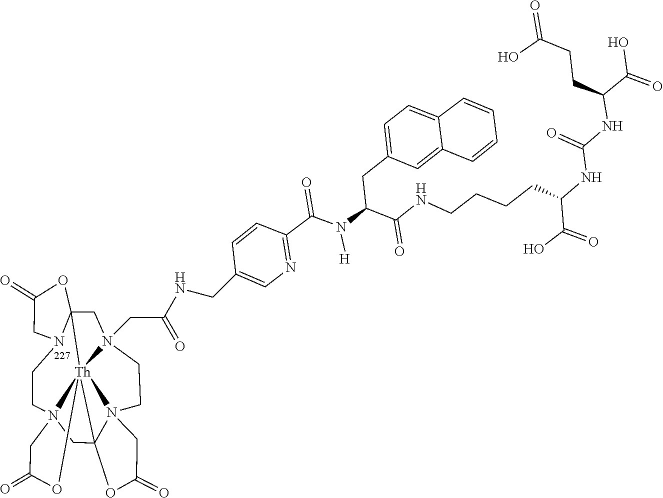

(3S,10S,14S)-3-[(naphthalen-2-yl)methyl]-1,4,12-trioxo-1-[5-({2-[4,7,10-tris(carboxymethyl)-1,4,7,10-tetraazacyclododecan-1-yl]acetamido}methyl)pyridin-2-yl]-2,5,11,13-tetraazahexadecane-10,14,16-tricarboxylic acid (1.00 mg, 0.964 μmol) was dissolved in 400 mM sodium acetate buffer (pH 5) containing 0.5 mg/mL pABA. Thorium-227 in 400 mM sodium acetate buffer (pH 5) plus 20% EtOH was added giving a specific activity of 0.375 MBq/nmol and a RAC of 3.1 MBq/mL. The mixture was incubated at 95° C. for 60 min. The labelling efficiency was determined to be 92.7% by iTLC.

Intermediate 114D

### tri-tert-butyl (3S,10S,14R)-1-{5-[({[(9H-fluoren-9-yl)methoxy]carbonyl}amino)methyl]pyridin-2-yl}-3-[(naphthalen-2-yl)methyl]-1,4,12-trioxo-2,5,11,13-tetraazahexadecane-10,14,16-tricarboxylate

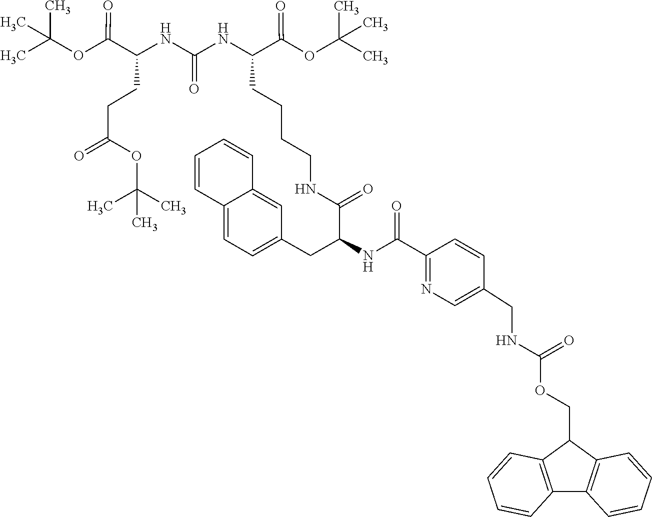 [SMILES: CC(C)(C)OC(=O)CC[C@@H](NC(=O)N[C@@H](CCCCNC(=O)[C@H](Cc1ccc2ccccc2c1)NC(=O)c1ccc(CNC(=O)OCC2c3ccccc3-c3ccccc32)cn1)C(=O)OC(C)(C)C)C(=O)OC(C)(C)C]

A mixture of di-tert-butyl (2R)-2-({[(2S)-6-{[(2S)-2-amino-3-(naphthalen-2-yl)propanoyl]amino}-1-tert-butoxy-1-oxohexan-2-yl]carbamoyl}amino)pentanedioate (584 mg, 94% purity, 801 μmol), 5-[({[(9H-fluoren-9-yl)methoxy]carbonyl}amino)methyl]pyridine-2-carboxylic acid (450 mg, 1.20 mmol), 4-methylmorpholine (260 μl, 2.4 mmol), HATU (457 mg, 1.20 mmol), and DMF (6.2 mL) was stirred at r.t. for 3 h. The mixture was concentrated under reduced pressure and purified by preparative HPLC (C18, acetonitrile/1% TFA) to give the title compound (100 mg, 95% purity, 32% yield).

LC-MS (OA01a01): Rₜ=1.61 min; MS (ESIpos): m/z=1042 [M+H]⁺

1H-NMR (400 MHz, DMSO-d6) delta [ppm]: 1.206 (0.21), 1.231 (0.26), 1.361 (3.25), 1.365 (16.00), 1.389 (9.58), 2.158 (0.17), 2.175 (0.20), 2.179 (0.26), 2.200 (0.24), 2.322 (0.17), 2.326 (0.23), 2.331 (0.17), 2.518 (0.85), 2.522 (0.56), 2.668 (0.23), 2.673 (0.17), 3.202 (0.17), 3.224 (0.25), 3.239 (0.19), 3.955 (0.17), 3.970 (0.19), 4.009 (0.16), 4.016 (0.23), 4.030 (0.22), 4.220 (0.24), 4.237 (0.19), 4.249 (0.37), 4.264 (0.35), 4.363 (0.58), 4.379 (0.48), 5.759 (0.45), 6.293 (0.30), 6.314 (0.55), 6.335 (0.31), 7.287 (0.23), 7.306 (0.52), 7.325 (0.35), 7.357 (0.25), 7.382 (0.40), 7.402 (0.58), 7.421 (0.66), 7.433 (0.39), 7.438 (0.29), 7.444 (0.40), 7.660 (0.51), 7.681 (0.73), 7.727 (0.18), 7.732 (0.19), 7.747 (0.40), 7.752 (0.37), 7.764 (0.56), 7.785 (0.39), 7.814 (0.23), 7.825 (0.22), 7.838 (0.19), 7.871 (0.56), 7.890 (0.53), 7.898 (0.46), 7.918 (0.30), 7.941 (0.25), 8.214 (0.21), 8.481 (0.33), 8.485 (0.33), 8.630 (0.26), 8.652 (0.24).

Intermediate 115D

### tri-tert-butyl (3S,10S,14R)-1-[5-(aminomethyl)pyridin-2-yl]-3-[(naphthalen-2-yl)methyl]-1,4,12-trioxo-2,5,11,13-tetraazahexadecane-10,14,16-tricarboxylate

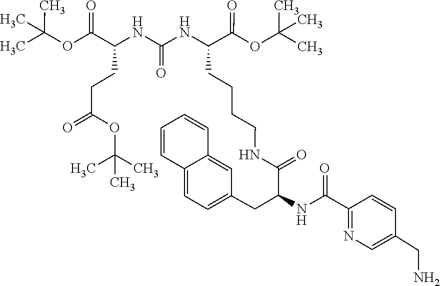 [SMILES: CC(C)(C)OC(=O)CC[C@@H](NC(=O)N[C@@H](CCCCNC(=O)[C@H](Cc1ccc2ccccc2c1)NC(=O)c1ccc(CN)cn1)C(=O)OC(C)(C)C)C(=O)OC(C)(C)C]

A mixture of tri-tert-butyl (3S,10S,14R)-1-{5-[({[(9H-fluoren-9-yl)methoxy]carbonyl}amino)methyl]pyridin-2-yl}-3-[(naphthalen-2-yl)methyl]-1,4,12-trioxo-2,5,11,13-tetraazahexadecane-10,14,16-tricarboxylate (100 mg, 96.0 μmol), piperidine (190 μl, 1.9 mmol), and DMF (2.5 mL) was stirred at r.t. for 3 h. After that the mixture was concentrated under reduced pressure and the residue was purified by preparative HPLC (C18, acetonitrile/1% TFA) to give the title compound (75.0 mg, 95% purity, 91% yield).

LC-MS (Method 1): Rₜ=1.30 min; MS (ESIpos): m/z=820 [M+H]⁺

¹H-NMR (400 MHz, DMSO-d6) δ[ppm]: 1.353 (0.44), 1.371 (16.00), 1.392 (10.35), 2.518 (1.91), 2.523 (1.37), 3.221 (0.40), 3.236 (0.40), 3.339 (1.51), 3.857 (1.52), 6.318 (0.46), 7.429 (0.42), 7.438 (0.56), 7.448 (0.47), 7.681 (0.50), 7.764 (0.62), 7.915 (1.43), 7.919 (1.22), 8.235 (1.71), 8.581 (0.58).

### Example 25-A-D

### (3S,10S,14R)-1-[5-(aminomethyl)pyridin-2-yl]-3-[(naphthalen-2-yl)methyl]-1,4,12-trioxo-2,5,11,13-tetraazahexadecane-10,14,16-tricarboxylic acid

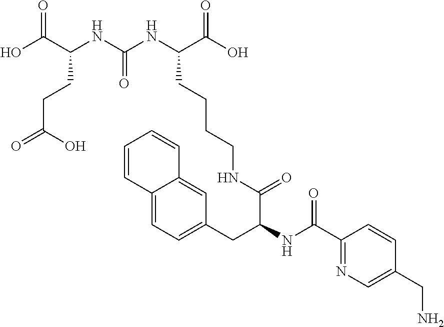 [SMILES: NCc1ccc(C(=O)N[C@@H](Cc2ccc3ccccc3c2)C(=O)NCCCC[C@H](NC(=O)N[C@H](CCC(=O)O)C(=O)O)C(=O)O)nc1]

A mixture of tri-tert-butyl (3S,10S,14R)-1-[5-(aminomethyl)pyridin-2-yl]-3-[(naphthalen-2-yl)methyl]-1,4,12-trioxo-2,5,11,13-tetraazahexadecane-10,14,16-tricarboxylate (10.0 mg, 12.2 μmol), TFA (300 μL), and DCM (1.0 mL) was stirred at r.t. for 2 h. After that the mixture was concentrated under reduced pressure and purified by preparative HPLC (C18, acetonitrile/1% formic acid) to give the title compound (6.20 mg, 95% purity, 74% yield).

LC-MS (Method 1): Rₜ=0.74 min; MS (ESIpos): m/z=652 [M+H]⁺

1H-NMR (400 MHz, DMSO-d6) delta [ppm]: 1.233 (2.32), 1.253 (1.93), 1.271 (0.90), 1.337 (1.93), 1.356 (2.17), 1.374 (1.40), 1.439 (0.63), 1.458 (0.85), 1.473 (1.09), 1.492 (0.85), 1.548 (0.77), 1.563 (0.98), 1.583 (0.92), 1.597 (0.53), 1.617 (0.44), 1.636 (0.57), 1.650 (0.94), 1.669 (1.16), 1.684 (0.88), 1.703 (0.44), 1.732 (0.39), 1.753 (1.05), 1.770 (1.23), 1.787 (0.92), 1.803 (0.68), 1.824 (0.31), 2.073 (16.00), 2.108 (0.39), 2.121 (0.53), 2.145 (1.38), 2.164 (1.55), 2.173 (1.53), 2.193 (2.21), 2.214 (1.14), 2.231 (0.88), 2.252 (0.35), 2.518 (4.36), 2.522 (2.91), 2.539 (1.90), 2.995 (0.85), 3.010 (1.31), 3.027 (1.90), 3.052 (2.06), 3.066 (1.99), 3.083 (1.44), 3.099 (1.05), 3.180 (1.49), 3.200 (1.66), 3.213 (2.56), 3.234 (2.69), 3.247 (2.65), 3.260 (2.82), 3.280 (1.93), 3.294 (1.82), 3.637 (3.02), 3.933 (2.30), 3.953 (3.00), 3.965 (3.44), 3.984 (3.48), 4.000 (2.95), 4.029 (8.05), 4.788 (1.01), 4.803 (1.27), 4.809 (1.73), 4.823 (1.75), 4.843 (0.94), 6.246 (1.12), 6.262 (1.09), 6.452 (1.82), 6.472 (1.75), 7.357 (2.43), 7.361 (2.45), 7.379 (2.56), 7.382 (2.65), 7.404 (0.46), 7.409 (0.81), 7.421 (2.65), 7.426 (4.55), 7.435 (5.10), 7.444 (4.73), 7.449 (2.78), 7.461 (0.85), 7.691 (4.64), 7.751 (2.39), 7.760 (5.17), 7.775 (2.21), 7.781 (3.68), 7.814 (2.17), 7.822 (1.73), 7.837 (1.99), 7.941 (1.01), 7.961 (6.54), 7.989 (0.63), 8.197 (1.14), 8.211 (2.85), 8.225 (1.12), 8.653 (3.94), 8.708 (2.74), 8.729 (2.63).

### Example 25-C-D

### (3S,10S,14R)-1-[5-({2-[4-({2-[{3-[bis(2-{[1-(carboxymethyl)-3-hydroxy-6-methyl-2-oxo-1,2-dihydropyridine-4-carbonyl]amino}ethyl)amino]propyl}(2-{[1-(carboxymethyl)-3-hydroxy-6-methyl-2-oxo-1,2-dihydropyridine-4-carbonyl]amino}ethyl)amino]ethyl}carbamoyl)-3-hydroxy-6-methyl-2-oxopyridin-1(2H)-yl]acetamido}methyl)pyridin-2-yl]-3-[(naphthalen-2-yl)methyl]-1,4,12-trioxo-2,5,11,13-tetraazahexadecane-10,14,16-tricarboxylic acid

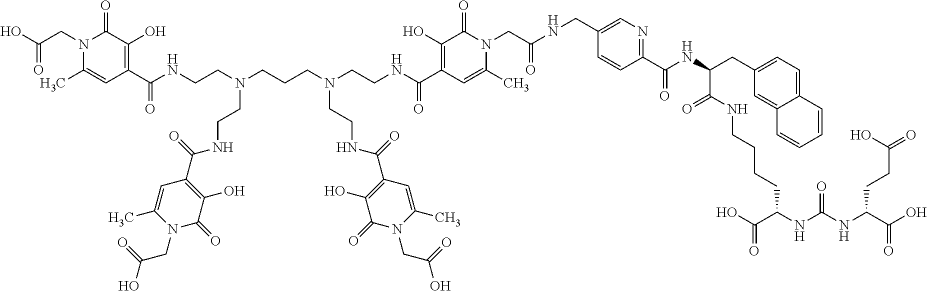 [SMILES: Cc1cc(C(=O)NCCN(CCCN(CCNC(=O)c2cc(C)n(CC(=O)O)c(=O)c2O)CCNC(=O)c2cc(C)n(CC(=O)NCc3ccc(C(=O)N[C@@H](Cc4ccc5ccccc5c4)C(=O)NCCCC[C@H](NC(=O)N[C@H](CCC(=O)O)C(=O)O)C(=O)O)nc3)c(=O)c2O)CCNC(=O)c2cc(C)n(CC(=O)O)c(=O)c2O)c(O)c(=O)n1CC(=O)O]

2,2′-{({3-[(2-{[1-(2-{[(6-{[(7R,11S,18S)-7,11-bis(tert-butoxycarbonyl)-2,2-dimethyl-19-(naphthalen-2-yl)-4,9,17-trioxo-3-oxa-8,10,16-triazanonadecan-18-yl]carbamoyl}pyridin-3-yl)methyl]amino}-2-oxoethyl)-3-hydroxy-6-methyl-2-oxo-1,2-dihydropyridine-4-carbonyl]amino}ethyl)(2-{[1-(carboxymethyl)-3-hydroxy-6-methyl-2-oxopyridine-4(2H)-carbonyl]amino}ethyl)amino]propyl}azanediyl)bis[(ethane-2,1-diyl)carbamoyl(3-hydroxy-6-methyl-2-oxopyridine-4,1(2H)-diyl)]}diacetic acid (2.40 mg, 1.27 μmol) is treated with 90% TFA in water (1 mL). Water (15 mL) is added and solution lyophilised affording 2.3 mg of the target compound.

LC-MS (Method K, gradient: 10-50% B over 3 min): Rₜ=2.13 min; MS (ESIpos): m/z=1715.8 [M+H]⁺

### Example 25-C-D-²²⁷Th

### (3S,10S,14R)-1-[5-({2-[4-({2-[{3-[bis(2-{[1-(carboxymethyl)-3-(hydroxy-kappaO)-6-methyl-2-oxo-1,2-dihydropyridine-4-carbonyl]amino}ethyl)amino]propyl}(2-{[1-(carboxymethyl)-3-(hydroxy-kappaO)-6-methyl-2-oxo-1,2-dihydropyridine-4-carbonyl]amino}ethyl)amino]ethyl}carbamoyl)-3-(hydroxy-kappaO)-6-methyl-2-oxopyridin-1(2H)-yl]acetamido}methyl)pyridin-2-yl]-3-[(naphthalen-2-yl)methyl]-1,4,12-trioxo-2,5,11,13-tetraazahexadecane-10,14,16-tricarboxylato(4−)(²²⁷Th)Thoriuum

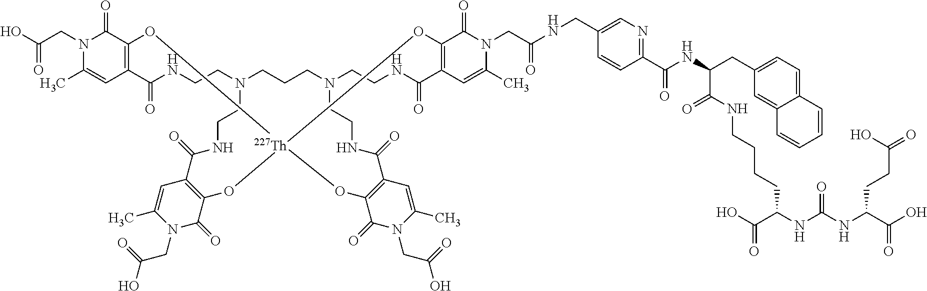

(3S,10S,14R)-1-[5-({2-[4-({2-[{3-[bis(2-{[1-(carboxymethyl)-3-hydroxy-6-methyl-2-oxo-1,2-dihydropyridine-4-carbonyl]amino}ethyl)amino]propyl}(2-{[1-(carboxymethyl)-3-hydroxy-6-methyl-2-oxo-1,2-dihydropyridine-4-carbonyl]amino}ethyl)amino]ethyl}carbamoyl)-3-hydroxy-6-methyl-2-oxopyridin-1(2H)-yl]acetamido}methyl)pyridin-2-yl]-3-[(naphthalen-2-yl)methyl]-1,4,12-trioxo-2,5,11,13-tetraazahexadecane-10,14,16-tricarboxylic acid (4 μg dissolved in 147 μL 30 mM citrate buffer (pH 5.5) containing 0.5 mg/mL pABA was mixed with thorium-227 in 0.5 M HCl (2 μL) at 0.3 MBq/nmol specific activity and RAC of 4.6 MBq/mL. 1M carbonate buffer pH 9 (15 μL) was added and mixture incubated for 60 min. The labelling efficiency was determined to be 97% by iTLC.

#26 Linker

Intermediate 111

### 6-[({[(9H-fluoren-9-yl)methoxy]carbonyl}amino)methyl]pyridine-3-carboxylic acid

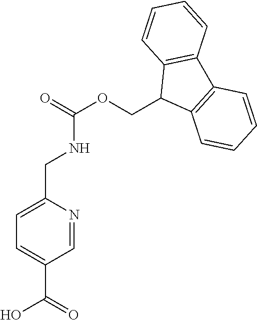 [SMILES: O=C(NCc1ccc(C(=O)O)cn1)OCC1c2ccccc2-c2ccccc21]

6-(aminomethyl)pyridine-3-carboxylic acid (140.0 mg, 920 μmol) was solubilised in 1,4-dioxane (1.1 ml, 13 mmol), Na2CO3 (4.60 ml, 2.0 M, 9.2 mmol) and (9H-fluoren-9-yl)methyl carbonochloridate (261.8 mg, 1.01 mmol) were added. The mixture was stirred over the weekend at rt under nitrogen atmosphere. The mixture was diluted with brine and extracted with DCM.

The organic phase was dried, evaporated and purified by preparative HPLC (C18, acetonitrile/water with 0.1% formic acid) to give 139 mg (85% purity, 40% yield) of the target compound.

LC-MS (Method 1): Rt=1.10 min; MS (ESIpos): m/z=375 [M+H]⁺

¹H-NMR (400 MHz, DMSO-d6) δ[ppm]: 1.232 (0.84), 2.331 (1.80), 2.518 (8.29), 2.523 (5.45), 2.728 (13.57), 2.737 (5.28), 2.888 (16.00), 3.053 (0.46), 3.065 (0.59), 3.077 (0.59), 3.546 (0.54), 3.558 (0.59), 3.571 (0.50), 3.719 (3.64), 3.732 (4.31), 3.737 (4.65), 3.749 (4.06), 3.981 (1.93), 3.999 (3.48), 4.016 (1.51), 4.234 (1.21), 4.251 (2.64), 4.267 (1.68), 4.330 (4.73), 4.345 (4.69), 4.374 (6.12), 4.391 (4.65), 5.069 (1.63), 5.082 (3.06), 5.095 (1.47), 7.205 (0.50), 7.280 (3.64), 7.282 (4.06), 7.298 (9.97), 7.301 (9.21), 7.317 (9.26), 7.320 (8.46), 7.337 (5.91), 7.359 (7.25), 7.379 (7.75), 7.398 (3.64), 7.408 (3.43), 7.427 (5.36), 7.445 (2.30), 7.533 (0.42), 7.552 (0.42), 7.656 (7.58), 7.674 (6.91), 7.711 (5.40), 7.730 (4.77), 7.799 (0.71), 7.818 (0.71), 7.849 (8.29), 7.868 (7.62), 7.896 (5.70), 7.914 (5.15), 7.950 (2.14), 7.974 (1.26), 7.990 (2.55), 8.006 (1.17), 8.211 (2.30), 8.217 (2.35), 8.232 (2.14), 8.237 (2.09), 8.975 (4.61), 8.979 (4.65), 13.364 (0.50).

Intermediate 112

### tri-tert-butyl (3S,10S,14S)-1-{6-[({[(9H-fluoren-9-yl)methoxy]carbonyl}amino)methyl]pyridin-3-yl}-3-[(naphthalen-2-yl)methyl]-1,4,12-trioxo-2,5,11,13-tetraazahexadecane-10,14,16-tricarboxylate

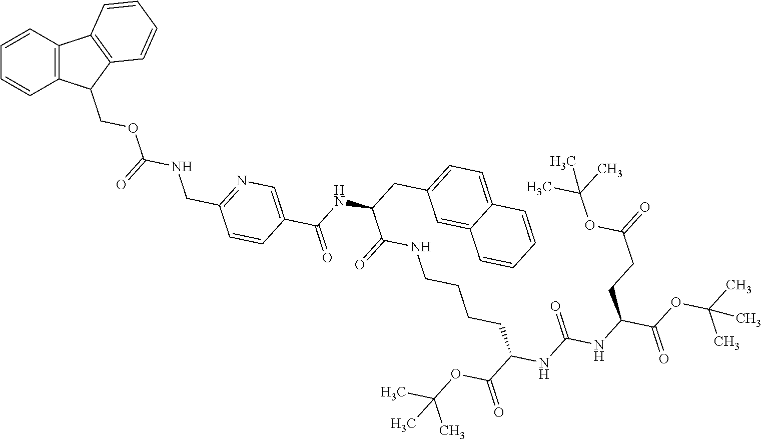 [SMILES: CC(C)(C)OC(=O)CC[C@H](NC(=O)N[C@@H](CCCCNC(=O)[C@H](Cc1ccc2ccccc2c1)NC(=O)c1ccc(CNC(=O)OCC2c3ccccc3-c3ccccc32)nc1)C(=O)OC(C)(C)C)C(=O)OC(C)(C)C]

di-tert-butyl (2S)-2-({[(2S)-6-{[(2S)-2-amino-3-(naphthalen-2-yl)propanoyl]amino}-1-tert-butoxy-1-oxohexan-2-yl]carbamoyl}amino)pentanedioate (122 mg, 178 μmol) and 6-[({[(9H-fluoren-9-yl)methoxy]carbonyl}amino)methyl]pyridine-3-carboxylic acid (66.7 mg, 178 μmol) were solubilised in DMF (4.1 ml), 4-methylmorpholine (78 μl, 710 μmol, CAS-RN: 109-02-4) and HATU (102 mg, 267 μmol) were added and the mixture was stirred under argon at rt. The mixture was diluted with brine and extracted with DCM. The organic phase was dried, evaporated and purified by preparative HPLC (C18, acetonitrile/water with 0.1% formic acid) to give 94.0 mg (85% purity, 43% yield) of the target compound.

LC-MS (Method 1): Rₜ=1.60 min; MS (ESIpos): m/z=1042 [M+H]⁺

¹H-NMR (400 MHz, DMSO-d6) δ[ppm]: 1.232 (0.46), 1.377 (13.29), 1.381 (16.00), 2.518 (2.09), 2.523 (1.36), 2.728 (0.46), 2.888 (0.57), 4.356 (0.49), 7.418 (0.59), 7.435 (0.49), 7.791 (0.42), 7.801 (0.52), 7.813 (0.49), 7.888 (0.47), 7.907 (0.44).

Intermediate 113

### tri-tert-butyl (3S,10S,14S)-1-[6-(aminomethyl)pyridin-3-yl]-3-[(naphthalen-2-yl)methyl]-1,4,12-trioxo-2,5,11,13-tetraazahexadecane-10,14,16-tricarboxylate

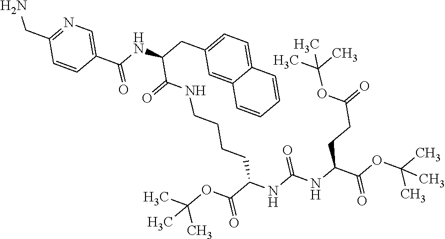 [SMILES: CC(C)(C)OC(=O)CC[C@H](NC(=O)N[C@@H](CCCCNC(=O)[C@H](Cc1ccc2ccccc2c1)NC(=O)c1ccc(CN)nc1)C(=O)OC(C)(C)C)C(=O)OC(C)(C)C]

tri-tert-butyl (3S,10S,14S)-1-{6-[({[(9H-fluoren-9-yl)methoxy]carbonyl}amino)methyl]pyridin-3-yl}-3-[(naphthalen-2-yl)methyl]-1,4,12-trioxo-2,5,11,13-tetraazahexadecane-10,14,16-tricarboxylate (93.0 mg, 85% purity, 75.9 μmol) was solubilised in DMF (1.8 ml), piperidine (32.3 mg, 380 μmol) was added and the mixture was stirred under argon for 2 h at rt. The mixture was evaporated and purified by preparative HPLC (C18, acetonitrile/water with 0.1% formic acid) to give 47.0 mg (85% purity, 64% yield) of the target compound.

LC-MS (Method 1): Rₜ=1.24 min; MS (ESIpos): m/z=820 [M+H]⁺

¹H-NMR (400 MHz, DMSO-d6) δ[ppm]: 1.380 (13.40), 1.385 (16.00), 2.518 (1.27), 2.523 (0.79), 2.888 (0.48), 3.984 (0.69), 7.441 (0.42), 7.790 (0.45), 7.801 (0.62), 7.810 (0.45), 8.867 (0.56).

### Example 26-A

### (3S,10S,14S)-1-[6-(aminomethyl)pyridin-3-yl]-3-[(naphthalen-2-yl)methyl]-1,4,12-trioxo-2,5,11,13-tetraazahexadecane-10,14,16-tricarboxylic acid

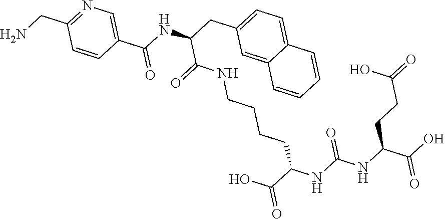 [SMILES: NCc1ccc(C(=O)N[C@@H](Cc2ccc3ccccc3c2)C(=O)NCCCC[C@H](NC(=O)N[C@@H](CCC(=O)O)C(=O)O)C(=O)O)cn1]

tri-tert-butyl (3S,10S,14S)-1-[6-(aminomethyl)pyridin-3-yl]-3-[(naphthalen-2-yl)methyl]-1,4,12-trioxo-2,5,11,13-tetraazahexadecane-10,14,16-tricarboxylate (15.0 mg, 18.3 μmol) was solubilised in DCM (350 μl), TFA (20.9 mg, 183 μmol) was added and the mixture was stirred under argon overnight at rt. The mixture was evaporated and purified by preparative HPLC (C18, acetonitrile/water with 0.1% formic acid) to give 10.0 mg (95% purity, 80% yield) of the target compound.

LC-MS (Method 1): Rₜ=0.71 min; MS (ESIpos): m/z=651 [M+H]⁺

¹H-NMR (400 MHz, DMSO-d6) δ[ppm]: 1.232 (1.16), 1.272 (1.23), 1.290 (1.16), 1.360 (1.10), 1.378 (1.16), 1.459 (0.65), 1.481 (0.52), 1.594 (0.84), 1.613 (0.97), 1.631 (0.84), 1.789 (0.45), 1.812 (0.58), 1.829 (0.52), 1.846 (0.39), 1.906 (0.52), 2.084 (3.74), 2.159 (0.65), 2.178 (0.71), 2.236 (0.52), 2.259 (0.77), 2.279 (0.52), 2.295 (0.45), 2.332 (2.71), 2.336 (1.29), 2.518 (16.00), 2.522 (10.26), 2.673 (2.71), 2.678 (1.23), 3.032 (0.71), 3.048 (1.03), 3.063 (1.16), 3.079 (1.16), 3.095 (1.16), 3.112 (1.03), 3.128 (1.29), 3.160 (1.61), 3.186 (1.81), 3.269 (4.39), 3.973 (1.42), 3.986 (1.42), 4.112 (5.48), 4.742 (0.45), 4.754 (0.52), 4.767 (0.65), 4.775 (0.65), 6.167 (0.52), 6.399 (0.97), 6.420 (0.90), 7.408 (0.45), 7.421 (1.29), 7.425 (1.23), 7.439 (2.13), 7.458 (1.23), 7.471 (0.52), 7.491 (1.74), 7.511 (2.19), 7.529 (1.42), 7.785 (3.16), 7.806 (2.71), 7.816 (3.81), 7.839 (1.23), 8.171 (1.42), 8.176 (1.48), 8.183 (1.03), 8.191 (1.81), 8.196 (1.68), 8.206 (1.16), 8.903 (2.19), 8.907 (2.13), 9.063 (0.90), 9.084 (0.90).

Intermediate 114

### tri-tert-butyl (3S,10S,14S)-3-[(naphthalen-2-yl)methyl]-1,4,12-trioxo-1-[6-({2-[4,7,10-tris(2-tert-butoxy-2-oxoethyl)-1,4,7,10-tetraazacyclododecan-1-yl]acetamido}methyl)pyridin-3-yl]-2,5,11,13-tetraazahexadecane-10,14,16-tricarboxylate

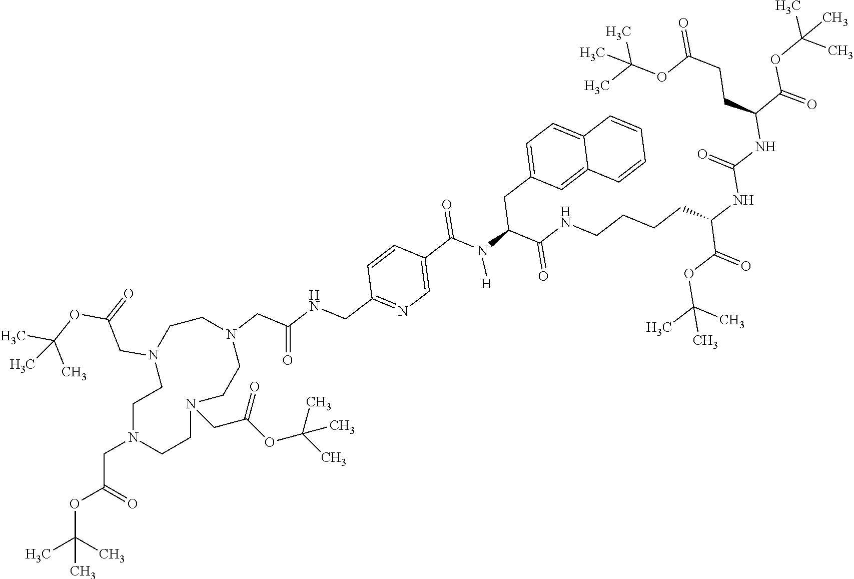 [SMILES: CC(C)(C)OC(=O)CC[C@H](NC(=O)N[C@@H](CCCCNC(=O)[C@H](Cc1ccc2ccccc2c1)NC(=O)c1ccc(CNC(=O)CN2CCN(CC(=O)OC(C)(C)C)CCN(CC(=O)OC(C)(C)C)CCN(CC(=O)OC(C)(C)C)CC2)nc1)C(=O)OC(C)(C)C)C(=O)OC(C)(C)C]

N-[9,12-bis(2-tert-butoxy-2-oxoethyl)-2,2,6-trimethyl-4-oxo-3-oxa-6,9,12-triazatetradecan-14-yl]-N-ethylglycine (73.3 mg, 125 μmol), [(1H-benzotriazol-1-yl)oxy](dimethylamino)-N,N-dimethylmethaniminium hexafluoridophosphate(1−) (46.6 mg, 123 μmol; CAS-RN:94790-37-1) and N,N-diisopropylethylamine (16 μl, 93 μmol) were stirred in DMF (720 μl) for 10 min at rt. tri-tert-butyl (3S,10S,14S)-1-[6-(aminomethyl)pyridin-3-yl]-3-[(naphthalen-2-yl)methyl]-1,4,12-trioxo-2,5,11,13-tetraazahexadecane-10,14,16-tricarboxylate (30.0 mg, 85% purity, 31.1 μmol) was added and the mixture was stirred for 3 h at rt. The mixture was evaporated and purified by preparative HPLC (C18, acetonitrile/water with 0.1% formic acid) to give 30.0 mg (70% purity, 49% yield) of the target compound.

LC-MS (Method 1): Rₜ=1.36 min; MS (ESIpos): m/z=1374 [M+H]⁺

¹H-NMR (400 MHz, DMSO-d6) δ[ppm]: 1.256 (0.42), 1.277 (2.27), 1.295 (2.44), 1.380 (12.53), 1.385 (16.00), 1.414 (0.66), 1.433 (3.11), 1.462 (0.47), 2.518 (1.54), 2.523 (0.98), 2.686 (1.88), 2.727 (5.20), 2.888 (6.62), 7.438 (0.43), 7.799 (0.56), 7.950 (0.84).

### Example 26-B

### (3S,10S,14S)-3-[(naphthalen-2-yl)methyl]-1,4,12-trioxo-1-[6-({2-[4,7,10-tris(carboxymethyl)-1,4,7,10-tetraazacyclododecan-1-yl]acetamido}methyl)pyridin-3-yl]-2,5,11,13-tetraazahexadecane-10,14,16-tricarboxylic acid

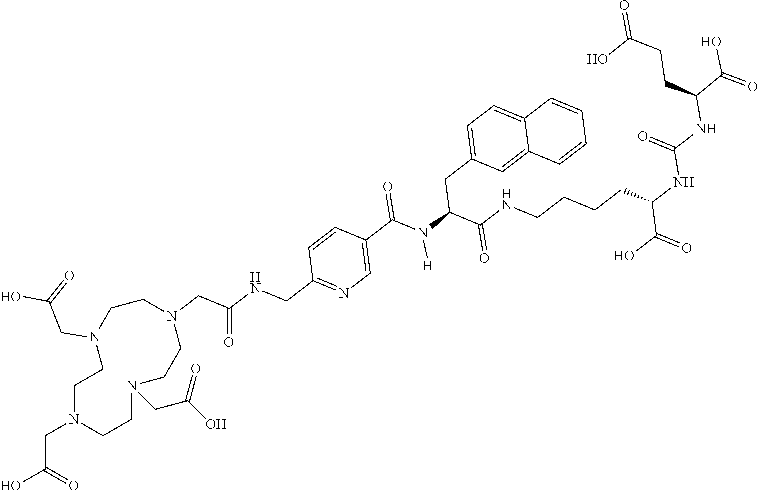 [SMILES: O=C(O)CC[C@H](NC(=O)N[C@@H](CCCCNC(=O)[C@H](Cc1ccc2ccccc2c1)NC(=O)c1ccc(CNC(=O)CN2CCN(CC(=O)O)CCN(CC(=O)O)CCN(CC(=O)O)CC2)nc1)C(=O)O)C(=O)O]

tri-tert-butyl (3S,10S,14S)-3-[(naphthalen-2-yl)methyl]-1,4,12-trioxo-1-[6-({2-[4,7,10-tris(2-tert-butoxy-2-oxoethyl)-1,4,7,10-tetraazacyclododecan-1-yl]acetamido}methyl)pyridin-3-yl]-2,5,11,13-tetraazahexadecane-10,14,16-tricarboxylate (28.0 mg, 70% purity, 14.3 μmol) was solubilised in DCM (460 μl), TFA (16.3 mg, 143 μmol) was added and the mixture was stirred under argon overnight at rt. The mixture was evaporated and purified by preparative HPLC (C18, acetonitrile/water with 0.1% formic acid) to give 14.0 mg (95% purity, 90% yield) of the target compound.

LC-MS (Method 1): Rₜ=0.68 min; MS (ESIpos): m/z=1038 [M+H]⁺

¹H-NMR (400 MHz, DMSO-d6) δ[ppm]: 1.137 (1.27), 1.233 (1.54), 1.255 (1.54), 1.274 (1.34), 1.352 (1.41), 1.371 (1.41), 1.473 (0.60), 1.489 (0.67), 1.509 (0.60), 1.604 (0.67), 1.625 (0.67), 1.670 (0.47), 1.684 (0.60), 1.706 (0.74), 1.720 (0.54), 1.897 (0.67), 1.907 (0.94), 1.914 (0.74), 2.115 (0.60), 2.200 (0.94), 2.217 (1.54), 2.237 (1.61), 2.258 (0.87), 2.331 (2.81), 2.336 (1.34), 2.518 (16.00), 2.522 (10.64), 2.539 (0.94), 2.673 (2.95), 2.678 (1.41), 2.729 (0.80), 2.796 (1.34), 2.888 (1.00), 2.983 (6.56), 3.058 (1.81), 3.102 (1.14), 3.135 (1.47), 3.162 (1.41), 3.491 (2.95), 3.517 (3.55), 3.995 (0.54), 4.015 (1.00), 4.028 (1.00), 4.048 (0.47), 4.066 (0.60), 4.086 (1.14), 4.099 (1.21), 4.120 (0.60), 4.409 (1.74), 4.421 (1.67), 4.744 (0.67), 6.292 (1.61), 6.304 (1.87), 6.313 (1.74), 6.325 (1.61), 7.374 (1.34), 7.395 (1.41), 7.405 (0.74), 7.409 (0.74), 7.422 (1.61), 7.426 (1.47), 7.442 (2.41), 7.457 (1.41), 7.461 (1.54), 7.474 (0.67), 7.497 (1.47), 7.521 (1.67), 7.793 (3.75), 7.812 (4.75), 7.840 (1.47), 8.039 (0.87), 8.060 (0.80), 8.133 (1.61), 8.193 (1.21), 8.763 (0.94), 8.815 (1.87), 8.933 (0.67), 8.951 (0.60).

### Example 26-B-²²⁷Th

### [²²⁷Th]Thorium (3S,10S,14S)-3-[(naphthalen-2-yl)methyl]-1,4,12-trioxo-1-{6-[(2-{4,7,10-tris[(carboxy-kappaO)methyl]-1,4,7,10-tetraazacyclododecan-1-yl-kappa⁴N¹,N⁴,N⁷,N¹⁰}acetamido)methyl]pyridin-3-yl}-2,5,11,13-tetraazahexadecane-10,14,16-tricarboxylate

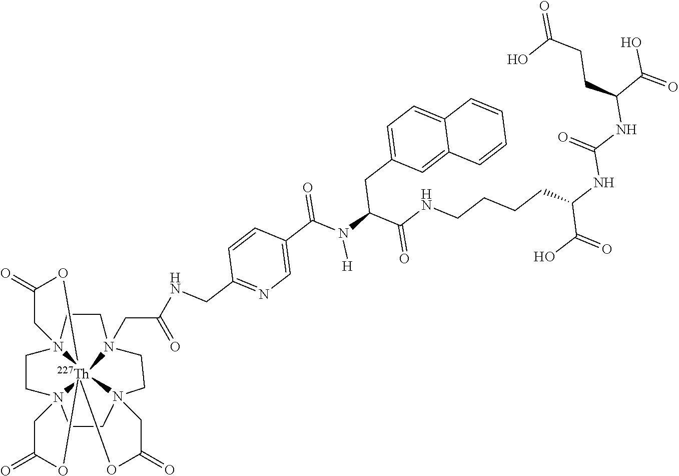

(3S,10S,14S)-3-[(naphthalen-2-yl)methyl]-1,4,12-trioxo-1-[6-({2-[4,7,10-tris(carboxymethyl)-1,4,7,10-tetraazacyclododecan-1-yl]acetamido}methyl)pyridin-3-yl]-2,5,11,13-tetraazahexadecane-10,14,16-tricarboxylic acid (1.00 mg, 0.964 μmol) was dissolved in 400 mM sodium acetate buffer (pH 5) containing 0.5 mg/mL pABA. Thorium-227 in 400 mM sodium acetate buffer (pH 5) plus 20% EtOH was added giving a specific activity of 0.375 MBq/nmol and a RAC of 3.1 MBq/mL. The mixture was incubated at 95° C. for 60 min. The labelling efficiency was determined to be 87% by iTLC.

#27 Linker

Intermediate 115

### tri-tert-butyl (3S,10S,14S)-1-{3-[({[(9H-fluoren-9-yl)methoxy]carbonyl}amino)methyl]phenyl}-3-[(naphthalen-2-yl)methyl]-1,4,12-trioxo-2,5,11,13-tetraazahexadecane-10,14,16-tricarboxylate

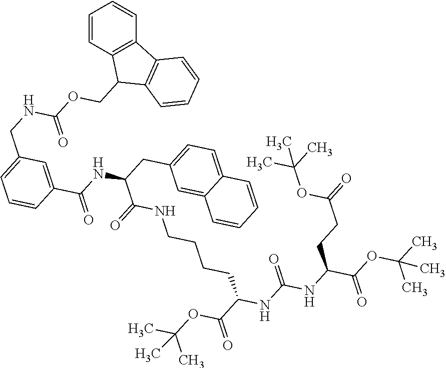 [SMILES: CC(C)(C)OC(=O)CC[C@H](NC(=O)N[C@@H](CCCCNC(=O)[C@H](Cc1ccc2ccccc2c1)NC(=O)c1cccc(CNC(=O)OCC2c3ccccc3-c3ccccc32)c1)C(=O)OC(C)(C)C)C(=O)OC(C)(C)C]

di-tert-butyl (2S)-2-({[(2S)-6-{[(2S)-2-amino-3-(naphthalen-2-yl)propanoyl]amino}-1-tert-butoxy-1-oxohexan-2-yl]carbamoyl}amino)pentanedioate (136 mg, 90% purity, 179 μmol) and 3-[({[(9H-fluoren-9-yl)methoxy]carbonyl}amino)methyl]benzoic acid (66.9 mg, 179 μmol) were solubilised in DMF (2.8 ml), 4-methylmorpholine (79 μl, 720 μmol, CAS-RN: 109-02-4) and COMU (153 mg, 358 μmol) were added and the mixture was stirred under argon for 2 h at rt. The mixture was diluted with brine and extracted with DCM. The organic phase was dried, evaporated and purified by preparative HPLC (C18, acetonitrile/water with 0.1% formic acid) to give 50.0 mg (95% purity, 25% yield) of the target compound.

LC-MS (Method 1): Rₜ=1.65 min; MS (ESIpos): m/z=1041 [M+H]⁺

¹H-NMR (400 MHz, DMSO-d6) δ[ppm]: 1.232 (0.49), 1.377 (16.00), 2.518 (1.84), 2.523 (1.15), 2.728 (0.80), 2.888 (1.02), 7.674 (0.44), 7.691 (0.54).

Intermediate 116

### tri-tert-butyl (3S,10S,14S)-1-[3-(aminomethyl)phenyl]-3-[(naphthalen-2-yl)methyl]-1,4,12-trioxo-2,5,11,13-tetraazahexadecane-10,14,16-tricarboxylate

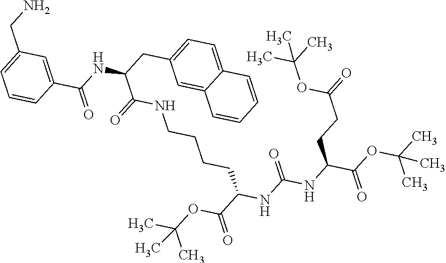 [SMILES: CC(C)(C)OC(=O)CC[C@H](NC(=O)N[C@@H](CCCCNC(=O)[C@H](Cc1ccc2ccccc2c1)NC(=O)c1cccc(CN)c1)C(=O)OC(C)(C)C)C(=O)OC(C)(C)C]

tri-tert-butyl (3S,10S,14S)-1-{3-[({[(9H-fluoren-9-yl)methoxy]carbonyl}amino)methyl]phenyl}-3-[(naphthalen-2-yl)methyl]-1,4,12-trioxo-2,5,11,13-tetraazahexadecane-10,14,16-tricarboxylate (49.0 mg, 47.1 μmol) was solubilised in DMF (1.1 ml), piperidine (47 μl, 470 μmol) was added and the mixture was stirred under argon for 2 h at rt. The mixture was evaporated and purified by preparative HPLC (C18, acetonitrile/water with 0.1% formic acid) to give 30.0 mg (85% purity, 66% yield) of the target compound.

LC-MS (Method 1): Rₜ=1.23 min; MS (ESIpos): m/z=819 [M+H]⁺

¹H-NMR (400 MHz, DMSO-d6) δ[ppm]: 1.233 (0.47), 1.380 (15.69), 1.383 (16.00), 2.332 (0.60), 2.518 (3.16), 2.523 (2.13), 2.728 (0.42), 2.888 (0.54), 7.438 (0.45), 7.457 (0.45), 7.790 (0.54), 7.797 (0.67), 7.810 (0.42).

### Example 27-A

### (3S,10S,14S)-1-[3-(aminomethyl)phenyl]-3-[(naphthalen-2-yl)methyl]-1,4,12-trioxo-2,5,11,13-tetraazahexadecane-10,14,16-tricarboxylic acid

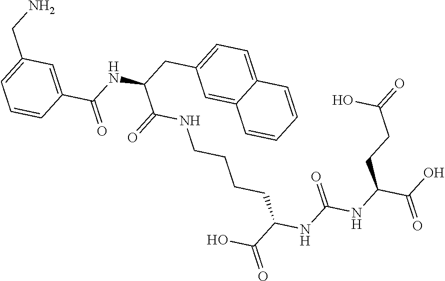 [SMILES: NCc1cccc(C(=O)N[C@@H](Cc2ccc3ccccc3c2)C(=O)NCCCC[C@H](NC(=O)N[C@@H](CCC(=O)O)C(=O)O)C(=O)O)c1]

tri-tert-butyl (3S,10S,14S)-1-[3-(aminomethyl)phenyl]-3-[(naphthalen-2-yl)methyl]-1,4,12-trioxo-2,5,11,13-tetraazahexadecane-10,14,16-tricarboxylate (10.0 mg, 85% purity, 10.4 μmol) was solubilised in DCM (330 μl), TFA (118 mg, 1.04 mmol) was added and the mixture was stirred under argon overnight at rt. The mixture was evaporated and purified by preparative HPLC (C18, acetonitrile/water with 0.1% formic acid) to give 6.00 mg (95% purity, 84% yield) of the target compound.

LC-MS (Method 1): Rₜ=0.75 min; MS (ESIpos): m/z=650 [M+H]⁺

¹H-NMR (400 MHz, DMSO-d6) δ[ppm]: 1.137 (0.94), 1.232 (1.30), 1.359 (0.94), 1.376 (0.86), 1.787 (1.01), 1.806 (1.08), 1.824 (0.50), 2.084 (1.37), 2.247 (0.94), 2.257 (1.08), 2.276 (0.50), 2.332 (3.03), 2.336 (1.30), 2.518 (16.00), 2.523 (10.45), 2.678 (1.30), 2.960 (0.43), 3.941 (0.58), 3.968 (0.65), 3.979 (0.58), 4.035 (3.24), 4.775 (0.65), 4.792 (0.65), 6.339 (0.65), 6.358 (0.65), 7.388 (0.72), 7.407 (1.95), 7.419 (1.01), 7.426 (1.59), 7.436 (2.09), 7.442 (1.08), 7.454 (1.01), 7.489 (1.23), 7.506 (1.51), 7.523 (1.08), 7.682 (1.01), 7.701 (0.86), 7.786 (1.80), 7.807 (2.31), 7.815 (1.73), 7.821 (2.74), 7.837 (1.01), 8.008 (0.72), 8.230 (1.37), 9.102 (0.65), 9.124 (0.58).

Intermediate 117

### tri-tert-butyl (3S,10S,14S)-3-[(naphthalen-2-yl)methyl]-1,4,12-trioxo-1-[3-({2-[4,7,10-tris(2-tert-butoxy-2-oxoethyl)-1,4,7,10-tetraazacyclododecan-1-yl]acetamido}methyl)phenyl]-2,5,11,13-tetraazahexadecane-10,14,16-tricarboxylate

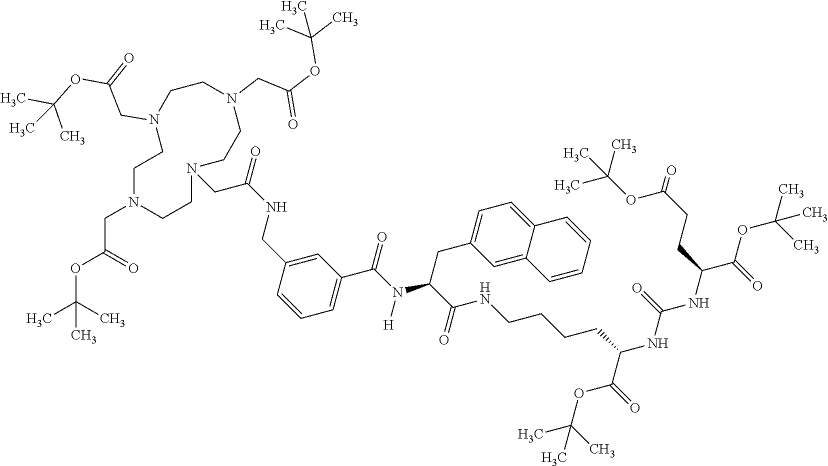 [SMILES: CC(C)(C)OC(=O)CC[C@H](NC(=O)N[C@@H](CCCCNC(=O)[C@H](Cc1ccc2ccccc2c1)NC(=O)c1cccc(CNC(=O)CN2CCN(CC(=O)OC(C)(C)C)CCN(CC(=O)OC(C)(C)C)CCN(CC(=O)OC(C)(C)C)CC2)c1)C(=O)OC(C)(C)C)C(=O)OC(C)(C)C]

N-[9,12-bis(2-tert-butoxy-2-oxoethyl)-2,2,6-trimethyl-4-oxo-3-oxa-6,9,12-triazatetradecan-14-yl]-N-ethylglycine (46.5 mg, 79.0 μmol), [(1H-benzotriazol-1-yl)oxy](dimethylamino)-N,N-dimethylmethaniminium hexafluoridophosphate(1−) (29.6 mg, 78.0 μmol; CAS-RN:94790-37-1) and N,N-diisopropylethylamine (10 μl, 59 μmol) were stirred in DMF (760 μl) for 10 min at rt. tri-tert-butyl (3S,10S,14S)-1-[3-(aminomethyl)phenyl]-3-[(naphthalen-2-yl)methyl]-1,4,12-trioxo-2,5,11,13-tetraazahexadecane-10,14,16-tricarboxylate (19.0 mg, 85% purity, 19.7 μmol) was added and the mixture was stirred overnight at rt. The mixture was evaporated and purified by preparative HPLC (C18, acetonitrile/water with 0.1% formic acid) to give 21.0 mg (80% purity, 62% yield) of the target compound.

LC-MS (Method 1): Rₜ=1.40 min; MS (ESIpos): m/z=1373 [M+H]⁺

¹H-NMR (400 MHz, DMSO-d6) δ[ppm]: 1.233 (0.50), 1.347 (3.02), 1.380 (14.40), 1.384 (16.00), 1.396 (4.35), 1.403 (2.48), 1.434 (1.48), 1.442 (1.18), 1.451 (1.04), 1.461 (3.39), 2.332 (0.60), 2.518 (3.38), 2.523 (2.16), 3.117 (0.44), 3.544 (0.43), 7.403 (0.48), 7.438 (0.52), 7.793 (0.62).

### Example 27-B

### (3S,10S,14S)-3-[(naphthalen-2-yl)methyl]-1,4,12-trioxo-1-[3-({2-[4,7,10-tris(carboxymethyl)-1,4,7,10-tetraazacyclododecan-1-yl]acetamido}methyl)phenyl]-2,5,11,13-tetraazahexadecane-10,14,16-tricarboxylic acid

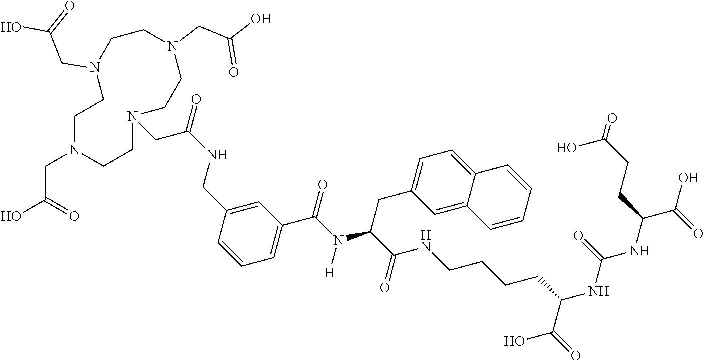 [SMILES: O=C(O)CC[C@H](NC(=O)N[C@@H](CCCCNC(=O)[C@H](Cc1ccc2ccccc2c1)NC(=O)c1cccc(CNC(=O)CN2CCN(CC(=O)O)CCN(CC(=O)O)CCN(CC(=O)O)CC2)c1)C(=O)O)C(=O)O]

tri-tert-butyl (3S,10S,14S)-3-[(naphthalen-2-yl)methyl]-1,4,12-trioxo-1-[3-({2-[4,7,10-tris(2-tert-butoxy-2-oxoethyl)-1,4,7,10-tetraazacyclododecan-1-yl]acetamido}methyl)phenyl]-2,5,11,13-tetraazahexadecane-10,14,16-tricarboxylate (18.0 mg, 80% purity, 10.5 μmol) was solubilised in DCM (330 μl), TFA (12.0 mg, 105 μmol) was added and the mixture was stirred under argon overnight at rt. The mixture was evaporated and purified by preparative HPLC (C18, acetonitrile/water with 0.1% formic acid) to give 7.50 mg (95% purity, 66% yield) of the target compound.

LC-MS (Method 1): Rₜ=0.74 min; MS (ESIpos): m/z=1037 [M+H]⁺

¹H-NMR (400 MHz, DMSO-d6) δ[ppm]: 1.137 (0.63), 1.232 (1.20), 1.279 (0.85), 1.297 (0.78), 1.353 (0.49), 1.373 (0.70), 1.392 (0.85), 1.621 (0.42), 1.713 (0.42), 1.907 (1.13), 2.084 (1.06), 2.207 (0.70), 2.229 (1.06), 2.331 (3.10), 2.336 (1.41), 2.518 (16.00), 2.523 (10.22), 2.620 (0.92), 2.673 (3.17), 2.678 (1.48), 3.018 (3.03), 3.110 (1.41), 3.258 (2.54), 3.438 (2.33), 4.009 (0.56), 4.023 (0.56), 4.042 (0.49), 4.061 (0.49), 4.077 (0.56), 4.279 (0.42), 4.698 (0.49), 4.718 (0.49), 6.366 (0.78), 6.385 (0.78), 7.285 (0.56), 7.398 (1.27), 7.415 (1.48), 7.430 (1.76), 7.435 (1.76), 7.453 (0.92), 7.500 (0.92), 7.521 (0.99), 7.562 (0.49), 7.579 (0.42), 7.785 (1.55), 7.806 (2.19), 7.824 (1.62), 7.851 (1.20), 7.990 (0.63), 8.137 (1.13), 8.228 (0.42), 8.751 (0.42).

### Example 27-B-²²⁷Th

### [²²⁷Th]Thorium (3S,10S,14S)-3-[(naphthalen-2-yl)methyl]-1,4,12-trioxo-1-{3-[(2-{4,7,10-tris[(carboxy-kappaO)methyl]-1,4,7,10-tetraazacyclododecan-1-yl-kappa⁴N¹,N⁴,N⁷,N¹⁰}acetamido)methyl]phenyl}-2,5,11,13-tetraazahexadecane-10,14,16-tricarboxylate

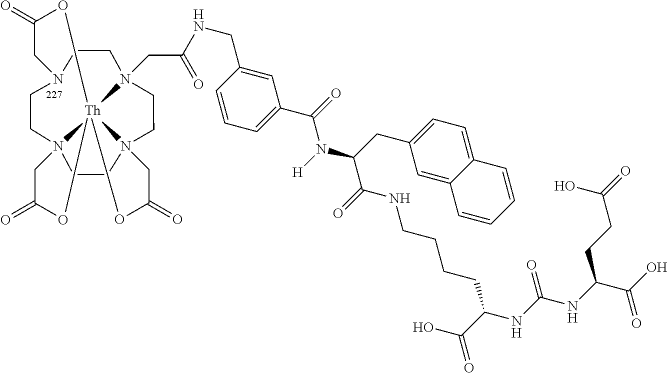

(3S,10S,14S)-3-[(naphthalen-2-yl)methyl]-1,4,12-trioxo-1-[3-({2-[4,7,10-tris(carboxymethyl)-1,4,7,10-tetraazacyclododecan-1-yl]acetamido}methyl)phenyl]-2,5,11,13-tetraazahexadecane-10,14,16-tricarboxylic acid (1.00 mg, 0.965 μmol) was dissolved in 400 mM sodium acetate buffer (pH 5) containing 0.5 mg/mL pABA. Thorium-227 in 400 mM sodium acetate buffer (pH 5) plus 20% EtOH was added giving a specific activity of 0.375 MBq/nmol and a RAC of 3.1 MBq/mL. The mixture was incubated at 95° C. for 60 min. The labelling efficiency was determined to be 95.7% by iTLC.

#28 Linker

Intermediate 118

### tri-tert-butyl (4S,11S,15S)-4-[(naphthalen-2-yl)methyl]-1-[3-(4-nitrophenyl)-1H-pyrazol-1-yl]-2,5,13-trioxo-3,6,12,14-tetraazaheptadecane-11,15,17-tricarboxylate

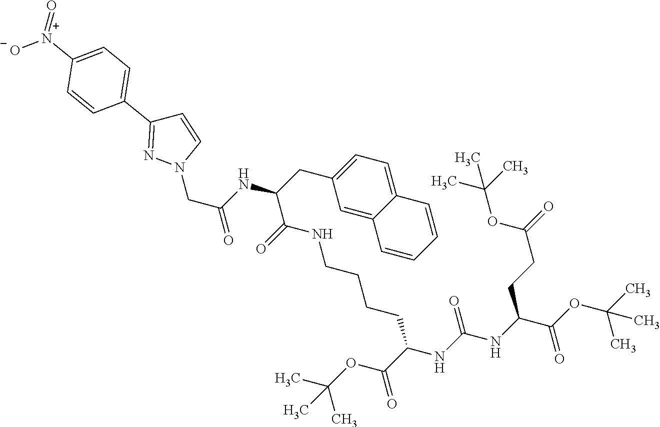 [SMILES: CC(C)(C)OC(=O)CC[C@H](NC(=O)N[C@@H](CCCCNC(=O)[C@H](Cc1ccc2ccccc2c1)NC(=O)Cn1ccc(-c2ccc([N+](=O)[O-])cc2)n1)C(=O)OC(C)(C)C)C(=O)OC(C)(C)C]

di-tert-butyl (2S)-2-({[(2S)-6-{[(2S)-2-amino-3-(naphthalen-2-yl)propanoyl]amino}-1-tert-butoxy-1-oxohexan-2-yl]carbamoyl}amino)pentanedioate (100 mg, 146 μmol) and [3-(4-nitrophenyl)-1H-pyrazol-1-yl]acetic acid (32.8 mg, 133 μmol) were solubilised in DMF (3.1 ml), 4-methylmorpholine (59 μl, 530 μmol, CAS-RN: 109-02-4) and HATU (75.7 mg, 199 μmol) were added and the mixture was stirred under argon at rt. The mixture was evaporated and purified by preparative HPLC (C18, acetonitrile/water with 0.1% formic acid) to give 57.0 mg (90% purity, 38% yield) of the target compound.

LC-MS (Method 1): Rₜ=1.53 min; MS (ESIpos): m/z=915 [M+H]⁺

¹H-NMR (400 MHz, DMSO-d6) δ[ppm]: 1.379 (16.00), 2.518 (1.05), 2.522 (0.66), 2.728 (0.40), 2.888 (0.52), 6.867 (0.63), 6.873 (0.55), 7.708 (0.60), 7.714 (0.55), 7.795 (0.46), 7.818 (0.44), 7.971 (0.70), 7.993 (0.84), 8.217 (0.92), 8.240 (0.72).

Intermediate 119

### tri-tert-butyl (4S,11S,15S)-1-[3-(4-aminophenyl)-1H-pyrazol-1-yl]-4-[(naphthalen-2-yl)methyl]-2,5,13-trioxo-3,6,12,14-tetraazaheptadecane-11,15,17-tricarboxylate

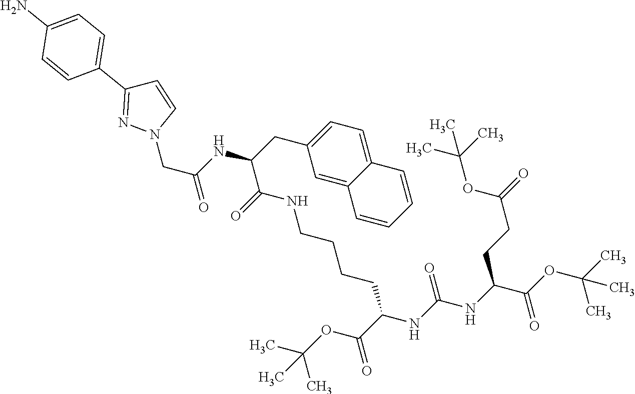 [SMILES: CC(C)(C)OC(=O)CC[C@H](NC(=O)N[C@@H](CCCCNC(=O)[C@H](Cc1ccc2ccccc2c1)NC(=O)Cn1ccc(-c2ccc(N)cc2)n1)C(=O)OC(C)(C)C)C(=O)OC(C)(C)C]

tri-tert-butyl (4S,11S,15S)-4-[(naphthalen-2-yl)methyl]-1-[3-(4-nitrophenyl)-1H-pyrazol-1-yl]-2,5,13-trioxo-3,6,12,14-tetraazaheptadecane-11,15,17-tricarboxylate (57.0 mg, 90% purity, 56.1 μmol) was solubilised in MeOH (1.1 ml), palladium on carbon (600 μg, 10% purity, 5.6 μmol) was added and the mixture was purged with hydrogen. The mixture was stirred at rt under hydrogen atmosphere. The mixture was filtered over Celite, washed with MeOH and evaporated to give 48.0 mg (85% purity, 82% yield) of the target compound.

LC-MS (Method 1): Rₜ=1.41 min; MS (ESIpos): m/z=885 [M+H]⁺

¹H-NMR (400 MHz, DMSO-d6) δ[ppm]: 1.353 (0.46), 1.377 (8.35), 1.380 (16.00), 1.383 (14.91), 2.331 (0.52), 2.518 (2.91), 2.523 (1.83), 2.673 (0.52), 5.142 (0.46), 6.404 (0.52), 6.410 (0.50), 6.536 (0.57), 6.558 (0.59), 7.381 (0.76), 7.402 (0.54), 7.455 (0.43), 7.508 (0.52), 7.514 (0.50).

### Example 28-A

### (4S,11S,15S)-1-[3-(4-aminophenyl)-1H-pyrazol-1-yl]-4-[(naphthalen-2-yl)methyl]-2,5,13-trioxo-3,6,12,14-tetraazaheptadecane-11,15,17-tricarboxylic acid

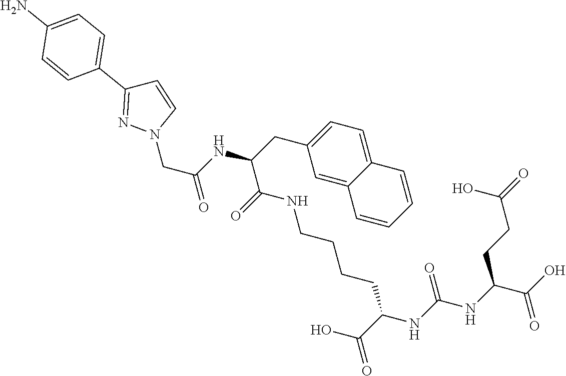 [SMILES: Nc1ccc(-c2ccn(CC(=O)N[C@@H](Cc3ccc4ccccc4c3)C(=O)NCCCC[C@H](NC(=O)N[C@@H](CCC(=O)O)C(=O)O)C(=O)O)n2)cc1]

tri-tert-butyl (4S,11S,15S)-1-[3-(4-aminophenyl)-1H-pyrazol-1-yl]-4-[(naphthalen-2-yl)methyl]-2,5,13-trioxo-3,6,12,14-tetraazaheptadecane-11,15,17-tricarboxylate (15.0 mg, 17.0 μmol) was solubilised in DCM (540 μl), TFA (65 μl, 850 μmol) was added and the mixture was stirred under argon overnight at rt. The mixture was evaporated and purified by preparative HPLC (C18, acetonitrile/water with 0.1% formic acid) to give 6.00 mg (80% purity, 40% yield) of the target compound.

LC-MS (Method 1): Rₜ=0.83 min; MS (ESIpos): m/z=716 [M+H]⁺

¹H-NMR (400 MHz, DMSO-d6) δ[ppm]: 1.192 (1.29), 1.211 (1.36), 1.231 (1.49), 1.270 (1.42), 1.289 (1.49), 1.413 (0.47), 1.431 (0.88), 1.446 (0.81), 1.466 (0.68), 1.550 (0.54), 1.563 (0.75), 1.583 (0.68), 1.676 (0.54), 1.690 (0.68), 1.711 (0.88), 1.725 (0.61), 1.886 (0.54), 1.907 (1.42), 1.921 (0.81), 2.208 (1.08), 2.226 (1.63), 2.245 (1.90), 2.266 (0.95), 2.336 (1.29), 2.518 (16.00), 2.522 (10.64), 2.539 (0.88), 2.673 (2.92), 2.678 (1.29), 2.924 (0.68), 2.938 (1.22), 2.958 (1.22), 2.971 (1.22), 2.992 (0.95), 3.022 (0.68), 3.038 (0.81), 3.055 (0.68), 3.071 (0.41), 3.096 (0.95), 3.109 (1.02), 3.129 (0.75), 3.143 (0.68), 3.982 (0.54), 4.003 (1.15), 4.015 (1.15), 4.036 (0.54), 4.066 (0.68), 4.079 (0.81), 4.087 (1.29), 4.100 (1.36), 4.107 (0.75), 4.121 (0.61), 4.549 (0.47), 4.569 (1.08), 4.583 (1.08), 4.603 (0.47), 4.703 (1.02), 4.743 (3.05), 4.766 (3.05), 4.806 (1.15), 6.259 (1.97), 6.280 (1.83), 6.304 (2.10), 6.324 (2.03), 6.408 (4.20), 6.414 (4.14), 6.547 (3.39), 6.569 (3.46), 6.585 (0.47), 6.591 (0.47), 7.361 (1.56), 7.365 (1.63), 7.386 (6.44), 7.407 (4.47), 7.431 (0.54), 7.444 (2.17), 7.446 (2.58), 7.457 (3.39), 7.467 (2.92), 7.470 (2.24), 7.482 (0.54), 7.511 (4.47), 7.517 (4.34), 7.591 (0.54), 7.596 (0.68), 7.601 (0.61), 7.658 (0.68), 7.672 (2.98), 7.777 (2.51), 7.790 (1.63), 7.799 (2.78), 7.814 (1.36), 7.829 (1.49), 7.840 (1.29), 7.853 (1.36), 8.090 (0.75), 8.105 (1.56), 8.119 (0.95), 8.349 (1.69), 8.369 (1.63).

Intermediate 120

### tri-tert-butyl (4S,11S,15S)-4-[(naphthalen-2-yl)methyl]-2,5,13-trioxo-1-[3-(4-{2-[4,7,10-tris(2-tert-butoxy-2-oxoethyl)-1,4,7,10-tetraazacyclododecan-1-yl]acetamido}phenyl)-1H-pyrazol-1-yl]-3,6,12,14-tetraazaheptadecane-11,15,17-tricarboxylate

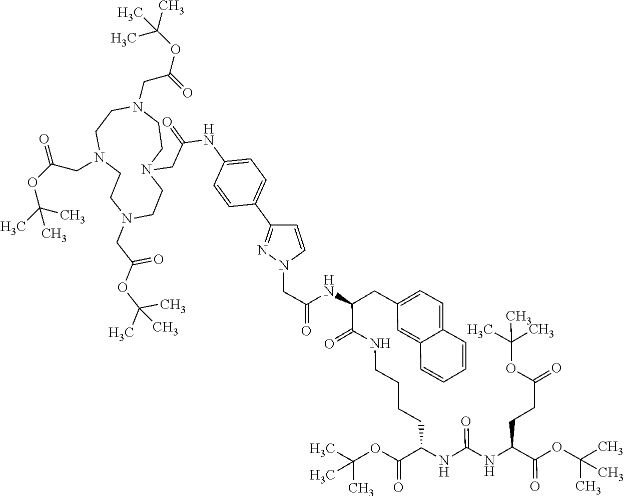 [SMILES: CC(C)(C)OC(=O)CC[C@H](NC(=O)N[C@@H](CCCCNC(=O)[C@H](Cc1ccc2ccccc2c1)NC(=O)Cn1ccc(-c2ccc(NC(=O)CN3CCN(CC(=O)OC(C)(C)C)CCN(CC(=O)OC(C)(C)C)CCN(CC(=O)OC(C)(C)C)CC3)cc2)n1)C(=O)OC(C)(C)C)C(=O)OC(C)(C)C]

N-[9,12-bis(2-tert-butoxy-2-oxoethyl)-2,2,6-trimethyl-4-oxo-3-oxa-6,9,12-triazatetradecan-14-yl]-N-ethylglycine (82.6 mg, 140 μmol), [(1H-benzotriazol-1-yl)oxy](dimethylamino)-N,N-dimethylmethaniminium hexafluoridophosphate(1−) (52.5 mg, 139 μmol; CAS-RN:94790-37-1) and N,N-diisopropylethylamine (18 μl, 110 μmol) were stirred in DMF (1.4 ml) for 10 min at rt. tri-tert-butyl (4S,11S,15S)-1-[3-(4-aminophenyl)-1H-pyrazol-1-yl]-4-[(naphthalen-2-yl)methyl]-2,5,13-trioxo-3,6,12,14-tetraazaheptadecane-11,15,17-tricarboxylate (31.0 mg, 35.1 μmol) was added and the mixture was stirred overnight at rt. The mixture was evaporated and purified by preparative HPLC (C18, acetonitrile/water with 0.1% formic acid) to give 40.0 mg (80% purity, 63% yield) of the target compound.

LC-MS (Method 1): Rₜ=1.35 min; MS (ESIpos): m/z=1439 [M+H]⁺

¹H-NMR (400 MHz, DMSO-d6) δ[ppm]: 1.354 (2.79), 1.380 (16.00), 1.411 (1.40), 1.432 (1.31), 1.439 (1.17), 1.451 (0.62), 1.467 (2.57), 2.331 (0.73), 2.518 (4.01), 2.523 (2.57), 2.673 (0.73), 7.606 (0.55).

### Example 28-B

### (4S,11S,15S)-4-[(naphthalen-2-yl)methyl]-2,5,13-trioxo-1-[3-(4-{2-[4,7,10-tris(carboxymethyl)-1,4,7,10-tetraazacyclododecan-1-yl]acetamido}phenyl)-1H-pyrazol-1-yl]-3,6,12,14-tetraazaheptadecane-11,15,17-tricarboxylic acid

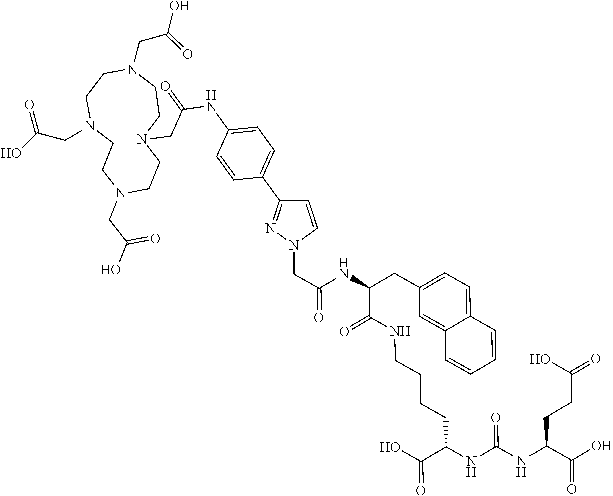 [SMILES: O=C(O)CC[C@H](NC(=O)N[C@@H](CCCCNC(=O)[C@H](Cc1ccc2ccccc2c1)NC(=O)Cn1ccc(-c2ccc(NC(=O)CN3CCN(CC(=O)O)CCN(CC(=O)O)CCN(CC(=O)O)CC3)cc2)n1)C(=O)O)C(=O)O]

tri-tert-butyl (4S,11S,15S)-4-[(naphthalen-2-yl)methyl]-2,5,13-trioxo-1-[3-(4-{2-[4,7,10-tris(2-tert-butoxy-2-oxoethyl)-1,4,7,10-tetraazacyclododecan-1-yl]acetamido}phenyl)-1H-pyrazol-1-yl]-3,6,12,14-tetraazaheptadecane-11,15,17-tricarboxylate (39.0 mg, 90% purity, 24.4 μmol) was solubilised in DCM (1.6 ml), TFA (190 μl, 2.4 mmol) was added and the mixture was stirred under argon overnight at rt. The mixture was evaporated and purified by preparative HPLC (C18, acetonitrile/water with 0.1% formic acid) to give 10.0 mg (90% purity, 33% yield) of the target compound.

LC-MS (Method 1): Rₜ=0.73 min; MS (ESIpos): m/z=1102 [M+H]⁺

¹H-NMR (400 MHz, DMSO-d6) δ[ppm]: 1.137 (0.81), 1.190 (1.22), 1.210 (1.22), 1.232 (1.69), 1.268 (1.42), 1.287 (1.42), 1.427 (0.54), 1.441 (0.68), 1.462 (0.54), 1.547 (0.54), 1.560 (0.68), 1.580 (0.54), 1.677 (0.47), 1.691 (0.68), 1.712 (0.74), 1.725 (0.61), 1.907 (1.28), 1.919 (0.68), 2.207 (0.95), 2.225 (1.42), 2.244 (1.62), 2.266 (0.81), 2.336 (1.35), 2.518 (16.00), 2.522 (10.53), 2.539 (0.68), 2.673 (2.97), 2.678 (1.35), 2.727 (0.61), 2.888 (1.69), 2.978 (4.66), 2.999 (5.00), 3.016 (4.73), 3.107 (1.22), 3.121 (1.28), 3.141 (1.08), 3.155 (1.01), 3.559 (2.90), 3.583 (2.77), 3.979 (0.47), 3.999 (0.95), 4.012 (1.01), 4.032 (0.47), 4.065 (0.61), 4.078 (0.68), 4.086 (1.15), 4.098 (1.15), 4.106 (0.61), 4.119 (0.54), 4.552 (0.47), 4.572 (0.95), 4.586 (1.08), 4.606 (0.47), 4.751 (0.88), 4.792 (1.76), 4.829 (1.82), 4.870 (0.95), 6.271 (1.35), 6.291 (1.28), 6.316 (1.42), 6.337 (1.35), 6.584 (3.11), 6.590 (2.97), 7.376 (1.49), 7.397 (1.62), 7.437 (0.47), 7.449 (1.69), 7.452 (2.57), 7.463 (2.97), 7.471 (2.70), 7.476 (1.96), 7.488 (0.54), 7.597 (3.04), 7.602 (2.90), 7.642 (1.22), 7.663 (4.93), 7.684 (3.85), 7.791 (2.63), 7.811 (3.17), 7.821 (1.35), 7.837 (1.42), 7.845 (1.15), 7.860 (1.28), 8.097 (1.08), 8.133 (1.62), 8.443 (1.22), 8.463 (1.22), 10.209 (1.42).

### Example 28-B-²²⁷Th

### [²²⁷Th]Thorium (4S,11S,15S)-4-[(naphthalen-2-yl)methyl]-2,5,13-trioxo-1-{3-[4-(2-{4,7,10-tris[(carboxy-kappaO)methyl]-1,4,7,10-tetraazacyclododecan-1-yl-kappa⁴N¹,N⁴,N⁷,N¹⁰}acetamido)phenyl]-1H-pyrazol-1-yl}-3,6,12,14-tetraazaheptadecane-11,15,17-tricarboxylate

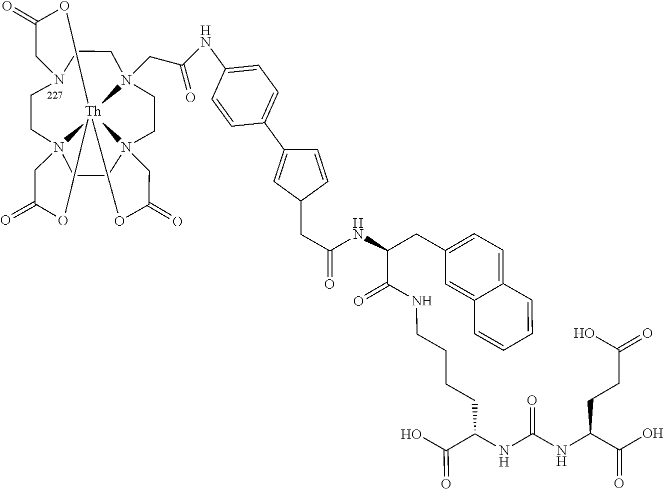

(4S,11S,15S)-4-[(naphthalen-2-yl)methyl]-2,5,13-trioxo-1-[3-(4-{2-[4,7,10-tris(carboxymethyl)-1,4,7,10-tetraazacyclododecan-1-yl]acetamido}phenyl)-1H-pyrazol-1-yl]-3,6,12,14-tetraazaheptadecane-11,15,17-tricarboxylic acid was dissolved in 400 mM sodium acetate buffer (pH 5) containing 0.5 mg/mL pABA. Thorium-227 in 400 mM sodium acetate buffer (pH 5) plus 20% EtOH was added giving a specific activity of 0.375 MBq/nmol and a RAC of 3.1 MBq/mL. The mixture was incubated at 95° C. for 60 min. The labelling efficiency was determined to be 79.2% by iTLC.

#29 Linker

Intermediate 121

### tri-tert-butyl (3S,10S,14S)-3-[(naphthalen-2-yl)methyl]-1-(4-nitrophenoxy)-1,4,12-trioxo-2,5,11,13-tetraazahexadecane-10,14,16-tricarboxylate

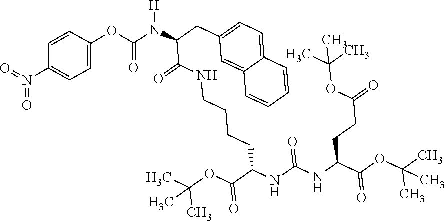 [SMILES: CC(C)(C)OC(=O)CC[C@H](NC(=O)N[C@@H](CCCCNC(=O)[C@H](Cc1ccc2ccccc2c1)NC(=O)Oc1ccc([N+](=O)[O-])cc1)C(=O)OC(C)(C)C)C(=O)OC(C)(C)C]

di-tert-butyl (2S)-2-({[(2S)-6-{[(2S)-2-amino-3-(naphthalen-2-yl)propanoyl]amino}-1-tert-butoxy-1-oxohexan-2-yl]carbamoyl}amino)pentanedioate (40.0 mg, 80% purity, 46.7 μmol) was solubilised in THF (1.9 ml), 4-nitrophenyl carbonochloridate (11.3 mg, 56.1 μmol) was added and the mixture was stirred for 3 h at 60° C. The mixture was evaporated to give 39.7 mg (99% yield) of the target compound.

LC-MS (Method 1): Rt=1.55 min; MS (ESIpos): m/z=851 [M+H]⁺

Intermediate 122

### tri-tert-butyl (5S,12S,16S)-1-[(1r,4S)-4-{[(tert-butoxycarbonyl)amino]methyl}cyclohexyl]-5-[(naphthalen-2-yl)methyl]-3,6,14-trioxo-2,4,7,13,15-pentaazaoctadecane-12,16,18-tricarboxylate

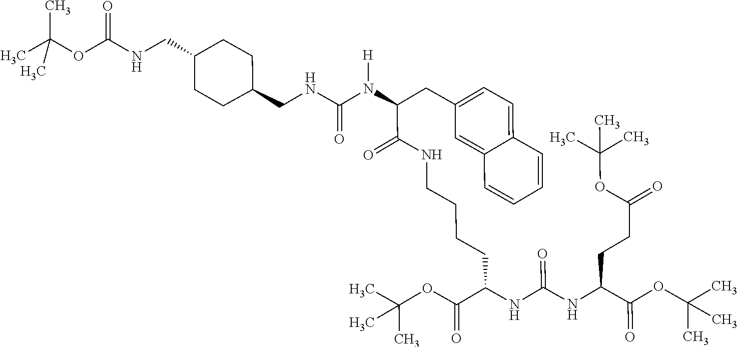 [SMILES: CC(C)(C)OC(=O)CC[C@H](NC(=O)N[C@@H](CCCCNC(=O)[C@H](Cc1ccc2ccccc2c1)NC(=O)NC[C@H]1CC[C@H](CNC(=O)OC(C)(C)C)CC1)C(=O)OC(C)(C)C)C(=O)OC(C)(C)C]

tert-butyl {[(1r,4r)-4-(aminomethyl)cyclohexyl]methyl}carbamate (13.6 mg, 56.1 μmol) was solubilised in DCM (1.5 ml), N,N-diisopropylethylamine (41 μl, 230 μmol) and tri-tert-butyl (3S,10S,14S)-3-[(naphthalen-2-yl)methyl]-1-(4-nitrophenoxy)-1,4,12-trioxo-2,5,11,13-tetraazahexadecane-10,14,16-tricarboxylate (39.7 mg, 46.7 μmol) were added and the mixture was stirred for 3 h at rt. The mixture was evaporated and purified by preparative HPLC (C18, acetonitrile/water with 0.1% formic acid) to give 30.0 mg (85% purity, 57% yield) of the target compound.

LC-MS (Method 1): Rₜ=1.57 min; MS (ESIpos): m/z=954 [M+H]⁺

¹H-NMR (400 MHz, DMSO-d6) δ[ppm]: 0.743 (0.41), 1.352 (3.00), 1.366 (7.77), 1.380 (16.00), 2.181 (0.43), 2.518 (2.02), 2.523 (1.22), 2.728 (0.43), 5.758 (10.86), 7.622 (0.41), 7.775 (0.55), 7.796 (0.51).

### Example 29-A

### (5S,12S,16S)-1-[(1r,4S)-4-(aminomethyl)cyclohexyl]-5-[(naphthalen-2-yl)methyl]-3,6,14-trioxo-2,4,7,13,15-pentaazaoctadecane-12,16,18-tricarboxylic acid

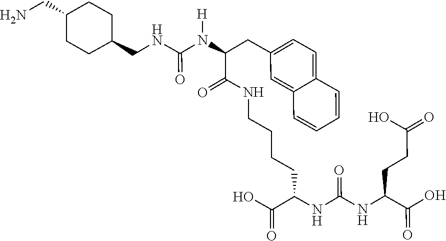 [SMILES: NC[C@H]1CC[C@H](CNC(=O)N[C@@H](Cc2ccc3ccccc3c2)C(=O)NCCCC[C@H](NC(=O)N[C@@H](CCC(=O)O)C(=O)O)C(=O)O)CC1]

tri-tert-butyl (5S,12S,16S)-1-[(1r,4S)-4-{[(tert-butoxycarbonyl)amino]methyl}cyclohexyl]-5-[(naphthalen-2-yl)methyl]-3,6,14-trioxo-2,4,7,13,15-pentaazaoctadecane-12,16,18-tricarboxylate (29.0 mg, 30.4 μmol) was solubilised in DCM (970 μl), TFA (230 μl, 3.0 mmol) was added and the mixture was stirred under argon overnight at rt. The mixture was evaporated and purified by preparative HPLC (C18, acetonitrile/water with 0.1% formic acid) to give 6.20 mg (90% purity, 27% yield) of the target compound.

LC-MS (Method 1): Rₜ=0.73 min; MS (ESIpos): m/z=685 [M+H]⁺

¹H-NMR (400 MHz, DMSO-d6) δ[ppm]: 0.763 (1.11), 0.794 (3.34), 0.820 (3.41), 0.851 (1.39), 1.137 (2.78), 1.175 (0.70), 1.232 (1.32), 1.286 (1.88), 1.300 (2.02), 1.313 (2.09), 1.415 (1.32), 1.430 (1.46), 1.449 (1.11), 1.616 (1.95), 1.663 (1.11), 1.699 (2.43), 1.726 (2.02), 1.762 (1.04), 1.779 (1.11), 1.795 (0.77), 1.814 (0.56), 1.906 (0.77), 2.084 (12.52), 2.115 (1.18), 2.162 (0.42), 2.181 (0.56), 2.199 (1.18), 2.217 (1.11), 2.232 (0.83), 2.240 (0.97), 2.261 (1.74), 2.282 (0.90), 2.298 (0.90), 2.318 (1.60), 2.322 (3.13), 2.327 (4.03), 2.331 (2.99), 2.336 (1.39), 2.518 (16.00), 2.523 (10.43), 2.590 (3.69), 2.607 (3.55), 2.636 (0.77), 2.659 (1.95), 2.664 (3.48), 2.669 (5.01), 2.673 (3.62), 2.678 (1.74), 2.854 (1.25), 2.870 (2.50), 2.888 (1.95), 2.904 (2.16), 2.926 (1.32), 3.034 (1.25), 3.047 (1.46), 3.067 (1.18), 3.081 (1.04), 3.131 (0.97), 3.147 (1.32), 3.164 (1.25), 3.181 (1.11), 3.932 (1.11), 3.947 (1.53), 3.966 (1.53), 3.978 (1.18), 4.334 (0.63), 4.356 (1.25), 4.370 (1.32), 4.393 (0.63), 6.176 (0.77), 6.306 (1.18), 6.327 (1.11), 6.539 (0.97), 6.729 (0.70), 7.341 (2.02), 7.344 (2.09), 7.362 (2.23), 7.365 (2.16), 7.418 (0.70), 7.422 (0.83), 7.435 (2.16), 7.439 (1.95), 7.448 (2.16), 7.453 (3.69), 7.459 (2.37), 7.467 (1.81), 7.471 (2.09), 7.485 (0.83), 7.489 (0.63), 7.653 (3.83), 7.781 (6.54), 7.801 (5.63), 7.838 (1.95), 7.843 (2.02), 7.860 (1.81), 8.195 (1.60).

#30 Linker

Intermediate 123

### tri-tert-butyl (5S,12S,16S)-1-[1-(tert-butoxycarbonyl)piperidin-4-yl]-5-[(naphthalen-2-yl)methyl]-3,6,14-trioxo-2,4,7,13,15-pentaazaoctadecane-12,16,18-tricarboxylate

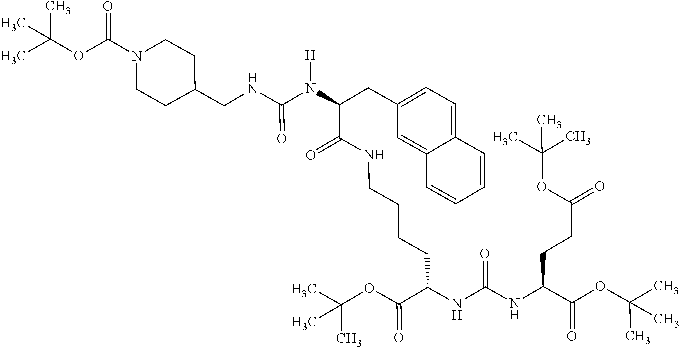 [SMILES: CC(C)(C)OC(=O)CC[C@H](NC(=O)N[C@@H](CCCCNC(=O)[C@H](Cc1ccc2ccccc2c1)NC(=O)NCC1CCN(C(=O)OC(C)(C)C)CC1)C(=O)OC(C)(C)C)C(=O)OC(C)(C)C]

tert-butyl 4-(aminomethyl)piperidine-1-carboxylate (12 μl, 56 μmol) was solubilised in DCM (1.5 ml), N,N-diisopropylethylamine (41 μl, 230 μmol) and tri-tert-butyl (3S,10S,14S)-3-[(naphthalen-2-yl)methyl]-1-(4-nitrophenoxy)-1,4,12-trioxo-2,5,11,13-tetraazahexadecane-10,14,16-tricarboxylate (39.7 mg, 46.7 μmol) were added and the mixture was stirred for 3 h at rt. The mixture was evaporated and purified by preparative HPLC (C18, acetonitrile/water with 0.1% formic acid) to give 40.0 mg (80% purity, 74% yield) of the target compound.

LC-MS (Method 1): Rₜ=1.56 min; MS (ESIpos): m/z=926 [M+H]⁺

¹H-NMR (400 MHz, DMSO-d6) δ[ppm]: 1.350 (1.71), 1.375 (10.29), 1.379 (16.00), 2.327 (0.69), 2.331 (0.46), 2.518 (2.63), 2.523 (1.71), 2.669 (0.69), 2.673 (0.46), 3.321 (0.46), 7.450 (0.46), 7.780 (0.46), 7.800 (0.46).

### Example 30-A

### (5S,12S,16S)-5-[(naphthalen-2-yl)methyl]-3,6,14-trioxo-1-(piperidin-4-yl)-2,4,7,13,15-pentaazaoctadecane-12,16,18-tricarboxylic acid

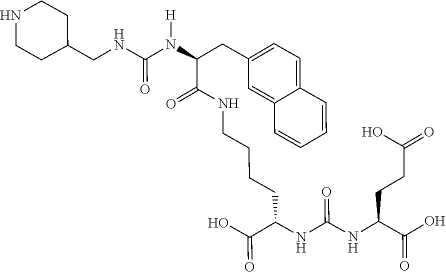 [SMILES: O=C(O)CC[C@H](NC(=O)N[C@@H](CCCCNC(=O)[C@H](Cc1ccc2ccccc2c1)NC(=O)NCC1CCNCC1)C(=O)O)C(=O)O]

tri-tert-butyl (5S,12S,16S)-1-[1-(tert-butoxycarbonyl)piperidin-4-yl]-5-[(naphthalen-2-yl)methyl]-3,6,14-trioxo-2,4,7,13,15-pentaazaoctadecane-12,16,18-tricarboxylate (39.0 mg, 80% purity, 33.7 μmol) was solubilised in DCM (1.1 ml), TFA (260 μl, 3.4 mmol) was added and the mixture was stirred under argon overnight at rt. The mixture was evaporated and purified by preparative HPLC (C18, acetonitrile/water with 0.1% formic acid) to give 3.20 mg (90% purity, 13% yield) of the target compound.

LC-MS (Method 1): Rₜ=0.69 min; MS (ESIpos): m/z=657 [M+H]⁺

¹H-NMR (400 MHz, DMSO-d6) δ[ppm]: 1.137 (4.47), 1.204 (1.05), 1.232 (2.45), 1.270 (2.72), 1.302 (4.26), 1.319 (4.33), 1.433 (1.19), 1.449 (1.40), 1.467 (1.47), 1.488 (1.26), 1.531 (2.03), 1.573 (2.31), 1.611 (1.33), 1.657 (1.12), 1.676 (1.12), 1.741 (1.40), 1.760 (3.63), 1.779 (3.84), 1.797 (1.61), 1.905 (0.98), 2.084 (0.70), 2.115 (1.96), 2.188 (0.49), 2.205 (1.05), 2.226 (1.82), 2.243 (3.07), 2.252 (1.82), 2.261 (1.47), 2.273 (2.86), 2.293 (1.47), 2.311 (1.12), 2.318 (1.54), 2.322 (3.07), 2.326 (4.33), 2.332 (3.35), 2.336 (1.47), 2.518 (16.00), 2.522 (10.62), 2.539 (0.98), 2.647 (0.91), 2.660 (2.31), 2.664 (4.26), 2.668 (6.01), 2.673 (4.96), 2.678 (3.21), 2.700 (2.10), 2.730 (1.96), 2.751 (1.26), 2.765 (1.61), 2.821 (0.98), 2.834 (1.19), 2.853 (1.33), 2.869 (2.17), 2.892 (1.82), 2.903 (2.59), 2.925 (2.86), 2.938 (1.54), 2.955 (1.19), 3.048 (1.96), 3.061 (2.31), 3.081 (1.82), 3.095 (1.68), 3.160 (3.14), 3.178 (3.35), 3.193 (3.00), 3.211 (2.52), 3.227 (2.38), 3.915 (1.89), 3.932 (1.89), 3.973 (0.91), 3.994 (1.82), 4.004 (1.82), 4.025 (0.91), 4.338 (1.05), 4.352 (1.26), 4.360 (2.03), 4.374 (2.03), 4.396 (0.98), 6.139 (1.33), 6.157 (1.33), 6.301 (2.03), 6.321 (1.96), 6.919 (1.33), 7.055 (1.05), 7.352 (3.14), 7.355 (3.21), 7.373 (3.35), 7.376 (3.42), 7.417 (0.91), 7.421 (1.19), 7.434 (3.21), 7.438 (3.07), 7.445 (3.28), 7.451 (6.64), 7.457 (3.56), 7.464 (3.07), 7.468 (3.21), 7.482 (1.19), 7.485 (0.91), 7.665 (5.94), 7.782 (9.08), 7.804 (9.71), 7.816 (1.75), 7.836 (2.93), 7.841 (3.00), 7.859 (2.86), 8.198 (2.24).

#31 Linker

Intermediate 124

### tri-tert-butyl (5S,12S,16S)-5-[(5-bromopyridin-2-yl)methyl]-1-(9H-fluoren-9-yl)-3,6,14-trioxo-2-oxa-4,7,13,15-tetraazaoctadecane-12,16,18-tricarboxylate

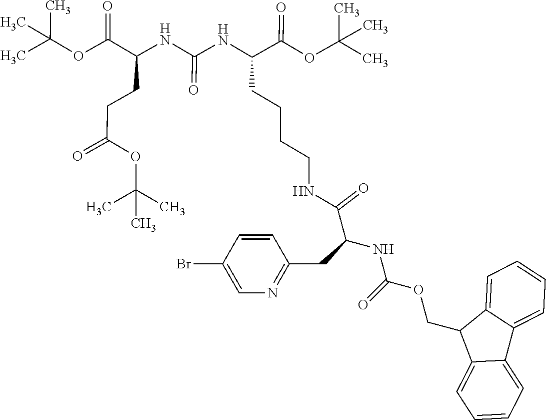 [SMILES: CC(C)(C)OC(=O)CC[C@H](NC(=O)N[C@@H](CCCCNC(=O)[C@H](Cc1ccc(Br)cn1)NC(=O)OCC1c2ccccc2-c2ccccc21)C(=O)OC(C)(C)C)C(=O)OC(C)(C)C]

di-tert-butyl N-{[(2S)-6-amino-1-tert-butoxy-1-oxohexan-2-yl]carbamoyl}-L-glutamate (300 mg, 95% purity, 584 μmol) and 3-(5-bromopyridin-2-yl)-N-{[(9H-fluoren-9-yl)methoxy]carbonyl}-L-alanine (410 mg, 877 μmol; CAS-RN:[282734-37-6]) were solubilised in DMF (4.5 ml), 4-methylmorpholine (190 μl, 1.8 mmol, CAS-RN: 109-02-4) and HATU (333 mg, 877 μmol) were added and the mixture was stirred under argon overnight at rt. The mixture was evaporated and purified by preparative HPLC (C18, acetonitrile/water with 0.1% formic acid) to give 254 mg (80% purity, 37% yield) of the target compound.

LC-MS (Method 1): Rₜ=1.62 min; MS (ESIpos): m/z=938 [M+H]⁺

Intermediate 125

### di-tert-butyl (2S)-2-({[(2S)-6-{[(2S)-2-amino-3-(5-bromopyridin-2-yl)propanoyl]amino}-1-tert-butoxy-1-oxohexan-2-yl]carbamoyl}amino)pentanedioate

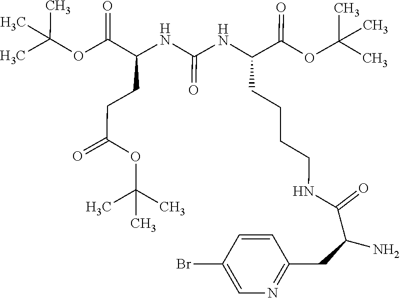 [SMILES: CC(C)(C)OC(=O)CC[C@H](NC(=O)N[C@@H](CCCCNC(=O)[C@@H](N)Cc1ccc(Br)cn1)C(=O)OC(C)(C)C)C(=O)OC(C)(C)C]

tri-tert-butyl (5S,12S,16S)-5-[(5-bromopyridin-2-yl)methyl]-1-(9H-fluoren-9-yl)-3,6,14-trioxo-2-oxa-4,7,13,15-tetraazaoctadecane-12,16,18-tricarboxylate (254 mg, 80% purity, 217 μmol) was solubilised in DMF (5.7 ml), piperidine (430 μl, 4.3 mmol) was added and the mixture was stirred under argon overnight at rt. The mixture was evaporated and purified by preparative HPLC (C18, acetonitrile/water with 0.1% formic acid) to give 38.0 mg (94% purity, 23% yield) of the target compound.

LC-MS (Method 1): Rₜ=1.09 min; MS (ESIpos): m/z=717 [M+H]⁺

Intermediate 126

### tri-tert-butyl (3S,10S,14S)-3-[(5-bromopyridin-2-yl)methyl]-1-{(1r,4S)-4-[({[(9H-fluoren-9-yl)methoxy]carbonyl}amino)methyl]cyclohexyl}-1,4,12-trioxo-2,5,11,13-tetraazahexadecane-10,14,16-tricarboxylate

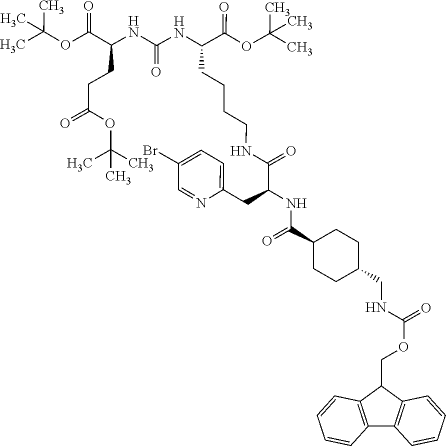 [SMILES: CC(C)(C)OC(=O)CC[C@H](NC(=O)N[C@@H](CCCCNC(=O)[C@H](Cc1ccc(Br)cn1)NC(=O)[C@H]1CC[C@H](CNC(=O)OCC2c3ccccc3-c3ccccc32)CC1)C(=O)OC(C)(C)C)C(=O)OC(C)(C)C]

di-tert-butyl (2S)-2-({[(2S)-6-{[(2S)-2-amino-3-(5-bromopyridin-2-yl)propanoyl]amino}-1-tert-butoxy-1-oxohexan-2-yl]carbamoyl}amino)pentanedioate (38.0 mg, 94% purity, 50.0 μmol) and (1r,4r)-4-[({[(9H-fluoren-9-yl)methoxy]carbonyl}amino)methyl]cyclohexane-1-carboxylic acid (28.4 mg, 75.0 μmol) were solubilised in DMF (380 μl), 4-methylmorpholine (16 μl, 150 μmol, CAS-RN: 109-02-4) and HATU (28.5 mg, 75.0 μmol) were added and the mixture was stirred under argon at rt. The mixture was evaporated and purified by preparative HPLC (C18, acetonitrile/water with 0.1% formic acid) to give 27.0 mg (95% purity, 48% yield) of the target compound.

LC-MS (Method 1): Rₜ=1.60 min; MS (ESIpos): m/z=1077 [M+H]⁺

Intermediate 127

### tri-tert-butyl (3S,10S,14S)-1-[(1r,4S)-4-(aminomethyl)cyclohexyl]-3-[(5-bromopyridin-2-yl)methyl]-1,4,12-trioxo-2,5,11,13-tetraazahexadecane-10,14,16-tricarboxylate

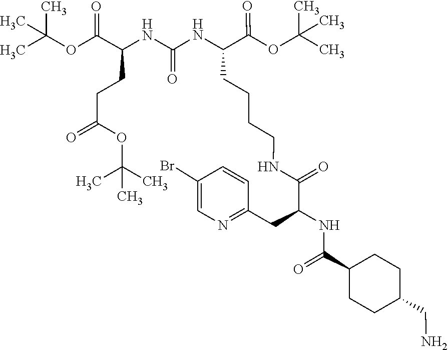 [SMILES: CC(C)(C)OC(=O)CC[C@H](NC(=O)N[C@@H](CCCCNC(=O)[C@H](Cc1ccc(Br)cn1)NC(=O)[C@H]1CC[C@H](CN)CC1)C(=O)OC(C)(C)C)C(=O)OC(C)(C)C]

tri-tert-butyl (3S,10S,14S)-3-[(5-bromopyridin-2-yl)methyl]-1-{(1r,4S)-4-[({[(9H-fluoren-9-yl)methoxy]carbonyl}amino)methyl]cyclohexyl}-1,4,12-trioxo-2,5,11,13-tetraazahexadecane-10,14,16-tricarboxylate (27.0 mg, 90% purity, 22.6 μmol) was solubilised in DMF (590 μl), piperidine (45 μl, 450 μmol) was added and the mixture was stirred under argon at rt. The mixture was evaporated and purified by preparative HPLC (C18, acetonitrile/water with 0.1% formic acid) to give 23.0 mg (27% purity, 32% yield) of the target compound.

LC-MS (Method 1): Rₜ=1.14 min; MS (ESIpos): m/z=856 [M+H]⁺

### Example 31-A

### N⁶—{N-[(1r,4S)-4-(aminomethyl)cyclohexane-1-carbonyl]-3-(5-bromopyridin-2-yl)-L-alanyl}-N²-{[(1S)-1,3-dicarboxypropyl]carbamoyl}-L-lysine

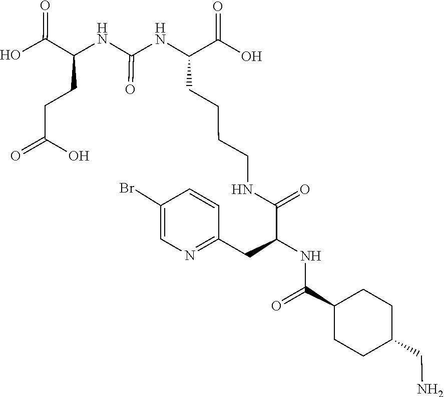 [SMILES: NC[C@H]1CC[C@H](C(=O)N[C@@H](Cc2ccc(Br)cn2)C(=O)NCCCC[C@H](NC(=O)N[C@@H](CCC(=O)O)C(=O)O)C(=O)O)CC1]

tri-tert-butyl (3S,10S,14S)-1-[(1r,4S)-4-(aminomethyl)cyclohexyl]-3-[(5-bromopyridin-2-yl)methyl]-1,4,12-trioxo-2,5,11,13-tetraazahexadecane-10,14,16-tricarboxylate (23.0 mg, 27% purity, 7.27 μmol) was solubilised in DCM (500 μl), TFA (500 μl, 6.5 mmol) was added and the mixture was stirred under argon overnight at rt. The mixture was evaporated and purified by preparative HPLC (C18, acetonitrile/water with 0.1% formic acid) to give 2.80 mg (85% purity, 48% yield) of the target compound.

LC-MS (Method 2): Rₜ=0.60 min; MS (ESIpos): m/z=687 [M+H]⁺

¹H-NMR (400 MHz, DMSO-d6) δ[ppm]: 0.850 (0.75), 0.913 (0.54), 1.107 (11.59), 1.165 (0.47), 1.232 (3.05), 1.333 (0.75), 1.352 (0.68), 1.437 (0.61), 1.574 (0.54), 1.670 (0.54), 2.056 (0.41), 2.331 (2.92), 2.336 (1.29), 2.518 (16.00), 2.523 (9.97), 2.539 (1.02), 2.593 (0.88), 2.612 (0.88), 2.669 (4.07), 2.673 (2.92), 2.678 (1.36), 2.889 (0.68), 2.915 (0.61), 2.926 (0.68), 2.948 (0.61), 3.073 (0.68), 3.093 (0.75), 3.108 (0.61), 3.804 (0.54), 3.934 (0.68), 6.400 (0.47), 6.421 (0.41), 7.222 (1.08), 7.242 (1.08), 7.798 (0.61), 7.918 (0.88), 7.924 (0.88), 7.938 (0.81), 7.945 (0.81), 8.089 (0.54), 8.112 (0.54), 8.571 (1.29), 8.576 (1.29).

#32 Linker

Intermediate 128

### methyl (2Z)-2-{[(benzyloxy)carbonyl]amino}-3-(quinolin-3-yl)prop-2-enoate

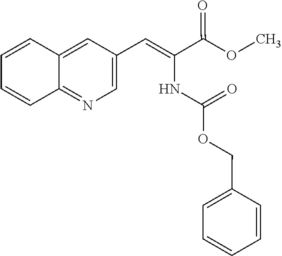 [SMILES: COC(=O)/C(=C/c1cnc2ccccc2c1)NC(=O)OCc1ccccc1]

1,8-Diazabicyclo(5.4.0)undec-7-ene (5.7 ml, 38 mmol) was added dropwise to a solution of methyl (benzyloxy)carbonyl]amino}(dimethoxyphosphoryl)acetate (12.6 g, 38.2 mmol) in DCM (120 ml). After stirring at rt for 10 min, a solution of quinoline-3-carbaldehyde (5.00 g, 31.8 mmol) in DCM (30 ml) was added. The reaction was stirred at rt for 2 h. The reaction solvent was removed under reduced pressure. The residue was diluted with EtOAc. The solution was washed with 1M HCl and brine. The organic phase was dried and filtered. The filtrate was concentrated under reduced pressure and purified by flash chromatography (SiO2, petroleum ether/EtOAc gradient 10%-50%) to give 5.62 g (96% purity, 47% yield) of the target compound.

¹H NMR (400 MHz, CDCl₃): δ [ppm]=9.03 (d, 1H), 8.21 (s, 1H), 8.08 (d, 1H), 7.78-7.66 (m, 2H), 7.59-7.51 (m, 1H), 7.50 (s, 1H), 7.39-7.23 (m, 5H), 6.75 (s, 1H), 5.10 (s, 2H), 3.89 (s, 3H).

Intermediate 129

### methyl N-[(benzyloxy)carbonyl]-3-quinolin-3-yl-D-alaninate

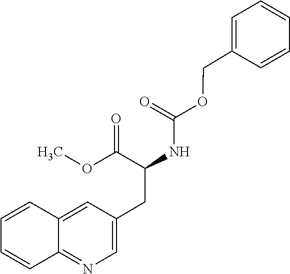 [SMILES: COC(=O)[C@H](Cc1cnc2ccccc2c1)NC(=O)OCc1ccccc1]

To a solution of methyl (2Z)-2-{[(benzyloxy)carbonyl]amino}-3-(quinolin-3-yl)prop-2-enoate (4.12 g, 96% purity, 10.9 mmol) in MeOH (200 ml) was added (R)—[Rh(COD)(MaxPHOS)]BF4 (388 mg, 655 μmol). The reaction mixture was stirred at rt for 40 h under hydrogen atmosphere (2.5 MPa). The reaction mixture was evaporated under reduced pressure. The residue was purified by flash chromatography (SiO2, petroleum ether/EtOAc gradient 20%-50%) to give 3.78 g (92% purity, 87% yield) of the target compound.

Intermediate 130

### N-[(benzyloxy)carbonyl]-3-quinolin-3-yl-D-alanine

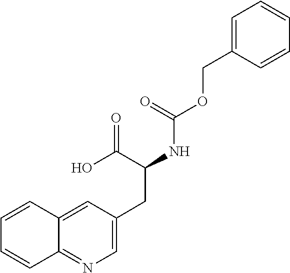 [SMILES: O=C(N[C@@H](Cc1cnc2ccccc2c1)C(=O)O)OCc1ccccc1]

To a solution of methyl N-[(benzyloxy)carbonyl]-3-quinolin-3-yl-D-alaninate (3.54 g, 9.71 mmol) in pyridine (70 ml) was added lithium iodide (15 ml, 29 mmol; CAS-RN:[10377-51-2]) at rt. The reaction mixture was refluxed for 16 h. The reaction solution was concentrated under reduced pressure and dissolved in water. The pH of the mixture was adjusted to 5 with HCl (1 M). The mixture was extracted with EtOAc. The organic phase was washed with brine, dried, filtered and concentrated under reduced pressure. The residue was purified by preparative HPLC (C18, acetonitrile/water with 0.1% formic acid) to give 1.54 g (98% purity, 44% yield) of the target compound.

LC-MS (Method D): Rₜ=0.599 min; MS (ESIpos): m/z=351.0 [M+H]⁺.

¹H NMR (400 MHz, DMSO-d₆) δ [ppm]=12.87 (s, 1H), 8.83 (d, 1H), 8.20 (d, 1H), 8.01 (d, 1H), 7.90 (d, 1H), 7.79 (d, 1H), 7.81-7.75 (m, 1H), 7.64-7.56 (m, 1H), 7.31-7.16 (m, 5H), 4.95 (s, 2H), 4.44-4.27 (m, 1H), 3.33-3.28 (m, 1H), 3.07 (dd, 1H).

Intermediate 131

### tri-tert-butyl (5R,12S,16S)-3,6,14-trioxo-1-phenyl-5-[(quinolin-3-yl)methyl]-2-oxa-4,7,13,15-tetraazaoctadecane-12,16,18-tricarboxylate

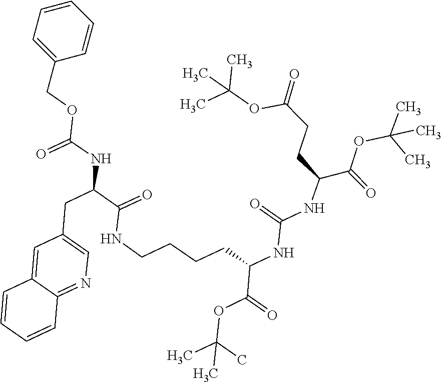

di-tert-butyl N-{[(2S)-6-amino-1-tert-butoxy-1-oxohexan-2-yl]carbamoyl}-L-glutamate (600 mg, 1.23 mmol) and N-[(benzyloxy)carbonyl]-3-quinolin-3-yl-D-alanine (431 mg, 1.23 mmol) were solubilised in DMF (9.5 ml), 4-methylmorpholine (340 μl, 3.1 mmol, CAS-RN: 109-02-4) and HATU (655 mg, 1.72 mmol) were added and the mixture was stirred under argon overnight at rt. The residue was diluted with DCM/propan-2-ol, washed with water and brine. The organic layer was dried and evaporated. The residue was purified by flash chromatography (SiO2, DCM/Ethanol gradient 0%-5%) to give 470 mg (99% purity, 46% yield) of the target compound.

LC-MS (Method 1): Rₜ=1.43 min; MS (ESIpos): m/z=821 [M+H]⁺

Intermediate 132

### di-tert-butyl (2S)-2-({[(2S)-6-{[(2R)-2-amino-3-(quinolin-3-yl)propanoyl]amino}-1-tert-butoxy-1-oxohexan-2-yl]carbamoyl}amino)pentanedioate

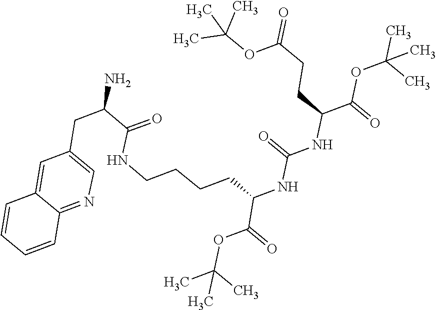 [SMILES: CC(C)(C)OC(=O)CC[C@H](NC(=O)N[C@@H](CCCCNC(=O)[C@H](N)Cc1cnc2ccccc2c1)C(=O)OC(C)(C)C)C(=O)OC(C)(C)C]

tri-tert-butyl (5R,12S,16S)-3,6,14-trioxo-1-phenyl-5-[(quinolin-3-yl)methyl]-2-oxa-4,7,13,15-tetraazaoctadecane-12,16,18-tricarboxylate (470 mg, 573 μmol) was solubilised in MeOH (4.6 ml), palladium on carbon (61.0 mg, 10% purity, 57.3 μmol) was added and the mixture was purged with hydrogen. The mixture was stirred for 2 h at rt under hydrogen atmosphere. The mixture was filtered over Celite, washed with MeOH and evaporated to give 380 mg (90% purity, 87% yield) of the target compound.

LC-MS (Method 1): Rt=1.13 min; MS (ESIpos): m/z=687 [M+H]⁺

Intermediate 133

### tri-tert-butyl (3R,10S,14S)-1-{5-[({[(9H-fluoren-9-yl)methoxy]carbonyl}amino)methyl]pyridin-2-yl}-1,4,12-trioxo-3-[(quinolin-3-yl)methyl]-2,5,11,13-tetraazahexadecane-10,14,16-tricarboxylate

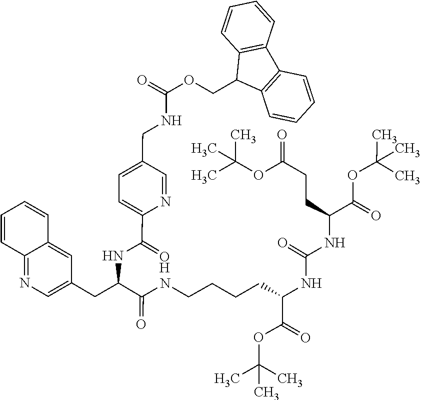 [SMILES: CC(C)(C)OC(=O)CC[C@H](NC(=O)N[C@@H](CCCCNC(=O)[C@@H](Cc1cnc2ccccc2c1)NC(=O)c1ccc(CNC(=O)OCC2c3ccccc3-c3ccccc32)cn1)C(=O)OC(C)(C)C)C(=O)OC(C)(C)C]

di-tert-butyl (2S)-2-({[(2S)-6-{[(2R)-2-amino-3-(quinolin-3-yl)propanoyl]amino}-1-tert-butoxy-1-oxohexan-2-yl]carbamoyl}amino)pentanedioate (330 mg, 481 μmol) and 5-[({[(9H-fluoren-9-yl)methoxy]carbonyl}amino)methyl]pyridine-2-carboxylic acid (164 mg, 437 μmol) were solubilised in DMF (2 ml), 4-methylmorpholine (140 μl, 1.3 mmol, CAS-RN: 109-02-4) and HATU (233 mg, 612 μmol) were added and the mixture was stirred under argon for 3 h at rt. The mixture was evaporated and purified by preparative HPLC (C18, acetonitrile/water with 0.1% formic acid) to give 340 mg (88% purity, 60% yield) of the target compound.

LC-MS (Method 1): Rt=1.53 min; MS (ESIpos): m/z=1043 [M+H]⁺

Intermediate 134

### tri-tert-butyl (3R,10S,14S)-1-[5-(aminomethyl)pyridin-2-yl]-1,4,12-trioxo-3-[(quinolin-3-yl)methyl]-2,5,11,13-tetraazahexadecane-10,14,16-tricarboxylate

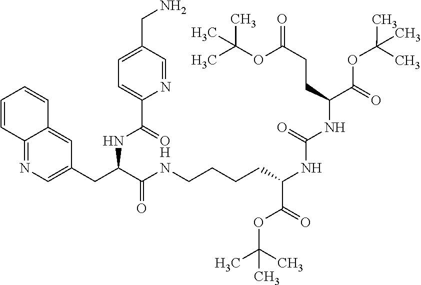

tri-tert-butyl (3R,10S,14S)-1-{5-[({[(9H-fluoren-9-yl)methoxy]carbonyl}amino)methyl]pyridin-2-yl}-1,4,12-trioxo-3-[(quinolin-3-yl)methyl]-2,5,11,13-tetraazahexadecane-10,14,16-tricarboxylate (340 mg, 326 μmol) was solubilised in DMF (5.0 ml), piperidine (650 μl, 6.5 mmol) was added and the mixture was stirred under argon overnight at rt. The mixture was evaporated and purified by preparative HPLC (C18, acetonitrile/water with 0.1% formic acid) to give 115 mg (99% purity, 43% yield) of the target compound.

LC-MS (Method 1): Rt=1.08 min; MS (ESIpos): m/z=821 [M+H]⁺

¹H-NMR (400 MHz, DMSO-d6) δ[ppm]: 1.362 (0.88), 1.373 (7.59), 1.379 (16.00), 2.518 (0.98), 2.522 (0.63), 3.873 (0.83), 7.907 (0.47), 8.728 (0.48), 8.734 (0.53).

### Example 32-A

### N⁶—{N-[5-(aminomethyl)pyridine-2-carbonyl]-3-(quinolin-3-yl)-L-alanyl}-N²-{[(1S)-1,3-dicarboxypropyl]carbamoyl}-L-lysine

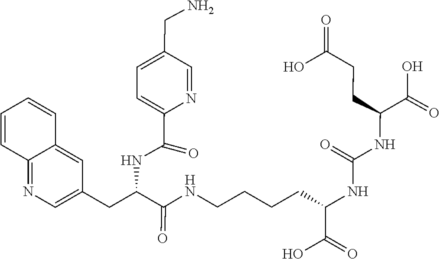 [SMILES: NCc1ccc(C(=O)N[C@@H](Cc2cnc3ccccc3c2)C(=O)NCCCC[C@H](NC(=O)N[C@@H](CCC(=O)O)C(=O)O)C(=O)O)nc1]

tri-tert-butyl (3S,10S,14S)-1-[5-(aminomethyl)pyridin-2-yl]-1,4,12-trioxo-3-[(quinolin-3-yl)methyl]-2,5,11,13-tetraazahexadecane-10,14,16-tricarboxylate (50.0 mg, 85% purity, 51.8 μmol) was solubilised in DCM (1.7 ml), TFA (800 μl, 10 mmol) was added and the mixture was stirred under argon overnight at rt. The mixture was evaporated and purified by preparative HPLC (C18, acetonitrile/water with 0.1% formic acid) to give 5.50 mg (99% purity, 16% yield) of the target compound.

LC-MS (Method 1): Rt=0.53 min; MS (ESIpos): m/z=653 [M+H]⁺

¹H-NMR (400 MHz, DMSO-d6) δ[ppm]: 1.232 (1.13), 1.288 (1.57), 1.326 (0.94), 1.342 (0.88), 1.392 (1.26), 1.410 (1.13), 1.422 (1.13), 1.443 (0.94), 1.459 (0.76), 1.605 (0.88), 1.658 (0.57), 1.671 (0.76), 1.691 (0.94), 1.706 (0.76), 1.727 (0.63), 1.747 (0.94), 1.764 (1.13), 1.781 (0.76), 1.798 (0.57), 1.906 (0.44), 2.074 (4.98), 2.168 (1.20), 2.186 (1.95), 2.206 (2.27), 2.227 (1.01), 2.244 (0.63), 2.331 (2.77), 2.336 (1.26), 2.518 (16.00), 2.522 (9.95), 2.673 (2.83), 2.678 (1.32), 2.686 (1.64), 3.070 (2.46), 3.084 (2.46), 3.226 (1.89), 3.247 (2.27), 3.259 (3.21), 3.281 (3.65), 3.301 (3.91), 3.312 (4.35), 3.333 (3.72), 3.345 (3.46), 3.944 (1.32), 3.959 (1.76), 3.978 (1.76), 3.990 (1.45), 4.011 (0.94), 4.032 (4.60), 4.799 (0.69), 4.813 (0.82), 4.821 (1.26), 4.835 (1.32), 4.856 (0.63), 6.124 (0.94), 6.140 (0.94), 6.411 (1.76), 6.431 (1.70), 7.523 (1.13), 7.526 (1.13), 7.543 (2.46), 7.560 (1.57), 7.563 (1.57), 7.656 (1.39), 7.659 (1.45), 7.673 (1.26), 7.677 (2.46), 7.681 (1.76), 7.694 (1.32), 7.698 (1.32), 7.835 (2.27), 7.854 (1.95), 7.909 (1.89), 7.927 (4.91), 7.945 (2.65), 7.952 (2.90), 7.957 (2.39), 7.973 (1.07), 7.978 (1.07), 8.111 (3.02), 8.115 (3.09), 8.172 (1.95), 8.205 (0.88), 8.219 (1.76), 8.232 (0.88), 8.656 (3.09), 8.731 (4.28), 8.736 (4.16), 8.903 (2.14), 8.925 (2.02).

Intermediate 135

### tri-tert-butyl (3R,10S,14S)-1,4,12-trioxo-3-[(quinolin-3-yl)methyl]-1-[5-({2-[4,7,10-tris(2-tert-butoxy-2-oxoethyl)-1,4,7,10-tetraazacyclododecan-1-yl]acetamido}methyl)pyridin-2-yl]-2,5,11,13-tetraazahexadecane-10,14,16-tricarboxylate

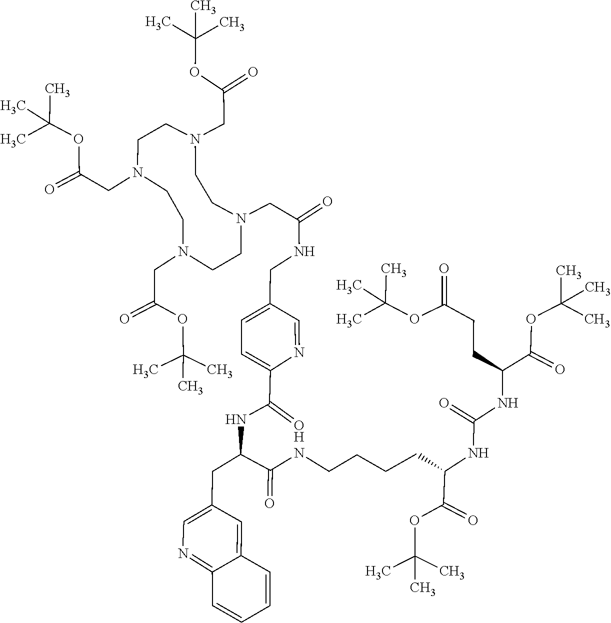

[4,7,10-tris(2-tert-butoxy-2-oxoethyl)-1,4,7,10-tetraazacyclododecan-1-yl]acetic acid (249 mg, 435 μmol; CAS-RN:[137076-54-1]), [(1H-benzotriazol-1-yl)oxy](dimethylamino)-N,N-dimethylmethaniminium hexafluoridophosphate(1−) (163 mg, 430 μmol; CAS-RN:94790-37-1) and N,N-diisopropylethylamine (53 μl, 330 μmol) were stirred in DMF (4.2 ml) for 10 min at rt. tri-tert-butyl (3S,10S,14S)-1-[5-(aminomethyl)pyridin-2-yl]-1,4,12-trioxo-3-[(quinolin-3-yl)methyl]-2,5,11,13-tetraazahexadecane-10,14,16-tricarboxylate (105 mg, 85% purity, 109 μmol) was added and the mixture was stirred overnight at rt. The mixture was evaporated and purified by preparative HPLC (C18, acetonitrile/water with 0.1% formic acid) to give 45.0 mg (30% yield) of the target compound.

LC-MS (Method 1): Rt=1.33 min; MS (ESIpos): m/z=1375 [M+H]⁺

¹H-NMR (400 MHz, DMSO-d6) δ[ppm]: 1.232 (0.41), 1.322 (1.44), 1.374 (5.64), 1.379 (16.00), 1.392 (2.09), 1.435 (1.52), 2.518 (4.14), 2.523 (2.69), 2.673 (0.78), 2.687 (0.46), 2.888 (0.41).

### Example 32-B

### N⁶-{3-(quinolin-3-yl)-N-[5-({2-[4,7,10-tris(carboxymethyl)-1,4,7,10-tetraazacyclododecan-1-yl]acetamido}methyl)pyridine-2-carbonyl]-D-alanyl}-N²-{[(1S)-1,3-dicarboxypropyl]carbamoyl}-L-lysine

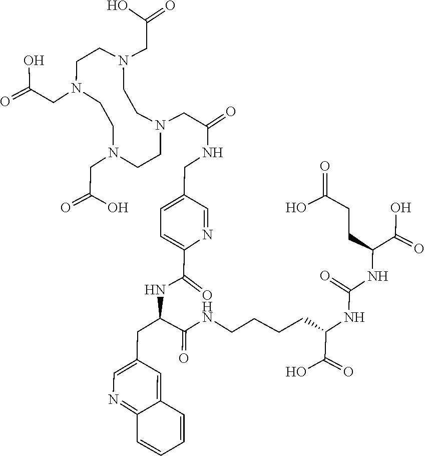 [SMILES: O=C(O)CC[C@H](NC(=O)N[C@@H](CCCCNC(=O)[C@@H](Cc1cnc2ccccc2c1)NC(=O)c1ccc(CNC(=O)CN2CCN(CC(=O)O)CCN(CC(=O)O)CCN(CC(=O)O)CC2)cn1)C(=O)O)C(=O)O]

tri-tert-butyl (3R,10S,14S)-1,4,12-trioxo-3-[(quinolin-3-yl)methyl]-1-[5-({2-[4,7,10-tris(2-tert-butoxy-2-oxoethyl)-1,4,7,10-tetraazacyclododecan-1-yl]acetamido}methyl)pyridin-2-yl]-2,5,11,13-tetraazahexadecane-10,14,16-tricarboxylate (20.0 mg, 14.5 μmol) was solubilised in DCM (470 μl), TFA (220 μl, 2.9 mmol) was added and the mixture was stirred under argon over the weekend at rt. The mixture was evaporated and purified by preparative HPLC (C18, acetonitrile/water with 0.1% formic acid) to give 1.50 mg (97% purity, 10% yield) of the target compound.

¹H-NMR (400 MHz, DMSO-d6) δ[ppm]: 1.232 (1.82), 1.316 (0.63), 1.334 (0.77), 1.352 (0.84), 1.455 (0.49), 1.470 (0.56), 1.590 (0.49), 1.694 (0.42), 1.715 (0.56), 1.731 (0.49), 1.871 (0.49), 2.084 (1.12), 2.205 (0.91), 2.226 (1.19), 2.233 (1.19), 2.254 (0.70), 2.327 (3.91), 2.331 (2.93), 2.336 (1.40), 2.518 (16.00), 2.523 (9.99), 2.589 (1.26), 2.669 (4.19), 2.673 (3.07), 2.678 (1.54), 2.915 (1.68), 3.037 (3.21), 3.103 (1.89), 3.995 (0.77), 4.008 (0.84), 4.028 (0.49), 4.050 (0.49), 4.070 (0.77), 4.084 (0.77), 4.104 (0.42), 4.389 (1.19), 4.805 (0.63), 4.821 (0.63), 6.300 (1.33), 6.320 (1.33), 7.529 (0.70), 7.547 (1.40), 7.564 (0.84), 7.661 (0.84), 7.664 (0.84), 7.682 (1.40), 7.699 (0.70), 7.845 (2.03), 7.863 (1.47), 7.938 (2.03), 7.959 (1.68), 8.111 (1.82), 8.243 (0.56), 8.629 (0.77), 8.743 (2.31), 8.748 (2.24), 8.771 (1.12), 8.791 (1.26).

### Example 32-B 227Th

### {N⁶-[3-(quinolin-3-yl)-N-{5-[(2-{4,7,10-tris[(carboxy-kappaO)methyl]-1,4,7,10-tetraazacyclododecan-1-yl-kappa²N¹,N⁴}acetamido)methyl]pyridine-2-carbonyl}-D-alanyl]-N²-{[(1S)-1,3-dicarboxypropyl]carbamoyl}-L-lysinato(3−)}(²²⁷Th)Thorium

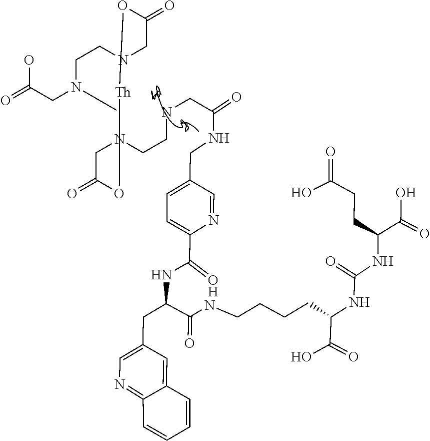

N⁶-{3-(quinolin-3-yl)-N-[5-({2-[4,7,10-tris(carboxymethyl)-1,4,7,10-tetraazacyclododecan-1-yl]acetamido}methyl)pyridine-2-carbonyl]-D-alanyl}-N2-{[(1S)-1,3-dicarboxypropyl]carbamoyl}-L-lysine was dissolved as 10 mM solution in DMSO and diluted to a 250 μM solution in 400 mM sodium acetate buffer (pH 5) containing 0.5 mg/mL pABA. 1.07 μl of this solution was used in a 40 μl labeling reaction. Thorium-227 in 400 mM sodium acetate buffer (pH 5) plus 20% EtOH was added giving a specific activity of 0.375 MBq/nmol and a RAC of 2.5 MBq/mL. The mixture was incubated at 95° C. for 60 min. The labelling efficiency was determined to be 98.2% by iTLC.

#33 Linker

Intermediate 136

### methyl 1-methoxynaphthalene-2-carboxylate

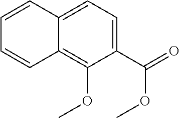 [SMILES: COC(=O)c1ccc2ccccc2c1OC]

To a solution of 1-hydroxynaphthalene-2-carboxylic acid (10.0 g, 53.1 mmol) and potassium carbonate (22.0 g, 159 mmol) in acetone (200 ml) was added iodomethane (13 ml, 210 mmol) at room temperature. The reaction mixture was refluxed for 16 hours. The mixture was filtered through a pad of celite and the filtrate was concentrated to give a residue. The residue was purified by column chromatography (1000 mesh, petroleum ether:ethyl acetate=100:1 to 50:1) to give methyl 1-methoxy-2-naphthoate (5.00 g, 44% yield) as yellow oil.

LC-MS (Method C): Rₜ=0.930 min; MS (ESIpos): m/z=217.1 [M+H]⁺.

¹H NMR (400 MHz, DMSO-d₆): 5 [ppm]=8.19 (d, 1H), 7.99 (d, 1H), 7.78-7.50 (m, 2H), 7.69-7.62 (m, 2H), 3.97 (s, 3H), 3.90 (s, 3H).

Intermediate 137

### (1-methoxynaphthalen-2-yl)methanol

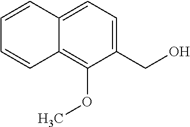

To a solution of methyl 1-methoxynaphthalene-2-carboxylate (14.0 g, 64.7 mmol) in toluene (200 ml) was added diisobutylaluminum hydride (97 ml, 1.0 M in toluene, 97 mmol; CAS-RN:[1191-15-7]) at −40° C. The mixture was stirred at 0° C. for 1 h. The mixture was quenched with saturated ammonium chloride and extracted with ethyl acetate. The organic phase was washed with brine, dried and concentrated under reduced pressure. The residue was purified by flash chromatography (SiO2, petroleum ether/EtOAc gradient 10%-50%) to give 12.0 g (98% yield) of the target compound.

Intermediate 138

### 1-methoxynaphthalene-2-carbaldehyde

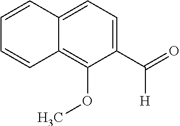

To a solution of morpholine (1.7 ml, 19 mmol) in tetrahydrofuran (40 ml) was added diisobutylaluminum hydride (1M in toluene) at 0° C. The mixture was stirred at 0° C. for 3 hours. A solution of methyl 1-methoxynaphthalene-2-carboxylate (2.00 g, 9.25 mmol) in tetrahydrofuran (5 ml) was added into the above mixture. The reaction mixture was stirred for 10 minutes. Diisobutylaluminum hydride (1M in toluene) was added. After stirring for 0.5 hour, the mixture was quenched with hydrochloric acid (1M) and extracted with ethyl acetate. The organic phase was washed with brine, dried over anhydrous sodium sulfate, filtered and concentrated to give a residue. The residue was purified by column chromatography (1000 mesh, petroleum ether:ethyl acetate=50:1 to 10:1) to give 1-methoxy-2-naphthaldehyde (600 mg, 35% yield) as yellow oil.

LC-MS (Method D): Rₜ=0.769 min; MS (ESIpos): m/z=187.1 [M+H]⁺.

¹H NMR (400 MHz, DMSO-d₆): 5 [ppm]=10.5 (s, 1H), 8.26 (d, 1H), 8.03 (d, 1H), 7.81-7.68 (m, 4H), 4.12 (s, 3H).

Intermediate 139

### methyl (2Z)-2-{[(benzyloxy)carbonyl]amino}-3-(1-methoxynaphthalen-2-yl)prop-2-enoate

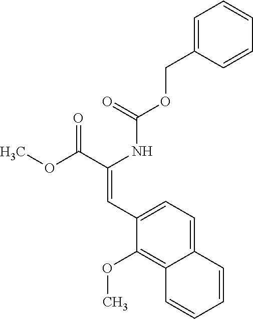 [SMILES: COC(=O)/C(=C/c1ccc2ccccc2c1OC)NC(=O)OCc1ccccc1]

To a solution of methyl {[(benzyloxy)carbonyl]amino}(dimethoxyphosphoryl)acetate (1.28 g, 3.87 mmol) in dichloromethane (10 ml) was added 1,8-diazabicyclo(5.4.0)undec-7-ene (0.580 ml, 3.9 mmol) at room temperature. After stirring for 0.5 hour, a solution of 1-methoxynaphthalene-2-carbaldehyde (600 mg, 3.22 mmol) in dichloromethane (5 ml) was added into above mixture. The reaction mixture was stirred at room temperature for 2 hours. The mixture was quenched with saturated ammonium chloride and extracted with ethyl acetate. The organic phase was washed with brine, dried over anhydrous sodium sulfate, filtered and concentrated to give a residue. The residue was purified by column chromatography (1000 mesh, petroleum ether:ethyl acetate=10:1 to 3:1) to give methyl (2Z)-2-{[(benzyloxy)carbonyl]amino}-3-(1-methoxy-2-naphthyl)acrylate (1.10 g, 87% yield) as brown oil.

LC-MS (Method D): Rₜ=0.899 min; MS (ESIpos): m/z=392.1 [M+H]⁺.

¹H NMR (400 MHz, DMSO-d₆): δ [ppm]=9.19 (s, 1H), 8.11-8.09 (m, 1H), 7.96-7.93 (m, 1H), 7.75-7.67 (m, 1H), 7.61-7.60 (m, 1H), 7.60-7.58 (m, 3H), 7.34 (br.s, 5H), 5.08 (s, 2H), 3.87 (s, 3H), 3.76 (s, 3H).

Intermediate 140

### methyl (2R)-2-{[(benzyloxy)carbonyl]amino}-3-(1-methoxynaphthalen-2-yl)propanoate

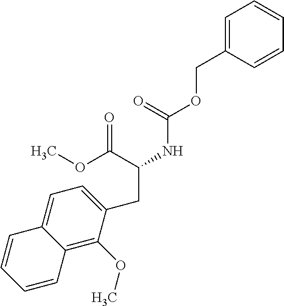 [SMILES: COC(=O)[C@@H](Cc1ccc2ccccc2c1OC)NC(=O)OCc1ccccc1]

To a solution of methyl (2Z)-2-{[(benzyloxy)carbonyl]amino}-3-(1-methoxynaphthalen-2-yl)prop-2-enoate (8.9 g, 22.7 mmol) in methanol (200 ml) were added (R)—[Rh(COD)(MaxPhos)]BF₄ (270 mg, 0.455 mmol). After stirring at room temperature for 16 hours under hydrogen atmosphere (15 psi), the mixture was concentrated to give a residue. The residue was purified by column chromatography (200-300 mesh, ethyl acetate) to give methyl (2S)-2-{[(benzyloxy)carbonyl]amino}-3-(1-methoxy-2-naphthyl) propanoate (8.5 g, 95% yield) as a yellow oil.

LC-MS (Method D): Rₜ=0.886 min; MS (ESIpos): m/z=350.1 [M+H]⁺.

Intermediate 141

### (2R)-2-{[(benzyloxy)carbonyl]amino}-3-(1-methoxynaphthalen-2-yl)propanoic acid

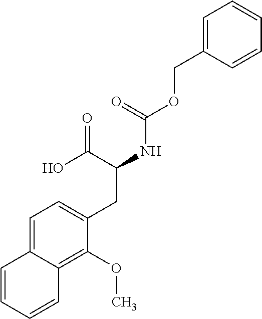 [SMILES: COc1c(C[C@H](NC(=O)OCc2ccccc2)C(=O)O)ccc2ccccc12]

To a solution of methyl (2R)-2-{[(benzyloxy)carbonyl]amino}-3-(1-methoxynaphthalen-2-yl)propanoate (8.50 g, 21.6 mmol) in tetrahydrofuran (100 ml) was added lithium hydroxide (16 ml, 2M in water) at room temperature. The reaction mixture was stirred at 25° C. for 1 hour. The pH of the mixture was adjusted to 5 with hydrochloric acid (1M). The mixture was extracted with ethyl acetate. The organic phase was washed with brine, dried over anhydrous sodium sulfate, filtered and concentrated to give a residue. The residue was purified by preparative HPLC (Instrument: Shimadzu LC-20AP; Column: Phenomenex luna C18 250*50 mm*10 μm; eluent A: 0.225% formic acid in water, eluent B: acetonitrile; gradient: 0-28 min 40-70% B; flow 100 ml/min; temperature: room temperature; Detector: UV 220/254 nm) to give (2S)-2-{[(benzyloxy)carbonyl]amino}-3-(1-methoxy-2-naphthyl)propanoic acid (4.98 g, 99% purity, 60% yield) as a white solid.

LC-MS (Method D): Rₜ=0.914 min; MS (ESIpos): m/z=402.1 [M+23]⁺.

¹H NMR (400 MHz, DMSO-d₆): δ [ppm]=8.02 (d, 1H), 7.92 (d, 1H), 7.70 (d, 1H), 7.63 (d, 2H), 7.62-7.61 (m, 2H), 7.43 (d, 1H), 7.35-7.07 (m, 5H), 4.98 (s, 2H), 4.40-4.34 (m, 2H), 3.89 (s, 3H), 3.39-3.34 (m, 1H), 3.02-2.96 (m, 1H).

Intermediate 142

### tri-tert-butyl (5R,12S,16S)-5-[(1-methoxynaphthalen-2-yl)methyl]-3,6,14-trioxo-1-phenyl-2-oxa-4,7,13,15-tetraazaoctadecane-12,16,18-tricarboxylate

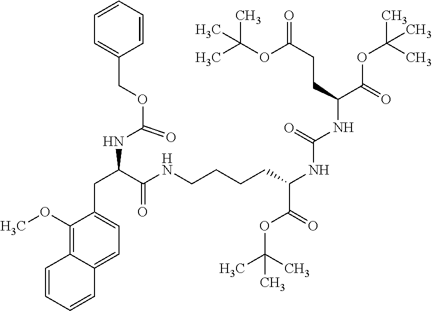 [SMILES: COc1c(C[C@@H](NC(=O)OCc2ccccc2)C(=O)NCCCC[C@H](NC(=O)N[C@@H](CCC(=O)OC(C)(C)C)C(=O)OC(C)(C)C)C(=O)OC(C)(C)C)ccc2ccccc12]

di-tert-butyl N-{[(2S)-6-amino-1-tert-butoxy-1-oxohexan-2-yl]carbamoyl}-L-glutamate (918 mg, 1.88 mmol) and (2R)-2-{[(benzyloxy)carbonyl]amino}-3-(1-methoxynaphthalen-2-yl)propanoic acid (750 mg, 1.98 mmol) were solubilised in DMF (14 ml), 4-methylmorpholine (620 μl, 5.6 mmol, CAS-RN: 109-02-4) and HATU (859 mg, 2.26 mmol) were added and the mixture was stirred under argon for 2 h at rt. The residue was diluted with DCM/propan-2-ol, washed with water and brine. The organic layer was dried and evaporated. The residue was purified by preparative HPLC (C18, acetonitrile/water with 0.1% formic acid) to give 380 mg (95% purity, 23% yield) of the target compound.

LC-MS (Method 1): Rt=1.57 min; MS (ESIpos): m/z=850 [M+H]⁺

Intermediate 143

### di-tert-butyl (2S)-2-({[(2R)-6-{[(2S)-2-amino-3-(1-methoxynaphthalen-2-yl)propanoyl]amino}-1-tert-butoxy-1-oxohexan-2-yl]carbamoyl}amino)pentanedioate

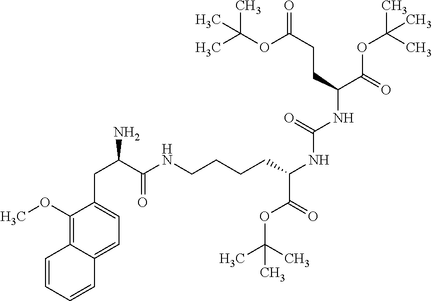 [SMILES: COc1c(C[C@@H](N)C(=O)NCCCC[C@H](NC(=O)N[C@@H](CCC(=O)OC(C)(C)C)C(=O)OC(C)(C)C)C(=O)OC(C)(C)C)ccc2ccccc12]

tri-tert-butyl (5R,12S,16S)-5-[(1-methoxynaphthalen-2-yl)methyl]-3,6,14-trioxo-1-phenyl-2-oxa-4,7,13,15-tetraazaoctadecane-12,16,18-tricarboxylate (380 mg, 448 μmol) was solubilised in MeOH (2.7 ml), Palladium on carbon (47.6 mg, 10% purity, 44.8 μmol) was added and the mixture was purged with hydrogen. The mixture was stirred at rt under hydrogen atmosphere. The mixture was filtered over Celite, washed with MeOH and DCM and evaporated to give 228 mg (97% purity, 69% yield) of the target compound.

LC-MS (Method 1): Rt=1.25 min; MS (ESIpos): m/z=716 [M+H]⁺

Intermediate 144

### tri-tert-butyl (3R,10S,14S)-1-[(1r,4S)-4-({[(benzyloxy)carbonyl]amino}methyl)cyclohexyl]-3-[(1-methoxynaphthalen-2-yl)methyl]-1,4,12-trioxo-2,5,11,13-tetraazahexadecane-10,14,16-tricarboxylate

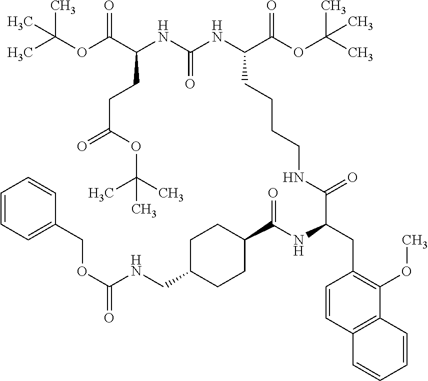 [SMILES: COc1c(C[C@@H](NC(=O)[C@H]2CC[C@H](CNC(=O)OCc3ccccc3)CC2)C(=O)NCCCC[C@H](NC(=O)N[C@@H](CCC(=O)OC(C)(C)C)C(=O)OC(C)(C)C)C(=O)OC(C)(C)C)ccc2ccccc12]

di-tert-butyl (2S)-2-({[(2S)-6-{[(2R)-2-amino-3-(1-methoxynaphthalen-2-yl)propanoyl]amino}-1-tert-butoxy-1-oxohexan-2-yl]carbamoyl}amino)pentanedioate (228 mg, 319 μmol) and (1r,4r)-4-({[(benzyloxy)carbonyl]amino}methyl)cyclohexane-1-carboxylic acid (92.9 mg, 319 μmol) were solubilised in DMF (4.9 ml), 4-methylmorpholine (88 μl, 800 μmol, CAS-RN: 109-02-4) and HATU (146 mg, 383 μmol) were added and the mixture was stirred under argon overnight at rt. The mixture was filtered and purified by preparative HPLC (C18, acetonitrile/water with 0.1% formic acid) to give 95.0 mg (100% purity, 30% yield) of the target compound.

LC-MS (Method 1): Rt=1.57 min; MS (ESIpos): m/z=989 [M+H]⁺

Intermediate 145

### tri-tert-butyl (3R,10S,14S)-1-[(1r,4S)-4-(aminomethyl)cyclohexyl]-3-[(1-methoxynaphthalen-2-yl)methyl]-1,4,12-trioxo-2,5,11,13-tetraazahexadecane-10,14,16-tricarboxylate

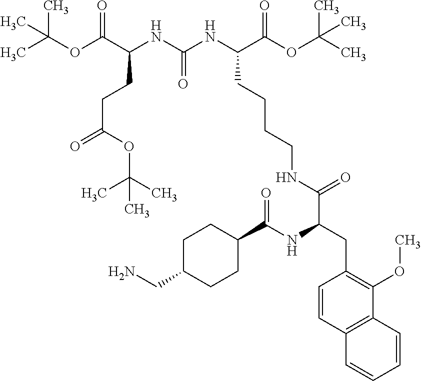 [SMILES: COc1c(C[C@@H](NC(=O)[C@H]2CC[C@H](CN)CC2)C(=O)NCCCC[C@H](NC(=O)N[C@@H](CCC(=O)OC(C)(C)C)C(=O)OC(C)(C)C)C(=O)OC(C)(C)C)ccc2ccccc12]

tri-tert-butyl (3R,10S,14S)-1-[(1r,4S)-4-({[(benzyloxy)carbonyl]amino}methyl)cyclohexyl]-3-[(1-methoxynaphthalen-2-yl)methyl]-1,4,12-trioxo-2,5,11,13-tetraazahexadecane-10,14,16-tricarboxylate (100 mg, 101 μmol) was solubilised in MeOH (200 μl) and THF (410 μl), Palladium on carbon (10.8 mg, 10% purity, 10.1 μmol) was added and the mixture was purged with hydrogen. The mixture was stirred for 2 h at rt under hydrogen atmosphere. The mixture was filtered over Celite, washed with MeOH and evaporated. The residue was purified by preparative HPLC (C18, acetonitrile/water with 0.1% formic acid) to give 110 mg (70% purity, 89% yield) of the target compound.

LC-MS (Method 1): Rt=1.22 min; MS (ESIpos): m/z=855 [M+H]⁺

### Example 33-A

### (3R,10S,14S)-1-[(1r,4S)-4-(aminomethyl)cyclohexyl]-3-[(1-methoxynaphthalen-2-yl)methyl]-1,4,12-trioxo-2,5,11,13-tetraazahexadecane-10,14,16-tricarboxylic acid

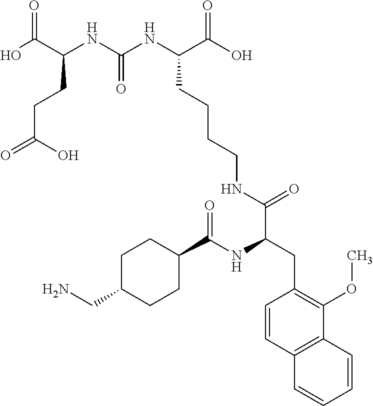 [SMILES: COc1c(C[C@@H](NC(=O)[C@H]2CC[C@H](CN)CC2)C(=O)NCCCC[C@H](NC(=O)N[C@@H](CCC(=O)O)C(=O)O)C(=O)O)ccc2ccccc12]

tri-tert-butyl (3R,10S,14S)-1-[(1r,4S)-4-(aminomethyl)cyclohexyl]-3-[(1-methoxynaphthalen-2-yl)methyl]-1,4,12-trioxo-2,5,11,13-tetraazahexadecane-10,14,16-tricarboxylate (50.0 mg, 58.5 μmol) was stirred in TFA (900 μl, 12 mmol) under argon overnight at rt. The mixture was evaporated and purified by preparative HPLC (C18, acetonitrile/water with 0.1% formic acid) to give 14.0 mg (99% purity, 35% yield) of the target compound.

LC-MS (Method 1): Rt=0.74 min; MS (ESIpos): m/z=687 [M+H]⁺

¹H-NMR (400 MHz, DMSO-d6) δ[ppm]: 0.847 (2.04), 0.878 (2.25), 0.910 (0.98), 1.081 (0.56), 1.113 (1.26), 1.146 (1.40), 1.183 (1.54), 1.230 (2.81), 1.258 (2.95), 1.302 (1.89), 1.317 (1.82), 1.337 (1.75), 1.353 (1.89), 1.369 (1.61), 1.401 (2.04), 1.422 (2.11), 1.433 (2.04), 1.511 (1.61), 1.541 (1.40), 1.608 (2.18), 1.630 (2.53), 1.642 (2.81), 1.680 (2.60), 1.714 (1.33), 1.774 (2.25), 1.795 (2.25), 2.034 (0.91), 2.063 (1.68), 2.074 (3.23), 2.094 (0.91), 2.125 (0.63), 2.145 (0.84), 2.161 (1.68), 2.180 (1.61), 2.193 (1.12), 2.214 (1.19), 2.236 (2.04), 2.258 (1.19), 2.272 (1.12), 2.294 (0.56), 2.323 (3.09), 2.327 (4.28), 2.332 (3.23), 2.522 (16.00), 2.556 (1.75), 2.569 (2.88), 2.587 (4.35), 2.603 (2.67), 2.618 (1.12), 2.635 (0.77), 2.665 (3.23), 2.669 (4.35), 2.673 (3.30), 2.910 (1.47), 2.935 (2.39), 2.945 (2.60), 2.969 (3.02), 3.077 (1.47), 3.093 (1.82), 3.110 (1.61), 3.174 (2.53), 3.187 (2.88), 3.208 (2.88), 3.221 (2.95), 3.335 (7.23), 3.934 (3.30), 3.946 (2.53), 4.509 (0.91), 4.532 (1.75), 4.545 (1.75), 4.568 (0.91), 6.119 (1.26), 6.444 (1.05), 7.369 (4.14), 7.391 (4.84), 7.454 (1.47), 7.471 (3.30), 7.487 (2.67), 7.491 (2.67), 7.502 (2.53), 7.506 (2.60), 7.523 (3.16), 7.540 (1.47), 7.575 (4.70), 7.596 (3.72), 7.822 (1.12), 7.867 (3.86), 7.886 (3.44), 7.988 (3.72), 8.008 (3.37), 8.189 (0.77), 8.222 (1.47).

Intermediate 146

### tri-tert-butyl (3R,10S,14S)-3-[(1-methoxynaphthalen-2-yl)methyl]-1,4,12-trioxo-1-[(1r,4S)-4-({2-[4,7,10-tris(2-tert-butoxy-2-oxoethyl)-1,4,7,10-tetraazacyclododecan-1-yl]acetamido}methyl)cyclohexyl]-2,5,11,13-tetraazahexadecane-10,14,16-tricarboxylate

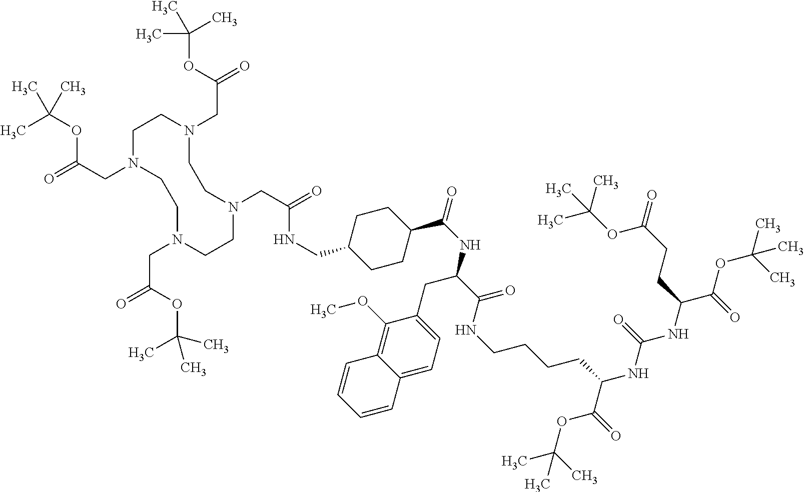 [SMILES: COc1c(C[C@@H](NC(=O)[C@H]2CC[C@H](CNC(=O)CN3CCN(CC(=O)OC(C)(C)C)CCN(CC(=O)OC(C)(C)C)CCN(CC(=O)OC(C)(C)C)CC3)CC2)C(=O)NCCCC[C@H](NC(=O)N[C@@H](CCC(=O)OC(C)(C)C)C(=O)OC(C)(C)C)C(=O)OC(C)(C)C)ccc2ccccc12]

[4,7,10-tris(2-tert-butoxy-2-oxoethyl)-1,4,7,10-tetraazacyclododecan-1-yl]acetic acid (207 mg, 361 μmol; CAS-RN:[137076-54-1]), [(1H-benzotriazol-1-yl)oxy](dimethylamino)-N,N-dimethylmethaniminium hexafluoridophosphate(1−) (135 mg, 356 μmol; CAS-RN:94790-37-1) and N,N-diisopropylethylamine (46 μl, 270 μmol) were stirred in DMF (1.4 ml) for 10 min at rt. tri-tert-butyl (3R,10S,14S)-1-[(1r,4S)-4-(aminomethyl)cyclohexyl]-3-[(1-methoxynaphthalen-2-yl)methyl]-1,4,12-trioxo-2,5,11,13-tetraazahexadecane-10,14,16-tricarboxylate (110 mg, 70% purity, 90.2 μmol) was added and the mixture was stirred at rt. The mixture was evaporated and purified by preparative HPLC (C18, acetonitrile/water with 0.1% formic acid) to give 155.0 mg (80% purity, 98% yield) of the target compound.

LC-MS (Method 1): Rt=1.28 min; MS (ESIpos): m/z=1410 [M+H]⁺

### Example 33-B

### (3R,10S,14S)-3-[(1-methoxynaphthalen-2-yl)methyl]-1,4,12-trioxo-1-[(1r,4S)-4-({2-[4,7,10-tris(carboxymethyl)-1,4,7,10-tetraazacyclododecan-1-yl]acetamido}methyl)cyclohexyl]-2,5,11,13-tetraazahexadecane-10,14,16-tricarboxylic acid

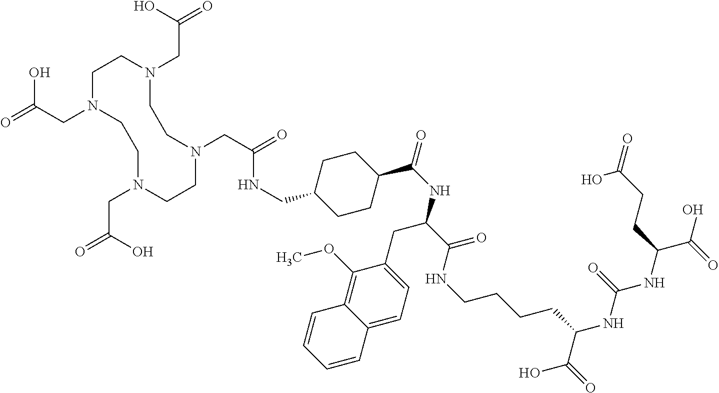 [SMILES: COc1c(C[C@@H](NC(=O)[C@H]2CC[C@H](CNC(=O)CN3CCN(CC(=O)O)CCN(CC(=O)O)CCN(CC(=O)O)CC3)CC2)C(=O)NCCCC[C@H](NC(=O)N[C@@H](CCC(=O)O)C(=O)O)C(=O)O)ccc2ccccc12]

tri-tert-butyl (3R,10S,14S)-3-[(1-methoxynaphthalen-2-yl)methyl]-1,4,12-trioxo-1-[(1r,4S)-4-({2-[4,7,10-tris(2-tert-butoxy-2-oxoethyl)-1,4,7,10-tetraazacyclododecan-1-yl]acetamido}methyl)cyclohexyl]-2,5,11,13-tetraazahexadecane-10,14,16-tricarboxylate (195 mg, 138 μmol) was solubilised in DCM (450 μl), TFA (210 μl, 2.8 mmol) was added and the mixture was stirred under argon overnight at rt. The mixture was evaporated and purified by preparative HPLC (C18, acetonitrile/water with 0.1% formic acid) to give 37.0 mg (100% purity, 25% yield) of the target compound.

LC-MS (Method 1): Rₜ=0.73 min; MS (ESIneg): m/z=1071 [M−H]⁻

¹H-NMR (400 MHz, DMSO-d6) δ[ppm]: 0.802 (0.86), 0.833 (1.04), 0.852 (0.52), 1.116 (0.52), 1.145 (0.52), 1.237 (2.11), 1.301 (0.91), 1.317 (1.29), 1.333 (1.25), 1.352 (1.08), 1.413 (0.39), 1.432 (0.52), 1.449 (0.56), 1.468 (0.47), 1.510 (0.60), 1.541 (0.65), 1.600 (0.91), 1.634 (1.16), 1.663 (1.12), 1.699 (0.99), 1.718 (0.78), 1.735 (0.52), 1.885 (0.52), 1.902 (0.56), 2.049 (0.65), 2.075 (0.78), 2.085 (0.69), 2.208 (0.86), 2.223 (1.34), 2.245 (1.29), 2.262 (0.69), 2.318 (1.04), 2.323 (1.98), 2.327 (2.72), 2.332 (1.90), 2.336 (0.91), 2.456 (0.52), 2.518 (9.75), 2.523 (6.68), 2.649 (2.11), 2.660 (2.54), 2.665 (3.28), 2.669 (3.71), 2.673 (2.63), 2.678 (1.42), 2.907 (2.03), 2.979 (5.05), 2.997 (4.40), 3.097 (2.20), 3.182 (1.25), 3.203 (1.29), 3.216 (1.34), 3.339 (3.58), 3.880 (16.00), 3.957 (0.43), 3.976 (0.82), 3.989 (0.86), 4.008 (0.43), 4.073 (0.78), 4.087 (0.78), 4.107 (0.39), 4.517 (0.73), 4.531 (0.73), 6.295 (0.95), 6.314 (1.55), 6.335 (0.91), 7.359 (1.94), 7.380 (2.24), 7.451 (0.65), 7.468 (1.51), 7.488 (1.21), 7.504 (1.21), 7.522 (1.42), 7.539 (0.69), 7.578 (1.94), 7.600 (1.60), 7.867 (2.37), 7.886 (1.94), 7.989 (1.77), 8.010 (1.73), 8.075 (0.95), 8.138 (0.47).

### Example 33-B 227Th

### (3R,10S,14S)-3-[(1-methoxynaphthalen-2-yl)methyl]-1,4,12-trioxo-1-{5-[(2-{4,7,10-tris[(carboxy-kappaO)methyl]-1,4,7,10-tetraazacyclododecan-1-yl-kappa²N¹,N⁴}acetamido)methyl]pyridin-2-yl}-2,5,11,13-tetraazahexadecane-10,14,16-tricarboxylato(3−)(²²⁷Th)Thorium

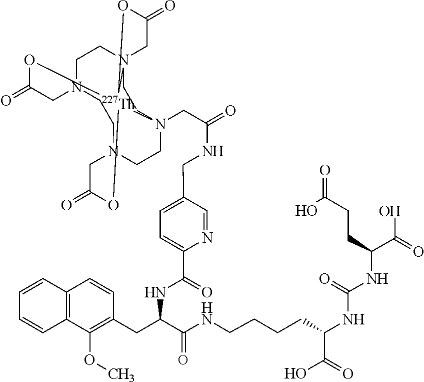

(3S,10S,14S)-3-[(1-methoxynaphthalen-2-yl)methyl]-1,4,12-trioxo-1-[(1r,4S)-4-({2-[4,7,10-tris(carboxymethyl)-1,4,7,10-tetraazacyclododecan-1-yl]acetamido}methyl)cyclohexyl]-2,5,11,13-tetraazahexadecane-10,14,16-tricarboxylic acid was dissolved as 10 mM solution in DMSO and diluted to a 250 μM solution in 400 mM sodium acetate buffer (pH 5) containing 0.5 mg/mL pABA. 1.07 μl of this solution was used in a 40 μl labeling reaction. Thorium-227 in 400 mM sodium acetate buffer (pH 5) plus 20% EtOH was added giving a specific activity of 0.375 MBq/nmol and a RAC of 2.5 MBq/mL. The mixture was incubated at 95° C. for 60 min. The labelling efficiency was determined to be 96.3% by iTLC.

Intermediate 147

### tri-tert-butyl (3R,10S,14S)-1-{5-[({[(9H-fluoren-9-yl)methoxy]carbonyl}amino)methyl]pyridin-2-yl}-3-[(1-methoxynaphthalen-2-yl)methyl]-1,4,12-trioxo-2,5,11,13-tetraazahexadecane-10,14,16-tricarboxylate

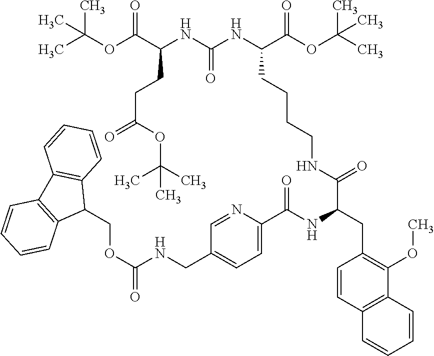 [SMILES: COc1c(C[C@@H](NC(=O)c2ccc(CNC(=O)OCC3c4ccccc4-c4ccccc43)cn2)C(=O)NCCCC[C@H](NC(=O)N[C@@H](CCC(=O)OC(C)(C)C)C(=O)OC(C)(C)C)C(=O)OC(C)(C)C)ccc2ccccc12]

di-tert-butyl (2S)-2-({[(2S)-6-{[(2R)-2-amino-3-(1-methoxynaphthalen-2-yl)propanoyl]amino}-1-tert-butoxy-1-oxohexan-2-yl]carbamoyl}amino)pentanedioate (295 mg, 413 μmol) and 5-[({[(9H-fluoren-9-yl)methoxy]carbonyl}amino)methyl]pyridine-2-carboxylic acid (154 mg, 413 μmol) were solubilised in DMF (6.3 ml), 4-methylmorpholine (110 μl, 1.0 mmol, CAS-RN: 109-02-4) and HATU (188 mg, 495 μmol) were added and the mixture was stirred under argon overnight at rt. The mixture was evaporated and purified by preparative HPLC (C18, acetonitrile/water with 0.1% formic acid) to give 160.0 mg (80% purity, 29% yield) of the target compound.

LC-MS (Method 1): Rt=1.63 min; MS (ESIpos): m/z=1072 [M+H]⁺

Intermediate 148

### tri-tert-butyl (3R,10S,14S)-1-[5-(aminomethyl)pyridin-2-yl]-3-[(1-methoxynaphthalen-2-yl)methyl]-1,4,12-trioxo-2,5,11,13-tetraazahexadecane-10,14,16-tricarboxylate

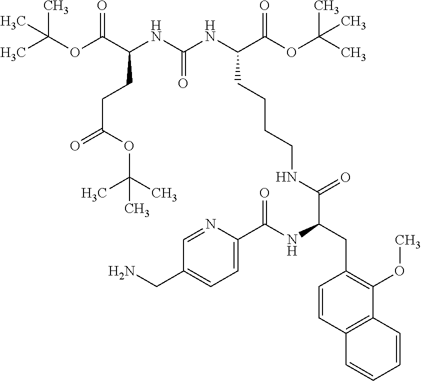 [SMILES: COc1c(C[C@@H](NC(=O)c2ccc(CN)cn2)C(=O)NCCCC[C@H](NC(=O)N[C@@H](CCC(=O)OC(C)(C)C)C(=O)OC(C)(C)C)C(=O)OC(C)(C)C)ccc2ccccc12]

tri-tert-butyl (3R,10S,14S)-1-{5-[({[(9H-fluoren-9-yl)methoxy]carbonyl}amino)methyl]pyridin-2-yl}-3-[(1-methoxynaphthalen-2-yl)methyl]-1,4,12-trioxo-2,5,11,13-tetraazahexadecane-10,14,16-tricarboxylate (160 mg, 149 μmol) was solubilised in DMF (2.3 ml), piperidine (300 μl, 3.0 mmol) was added and the mixture was stirred under argon overnight at rt. The mixture was evaporated and purified by preparative HPLC (C18, acetonitrile/water with 0.1% formic acid) to give 110 mg (100% purity, 87% yield) of the target compound.

LC-MS (Method 1): Rt=1.25 min; MS (ESIpos): m/z=850 [M+H]⁺

¹H-NMR (400 MHz, DMSO-d6) δ[ppm]: 1.358 (0.78), 1.370 (7.12), 1.379 (16.00), 2.518 (0.58), 3.224 (0.41), 3.242 (0.42), 3.866 (1.06), 3.890 (2.63), 7.383 (0.40), 7.906 (0.81), 8.263 (0.60), 8.591 (0.41).

Intermediate 149

### tri-tert-butyl (3R,10S,14S)-3-[(1-methoxynaphthalen-2-yl)methyl]-1,4,12-trioxo-1-[5-({2-[4,7,10-tris(2-tert-butoxy-2-oxoethyl)-1,4,7,10-tetraazacyclododecan-1-yl]acetamido}methyl)pyridin-2-yl]-2,5,11,13-tetraazahexadecane-10,14,16-tricarboxylate

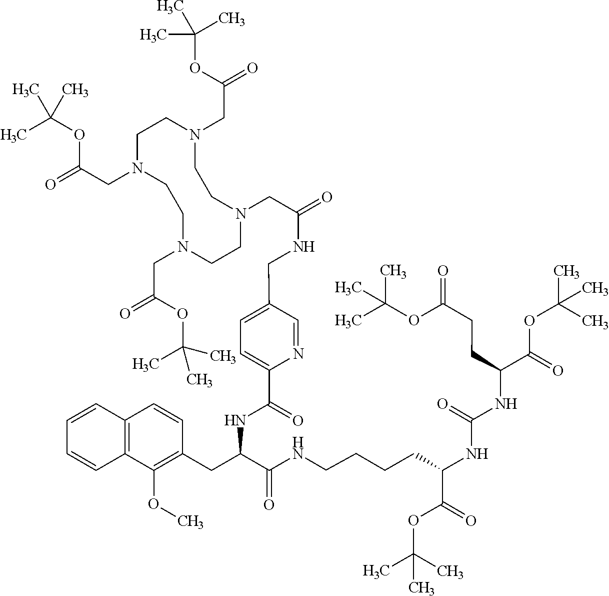 [SMILES: COc1c(C[C@@H](NC(=O)c2ccc(CNC(=O)CN3CCN(CC(=O)OC(C)(C)C)CCN(CC(=O)OC(C)(C)C)CCN(CC(=O)OC(C)(C)C)CC3)cn2)C(=O)NCCCC[C@H](NC(=O)N[C@@H](CCC(=O)OC(C)(C)C)C(=O)OC(C)(C)C)C(=O)OC(C)(C)C)ccc2ccccc12]

[4,7,10-tris(2-tert-butoxy-2-oxoethyl)-1,4,7,10-tetraazacyclododecan-1-yl]acetic acid (108 mg, 188 μmol; CAS-RN:[137076-54-1]), [(1H-benzotriazol-1-yl)oxy](dimethylamino)-N,N-dimethylmethaniminium hexafluoridophosphate(1−) (70.6 mg, 186 μmol; CAS-RN:94790-37-1) and N,N-diisopropylethylamine (23 μl, 140 μmol) were stirred in DMF (1.8 ml) for 10 min at rt. tri-tert-butyl (3R,10S,14S)-1-[5-(aminomethyl)pyridin-2-yl]-3-[(1-methoxynaphthalen-2-yl)methyl]-1,4,12-trioxo-2,5,11,13-tetraazahexadecane-10,14,16-tricarboxylate (40.0 mg, 47.1 μmol) was added and the mixture was stirred overnight at rt. The mixture was evaporated and purified by preparative HPLC (C18, acetonitrile/water with 0.1% formic acid) to give 41.0 mg (100% purity, 62% yield) of the target compound.

¹H-NMR (400 MHz, DMSO-d6) δ[ppm]: 1.315 (2.41), 1.324 (2.68), 1.370 (6.86), 1.380 (16.00), 1.396 (2.77), 1.437 (2.21), 2.518 (1.96), 2.522 (1.23), 2.669 (0.49), 3.873 (1.66), 3.881 (0.61).

### Example 33a-B

### (3R,10S,14S)-3-[(1-methoxynaphthalen-2-yl)methyl]-1,4,12-trioxo-1-[5-({2-[4,7,10-tris(carboxymethyl)-1,4,7,10-tetraazacyclododecan-1-yl]acetamido}methyl)pyridin-2-yl]-2,5,11,13-tetraazahexadecane-10,14,16-tricarboxylic acid

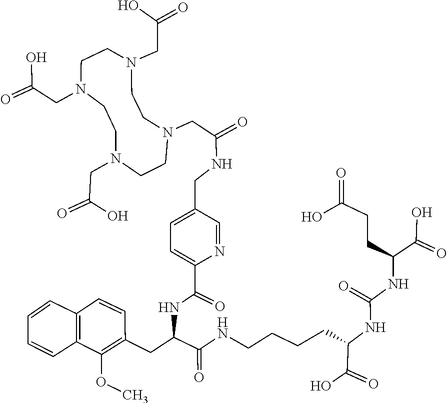 [SMILES: COc1c(C[C@@H](NC(=O)c2ccc(CNC(=O)CN3CCN(CC(=O)O)CCN(CC(=O)O)CCN(CC(=O)O)CC3)cn2)C(=O)NCCCC[C@H](NC(=O)N[C@@H](CCC(=O)O)C(=O)O)C(=O)O)ccc2ccccc12]

tri-tert-butyl (3R,10S,14S)-3-[(1-methoxynaphthalen-2-yl)methyl]-1,4,12-trioxo-1-[5-({2-[4,7,10-tris(2-tert-butoxy-2-oxoethyl)-1,4,7,10-tetraazacyclododecan-1-yl]acetamido}methyl)pyridin-2-yl]-2,5,11,13-tetraazahexadecane-10,14,16-tricarboxylate (40.0 mg, 28.5 μmol) was solubilised in DCM (920 μl), TFA (440 μl, 5.7 mmol) was added and the mixture was stirred under argon over the weekend at rt. The mixture was evaporated and purified by preparative HPLC (C18, acetonitrile/water with 0.1% formic acid) to give 6.00 mg (98% purity, 19% yield) of the target compound.

LC-MS (Method 1): Rt=0.72 min; MS (ESIpos): m/z=1068 [M+H]⁺

¹H-NMR (400 MHz, DMSO-d6) δ[ppm]: 1.206 (0.77), 1.225 (1.40), 1.241 (1.35), 1.259 (0.68), 1.293 (0.44), 1.309 (0.77), 1.328 (0.98), 1.347 (0.89), 1.364 (0.58), 1.442 (0.51), 1.458 (0.63), 1.477 (0.49), 1.560 (0.44), 1.577 (0.56), 1.595 (0.54), 1.673 (0.42), 1.688 (0.56), 1.708 (0.68), 1.723 (0.51), 1.866 (0.44), 1.884 (0.61), 1.901 (0.65), 2.202 (0.95), 2.217 (1.63), 2.224 (1.21), 2.239 (1.58), 2.257 (0.84), 2.336 (0.51), 2.460 (0.58), 2.463 (0.68), 2.518 (5.22), 2.522 (3.35), 2.601 (1.84), 2.660 (0.65), 2.918 (2.68), 3.023 (5.40), 3.114 (2.82), 3.191 (1.40), 3.225 (2.47), 3.245 (3.42), 3.259 (3.21), 3.473 (4.19), 3.682 (0.42), 3.900 (16.00), 3.962 (0.54), 3.983 (0.98), 3.996 (1.00), 4.015 (0.51), 4.055 (0.56), 4.069 (0.72), 4.075 (1.09), 4.088 (1.09), 4.096 (0.68), 4.109 (0.54), 4.383 (1.82), 4.397 (1.79), 4.762 (0.47), 4.784 (0.86), 4.799 (0.91), 4.820 (0.44), 6.284 (1.44), 6.304 (2.49), 6.325 (1.35), 7.362 (2.31), 7.383 (2.63), 7.439 (0.68), 7.443 (0.70), 7.459 (1.58), 7.476 (1.28), 7.480 (1.28), 7.492 (1.21), 7.496 (1.26), 7.513 (1.54), 7.516 (1.30), 7.530 (0.75), 7.533 (0.72), 7.562 (2.35), 7.584 (1.89), 7.835 (1.68), 7.843 (1.96), 7.855 (2.28), 7.862 (1.84), 7.931 (1.23), 7.951 (0.84), 7.985 (1.68), 8.006 (1.54), 8.112 (0.58), 8.126 (1.14), 8.139 (0.79), 8.616 (2.07), 8.728 (1.26), 8.751 (1.40), 8.772 (1.09), 8.786 (0.56).

### Example 33a-B-227Th

### (3R,10S,14S)-3-[(1-methoxynaphthalen-2-yl)methyl]-1,4,12-trioxo-1-{5-[(2-{4,7,10-tris[(carboxy-kappaO)methyl]-1,4,7,10-tetraazacyclododecan-1-yl-kappa²N¹,N⁴}acetamido)methyl]pyridin-2-yl}-2,5,11,13-tetraazahexadecane-10,14,16-tricarboxylato(3−)(²²⁷Th)Thorium

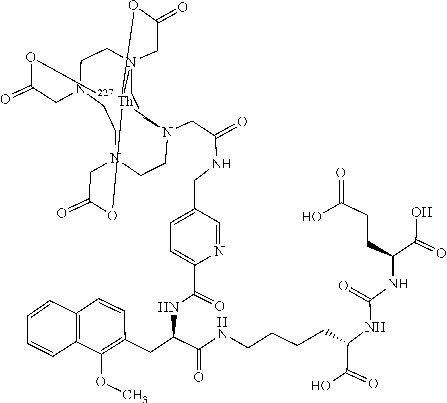

(3R,10S,14S)-3-[(1-methoxynaphthalen-2-yl)methyl]-1,4,12-trioxo-1-[5-({2-[4,7,10-tris(carboxymethyl)-1,4,7,10-tetraazacyclododecan-1-yl]acetamido}methyl)pyridin-2-yl]-2,5,11,13-tetraazahexadecane-10,14,16-tricarboxylic acid was dissolved as 10 mM solution in DMSO and diluted to a 250 μM solution in 400 mM sodium acetate buffer (pH 5) containing 0.5 mg/mL pABA. 1.07 μl of this solution was used in a 40 μl labeling reaction. Thorium-227 in 400 mM sodium acetate buffer (pH 5) plus 20% EtOH was added giving a specific activity of 0.375 MBq/nmol and a RAC of 2.5 MBq/mL. The mixture was incubated at 95° C. for 60 min. The labelling efficiency was determined to be 94.4% by iTLC.

#34 Linker

Intermediate 150

### tri-tert-butyl (3S,10S,14S)-1-{5-[({[(9H-fluoren-9-yl)methoxy]carbonyl}amino)methyl]pyridin-2-yl}-1,4,12-trioxo-3-[(quinolin-2-yl)methyl]-2,5,11,13-tetraazahexadecane-10,14,16-tricarboxylate

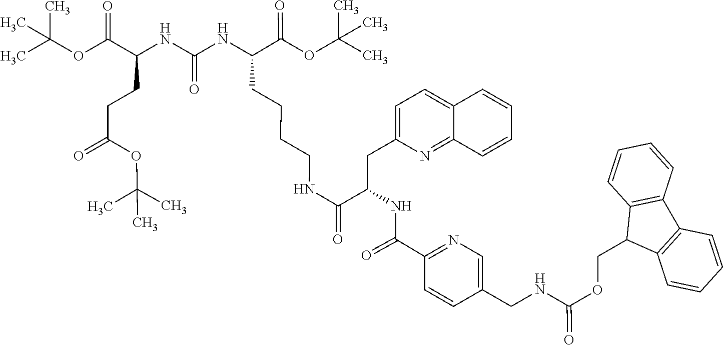 [SMILES: CC(C)(C)OC(=O)CC[C@H](NC(=O)N[C@@H](CCCCNC(=O)[C@H](Cc1ccc2ccccc2n1)NC(=O)c1ccc(CNC(=O)OCC2c3ccccc3-c3ccccc32)cn1)C(=O)OC(C)(C)C)C(=O)OC(C)(C)C]

di-tert-butyl (2S)-2-({[(2S)-6-{[(2S)-2-amino-3-(quinolin-2-yl)propanoyl]amino}-1-tert-butoxy-1-oxohexan-2-yl]carbamoyl}amino)pentanedioate (83.0 mg, 121 μmol) and (2S)-5-[({[(9H-fluoren-9-yl)methoxy]carbonyl}amino)methyl]pyridine-2-carboxylic acid (64.0 mg, 85% purity, 145 μmol) were solubilised in DMF (940 μl), 4-methylmorpholine (79 μl, 480 μmol, CAS-RN: 109-02-4) and HATU (102 mg, 182 μmol) were added and the mixture was stirred under argon overnight at rt. The mixture was evaporated and purified by preparative HPLC (C18, acetonitrile/water with 0.1% formic acid) to give 82.0 mg (65% yield) of the target compound.

LC-MS (Method 1): Rt=1.57 min; MS (ESIpos): m/z=1043 [M+H]⁺ NMR:

Intermediate 151

### tri-tert-butyl (3S,10S,14S)-1-[5-(aminomethyl)pyridin-2-yl]-1,4,12-trioxo-3-[(quinolin-2-yl)methyl]-2,5,11,13-tetraazahexadecane-10,14,16-tricarboxylate

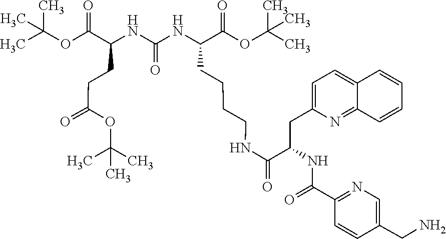 [SMILES: CC(C)(C)OC(=O)CC[C@H](NC(=O)N[C@@H](CCCCNC(=O)[C@H](Cc1ccc2ccccc2n1)NC(=O)c1ccc(CN)cn1)C(=O)OC(C)(C)C)C(=O)OC(C)(C)C]

tri-tert-butyl (3S,10S,14S)-1-{5-[({[(9H-fluoren-9-yl)methoxy]carbonyl}amino)methyl]pyridin-2-yl}-1,4,12-trioxo-3-[(quinolin-2-yl)methyl]-2,5,11,13-tetraazahexadecane-10,14,16-tricarboxylate (80.0 mg, 76.8 μmol) was solubilised in DMF (1.2 ml), piperidine (150 μl, 1.5 mmol) was added and the mixture was stirred under argon overnight at rt. The mixture was evaporated and purified by preparative HPLC (C18, acetonitrile/water with 0.1% formic acid) to give 37.0 mg (97% purity, 57% yield) of the target compound.

LC-MS (Method 1): Rt=1.14 min; MS (ESIpos): m/z=821 [M+H]⁺

¹H-NMR (400 MHz, DMSO-d6) δ[ppm]: 1.368 (9.95), 1.376 (14.05), 1.379 (16.00), 2.323 (0.51), 2.327 (0.67), 2.665 (0.51), 2.669 (0.69), 3.389 (0.78), 3.405 (0.62), 3.859 (1.63), 7.399 (0.59), 7.421 (0.60), 7.898 (0.45), 7.912 (1.51), 7.914 (1.48), 7.951 (0.45), 8.227 (0.55), 8.232 (0.54), 8.248 (0.48), 8.626 (0.64).

### Example 34-A

### N⁶—{N-[5-(aminomethyl)pyridine-2-carbonyl]-3-(quinolin-2-yl)-L-alanyl}-N²-{[(1S)-1,3-dicarboxypropyl]carbamoyl}-L-lysine

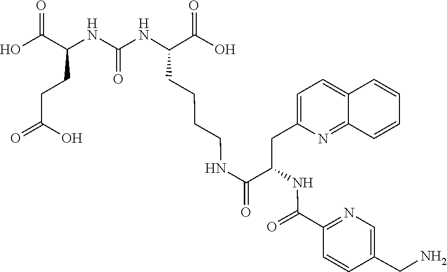 [SMILES: NCc1ccc(C(=O)N[C@@H](Cc2ccc3ccccc3n2)C(=O)NCCCC[C@H](NC(=O)N[C@@H](CCC(=O)O)C(=O)O)C(=O)O)nc1]

tri-tert-butyl (3S,10S,14S)-1-[5-(aminomethyl)pyridin-2-yl]-1,4,12-trioxo-3-[(quinolin-2-yl)methyl]-2,5,11,13-tetraazahexadecane-10,14,16-tricarboxylate (10.0 mg, 12.2 μmol) was solubilised in DCM (390 μl), TFA (190 μl, 2.4 mmol) was added and the mixture was stirred under argon overnight at rt. The mixture was evaporated and purified by preparative HPLC (C18, acetonitrile/water with 0.1% formic acid) to give 3.50 mg (98% purity, 43% yield) of the target compound.

LC-MS (Method 1): Rt=0.55 min; MS (ESIpos): m/z=653 [M+H]⁺

¹H-NMR (400 MHz, DMSO-d6) δ[ppm]: 1.208 (1.47), 1.230 (1.88), 1.274 (0.71), 1.290 (1.18), 1.307 (1.59), 1.326 (1.41), 1.343 (0.88), 1.379 (0.65), 1.392 (0.76), 1.412 (0.76), 1.428 (0.65), 1.531 (0.88), 1.549 (0.71), 1.657 (0.41), 1.670 (0.76), 1.690 (1.00), 1.705 (0.76), 1.724 (0.71), 1.744 (1.00), 1.763 (1.12), 1.779 (0.76), 1.796 (0.59), 2.151 (0.47), 2.174 (1.24), 2.194 (1.41), 2.201 (1.29), 2.222 (2.00), 2.242 (1.00), 2.259 (0.76), 2.332 (2.53), 2.336 (1.24), 2.518 (12.18), 2.522 (7.94), 2.678 (1.29), 2.686 (0.65), 2.966 (1.12), 2.982 (1.53), 2.999 (2.06), 3.013 (2.24), 3.025 (2.29), 3.039 (2.29), 3.057 (1.94), 3.074 (1.71), 3.407 (16.00), 3.423 (15.24), 3.917 (1.24), 3.938 (2.18), 3.951 (3.00), 3.970 (2.18), 4.033 (6.47), 4.070 (0.59), 4.904 (0.76), 4.920 (1.76), 4.941 (1.71), 4.957 (0.71), 6.123 (1.06), 6.140 (1.00), 6.334 (1.88), 6.354 (1.82), 7.421 (4.24), 7.442 (4.12), 7.525 (1.29), 7.527 (1.24), 7.545 (2.53), 7.562 (1.59), 7.565 (1.53), 7.710 (1.35), 7.714 (1.35), 7.731 (2.41), 7.735 (1.65), 7.749 (1.29), 7.752 (1.29), 7.901 (2.41), 7.920 (2.24), 7.951 (8.29), 7.955 (6.06), 7.978 (2.47), 8.080 (1.00), 8.094 (1.94), 8.108 (0.94), 8.233 (3.35), 8.254 (3.12), 8.704 (3.59), 9.210 (2.47), 9.231 (2.35).

Intermediate 152 Monomer

### 2,2′-{({3-[(2-{[1-(2-{[(6-{[(7S,11S,18S)-7,11-bis(tert-butoxycarbonyl)-2,2-dimethyl-4,9,17-trioxo-19-(quinolin-2-yl)-3-oxa-8,10,16-triazanonadecan-18-yl]carbamoyl}pyridin-3-yl)methyl]amino}-2-oxoethyl)-3-hydroxy-6-methyl-2-oxo-1,2-dihydropyridine-4-carbonyl]amino}ethyl)(2-{[1-(carboxymethyl)-3-hydroxy-6-methyl-2-oxopyridine-4(2H)-carbonyl]amino}ethyl)amino]propyl}azanediyl)bis[(ethane-2,1-diyl)carbamoyl(3-hydroxy-6-methyl-2-oxopyridine-4,1(2H)-diyl)]}diacetic acid

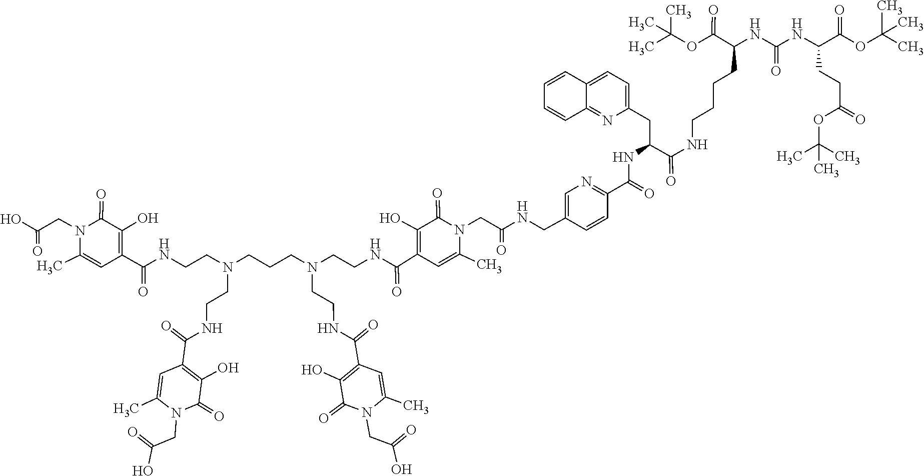 [SMILES: Cc1cc(C(=O)NCCN(CCCN(CCNC(=O)c2cc(C)n(CC(=O)O)c(=O)c2O)CCNC(=O)c2cc(C)n(CC(=O)NCc3ccc(C(=O)N[C@@H](Cc4ccc5ccccc5n4)C(=O)NCCCC[C@H](NC(=O)N[C@@H](CCC(=O)OC(C)(C)C)C(=O)OC(C)(C)C)C(=O)OC(C)(C)C)nc3)c(=O)c2O)CCNC(=O)c2cc(C)n(CC(=O)O)c(=O)c2O)c(O)c(=O)n1CC(=O)O]

2,2′,2″,2′″-(propane-1,3-diylbis{nitrilobis[(ethane-2,1-diyl)carbamoyl(3-hydroxy-6-methyl-2-oxopyridine-4,1(2H)-diyl)]})tetraacetic acid (3.50 mg, 3.23 μmol) and tri-tert-butyl (3S,10S,14S)-1-[5-(aminomethyl)pyridin-2-yl]-1,4,12-trioxo-3-[(quinolin-2-yl)methyl]-2,5,11,13-tetraazahexadecane-10,14,16-tricarboxylate (2.65 mg, 3.23 μmol) are dissolved in NMP (610 μL). DIPEA (3.8 μL, 22 μmol) is added. PyAOP (2.1 mg, 4.04 μmol) in NMP 170 μL is added. Reaction mix is diluted with 20% ACN/water/0.1% TFA (8 mL) and the products purified by preparative HPLC (RP-HPLC using Äkta pure system, Column: Phenomenex Luna 5 μm C18(2) 100 Å, 250×21.2 mm, Mobile phase: Water/0.1% TFA; ACN Gradient: 20-70% B over 40 min Flow: 10 mL/min Detection: UV 280/335 nm, tR product: 28 min) afforded 1.6 mg (26% yield) of the target compound.

LC-MS (Method K, gradient: 10-70% B over 3 min): Rₜ=2.14 min; MS (ESIpos): m/z=1885.0 [M+H]⁺

Intermediate 153 Dimer

### 2-[4-[2-[2-[[1-[2-[[6-[[(1S)-2-[[(5S)-6-tert-butoxy-5-[[(1S)-4-tert-butoxy-1-tert-butoxycarbonyl-4-oxo-butyl]carbamoylamino]-6-oxo-hexyl]amino]-2-oxo-1-(2-quinolylmethyl)ethyl]carbamoyl]-3-pyridyl]methylamino]-2-oxo-ethyl]-3-hydroxy-6-methyl-2-oxo-pyridine-4-carbonyl]amino]ethyl-[3-[2-[[1-[2-[[6-[[(1S)-2-[[(5S)-6-tert-butoxy-5-[[(1S)-4-tert-butoxy-1-tert-butoxycarbonyl-4-oxo-butyl]carbamoylamino]-6-oxo-hexyl]amino]-2-oxo-1-(2-quinolylmethyl)ethyl]carbamoyl]-3-pyridyl]methylamino]-2-oxo-ethyl]-3-hydroxy-6-methyl-2-oxo-pyridine-4-carbonyl]amino]ethyl-[2-[[1-(carboxymethyl)-3-hydroxy-6-methyl-2-oxo-pyridine-4-carbonyl]amino]ethyl]amino]propyl]amino]ethylcarbamoyl]-3-hydroxy-6-methyl-2-oxo-1-pyridyl]acetic acid

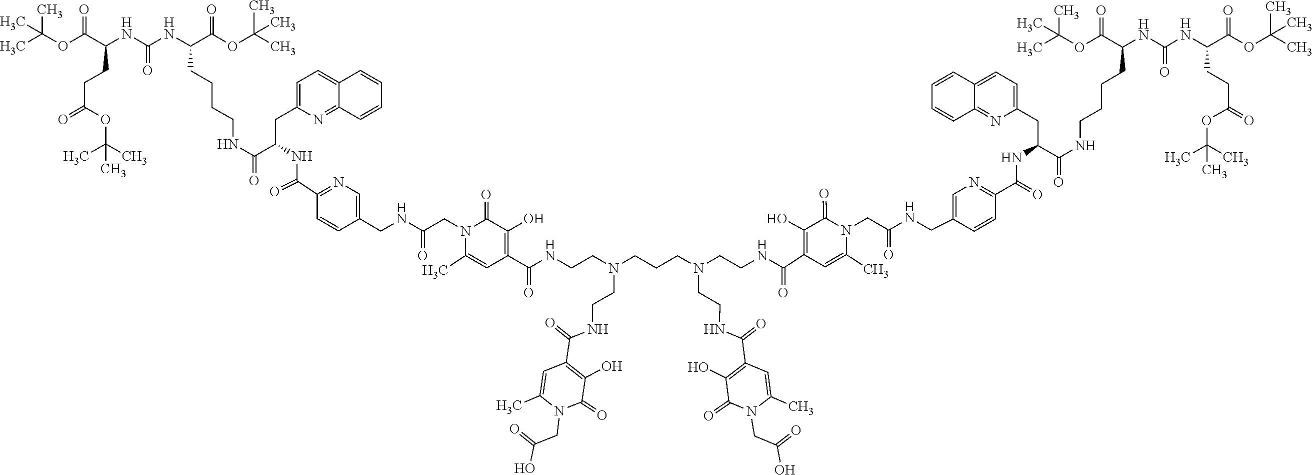 [SMILES: Cc1cc(C(=O)NCCN(CCCN(CCNC(=O)c2cc(C)n(CC(=O)O)c(=O)c2O)CCNC(=O)c2cc(C)n(CC(=O)NCc3ccc(C(=O)N[C@@H](Cc4ccc5ccccc5n4)C(=O)NCCCC[C@H](NC(=O)N[C@@H](CCC(=O)OC(C)(C)C)C(=O)OC(C)(C)C)C(=O)OC(C)(C)C)nc3)c(=O)c2O)CCNC(=O)c2cc(C)n(CC(=O)NCc3ccc(C(=O)N[C@@H](Cc4ccc5ccccc5n4)C(=O)NCCCC[C@H](NC(=O)N[C@@H](CCC(=O)OC(C)(C)C)C(=O)OC(C)(C)C)C(=O)OC(C)(C)C)nc3)c(=O)c2O)c(O)c(=O)n1CC(=O)O]

2,2′,2″,2′″-(propane-1,3-diylbis{nitrilobis[(ethane-2,1-diyl)carbamoyl(3-hydroxy-6-methyl-2-oxopyridine-4,1(2H)-diyl)]})tetraacetic acid (3.50 mg, 3.23 μmol) and tri-tert-butyl (3S,10S,14S)-1-[5-(aminomethyl)pyridin-2-yl]-1,4,12-trioxo-3-[(quinolin-2-yl)methyl]-2,5,11,13-tetraazahexadecane-10,14,16-tricarboxylate (2.65 mg, 3.23 μmol) are dissolved in NMP (610 μL). DIPEA (3.8 μL, 22 μmol) is added. PyAOP (2.1 mg, 4.04 μmol) in NMP 170 μL is added. Reaction mix is diluted with 20% ACN/water/0.1% TFA (8 mL) and the products purified by preparative HPLC (RP-HPLC using Äkta pure system, Column: Phenomenex Luna 5 μm C18(2) 100 Å, 250×21.2 mm, Mobile phase: Water/0.1% TFA; ACN Gradient: 20-70% B over 40 min Flow: 10 mL/min Detection: UV 280/335 nm, tR product: 35 min) afforded 1.5 mg (17% yield) of the target compound.

LC-MS (Method K, gradient: 10-70% B over 3 min): Rₜ=2.35 min; MS (ESIpos): m/z=1344.0 [M+2H]²⁺

Intermediate 154 Trimer

### 2-[4-[2-[3-[bis[2-[[1-[2-[[6-[[(1S)-2-[[(5S)-6-tert-butoxy-5-[[(1S)-4-tert-butoxy-1-tert- butoxycarbonyl-4-oxo-butyl]carbamoylamino]-6-oxo-hexyl]amino]-2-oxo-1-(2-quinolylmethyl)ethyl]carbamoyl]-3-pyridyl]methylamino]-2-oxo-ethyl]-3-hydroxy-6-methyl-2-oxo-pyridine-4-carbonyl]amino]ethyl]amino]propyl-[2-[[1-[2-[[6-[[(1S)-2-[[(5S)-6-tert-butoxy-5-[[(1S)-4-tert-butoxy-1-tert-butoxycarbonyl-4-oxo-butyl]carbamoylamino]-6-oxo-hexyl]amino]-2-oxo-1-(2-quinolylmethyl)ethyl]carbamoyl]-3-pyridyl]methylamino]-2-oxo-ethyl]-3-hydroxy-6-methyl-2-oxo-pyridine-4-carbonyl]amino]ethyl]amino]ethylcarbamoyl]-3-hydroxy-6-methyl-2-oxo-1-pyridyl]acetic acid

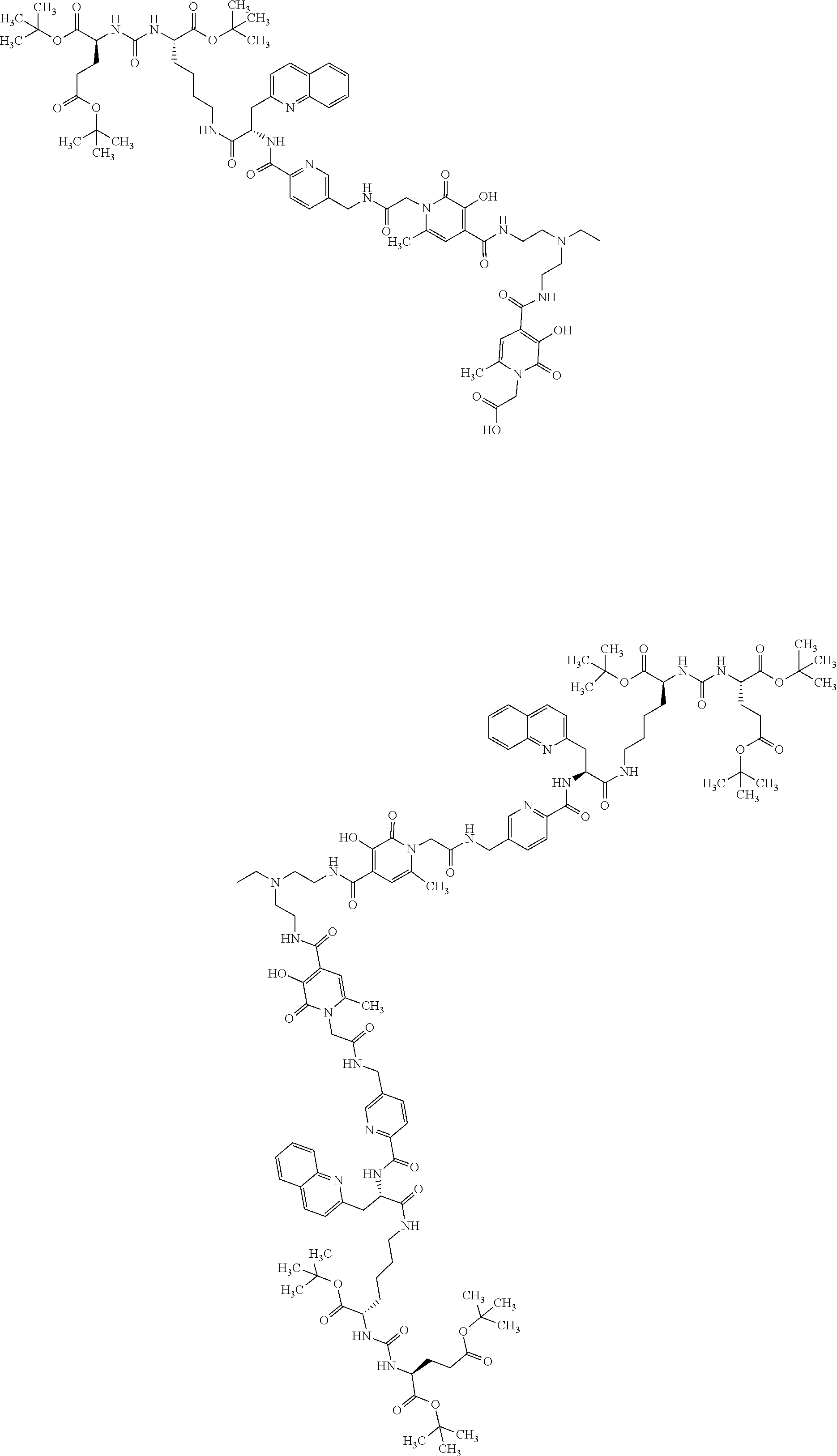

2,2′,2″,2′″-(propane-1,3-diylbis{nitrilobis[(ethane-2,1-diyl)carbamoyl(3-hydroxy-6-methyl-2-oxopyridine-4,1(2H)-diyl)]})tetraacetic acid (3.50 mg, 3.23 μmol) and tri-tert-butyl (3S,10S,14S)-1-[5-(aminomethyl)pyridin-2-yl]-1,4,12-trioxo-3-[(quinolin-2-yl)methyl]-2,5,11,13-tetraazahexadecane-10,14,16-tricarboxylate (2.65 mg, 3.23 μmol) are dissolved in NMP (610 μL). DIPEA (3.8 μL, 22 μmol) is added. PyAOP (2.1 mg, 4.04 μmol) in NMP 170 μL is added. Reaction mix is diluted with 20% ACN/water/0.1% TFA (8 mL) and the products purified by preparative HPLC (RP-HPLC using Äkta pure system, Column: Phenomenex Luna 5 μm C18(2) 100 Å, 250×21.2 mm, Mobile phase: Water/0.1% TFA; ACN Gradient: 20-70% B over 40 min Flow: 10 mL/min Detection: UV 280/335 nm, tR product: 40 min) afforded 1.0 mg (9% yield) of the target compound.

LC-MS (Method K, gradient: 10-70% B over 3 min): Rₜ=2.90 min; MS (ESIpos): m/z=1744.4 [M+2H]²⁺

Intermediate 155 Tetramer

### ditert-butyl (2S)-2-[[(1S)-5-[[(2S)-2-[[5-[[[2-[4-[2-[3-[bis[2-[[1-[2-[[6-[[(1S)-2-[[(5S)-6-tert-butoxy-5-[[(1S)-4-tert-butoxy-1-tert-butoxycarbonyl-4-oxo-butyl]carbamoylamino]-6-oxo-hexyl]amino]-2-oxo-1-(2-quinolylmethyl)ethyl]carbamoyl]-3-pyridyl]methylamino]-2-oxo-ethyl]-3-hydroxy-6-methyl-2-oxo-pyridine-4-carbonyl]amino]ethyl]amino]propyl-[2-[[1-[2-[[6-[[(1S)-2-[[(5S)-6-tert-butoxy-5-[[(1S)-4-tert-butoxy-1-tert-butoxycarbonyl-4-oxo-butyl]carbamoylamino]-6-oxo-hexyl]amino]-2-oxo-1-(2-quinolylmethyl)ethyl]carbamoyl]-3-pyridyl]methylamino]-2-oxo-ethyl]-3-hydroxy-6-methyl-2-oxo-pyridine-4-carbonyl]amino]ethyl]amino]ethylcarbamoyl]-3-hydroxy-6-methyl-2-oxo-1-pyridyl]acetyl]amino]methyl]pyridine-2-carbonyl]amino]-3-(2-quinolyl)propanoyl]amino]-1-tert-butoxycarbonyl-pentyl]carbamoylamino]pentanedioate

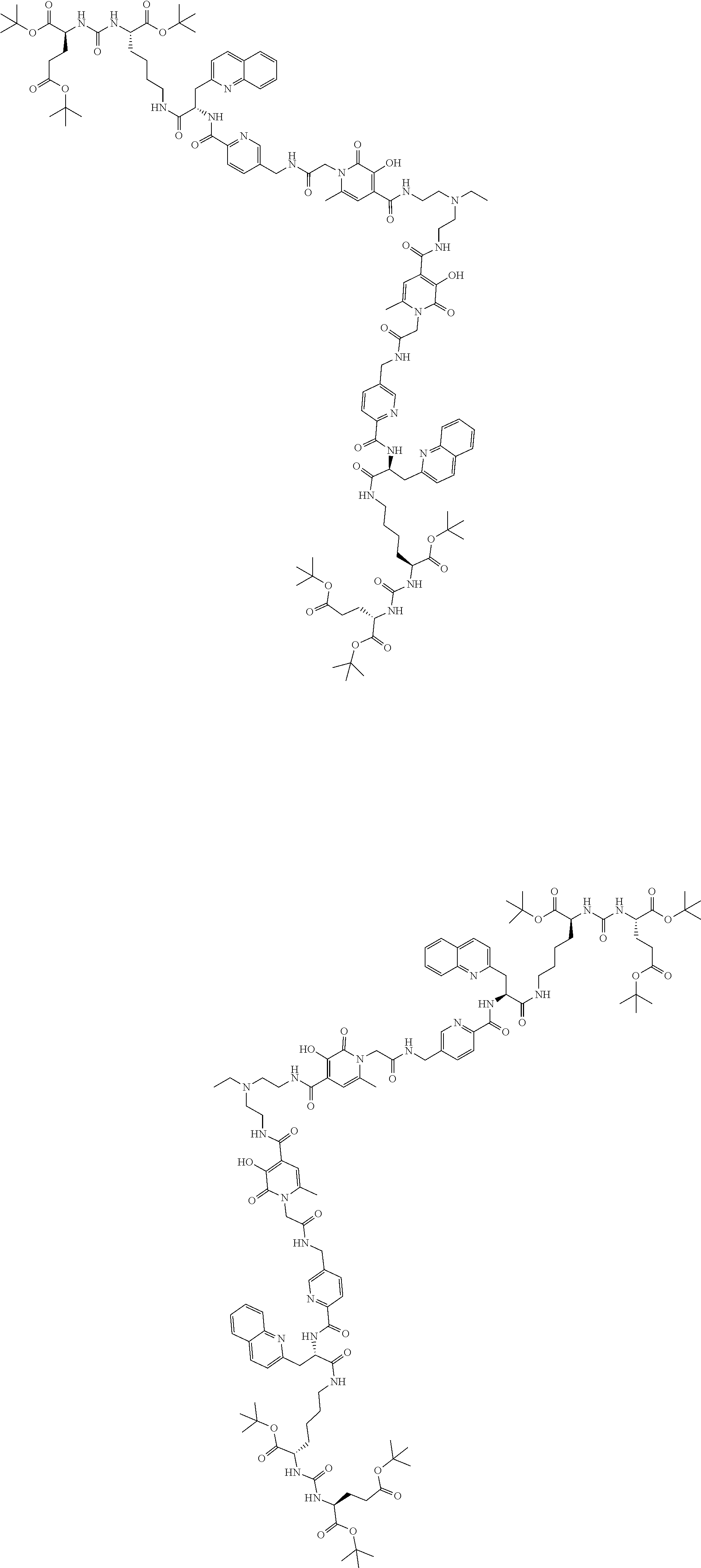

2,2′,2″,2′″-(propane-1,3-diylbis{nitrilobis[(ethane-2,1-diyl)carbamoyl(3-hydroxy-6-methyl-2-oxopyridine-4,1(2H)-diyl)]})tetraacetic acid (3.50 mg, 3.23 μmol) and tri-tert-butyl (3S,10S,14S)-1-[5-(aminomethyl)pyridin-2-yl]-1,4,12-trioxo-3-[(quinolin-2-yl)methyl]-2,5,11,13-tetraazahexadecane-10,14,16-tricarboxylate (2.65 mg, 3.23 μmol) are dissolved in NMP (610 μL). DIPEA (3.8 μL, 22 μmol) is added. PyAOP (2.1 mg, 4.04 μmol) in NMP 170 μL is added. Reaction mix is diluted with 20% ACN/water/0.1% TFA (8 mL) and the products purified by preparative HPLC (RP-HPLC using Äkta pure system, Column: Phenomenex Luna 5 μm C18(2) 100 Å, 250×21.2 mm, Mobile phase: Water/0.1% TFA; ACN Gradient: 20-70% B over 40 min Flow: 10 mL/min Detection: UV 280/335 nm, tR product: 43 min) afforded 1.1 mg (8% yield) of the target compound.

LC-MS (Method K, gradient: 10-70% B over 3 min): Rₜ=3.15 min; MS (ESIpos): m/z=1430.4 [M+3H]³⁺

### Example 34-C Monomer

### N⁶—{N-[5-({2-[4-({2-[{3-[bis(2-{[1-(carboxymethyl)-3-hydroxy-6-methyl-2-oxo-1,2-dihydropyridine-4-carbonyl]amino}ethyl)amino]propyl}(2-{[1-(carboxymethyl)-3-hydroxy-6-methyl-2-oxo-1,2-dihydropyridine-4-carbonyl]amino}ethyl)amino]ethyl}carbamoyl)-3-hydroxy-6-methyl-2-oxopyridin-1(2H)-yl]acetamido}methyl)pyridine-2-carbonyl]-3-(quinolin-2-yl)-L-alanyl}-N²-{[(1S)-1,3-dicarboxypropyl]carbamoyl}-L-lysine

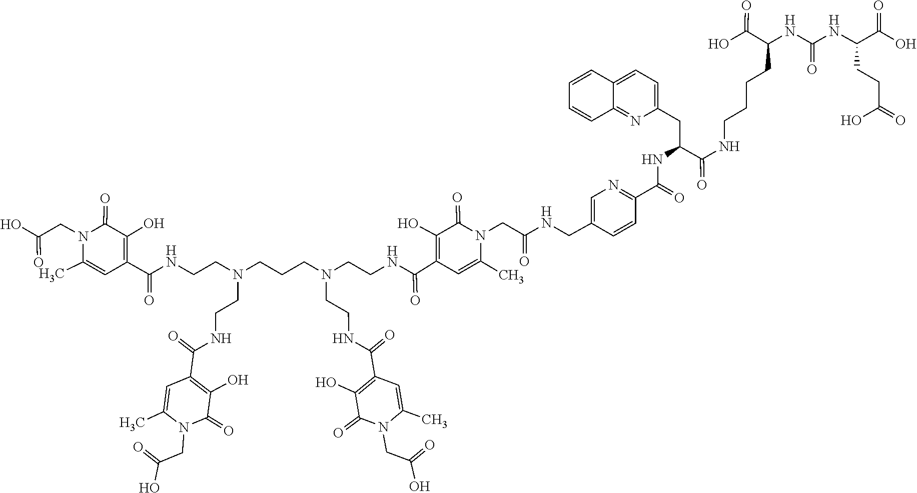

2,2′-{({3-[(2-{[1-(2-{[(6-{[(7S,11S,18S)-7,11-bis(tert-butoxycarbonyl)-2,2-dimethyl-4,9,17-trioxo-19-(quinolin-2-yl)-3-oxa-8,10,16-triazanonadecan-18-yl]carbamoyl}pyridin-3-yl)methyl]amino}-2-oxoethyl)-3-hydroxy-6-methyl-2-oxo-1,2-dihydropyridine-4-carbonyl]amino}ethyl)(2-{[1-(carboxymethyl)-3-hydroxy-6-methyl-2-oxopyridine-4(2H)-carbonyl]amino}ethyl)amino]propyl}azanediyl)bis[(ethane-2,1-diyl)carbamoyl(3-hydroxy-6-methyl-2-oxopyridine-4,1(2H)-diyl)]}diacetic acid (1.60 mg, 0.849 μmol) is treated with 90% TFA in water (0.5 mL). Water (18 mL) is added and solution lyophilised.affording 1.40 mg (95% purity, 91% yield) of the target compound.

LC-MS (Method K, gradient: 10-50% B over 3 min): Rₜ=1.47 min; MS (ESIpos): m/z=1716.7 [M+H]⁺

### Example 34-C Dimer

### (3S,10S,14S,3'S,10'S,14'S)-1,1′-(propane-1,3-diylbis{[(2-{[1-(carboxymethyl)-3-hydroxy-6-methyl-2-oxo-1,2-dihydropyridine-4-carbonyl]amino}ethyl)azanediyl]ethane-2,1-diylcarbamoyl(3-hydroxy-6-methyl-2-oxopyridine-4,1(2H)-diyl)(1-oxoethane-2,1-diyl)azanediylmethylenepyridine-5,2-diyl})bis{1,4,12-trioxo-3-[(quinolin-2-yl)methyl]-2,5,11,13-tetraazahexadecane-10,14,16-tricarboxylic acid}

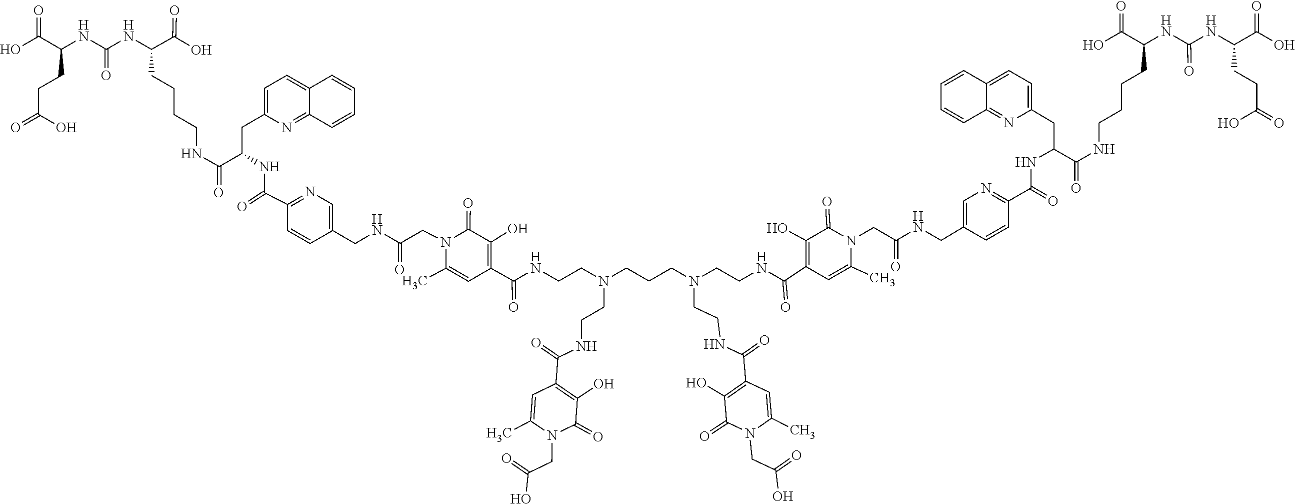 [SMILES: Cc1cc(C(=O)NCCN(CCCN(CCNC(=O)c2cc(C)n(CC(=O)O)c(=O)c2O)CCNC(=O)c2cc(C)n(CC(=O)NCc3ccc(C(=O)N[C@@H](Cc4ccc5ccccc5n4)C(=O)NCCCC[C@H](NC(=O)N[C@@H](CCC(=O)O)C(=O)O)C(=O)O)nc3)c(=O)c2O)CCNC(=O)c2cc(C)n(CC(=O)NCc3ccc(C(=O)NC(Cc4ccc5ccccc5n4)C(=O)NCCCC[C@H](NC(=O)N[C@@H](CCC(=O)O)C(=O)O)C(=O)O)nc3)c(=O)c2O)c(O)c(=O)n1CC(=O)O]

2-[4-[2-[2-[[1-[2-[[6-[[(1S)-2-[[(5S)-6-tert-butoxy-5-[[(1S)-4-tert-butoxy-1-tert-butoxycarbonyl-4-oxo-butyl]carbamoylamino]-6-oxo-hexyl]amino]-2-oxo-1-(2-quinolylmethyl)ethyl]carbamoyl]-3-pyridyl]methylamino]-2-oxo-ethyl]-3-hydroxy-6-methyl-2-oxo-pyridine-4-carbonyl]amino]ethyl-[3-[2-[[1-[2-[[6-[[(1S)-2-[[(5S)-6-tert-butoxy-5-[[(1S)-4-tert-butoxy-1-tert-butoxycarbonyl-4-oxo-butyl]carbamoylamino]-6-oxo-hexyl]amino]-2-oxo-1-(2-quinolylmethyl)ethyl]carbamoyl]-3-pyridyl]methylamino]-2-oxo-ethyl]-3-hydroxy-6-methyl-2-oxo-pyridine-4-carbonyl]amino]ethyl-[2-[[1-(carboxymethyl)-3-hydroxy-6-methyl-2-oxo-pyridine-4-carbonyl]amino]ethyl]amino]propyl]amino]ethylcarbamoyl]-3-hydroxy-6-methyl-2-oxo-1-pyridyl]acetic acid (1.50 mg, 0.558 μmol) is treated with 90% TFA in water (0.5 mL). Water (8 mL) is added and solution subjected to preparative HPLC (RP-HPLC using Äkta pure system, Column: Phenomenex Luna 5 μm C18(2) 100 Å, 250×21.2 mm Mobile phase: Water/0.1% TFA; ACN, Gradient: 10-50% B over 40 min Flow: 10 mL/min Detection: UV 280/335 nm, tR product: 24 min) affording 1.00 mg (95% purity, 72% yield) of the target compound.

LC-MS (Method K, gradient: 10-50% B over 3 min): Rₜ=1.65 min; MS (ESIpos): m/z=1175.5 [M+2H]²⁺

### Example 34-C Trimer

### (3S,10S,14S,3'S,10'S,14'S)-1,1′-{({3-[(2-{[1-(2-{[(6-{[(2S)-1-{[(5S)-5-carboxy-5-({[(1S)-1,3-dicarboxypropyl]carbamoyl}amino)pentyl]amino}-1-oxo-3-(quinolin-2-yl)propan-2-yl]carbamoyl}pyridin-3-yl)methyl]amino}-2-oxoethyl)-3-hydroxy-6-methyl-2-oxopyridine-4(2H)-carbonyl]amino}ethyl)(2-{[1-(carboxymethyl)-3-hydroxy-6-methyl-2-oxo-1,2-dihydropyridine-4-carbonyl]amino}ethyl)amino]propyl}azanediyl)bis[(ethane-2,1-diyl)carbamoyl(3-hydroxy-6-methyl-2-oxopyridine-4,1(2H)-diyl)(1-oxoethane-2,1-diyl)azanediylmethylenepyridine-5,2-diyl]}bis{1,4,12-trioxo-3-[(quinolin-2-yl)methyl]-2,5,11,13-tetraazahexadecane-10,14,16-tricarboxylic acid}

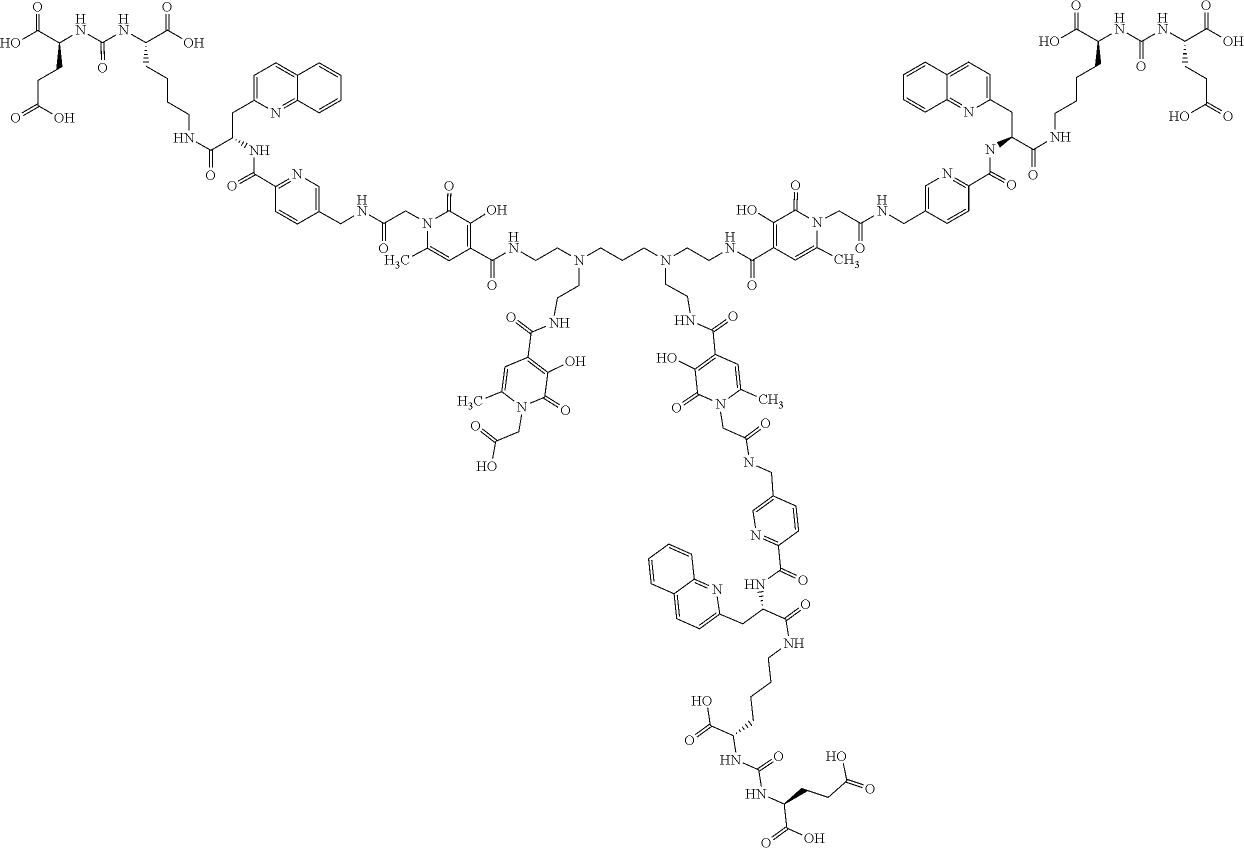

2-[4-[2-[3-[bis[2-[[1-[2-[[6-[[(1S)-2-[[(5S)-6-tert-butoxy-5-[[(1S)-4-tert-butoxy-1-tert- butoxycarbonyl-4-oxo-butyl]carbamoylamino]-6-oxo-hexyl]amino]-2-oxo-1-(2-quinolylmethyl)ethyl]carbamoyl]-3-pyridyl]methylamino]-2-oxo-ethyl]-3-hydroxy-6-methyl-2-oxo-pyridine-4-carbonyl]amino]ethyl]amino]propyl-[2-[[1-[2-[[6-[[(1S)-2-[[(5S)-6-tert-butoxy-5-[[(1S)-4-tert-butoxy-1-tert-butoxycarbonyl-4-oxo-butyl]carbamoylamino]-6-oxo-hexyl]amino]-2-oxo-1-(2-quinolylmethyl)ethyl]carbamoyl]-3-pyridyl]methylamino]-2-oxo-ethyl]-3-hydroxy-6-methyl-2-oxo-pyridine-4-carbonyl]amino]ethyl]amino]ethylcarbamoyl]-3-hydroxy-6-methyl-2-oxo-1-pyridyl]acetic acid (1.00 mg, 0.287 μmol) is treated with 90% TFA in water (0.5 mL). Water (8 mL) is added and solution subjected to preparative HPLC (Äkta pure system, Column: Phenomenex Luna 5 μm C18(2) 100 Å, 250×21.2 mm Mobile phase: Water/0.1% TFA; ACN Gradient: 10-50% B over 40 min Flow: 10 mL/min Detection: UV 280/335 nm, tR: 27 min affording 500 μg (95% purity, 56% yield) of the target compound.

LC-MS (Method K, gradient: 10-50% B over 3 min): Rₜ=1.78 min; MS (ESIpos): m/z=1492.4 [M+2H]²⁺

### Example 34-C Tetramer

### (2S)-2-[[(1S)-5-[[(2S)-2-[[5-[[[2-[4-[2-[3-[bis[2-[[1-[2-[[6-[[(1S)-2-[[(5S)-5-carboxy-5-[[(1S)-1,3-dicarboxypropyl]carbamoylamino]pentyl]amino]-2-oxo-1-(2-quinolylmethyl)ethyl]carbamoyl]-3-pyridyl]methylamino]-2-oxo-ethyl]-3-hydroxy-6-methyl-2-oxo-pyridine-4-carbonyl]amino]ethyl]amino]propyl-[2-[[1-[2-[[6-[[(1S)-2-[[(5S)-5-carboxy-5-[[(1S)-1,3-dicarboxypropyl]carbamoylamino]pentyl]amino]-2-oxo-1-(2-quinolylmethyl)ethyl]carbamoyl]-3-pyridyl]methylamino]-2-oxo-ethyl]-3-hydroxy-6-methyl-2-oxo-pyridine-4-carbonyl]amino]ethyl]amino]ethylcarbamoyl]-3-hydroxy-6-methyl-2-oxo-1-pyridyl]acetyl]amino]methyl]pyridine-2-carbonyl]amino]-3-(2-quinolyl)propanoyl]amino]-1-carboxy-pentyl]carbamoylamino]pentanedioic acid

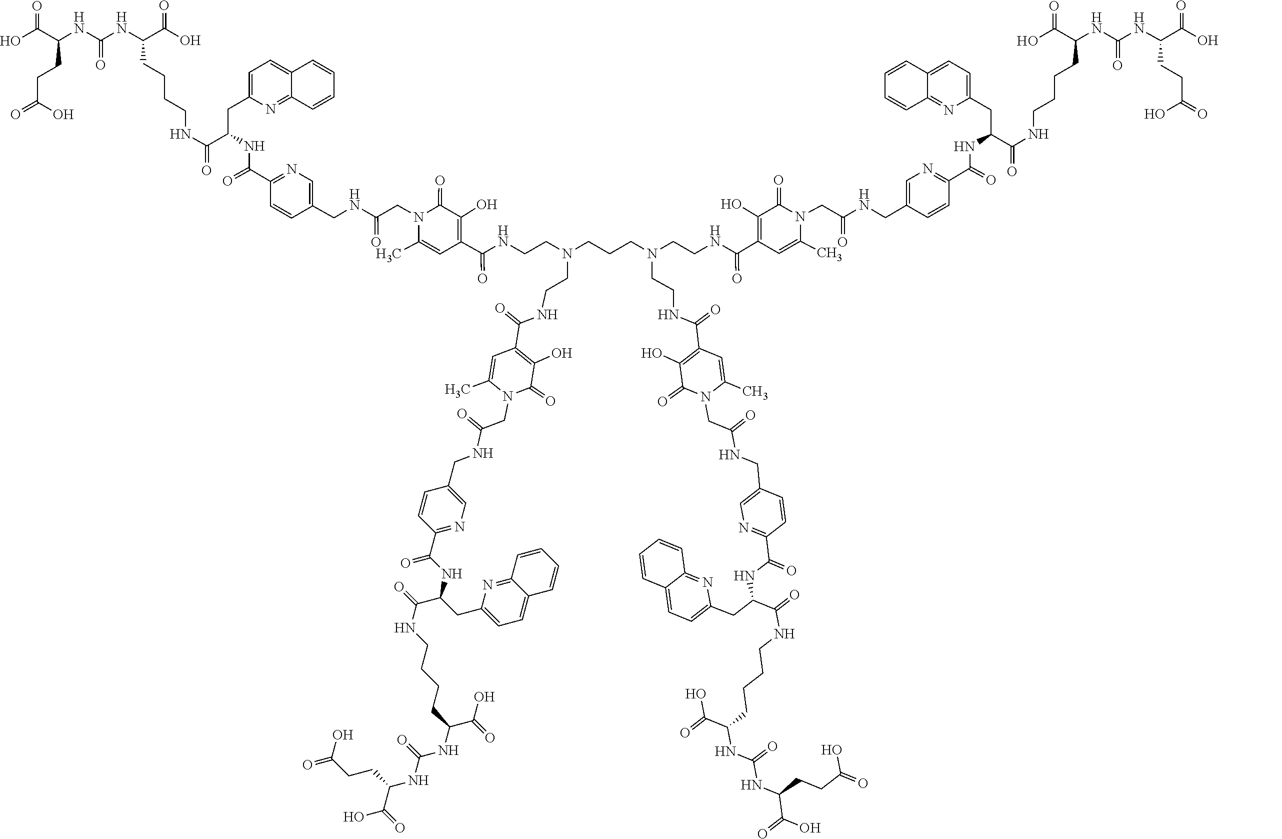

ditert-butyl (2S)-2-[[(1S)-5-[[(2S)-2-[[5-[[[2-[4-[2-[3-[bis[2-[[1-[2-[[6-[[(1S)-2-[[(5S)-6-tert-butoxy-5-[[(1S)-4-tert-butoxy-1-tert-butoxycarbonyl-4-oxo-butyl]carbamoylamino]-6-oxo-hexyl]amino]-2-oxo-1-(2-quinolylmethyl)ethyl]carbamoyl]-3-pyridyl]methylamino]-2-oxo-ethyl]-3-hydroxy-6-methyl-2-oxo-pyridine-4-carbonyl]amino]ethyl]amino]propyl-[2-[[1-[2-[[6-[[(1S)-2-[[(5S)-6-tert-butoxy-5-[[(1S)-4-tert-butoxy-1-tert-butoxycarbonyl-4-oxo-butyl]carbamoylamino]-6-oxo-hexyl]amino]-2-oxo-1-(2-quinolylmethyl)ethyl]carbamoyl]-3-pyridyl]methylamino]-2-oxo-ethyl]-3-hydroxy-6-methyl-2-oxo-pyridine-4-carbonyl]amino]ethyl]amino]ethylcarbamoyl]-3-hydroxy-6-methyl-2-oxo-1-pyridyl]acetyl]amino]methyl]pyridine-2-carbonyl]amino]-3-(2-quinolyl)propanoyl]amino]-1-tert-butoxycarbonyl-pentyl]carbamoylamino]pentanedioate (1.10 mg, 0.256 μmol) is treated with 90% TFA in water (0.5 mL). Water (18 mL) is added and solution affording 900 μg (95% purity, 92% yield) of the target compound

LC-MS (Method K, gradient: 10-50% B over 3 min): Rₜ=1.83 min; MS (ESIpos): m/z=1206.2 [M+3H]³⁺

Intermediate 156

### tri-tert-butyl (3S,10S,14S)-1,4,12-trioxo-3-[(quinolin-2-yl)methyl]-1-[5-({2-[4,7,10-tris(2-tert-butoxy-2-oxoethyl)-1,4,7,10-tetraazacyclododecan-1-yl]acetamido}methyl)pyridin-2-yl]-2,5,11,13-tetraazahexadecane-10,14,16-tricarboxylate

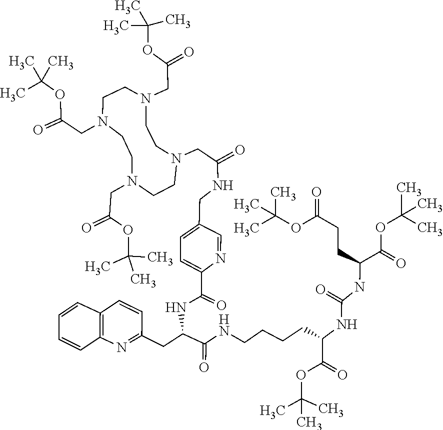

[4,7,10-tris(2-tert-butoxy-2-oxoethyl)-1,4,7,10-tetraazacyclododecan-1-yl]acetic acid (64.3 mg, 112 μmol; CAS-RN:[137076-54-1]), [(1H-benzotriazol-1-yl)oxy](dimethylamino)-N,N-dimethylmethaniminium hexafluoridophosphate(1−) (42.0 mg, 111 μmol; CAS-RN:94790-37-1) and N,N-diisopropylethylamine (14 μl, 84 μmol) were stirred in DMF (1.1 ml) for 10 min at rt. tri-tert-butyl (10S,14S)-1-[5-(aminomethyl)pyridin-2-yl]-1,4,12-trioxo-3-[(quinolin-2-yl)methyl]-2,5,11,13-tetraazahexadecane-10,14,16-tricarboxylate (23.0 mg, 28.0 μmol) was added and the mixture was stirred overnight at rt. The mixture was evaporated and purified by preparative HPLC (C18, acetonitrile/water with 0.1% formic acid) to give 20.0 mg (80% purity, 41% yield) of the target compound.

¹H-NMR (400 MHz, DMSO-d6) δ[ppm]: 1.232 (0.81), 1.324 (3.29), 1.366 (9.69), 1.375 (12.53), 1.379 (16.00), 1.395 (7.35), 1.401 (4.60), 1.439 (2.25), 2.336 (0.86), 2.518 (9.92), 2.522 (6.40), 2.539 (0.68), 2.660 (0.86), 2.687 (0.50), 3.558 (0.59), 7.905 (0.59), 7.925 (0.63), 8.588 (0.45).

### Example 34-B

### N⁶-{3-(quinolin-2-yl)-N-[5-({2-[4,7,10-tris(carboxymethyl)-1,4,7,10-tetraazacyclododecan-1-yl]acetamido}methyl)pyridine-2-carbonyl]-L-alanyl}-N²-{[(1S)-1,3-dicarboxypropyl]carbamoyl}-L-lysine

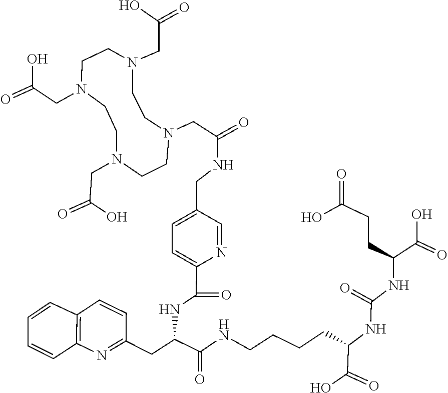 [SMILES: O=C(O)CC[C@H](NC(=O)N[C@@H](CCCCNC(=O)[C@H](Cc1ccc2ccccc2n1)NC(=O)c1ccc(CNC(=O)CN2CCN(CC(=O)O)CCN(CC(=O)O)CCN(CC(=O)O)CC2)cn1)C(=O)O)C(=O)O]

tri-tert-butyl (3S,10S,14S)-1,4,12-trioxo-3-[(quinolin-2-yl)methyl]-1-[5-({2-[4,7,10-tris(2-tert-butoxy-2-oxoethyl)-1,4,7,10-tetraazacyclododecan-1-yl]acetamido}methyl)pyridin-2-yl]-2,5,11,13-tetraazahexadecane-10,14,16-tricarboxylate (18.0 mg, 13.1 μmol) was solubilised in DCM (420 μl), TFA (200 μl, 2.6 mmol) was added and the mixture was stirred under argon overnight at rt. The mixture was evaporated and purified by preparative HPLC (C18, acetonitrile/water with 0.1% formic acid) to give 5.50 mg (95% purity, 38% yield) of the target compound.

¹H-NMR (400 MHz, DMSO-d6) δ[ppm]: 1.196 (0.54), 1.231 (1.42), 1.290 (0.61), 1.382 (3.44), 1.386 (2.97), 1.907 (0.47), 2.228 (0.74), 2.327 (3.98), 2.331 (2.97), 2.336 (1.35), 2.518 (16.00), 2.523 (10.26), 2.539 (1.62), 2.586 (0.68), 2.669 (4.12), 2.673 (2.97), 2.678 (1.35), 2.917 (0.88), 3.012 (1.89), 3.096 (0.81), 3.397 (2.03), 3.456 (1.08), 3.875 (0.61), 4.394 (0.68), 6.293 (0.61), 7.407 (1.01), 7.427 (0.95), 7.540 (0.74), 7.560 (0.47), 7.714 (0.41), 7.718 (0.41), 7.735 (0.68), 7.871 (0.47), 7.894 (0.74), 7.913 (0.68), 7.966 (0.95), 7.988 (0.74), 8.110 (0.41), 8.229 (0.95), 8.249 (0.88), 8.656 (0.47), 9.164 (0.47), 9.185 (0.47).

### Example 34-C Monomer 227Th

### [N6-{N-[5-({2-[4-({2-[{3-[bis(2-{[1-(carboxymethyl)-3-(hydroxy-kappaO)-6-methyl-2-oxo-1,2-dihydropyridine-4-carbonyl]amino}ethyl)amino]propyl}(2-{[1-(carboxymethyl)-3-(hydroxy-kappaO)-6-methyl-2-oxo-1,2-dihydropyridine-4-carbonyl]amino}ethyl)amino]ethyl}carbamoyl)-3-(hydroxy-kappaO)-6-methyl-2-oxopyridin-1(2H)-yl]acetamido}methyl)pyridine-2-carbonyl]-3-(quinolin-2-yl)-L-alanyl}-N2-{[(1S)-1,3-dicarboxypropyl]carbamoyl}-L-lysinato(4−)](227Th)Thorium

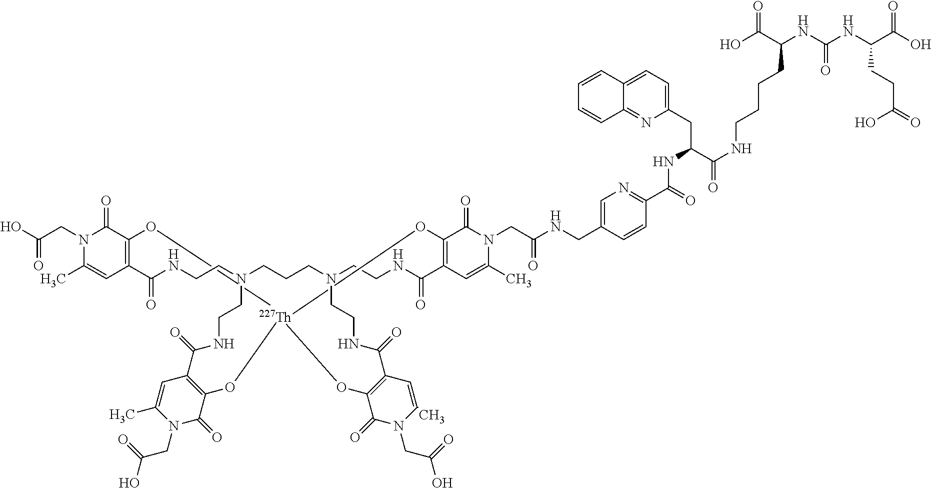

N6-{N-[5-({2-[4-({2-[{3-[bis(2-{[1-(carboxymethyl)-3-hydroxy-6-methyl-2-oxo-1,2-dihydropyridine-4-carbonyl]amino}ethyl)amino]propyl}(2-{[1-(carboxymethyl)-3-hydroxy-6-methyl-2-oxo-1,2-dihydropyridine-4-carbonyl]amino}ethyl)amino]ethyl}carbamoyl)-3-hydroxy-6-methyl-2-oxopyridin-1(2H)-yl]acetamido}methyl)pyridine-2-carbonyl]-3-(quinolin-2-yl)-L-alanyl}-N2-{[(1S)-1,3-dicarboxypropyl]carbamoyl}-L-lysine (2.0 μg) dissolved in 84 μL 30 mM citrate buffer (pH 5.5) containing 0.5 mg/mL pABA was mixed with thorium-227 in 0.5 M HCl (2 μL) at 0.3 MBq/nmol specific activity and RAC of 4.6 MBq/mL. 1M carbonate buffer pH 9 (8 μL) was added and mixture incubated for 90 min. The labelling efficiency was determined to be 97% by iTLC.

### Example 34-C Dimer ²²⁷Th

### (3S,10S,14S,3'S,10'S,14'S)-1,1′-(propane-1,3-diylbis{[(2-{[1-(carboxymethyl)-3-(hydroxy-kappaO)-6-methyl-2-oxo-1,2-dihydropyridine-4-carbonyl]amino}ethyl)azanediyl]ethane-2,1-diylcarbamoyl[3-(hydroxy-kappaO)-6-methyl-2-oxopyridine-4,1(2H)-diyl](1-oxoethane-2,1-diyl)azanediylmethylenepyridine-5,2-diyl})bis{1,4,12-trioxo-3-[(quinolin-2-yl)methyl]-2,5,11,13-tetraazahexadecane-10,14,16-tricarboxylato}(4−)(²²⁷Th)Thorium

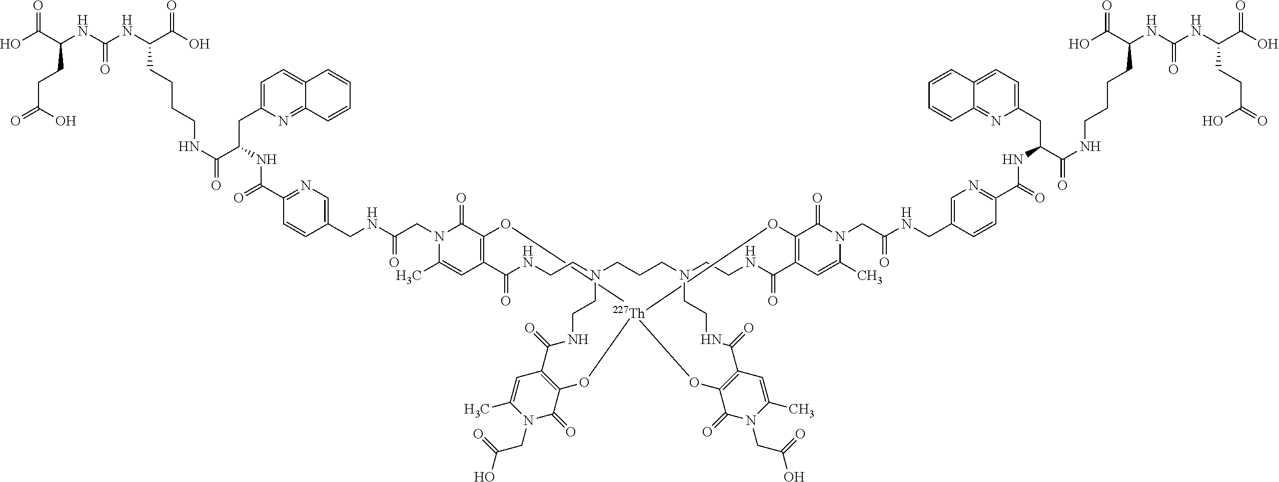

(3S,10S,14S,3'S,10'S,14'S)-1,1′-(propane-1,3-diylbis{[(2-{[1-(carboxymethyl)-3-hydroxy-6-methyl-2-oxo-1,2-dihydropyridine-4-carbonyl]amino}ethyl)azanediyl]ethane-2,1-diylcarbamoyl(3-hydroxy-6-methyl-2-oxopyridine-4,1(2H)-diyl)(1-oxoethane-2,1-diyl)azanediylmethylenepyridine-5,2-diyl})bis{1,4,12-trioxo-3-[(quinolin-2-yl)methyl]-2,5,11,13-tetraazahexadecane-10,14,16-tricarboxylic acid} (2.0 μg) dissolved in 63 μL 30 mM citrate buffer (pH 5.5) containing 0.5 mg/mL pABA was mixed with thorium-227 in 0.5 M HCl (2 μL) at 0.3 MBq/nmol specific activity and RAC of 4.5 MBq/mL. 1M carbonate buffer pH 9 (6 μL) was added and mixture incubated for 90 min. The labelling efficiency was determined to be 94% by iTLC.

### Example 34-C Trimer ²²⁷Th

### (3S,10S,14S,3'S,10'S,14'S)-1,1′-{({3-[(2-{[1-(2-{[(6-{[(2S)-1-{[(5S)-5-carboxy-5-({[(1S)-1,3-dicarboxypropyl]carbamoyl}amino)pentyl]amino}-1-oxo-3-(quinolin-2-yl)propan-2-yl]carbamoyl}pyridin-3-yl)methyl]amino}-2-oxoethyl)-3-(hydroxy-kappaO)-6-methyl-2-oxopyridine-4(2H)-carbonyl]amino}ethyl)(2-{[1-(carboxymethyl)-3-(hydroxy-kappaO)-6-methyl-2-oxo-1,2-dihydropyridine-4-carbonyl]amino}ethyl)amino]propyl}azanediyl)bis[(ethane-2,1-diyl)carbamoyl[3-(hydroxy-kappaO)-6-methyl-2-oxopyridine-4,1(2H)-diyl](1-oxoethane-2,1-diyl)azanediylmethylenepyridine-5,2-diyl]}bis{1,4,12-trioxo-3-[(quinolin-2-yl)methyl]-2,5,11,13-tetraazahexadecane-10,14,16-tricarboxylato}(4−)(²²⁷Th)Thorium

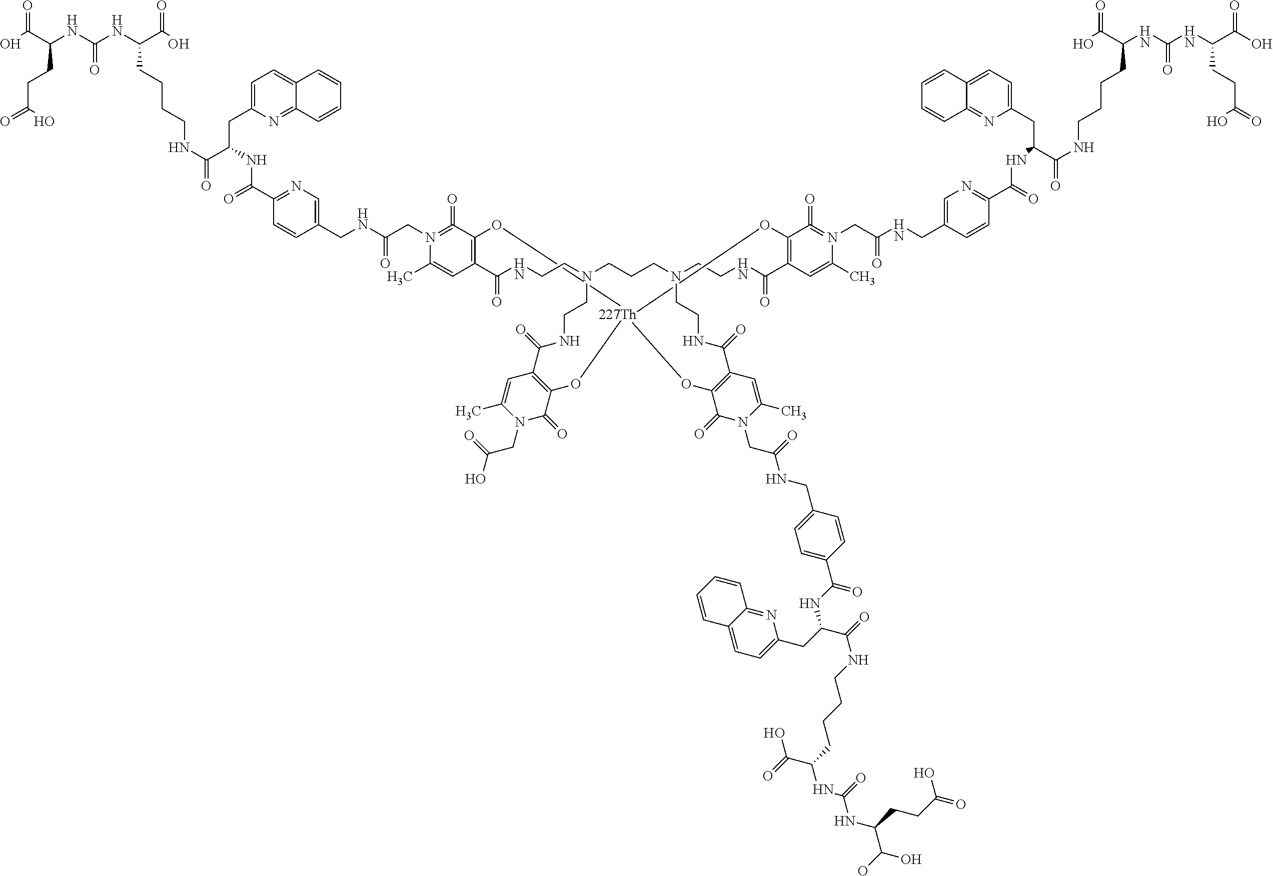

(3S,10S,14S,3'S,10'S,14'S)-1,1′-{({3-[(2-{[1-(2-{[(6-{[(2S)-1-{[(5S)-5-carboxy-5-({[(1S)-1,3-dicarboxypropyl]carbamoyl}amino)pentyl]amino}-1-oxo-3-(quinolin-2-yl)propan-2-yl]carbamoyl}pyridin-3-yl)methyl]amino}-2-oxoethyl)-3-hydroxy-6-methyl-2-oxopyridine-4(2H)-carbonyl]amino}ethyl)(2-{[1-(carboxymethyl)-3-hydroxy-6-methyl-2-oxo-1,2-dihydropyridine-4-carbonyl]amino}ethyl)amino]propyl}azanediyl)bis[(ethane-2,1-diyl)carbamoyl(3-hydroxy-6-methyl-2-oxopyridine-4,1(2H)-diyl)(1-oxoethane-2,1-diyl)azanediylmethylenepyridine-5,2-diyl]}bis{1,4,12-trioxo-3-[(quinolin-2-yl)methyl]-2,5,11,13-tetraazahexadecane-10,14,16-tricarboxylic acid} (4.0 μg) dissolved in 84 μL 30 mM citrate buffer (pH 5.5) containing 0.5 mg/mL pABA was mixed with thorium-227 in 0.5 M HCl (2 μL) at 0.3 MBq/nmol specific activity and RAC of 4.5 MBq/mL. 1M carbonate buffer pH 9 (8 μL) was added and mixture incubated for 90 min. The labelling efficiency was determined to be 99% by iTLC.

### Example 34-C Tetramer ²²⁷Th

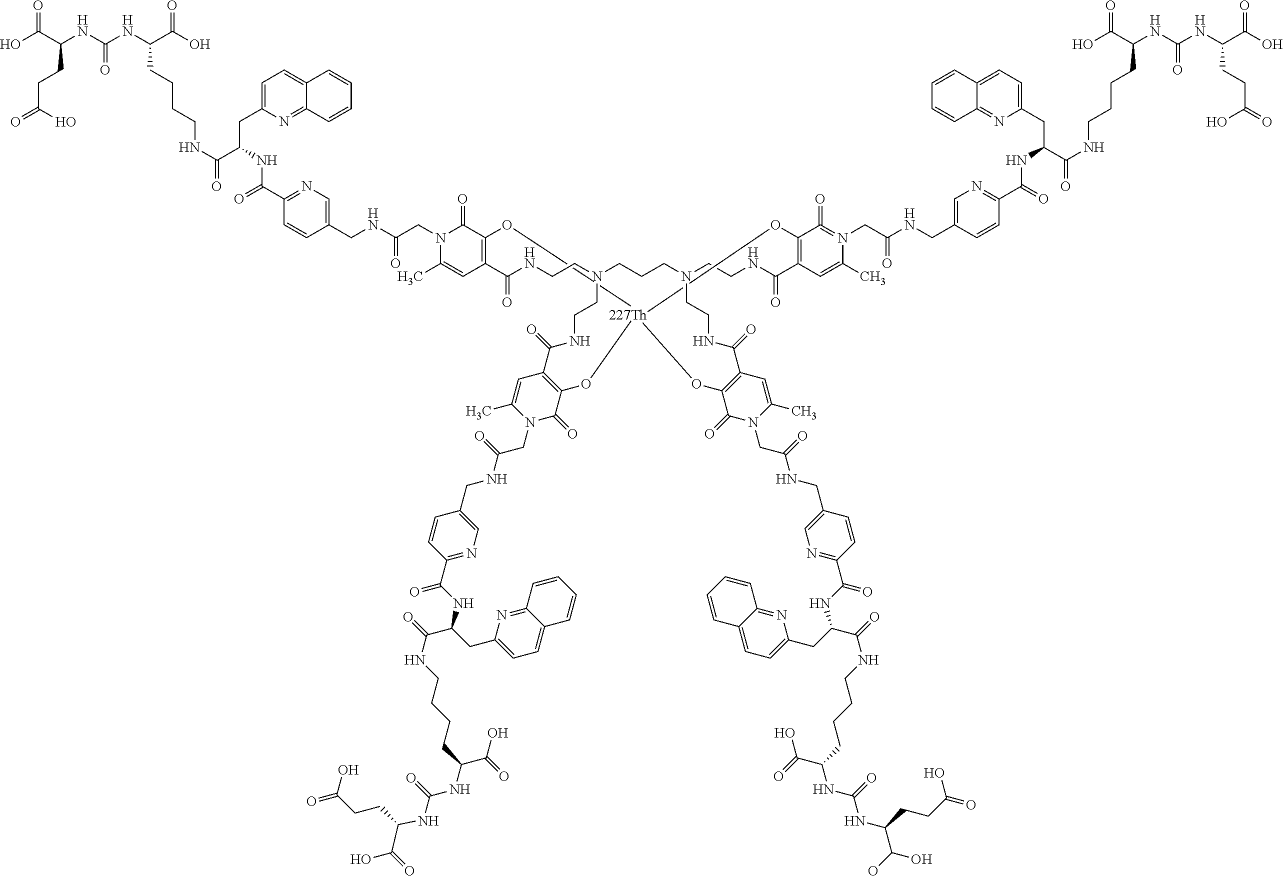

(2S)-2-[[(1 S)-5-[[(2S)-2-[[5-[[[2-[4-[2-[3-[bis[2-[[1-[2-[[6-[[(1 S)-2-[[(5S)-5-carboxy-5-[[(1 S)-1,3-dicarboxypropyl]carbamoylamino]pentyl]amino]-2-oxo-1-(2-quinolylmethyl)ethyl]carbamoyl]-3-pyridyl]methyl amino]-2-oxo-ethyl]-3-hydroxy-6-methyl-2-oxo-pyridine-4-carbonyl]amino]ethyl]amino]propyl-[2-[[1-[2-[[6-[[(1 S)-2-[[(5S)-5-carboxy-5-[[(1 S)-1,3-dicarboxypropyl]carbamoylamino]pentyl]amino]-2-oxo-1-(2-quinolylmethyl)ethyl]carbamoyl]-3-pyridyl]methylamino]-2-oxo-ethyl]-3-hydroxy-6-methyl-2-oxo-pyridine-4-carbonyl]amino]ethyl]amino]ethylcarbamoyl]-3-hydroxy-6-methyl-2-oxo-1-pyridyl]acetyl]amino]methyl]pyridine-2-carbonyl]amino]-3-(2-quinolyl)propanoyl]amino]-1-carboxy-pentyl]carbamoylamino]pentanedioic acid (4.0 μg) dissolved in 69 μL 30 mM citrate buffer (pH 5.5) containing 0.5 mg/mL pABA was mixed with thorium-227 in 0.5 M HCl (2 μL) at 0.3 MBq/nmol specific activity and RAC of 4.5 MBq/mL. 1M carbonate buffer pH 9 (7 μL) was added and mixture incubated for 4 hrs and 30 min. The labelling efficiency was determined to be 95% by iTLC.

### Example 34-B ²²⁷Th

### {N⁶-[3-(quinolin-2-yl)-N-{5-[(2-{4,7,10-tris[(carboxy-kappaO)methyl]-1,4,7,10-tetraazacyclododecan-1-yl-kappa⁴N¹,N⁴,N⁷,N¹⁰}acetamido)methyl]pyridine-2-carbonyl}-L-alanyl]-N²-{[(1S)-1,3-dicarboxypropyl]carbamoyl}-L-lysinato(4−)}(²²⁷Th)Thorate(1−)

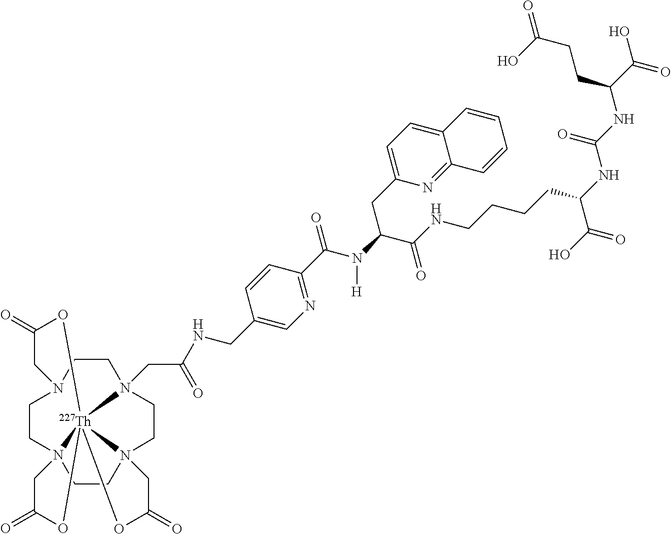

N6-{3-(quinolin-2-yl)-N-[5-({2-[4,7,10-tris(carboxymethyl)-1,4,7,10-tetraazacyclododecan-1-yl]acetamido}methyl)pyridine-2-carbonyl]-L-alanyl}-N²-{[(1S)-1,3-dicarboxypropyl]carbamoyl}-L-lysine (1.00 mg, 0.963 μmol) was dissolved in 400 mM sodium acetate buffer (pH 5) containing 0.5 mg/mL pABA. Thorium-227 in 400 mM sodium acetate buffer (pH 5) plus 20% EtOH was added giving a specific activity of 0.375 MBq/nmol and a RAC of 2.9 MBq/mL. The mixture was incubated at 95° C. for 60 min. The labelling efficiency was determined to be 79.6% by iTLC.

Intermediate 78 D

### tri-tert-butyl (5S,12S,16R)-1-(9H-fluoren-9-yl)-3,6,14-trioxo-5-[(quinolin-2-yl)methyl]-2-oxa-4,7,13,15-tetraazaoctadecane-12,16,18-tricarboxylate

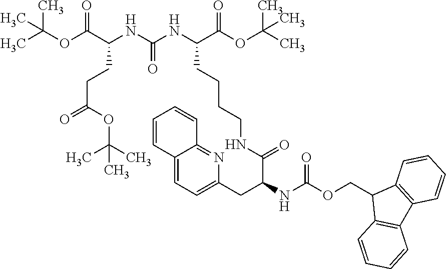 [SMILES: CC(C)(C)OC(=O)CC[C@@H](NC(=O)N[C@@H](CCCCNC(=O)[C@H](Cc1ccc2ccccc2n1)NC(=O)OCC1c2ccccc2-c2ccccc21)C(=O)OC(C)(C)C)C(=O)OC(C)(C)C]

di-tert-Butyl (2S)-2-({[(2S)-6-{[(2S)-2-amino-3-(quinolin-2-yl)propanoyl]amino}-1-tert-butoxy-1-oxohexan-2-yl]carbamoyl}amino)pentanedioate (183 mg, 70% purity, 262 μmol) and N-{[(9H-fluoren-9-yl)methoxy]carbonyl}-3-quinolin-2-yl-L-alanine (115 mg, 262 μmol) were solubilised in DMF (2.0 ml) at 0° C., then N-methylmorpholine (87 μl, 790 μmol) and HATU (150 mg, 393 μmol) were added and the mixture was stirred under argon at 0° C. for 45 min. The mixture was diluted with DCM and water, the organic phase was filtered through a phase separator and concentrated under reduced pressure. The crude material was purified by preparative HPLC (C18, acetonitrile/water with 0.1% formic acid) to give 83.0 mg (34% yield) of the target compound.

LC-MS (Method 1): Rₜ=1.58 min; MS (ESIpos): m/z=909 [M+H}⁺.

Intermediate 79 D

### di-tert-butyl (2R)-2-({[(2S)-6-{[(2S)-2-amino-3-(quinolin-2-yl)propanoyl]amino}-1-tert-butoxy-1-oxohexan-2-yl]carbamoyl}amino)pentanedioate

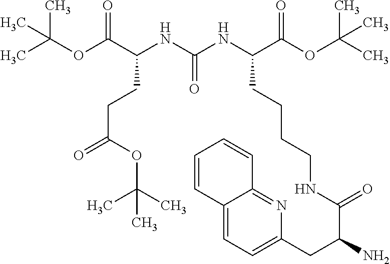 [SMILES: CC(C)(C)OC(=O)CC[C@@H](NC(=O)N[C@@H](CCCCNC(=O)[C@@H](N)Cc1ccc2ccccc2n1)C(=O)OC(C)(C)C)C(=O)OC(C)(C)C]

A mixture of tri-tert-butyl (5S,12S,16R)-1-(9H-fluoren-9-yl)-3,6,14-trioxo-5-[(quinolin-2-yl)methyl]-2-oxa-4,7,13,15-tetraazaoctadecane-12,16,18-tricarboxylate (100 mg, 110 μmol), piperidine (110 μl, 1.1 mmol; CAS-RN:[110-89-4]), and DMF (1.7 mL) was stirred at r.t. for 2 h. After that the mixture was concentrated under reduced pressure and the crude product was purified by preparative HPLC (C18, acetonitrile/0.1% ammonium hydroxide gradient) to give the title compound (48 mg, 64% yield).

LC-MS (Method 1): Rₜ=1.34 min; MS (ESIpos): m/z=689 [M+H}⁺.

Intermediate 156 D

### tri-tert-butyl (3S,10S,14R)-1-{5-[({[(9H-fluoren-9-yl)methoxy]carbonyl}amino)methyl]pyridin-2-yl}-1,4,12-trioxo-3-[(quinolin-2-yl)methyl]-2,5,11,13-tetraazahexadecane-10,14,16-tricarboxylate

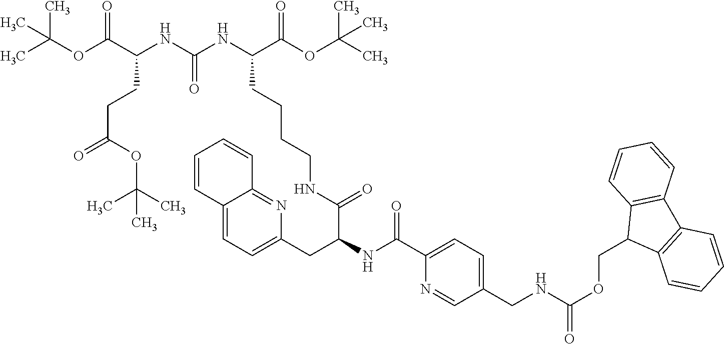 [SMILES: CC(C)(C)OC(=O)CC[C@@H](NC(=O)N[C@@H](CCCCNC(=O)[C@H](Cc1ccc2ccccc2n1)NC(=O)c1ccc(CNC(=O)OCC2c3ccccc3-c3ccccc32)cn1)C(=O)OC(C)(C)C)C(=O)OC(C)(C)C]

To a solution of di-tert-butyl (2R)-2-({[(2S)-6-{[(2S)-2-amino-3-(quinolin-2-yl)propanoyl]amino}-1-tert-butoxy-1-oxohexan-2-yl]carbamoyl}amino)pentanedioate (48.0 mg, 70.0 μmol) in DMF (1.1 mL) was added at r.t. 5-[({[(9H-fluoren-9-yl)methoxy]carbonyl}amino)methyl]pyridine-2-carboxylic acid (39.3 mg, 105 μmol), HATU (47.9 mg, 126 μmol; CAS-RN:[148893-10-1]), and 4-methylmorpholine (23 μl, 210 μmol; CAS-RN:[109-02-4]). After stirring for 1 h the mixture was diluted with DCM and water, the organic phase was washed with brine and filtered through a phase separator. The filtrate was concentrated under reduced pressure and purified by preparative HPLC (C18, acetonitrile/0.1% formic acid gradient) to give the title compound (30.0 mg, 41% yield).

LC-MS (Method 1): Rₜ=1.56 min; MS (ESIpos): m/z=1043 [M+H}+.

¹H-NMR (400 MHz, DMSO-d6) δ[ppm]: 1.360 (16.00), 1.387 (9.10), 2.323 (0.75), 2.327 (1.04), 2.331 (0.75), 2.518 (3.26), 2.523 (2.41), 2.665 (0.77), 2.669 (1.06), 2.673 (0.73), 4.363 (0.53), 4.380 (0.44), 7.308 (0.50), 7.403 (0.80), 7.424 (0.48), 7.664 (0.50), 7.685 (0.53), 7.874 (0.53), 7.894 (0.84), 7.916 (0.41), 7.943 (0.41), 8.225 (0.43).

Intermediate 157 D

### tri-tert-butyl (3S,10S,14R)-1-[5-(aminomethyl)pyridin-2-yl]-1,4,12-trioxo-3-[(quinolin-2-yl)methyl]-2,5,11,13-tetraazahexadecane-10,14,16-tricarboxylate

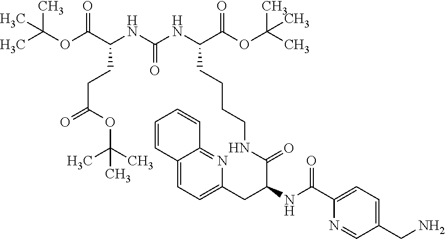 [SMILES: CC(C)(C)OC(=O)CC[C@@H](NC(=O)N[C@@H](CCCCNC(=O)[C@H](Cc1ccc2ccccc2n1)NC(=O)c1ccc(CN)cn1)C(=O)OC(C)(C)C)C(=O)OC(C)(C)C]

A mixture of tri-tert-butyl (3S,10S,14R)-1-{5-[({[(9H-fluoren-9-yl)methoxy]carbonyl}amino)methyl]pyridin-2-yl}-1,4,12-trioxo-3-[(quinolin-2-yl)methyl]-2,5,11,13-tetraazahexadecane-10,14,16-tricarboxylate (30.0 mg, 28.8 μmol), piperidine (57 μl, 580 μmol; CAS-RN:[110-89-4]) and DMF was stirred at r.t. for 2 h. After that the mixture was concentrated under reduced pressure and purified by preparative HPLC (C18, acetonitrile, 0.1% ammonium hydroxide gradient) to give 20.0 mg of the title compound (85% yield).

LC-MS (Method 2): Rₜ=1.33 min; MS (ESIpos): m/z=821 [M+H]+

¹H-NMR (400 MHz, DMSO-d6) δ[ppm]: −0.026 (5.62), 0.852 (1.07), 1.232 (10.46), 1.366 (16.00), 1.391 (8.80), 2.323 (1.43), 2.327 (2.01), 2.331 (1.43), 2.336 (0.64), 2.518 (6.40), 2.523 (4.72), 2.665 (1.46), 2.669 (2.02), 2.673 (1.41), 2.678 (0.61), 3.387 (0.47), 3.804 (1.19), 5.229 (0.96), 7.401 (0.56), 7.422 (0.54), 7.892 (1.39), 7.896 (1.44), 8.227 (0.42), 8.605 (0.53).

Intermediate 157 D Monomer

### 2,2′-{({3-[(2-{[1-(2-{[(6-{[(7R,11S,18S)-7,11-bis(tert-butoxycarbonyl)-2,2-dimethyl-4,9,17-trioxo-19-(quinolin-2-yl)-3-oxa-8,10,16-triazanonadecan-18-yl]carbamoyl}pyridin-3-yl)methyl]amino}-2-oxoethyl)-3-hydroxy-6-methyl-2-oxo-1,2-dihydropyridine-4-carbonyl]amino}ethyl)(2-{[1-(carboxymethyl)-3-hydroxy-6-methyl-2-oxopyridine-4(2H)-carbonyl]amino}ethyl)amino]propyl}azanediyl)bis[(ethane-2,1-diyl)carbamoyl(3-hydroxy-6-methyl-2-oxopyridine-4,1(2H)-diyl)]}diacetic acid

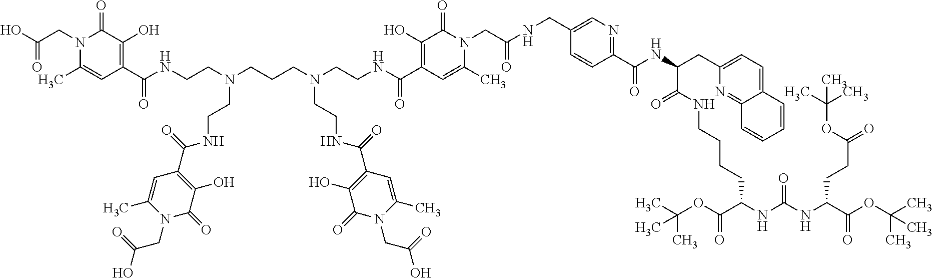 [SMILES: Cc1cc(C(=O)NCCN(CCCN(CCNC(=O)c2cc(C)n(CC(=O)O)c(=O)c2O)CCNC(=O)c2cc(C)n(CC(=O)NCc3ccc(C(=O)N[C@@H](Cc4ccc5ccccc5n4)C(=O)NCCCC[C@H](NC(=O)N[C@H](CCC(=O)OC(C)(C)C)C(=O)OC(C)(C)C)C(=O)OC(C)(C)C)nc3)c(=O)c2O)CCNC(=O)c2cc(C)n(CC(=O)O)c(=O)c2O)c(O)c(=O)n1CC(=O)O]

2,2′,2″,2′″-(propane-1,3-diylbis{nitrilobis[(ethane-2,1-diyl)carbamoyl(3-hydroxy-6-methyl-2-oxopyridine-4,1(2H)-diyl)]})tetraacetic acid (7.9 mg, 7.32 μmol) and tri-tert-butyl (3S,10S,14R)-1-[5-(aminomethyl)pyridin-2-yl]-1,4,12-trioxo-3-[(quinolin-2-yl)methyl]-2,5,11,13-tetraazahexadecane-10,14,16-tricarboxylate (3.00 mg, 3.66 μmol) are dissolved in NMP (610 μL). DIPEA (8.9 μl, 51 μmol) is added. PyAOP (2.86 mg, 5.49 μmol) in NMP 170 μL is added. Reaction mix is diluted with 20% ACN/water/0.1% TFA (8 mL) and the products purified by preparative HPLC (RP-HPLC using Äkta pure system, Column: Phenomenex Luna 5 μm C18(2) 100 Å, 250×21.2 mm, Mobile phase: Water/0.1% TFA; ACN Gradient: 20-70% B over 40 min Flow: 10 mL/min Detection: UV 280/335 nm, tR product: 27 min) afforded 2.5 mg (36% yield) of the target compound.

LC-MS (Method K, gradient: 10-70% B over 3 min): Rₜ=2.25 min; MS (ESIpos): m/z=1884.9 [M+H]⁺

Intermediate 158 D Dimer

### 2-[4-[2-[2-[[1-[2-[[6-[[(1S)-2-[[(5S)-6-tert-butoxy-5-[[(1R)-4-tert-butoxy-1-tert-butoxycarbonyl-4-oxo-butyl]carbamoylamino]-6-oxo-hexyl]amino]-2-oxo-1-(2-quinolylmethyl)ethyl]carbamoyl]-3-pyridyl]methylamino]-2-oxo-ethyl]-3-hydroxy-6-methyl-2-oxo-pyridine-4-carbonyl]amino]ethyl-[3-[2-[[1-[2-[[6-[[(1S)-2-[[(5S)-6-tert-butoxy-5-[[(1R)-4-tert-butoxy-1-tert-butoxycarbonyl-4-oxo-butyl]carbamoylamino]-6-oxo-hexyl]amino]-2-oxo-1-(2-quinolylmethyl)ethyl]carbamoyl]-3-pyridyl]methylamino]-2-oxo-ethyl]-3-hydroxy-6-methyl-2-oxo-pyridine-4-carbonyl]amino]ethyl-[2-[[1-(carboxymethyl)-3-hydroxy-6-methyl-2-oxo-pyridine-4-carbonyl]amino]ethyl]amino]propyl]amino]ethylcarbamoyl]-3-hydroxy-6-methyl-2-oxo-1-pyridyl]acetic acid

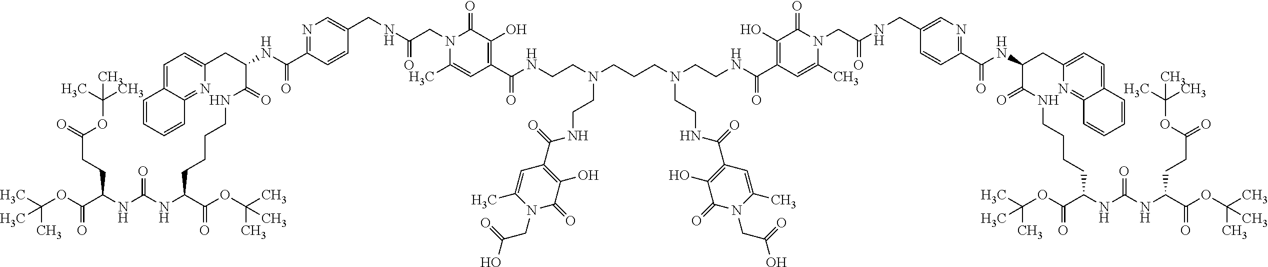 [SMILES: Cc1cc(C(=O)NCCN(CCCN(CCNC(=O)c2cc(C)n(CC(=O)O)c(=O)c2O)CCNC(=O)c2cc(C)n(CC(=O)NCc3ccc(C(=O)N[C@@H](Cc4ccc5ccccc5n4)C(=O)NCCCC[C@H](NC(=O)N[C@H](CCC(=O)OC(C)(C)C)C(=O)OC(C)(C)C)C(=O)OC(C)(C)C)nc3)c(=O)c2O)CCNC(=O)c2cc(C)n(CC(=O)NCc3ccc(C(=O)N[C@@H](Cc4ccc5ccccc5n4)C(=O)NCCCC[C@H](NC(=O)N[C@H](CCC(=O)OC(C)(C)C)C(=O)OC(C)(C)C)C(=O)OC(C)(C)C)nc3)c(=O)c2O)c(O)c(=O)n1CC(=O)O]

2,2′,2″,2′″-(propane-1,3-diylbis{nitrilobis[(ethane-2,1-diyl)carbamoyl(3-hydroxy-6-methyl-2-oxopyridine-4,1(2H)-diyl)]})tetraacetic acid (7.9 mg, 7.32 μmol) and tri-tert-butyl (3S,10S,14R)-1-[5-(aminomethyl)pyridin-2-yl]-1,4,12-trioxo-3-[(quinolin-2-yl)methyl]-2,5,11,13-tetraazahexadecane-10,14,16-tricarboxylate (3.00 mg, 3.66 μmol) are dissolved in NMP (610 μL). DIPEA (8.9 μl, 51 μmol) is added. PyAOP (2.86 mg, 5.49 μmol) in NMP 170 μL is added. Reaction mix is diluted with 20% ACN/water/0.1% TFA (8 mL) and the products purified by preparative HPLC (RP-HPLC using Äkta pure system, Column: Phenomenex Luna 5 μm C18(2) 100 Å, 250×21.2 mm, Mobile phase: Water/0.1% TFA; ACN Gradient: 20-70% B over 40 min Flow: 10 mL/min Detection: UV 280/335 nm, tR product: 28 min) afforded 1.6 mg (16% yield) of the target compound.

LC-MS (Method K, gradient: 10-70% B over 3 min): Rₜ=2.69 min; MS (ESIpos): m/z=1343.7 [M+2H]²⁺

Intermediate 159 D Trimer

### 2-[4-[2-[3-[bis[2-[[1-[2-[[6-[[(1S)-2-[[(5S)-6-tert-butoxy-5-[[(1R)-4-tert-butoxy-1-tert- butoxycarbonyl-4-oxo-butyl]carbamoylamino]-6-oxo-hexyl]amino]-2-oxo-1-(2-quinolylmethyl)ethyl]carbamoyl]-3-pyridyl]methylamino]-2-oxo-ethyl]-3-hydroxy-6-methyl-2-oxo-pyridine-4-carbonyl]amino]ethyl]amino]propyl-[2-[[1-[2-[[6-[[(1S)-2-[[(5S)-6-tert-butoxy-5-[[(1R)-4-tert-butoxy-1-tert-butoxycarbonyl-4-oxo-butyl]carbamoylamino]-6-oxo-hexyl]amino]-2-oxo-1-(2-quinolylmethyl)ethyl]carbamoyl]-3-pyridyl]methylamino]-2-oxo-ethyl]-3-hydroxy-6-methyl-2-oxo-pyridine-4-carbonyl]amino]ethyl]amino]ethylcarbamoyl]-3-hydroxy-6-methyl-2-oxo-1-pyridyl]acetic acid

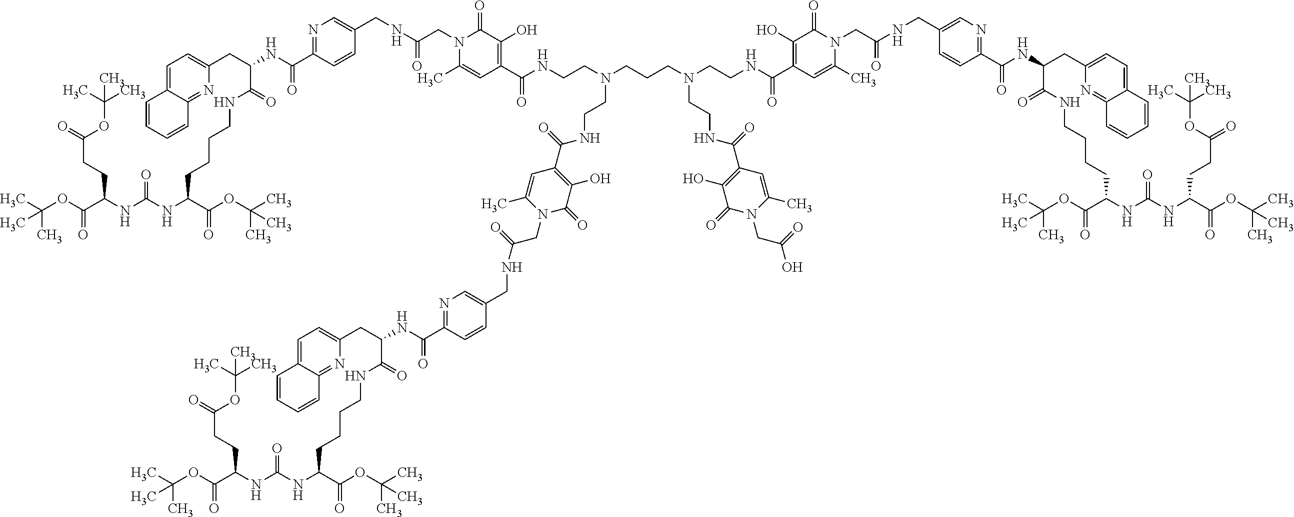 [SMILES: Cc1cc(C(=O)NCCN(CCCN(CCNC(=O)c2cc(C)n(CC(=O)NCc3ccc(C(=O)N[C@@H](Cc4ccc5ccccc5n4)C(=O)NCCCC[C@H](NC(=O)N[C@H](CCC(=O)OC(C)(C)C)C(=O)OC(C)(C)C)C(=O)OC(C)(C)C)nc3)c(=O)c2O)CCNC(=O)c2cc(C)n(CC(=O)NCc3ccc(C(=O)N[C@@H](Cc4ccc5ccccc5n4)C(=O)NCCCC[C@H](NC(=O)N[C@H](CCC(=O)OC(C)(C)C)C(=O)OC(C)(C)C)C(=O)OC(C)(C)C)nc3)c(=O)c2O)CCNC(=O)c2cc(C)n(CC(=O)NCc3ccc(C(=O)N[C@@H](Cc4ccc5ccccc5n4)C(=O)NCCCC[C@H](NC(=O)N[C@H](CCC(=O)OC(C)(C)C)C(=O)OC(C)(C)C)C(=O)OC(C)(C)C)nc3)c(=O)c2O)c(O)c(=O)n1CC(=O)O]

2,2′,2″2′″-(propane-1,3-diylbis{nitrilobis[(ethane-2,1-diyl)carbamoyl(3-hydroxy-6-methyl-2-oxopyridine-4,1(2H)-diyl)]})tetraacetic acid (7.9 mg, 7.32 μmol) and tri-tert-butyl (3S,10S,14R)-1-[5-(aminomethyl)pyridin-2-yl]-1,4,12-trioxo-3-[(quinolin-2-yl)methyl]-2,5,11,13-tetraazahexadecane-10,14,16-tricarboxylate (3.00 mg, 3.66 μmol) are dissolved in NMP (610 μL). DIPEA (8.9 μl, 51 μmol) is added. PyAOP (2.86 mg, 5.49 μmol) in NMP 170 μL is added. Reaction mix is diluted with 20% ACN/water/0.1% TFA (8 mL) and the products purified by preparative HPLC (RP-HPLC using Äkta pure system, Column: Phenomenex Luna 5 μm C18(2) 100 Å, 250×21.2 mm, Mobile phase: Water/0.1% TFA; ACN Gradient: 20-70% B over 40 min

Flow: 10 mL/min Detection: UV 280/335 nm, tR product: 28 min) afforded 0.9 mg (7% yield) of the target compound.

LC-MS (Method K, gradient: 10-70% B over 3 min): Rₜ=2.98 min; MS (ESIpos): m/z=1744.4 [M+2H]²⁺

### Example 34-C-D Monomer

### N⁶—{N-[5-({2-[4-({2-[{3-[bis(2-{[1-(carboxymethyl)-3-hydroxy-6-methyl-2-oxo-1,2-dihydropyridine-4-carbonyl]amino}ethyl)amino]propyl}(2-{[1-(carboxymethyl)-3-hydroxy-6-methyl-2-oxo-1,2-dihydropyridine-4-carbonyl]amino}ethyl)amino]ethyl}carbamoyl)-3-hydroxy-6-methyl-2-oxopyridin-1(2H)-yl]acetamido}methyl)pyridine-2-carbonyl]-3-(quinolin-2-yl)-L-alanyl}-N²-{[(1R)-1,3-dicarboxypropyl]carbamoyl}-L-lysine

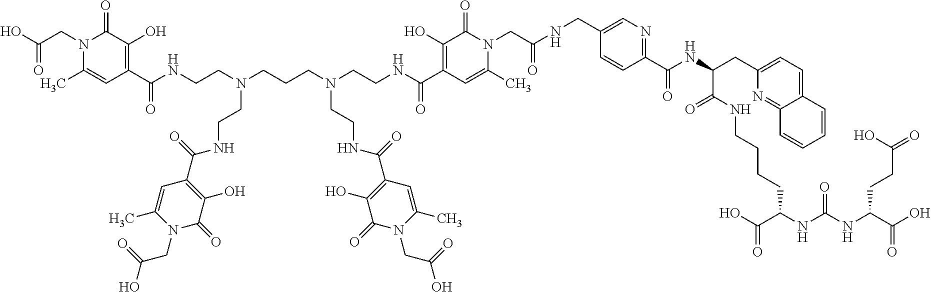 [SMILES: Cc1cc(C(=O)NCCN(CCCN(CCNC(=O)c2cc(C)n(CC(=O)O)c(=O)c2O)CCNC(=O)c2cc(C)n(CC(=O)NCc3ccc(C(=O)N[C@@H](Cc4ccc5ccccc5n4)C(=O)NCCCC[C@H](NC(=O)N[C@H](CCC(=O)O)C(=O)O)C(=O)O)nc3)c(=O)c2O)CCNC(=O)c2cc(C)n(CC(=O)O)c(=O)c2O)c(O)c(=O)n1CC(=O)O]

2,2′-{({3-[(2-{[1-(2-{[(6-{[(7R,11S,18S)-7,11-bis(tert-butoxycarbonyl)-2,2-dimethyl-4,9,17-trioxo-19-(quinolin-2-yl)-3-oxa-8,10,16-triazanonadecan-18-yl]carbamoyl}pyridin-3-yl)methyl]amino}-2-oxoethyl)-3-hydroxy-6-methyl-2-oxo-1,2-dihydropyridine-4-carbonyl]amino}ethyl)(2-{[1-(carboxymethyl)-3-hydroxy-6-methyl-2-oxopyridine-4(2H)-carbonyl]amino}ethyl)amino]propyl}azanediyl)bis[(ethane-2,1-diyl)carbamoyl(3-hydroxy-6-methyl-2-oxopyridine-4,1(2H)-diyl)]}diacetic acid (2.50 mg, 1.33 μmol) is treated with 90% TFA in water (1 mL). Water (15 mL) is added and solution lyophilised.affording 2.30 mg (95% purity, 96% yield) of the target compound.

LC-MS (Method K, gradient: 10-50% B over 3 min): Rₜ=1.78 min; MS (ESIpos): m/z=1716.6 [M+H]⁺

### Example 34-C-D Dimer

### (3S,10S,14R,3'S,10'S,14′R)-1,1′-(propane-1,3-diylbis{[(2-{[1-(carboxymethyl)-3-hydroxy-6-methyl-2-oxo-1,2-dihydropyridine-4-carbonyl]amino}ethyl)azanediyl]ethane-2,1-diylcarbamoyl(3-hydroxy-6-methyl-2-oxopyridine-4,1(2H)-diyl)(1-oxoethane-2,1-diyl)azanediylmethylenepyridine-5,2-diyl})bis{1,4,12-trioxo-3-[(quinolin-2-yl)methyl]-2,5,11,13-tetraazahexadecane-10,14,16-tricarboxylic acid}

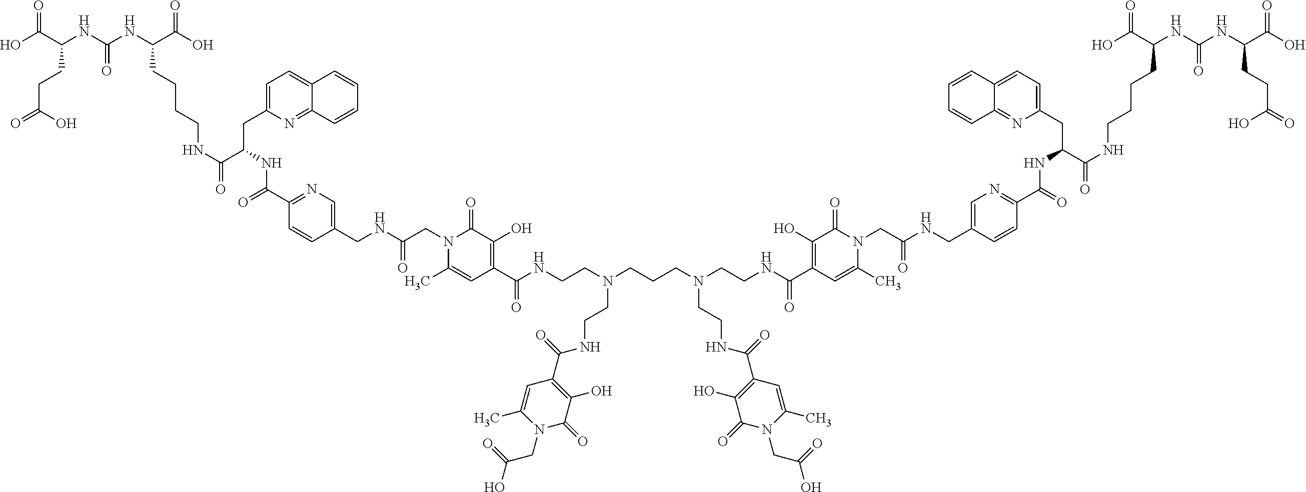 [SMILES: Cc1cc(C(=O)NCCN(CCCN(CCNC(=O)c2cc(C)n(CC(=O)O)c(=O)c2O)CCNC(=O)c2cc(C)n(CC(=O)NCc3ccc(C(=O)N[C@@H](Cc4ccc5ccccc5n4)C(=O)NCCCC[C@H](NC(=O)N[C@H](CCC(=O)O)C(=O)O)C(=O)O)nc3)c(=O)c2O)CCNC(=O)c2cc(C)n(CC(=O)NCc3ccc(C(=O)N[C@@H](Cc4ccc5ccccc5n4)C(=O)NCCCC[C@H](NC(=O)N[C@H](CCC(=O)O)C(=O)O)C(=O)O)nc3)c(=O)c2O)c(O)c(=O)n1CC(=O)O]

2-[4-[2-[2-[[1-[2-[[6-[[(1S)-2-[[(5S)-6-tert-butoxy-5-[[(1R)-4-tert-butoxy-1-tert-butoxycarbonyl-4-oxo-butyl]carbamoylamino]-6-oxo-hexyl]amino]-2-oxo-1-(2-quinolylmethyl)ethyl]carbamoyl]-3-pyridyl]methylamino]-2-oxo-ethyl]-3-hydroxy-6-methyl-2-oxo-pyridine-4-carbonyl]amino]ethyl-[3-[2-[[1-[2-[[6-[[(1S)-2-[[(5S)-6-tert-butoxy-5-[[(1R)-4-tert-butoxy-1-tert-butoxycarbonyl-4-oxo-butyl]carbamoylamino]-6-oxo-hexyl]amino]-2-oxo-1-(2-quinolylmethyl)ethyl]carbamoyl]-3-pyridyl]methylamino]-2-oxo-ethyl]-3-hydroxy-6-methyl-2-oxo-pyridine-4-carbonyl]amino]ethyl-[2-[[1-(carboxymethyl)-3-hydroxy-6-methyl-2-oxo-pyridine-4-carbonyl]amino]ethyl]amino]propyl]amino]ethylcarbamoyl]-3-hydroxy-6-methyl-2-oxo-1-pyridyl]acetic acid (1.60 mg, 0.595 μmol) is treated with 90% TFA in water (1 mL). Water (15 mL) is added and solution lyophilised.affording 1.40 mg (95% purity, 95% yield) of the target compound.

LC-MS (Method K, gradient: 10-50% B over 3 min): Rₜ=2.06 min; MS (ESIpos): m/z=1175.5 [M+2H]²⁺

### Example 34-C-D Trimer

### (3S,10S,14R,3'S,10'S,14′R)-1,1′-{({3-[(2-{[1-(2-{[(6-{[(2S)-1-{[(5S)-5-carboxy-5-({[(1R)-1,3-dicarboxypropyl]carbamoyl}amino)pentyl]amino}-1-oxo-3-(quinolin-2-yl)propan-2-yl]carbamoyl}pyridin-3-yl)methyl]amino}-2-oxoethyl)-3-hydroxy-6-methyl-2-oxopyridine-4(2H)-carbonyl]amino}ethyl)(2-{[1-(carboxymethyl)-3-hydroxy-6-methyl-2-oxo-1,2-dihydropyridine-4-carbonyl]amino}ethyl)amino]propyl}azanediyl)bis[(ethane-2,1-diyl)carbamoyl(3-hydroxy-6-methyl-2-oxopyridine-4,1(2H)-diyl)(1-oxoethane-2,1-diyl)azanediylmethylenepyridine-5,2-diyl]}bis{1,4,12-trioxo-3-[(quinolin-2-yl)methyl]-2,5,11,13-tetraazahexadecane-10,14,16-tricarboxylic acid}

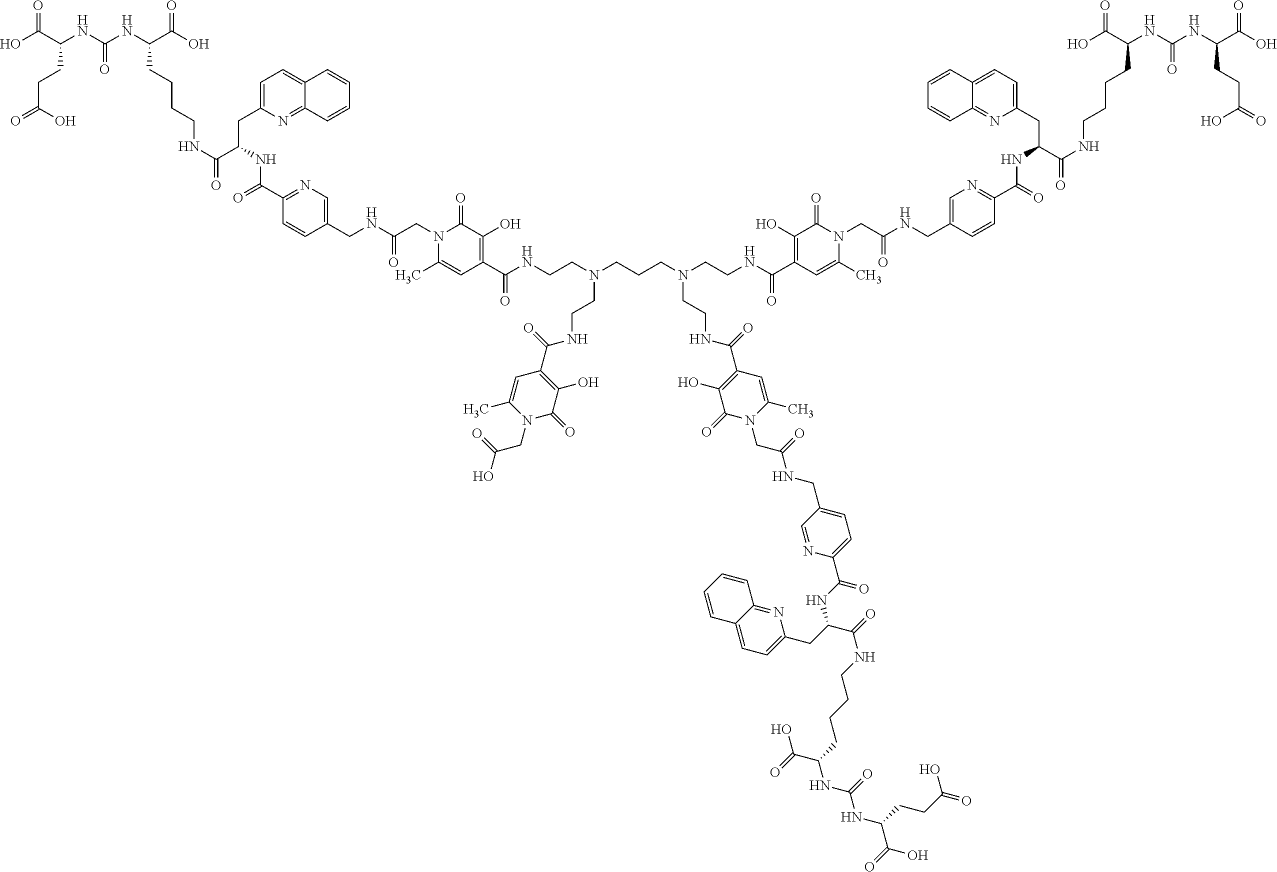 [SMILES: Cc1cc(C(=O)NCCN(CCCN(CCNC(=O)c2cc(C)n(CC(=O)NCc3ccc(C(=O)N[C@@H](Cc4ccc5ccccc5n4)C(=O)NCCCC[C@H](NC(=O)N[C@H](CCC(=O)O)C(=O)O)C(=O)O)nc3)c(=O)c2O)CCNC(=O)c2cc(C)n(CC(=O)NCc3ccc(C(=O)N[C@@H](Cc4ccc5ccccc5n4)C(=O)NCCCC[C@H](NC(=O)N[C@H](CCC(=O)O)C(=O)O)C(=O)O)nc3)c(=O)c2O)CCNC(=O)c2cc(C)n(CC(=O)NCc3ccc(C(=O)N[C@@H](Cc4ccc5ccccc5n4)C(=O)NCCCC[C@H](NC(=O)N[C@H](CCC(=O)O)C(=O)O)C(=O)O)nc3)c(=O)c2O)c(O)c(=O)n1CC(=O)O]

2-[4-[2-[3-[bis[2-[[1-[2-[[6-[[(1S)-2-[[(5S)-6-tert-butoxy-5-[[(1R)-4-tert-butoxy-1-tert- butoxycarbonyl-4-oxo-butyl]carbamoylamino]-6-oxo-hexyl]amino]-2-oxo-1-(2-quinolylmethyl)ethyl]carbamoyl]-3-pyridyl]methylamino]-2-oxo-ethyl]-3-hydroxy-6-methyl-2-oxo-pyridine-4-carbonyl]amino]ethyl]amino]propyl-[2-[[1-[2-[[6-[[(1S)-2-[[(5S)-6-tert-butoxy-5-[[(1R)-4-tert-butoxy-1-tert-butoxycarbonyl-4-oxo-butyl]carbamoylamino]-6-oxo-hexyl]amino]-2-oxo-1-(2-quinolylmethyl)ethyl]carbamoyl]-3-pyridyl]methylamino]-2-oxo-ethyl]-3-hydroxy-6-methyl-2-oxo-pyridine-4-carbonyl]amino]ethyl]amino]ethylcarbamoyl]-3-hydroxy-6-methyl-2-oxo-1-pyridyl]acetic acid (900 μg, 0.26 μmol) is treated with 90% TFA in water (1 mL). Water (15 mL) is added and solution lyophilised.affording 0.8 mg (95% purity, 99% yield) of the target compound.

LC-MS (Method K, gradient: 10-50% B over 3 min): Rₜ=2.14 min; MS (ESIpos): m/z=1491.8 [M+2H]²⁺

### Example 34-C-D Monomer 227Th

### [N⁶—{N-[5-({2-[4-({2-[{3-[bis(2-{[1-(carboxymethyl)-3-(hydroxy-kappaO)-6-methyl-2-(oxo-kappaO)-1,2-dihydropyridine-4-carbonyl]amino}ethyl)amino]propyl}(2-{[1-(carboxymethyl)-3-(hydroxy-kappaO)-6-methyl-2-(oxo-kappaO)-1,2-dihydropyridine-4-carbonyl]amino}ethyl)amino]ethyl}carbamoyl)-3-(hydroxy-kappaO)-6-methyl-2-(oxo-kappaO)pyridin-1(2H)-yl]acetamido}methyl)pyridine-2-carbonyl]-3-(quinolin-2-yl)-L-alanyl}-N²-{[(1R)-1,3-dicarboxypropyl]carbamoyl}-L-lysinato(4−)](²²⁷Th)Thorium

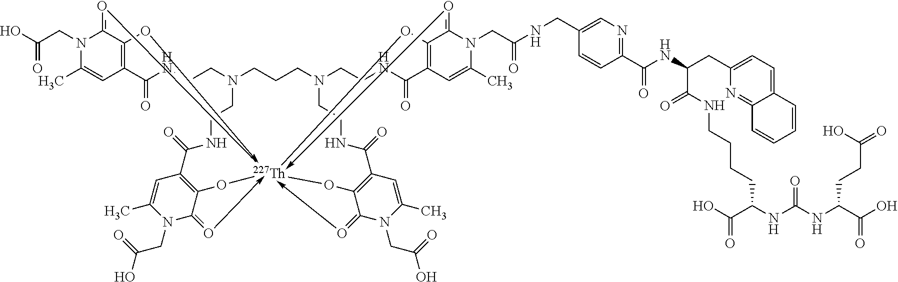

N⁶—{N-[5-({2-[4-({2-[{3-[bis(2-{[1-(carboxymethyl)-3-hydroxy-6-methyl-2-oxo-1,2-dihydropyridine-4-carbonyl]amino}ethyl)amino]propyl}(2-{[1-(carboxymethyl)-3-hydroxy-6-methyl-2-oxo-1,2-dihydropyridine-4-carbonyl]amino}ethyl)amino]ethyl}carbamoyl)-3-hydroxy-6-methyl-2-oxopyridin-1(2H)-yl]acetamido}methyl)pyridine-2-carbonyl]-3-(quinolin-2-yl)-L-alanyl}-N²-{[(1R)-1,3-dicarboxypropyl]carbamoyl}-L-lysine (5.0 μg) dissolved in 158 μL 30 mM citrate buffer (pH 5.5) containing 0.5 mg/mL pABA was mixed with thorium-227 in 0.5 M HCl (2 μL) at 0.3 MBq/nmol specific activity and RAC of 4.6 MBq/mL. 1M carbonate buffer pH 9 (16 μL) was added and mixture incubated for 90 min. The labelling efficiency was determined to be 94% by iTLC.

### Example 34-C-D Dimer 227Th

### (3S,10S,14R,3'S,10'S,14′R)-1,1′-(propane-1,3-diylbis{[(2-{[1-(carboxymethyl)-3-(hydroxy-kappaO)-6-methyl-2-(oxo-kappaO)-1,2-dihydropyridine-4-carbonyl]amino}ethyl)azanediyl]ethane-2,1-diylcarbamoyl[3-(hydroxy-kappaO)-6-methyl-2-(oxo-kappaO)pyridine-4,1(2H)-diyl](1-oxoethane-2,1-diyl)azanediylmethylenepyridine-5,2-diyl})bis{1,4,12-trioxo-3-[(quinolin-2-yl)methyl]-2,5,11,13-tetraazahexadecane-10,14,16-tricarboxylato}(4−)(²²⁷Th)Thorium

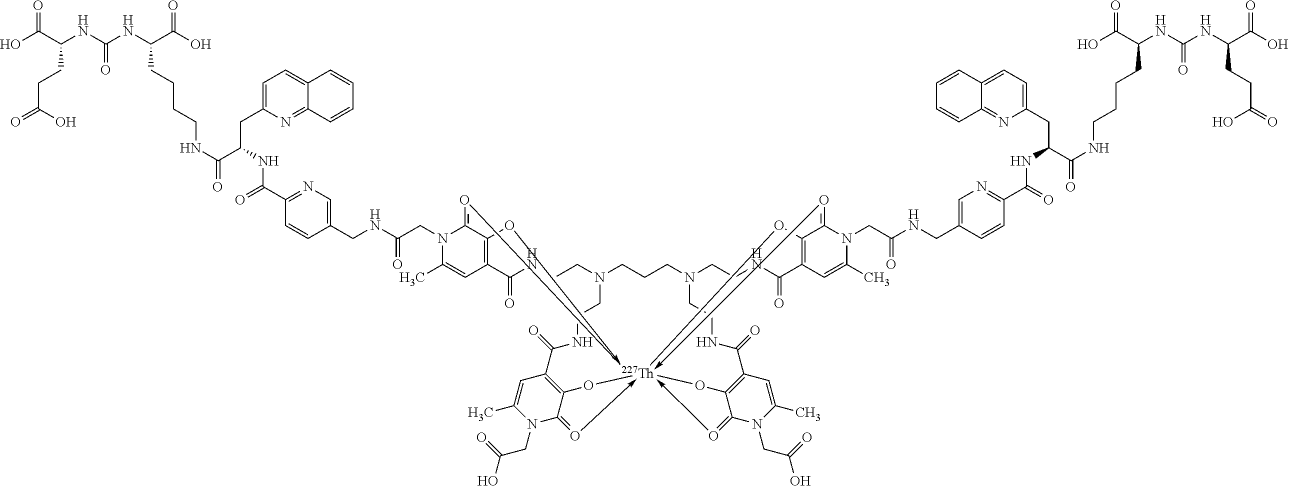

(3S,10S,14R,3'S,10'S,14′R)-1,1′-(propane-1,3-diylbis{[(2-{[1-(carboxymethyl)-3-hydroxy-6-methyl-2-oxo-1,2-dihydropyridine-4-carbonyl]amino}ethyl)azanediyl]ethane-2,1-diylcarbamoyl(3-hydroxy-6-methyl-2-oxopyridine-4,1(2H)-diyl)(1-oxoethane-2,1-diyl)azanediylmethylenepyridine-5,2-diyl})bis{1,4,12-trioxo-3-[(quinolin-2-yl)methyl]-2,5,11,13-tetraazahexadecane-10,14,16-tricarboxylic acid} (6.0 μg) dissolved in 166 μL 30 mM citrate buffer (pH 5.5) containing 0.5 mg/mL pABA was mixed with thorium-227 in 0.5 M HCl (2 μL) at 0.3 MBq/nmol specific activity and RAC of 4.6 MBq/mL. 1M carbonate buffer pH 9 (17 μL) was added and mixture incubated for 90 min. The labelling efficiency was determined to be 95% by iTLC.

### Example 34-C-D Trimer 227Th

### (3S,10S,14R,3'S,10'S,14′R)-1,1′-{({3-[(2-{[1-(2-{[(6-{[(2S)-1-{[(5S)-5-carboxy-5-({[(1R)-1,3-dicarboxypropyl]carbamoyl}amino)pentyl]amino}-1-oxo-3-(quinolin-2-yl)propan-2-yl]carbamoyl}pyridin-3-yl)methyl]amino}-2-oxoethyl)-3-(hydroxy-kappaO)-6-methyl-2-(oxo-kappaO)pyridine-4(2H)-carbonyl]amino}ethyl)(2-{[1-(carboxymethyl)-3-(hydroxy-kappaO)-6-methyl-2-(oxo-kappaO)-1,2-dihydropyridine-4-carbonyl]amino}ethyl)amino]propyl}azanediyl)bis[(ethane-2,1-diyl)carbamoyl[3-(hydroxy-kappaO)-6-methyl-2-(oxo-kappaO)pyridine-4,1(2H)-diyl](1-oxoethane-2,1-diyl)azanediylmethylenepyridine-5,2-diyl]}bis{1,4,12-trioxo-3-[(quinolin-2-yl)methyl]-2,5,11,13-tetraazahexadecane-10,14,16-tricarboxylato}(4−)(²²⁷Th)Thorium

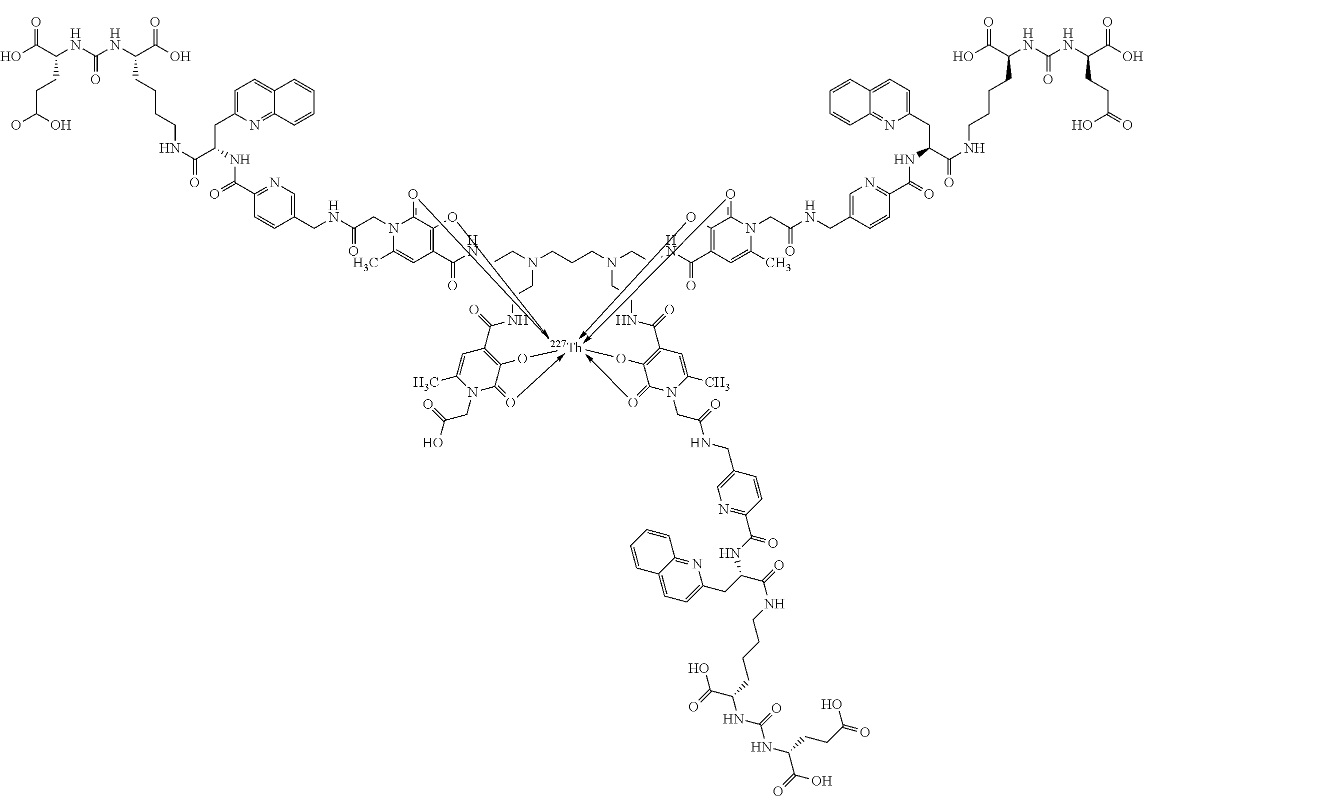

(3S,10S,14R,3'S,10'S,14′R)-1,1′-{({3-[(2-{[1-(2-{[(6-{[(2S)-1-{[(5S)-5-carboxy-5-({[(1R)-1,3-dicarboxypropyl]carbamoyl}amino)pentyl]amino}-1-oxo-3-(quinolin-2-yl)propan-2-yl]carbamoyl}pyridin-3-yl)methyl]amino}-2-oxoethyl)-3-hydroxy-6-methyl-2-oxopyridine-4(2H)-carbonyl]amino}ethyl)(2-{[1-(carboxymethyl)-3-hydroxy-6-methyl-2-oxo-1,2-dihydropyridine-4-carbonyl]amino}ethyl)amino]propyl}azanediyl)bis[(ethane-2,1-diyl)carbamoyl(3-hydroxy-6-methyl-2-oxopyridine-4,1(2H)-diyl)(1-oxoethane-2,1-diyl)azanediylmethylenepyridine-5,2-diyl]}bis{1,4,12-trioxo-3-[(quinolin-2-yl)methyl]-2,5,11,13-tetraazahexadecane-10,14,16-tricarboxylic acid} (8.0 μg) dissolved in 162 μL 30 mM citrate buffer (pH 5.5) containing 0.5 mg/mL pABA was mixed with thorium-227 in 0.5 M HCl (2 μL) at 0.3 MBq/nmol specific activity and RAC of 4.6 MBq/mL. 1M carbonate buffer pH 9 (16 μL) was added and mixture incubated for 90 min. The labelling efficiency was determined to be 68% by iTLC.

### Example 34-C Monomer ⁸⁹Zr

### [N6-{N-[5-({2-[4-({2-[{3-[bis(2-{[1-(carboxymethyl)-3-(hydroxy-kappaO)-6-methyl-2-oxo-1,2-dihydropyridine-4-carbonyl]amino}ethyl)amino]propyl}(2-{[1-(carboxymethyl)-3-(hydroxy-kappaO)-6-methyl-2-oxo-1,2-dihydropyridine-4-carbonyl]amino}ethyl)amino]ethyl}carbamoyl)-3-(hydroxy-kappaO)-6-methyl-2-oxopyridin-1(2H)-yl]acetamido}methyl)pyridine-2-carbonyl]-3-(quinolin-2-yl)-L-alanyl}-N2-{[(1S)-1,3-dicarboxypropyl]carbamoyl}-L-lysinato(4−)](89Zr)Zirconium

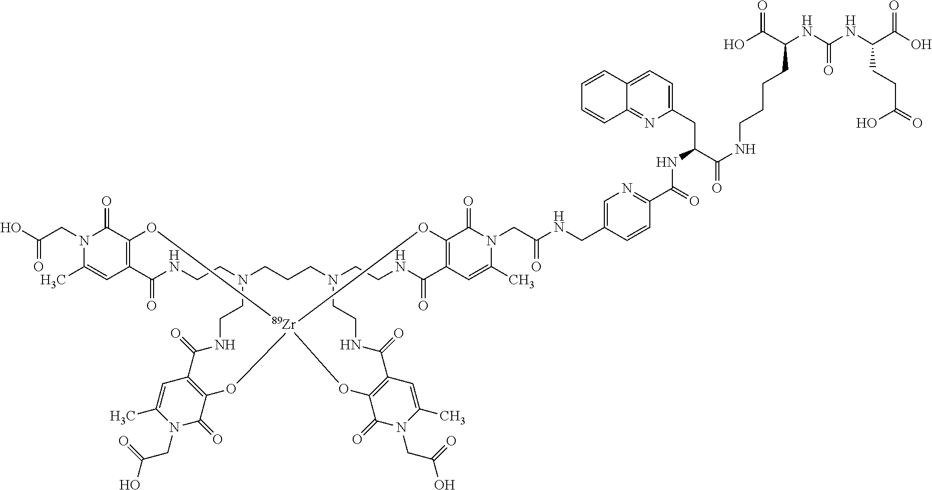

25 μl Zr(Ox)₂ (˜43 MBq) were transferred into a 2 ml Eppendorf tube, then 200 μl 1M HEPES pH8/0.02% TWEEN 20 were added and vortexed gently on circular tube shaker (@˜550 rpm) for 3 minutes @ RT. Then 50 μl N6-{N-[5-({2-[4-({2-[{3-[bis(2-{[1-(carboxymethyl)-3-hydroxy-6-methyl-2-oxo-1,2-dihydropyridine-4-carbonyl]amino}ethyl)amino]propyl}(2-{[1-(carboxymethyl)-3-hydroxy-6-methyl-2-oxo-1,2-dihydropyridine-4-carbonyl]amino}ethyl)amino]ethyl}carbamoyl)-3-hydroxy-6-methyl-2-oxopyridin-1(2H)-yl]acetamido}methyl)pyridine-2-carbonyl]-3-(quinolin-2-yl)-L-alanyl}-N2-{[(1S)-1,3-dicarboxypropyl]carbamoyl}-L-lysine dissolved in 30 mM Citrate pH 5.5, 70 mM NaCl, 0.5 mg/ml pABA, 2 mM EDTA was added (2.9 μg; 1.69 nmol). Then 725 μl 0.1M HEPES pH7.5 was added to give 1000 μl total volume. Incubation was performed by vortex gently on circular tube shaker (@˜550 rpm) for 90 minutes @ room temperature. Purification was performed on C18 cartridge and fractions analyzed by HPLC. The product containing fraction (90 μL) was diluted with 810 μl saline and 130 μL of the resulting solution again checked via HPLC. to determine 15.56 MBq of the final product in the remaining 770 μl saline solution.

### Example 34-C Dimer⁸⁹Zr

### (3S,10S,14S,3'S,10'S,14'S)-1,1′-(propane-1,3-diylbis{[(2-{[1-(carboxymethyl)-3-(hydroxy-kappaO)-6-methyl-2-oxo-1,2-dihydropyridine-4-carbonyl]amino}ethyl)azanediyl]ethane-2,1-diylcarbamoyl[3-(hydroxy-kappaO)-6-methyl-2-oxopyridine-4,1(2H)-diyl](1-oxoethane-2,1-diyl)azanediylmethylenepyridine-5,2-diyl})bis{1,4,12-trioxo-3-[(quinolin-2-yl)methyl]-2,5,11,13-tetraazahexadecane-10,14,16-tricarboxylato}(4−)(⁸⁹Zr)Zirconium

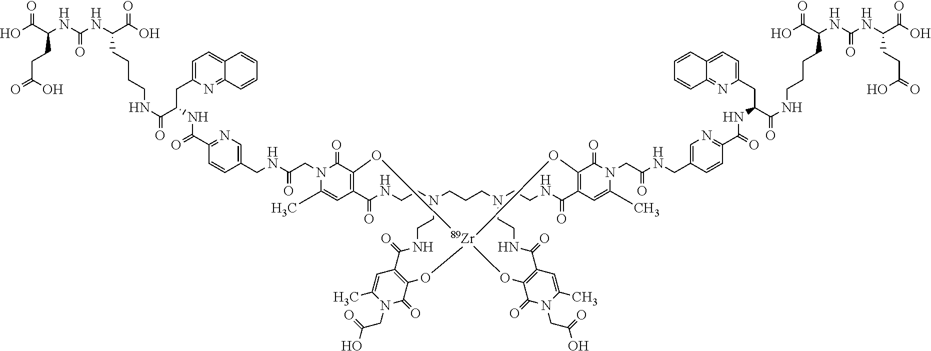

30 μl Zr(Ox)₂ (˜51 MBq) were transferred into a 2 ml Eppendorf tube, then 240 μl 1M HEPES pH8/0.02% TWEEN 20 were added and vortexed gently on circular tube shaker (@˜550 rpm) for 3 minutes @ RT. Then 68 μl (3S,10S,14S,3'S,10'S,14'S)-1,1′-(propane-1,3-diylbis{[(2-{[1-(carboxymethyl)-3-hydroxy-6-methyl-2-oxo-1,2-dihydropyridine-4-carbonyl]amino}ethyl)azanediyl]ethane-2,1-diylcarbamoyl(3-hydroxy-6-methyl-2-oxopyridine-4,1(2H)-diyl)(1-oxoethane-2,1-diyl)azanediylmethylenepyridine-5,2-diyl})bis{1,4,12-trioxo-3-[(quinolin-2-yl)methyl]-2,5,11,13-tetraazahexadecane-10,14,16-tricarboxylic acid} dissolved in 30 mM Citrate pH 5.5, 70 mM NaCl, 0.5 mg/ml pABA, 2 mM EDTA was added (3.97 μg; 1.69 nmol). Then 662 μl 0.1M HEPES pH7.5 was added to give 1000 μl total volume. Incubation was performed by vortex gently on circular tube shaker (@˜550 rpm) for 90 minutes @ room temperature. Purification was performed on C18 cartridge and fractions analyzed by HPLC. The product containing fraction (115 μL) was diluted with 1035 μl saline and 165 μL of the resulting solution again checked via HPLC to determine 21.18 MBq of the final product in the remaining 985 μl saline solution.

### Example 34-C Trimer ⁸⁹Zr

### (3S,10S,14S,3'S,10'S,14'S)-1,1′-{({3-[(2-{[1-(2-{[(6-{[(2S)-1-{[(5S)-5-carboxy-5-({[(1S)-1,3-dicarboxypropyl]carbamoyl}amino)pentyl]amino}-1-oxo-3-(quinolin-2-yl)propan-2-yl]carbamoyl}pyridin-3-yl)methyl]amino}-2-oxoethyl)-3-(hydroxy-kappaO)-6-methyl-2-oxopyridine-4(2H)-carbonyl]amino}ethyl)(2-{[1-(carboxymethyl)-3-(hydroxy-kappaO)-6-methyl-2-oxo-1,2-dihydropyridine-4-carbonyl]amino}ethyl)amino]propyl}azanediyl)bis[(ethane-2,1-diyl)carbamoyl[3-(hydroxy-kappaO)-6-methyl-2-oxopyridine-4,1(2H)-diyl](1-oxoethane-2,1-diyl)azanediylmethylenepyridine-5,2-diyl]}bis{1,4,12-trioxo-3-[(quinolin-2-yl)methyl]-2,5,11,13-tetraazahexadecane-10,14,16-tricarboxylato}(4−)(⁸⁹Zr)Zirconium

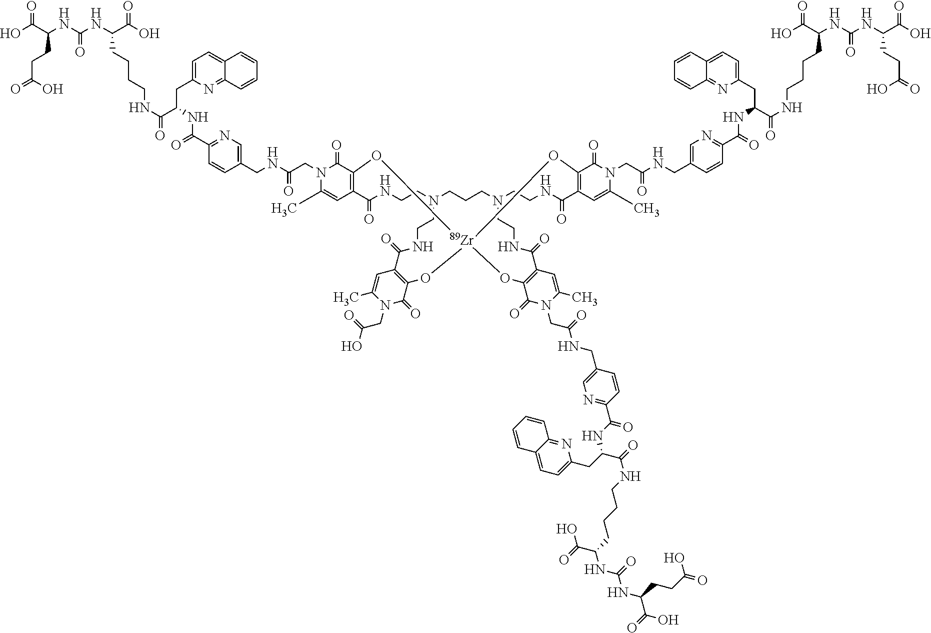

30 μl Zr(Ox)₂ (˜46 MBq) were transferred into a 2 ml Eppendorf tube, then 240 μl 1M HEPES pH8/0.02% TWEEN 20 were added and vortexed gently on circular tube shaker (@˜550 rpm) for 3 minutes @ RT. Then 87 μl (3S,10S,14S,3'S,10'S,14'S)-1,1′-{({3-[(2-{[1-(2-{[(6-{[(2S)-1-{[(5S)-5-carboxy-5-({[(1S)-1,3-dicarboxypropyl]carbamoyl}amino)pentyl]amino}-1-oxo-3-(quinolin-2-yl)propan-2-yl]carbamoyl}pyridin-3-yl)methyl]amino}-2-oxoethyl)-3-hydroxy-6-methyl-2-oxopyridine-4(2H)-carbonyl]amino}ethyl)(2-{[1-(carboxymethyl)-3-hydroxy-6-methyl-2-oxo-1,2-dihydropyridine-4-carbonyl]amino}ethyl)amino]propyl}azanediyl)bis[(ethane-2,1-diyl)carbamoyl(3-hydroxy-6-methyl-2-oxopyridine-4,1(2H)-diyl)(1-oxoethane-2,1-diyl)azanediylmethylenepyridine-5,2-diyl]}bis{1,4,12-trioxo-3-[(quinolin-2-yl)methyl]-2,5,11,13-tetraazahexadecane-10,14,16-tricarboxylic acid} dissolved in 30 mM Citrate pH 5.5, 70 mM NaCl, 0.5 mg/ml pABA, 2 mM EDTA was added (5.05 μg; 1.69 nmol). Then 643 μl 0.1M HEPES pH7.5 was added to give 1000 μl total volume. Incubation was performed by vortexing gently on circular tube shaker (@˜550 rpm) for 90 minutes @ room temperature. Purification was performed on C18 cartridge and fractions analyzed by HPLC. The product containing fraction (120 μL) was diluted with 1080 μl saline and 120 μL of the resulting solution again checked with HPLC to determine 21 MBq of the final product in the remaining 985 μl saline solution.

#35 Linker

Intermediate 160

### methyl 4-methoxynaphthalene-2-carboxylate

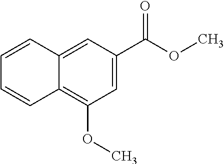 [SMILES: COC(=O)c1cc(OC)c2ccccc2c1]

To a solution of 4-hydroxynaphthalene-2-carboxylic acid (5.00 g, 26.6 mmol) in N,N-dimethylformamide (100 ml) were added potassium carbonate (11.0 g, 79.7 mmol) and iodomethane (8.3 ml, 130 mmol) at room temperature. After stirring at room temperature for 12 hours. Water was added to the reaction mixture. The mixture was extracted with ethyl acetate. The organic layer was evaporated under reduced pressure to give a residue. The residue was purified by chromatography (1000 mesh, petroleum ether:ethyl acetate=1:0, then 100:1) to give methyl 4-methoxy-2-naphthoate (5.70 g, 97% purity, 96% yield) as a yellow oil.

LC-MS (Method D): Rₜ=0.841 min; MS (ESIpos): m/z=217.0 [M+H]⁺.

¹H NMR (400 MHz, DMSO-d₆) δ=8.22 (s, 1H), 8.20-8.16 (m, 1H), 8.11-8.05 (m, 1H), 7.68-7.59 (m, 2H), 7.34 (d, 1H), 4.02 (s, 3H), 3.91 (s, 3H).

Intermediate 161

### (4-methoxynaphthalen-2-yl)MeOH

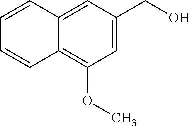

To a solution of methyl 4-methoxynaphthalene-2-carboxylate (5.70 g, 97% purity, 25.6 mmol) in toluene (70 ml) was added diisobutylaluminum hydride (38 ml, 1M in toluene, 38 mmol) at −40° C. After stirring at 0° C. for 1 hours, the mixture was quenched with saturated ammonium chloride and extracted with ethyl acetate. The organic phase was washed with brine, dried over anhydrous sodium sulfate, filtered and concentrated to give a residue. The residue was purified by column chromatography (1000 mesh, petroleum ether:ethyl acetate=20:1, then 10:1) to give (4-methoxy-2-naphthyl)methanol (5.00 g, 97% purity) as a yellow solid.

LC-MS (Method D): Rₜ=0.728 min; MS (ESIpos): m/z=189.0 [M+H]⁺.

¹H NMR (400 MHz, DMSO-d₆) δ=8.12 (d, 1H), 7.83 (d, 1H), 7.54-7.37 (m, 3H), 6.95 (s, 1H), 5.33 (t, 1H), 4.67 (d, 2H), 3.97 (s, 3H).

Intermediate 162

### 4-methoxynaphthalene-2-carbaldehyde

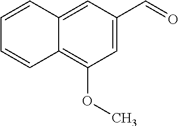

To a solution of (4-methoxynaphthalen-2-yl)methanol (5.00 g, 97% purity, 25.8 mmol) in dichloromethane (50 ml) was added 3,3,3-triacetoxy-3-iodophthalide (13.1 g, 30.9 mmol) at room temperature. After stirring at 25° C. for 1 hour, the mixture was filtered through a pad of celite and the filtrate was concentrated to give a residue. The residue was purified by column chromatography (1000 mesh, petroleum ether:ethyl acetate=100:1 then 50:1) to give 4-methoxy-2-naphthaldehyde (4.30 g, 98% purity, 88% yield) as a light yellow solid.

LC-MS (Method D): Rₜ=0.797 min; MS (ESIpos): m/z=187.0 [M+H]⁺.

¹H NMR (400 MHz, DMSO-d₆) δ=10.09 (s, 1H), 8.23-8.15 (m, 2H), 8.14-8.07 (m, 1H), 7.75-7.64 (m, 2H), 7.24 (s, 1H), 4.03 (s, 3H).

Intermediate 163

### methyl (2Z)-2-{[(benzyloxy)carbonyl]amino}-3-(4-methoxynaphthalen-2-yl)prop-2-enoate

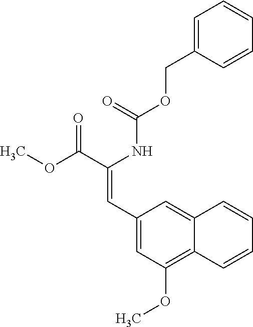 [SMILES: COC(=O)/C(=C/c1cc(OC)c2ccccc2c1)NC(=O)OCc1ccccc1]

1,8-Diazabicyclo(5.4.0)undec-7-ene (0.470 ml, 3.2 mmol) was added dropwise to a solution of methyl {[(benzyloxy)carbonyl]amino}(dimethoxyphosphoryl)acetate (1.05 g, 3.16 mmol) in dichloromethane (10 ml). After stirring at room temperature for 10 minutes, a solution of 4-methoxynaphthalene-2-carbaldehyde (500 mg, 98% purity, 2.63 mmol) in dichloromethane (5 ml) was added. The reaction mixture was stirred at room temperature for 2 hours. The reaction mixture was concentrated and diluted with ethyl acetate (40 ml). The solution was washed with hydrochloric acid (1M) and brine. The organic phase was dried over anhydrous sodium sulfate and filtered. The filtrate was concentrated and purified by column chromatography (1000 mesh, petroleum ether:ethyl acetate=10:1, then 5:1, then 3:1) to give a residue. The residue was triturated with methyl tertiary butyl ether (10 ml). The suspension was filtered and the filter cake was collected to give methyl (2Z)-2-{[(benzyloxy)carbonyl]amino}-3-(4-methoxy-2-naphthyl)acrylate (700 mg, 95% purity, 65% yield) as a white solid.

LC-MS (Method D): Rₜ=0.899 min; MS (ESIpos): m/z=392.1 [M+H]⁺.

¹H NMR (400 MHz, DMSO-d₆) δ=9.33 (br.s, 1H), 8.17-8.04 (m, 1H), 7.91-7.68 (m, 2H), 7.64-7.49 (m, 2H), 7.48-6.94 (m, 7H), 5.13 (s, 2H), 3.88 (s, 3H), 3.75 (s, 3H).

Intermediate 164

### methyl (2R)-2-{[(benzyloxy)carbonyl]amino}-3-(4-methoxynaphthalen-2-yl)propanoate

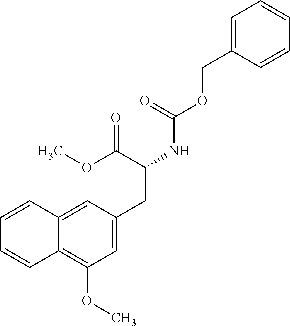 [SMILES: COC(=O)[C@@H](Cc1cc(OC)c2ccccc2c1)NC(=O)OCc1ccccc1]

To a solution of methyl (2Z)-2-{[(benzyloxy)carbonyl]amino}-3-(4-methoxynaphthalen-2-yl)prop-2-enoate (3.80 g, 98% purity, 9.51 mmol) in methanol (200 ml) were added (R)—[Rh(COD)(MaxPhos)]BF₄ (0.06 eq) (338 mg, 571 μmol). The reaction mixture was stirred at room temperature for 16 hours under hydrogen atmosphere (2.5 MPa). The reaction mixture was evaporated under reduced pressure to give a residue. The residue was dissolved in dichloromethane, the solution was filtered through silica gel (200-300 mesh). The filtrate was evaporated under reduced pressure to give methyl (2S)-2-{[(benzyloxy)carbonyl]amino}-3-(4-methoxy-2-naphthyl)propanoate (4.00 g, 94% purity, 100% yield) as a yellow oil.

LC-MS (Method D): Rₜ=0.874 min; MS (ESIpos): m/z=394.1 [M+H]⁺.

Intermediate 165

### (2R)-2-{[(benzyloxy)carbonyl]amino}-3-(4-methoxynaphthalen-2-yl)propanoic acid

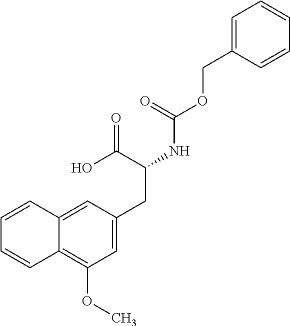 [SMILES: COc1cc(C[C@@H](NC(=O)OCc2ccccc2)C(=O)O)cc2ccccc12]

To a solution of methyl (2R)-2-{[(benzyloxy)carbonyl]amino}-3-(4-methoxynaphthalen-2-yl)propanoate (4.00 g, 94% purity, 9.56 mmol) in pyridine (70 ml, 870 mmol) was added lithium iodide anhydrous (3.84 g, 28.7 mmol) at room temperature. The reaction mixture was heated to 110° C. and stirred at 110° C. for 12 hours. The reaction mixture was cooled to room temperature, concentrated and dissolved in methanol. The solution was adjusted to pH 5-6 through hydrochloric acid (1M). The solution was evaporated under reduced pressure and purified by preparative HPLC (Instrument: Shimadzu LC-20AP; Column: Phenomenex luna C18 250*100 mm*10 μm; eluent A: 0.225% formic acid in water, eluent B: acetonitrile; gradient: 0-48 min 40-70% B; flow 400 ml/min; temperature: room temperature; Detector: UV 220/254 nm) to give (2S)-2-{[(benzyloxy)carbonyl]amino}-3-(4-methoxy-2-naphthyl)propanoic acid (2.60 g, 99% purity, 71% yield) as a yellow solid.

LC-MS (Method D): Rₜ=0.853 min; MS (ESIpos): m/z=380.0 [M+H]⁺.

¹H NMR (400 MHz, DMSO-d₆) δ=12.76 (s, 1H), 8.09 (d, 1H), 7.74 (dd, 2H), 7.55-7.42 (m, 2H), 7.32 (s, 1H), 7.29-7.09 (m, 5H), 6.93 (s, 1H), 4.96 (s, 2H), 4.41-4.30 (m, 1H), 3.93 (s, 3H), 3.23 (dd, 1H), 2.99 (dd, 1H).

Intermediate 166

### tri-tert-butyl (5R,12S,16S)-5-[(4-methoxynaphthalen-2-yl)methyl]-3,6,14-trioxo-1-phenyl-2-oxa-4,7,13,15-tetraazaoctadecane-12,16,18-tricarboxylate

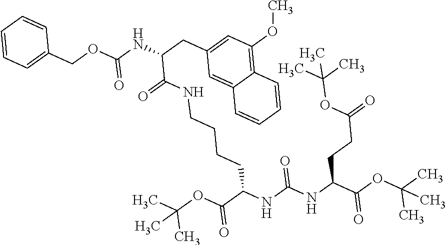 [SMILES: COc1cc(C[C@@H](NC(=O)OCc2ccccc2)C(=O)NCCCC[C@H](NC(=O)N[C@@H](CCC(=O)OC(C)(C)C)C(=O)OC(C)(C)C)C(=O)OC(C)(C)C)cc2ccccc12]

di-tert-butyl N-{[(2R)-6-amino-1-tert-butoxy-1-oxohexan-2-yl]carbamoyl}-L-glutamate (626 mg, 98% purity, 1.26 mmol) and (2S)-2-{[(benzyloxy)carbonyl]amino}-3-(4-methoxynaphthalen-2-yl)propanoic acid (477 mg, 1.26 mmol) were solubilised in DMF (9.7 ml), 4-methylmorpholine (550 μl, 5.0 mmol, CAS-RN: 109-02-4) and HATU (718 mg, 1.89 mmol) were added and the mixture was stirred under argon for 2 h at rt. The mixture was diluted with brine and extracted with DCM. The organic phase was dried, evaporated and purified by preparative HPLC (C18, acetonitrile/water with 0.1% formic acid) to give 541 mg (95% purity, 48% yield) of the target compound.

LC-MS (Method 1): Rₜ=1.59 min; MS (ESIpos): m/z=850 [M+H]⁺

¹H-NMR (400 MHz, DMSO-d6) δ[ppm]: 1.382 (12.58), 1.387 (16.00), 2.518 (1.13), 2.522 (0.74), 3.930 (1.97), 4.905 (0.44), 4.929 (0.44), 6.929 (0.44), 7.223 (0.49), 7.227 (0.43), 7.235 (0.64), 7.240 (0.48), 7.297 (0.40).

Intermediate 167

### di-tert-butyl (2S)-2-({[(2S)-6-{[(2R)-2-amino-3-(4-methoxynaphthalen-2-yl)propanoyl]amino}-1-tert-butoxy-1-oxohexan-2-yl]carbamoyl}amino)pentanedioate

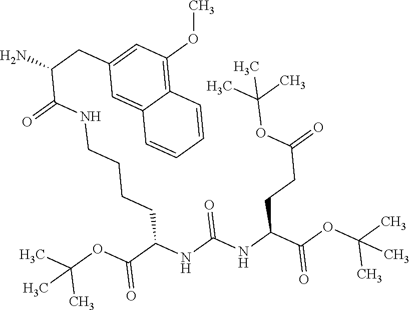 [SMILES: COc1cc(C[C@@H](N)C(=O)NCCCC[C@H](NC(=O)N[C@@H](CCC(=O)OC(C)(C)C)C(=O)OC(C)(C)C)C(=O)OC(C)(C)C)cc2ccccc12]

tri-tert-butyl (5R,12S,16S)-5-[(4-methoxynaphthalen-2-yl)methyl]-3,6,14-trioxo-1-phenyl-2-oxa-4,7,13,15-tetraazaoctadecane-12,16,18-tricarboxylate (540 mg, 90% purity, 572 μmol) was solubilised in MeOH (4.6 ml), Palladium on carbon (6.09 mg, 10% purity, 57.2 μmol) was added and the mixture was purged with hydrogen. The mixture was stirred for 3 h at rt under hydrogen atmosphere. The mixture was filtered over Celite, washed with MeOH and evaporated to give 380 mg (95% purity, 88% yield) of the target compound.

LC-MS (Method 1): Rₜ=1.21 min; MS (ESIpos): m/z=716 [M+H]⁺

¹H-NMR (400 MHz, DMSO-d6) δ[ppm]: 1.381 (16.00), 1.385 (13.49), 1.388 (11.58), 2.518 (0.79), 2.522 (0.53), 2.727 (0.67), 2.729 (0.67), 2.888 (0.81), 3.022 (0.42), 3.036 (0.42), 3.951 (3.67), 6.831 (0.64), 6.834 (0.65), 7.249 (0.62).

Intermediate 168

### tri-tert-butyl (3R,10S,14S)-1-[(1r,4S)-4-({[(benzyloxy)carbonyl]amino}methyl)cyclohexyl]-3-[(4-methoxynaphthalen-2-yl)methyl]-1,4,12-trioxo-2,5,11,13-tetraazahexadecane-10,14,16-tricarboxylate

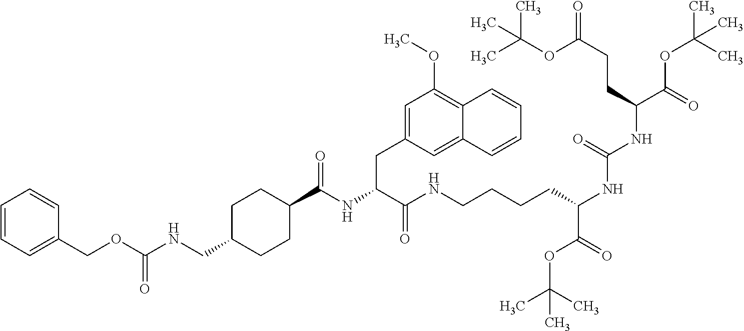 [SMILES: COc1cc(C[C@@H](NC(=O)[C@H]2CC[C@H](CNC(=O)OCc3ccccc3)CC2)C(=O)NCCCC[C@H](NC(=O)N[C@@H](CCC(=O)OC(C)(C)C)C(=O)OC(C)(C)C)C(=O)OC(C)(C)C)cc2ccccc12]

di-tert-butyl (2S)-2-({[(2S)-6-{[(2R)-2-amino-3-(4-methoxynaphthalen-2-yl)propanoyl]amino}-1-tert-butoxy-1-oxohexan-2-yl]carbamoyl}amino)pentanedioate (379 mg, 95% purity, 504 μmol) and (1r,4r)-4-({[(benzyloxy)carbonyl]amino}methyl)cyclohexane-1-carboxylic acid (147 mg, 504 μmol) were solubilised in DMF (3.9 ml), 4-methylmorpholine (220 μl, 2.0 mmol, CAS-RN: 109-02-4) and HATU (287 mg, 755 μmol) were added and the mixture was stirred under argon 1 h at rt. The mixture was diluted with brine and extracted with DCM. The organic phase was dried, evaporated and purified by preparative HPLC (C18, acetonitrile/water with 0.1% formic acid) to give 338 mg (90% purity, 61% yield) of the target compound.

LC-MS (Method 1): Rₜ=1.57 min; MS (ESIpos): m/z=989 [M+H]⁺

¹H-NMR (400 MHz, DMSO-d6) δ[ppm]: 1.376 (15.76), 1.384 (16.00), 2.518 (1.26), 2.522 (0.87), 2.539 (14.58), 2.801 (0.39), 3.420 (0.71), 3.426 (0.55), 3.433 (0.47), 3.941 (3.15), 4.985 (1.50), 6.867 (0.63), 7.237 (0.79), 7.315 (0.47), 7.318 (0.47), 7.329 (1.10), 7.336 (1.18), 7.351 (0.47), 7.355 (0.47).

Intermediate 169

### tri-tert-butyl (3R,10S,14S)-3-[(4-methoxy-2-naphthyl)methyl]-1-{trans-4-[2-(methylamino)ethyl]cyclohexyl}-1,4,12-trioxo-2,5,11,13-tetraazahexadecane-10,14,16-tricarboxylate

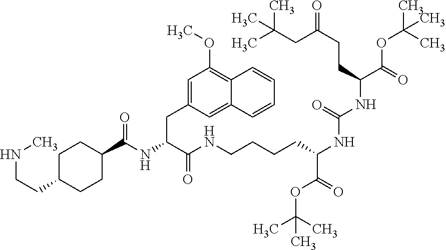 [SMILES: CNCC[C@H]1CC[C@H](C(=O)N[C@H](Cc2cc(OC)c3ccccc3c2)C(=O)NCCCC[C@H](NC(=O)N[C@@H](CCC(=O)CC(C)(C)C)C(=O)OC(C)(C)C)C(=O)OC(C)(C)C)CC1]

tri-tert-butyl (3R,10S,14S)-1-[(1r,4S)-4-({[(benzyloxy)carbonyl]amino}methyl)cyclohexyl]-3-[(4-methoxynaphthalen-2-yl)methyl]-1,4,12-trioxo-2,5,11,13-tetraazahexadecane-10,14,16-tricarboxylate (335 mg, 90% purity, 305 μmol) was solubilised in MeOH (2.5 ml), Palladium on carbon (33.0 mg, 10% purity, 31 μmol) was added and the mixture was purged with hydrogen.

The mixture was stirred at rt under hydrogen atmosphere. The mixture was filtered over Celite, washed with MeOH and evaporated. The residue was purified by preparative HPLC (C18, acetonitrile/water with 0.1% formic acid) to give 29.0 mg (11% yield) of the target compound.

LC-MS (Method 1): Rt=1.22 min; MS (ESIpos): m/z=883 [M+H]+

¹H-NMR (400 MHz, DMSO-d6) δ[ppm]: 1.380 (16.00), 1.389 (15.25), 1.905 (0.61), 1.940 (0.62), 1.958 (0.59), 2.059 (5.71), 2.331 (0.41), 2.518 (2.26), 2.523 (1.41), 2.673 (0.41), 3.946 (3.37), 6.874 (0.66), 7.241 (0.65), 7.928 (0.52).

### Example 35-A

### (3R,10S,14S)-3-[(4-methoxynaphthalen-2-yl)methyl]-1-{(1r,4S)-4-[2-(methylamino)ethyl]cyclohexyl}-1,4,12-trioxo-2,5,11,13-tetraazahexadecane-10,14,16-tricarboxylic acid

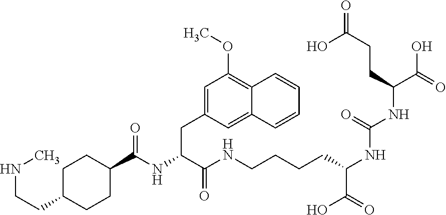 [SMILES: CNCC[C@H]1CC[C@H](C(=O)N[C@H](Cc2cc(OC)c3ccccc3c2)C(=O)NCCCC[C@H](NC(=O)N[C@@H](CCC(=O)O)C(=O)O)C(=O)O)CC1]

tri-tert-butyl (3R,10S,14S)-3-[(4-methoxynaphthalen-2-yl)methyl]-1-{(1r,4S)-4-[2-(methylamino)ethyl]cyclohexyl}-1,4,12-trioxo-2,5,11,13-tetraazahexadecane-10,14,16-tricarboxylate (11.1 mg, 12.6 μmol) was solubilised in DCM (400 μl), TFA (190 μl, 2.5 mmol) was added and the mixture was stirred under argon overnight at rt. The mixture was evaporated and purified by preparative HPLC (C18, acetonitrile/water with 0.1% formic acid) to give 4.50 mg (90% purity, 45% yield) of the target compound.

LC-MS (Method 1): Rt=0.77 min; MS (ESIpos): m/z=715 [M+H]⁺

¹H-NMR (400 MHz, DMSO-d6) δ[ppm]: 0.795 (0.39), 1.232 (1.63), 1.287 (0.46), 1.352 (0.52), 1.433 (0.52), 1.629 (0.46), 1.761 (0.65), 1.907 (1.11), 2.202 (0.59), 2.224 (1.04), 2.245 (0.85), 2.275 (2.42), 2.332 (2.81), 2.336 (1.24), 2.518 (16.00), 2.522 (10.06), 2.673 (2.81), 2.678 (1.24), 2.903 (0.39), 3.022 (0.65), 3.037 (0.52), 3.063 (0.52), 3.076 (0.59), 3.098 (0.46), 3.110 (0.46), 3.946 (6.92), 3.973 (0.46), 3.985 (0.52), 4.002 (0.52), 4.020 (0.46), 6.336 (0.46), 6.356 (0.46), 6.886 (1.31), 7.254 (1.24), 7.404 (0.59), 7.407 (0.72), 7.424 (0.59), 7.428 (0.59), 7.447 (0.52), 7.450 (0.52), 7.468 (0.72), 7.724 (0.78), 7.744 (0.65), 7.876 (0.39), 8.033 (0.46), 8.045 (0.85), 8.066 (0.72).

#36 Linker

Intermediate 170

### isoquinoline-3-carbaldehyde

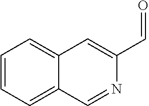 [SMILES: O=Cc1cc2ccccc2cn1]

To a mixture of methyl isoquinoline-3-carboxylate (7.80 g, 41.7 mmol) in toluene (160 ml) was added diisobutylaluminum hydride (1M in toluene, 63 ml, 63 mmol) dropwise at −60° C. under nitrogen atmosphere. The reaction mixture was stirred at −60° C. for 2 hours. The mixture was quenched with saturated ammonium chloride solution and saturated potassium tartrate. After stirring at room temperature for 16 hours, the mixture was extracted with ethyl acetate. The combined organic layers were washed with brine, dried over anhydrous sodium sulfate, filtered and concentrated under reduced pressure to give a residue. The residue was purified by column chromatography (200-300 mesh, petroleum ether:ethyl acetate=50:1 to 1:1) to give isoquinoline-3-carbaldehyde (2.60 g, 90% purity, 36% yield) as a yellow solid.

¹H-NMR (400 MHz, CDCl₃): δ [ppm]=10.30 (s, 1H), 9.42 (s, 1H), 9.43 (s, 1H), 8.11 (dd, 1H), 8.06 (dd, 1H), 7.86-7.82 (m, 2H).

Intermediate 171

### methyl (2Z)-2-{[(benzyloxy)carbonyl]amino}-3-(isoquinolin-3-yl)prop-2-enoate

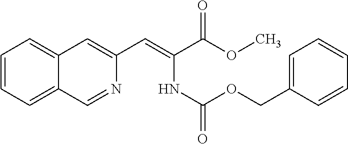 [SMILES: COC(=O)/C(=C/c1cc2ccccc2cn1)NC(=O)OCc1ccccc1]

To a solution of methyl {[(benzyloxy)carbonyl]amino}(dimethoxyphosphoryl)acetate (10.9 g, 33.0 mmol) in dichloromethane (100 ml) was added 1,8-diazabicyclo(5.4.0)undec-7-ene (4.9 ml, 33 mmol) dropwise at 0-5° C. After stirring for 30 minutes, a solution of isoquinoline-3-carbaldehyde (4.80 g, 90% purity, 27.5 mmol) in dichloromethane (100 ml) was added dropwise at 0-5° C. After stirring at room temperature for 2 hours, the reaction mixture (combined with another batch) was diluted with water and extracted with ethyl acetate. The organic phase was washed with brine, dried over anhydrous sodium sulfate, filtered and concentrated in vacuo. The residue was purified by column chromatography on silica gel (200-300 mesh, petroleum ether:ethyl acetate=20:1 to 5:1) to give methyl (2Z)-2-{[(benzyloxy)carbonyl]amino}-3-(isoquinolin-3-yl)acrylate (7.20 g, 99% purity, 72% yield) as a yellow solid.

¹H-NMR (400 MHz, CDCl₃): δ [ppm]=11.05 (s, 1H), 9.13 (s, 1H), 7.89 (d, 1H), 7.73 (d, 1H), 7.64 (td, 1H), 7.56-7.52 (m, 2H), 7.34-7.28 (m, 5H), 6.45 (s, 1H), 5.13 (s, 2H), 3.78 (s, 3H).

Intermediate 172

### (rac)-methyl N-[(benzyloxy)carbonyl]-3-isoquinolin-3-yl-alaninate

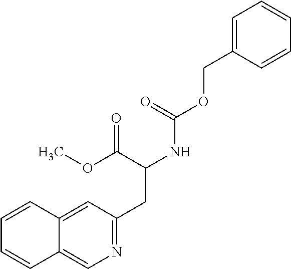 [SMILES: COC(=O)C(Cc1cc2ccccc2cn1)NC(=O)OCc1ccccc1]

To a solution of (2Z)-2-{[(benzyloxy)carbonyl]amino}-3-(isoquinolin-3-yl)prop-2-enoate (4.70 g, 99% purity, 12.8 mmol) and nickel(II) chloride hexahydrate (305 mg, 1.28 mmol) in methanol (120 ml)/tetrahydrofuran (40 ml) was added sodium borohydride (1.46 g, 38.5 mmol) in three portions at 0° C. After stirring at room temperature for 16 hours, the reaction mixture (combined with another batch) was diluted with water and extracted with ethyl acetate. The organic phase was washed with brine, dried over sodium sulfate, filtered and the filtrate was concentrated under reduce pressure to afford methyl N-[(benzyloxy)carbonyl]-3-isoquinolin-3-ylalaninate 4.45 g (90% purity, 86% yield) as a yellow oil.

LC-MS (Method C): Rₜ=0.782 min; MS (ESIpos): m/z=365.2 [M+H]⁺.

Intermediate 173

### (rac)-N-[(benzyloxy)carbonyl]-3-isoquinolin-3-yl-alanine

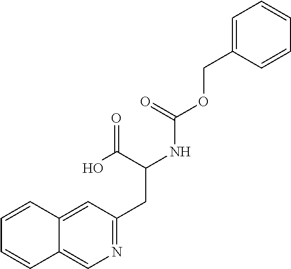 [SMILES: O=C(NC(Cc1cc2ccccc2cn1)C(=O)O)OCc1ccccc1]

To a solution of methyl N-[(benzyloxy)carbonyl]-3-isoquinolin-3-yl-alaninate (3.85 g, 90% purity, 9.51 mmol) in THF (100 ml) and water (100 ml) was added lithium hydroxide (1.20 g, 28.5 mmol). The reaction mixture was stirred at rt for 2 h. The reaction mixture was acidified to pH=3 by HCl (6 M), diluted with water and extracted with EtOAc. The organic phase was washed with brine, dried and concentrated in vacuo to give 2.20 g (94% purity, 62% yield) of the target compound.

Intermediate 174

### N-[(benzyloxy)carbonyl]-3-isoquinolin-3-yl-L-alanine

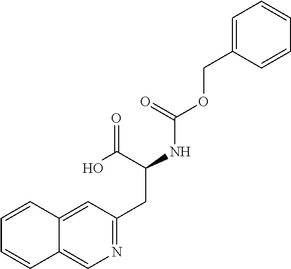 [SMILES: O=C(N[C@@H](Cc1cc2ccccc2cn1)C(=O)O)OCc1ccccc1]

(Rac)N-[(benzyloxy)carbonyl]-3-isoquinolin-3-yl-L-alanine (2.20 g, 94% purity, 5.90 mmol) was separated by preparative-SFC (Instrument: Waters 80Q SFC; Column: Chiralcel OD column, 250×25 mm×10 μm particle size; Mobile Phase: Phase A for Supercritical carbon dioxide, Phase B for ethanol (0.1% ammonia water); Isocratic elution: 40% Phase B (60% Phase A); flow: 70 g/min; cycle time: 4.6 minutes; Back Pressure: 100 bar to keep the carbon dioxide in Supercritical flow; temperature: room temperature; Detector: UV 220 nm) to give (Ent)N-[(benzyloxy)carbonyl]-3-isoquinolin-3-ylalanine (stereoisomer 1, first eluting, SFC retention time: 1.595, 865 mg, 97% purity) as an off-white solid and (Ent)N-[(benzyloxy)carbonyl]-3-isoquinolin-3-ylalanine (stereoisomer 2, second eluting, SFC retention time: 1.969, specific rotation (calc.): −9.458°, concentration: 0.1882 g/100 ml in methanol, 25° C., 698 mg, 97% purity) as a white solid. SFC (Method: Cellucoat-Isopropanol (diethylamine)-20-3 ml-35T) Instrument: SHIMADZU-2020; Column: Cellucoat 50×4.6 mm I.D., 3 μm Mobile phase: 20% iso-propanol (0.05% diethylamine) in carbon dioxide from 5% to 40%; Flow rate: 3 mL/min; temperature: 35° C.; Detector: 220 nm.

LC-MS (Method C): Rₜ=0.637 min; MS (ESIpos): m/z=351.0 [M+H]⁺.

¹H-NMR (400 MHz, DMSO-d₆): δ [ppm]=9.27 (s, 1H), 8.10 (d, 1H), 7.88 (d, 1H), 7.76 (td, 1H), 7.65-7.62 (m, 3H), 7.26-7.21 (m, 5H), 4.98-4.91 (m, 2H), 4.60-4.56 (m, 1H), 3.37-3.36 (m, 1H), 3.19-3.13 (m, 1H).

LC-MS (Method C): Rₜ=0.637 min; MS (ESIpos): m/z=351.1 [M+H]⁺.

¹H-NMR (400 MHz, DMSO-d₆): δ [ppm]=9.27 (s, 1H), 8.10 (d, 1H), 7.88 (d, 1H), 7.76 (td, 1H), 7.67-7.63 (m, 3H), 7.26-7.21 (m, 5H), 4.98-4.91 (m, 2H), 4.60-4.56 (m, 1H), 3.37-3.36 (m, 1H), 3.19-3.13 (m, 1H).

Intermediate 175

### tri-tert-butyl (5S,12S,16S)-5-[(isoquinolin-3-yl)methyl]-3,6,14-trioxo-1-phenyl-2-oxa-4,7,13,15-tetraazaoctadecane-12,16,18-tricarboxylate

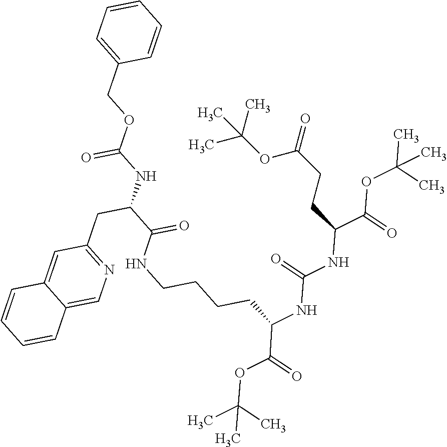 [SMILES: CC(C)(C)OC(=O)CC[C@H](NC(=O)N[C@@H](CCCCNC(=O)[C@H](Cc1cc2ccccc2cn1)NC(=O)OCc1ccccc1)C(=O)OC(C)(C)C)C(=O)OC(C)(C)C]

di-tert-butyl N-{[(2S)-6-amino-1-tert-butoxy-1-oxohexan-2-yl]carbamoyl}-L-glutamate (615 mg, 1.26 mmol) and N-[(benzyloxy)carbonyl]-3-isoquinolin-3-yl-L-alanine (442 mg, 1.26 mmol) were solubilised in DMF (9.7 ml), 4-methylmorpholine (350 μl, 3.2 mmol, CAS-RN: 109-02-4) and HATU (671 mg, 1.77 mmol) were added and the mixture was stirred under argon overnight at rt. The mixture was diluted with DCM/propan-2-ol and washed with water and brine. The organic layer was dried, evaporated and purified by flash chromatography (SiO2, DCM/Ethanol gradient 0%-5%) to give 400 mg (100% purity, 39% yield) of the target compound.

LC-MS (Method 1): Rt=1.45 min; MS (ESIpos): m/z=821 [M+H]⁺

Intermediate 176

### di-tert-butyl (2S)-2-({[(2S)-6-{[(2S)-2-amino-3-(isoquinolin-3-yl)propanoyl]amino}-1-tert-butoxy-1-oxohexan-2-yl]carbamoyl}amino)pentanedioate

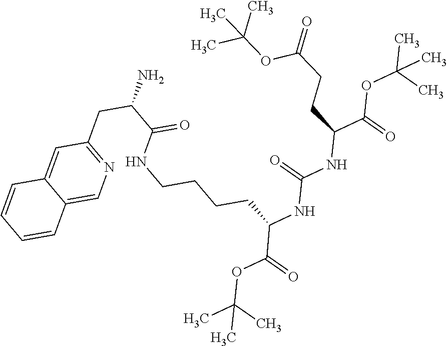 [SMILES: CC(C)(C)OC(=O)CC[C@H](NC(=O)N[C@@H](CCCCNC(=O)[C@@H](N)Cc1cc2ccccc2cn1)C(=O)OC(C)(C)C)C(=O)OC(C)(C)C]

tri-tert-butyl (5S,12S,16S)-5-[(isoquinolin-3-yl)methyl]-3,6,14-trioxo-1-phenyl-2-oxa-4,7,13,15-tetraazaoctadecane-12,16,18-tricarboxylate (400 mg, 488 μmol) was solubilised in MeOH (4.0 ml), Palladium on carbon (51.9 mg, 10% purity, 48.8 μmol) was added and the mixture was purged with hydrogen. The mixture was stirred for 6 h at rt under hydrogen atmosphere. The mixture was filtered over Celite, washed with MeOH and evaporated to give 300 mg (94% purity, 84% yield) of the target compound.

LC-MS (Method 1): Rt=1.12 min; MS (ESIpos): m/z=687 [M+H]⁺

¹H-NMR (400 MHz, DMSO-d6) δ[ppm]: 1.380 (16.00), 1.384 (14.04), 1.387 (12.11), 1.396 (2.81), 2.518 (0.65), 2.522 (0.41), 7.620 (0.67), 7.883 (0.46), 9.249 (0.71).

Intermediate 177

### tri-tert-butyl (3S,10S,14S)-1-[(1r,4S)-4-({[(benzyloxy)carbonyl]amino}methyl)cyclohexyl]-3-[(isoquinolin-3-yl)methyl]-1,4,12-trioxo-2,5,11,13-tetraazahexadecane-10,14,16-tricarboxylate

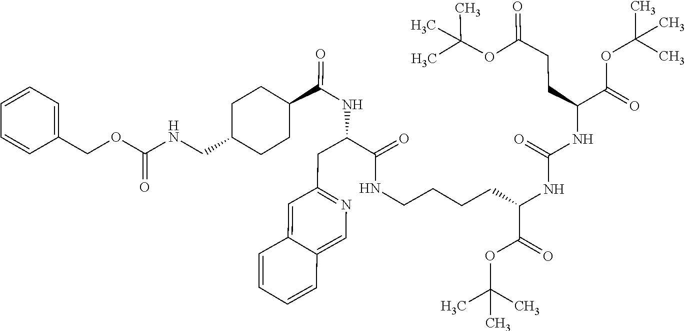 [SMILES: CC(C)(C)OC(=O)CC[C@H](NC(=O)N[C@@H](CCCCNC(=O)[C@H](Cc1cc2ccccc2cn1)NC(=O)[C@H]1CC[C@H](CNC(=O)OCc2ccccc2)CC1)C(=O)OC(C)(C)C)C(=O)OC(C)(C)C]

di-tert-butyl (2S)-2-({[(2S)-6-{[(2S)-2-amino-3-(isoquinolin-3-yl)propanoyl]amino}-1-tert-butoxy-1-oxohexan-2-yl]carbamoyl}amino)pentanedioate (300 mg, 437 μmol) and (1r,4r)-4-({[(benzyloxy)carbonyl]amino}methyl)cyclohexane-1-carboxylic acid (127 mg, 437 μmol) were solubilised in DMF (5 ml), 4-methylmorpholine (140 μl, 1.3 mmol, CAS-RN: 109-02-4) and HATU (200 mg, 525 μmol) were added and the mixture was stirred under argon overnight at rt. The mixture was evaporated and purified by preparative HPLC (C18, acetonitrile/water with 0.1% formic acid) to give 165 mg (95% purity, 37% yield) of the target compound.

LC-MS (Method 1): Rₜ=1.46 min; MS (ESIpos): m/z=960 [M+H]⁺

¹H-NMR (400 MHz, DMSO-d6) δ[ppm]: 1.231 (0.50), 1.379 (15.54), 1.381 (16.00), 2.332 (0.88), 2.518 (4.87), 2.522 (3.17), 2.673 (0.90), 4.984 (1.28), 7.319 (0.42), 7.331 (0.99), 7.337 (1.05), 7.356 (0.40), 7.577 (0.61), 7.823 (0.44), 9.226 (0.71).

Intermediate 178

### tri-tert-butyl (3S,10S,14S)-1-[(1r,4S)-4-(aminomethyl)cyclohexyl]-3-[(isoquinolin-3-yl)methyl]-1,4,12-trioxo-2,5,11,13-tetraazahexadecane-10,14,16-tricarboxylate

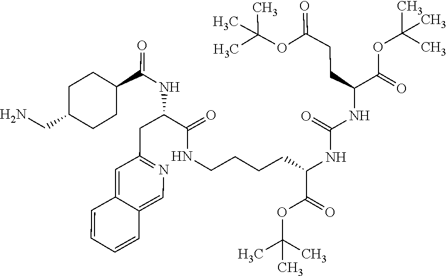 [SMILES: CC(C)(C)OC(=O)CC[C@H](NC(=O)N[C@@H](CCCCNC(=O)[C@H](Cc1cc2ccccc2cn1)NC(=O)[C@H]1CC[C@H](CN)CC1)C(=O)OC(C)(C)C)C(=O)OC(C)(C)C]

tri-tert-butyl (3S,10S,14S)-1-[(1r,4S)-4-({[(benzyloxy)carbonyl]amino}methyl)cyclohexyl]-3-[(isoquinolin-3-yl)methyl]-1,4,12-trioxo-2,5,11,13-tetraazahexadecane-10,14,16-tricarboxylate (170 mg, 177 μmol) was solubilised in MeOH (1.4 ml), Palladium on carbon (18.9 mg, 10% purity, 17.7 μmol) was added and the mixture was purged with hydrogen. The mixture was stirred for 8 h at rt under hydrogen atmosphere. The mixture was filtered over Celite, washed with MeOH and evaporated to give 120 mg (95% purity, 78% yield) of the target compound.

LC-MS (Method 1): Rt=1.09 min; MS (ESIpos): m/z=826 [M+H]⁺

¹H-NMR (400 MHz, DMSO-d6) δ[ppm]: 1.231 (0.59), 1.380 (16.00), 2.203 (0.47), 2.327 (1.25), 2.331 (0.92), 2.518 (5.46), 2.523 (3.39), 2.669 (1.23), 2.673 (0.88), 3.109 (0.51), 7.580 (0.69), 9.226 (0.59).

### Example 36-A

### N⁶—{N-[(1r,4S)-4-(aminomethyl)cyclohexane-1-carbonyl]-3-(isoquinolin-3-yl)-L-alanyl}-N²-{[(1S)-1,3-dicarboxypropyl]carbamoyl}-L-lysine

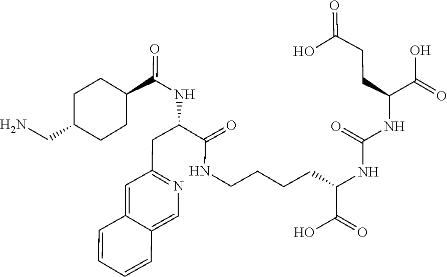 [SMILES: NC[C@H]1CC[C@H](C(=O)N[C@@H](Cc2cc3ccccc3cn2)C(=O)NCCCC[C@H](NC(=O)N[C@@H](CCC(=O)O)C(=O)O)C(=O)O)CC1]

tri-tert-butyl (3S,10S,14S)-1-[(1r,4S)-4-(aminomethyl)cyclohexyl]-3-[(isoquinolin-3-yl)methyl]-1,4,12-trioxo-2,5,11,13-tetraazahexadecane-10,14,16-tricarboxylate (25.0 mg, 30.3 μmol) was solubilised in DCM (970 μl), TFA (580 μl, 7.6 mmol) was added and the mixture was stirred under argon overnight at rt. The mixture was evaporated and purified by preparative HPLC (C18, acetonitrile/water with 0.1% formic acid) to give 4.50 mg (99% purity, 22% yield) of the target compound.

LC-MS (Method 1): Rt=0.54 min; MS (ESIpos): m/z=657 [M+H]⁺

¹H-NMR (400 MHz, DMSO-d6) δ[ppm]: 0.858 (2.88), 1.046 (1.85), 1.073 (1.85), 1.153 (4.74), 1.172 (9.55), 1.189 (6.66), 1.214 (5.70), 1.230 (5.70), 1.275 (4.19), 1.291 (4.12), 1.415 (3.30), 1.448 (3.23), 1.558 (1.99), 1.642 (4.67), 1.684 (3.78), 1.741 (4.26), 1.751 (2.88), 1.779 (2.40), 1.986 (15.66), 2.056 (2.13), 2.073 (14.90), 2.083 (4.19), 2.156 (1.51), 2.171 (2.82), 2.186 (2.88), 2.204 (2.13), 2.239 (2.95), 2.255 (1.99), 2.518 (16.00), 2.522 (9.96), 2.539 (1.72), 2.574 (5.49), 2.589 (5.01), 2.934 (2.88), 2.952 (3.50), 2.967 (3.50), 3.044 (5.08), 3.067 (5.36), 3.079 (5.22), 3.102 (4.67), 3.236 (8.93), 3.249 (9.75), 3.269 (10.23), 3.282 (10.64), 3.387 (13.32), 3.923 (4.46), 3.934 (5.29), 3.999 (2.13), 4.016 (4.19), 4.034 (4.05), 4.052 (1.79), 4.677 (2.20), 4.686 (2.61), 4.700 (2.54), 4.722 (1.10), 6.156 (2.06), 6.390 (2.13), 6.409 (2.33), 6.892 (0.96), 7.589 (3.16), 7.607 (10.03), 7.624 (3.85), 7.704 (3.09), 7.724 (5.29), 7.744 (3.30), 7.806 (3.30), 7.830 (6.66), 7.850 (4.46), 8.059 (5.97), 8.080 (5.42), 8.120 (2.61), 8.141 (2.40), 8.213 (0.76), 8.243 (2.06), 9.226 (10.64).

Intermediate 179

### tri-tert-butyl (3S,10S,14S)-3-[(isoquinolin-3-yl)methyl]-1,4,12-trioxo-1-[(1r,4S)-4-({2-[4,7,10-tris(2-tert-butoxy-2-oxoethyl)-1,4,7,10-tetraazacyclododecan-1-yl]acetamido}methyl)cyclohexyl]-2,5,11,13-tetraazahexadecane-10,14,16-tricarboxylate

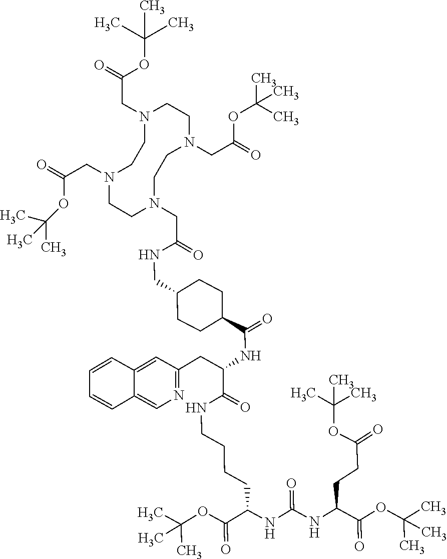 [SMILES: CC(C)(C)OC(=O)CC[C@H](NC(=O)N[C@@H](CCCCNC(=O)[C@H](Cc1cc2ccccc2cn1)NC(=O)[C@H]1CC[C@H](CNC(=O)CN2CCN(CC(=O)OC(C)(C)C)CCN(CC(=O)OC(C)(C)C)CCN(CC(=O)OC(C)(C)C)CC2)CC1)C(=O)OC(C)(C)C)C(=O)OC(C)(C)C]

[4,7,10-tris(2-tert-butoxy-2-oxoethyl)-1,4,7,10-tetraazacyclododecan-1-yl]acetic acid (69.4 mg, 121 μmol; CAS-RN:[137076-54-1]), [(1H-benzotriazol-1-yl)oxy](dimethylamino)-N,N-dimethylmethaniminium hexafluoridophosphate(1−) (45.4 mg, 120 μmol; CAS-RN:94790-37-1) and N,N-diisopropylethylamine (17 μl, 110 μmol) were stirred in DMF (1.2 ml) for 10 min at rt. tri-tert-butyl (3S,10S,14S)-1-[(1r,4S)-4-(aminomethyl)cyclohexyl]-3-[(isoquinolin-3-yl)methyl]-1,4,12-trioxo-2,5,11,13-tetraazahexadecane-10,14,16-tricarboxylate (25.0 mg, 30.3 μmol) was added and the mixture was stirred overnight at rt. The mixture was evaporated and purified by preparative HPLC (C18, acetonitrile/water with 0.1% formic acid) to give 25.0 mg (97% purity, 58% yield) of the target compound.

LC-MS (Method 1): Rt=1.38 min; MS (ESIpos): m/z=1380 [M+H]⁺

### Example 36-B

### N⁶-{3-(isoquinolin-3-yl)-N-[(1r,4S)-4-({2-[4,7,10-tris(carboxymethyl)-1,4,7,10-tetraazacyclododecan-1-yl]acetamido}methyl)cyclohexane-1-carbonyl]-L-alanyl}-N²-{[(1S)-1,3-dicarboxypropyl]carbamoyl}-L-lysine

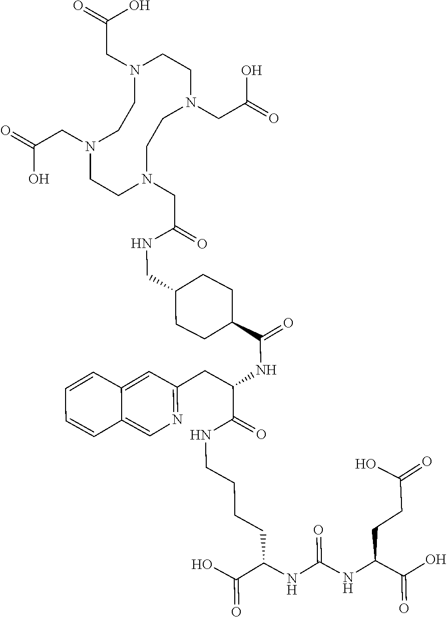 [SMILES: O=C(O)CC[C@H](NC(=O)N[C@@H](CCCCNC(=O)[C@H](Cc1cc2ccccc2cn1)NC(=O)[C@H]1CC[C@H](CNC(=O)CN2CCN(CC(=O)O)CCN(CC(=O)O)CCN(CC(=O)O)CC2)CC1)C(=O)O)C(=O)O]

tri-tert-butyl (3S,10S,14S)-3-[(isoquinolin-3-yl)methyl]-1,4,12-trioxo-1-[(1r,4S)-4-({2-[4,7,10-tris(2-tert-butoxy-2-oxoethyl)-1,4,7,10-tetraazacyclododecan-1-yl]acetamido}methyl)cyclohexyl]-2,5,11,13-tetraazahexadecane-10,14,16-tricarboxylate (25.0 mg, 18.1 μmol) was solubilised in DCM (580 μl), TFA (280 μl, 3.6 mmol) was added and the mixture was stirred under argon over the weekend at rt. The mixture was evaporated and purified by preparative HPLC (C18, acetonitrile/water with 0.1% formic acid) to give 5.00 mg (95% purity, 25% yield) of the target compound.

LC-MS (Method 1): Rt=0.54 min; MS (ESIpos): m/z=1044 [M+H]⁺

¹H-NMR (400 MHz, DMSO-d6) δ[ppm]: 1.232 (1.37), 1.352 (0.44), 2.332 (2.68), 2.336 (1.18), 2.518 (16.00), 2.523 (10.89), 2.539 (1.74), 2.678 (1.25), 2.983 (1.12), 3.072 (0.62), 3.427 (1.37), 7.599 (0.87), 7.726 (0.44), 7.837 (0.56), 8.061 (0.87), 8.081 (0.62), 9.229 (1.12).

### Example 36-B 227Th

### {N⁶-[3-(isoquinolin-3-yl)-N-{(1r,4S)-4-[(2-{4,7,10-tris[(carboxy-kappaO)methyl]-1,4,7,10-tetraazacyclododecan-1-yl-kappa²N¹,N⁴}acetamido)methyl]cyclohexane-1-carbonyl}alanyl]-N²-{[(1S)-1,3-dicarboxypropyl]carbamoyl}-L-lysinato(3−)}(²²⁷Th)Thorium

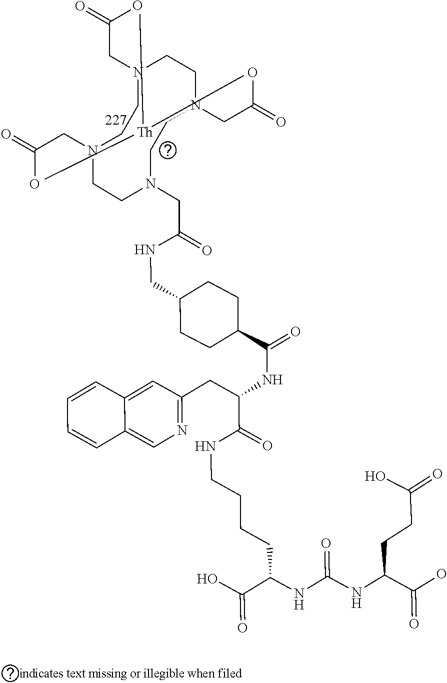

N⁶-{3-(isoquinolin-3-yl)-N-[(1r,4S)-4-({2-[4,7,10-tris(carboxymethyl)-1,4,7,10-tetraazacyclododecan-1-yl]acetamido}methyl)cyclohexane-1-carbonyl]-L-alanyl}-N²-{[(1S)-1,3-dicarboxypropyl]carbamoyl}-L-lysine was dissolved as 10 mM solution in DMSO and diluted to a 250 μM solution in 400 mM sodium acetate buffer (pH 5) containing 0.5 mg/mL pABA. 1.07 μl of this solution was used in a 40 μl labeling reaction. Thorium-227 in 400 mM sodium acetate buffer (pH 5) plus 20% EtOH was added giving a specific activity of 0.375 MBq/nmol and a RAC of 2.5 MBq/mL. The mixture was incubated at 95° C. for 60 min. The labelling efficiency was determined to be 98.5% by iTLC.

#37 Linker

Intermediate 180

### 2-bromo-4-nitronaphthalen-1-amine

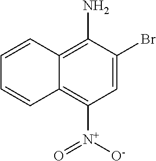 [SMILES: Nc1c(Br)cc([N+](=O)[O-])c2ccccc12]

To a solution of 4-nitronaphthalen-1-amine (6.00 g, 31.9 mmol) in acetonitrile (100 ml) were added ammonium acetate (246 mg, 3.19 mmol) and N-bromosuccinimide (5.96 g, 33.5 mmol) at room temperature. The reaction mixture was stirred at room temperature for 0.5 hour. The reaction mixture was concentrated under reduced pressure to give a residue. The residue was diluted with water and extracted with ethyl acetate. The combined organic layers were washed brine, dried over anhydrous sodium sulfate, filtered and the filtrate was concentrated under reduced pressure to give 2-bromo-4-nitronaphthalen-1-amine (7.00 g 82% yield) as a yellow solid.

LC-MS (Method C): Rₜ=0.895 min; MS (M+H): m/z=267.1

¹H-NMR (400 MHz, DMSO-d₆): δ [ppm]=8.78 (d, 1H), 8.54 (s, 1H), 8.45 (d, 1H), 7.70-7.85 (m, 1H), 7.54-7.64 (m, 1H), 7.50 (s, 2H).

Intermediate 181

### 3-bromo-1-nitronaphthalene

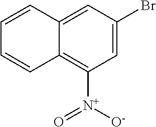 [SMILES: O=[N+]([O-])c1cc(Br)cc2ccccc12]

To a solution of 2-bromo-4-nitronaphthalen-1-amine (5.00 g, 18.7 mmol) in dichloromethane (200 ml) was added boron trifluoride diethyl etherate (3.6 ml) in one portion at −15° C. After stirring for 15 minutes, a solution of tert-butyl nitrite (2.7 ml) in dichloromethane (50 ml) was added dropwise at −15° C. After stirring for 1 hour, hypophosphorous (24.7 g, 50% purity, 187 mmol) acid and copper(I) oxide (133.94 mg) were added. The reaction mixture was stirred at 0° C. for 1 hour. The mixture was neutralized to pH=7 with saturated sodium bicarbonate and extracted with ethyl acetate. The combined organic layers were washed with brine, dried over anhydrous sodium sulfate, filtered and the filtrate was concentrated in vacuum to give a residue. The residue was purified by column chromatography on silica gel (petroleum ether:ethyl acetate=100:1 to 1:1) to give 3-bromo-1-nitronaphthalene (3.50 g, 74% yield) as a brown solid.

¹H NMR (400 MHz, DMSO-d₆): δ [ppm]=8.67 (d, 1H), 8.44 (d, 1H), 8.26 (dd, 1H), 8.23 (dd, 1H), 7.82 (td, 1H), 7.78 (td, 1H).

Intermediate 182

### 3-bromonaphthalen-1-amine

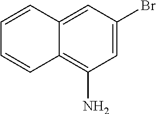 [SMILES: Nc1cc(Br)cc2ccccc12]

To a solution of 3-bromo-1-nitronaphthalene (3.50 g, 13.9 mmol) in a mixed solvent of ethanol (60 ml), tetrahydrofuran (60 ml) and water (60 ml) were added iron (3.10 g, 55.5 mmol) and ammonium chloride (4.46 g, 83.3 mmol) in one portion. After stirring at 75° C. for 2 hours, the reaction mixture was filtered and the filtrate was washed with saturated sodium bicarbonate. The aqueous phase was extracted with ethyl acetate. The combined organic layers were washed with brine, dried over anhydrous sodium sulfate, filtered and the filtrate was concentrated to give a residue. The residue was purified by column chromatography on silica gel (200-300 mesh, petroleum ether:ethyl acetate=1:0 to 10:1) to give 3-bromonaphthalen-1-amine (2.60 g, 84% yield) as a yellow solid.

¹H NMR (400 MHz, DMSO-d₆): δ [ppm]=8.06 (d, 1H), 7.68 (d, 1H), 7.44 (td, 1H), 7.42 (td, 1H), 7.26 (d, 1H), 6.78 (d, 1H), 6.08 (s, 2H).

Intermediate 183

### di-tert-butyl (3-bromonaphthalen-1-yl)-2-imidodicarbonate

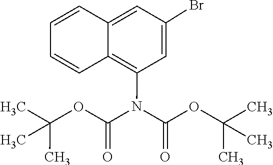 [SMILES: CC(C)(C)OC(=O)N(C(=O)OC(C)(C)C)c1cc(Br)cc2ccccc12]

To a solution of 3-bromonaphthalen-1-amine (2.60 g, 11.7 mmol) in toluene (100 ml) were added 4-dimethylaminopyridine (5.72 g, 46.8 mmol) and di-tert-butyl dicarbonate (8.1 ml, 35 mmol). After stirring at 100° C. for 16 hours, the reaction mixture was poured into water and extracted with ethyl acetate. The combined organic layers were washed with brine, dried over anhydrous sodium sulfate, filtered and the filtrate was concentrated in vacuum to give di-tert-butyl (3-bromo-1-naphthyl)-2-imidodicarbonate (4.00 g, 81% yield) as a yellow solid.

¹H NMR (400 MHz, DMSO-d₆): δ [ppm]=8.26 (d, 1H), 8.03-7.93 (m, 1H), 7.70-7.50 (m, 4H), 1.32 (s, 18H).

Intermediate 184

### tert-butyl (3-bromonaphthalen-1-yl)carbamate

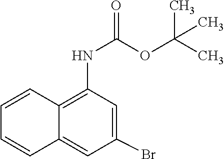 [SMILES: CC(C)(C)OC(=O)Nc1cc(Br)cc2ccccc12]

To a solution of di-tert-butyl (3-bromonaphthalen-1-yl)-2-imidodicarbonate (4.00 g, 9.47 mmol) in methanol (50 ml) was added potassium carbonate (3.27 g, 23.7 mmol) in one portion. After stirring at 25° C. for 16 hours, the reaction mixture was poured into water and extracted with ethyl acetate. The combined organic layers were washed with brine, dried over anhydrous sodium sulfate, filtered and the filtrate was concentrated in vacuum to give tert-butyl (3-bromo-1-naphthyl)carbamate (3.00 g, 98% yield) as a yellow solid.

¹H NMR (400 MHz, DMSO-d₆): δ [ppm]=9.51 (s, 1H), 8.14-8.06 (m, 1H), 7.99-7.78 (m, 3H), 7.61-7.48 (m, 2H), 1.50 (s, 9H).

Intermediate 185

### tert-butyl (3-bromonaphthalen-1-yl)methylcarbamate

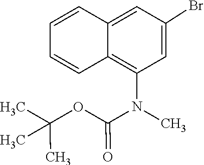 [SMILES: CN(C(=O)OC(C)(C)C)c1cc(Br)cc2ccccc12]

To a solution of tert-butyl (3-bromonaphthalen-1-yl)carbamate (3.04 g, 9.44 mmol) in N,N-dimethylformamide (30 ml) was added sodium hydride (415 mg, 60% purity, 10.4 mmol) at 0° C. After stirring for 10 minutes, iodomethane (1.8 ml, 28 mmol) was added in a portion. After stirring at 20° C. for 16 hours, the reaction mixture was poured into ice water and extracted with ethyl acetate. The organic phase was washed with brine, dried over anhydrous sodium sulfate, filtered and the filtrate was concentrated in vacuum to give a residue. The residue was purified by column chromatography on silica gel (200-300 mesh, petroleum ether:ethyl acetate=1:0 to 10:1) to give tert-butyl (3-bromo-1-naphthyl)methylcarbamate (3.17 g, 99% yield) as yellow oil.

¹H NMR (400 MHz, DMSO-d₆): δ [ppm]=8.18 (s, 1H), 7.97 (d, 1H), 7.74-7.68 (m, 1H), 7.66-7.57 (m, 3H), 3.24 (s, 3H), 1.43-0.95 (m, 9H).

Intermediate 186

### tert-butyl (3-formylnaphthalen-1-yl)methylcarbamate

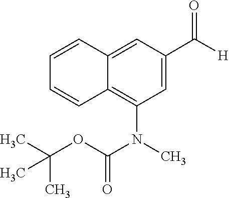 [SMILES: CN(C(=O)OC(C)(C)C)c1cc(C=O)cc2ccccc12]

To a solution of tert-butyl (3-bromonaphthalen-1-yl)methylcarbamate (3.04 g, 9.04 mmol) in tetrahydrofuran (50 ml) was added n-butyllithium (5.4 ml, 2.5 M, 14 mmol) dropwise at −50° C.

After stirring at −50° C. for 10 minutes, a solution of N,N-dimethylformamide (1 ml) in tetrahydrofuran (10 ml) was added dropwise. After stirring at 25° C. for 3 hours, the reaction mixture was poured into water and extracted with ethyl acetate. The combined organic layers were washed with brine, dried over anhydrous sodium sulfate, filtered and the filtrate was concentrated in vacuum to give a residue. The residue was purified by column chromatography on silica gel (200-300 mesh, petroleum ether:ethyl acetate=1:0 to 10:1) to give tert-butyl (3-formyl-1-naphthyl)methylcarbamate (1.50 g, 58% yield) as yellow oil.

¹H NMR (400 MHz, DMSO-d₆): δ [ppm]=10.15 (s, 1H), 8.57 (s, 1H), 8.23 (d, 1H), 7.85-7.67 (m, 4H), 3.27 (s, 3H), 1.49-1.10 (m, 9H).

Intermediate 187

### methyl (2Z)-2-{[(benzyloxy)carbonyl]amino}-3-{4-[(tert-butoxycarbonyl)(methyl)amino]naphthalen-2-yl}prop-2-enoate

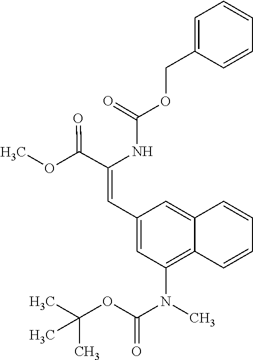 [SMILES: COC(=O)/C(=C/c1cc(N(C)C(=O)OC(C)(C)C)c2ccccc2c1)NC(=O)OCc1ccccc1]

To a solution of methyl {[(benzyloxy)carbonyl]amino}(dimethoxyphosphoryl)acetate (2.09 g, 6.31 mmol) in dichloromethane (60 ml) was added 1,8-diazabicyclo(5.4.0)undec-7-ene (0.940 ml, 6.3 mmol) in a portion. After stirring at 0° C. for 10 minutes, a solution of tert-butyl (3-formylnaphthalen-1-yl)methylcarbamate (1.50 g, 5.26 mmol) in dichloromethane (90 ml) was added. After stirring at 0° C. for 2 hours, the reaction mixture was concentrated under reduced pressure to give a residue. The residue was diluted with ethyl acetate (40 ml) and washed with hydrochloric acid (1M) and brine. The organic phase was dried over anhydrous sodium sulfate and filtered. The filtrate was concentrated in vacuum and purified by column chromatography on silica gel (1000 mesh, petroleum ether:ethyl acetate=50:1 to 3:1) to give methyl {[(benzyloxy)carbonyl]amino}(dimethoxyphosphoryl)acetate (1.65 g, 64% yield) as yellow oil.

LC-MS (Method C): Rₜ=1.029 min; MS (M-100+1): m/z=391.2 ¹H NMR (400 MHz, DMSO-d₆): δ [ppm]=9.36 (br.s, 1H), 8.16 (s, 1H), 7.92 (d, 1H), 7.51-7.83 (m, 4H), 7.04-7.51 (m, 6H), 4.76-5.31 (m, 2H), 3.51-3.87 (m, 3H), 3.21 (s, 3H), 1.16-1.69 (m, 9H).

Intermediate 188

### methyl (2R)-2-{[(benzyloxy)carbonyl]amino}-3-{4-[(tert-butoxycarbonyl)(methyl)amino]naphthalen-2-yl}propanoate

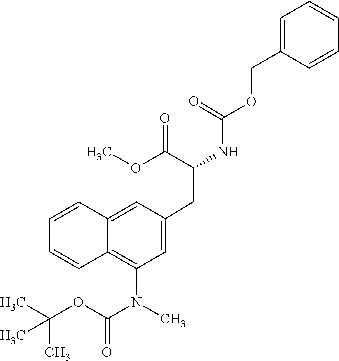 [SMILES: COC(=O)[C@@H](Cc1cc(N(C)C(=O)OC(C)(C)C)c2ccccc2c1)NC(=O)OCc1ccccc1]

To a solution of methyl (2Z)-2-{[(benzyloxy)carbonyl]amino}-3-{4-[(tert-butoxycarbonyl)(methyl) amino]naphthalen-2-yl}prop-2-enoate (1.00 g, 2.04 mmol) in methanol (60 ml) were added (R)—[Rh(COD)(MaxPhos)]BF₄ (72.5 mg, 0.122 mmol). After stirring at room temperature for 16 hours under hydrogen atmosphere (50 psi), the reaction mixture was concentrated and purified by column chromatography on silica gel (200-300 mesh, petroleum ether:ethyl acetate=1:0 to 1:1) to give methyl (2S)-2-{[(benzyloxy)carbonyl]amino}-3-{4-[(tert-butoxycarbonyl)(methyl)amino]naphthalen-2-yl}propanoate (850 mg, 95% purity, 80% yield) as a yellow solid.

¹H NMR (400 MHz, DMSO-d₆): δ [ppm]=7.88 (d, 2H), 7.75-7.64 (m, 2H), 7.58-7.47 (m, 2H), 7.40-7.14 (m, 6H), 4.95 (s, 2H), 4.47-4.35 (m, 1H), 3.72-3.56 (m, 3H), 3.25-3.20 (m, 1H), 3.17 (s, 3H), 3.11-2.96 (m, 1H), 1.50-1.14 (m, 9H).

Intermediate 189

### (2R)-2-{[(benzyloxy)carbonyl]amino}-3-{4-[(tert-butoxycarbonyl)(methyl)amino]naphthalen-2-yl}propanoic acid

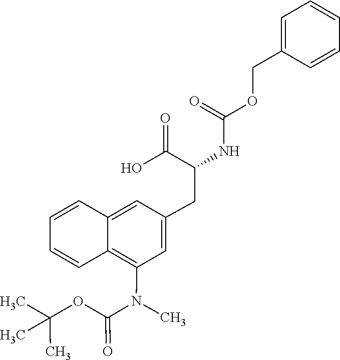 [SMILES: CN(C(=O)OC(C)(C)C)c1cc(C[C@@H](NC(=O)OCc2ccccc2)C(=O)O)cc2ccccc12]

To a solution of methyl (2R)-2-{[(benzyloxy)carbonyl]amino}-3-{4-[(tert-butoxycarbonyl)(methyl)amino]naphthalen-2-yl}propanoate (1.10 g, 95% purity, 2.12 mmol) in tetrahydrofuran (5.7 ml) was added lithium hydroxide (1.3 ml, 2M in water, 2.5 mmol) at room temperature. After stirring at 25° C. for 16 hours, the reaction mixture was concentrated and purified by preparative HPLC (Instrument: ACSWH-GX-Q; Column: Shim-pack C18 150*25*10 μm; eluent A: water (0.225% formic acid), eluent B: acetonitrile; gradient: 0-8 min 55-75% B; flow 25 ml/min; temperature: RT; Detector: UV 220/254 nm.) to give (2S)-2-{[(benzyloxy)carbonyl]amino}-3-(isoquinolin-6-yl)propanoic acid as a yellow solid. The product was dissolved in a mixed solvent of acetonitrile and water, lyophilized to give (2S)-2-{[(benzyloxy)carbonyl]amino}-3-(isoquinolin-6-yl)propanoic acid (830 mg, 99% purity, 81% yield) as a yellow solid.

LC-MS (Method D): Rₜ=0.880 min; MS (M-55): m/z=423.1

¹H NMR (400 MHz, DMSO-d₆): δ [ppm]=12.8 (s, 1H), 7.95-7.80 (m, 1H), 7.80-7.58 (m, 3H), 7.58-7.45 (m, 2H), 7.36 (s, 1H), 7.32-7.04 (m, 5H), 4.94 (s, 2H), 4.42-4.22 (m, 1H), 3.25-3.22 (m, 1H), 3.17 (s, 3H), 3.07-2.92 (m, 1H), 1.60-1.00 (m, 9H).

Intermediate 190

### tri-tert-butyl (5R,12S,16S)-5-({4-[(tert-butoxycarbonyl)(methyl)amino]naphthalen-2-yl}methyl)-3,6,14-trioxo-1-phenyl-2-oxa-4,7,13,15-tetraazaoctadecane-12,16,18-tricarboxylate

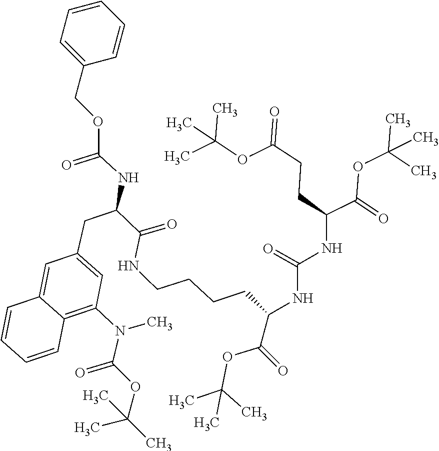 [SMILES: CN(C(=O)OC(C)(C)C)c1cc(C[C@@H](NC(=O)OCc2ccccc2)C(=O)NCCCC[C@H](NC(=O)N[C@@H](CCC(=O)OC(C)(C)C)C(=O)OC(C)(C)C)C(=O)OC(C)(C)C)cc2ccccc12]

di-tert-butyl N-{[(2R)-6-amino-1-tert-butoxy-1-oxohexan-2-yl]carbamoyl}-L-glutamate (1.11 g, 2.28 mmol) and (2S)-2-{[(benzyloxy)carbonyl]amino}-3-{4-[(tert-butoxycarbonyl)(methyl)amino]naphthalen-2-yl}propanoic acid (1.09 g, 2.28 mmol) were solubilised in DMF (14 ml), 4-methylmorpholine (630 μl, 5.7 mmol, CAS-RN: 109-02-4) and HATU (1.21 g, 3.19 mmol) were added and the mixture was stirred under argon overnight at rt. The mixture was diluted with DCM/propan-2-ol, washed with water and brine, dried, evaporated and purified by preparative HPLC (C18, acetonitrile/water with 0.1% formic acid) to give 380 mg (99% purity, 17% yield) of the target compound.

LC-MS (Method 1): Rt=1.62 min; MS (ESIpos): m/z=949 [M+H]⁺

Intermediate 191

### di-tert-butyl (2S)-2-({[(2S)-6-{[(2R)-2-amino-3-{4-[(tert-butoxycarbonyl)(methyl)amino]naphthalen-2-yl}propanoyl]amino}-1-tert-butoxy-1-oxohexan-2-yl]carbamoyl}amino)pentanedioate

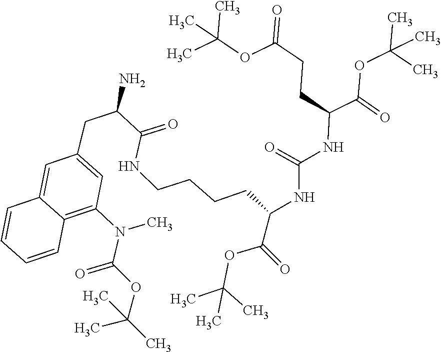 [SMILES: CN(C(=O)OC(C)(C)C)c1cc(C[C@@H](N)C(=O)NCCCC[C@H](NC(=O)N[C@@H](CCC(=O)OC(C)(C)C)C(=O)OC(C)(C)C)C(=O)OC(C)(C)C)cc2ccccc12]

tri-tert-butyl (5R,12S,16S)-5-({4-[(tert-butoxycarbonyl)(methyl)amino]naphthalen-2-yl}methyl)-3,6,14-trioxo-1-phenyl-2-oxa-4,7,13,15-tetraazaoctadecane-12,16,18-tricarboxylate (200 mg, 211 μmol) was solubilised in MeOH (5 ml), Palladium on carbon (2.24 mg, 10% purity, 21.1 μmol) was added and the mixture was purged with hydrogen. The mixture was stirred for 2 h at rt under hydrogen atmosphere. The mixture was filtered over Celite, washed with MeOH and evaporated to give 165 mg (99% purity, 95% yield) of the target compound.

LC-MS (Method 1): Rt=1.25 min; MS (ESIpos): m/z=815 [M+H]⁺

Intermediate 192

### tri-tert-butyl (3R,10S,14S)-1-[(1r,4S)-4-({[(benzyloxy)carbonyl]amino}methyl)cyclohexyl]-3-({4-[(tert-butoxycarbonyl)(methyl)amino]naphthalen-2-yl}methyl)-1,4,12-trioxo-2,5,11,13-tetraazahexadecane-10,14,16-tricarboxylate

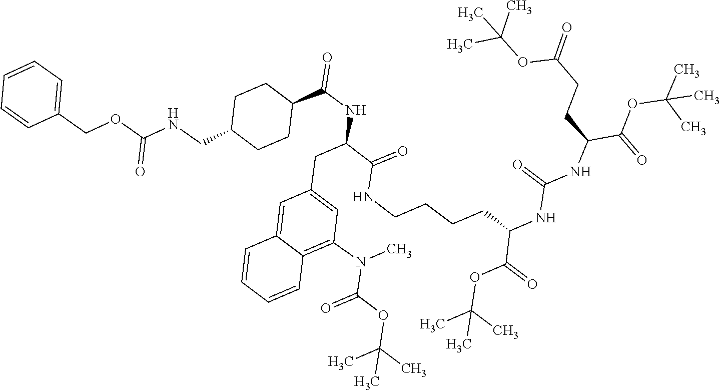 [SMILES: CN(C(=O)OC(C)(C)C)c1cc(C[C@@H](NC(=O)[C@H]2CC[C@H](CNC(=O)OCc3ccccc3)CC2)C(=O)NCCCC[C@H](NC(=O)N[C@@H](CCC(=O)OC(C)(C)C)C(=O)OC(C)(C)C)C(=O)OC(C)(C)C)cc2ccccc12]

di-tert-butyl (2S)-2-({[(2S)-6-{[(2R)-2-amino-3-{4-[(tert-butoxycarbonyl)(methyl)amino]naphthalen-2-yl}propanoyl]amino}-1-tert-butoxy-1-oxohexan-2-yl]carbamoyl}amino)pentanedioate (530 mg, 651 μmol) and (1r,4r)-4-({[(benzyloxy)carbonyl]amino}methyl)cyclohexane-1-carboxylic acid (209 mg, 716 μmol) were solubilised in DMF (10 ml), 4-methylmorpholine (180 μl, 1.6 mmol, CAS-RN: 109-02-4) and HATU (297 mg, 781 μmol) were added and the mixture was stirred under argon overnight at rt. The mixture was filtered and purified by preparative HPLC (C18, acetonitrile/water with 0.1% formic acid) to give 560 mg (95% purity, 75% yield) of the target compound.

LC-MS (Method 1): Rₜ=1.60 min; MS (ESIpos): m/z=1088 [M+H]⁺

¹H-NMR (400 MHz, DMSO-d6) δ[ppm]: 1.139 (0.89), 1.380 (16.00), 1.389 (11.45), 2.518 (1.32), 2.523 (0.82), 2.729 (0.41), 2.888 (0.50), 3.203 (0.80), 4.983 (0.79), 4.986 (0.80), 7.316 (0.49), 7.318 (0.49), 7.329 (1.18), 7.337 (1.01), 7.355 (0.41), 8.165 (0.44).

Intermediate 193

### tri-tert-butyl (3R,10S,14S)-1-[(1r,4S)-4-(aminomethyl)cyclohexyl]-3-({4-[(tert-butoxycarbonyl)(methyl)amino]naphthalen-2-yl}methyl)-1,4,12-trioxo-2,5,11,13-tetraazahexadecane-10,14,16-tricarboxylate

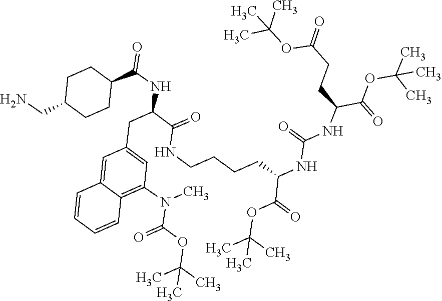 [SMILES: CN(C(=O)OC(C)(C)C)c1cc(C[C@@H](NC(=O)[C@H]2CC[C@H](CN)CC2)C(=O)NCCCC[C@H](NC(=O)N[C@@H](CCC(=O)OC(C)(C)C)C(=O)OC(C)(C)C)C(=O)OC(C)(C)C)cc2ccccc12]

tri-tert-butyl (3R,10S,14S)-1-[(1r,4S)-4-({[(benzyloxy)carbonyl]amino}methyl)cyclohexyl]-3-({4-[(tert-butoxycarbonyl)(methyl)amino]naphthalen-2-yl}methyl)-1,4,12-trioxo-2,5,11,13-tetraazahexadecane-10,14,16-tricarboxylate (560 mg, 515 μmol) was solubilised in MeOH (5 ml), Palladium on carbon (54.8 mg, 10% purity, 51.5 μmol) was added and the mixture was purged with hydrogen. The mixture was stirred for 3 h at rt under hydrogen atmosphere. The mixture was filtered over Celite, washed with MeOH and evaporated. The residue was purified by preparative HPLC (C18, acetonitrile/water with 0.1% formic acid) to give 55.0 mg (100% purity, 11% yield) of the target compound.

LC-MS (Method 1): Rt=1.29 min; MS (ESIpos): m/z=954 [M+H]⁺

### Example 37-A

### (3R,10S,14S)-1-[(1r,4S)-4-(aminomethyl)cyclohexyl]-3-{[4-(methylamino)naphthalen-2-yl]methyl}-1,4,12-trioxo-2,5,11,13-tetraazahexadecane-10,14,16-tricarboxylic acid

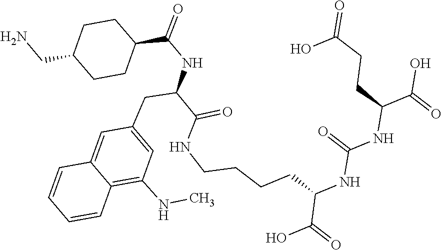 [SMILES: CNc1cc(C[C@@H](NC(=O)[C@H]2CC[C@H](CN)CC2)C(=O)NCCCC[C@H](NC(=O)N[C@@H](CCC(=O)O)C(=O)O)C(=O)O)cc2ccccc12]

tri-tert-butyl (3R,10S,14S)-1-[(1r,4S)-4-(aminomethyl)cyclohexyl]-3-({4-[(tert-butoxycarbonyl)(methyl)amino]naphthalen-2-yl}methyl)-1,4,12-trioxo-2,5,11,13-tetraazahexadecane-10,14,16-tricarboxylate (55.0 mg, 57.7 μmol) was solubilised in DCM (1.9 ml), TFA (890 μl, 12 mmol) was added and the mixture was stirred under argon overnight at rt. The mixture was evaporated and purified by preparative HPLC (C18, acetonitrile/water with 0.1% formic acid) to give 24.0 mg (98% purity, 60% yield) of the target compound.

LC-MS (Method 1): Rt=0.65 min; MS (ESIpos): m/z=686 [M+H]⁺

¹H-NMR (400 MHz, DMSO-d6) δ[ppm]: 0.821 (0.61), 0.852 (1.98), 0.882 (1.83), 0.907 (0.84), 1.092 (0.46), 1.123 (0.99), 1.154 (0.99), 1.176 (0.76), 1.208 (1.22), 1.232 (2.82), 1.271 (2.59), 1.307 (1.37), 1.323 (1.22), 1.343 (1.30), 1.359 (1.30), 1.376 (1.30), 1.394 (1.37), 1.408 (1.68), 1.430 (1.68), 1.444 (1.45), 1.525 (1.22), 1.563 (1.14), 1.605 (1.68), 1.619 (1.98), 1.640 (2.06), 1.681 (1.52), 1.720 (0.99), 1.768 (1.37), 1.791 (1.60), 1.803 (1.68), 1.825 (1.07), 1.840 (0.69), 2.052 (0.76), 2.075 (9.30), 2.081 (1.45), 2.113 (0.69), 2.125 (0.69), 2.139 (0.76), 2.144 (0.76), 2.161 (1.37), 2.174 (1.30), 2.180 (1.30), 2.193 (0.84), 2.224 (0.99), 2.246 (1.52), 2.260 (0.84), 2.268 (0.99), 2.281 (0.91), 2.303 (0.46), 2.336 (1.45), 2.518 (16.00), 2.523 (11.35), 2.574 (2.13), 2.590 (2.82), 2.602 (2.29), 2.619 (0.61), 2.635 (0.46), 2.678 (1.45), 2.805 (1.14), 2.837 (12.11), 2.862 (1.52), 2.888 (0.53), 2.960 (0.76), 2.975 (1.07), 2.994 (2.29), 3.007 (2.59), 3.029 (1.52), 3.042 (1.37), 3.065 (1.22), 3.079 (1.52), 3.096 (1.22), 3.112 (0.99), 3.334 (8.91), 3.913 (1.30), 3.934 (2.59), 3.946 (2.59), 3.965 (1.30), 4.444 (0.84), 4.458 (0.91), 4.467 (1.45), 4.480 (1.45), 4.489 (0.84), 4.502 (0.76), 6.145 (1.07), 6.217 (0.91), 6.335 (5.10), 6.337 (5.18), 6.434 (0.99), 6.450 (0.99), 6.910 (5.18), 7.283 (1.30), 7.286 (1.37), 7.299 (2.13), 7.303 (2.82), 7.320 (2.13), 7.323 (2.06), 7.347 (2.06), 7.350 (2.13), 7.367 (3.05), 7.385 (1.45), 7.610 (3.05), 7.629 (2.74), 7.837 (1.14), 7.993 (2.90), 8.014 (2.67), 8.114 (0.76), 8.221 (1.45).

Intermediate 194 Monomer

### 2,2′-{[(3-{[2-({1-[2-({[(1S,4r)-4-{[(7S,11S,18R)-7,11-bis(tert-butoxycarbonyl)-19-{4-[(tert- butoxycarbonyl)(methyl)amino]naphthalen-2-yl}-2,2-dimethyl-4,9,17-trioxo-3-oxa-8,10,16-triazanonadecan-18-yl]carbamoyl}cyclohexyl]methyl}amino)-2-oxoethyl]-3-hydroxy-6-methyl-2-oxo-1,2-dihydropyridine-4-carbonyl}amino)ethyl](2-{[1-(carboxymethyl)-3-hydroxy-6-methyl-2-oxopyridine-4(2H)-carbonyl]amino}ethyl)amino}propyl)azanediyl]bis[(ethane-2,1-diyl)carbamoyl(3-hydroxy-6-methyl-2-oxopyridine-4,1(2H)-diyl)]}diacetic acid

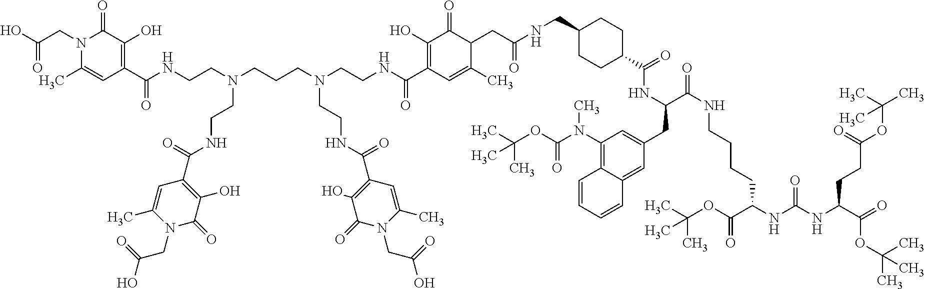 [SMILES: CC1=CC(C(=O)NCCN(CCCN(CCNC(=O)c2cc(C)n(CC(=O)O)c(=O)c2O)CCNC(=O)c2cc(C)n(CC(=O)O)c(=O)c2O)CCNC(=O)c2cc(C)n(CC(=O)O)c(=O)c2O)=C(O)C(=O)C1CC(=O)NC[C@H]1CC[C@H](C(=O)N[C@H](Cc2cc(N(C)C(=O)OC(C)(C)C)c3ccccc3c2)C(=O)NCCCC[C@H](NC(=O)N[C@@H](CCC(=O)OC(C)(C)C)C(=O)OC(C)(C)C)C(=O)OC(C)(C)C)CC1]

2,2′,2″,2′″-(propane-1,3-diylbis{nitrilobis[(ethane-2,1-diyl)carbamoyl(3-hydroxy-6-methyl-2-oxopyridine-4,1(2H)-diyl)]})tetraacetic acid (5.00 mg, 4.62 μmol) and tri-tert-butyl (3R,10S,14S)-1-[(1r,4S)-4-(aminomethyl)cyclohexyl]-3-({4-[(tert-butoxycarbonyl)(methyl)amino]naphthalen-2-yl}methyl)-1,4,12-trioxo-2,5,11,13-tetraazahexadecane-10,14,16-tricarboxylate (6.38 mg, 6.69 μmol) are dissolved in NMP (1.14 mL). DIPEA (4.0 μL, 23 μmol) is added. PyAOP (2.4, 4.6 μmol) in NMP (960 μL) is added. Reaction mix is diluted with 30% ACN/water/0.1% TFA (6 mL) and the products purified by preparative (RP-HPLC using Äkta pure system, Column: Phenomenex Luna 5 μm C18(2) 100 Å, 250×21.2 mm Mobile phase: Water/0.1% TFA; ACN Gradient: 30-80% B over 40 min Flow: 10 mL/min Detection: UV 280/335 nm, tR product: 27 min) affording 1.0 mg of the target compound.

LC-MS (Method K, gradient: 50-100% B over 3 min): Rₜ=0.48 min; MS (ESIpos): m/z=875.6 [M+2H]²⁺

Intermediate 195 Dimer

### 2,2′-{propane-1,3-diylbis[{[2-({1-[2-({[(1S,4r)-4-{[(7S,11S,18R)-7,11-bis(tert-butoxycarbonyl)-19-{4-[(tert-butoxycarbonyl)(methyl)amino]naphthalen-2-yl}-2,2-dimethyl-4,9,17-trioxo-3-oxa-8,10,16-triazanonadecan-18-yl]carbamoyl}cyclohexyl]methyl}amino)-2-oxoethyl]-3-hydroxy-6-methyl-2-oxo-1,2-dihydropyridine-4-carbonyl}amino)ethyl]azanediyl}ethane-2,1-diylcarbamoyl(3-hydroxy-6-methyl-2-oxopyridine-4,1(2H)-diyl)]}diacetic acid

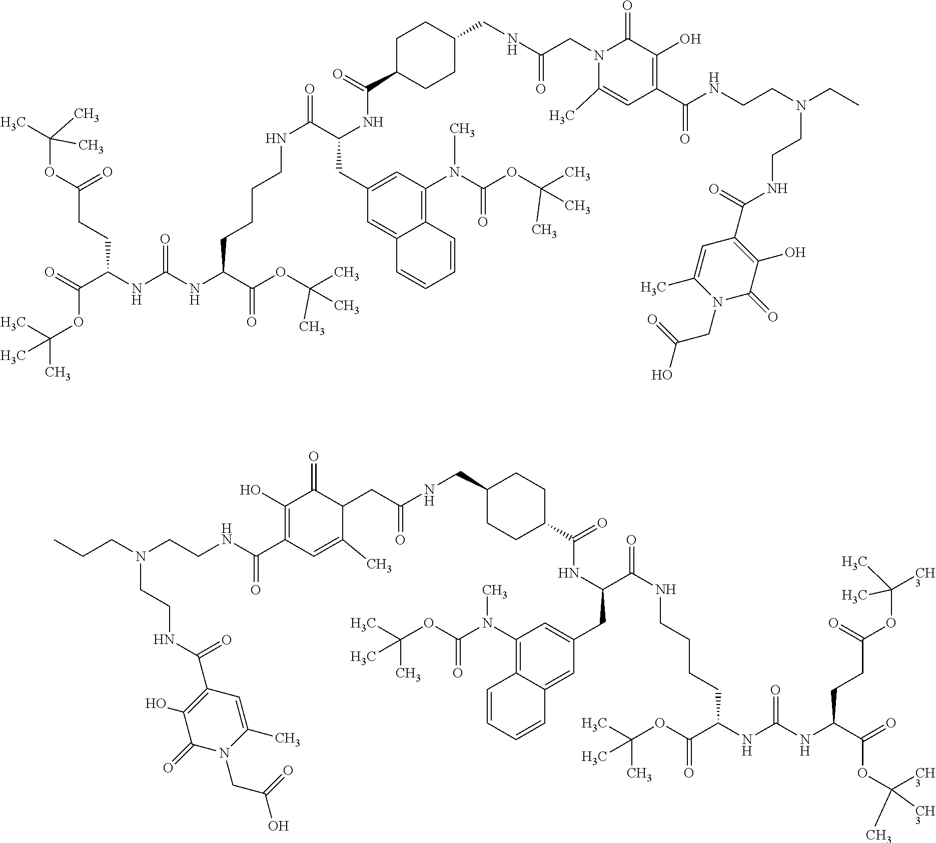

2,2′,2″,2′″-(propane-1,3-diylbis{nitrilobis[(ethane-2,1-diyl)carbamoyl(3-hydroxy-6-methyl-2-oxopyridine-4,1(2H)-diyl)]})tetraacetic acid (5.00 mg, 4.62 μmol) and tri-tert-butyl (3R,10S,14S)-1-[(1r,4S)-4-(aminomethyl)cyclohexyl]-3-({4-[(tert-butoxycarbonyl)(methyl)amino]naphthalen-2-yl}methyl)-1,4,12-trioxo-2,5,11,13-tetraazahexadecane-10,14,16-tricarboxylate (6.38 mg, 6.69 μmol) are dissolved in NMP (1.14 mL). DIPEA (4.0 μL, 23 μmol) is added. PyAOP (2.4, 4.6 μmol) in NMP (960 μL) is added. Reaction mix is diluted with 30% ACN/water/0.1% TFA (6 mL) and the products purified by preparative (RP-HPLC using Äkta pure system, Column: Phenomenex Luna 5 μm C18(2) 100 Å, 250×21.2 mm Mobile phase: Water/0.1% TFA; ACN Gradient: 30-80% B over 40 min Flow: 10 mL/min Detection: UV 280/335 nm, tR product: 37 min) affording 1.6 mg of the target compound.

LC-MS (Method K, gradient: 50-100% B over 3 min): Rₜ=1.42 min; MS (ESIpos): m/z=1477.4 [M+2H]²⁺

Intermediate 196 Trimer

### 2-[4-[2-[3-[bis[2-[[1-[2-[[4-[[(1R)-2-[[(5S)-6-tert-butoxy-5-[[(1R)-4-tert-butoxy-1-tert- butoxycarbonyl-4-oxo-butyl]carbamoylamino]-6-oxo-hexyl]amino]-1-[[4-[tert-butoxycarbonyl(methyl)amino]-2-naphthyl]methyl]-2-oxo-ethyl]carbamoyl]cyclohexyl]methylamino]-2-oxo-ethyl]-3-hydroxy-6-methyl-2-oxo-pyridine-4-carbonyl]amino]ethyl]amino]propyl-[2-[[1-[2-[[4-[[(1R)-2-[[(5S)-6-tert-butoxy-5-[[(1S)-4-tert-butoxy-1-tert-butoxycarbonyl-4-oxo-butyl]carbamoylamino]-6-oxo-hexyl]amino]-1-[[4-[tert-butoxycarbonyl(methyl)amino]-2-naphthyl]methyl]-2-oxo-ethyl]carbamoyl]cyclohexyl]methylamino]-2-oxo-ethyl]-3-hydroxy-6-methyl-2-oxo-pyridine-4-carbonyl]amino]ethyl]amino]ethylcarbamoyl]-3-hydroxy-6-methyl-2-oxo-1-pyridyl]acetic acid

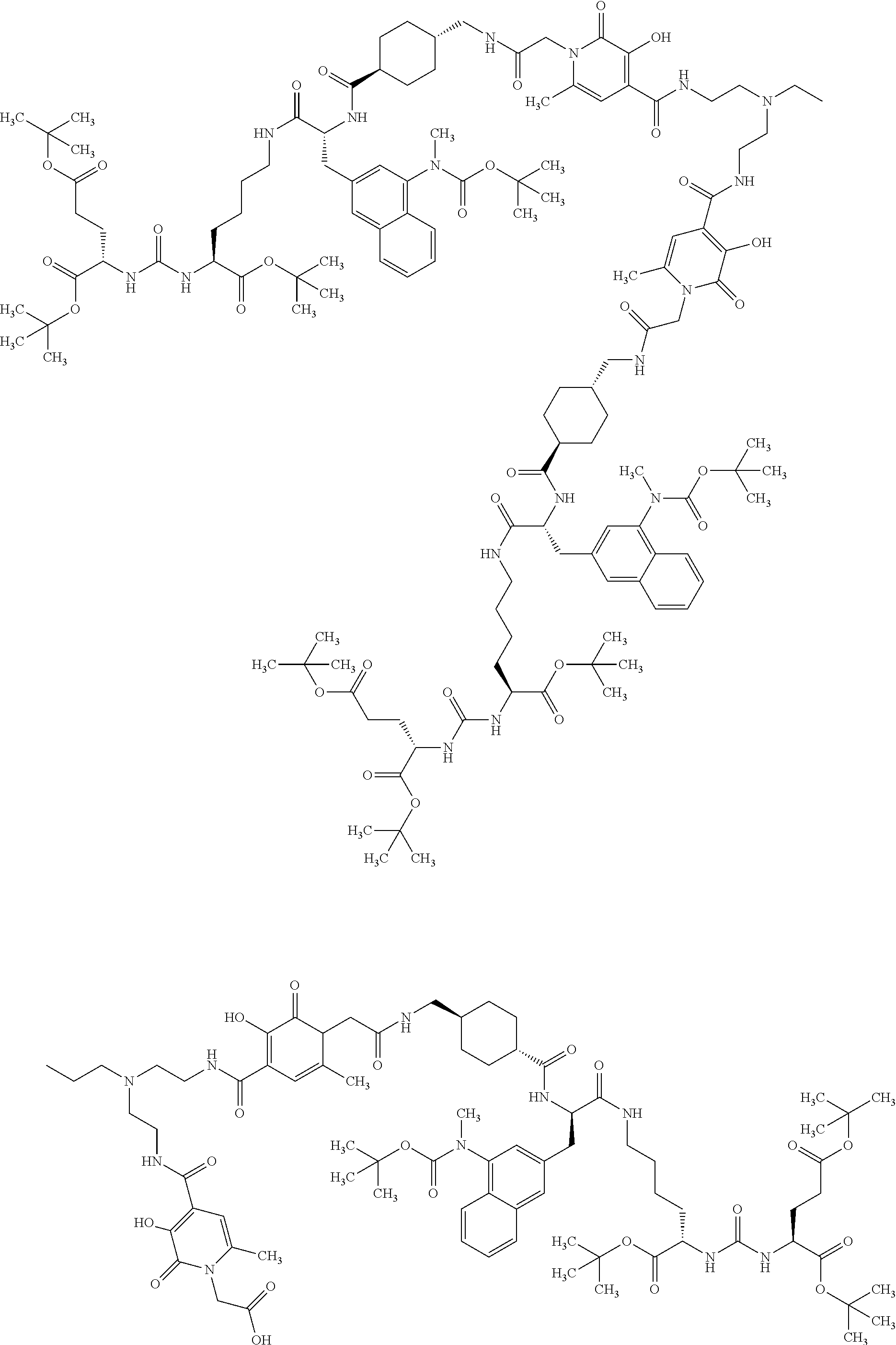

2,2′,2″,2′″-(propane-1,3-diylbis{nitrilobis[(ethane-2,1-diyl)carbamoyl(3-hydroxy-6-methyl-2-oxopyridine-4,1(2H)-diyl)]})tetraacetic acid (5.00 mg, 4.62 μmol) and tri-tert-butyl (3R,10S,14S)-1-[(1r,4S)-4-(aminomethyl)cyclohexyl]-3-({4-[(tert-butoxycarbonyl)(methyl)amino]naphthalen-2-yl}methyl)-1,4,12-trioxo-2,5,11,13-tetraazahexadecane-10,14,16-tricarboxylate (6.38 mg, 6.69 μmol) are dissolved in NMP (1.14 mL). DIPEA (4.0 μL, 23 μmol) is added. PyAOP (2.4, 4.6 μmol) in NMP (960 μL) is added. Reaction mix is diluted with 30% ACN/water/0.1% TFA (6 mL) and the products purified by preparative (RP-HPLC using Äkta pure system, Column: Phenomenex Luna 5 μm C18(2) 100 Å, 250×21.2 mm Mobile phase: Water/0.1% TFA; ACN Gradient: 30-80% B over 40 min Flow: 10 mL/min Detection: UV 280/335 nm, tR product: 47 min) affording 1.4 mg of the target compound.

LC-MS (Method K, gradient: 50-100% B over 3 min): Rₜ=2.23 min; MS (ESIpos): m/z=1944.0 [M+2H]²⁺

### Example 37-C Dimer

### (3R,10S,14S,3′R,10'S,14'S)-1,1′-(propane-1,3-diylbis{[(2-{[1-(carboxymethyl)-3-hydroxy-6-methyl-2-oxo-1,2-dihydropyridine-4-carbonyl]amino}ethyl)azanediyl]ethane-2,1-diylcarbamoyl(3-hydroxy-6-methyl-2-oxopyridine-4,1(2H)-diyl)(1-oxoethane-2,1-diyl)azanediylmethylene(1r,4S)cyclohexane-4,1-diyl})bis(3-{[4-(methylamino)naphthalen-2-yl]methyl}-1,4,12-trioxo-2,5,11,13-tetraazahexadecane-10,14,16-tricarboxylic acid)

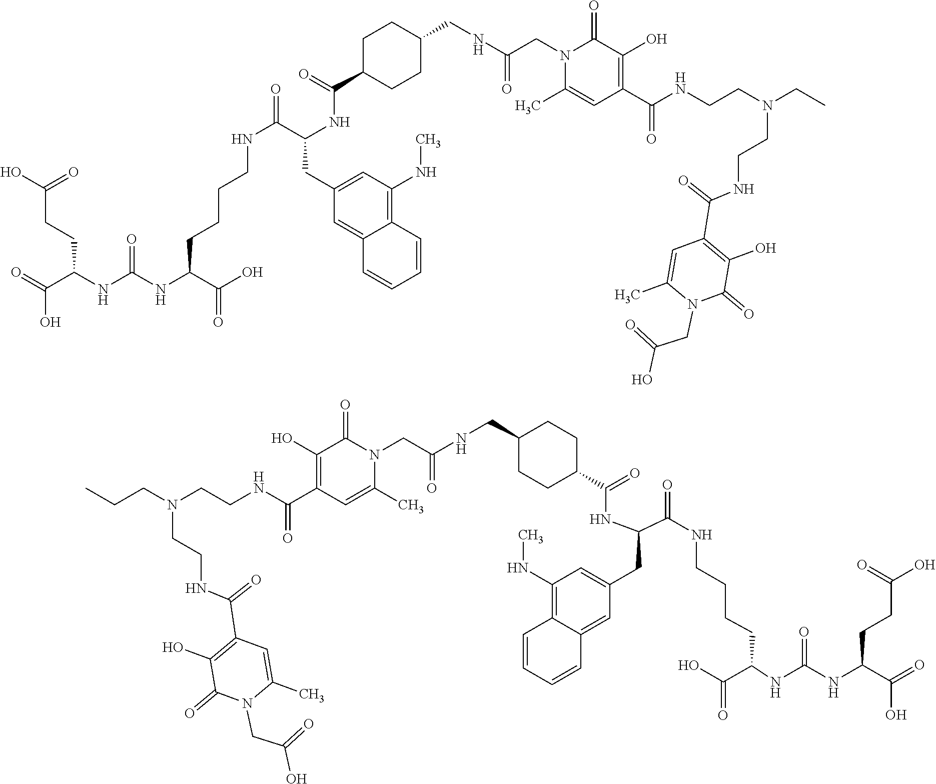

2,2′-{propane-1,3-diylbis[{[2-({1-[2-({[(1S,4r)-4-{[(7S,11S,18R)-7,11-bis(tert-butoxycarbonyl)-19-{4-[(tert-butoxycarbonyl)(methyl)amino]naphthalen-2-yl}-2,2-dimethyl-4,9,17-trioxo-3-oxa-8,10,16-triazanonadecan-18-yl]carbamoyl}cyclohexyl]methyl}amino)-2-oxoethyl]-3-hydroxy-6-methyl-2-oxo-1,2-dihydropyridine-4-carbonyl}amino)ethyl]azanediyl}ethane-2,1-diylcarbamoyl(3-hydroxy-6-methyl-2-oxopyridine-4,1(2H)-diyl)]}diacetic acid (1.60 mg, 0.542 μmol) is treated with 90% TFA in water (0.5 mL). Water (18 mL) is added and the reaction mixture is lyophilised affording 1.60 mg (95% purity, 122% yield) of the target compound.

LC-MS (Method K, gradient: 10-50% B over 3 min): Rₜ=1.56 min; MS (ESIpos): m/z=1208.7 [M+2H]²⁺

### Example 37-C Dimer-²²⁷Th

### (3\hat{ }R,10S,14S,3′R,10'S,14'S)-1,1′-(propane-1,3-diylbis{[(2-{[1-(carboxymethyl)-3-(hydroxy-kappaO)-6-methyl-2-oxo-1,2-dihydropyridine-4-carbonyl]amino}ethyl)azanediyl]ethane-2,1-diylcarbamoyl[3-(hydroxy-kappaO)-6-methyl-2-oxopyridine-4,1(2H)-diyl](1-oxoethane-2,1-diyl)azanediylmethylene(1r,4S)cyclohexane-4,1-diyl})bis(3-{[4-(methylamino)naphthalen-2-yl]methyl}-1,4,12-trioxo-2,5,11,13-tetraazahexadecane-10,14,16-tricarboxylato)(4−)(²²⁷Th)Thorium

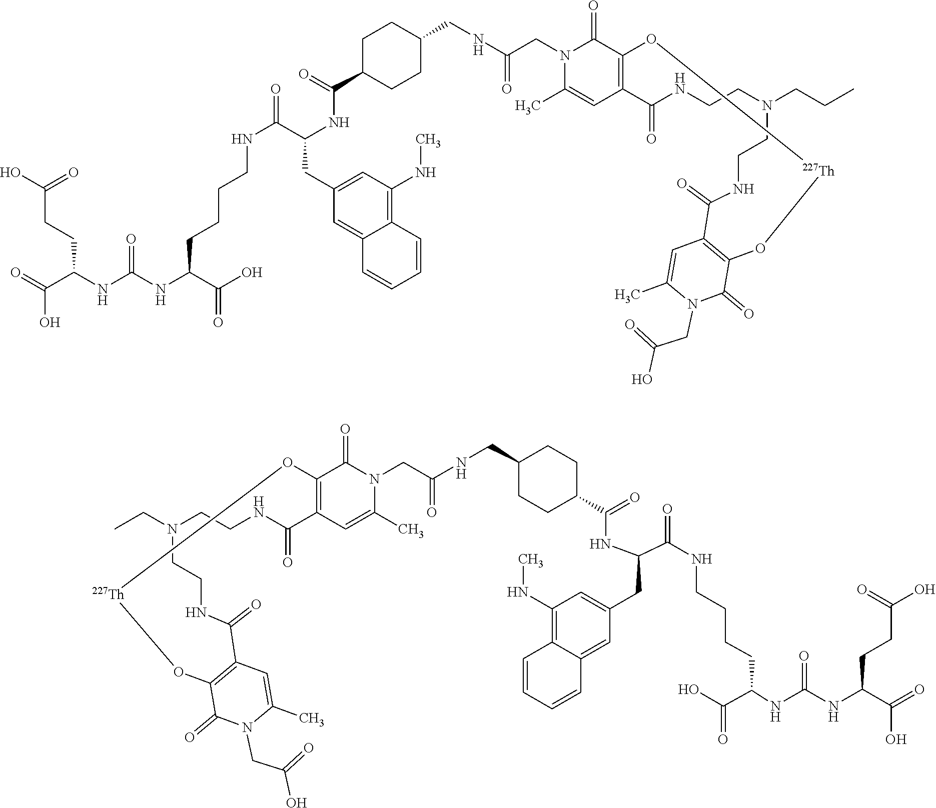

(3R,10S,14S,3′R,10'S,14'S)-1,1-(propane-1,3-diylbis{[(2-{[1-(carboxymethyl)-3-hydroxy-6-methyl-2-oxo-1,2-dihydropyridine-4-carbonyl]amino}ethyl)azanediyl]ethane-2,1-diylcarbamoyl(3-hydroxy-6-methyl-2-oxopyridine-4,1(2H)-diyl)(1-oxoethane-2,1-diyl)azanediylmethylene(1r,4S)cyclohexane-4,1-diyl})bis(3-{[4-(methylamino)naphthalen-2-yl]methyl}-1,4,12-trioxo-2,5,11,13-tetraazahexadecane-10,14,16-tricarboxylic acid) (6.0 μg) dissolved in 153 μL 30 mM citrate buffer (pH 5.5) containing 0.5 mg/mL pABA was mixed with thorium-227 in 0.5 M HCl (2 μL) at 0.3 MBq/nmol specific activity and RAC of 4.5 MBq/mL. 1M carbonate buffer pH 9 (15 μL) was added and mixture incubated for 80 min. The labelling efficiency was determined to be 99% by iTLC.

### Example 37-C Trimer

### (2S)-2-[[(1S)-1-carboxy-5-[[(2R)-2-[[4-[[[2-[4-[2-[2-[[1-[2-[[4-[[(1R)-2-[[(5S)-5-carboxy-5-[[(1S)-1,3-dicarboxypropyl]carbamoylamino]pentyl]amino]-1-[[4-(methylamino)-2-naphthyl]methyl]-2-oxo-ethyl]carbamoyl]cyclohexyl]methylamino]-2-oxo-ethyl]-3-hydroxy-6-methyl-2-oxo-pyridine-4-carbonyl]amino]ethyl-[3-[2-[[1-[2-[[4-[[(1S)-2-[[(5S)-5-carboxy-5-[[(1S)-1,3-dicarboxypropyl]carbamoylamino]pentyl]amino]-1-[[4-(methylamino)-2-naphthyl]methyl]-2-oxo-ethyl]carbamoyl]cyclohexyl]methylamino]-2-oxo-ethyl]-3-hydroxy-6-methyl-2-oxo-pyridine-4-carbonyl]amino]ethyl-[2-[[1-(carboxymethyl)-3-hydroxy-6-methyl-2-oxo-pyridine-4-carbonyl]amino]ethyl]amino]propyl]amino]ethylcarbamoyl]-3-hydroxy-6-methyl-2-oxo-1-pyridyl]acetyl]amino]methyl]cyclohexanecarbonyl]amino]-3-[4-(methylamino)-2-naphthyl]propanoyl]amino]pentyl]carbamoylamino]pentanedioic acid)

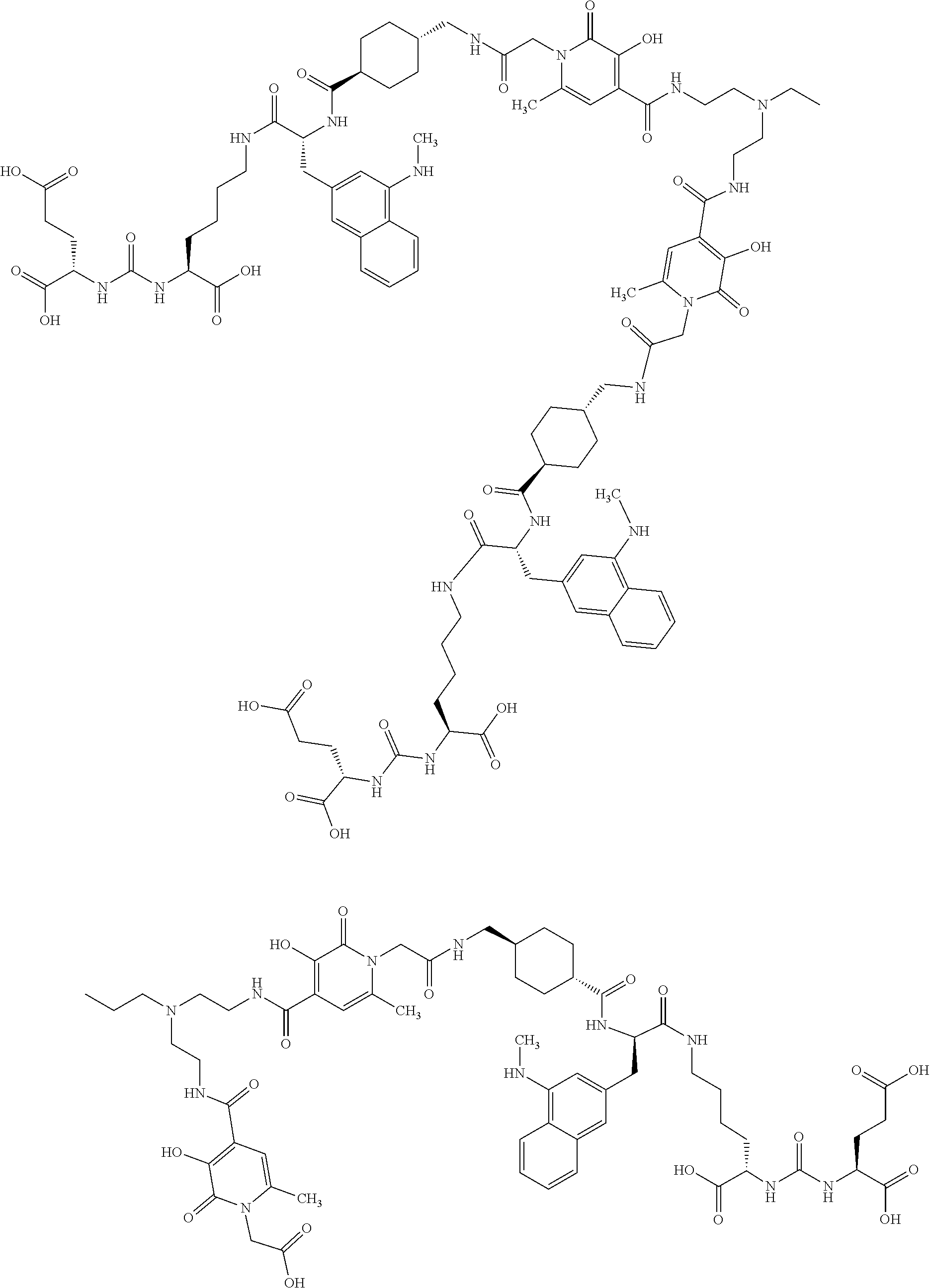

2-[4-[2-[3-[bis[2-[[1-[2-[[4-[[(1R)-2-[[(5S)-6-tert-butoxy-5-[[(1R)-4-tert-butoxy-1-tert- butoxycarbonyl-4-oxo-butyl]carbamoylamino]-6-oxo-hexyl]amino]-1-[[4-[tert-butoxycarbonyl(methyl)amino]-2-naphthyl]methyl]-2-oxo-ethyl]carbamoyl]cyclohexyl]methylamino]-2-oxo-ethyl]-3-hydroxy-6-methyl-2-oxo-pyridine-4-carbonyl]amino]ethyl]amino]propyl-[2-[[1-[2-[[4-[[(1S)-2-[[(5S)-6-tert-butoxy-5-[[(1S)-4-tert-butoxy-1-tert-butoxycarbonyl-4-oxo-butyl]carbamoylamino]-6-oxo-hexyl]amino]-1-[[4-[tert-butoxycarbonyl(methyl)amino]-2-naphthyl]methyl]-2-oxo-ethyl]carbamoyl]cyclohexyl]methylamino]-2-oxo-ethyl]-3-hydroxy-6-methyl-2-oxo-pyridine-4-carbonyl]amino]ethyl]amino]ethylcarbamoyl]-3-hydroxy-6-methyl-2-oxo-1-pyridyl]acetic acid (1.40 mg, 0.36 μmol) is treated with 90% TFA in water (0.5 mL). Water (18 mL) is added and the reaction mixture is lyophilised affording 1.5 mg (135% yield) of the target compound.

LC-MS (Method K, gradient: 10-50% B over 3 min): Rₜ=1.74 min; MS (ESIpos): m/z=1541.6 [M+2H]²⁺

Intermediate 197

### tri-tert-butyl (3R,10S,14S)-3-({4-[(tert-butoxycarbonyl)(methyl)amino]naphthalen-2-yl}methyl)-1,4,12-trioxo-1-[(1r,4S)-4-({2-[4,7,10-tris(2-tert-butoxy-2-oxoethyl)-1,4,7,10-tetraazacyclododecan-1-yl]acetamido}methyl)cyclohexyl]-2,5,11,13-tetraazahexadecane-10,14,16-tricarboxylate

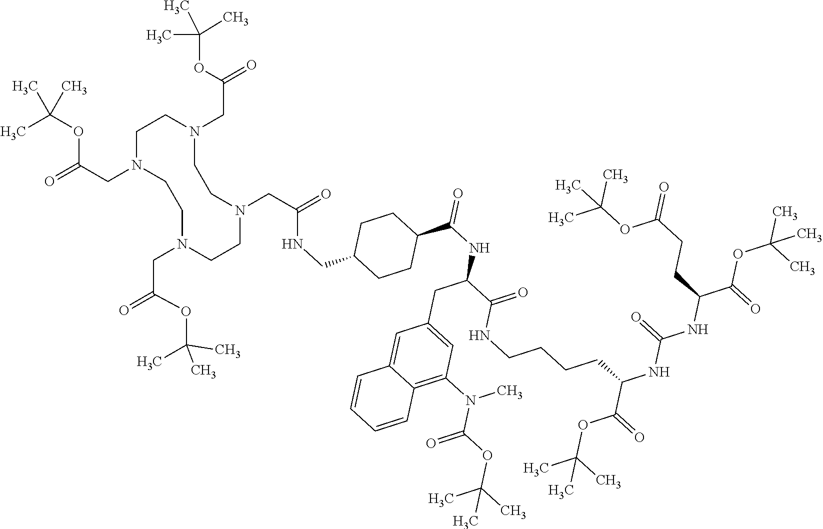 [SMILES: CN(C(=O)OC(C)(C)C)c1cc(C[C@@H](NC(=O)[C@H]2CC[C@H](CNC(=O)CN3CCN(CC(=O)OC(C)(C)C)CCN(CC(=O)OC(C)(C)C)CCN(CC(=O)OC(C)(C)C)CC3)CC2)C(=O)NCCCC[C@H](NC(=O)N[C@@H](CCC(=O)OC(C)(C)C)C(=O)OC(C)(C)C)C(=O)OC(C)(C)C)cc2ccccc12]

[4,7,10-tris(2-tert-butoxy-2-oxoethyl)-1,4,7,10-tetraazacyclododecan-1-yl]acetic acid (228 mg, 399 μmol; CAS-RN:[137076-54-1]), [(1H-benzotriazol-1-yl)oxy](dimethylamino)-N,N-dimethylmethaniminium hexafluoridophosphate(1−) (149 mg, 394 μmol; CAS-RN:94790-37-1) and N,N-diisopropylethylamine (51 μl, 300 μmol) were stirred in DMF (1.5 ml) for 10 min at rt. tri-tert-butyl (3R,10S,14S)-1-[(1r,4S)-4-(aminomethyl)cyclohexyl]-3-({4-[(tert-butoxycarbonyl)(methyl)amino]naphthalen-2-yl}methyl)-1,4,12-trioxo-2,5,11,13-tetraazahexadecane-10,14,16-tricarboxylate (95.0 mg, 99.7 μmol) was added and the mixture was stirred at rt. The mixture was evaporated and purified by preparative HPLC (018, acetonitrile/water with 0.1% formic acid) to give 100 mg (95% purity, 63% yield) of the target compound.

LC-MS (Method 1): Rt=1.56 min; MS (ESIpos): m/z=1508 [M+H]⁺

¹H-NMR (400 MHz, DMSO-d6) δ[ppm]: 1.144 (0.73), 1.232 (0.40), 1.239 (0.60), 1.255 (0.50), 1.269 (0.43), 1.359 (1.57), 1.382 (16.00), 1.390 (10.54), 1.406 (3.66), 1.412 (2.25), 1.480 (2.74), 2.332 (0.55), 2.518 (3.25), 2.523 (2.09), 2.539 (0.62), 2.673 (0.58), 2.909 (0.47), 3.201 (0.55).

### Example 37-B

### (3R,10S,14S)-3-{[4-(methylamino)naphthalen-2-yl]methyl}-1,4,12-trioxo-1-[(1r,4S)-4-({2-[4,7,10-tris(carboxymethyl)-1,4,7,10-tetraazacyclododecan-1-yl]acetamido}methyl)cyclohexyl]-2,5,11,13-tetraazahexadecane-10,14,16-tricarboxylic acid

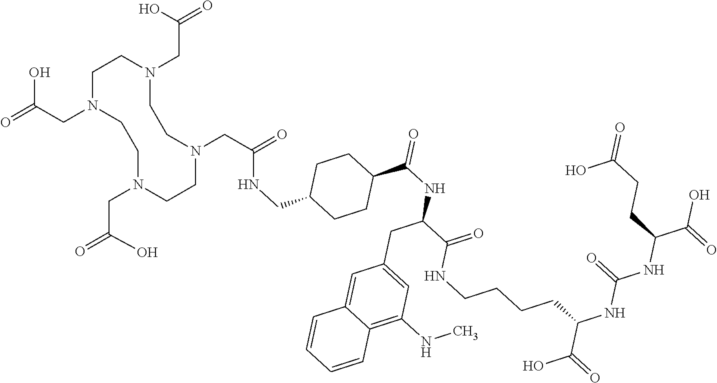 [SMILES: CNc1cc(C[C@@H](NC(=O)[C@H]2CC[C@H](CNC(=O)CN3CCN(CC(=O)O)CCN(CC(=O)O)CCN(CC(=O)O)CC3)CC2)C(=O)NCCCC[C@H](NC(=O)N[C@@H](CCC(=O)O)C(=O)O)C(=O)O)cc2ccccc12]

tri-tert-butyl (3R,10S,14S)-3-({4-[(tert-butoxycarbonyl)(methyl)amino]naphthalen-2-yl}methyl)-1,4,12-trioxo-1-[(1r,4S)-4-({2-[4,7,10-tris(2-tert-butoxy-2-oxoethyl)-1,4,7,10-tetraazacyclododecan-1-yl]acetamido}methyl)cyclohexyl]-2,5,11,13-tetraazahexadecane-10,14,16-tricarboxylate (100 mg, 66.3 μmol) was solubilised in DCM (2.1 ml), TFA (1.0 ml, 13 mmol) was added and the mixture was stirred under argon overnight at rt. The mixture was evaporated and purified by preparative HPLC (C18, acetonitrile/water with 0.1% formic acid) to give 35.0 mg (99% purity, 49% yield) of the target compound.

LC-MS (Method 1): Rt=0.68 min; MS (ESIpos): m/z=1071 [M+H]⁺

¹H-NMR (600 MHz, DMSO-d6) δ[ppm]: 0.000 (6.39), 0.816 (0.95), 0.835 (0.82), 1.116 (0.59), 1.137 (0.59), 1.224 (1.55), 1.236 (2.17), 1.248 (1.94), 1.260 (1.07), 1.288 (0.43), 1.299 (0.64), 1.311 (0.92), 1.323 (1.10), 1.336 (1.17), 1.349 (1.25), 1.361 (1.02), 1.372 (0.67), 1.441 (0.58), 1.454 (0.81), 1.464 (0.95), 1.476 (0.82), 1.489 (0.49), 1.516 (0.92), 1.533 (0.86), 1.595 (1.32), 1.607 (1.35), 1.616 (1.00), 1.642 (0.63), 1.669 (0.94), 1.684 (0.92), 1.699 (1.05), 1.708 (0.97), 1.718 (0.67), 1.723 (0.95), 1.733 (0.66), 1.881 (0.56), 1.893 (0.77), 1.904 (0.77), 1.909 (0.74), 1.917 (0.48), 2.068 (0.71), 2.217 (1.07), 2.227 (1.02), 2.233 (1.33), 2.236 (1.45), 2.242 (1.19), 2.247 (1.35), 2.251 (1.32), 2.262 (1.10), 2.279 (0.43), 2.385 (0.79), 2.388 (1.05), 2.391 (0.77), 2.519 (3.29), 2.522 (3.34), 2.525 (2.75), 2.610 (0.53), 2.613 (0.95), 2.616 (1.22), 2.619 (0.99), 2.622 (0.59), 2.663 (1.66), 2.747 (1.65), 2.825 (1.07), 2.840 (16.00), 2.865 (3.70), 2.905 (2.32), 2.973 (5.19), 2.993 (4.13), 3.006 (4.69), 3.016 (5.60), 3.117 (2.11), 3.173 (0.79), 3.195 (0.79), 3.480 (2.04), 3.500 (2.83), 3.633 (0.64), 3.696 (0.59), 3.975 (0.81), 3.989 (1.37), 3.997 (1.22), 4.002 (0.86), 4.011 (0.64), 4.067 (0.69), 4.076 (1.10), 4.081 (1.27), 4.090 (1.33), 4.094 (0.79), 4.103 (0.58), 4.449 (0.43), 4.463 (0.82), 4.473 (1.04), 4.487 (0.66), 6.316 (1.27), 6.335 (4.95), 6.349 (1.66), 6.905 (3.54), 6.914 (0.59), 7.288 (1.02), 7.290 (1.12), 7.302 (2.07), 7.315 (1.53), 7.355 (1.63), 7.368 (2.16), 7.380 (1.22), 7.615 (2.19), 7.628 (1.93), 7.897 (0.94), 7.970 (0.66), 7.983 (0.64), 7.997 (2.49), 8.011 (2.26), 8.079 (0.76), 8.156 (0.79), 8.318 (0.76).

### Example 37-B 227Th

### (3R,10S,14S)-3-{[4-(methylamino)naphthalen-2-yl]methyl}-1,4,12-trioxo-1-{(1r,4S)-4-[(2-{4,7,10-tris[(carboxy-kappaO)methyl]-1,4,7,10-tetraazacyclododecan-1-yl-kappa²N¹,N⁴}acetamido)methyl]cyclohexyl}-2,5,11,13-tetraazahexadecane-10,14,16-tricarboxylato(3−)(²²⁷Th)Thorium

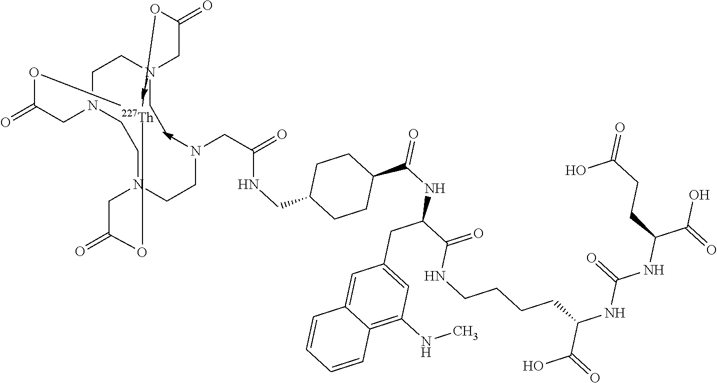

(3R,10S,14S)-3-{[4-(methylamino)naphthalen-2-yl]methyl}-1,4,12-trioxo-1-[(1r,4S)-4-({2-[4,7,10-tris(carboxymethyl)-1,4,7,10-tetraazacyclododecan-1-yl]acetamido}methyl)cyclohexyl]-2,5,11,13-tetraazahexadecane-10,14,16-tricarboxylic acid was dissolved as 10 mM solution in DMSO and diluted to a 250 μM solution in 400 mM sodium acetate buffer (pH 5) containing 0.5 mg/mL pABA. 1.07 μl of this solution was used in a 40 μl labeling reaction. Thorium-227 in 400 mM sodium acetate buffer (pH 5) plus 20% EtOH was added giving a specific activity of 0.375 MBq/nmol and a RAC of 2.5 MBq/mL. The mixture was incubated at 95° C. for 60 min. The labelling efficiency was determined to be 95.5% by iTLC.

#38 Linker

Intermediate 198

### 3,4-dihydro-2H-1-benzopyran

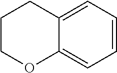

To a solution 2,3-dihydro-4H-1-benzopyran-4-one (30.0 g, 202 mmol) in acetic acid (300 ml) was added palladium on carbon (2.15 g, 20.2 mmol) at room temperature. After stirring at room temperature for 12 hours under hydrogen (50 psi) atmosphere, the reaction mixture was filtered through celite. The filtrate was evaporated under reduced pressure to give chromane (27.0 g, 99% yield) as yellow oil.

¹H NMR (400 MHz, DMSO-d₆) δ [ppm]=7.07-6.99 (m, 2H), 6.79 (td, 1H), 6.74-6.67 (m, 1H), 4.11 (t, 2H), 2.72 (t, 2H), 1.96-1.83 (m, 2H).

Intermediate 199

### 3,4-dihydro-2H-1-benzopyran-6-carbaldehyde

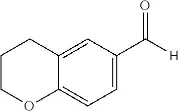 [SMILES: O=Cc1ccc2c(c1)CCCO2]

To a solution of 3,4-dihydro-2H-1-benzopyran (27.0 g, 201 mmol) in 1,2-dichloroethane (270 ml) were added N,N-dimethylformamide (31 ml, 400 mmol) and phosphorus oxychloride (38 ml, 400 mmol) at room temperature. The reaction mixture was heated to 85° C. and stirred at 85° C. for 12 hours. The reaction mixture was quenched with water. The mixture was extracted with ethyl acetate. The organic layer was evaporated under reduced pressure to give a residue. The residue was purified by chromatography (1000 mesh, petroleum ether:ethyl acetate=100:1, then 50:1, then 20:1) to give chromane-6-carbaldehyde (11.5 g, 35% yield) as a colorless oil.

¹H NMR (400 MHz, DMSO-d₆) δ [ppm]=9.80 (s, 1H), 7.67-7.55 (m, 2H), 6.90 (d, 1H), 4.23 (t, 2H), 2.81 (t, 2H), 1.98-1.91 (m, 2H).

Intermediate 200

### methyl (2Z)-2-{[(benzyloxy)carbonyl]amino}-3-(3,4-dihydro-2H-1-benzopyran-6-yl)prop-2-enoate

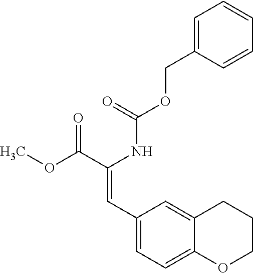 [SMILES: COC(=O)/C(=C/c1ccc2c(c1)CCCO2)NC(=O)OCc1ccccc1]

1,8-Diazabicyclo(5.4.0)undec-7-ene (13 ml, 85 mmol) was added dropwise to a solution of methyl {[(benzyloxy)carbonyl]amino}(dimethoxyphosphoryl)acetate (28.2 g, 85.1 mmol) in dichloromethane (250 ml). After stirring at room temperature for 10 minutes, a solution of 3,4-dihydro-2H-1-benzopyran-6-carbaldehyde (11.5 g, 70.9 mmol) in dichloromethane (100 ml) was added. The reaction was stirred at room temperature for 2 hours. The reaction solvent was removed under reduced pressure to give a residue. The residue was diluted with ethyl acetate (40 ml). The solution was washed with hydrochloric acid (1M) and brine. The organic phase was dried over anhydrous sodium sulfate and filtered. The filtrate was concentrated and purified by column chromatography (1000 mesh, petroleum ether:ethyl acetate=1:0, then 20:1, then 12:1) to give methyl (2Z)-2-{[(benzyloxy)carbonyl]amino}-3-(3,4-dihydro-2H-chromen-6-yl)acrylate (16.9 g, 95% purity, 62% yield) as colorless oil.

LC-MS (Method D): Rₜ=0.848 min; MS (ESIpos): m/z=368.1 [M+H]⁺.

Intermediate 201

### methyl N-[(benzyloxy)carbonyl]-3-(3,4-dihydro-2H-1-benzopyran-6-yl)-D-alaninate

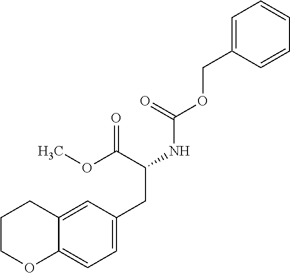 [SMILES: COC(=O)[C@@H](Cc1ccc2c(c1)CCCO2)NC(=O)OCc1ccccc1]

To a solution of methyl (2Z)-2-{[(benzyloxy)carbonyl]amino}-3-(3,4-dihydro-2H-1-benzopyran-6-yl)prop-2-enoate (5.00 g, 13.6 mmol) in methanol (200 ml) were added (R)—[Rh(COD)(MaxPhos)]BF₄ (484 mg, 0.817 mmol). After stirring at room temperature for 16 hours under hydrogen atmosphere (50 psi), the reaction mixture was filtered through silica gel (200-300 mesh). The filtrate was evaporated under reduced pressure to give methyl N-[(benzyloxy)carbonyl]-3-(3,4-dihydro-2H-chromen-6-yl)-L-alaninate (5.10 g, 94% purity, 95% yield) as a yellow oil.

LC-MS (Method D): Rₜ=0.857 min; MS (ESIpos): m/z=370.1 [M+H]⁺.

Intermediate 202

### N-[(benzyloxy)carbonyl]-3-(3,4-dihydro-2H-1-benzopyran-6-yl)-D-alanine

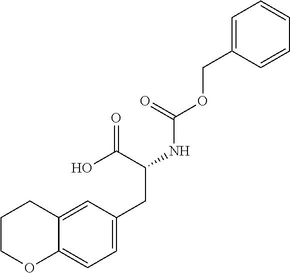 [SMILES: O=C(N[C@H](Cc1ccc2c(c1)CCCO2)C(=O)O)OCc1ccccc1]

To a solution of methyl N-[(benzyloxy)carbonyl]-3-(3,4-dihydro-2H-chromen-6-yl)-D-alaninate (5.10 g, 13.8 mmol) in pyridine (80 ml) was added lithium iodide anhydrous (5.5 g, 41.4 mmol) at room temperature. The reaction mixture was heated to 110° C. and stirred at 110° C. for 24 hours. The reaction mixture was cooled to room temperature and evaporated under reduced pressure to give a residue. The residue was dissolved in methanol. The pH was adjusted to 5-6 through hydrochloric acid (1M). The solution was evaporated under reduced pressure and purified by preparative HPLC (Instrument: Shimadzu LC-20AP; Column: Phenomenex luna C18 250*100 mm*10 μm; eluent A: 0.225% formic acid in water, eluent B: acetonitrile; gradient: 0-30 min 30-60% B; flow 100 ml/min; temperature: room temperature; Detector: UV 220/254 nm) to give N-[(benzyloxy)carbonyl]-3-(3,4-dihydro-2H-chromen-6-yl)-L-alanine (2.42 g, 98% purity, 48% yield) as a yellow solid.

LC-MS (Method D): Rₜ=0.803 min; MS (ESIpos): m/z=356.1 [M+H]⁺.

¹H NMR (400 MHz, DMSO-d₆) δ [ppm]=7.58 (d, 1H), 7.38-7.17 (m, 5H), 6.96-6.88 (m, 2H), 6.61 (d, 1H), 4.98 (s, 2H), 4.15-4.02 (m, 3H), 2.93 (dd, 1H), 2.76-2.70 (m, 1H), 2.68-2.61 (m, 2H), 1.94-1.78 (m, 2H).

Intermediate 203

### tri-tert-butyl (5R,12S,16S)-5-[(3,4-dihydro-2H-1-benzopyran-6-yl)methyl]-3,6,14-trioxo-1-phenyl-2-oxa-4,7,13,15-tetraazaoctadecane-12,16,18-tricarboxylate

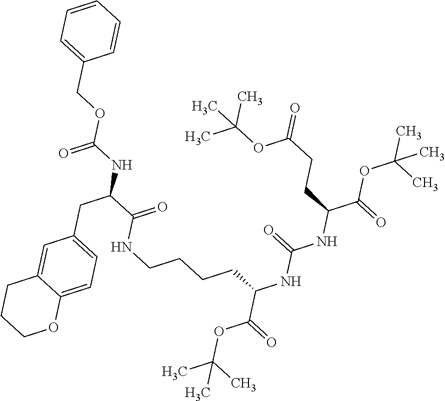 [SMILES: CC(C)(C)OC(=O)CC[C@H](NC(=O)N[C@@H](CCCCNC(=O)[C@@H](Cc1ccc2c(c1)CCCO2)NC(=O)OCc1ccccc1)C(=O)OC(C)(C)C)C(=O)OC(C)(C)C]

di-tert-butyl N-{[(2R)-6-amino-1-tert-butoxy-1-oxohexan-2-yl]carbamoyl}-L-glutamate (1.11 g, 2.28 mmol) and N-[(benzyloxy)carbonyl]-3-(3,4-dihydro-2H-1-benzopyran-6-yl)-L-alanine (810 mg, 2.28 mmol) were solubilised in DMF (14 ml), 4-methylmorpholine (630 μl, 5.7 mmol, CAS-RN: 109-02-4) and HATU (1.21 g, 3.19 mmol) were added and the mixture was stirred under argon overnight at rt. The mixture was diluted with DCM/propan-2-ol, washed with water and brine, dried, evaporated and purified by flash chromatography (SiO2, DCM/Ethanol gradient 0%-5%) and preparative HPLC (C18, acetonitrile/water with 0.1% formic acid) to give 583 mg (95% purity, 29% yield) of the target compound.

LC-MS (Method 1): Rt=1.51 min; MS (ESIpos): m/z=826 [M+H]⁺

Intermediate 204

### di-tert-butyl (2S)-2-({[(2S)-6-{[(2R)-2-amino-3-(3,4-dihydro-2H-1-benzopyran-6-yl)propanoyl]amino}-1-tert-butoxy-1-oxohexan-2-yl]carbamoyl}amino)pentanedioate

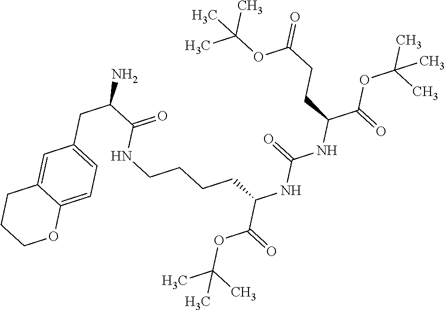 [SMILES: CC(C)(C)OC(=O)CC[C@H](NC(=O)N[C@@H](CCCCNC(=O)[C@H](N)Cc1ccc2c(c1)CCCO2)C(=O)OC(C)(C)C)C(=O)OC(C)(C)C]

tri-tert-butyl (5R,12S,16S)-5-[(3,4-dihydro-2H-1-benzopyran-6-yl)methyl]-3,6,14-trioxo-1-phenyl-2-oxa-4,7,13,15-tetraazaoctadecane-12,16,18-tricarboxylate (580 mg, 703 μmol) was solubilised in MeOH (5.7 ml), Palladium on carbon (74.8 mg, 10% purity, 70.3 μmol) was added and the mixture was purged with hydrogen. The mixture was stirred for 3 h at 40° C. under hydrogen atmosphere. The mixture was filtered over Celite, washed with MeOH and evaporated to give 488 mg (97% purity, 97% yield) of the target compound.

LC-MS (Method 1): Rt=1.09 min; MS (ESIpos): m/z=692 [M+H]⁺

Intermediate 205

### tri-tert-butyl (3R,10S,14S)-1-[(1r,4S)-4-({[(benzyloxy)carbonyl]amino}methyl)cyclohexyl]-3-[(3,4-dihydro-2H-1-benzopyran-6-yl)methyl]-1,4,12-trioxo-2,5,11,13-tetraazahexadecane-10,14,16-tricarboxylate

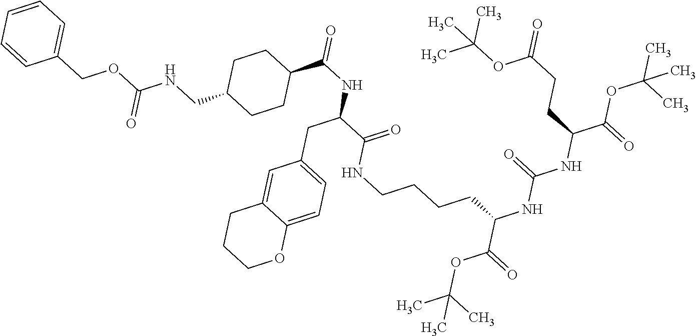 [SMILES: CC(C)(C)OC(=O)CC[C@H](NC(=O)N[C@@H](CCCCNC(=O)[C@@H](Cc1ccc2c(c1)CCCO2)NC(=O)[C@H]1CC[C@H](CNC(=O)OCc2ccccc2)CC1)C(=O)OC(C)(C)C)C(=O)OC(C)(C)C]

(1r,4r)-4-({[(benzyloxy)carbonyl]amino}methyl)cyclohexane-1-carboxylic acid (226 mg, 777 μmol) was solubilised in DMF (11 ml), 4-methylmorpholine (190 μl, 1.8 mmol, CAS-RN: 109-02-4) and HATU (322 mg, 848 μmol) were added, stirred for 20 min at rt, di-tert-butyl (2S)-2-({[(2S)-6-{[(2R)-2-amino-3-(3,4-dihydro-2H-1-benzopyran-6-yl)propanoyl]amino}-1-tert-butoxy-1-oxohexan-2-yl]carbamoyl}amino)pentanedioate (488 mg, 706 μmol) was added and the mixture was stirred under argon overnight at rt. The mixture was filtered and purified by preparative HPLC (C18, acetonitrile/water with 0.1% formic acid) to give 360 mg (75% purity, 40% yield) of the target compound.

LC-MS (Method 1): Rt=1.52 min; MS (ESIpos): m/z=965 [M+H]⁺

¹H-NMR (400 MHz, DMSO-d6) δ[ppm]: 1.385 (16.00), 1.392 (9.79), 1.396 (8.35), 2.522 (0.67), 4.046 (0.43), 4.059 (0.50), 4.996 (1.14), 6.275 (0.52), 6.296 (0.48), 6.849 (0.79), 7.323 (0.41), 7.339 (1.03), 7.344 (1.24).

Intermediate 206

### tri-tert-butyl (3R,10S,14S)-1-[(1r,4S)-4-(aminomethyl)cyclohexyl]-3-[(3,4-dihydro-2H-1-benzopyran-6-yl)methyl]-1,4,12-trioxo-2,5,11,13-tetraazahexadecane-10,14,16-tricarboxylate

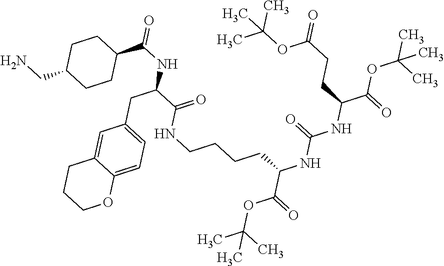 [SMILES: CC(C)(C)OC(=O)CC[C@H](NC(=O)N[C@@H](CCCCNC(=O)[C@@H](Cc1ccc2c(c1)CCCO2)NC(=O)[C@H]1CC[C@H](CN)CC1)C(=O)OC(C)(C)C)C(=O)OC(C)(C)C]

tri-tert-butyl (3R,10S,14S)-1-[(1r,4S)-4-({[(benzyloxy)carbonyl]amino}methyl)cyclohexyl]-3-[(3,4-dihydro-2H-1-benzopyran-6-yl)methyl]-1,4,12-trioxo-2,5,11,13-tetraazahexadecane-10,14,16-tricarboxylate (360 mg, 373 μmol) was solubilised in MeOH (3.0 ml), Palladium on carbon (39.7 mg, 10% purity, 37.3 μmol) was added and the mixture was purged with hydrogen. The mixture was stirred for 2 h at rt under hydrogen atmosphere. The mixture was filtered over Celite, washed with MeOH and evaporated to give 324 mg (93% purity, 97% yield) of the target compound.

LC-MS (Method 1): Rₜ=1.18 min; MS (ESIpos): m/z=831 [M+H]⁺

¹H-NMR (400 MHz, DMSO-d6) δ[ppm]: 1.385 (16.00), 1.394 (8.51), 1.396 (9.15), 2.224 (0.91), 2.326 (0.44), 2.518 (1.02), 2.522 (0.63), 6.276 (0.46), 6.298 (0.43), 6.852 (0.56).

### Example 38-A

### N⁶—{N-[(1r,4S)-4-(aminomethyl)cyclohexane-1-carbonyl]-3-(3,4-dihydro-2H-1-benzopyran-6-yl)-D-alanyl}-N²-{[(1S)-1,3-dicarboxypropyl]carbamoyl}-L-lysine

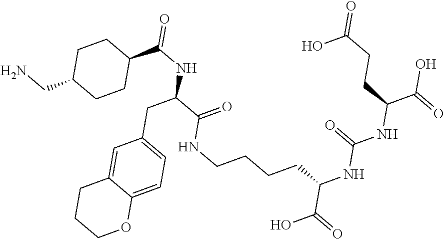 [SMILES: NC[C@H]1CC[C@H](C(=O)N[C@H](Cc2ccc3c(c2)CCCO3)C(=O)NCCCC[C@H](NC(=O)N[C@@H](CCC(=O)O)C(=O)O)C(=O)O)CC1]

tri-tert-butyl (3R,10S,14S)-1-[(1r,4S)-4-(aminomethyl)cyclohexyl]-3-[(3,4-dihydro-2H-1-benzopyran-6-yl)methyl]-1,4,12-trioxo-2,5,11,13-tetraazahexadecane-10,14,16-tricarboxylate (20.0 mg, 24.1 μmol) was solubilised in DCM (780 μl), TFA (930 μl, 12 mmol) was added and the mixture was stirred under argon overnight at rt. The mixture was evaporated and purified by preparative HPLC (C18, acetonitrile/water with 0.1% formic acid) to give 2.00 mg (95% purity, 12% yield) of the target compound.

¹H-NMR (400 MHz, DMSO-d6) δ[ppm]: 0.851 (0.89), 0.889 (1.24), 0.918 (1.03), 1.231 (2.61), 1.248 (2.82), 1.312 (1.51), 1.327 (1.30), 1.342 (1.17), 1.352 (1.17), 1.444 (1.30), 1.561 (1.44), 1.595 (1.99), 1.694 (1.58), 1.799 (1.65), 1.854 (2.20), 1.863 (2.47), 1.879 (2.13), 2.050 (0.62), 2.080 (1.03), 2.109 (1.03), 2.121 (0.82), 2.146 (1.10), 2.164 (1.17), 2.230 (0.82), 2.246 (1.03), 2.270 (0.82), 2.282 (0.69), 2.305 (0.41), 2.318 (1.44), 2.322 (3.02), 2.326 (4.05), 2.331 (3.02), 2.336 (1.37), 2.461 (5.36), 2.518 (16.00), 2.523 (9.82), 2.581 (1.30), 2.598 (2.82), 2.637 (3.02), 2.659 (4.19), 2.664 (4.94), 2.669 (5.91), 2.673 (4.39), 2.791 (1.17), 2.803 (1.37), 2.824 (1.10), 2.837 (0.96), 2.982 (1.10), 3.041 (1.17), 3.336 (4.05), 3.913 (2.06), 4.048 (2.88), 4.061 (3.85), 4.073 (2.75), 4.264 (0.62), 4.286 (1.10), 4.299 (1.17), 4.322 (0.62), 6.111 (0.62), 6.425 (0.55), 6.559 (3.09), 6.581 (3.36), 6.866 (5.22), 6.882 (1.65).

Intermediate 207

### tri-tert-butyl (3R,10S,14S)-3-[(3,4-dihydro-2H-1-benzopyran-6-yl)methyl]-1,4,12-trioxo-1-[(1r,4S)-4-({2-[4,7,10-tris(2-tert-butoxy-2-oxoethyl)-1,4,7,10-tetraazacyclododecan-1-yl]acetamido}methyl)cyclohexyl]-2,5,11,13-tetraazahexadecane-10,14,16-tricarboxylate

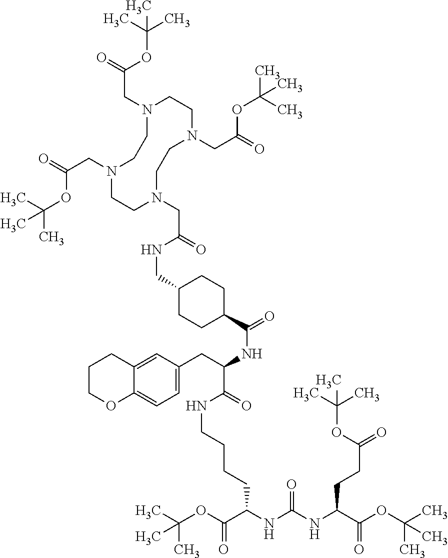 [SMILES: CC(C)(C)OC(=O)CC[C@H](NC(=O)N[C@@H](CCCCNC(=O)[C@@H](Cc1ccc2c(c1)CCCO2)NC(=O)[C@H]1CC[C@H](CNC(=O)CN2CCN(CC(=O)OC(C)(C)C)CCN(CC(=O)OC(C)(C)C)CCN(CC(=O)OC(C)(C)C)CC2)CC1)C(=O)OC(C)(C)C)C(=O)OC(C)(C)C]

[4,7,10-tris(2-tert-butoxy-2-oxoethyl)-1,4,7,10-tetraazacyclododecan-1-yl]acetic acid (190 mg, 332 μmol; CAS-RN:[137076-54-1]), [(1H-benzotriazol-1-yl)oxy](dimethylamino)-N,N-dimethylmethaniminium hexafluoridophosphate(1−) (125 mg, 328 μmol; CAS-RN:94790-37-1) and N,N-diisopropylethylamine (47 μl, 290 μmol) were stirred in DMF (3.2 ml) for 10 min at rt. tri-tert-butyl (3R,10S,14S)-1-[(1r,4S)-4-(aminomethyl)cyclohexyl]-3-[(3,4-dihydro-2H-1-benzopyran-6-yl)methyl]-1,4,12-trioxo-2,5,11,13-tetraazahexadecane-10,14,16-tricarboxylate (69.0 mg, 83.1 μmol) was added and the mixture was stirred overnight at rt. The mixture was evaporated and purified by preparative HPLC (C18, acetonitrile/water with 0.1% formic acid) to give 55.0 mg (90% purity, 43% yield) of the target compound.

¹H-NMR (400 MHz, DMSO-d6) δ[ppm]: 1.385 (16.00), 1.394 (9.98), 1.396 (10.45), 1.405 (3.63), 1.414 (1.34), 1.420 (2.50), 1.484 (2.08), 2.518 (3.44), 2.522 (2.39), 6.274 (0.43), 6.295 (0.40).

### Example 38-B

N⁶-{3-(3,4-dihydro-2H-1-benzopyran-6-yl)-N-[(1r,4S)-4-({2-[4,7,10-tris(carboxymethyl)-1,4,7,10-tetraazacyclododecan-1-yl]acetamido}methyl)cyclohexane-1-carbonyl]-D-alanyl}-N²-{[(1S)-1,3-dicarboxypropyl]carbamoyl}-L-lysine

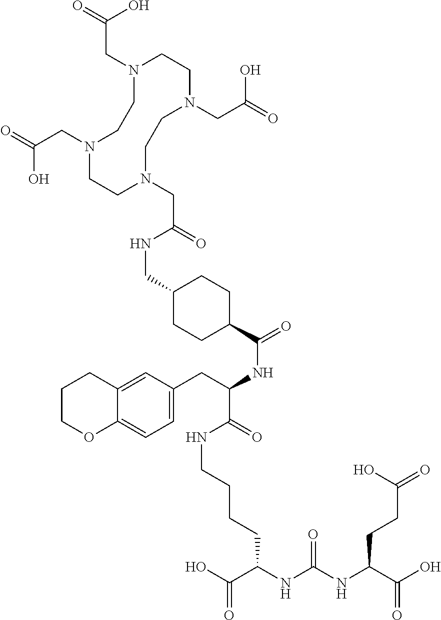 [SMILES: O=C(O)CC[C@H](NC(=O)N[C@@H](CCCCNC(=O)[C@@H](Cc1ccc2c(c1)CCCO2)NC(=O)[C@H]1CC[C@H](CNC(=O)CN2CCN(CC(=O)O)CCN(CC(=O)O)CCN(CC(=O)O)CC2)CC1)C(=O)O)C(=O)O]

tri-tert-butyl (3R,10S,14S)-3-[(3,4-dihydro-2H-1-benzopyran-6-yl)methyl]-1,4,12-trioxo-1-[(1r,4S)-4-({2-[4,7,10-tris(2-tert-butoxy-2-oxoethyl)-1,4,7,10-tetraazacyclododecan-1-yl]acetamido}methyl)cyclohexyl]-2,5,11,13-tetraazahexadecane-10,14,16-tricarboxylate (53.0 mg, 38.3 μmol) was solubilised in DCM (1.2 ml), TFA (590 μl, 7.7 mmol) was added and the mixture was stirred under argon overnight at rt. The mixture was evaporated and purified by preparative HPLC (C18, acetonitrile/water with 0.1% formic acid) to give 11.0 mg (95% purity, 26% yield) of the target compound.

LC-MS (Method 1): Rₜ=0.46 min; MS (ESIneg): m/z=1043 [M−H]⁻

¹H-NMR (400 MHz, DMSO-d6) δ[ppm]: 0.784 (0.76), 0.814 (0.83), 1.228 (1.45), 1.319 (1.18), 1.352 (1.18), 1.463 (0.90), 1.594 (1.25), 1.722 (0.62), 1.738 (0.48), 1.907 (2.08), 2.032 (0.69), 2.209 (0.76), 2.224 (1.25), 2.246 (1.25), 2.263 (0.69), 2.318 (1.45), 2.322 (3.05), 2.326 (4.02), 2.331 (3.05), 2.336 (1.52), 2.518 (16.00), 2.522 (9.77), 2.659 (2.49), 2.664 (3.88), 2.669 (4.71), 2.673 (3.53), 2.678 (1.87), 2.729 (1.59), 2.889 (2.84), 2.979 (4.09), 3.095 (1.66), 3.152 (1.66), 3.185 (1.80), 3.436 (6.03), 3.987 (0.69), 4.000 (0.69), 4.073 (0.76), 4.086 (0.69), 4.534 (0.62), 6.298 (0.97), 6.317 (1.25), 6.333 (0.83), 7.555 (1.18), 7.576 (1.25), 7.699 (1.66), 7.714 (1.73), 7.731 (2.08), 7.993 (2.08), 8.015 (1.66), 8.075 (1.11), 8.136 (0.90), 8.437 (3.19), 8.451 (2.70), 9.233 (3.74).

### Example 38-B 227Th

### {N⁶-[3-(3,4-dihydro-2H-1-benzopyran-6-yl)-N-{(1r,4S)-4-[(2-{4,7,10-tris[(carboxy-kappaO)methyl]-1,4,7,10-tetraazacyclododecan-1-yl-kappa²N¹,N⁴}acetamido)methyl]cyclohexane-1-carbonyl}-D-alanyl]-N²-{[(1S)-1,3-dicarboxypropyl]carbamoyl}-L-lysinato(3−)}(²²⁷Th)Thorium

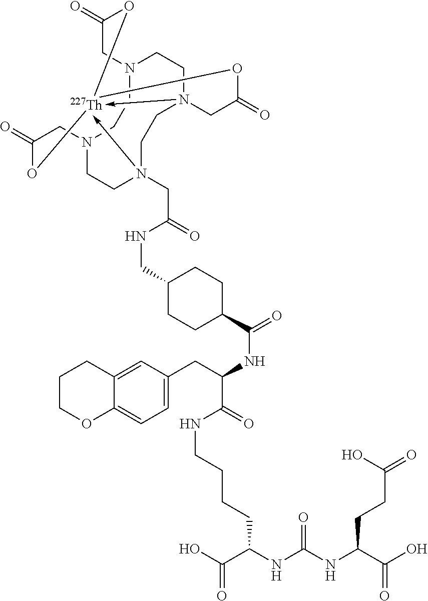

Xxx

N⁶-{3-(3,4-dihydro-2H-1-benzopyran-6-yl)-N-[(1r,4S)-4-({2-[4,7,10-tris(carboxymethyl)-1,4,7,10-tetraazacyclododecan-1-yl]acetamido}methyl)cyclohexane-1-carbonyl]-D-alanyl}-N²-{[(1S)-1,3-dicarboxypropyl]carbamoyl}-L-lysine was dissolved as 10 mM solution in DMSO and diluted to a 250 μM solution in 400 mM sodium acetate buffer (pH 5) containing 0.5 mg/mL pABA. 1.07 μl of this solution was used in a 40 μl labeling reaction. Thorium-227 in 400 mM sodium acetate buffer (pH 5) plus 20% EtOH was added giving a specific activity of 0.375 MBq/nmol and a RAC of 2.5 MBq/mL. The mixture was incubated at 95° C. for 60 min. The labelling efficiency was determined to be 79.9% by iTLC.

#39 Linker

Intermediate 208

### methyl 3-aminonaphthalene-2-carboxylate

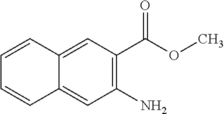

To a solution of 3-amino-2-naphthoic acid (10.0 g, 53.4 mmol) in methanol (100 ml) was added thionyl chloride (11.69 ml, 160.3 mmol) dropwise at 0° C. After refluxing for 16 hours, the reaction mixture was concentrated and dissolved in ethyl acetate. The solution was washed with saturated sodium bicarbonate. The organic phase was dried over anhydrous sodium sulfate and filtered. The filtrate was concentrated under reduced pressure and purified by column chromatography (1000 mesh, petroleum ether:ethyl acetate=10:1, then 5:1, then 3:1) to give methyl 3-amino-2-naphthoate (5 g, 46% yield) as a yellow solid.

¹H NMR (400 MHz, CDCl₃) δ [ppm]=8.51 (s, 1H), 7.72 (d, 1H), 7.54 (d, 1H), 7.43 (t, 1H), 7.21 (t, 1H), 7.00 (s, 1H), 3.98 (s, 3H).

Intermediate 209

### methyl 3-[(tert-butoxycarbonyl)amino]naphthalene-2-carboxylate

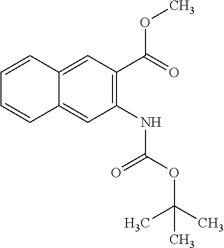 [SMILES: COC(=O)c1cc2ccccc2cc1NC(=O)OC(C)(C)C]

A mixture of methyl 3-aminonaphthalene-2-carboxylate (5.00 g, 99% purity, 24.6 mmol) in di-tert-butyl dicarbonate (10 ml, 44 mmol) was stirred at 60° C. for 16 hours. The reaction mixture was triturated with petroleum ether. The suspension was filtered. The solid cake was washed with petroleum ether and dried under reduced pressure to give methyl 3-[(tert-butoxycarbonyl)amino]-2-naphthoate (5.60 g, 90% purity, 68% yield) as a white solid.

¹H NMR (400 MHz, CDCl₃) δ [ppm]=10.23 (s, 1H), 8.83 (s, 1H), 8.64 (s, 1H), 7.82 (d, 2H), 7.55 (t, 1H), 7.41 (t, 1H), 4.02 (s, 3H), 1.59 (s, 9H).

Intermediate 210

### methyl 3-[(tert-butoxycarbonyl)(methyl)amino]naphthalene-2-carboxylate

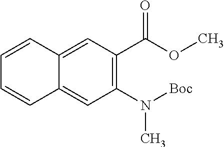 [SMILES: COC(=O)c1cc2ccccc2cc1N(C)C(=O)OC(C)(C)C]

To a solution of methyl 3-[(tert-butoxycarbonyl)amino]naphthalene-2-carboxylate (5.60 g, 90% purity, 16.7 mmol) in N,N-dimethylformamide (45 ml) was added sodium hydride (2.01 g, 60% purity, 50.2 mmol) at 0° C. under nitrogen atmosphere. After stirring at 25° C. for 1 hour, iodomethane (4.2 ml, 67 mmol) was added at 0° C. The reaction mixture was stirred at room temperature under nitrogen atmosphere for 2 hours. The mixture was quenched with saturated ammonium chloride, and extracted with ethyl acetate (combined with two other batches). The combined organic layers were washed with water and brine, dried over anhydrous sodium sulfate and concentrated under reduced pressure. The residue was purified by column chromatography on silica gel (1000 mesh, petroleum ether:ethyl acetate=20:1 to 5:1) to give methyl 3-[(tert-butoxycarbonyl)(methyl)amino]-2-naphthoate (8.30 g, 95% purity) as a yellow solid.

¹H NMR (400 MHz, CDCl₃) δ [ppm]=8.47 (s, 1H), 7.98-7.88 (m, 1H), 7.87-7.79 (m, 1H), 7.74-7.66 (m, 1H), 7.64-7.50 (m, 2H), 3.96 (s, 3H), 3.35 (s, 3H), 1.32 (s, 9H).

Intermediate 211

### [3-(methylamino)naphthalen-2-yl]MeOH

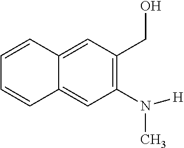

To a solution of methyl 3-[(tert-butoxycarbonyl)(methyl)amino]naphthalene-2-carboxylate (4.95 g, 95% purity, 14.91 mmol) in tetrahydrofuran (50 ml) was added lithium borohydride (974.5 mg, 44.73 mmol) at 0° C. After stirring at 50° C. for 16 hours, the mixture was quenched with saturated ammonium chloride and extracted with ethyl acetate. The organic phase was washed with brine, dried over anhydrous sodium sulfate, filtered and concentrated to give a residue. The residue was purified by column chromatography (1000 mesh, petroleum ether:ethyl acetate=5:1) to give [3-(methylamino)-2-naphthyl]methanol (3.34 g, 80% purity, 96% yield) as a white solid.

¹H NMR (400 MHz, CDCl₃) δ [ppm]=7.68 (d, 2H), 7.52 (s, 1H), 7.38 (t, 1H), 7.22 (t, 1H), 6.86 (s, 1H), 4.80 (s, 2H), 2.97 (s, 3H).

Intermediate 212

### 3-(methylamino)naphthalene-2-carbaldehyde

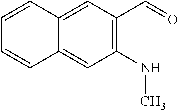

To a solution of [3-(methylamino)naphthalen-2-yl]methanol (3.92 g, 80% purity, 16.8 mmol) in acetonitrile (50 ml) was added manganese(IV) oxide (7.29 g, 83.8 mmol) at room temperature. After stirring at 80° C. for 16 hours, the suspension was filtered. The filtrate was concentrated under reduced pressure and purified by column chromatography (1000 mesh, petroleum ether) to give 3-(methylamino)-2-naphthaldehyde (1.94, 62% yield) as a yellow solid.

¹H NMR (400 MHz, CDCl₃) δ [ppm]=10.06 (s, 1H), 8.04 (s, 1H), 7.74 (d, 1H), 7.61 (d, 2H), 7.46 (t, 1H), 7.19 (t, 1H), 6.80 (s, 1H), 2.99 (s, 3H).

Intermediate 213

### tert-butyl (3-formylnaphthalen-2-yl)methylcarbamate

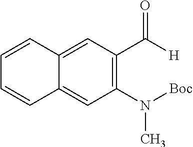 [SMILES: CN(C(=O)OC(C)(C)C)c1cc2ccccc2cc1C=O]

A solution of 3-(methylamino)naphthalene-2-carbaldehyde (2.01 g, 10.9 mol) in di-tert-butyl dicarbonate (5.0 ml, 22 mmol) was stirred at 100° C. for 16 hours. The mixture was purified by column chromatography (1000 mesh, petroleum ether:ethyl acetate=30:1 to 5:1) to give tert-butyl (3-formyl-2-naphthyl)methylcarbamate (1.58 g, 51% yield) as a white solid.

¹H NMR (400 MHz, CDCl₃) δ [ppm]=10.20 (s, 1H), 8.43 (s, 1H), 8.01 (d, 1H), 7.87 (d, 1H), 7.70 (s, 1H), 7.67-7.52 (m, 2H), 3.41 (s, 3H), 1.34 (s, 9H).

Intermediate 214

### methyl (2Z)-2-{[(benzyloxy)carbonyl]amino}-3-{3-[(tert-butoxycarbonyl)(methyl)amino]naphthalen-2-yl}prop-2-enoate

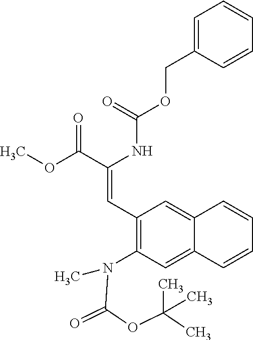 [SMILES: COC(=O)/C(=C/c1cc2ccccc2cc1N(C)C(=O)OC(C)(C)C)NC(=O)OCc1ccccc1]

1,8-Diazabicyclo(5.4.0)undec-7-ene (0.990 ml, 6.6 mmol) was added dropwise to a solution of methyl {[(benzyloxy)carbonyl]amino}(dimethoxyphosphoryl)acetate (2.20 g, 6.64 mmol) in dichloromethane (20 ml). After stirring at room temperature for 10 minutes, a solution of tert-butyl (3-formylnaphthalen-2-yl)methylcarbamate (1.58 g, 5.54 mmol) in dichloromethane (10 ml) was added. After stirring at room temperature for 2 hours, the reaction mixture was concentrated and diluted with ethyl acetate. The solution was washed with hydrochloric acid (1M) and brine. The organic phase was dried over anhydrous sodium sulfate and filtered. The filtrate was concentrated and purified by column chromatography (1000 mesh, petroleum ether:ethyl acetate=10:1 then 2:1) to give methyl (2Z)-2-{[(benzyloxy)carbonyl]amino}-3-(quinolin-3-yl)acrylate (1.78 g, 66% yield) as a yellow solid.

LC-MS (Method D): Rₜ=1.095 min; MS (ESIpos): m/z=513.1 [M+Na]⁺

¹H NMR (400 MHz, CDCl₃) δ [ppm]=7.93-7.87 (m, 1H), 7.70 (d, 1H), 7.61-7.57 (m, 2H), 7.40-7.39 (m, 2H), 7.22-7.18 (m, 6H), 6.38-6.37 (m, 1H), 5.01 (d, 2H), 3.75 (s, 3H), 3.15 (s, 3H), 1.40-1.26 (m, 9H).

Intermediate 215

### methyl (2R)-2-{[(benzyloxy)carbonyl]amino}-3-{3-[(tert-butoxycarbonyl)(methyl)amino]naphthalen-2-yl}propanoate

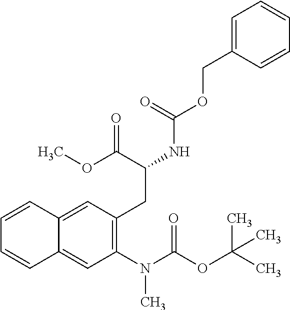 [SMILES: COC(=O)[C@@H](Cc1cc2ccccc2cc1N(C)C(=O)OC(C)(C)C)NC(=O)OCc1ccccc1]

To a solution of methyl methyl (2E)-2-{[(benzyloxy)carbonyl]amino}-3-{3-[(tert-butoxycarbonyl) (methyl)amino]naphthalen-2-yl}prop-2-enoate (1.48 g, 3.02 mmol) in methanol (50 ml) was added (R)—[Rh(COD)(MaxPhos)]BF₄ (179 mg, 0.302 mmol). After stirring at room temperature for 24 hours under hydrogen atmosphere (50 psi), the reaction mixture was evaporated under reduced pressure to give a residue. The residue was purified by column chromatography (1000 mesh, petroleum ether:ethyl acetate=2:1) to give methyl (2S)-2-{[(benzyloxy)carbonyl]amino}-3-{3-[(tert-butoxycarbonyl)(methyl)amino]-2-naphthyl}propanoate (1.48 g, 100% yield) as yellow oil.

Intermediate 216

### (2R)-2-{[(benzyloxy)carbonyl]amino}-3-{3-[(tert-butoxycarbonyl)(methyl)amino]naphthalen-2-yl}propanoic acid

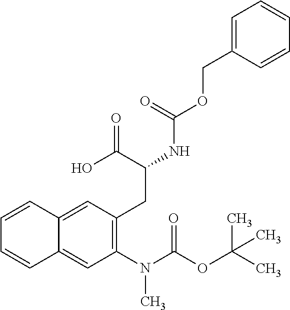 [SMILES: CN(C(=O)OC(C)(C)C)c1cc2ccccc2cc1C[C@@H](NC(=O)OCc1ccccc1)C(=O)O]

To a solution of methyl (2R)-2-{[(benzyloxy)carbonyl]amino}-3-{3-[(tert-butoxycarbonyl) (methyl)amino]naphthalen-2-yl}propanoate (1.45 g, 2.94 mmol) in tetrahydrofuran (15 ml) was added lithium hydroxide aqueous solution (1.8 ml, 2.0 M, 3.5 mmol). After stirring at room temperature for 1 hour, the pH of the solution was adjusted to 5 with hydrochloric acid (1 M). The reaction solution was concentrated under reduced pressure and dissolved in ethyl acetate. The solution was washed with water and brine. The organic phase was dried over anhydrous sodium sulfate, filtered and concentrated under reduced pressure. The residue (combined with another batch) was purified by preparative HPLC (Instrument: ACSWH-PREP-HPLC-D; Column: Phenomenex Synergi Max-RP 250*50 mm*10 μm; eluent A: 0.01% ammonium bicarbonate in water, eluent B: acetonitrile; gradient: 5-22 min 30-70% B; flow 100 ml/min; temperature: room temperature; Detector: UV 220/254 nm) to give (2S)-2-{[(benzyloxy)carbonyl]amino}-3-{3-[(tert-butoxycarbonyl)(methyl)amino]-2-naphthyl} propanoic acid (1.16 g, 99% purity, 82% yield) as a yellow solid.

LC-MS (Method C): Rₜ=0.965 min; MS (ESIpos): m/z=501.2 [M+Na]⁺.

¹H NMR (400 MHz, DMSO-d₆) δ [ppm]=7.86-7.72 (m, 4H), 7.49-7.47 (m, 2H), 7.31-7.06 (m, 6H), 4.97-4.88 (m, 2H), 4.33-4.13 (m, 1H), 3.35-3.31 (m, 1H), 3.20-3.15 (m, 3H), 2.95-2.84 (m, 1H), 1.49-1.26 (m, 9H).

Intermediate 217

### tri-tert-butyl (5R,12S,16S)-5-({3-[(tert-butoxycarbonyl)(methyl)amino]naphthalen-2-yl}methyl)-3,6,14-trioxo-1-phenyl-2-oxa-4,7,13,15-tetraazaoctadecane-12,16,18-tricarboxylate

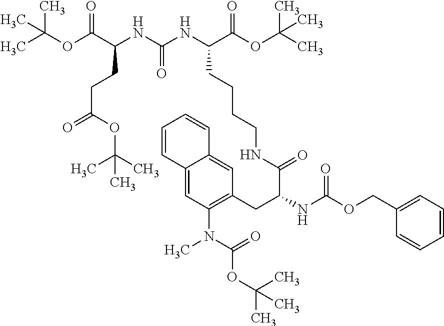 [SMILES: CN(C(=O)OC(C)(C)C)c1cc2ccccc2cc1C[C@@H](NC(=O)OCc1ccccc1)C(=O)NCCCC[C@H](NC(=O)N[C@@H](CCC(=O)OC(C)(C)C)C(=O)OC(C)(C)C)C(=O)OC(C)(C)C]

di-tert-butyl N-{[(2S)-6-amino-1-tert-butoxy-1-oxohexan-2-yl]carbamoyl}-L-glutamate (713 mg, 1.46 mmol) and (2R)-2-{[(benzyloxy)carbonyl]amino}-3-{3-[(tert-butoxycarbonyl)(methyl)amino]naphthalen-2-yl}propanoic acid (700 mg, 1.46 mmol) were solubilised in DMF (11 ml), 4-methylmorpholine (400 μl, 3.7 mmol, CAS-RN: 109-02-4) and HATU (779 mg, 2.05 mmol) were added and the mixture was stirred under argon overnight at rt. The mixture was evaporated and purified by preparative HPLC (C18, acetonitrile/water with 0.1% formic acid) to give 320 mg (95% purity, 22% yield) of the target compound.

LC-MS (Method 1): Rₜ=1.66 min; MS (ESIpos): m/z=949 [M+H]⁺

Intermediate 218

### di-tert-butyl (2S)-2-({[(2S)-6-{[(2R)-2-amino-3-{3-[(tert-butoxycarbonyl)(methyl)amino]naphthalen-2-yl}propanoyl]amino}-1-tert-butoxy-1-oxohexan-2-yl]carbamoyl}amino)pentanedioate

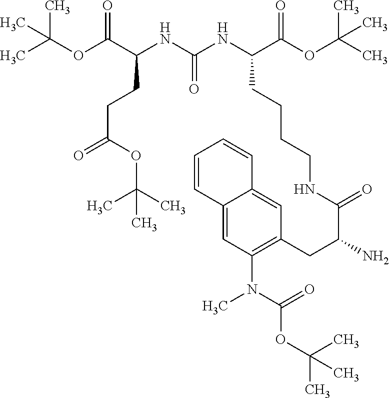 [SMILES: CN(C(=O)OC(C)(C)C)c1cc2ccccc2cc1C[C@@H](N)C(=O)NCCCC[C@H](NC(=O)N[C@@H](CCC(=O)OC(C)(C)C)C(=O)OC(C)(C)C)C(=O)OC(C)(C)C]

tri-tert-butyl (5R,12S,16S)-5-({3-[(tert-butoxycarbonyl)(methyl)amino]naphthalen-2-yl}methyl)-3,6,14-trioxo-1-phenyl-2-oxa-4,7,13,15-tetraazaoctadecane-12,16,18-tricarboxylate (456 mg, 481 μmol) was solubilised in MeOH (3.9 ml), Palladium on carbon (51.2 mg, 10% purity, 48.1 μmol) was added and the mixture was purged with hydrogen. The mixture was stirred for 3 h at rt under hydrogen atmosphere. The mixture was filtered over Celite, washed with MeOH and evaporated. The residue was purified by preparative HPLC (C18, acetonitrile/water with 0.1% formic acid) to give 227 mg (95% purity, 55% yield) of the target compound.

LC-MS (Method 1): Rₜ=1.27 min; MS (ESIpos): m/z=815 [M+H]⁺

¹H-NMR (400 MHz, DMSO-d6) δ[ppm]: 1.235 (1.14), 1.257 (1.03), 1.383 (16.00), 1.387 (11.18), 1.488 (0.48), 2.522 (1.09), 3.189 (0.56), 7.817 (0.56), 7.843 (0.45).

Intermediate 219

### tri-tert-butyl (3R,10S,14S)-3-({3-[(tert-butoxycarbonyl)(methyl)amino]naphthalen-2-yl}methyl)-1-{(1r,4S)-4-[({[(9H-fluoren-9-yl)methoxy]carbonyl}amino)methyl]cyclohexyl}-1,4,12-trioxo-2,5,11,13-tetraazahexadecane-10,14,16-tricarboxylate

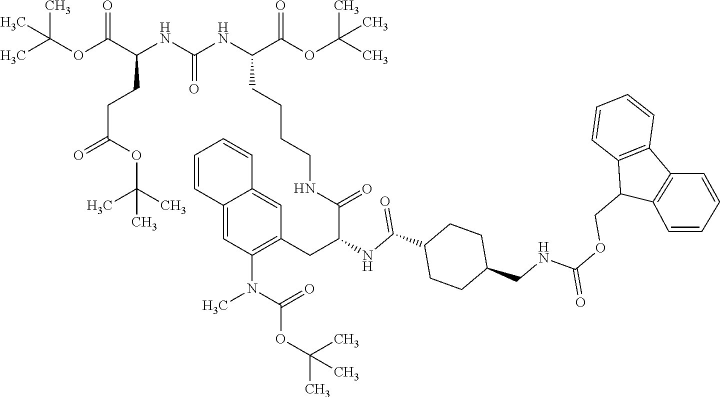

(1 r,4r)-4-[({[(9H-fluoren-9-yl)methoxy]carbonyl}amino)methyl]cyclohexane-1-carboxylic acid (111 mg, 291 μmol) was solubilised in DMF (4.1 ml), 4-methylmorpholine (87 μl, 790 μmol, CAS-RN: 109-02-4) and HATU (111 mg, 291 μmol) were added, stirred for 20 min at rt, di-tert-butyl (2S)-2-({[(2S)-6-{[(2R)-2-amino-3-{3-[(tert-butoxycarbonyl)(methyl)amino]naphthalen-2-yl}propanoyl]amino}-1-tert-butoxy-1-oxohexan-2-yl]carbamoyl}amino)pentanedioate (227 mg, 95% purity, 265 μmol) was added and the mixture was stirred under argon overnight at rt. The mixture was filtered and purified by preparative HPLC (C18, acetonitrile/water with 0.1% formic acid) to give 180 mg (98% purity, 57% yield) of the target compound.

LC-MS (Method 1): Rₜ=1.71 min; MS (ESIpos): m/z=1176 [M+H]⁺

Intermediate 220

### tri-tert-butyl (3R,10S,14S)-1-[(1r,4S)-4-(aminomethyl)cyclohexyl]-3-({3-[(tert-butoxycarbonyl)(methyl)amino]naphthalen-2-yl}methyl)-1,4,12-trioxo-2,5,11,13-tetraazahexadecane-10,14,16-tricarboxylate

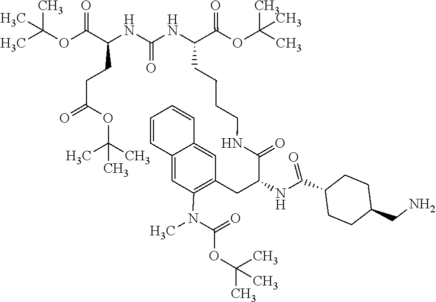 [SMILES: CN(C(=O)OC(C)(C)C)c1cc2ccccc2cc1C[C@@H](NC(=O)[C@H]1CC[C@H](CN)CC1)C(=O)NCCCC[C@H](NC(=O)N[C@@H](CCC(=O)OC(C)(C)C)C(=O)OC(C)(C)C)C(=O)OC(C)(C)C]

tri-tert-butyl (3R,10S,14S)-3-({3-[(tert-butoxycarbonyl)(methyl)amino]naphthalen-2-yl}methyl)-1-{(1r,4S)-4-[({[(9H-fluoren-9-yl)methoxy]carbonyl}amino)methyl]cyclohexyl}-1,4,12-trioxo-2,5,11,13-tetraazahexadecane-10,14,16-tricarboxylate (180 mg, 153 μmol) was solubilised in DMF (2.4 ml), piperidine (300 μl, 3.1 mmol) was added and the mixture was stirred under argon overnight at rt. The mixture was evaporated and purified by preparative HPLC (C18, acetonitrile/water with 0.1% formic acid) to give 151 mg (92% purity, 95% yield) of the target compound.

LC-MS (Method 1): Rₜ=1.29 min; MS (ESIpos): m/z=954 [M+H]⁺

¹H-NMR (400 MHz, DMSO-d6) δ[ppm]: 1.242 (1.48), 1.383 (16.00), 1.390 (10.35), 1.450 (0.42), 1.489 (0.59), 1.715 (1.27), 1.870 (1.17), 2.085 (3.32), 2.518 (1.32), 2.523 (0.87), 3.194 (0.60).

### Example 39-A

### (3R,10S,14S)-1-[(1r,4S)-4-(aminomethyl)cyclohexyl]-3-{[3-(methylamino)naphthalen-2-yl]methyl}-1,4,12-trioxo-2,5,11,13-tetraazahexadecane-10,14,16-tricarboxylic acid

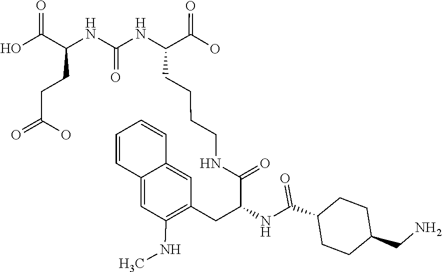

tri-tert-butyl (3R,10S,14S)-1-[(1r,4S)-4-(aminomethyl)cyclohexyl]-3-({3-[(tert-butoxycarbonyl)(methyl)amino]naphthalen-2-yl}methyl)-1,4,12-trioxo-2,5,11,13-tetraazahexadecane-10,14,16-tricarboxylate (11.3 mg, 11.9 μmol) was solubilised in DCM (380 μl), TFA (180 μl, 2.4 mmol) was added and the mixture was stirred under argon overnight at rt.

The mixture was evaporated and purified by preparative HPLC to give 2.00 mg (90% purity, 22% yield) of the target compound.

LC-MS (Method 1): Rₜ=0.74 min; MS (ESIpos): m/z=686 [M+H]⁺

¹H-NMR (400 MHz, DMSO-d6) δ[ppm]: 0.852 (1.30), 0.886 (1.19), 0.913 (0.54), 1.148 (0.70), 1.175 (0.76), 1.233 (2.16), 1.263 (2.27), 1.291 (1.84), 1.328 (0.86), 1.352 (0.81), 1.383 (1.03), 1.430 (1.35), 1.444 (1.41), 1.544 (0.92), 1.594 (1.19), 1.607 (1.35), 1.627 (1.51), 1.643 (1.57), 1.679 (1.46), 1.715 (0.81), 1.763 (0.92), 1.786 (1.19), 2.052 (0.54), 2.081 (0.92), 2.111 (0.49), 2.145 (0.54), 2.163 (0.86), 2.181 (0.92), 2.194 (0.59), 2.227 (0.54), 2.249 (0.92), 2.269 (0.65), 2.284 (0.59), 2.327 (3.14), 2.332 (2.27), 2.336 (1.08), 2.518 (16.00), 2.523 (9.78), 2.539 (2.32), 2.596 (1.78), 2.669 (3.19), 2.673 (2.32), 2.823 (9.95), 2.848 (1.19), 2.872 (0.97), 2.978 (1.73), 2.989 (1.78), 3.012 (1.24), 3.026 (1.08), 3.099 (1.35), 3.117 (1.30), 3.933 (1.78), 3.944 (1.78), 4.444 (0.54), 4.466 (0.97), 4.478 (1.03), 4.500 (0.49), 6.139 (0.59), 6.424 (0.49), 6.668 (4.92), 7.059 (1.08), 7.079 (2.11), 7.097 (1.30), 7.237 (1.30), 7.255 (2.11), 7.272 (1.14), 7.432 (4.22), 7.513 (2.11), 7.533 (1.95), 7.570 (2.38), 7.591 (2.05), 7.967 (0.54), 8.355 (0.43).

Intermediate 221

### tri-tert-butyl (3R,10S,14S)-3-({3-[(tert-butoxycarbonyl)(methyl)amino]naphthalen-2-yl}methyl)-1,4,12-trioxo-1-[(1r,4S)-4-({2-[4,7,10-tris(2-tert-butoxy-2-oxoethyl)-1,4,7,10-tetraazacyclododecan-1-yl]acetamido}methyl)cyclohexyl]-2,5,11,13-tetraazahexadecane-10,14,16-tricarboxylate

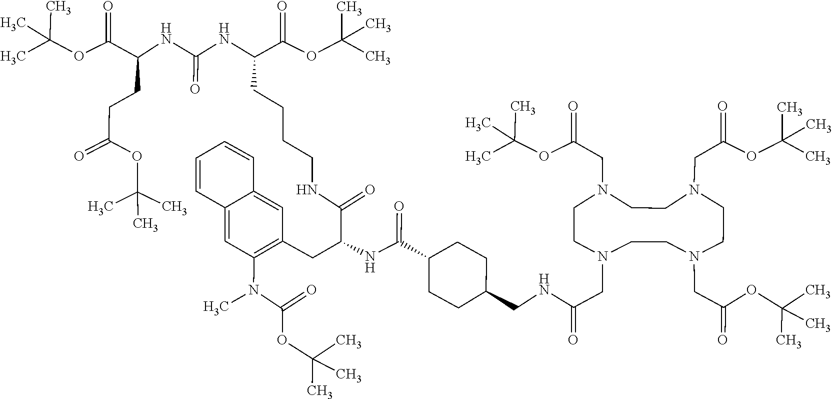 [SMILES: CN(C(=O)OC(C)(C)C)c1cc2ccccc2cc1C[C@@H](NC(=O)[C@H]1CC[C@H](CNC(=O)CN2CCN(CC(=O)OC(C)(C)C)CCN(CC(=O)OC(C)(C)C)CCN(CC(=O)OC(C)(C)C)CC2)CC1)C(=O)NCCCC[C@H](NC(=O)N[C@@H](CCC(=O)OC(C)(C)C)C(=O)OC(C)(C)C)C(=O)OC(C)(C)C]

[4,7,10-tris(2-tert-butoxy-2-oxoethyl)-1,4,7,10-tetraazacyclododecan-1-yl]acetic acid (66.3 mg, 116 μmol; CAS-RN:[137076-54-1]), [(1H-benzotriazol-1-yl)oxy](dimethylamino)-N,N-dimethylmethaniminium hexafluoridophosphate(1−) (43.4 mg, 114 μmol; CAS-RN:94790-37-1) and N,N-diisopropylethylamine (15 μl, 87 μmol) were stirred in DMF (3.0 ml) for 10 min at rt. tri-tert-butyl (3R,10S,14S)-1-[(1r,4S)-4-(aminomethyl)cyclohexyl]-3-({3-[(tert-butoxycarbonyl)(methyl)amino]naphthalen-2-yl}methyl)-1,4,12-trioxo-2,5,11,13-tetraazahexadecane-10,14,16-tricarboxylate (30.0 mg, 92% purity, 29.0 μmol) was added and the mixture was stirred for 3 h at rt. The mixture was evaporated and purified by preparative HPLC (C18, acetonitrile/water with 0.1% formic acid) to give 42.0 mg (96% yield) of the target compound.

LC-MS (Method 1): Rₜ=1.36 min; MS (ESIpos): m/z=1509 [M+H]⁺

### Example 39-B

### (3R,10S,14S)-3-{[3-(methylamino)naphthalen-2-yl]methyl}-1,4,12-trioxo-1-[(1r,4S)-4-({2-[4,7,10-tris(carboxymethyl)-1,4,7,10-tetraazacyclododecan-1-yl]acetamido}methyl)cyclohexyl]-2,5,11,13-tetraazahexadecane-10,14,16-tricarboxylic acid

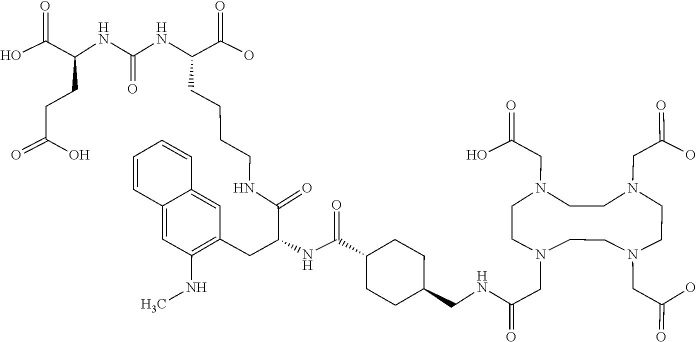

tri-tert-butyl (3R,10S,14S)-3-({3-[(tert-butoxycarbonyl)(methyl)amino]naphthalen-2-yl}methyl)-1,4,12-trioxo-1-[(1r,4S)-4-({2-[4,7,10-tris(2-tert-butoxy-2-oxoethyl)-1,4,7,10-tetraazacyclododecan-1-yl]acetamido}methyl)cyclohexyl]-2,5,11,13-tetraazahexadecane-10,14,16-tricarboxylate (46.0 mg, 30.5 μmol) was solubilised in DCM (3.0 ml), TFA (700 μl, 9.15 μmol) was added and the mixture was stirred under argon for 2 h at rt. The mixture was evaporated and purified by preparative HPLC (C18, acetonitrile/water with 0.1% formic acid) to give 4.10 mg (90% purity, 11% yield) of the target compound.

LC-MS (Method 1): Rₜ=0.78 min; MS (ESIpos): m/z=1072 [M+H]⁺

¹H-NMR (400 MHz, DMSO-d6) δ[ppm]: 0.833 (0.52), 0.852 (0.46), 1.232 (1.64), 1.308 (0.59), 1.353 (0.92), 1.605 (0.59), 1.639 (0.59), 1.672 (0.59), 1.706 (0.59), 1.907 (1.70), 2.085 (0.39), 2.210 (0.52), 2.231 (0.72), 2.238 (0.66), 2.260 (0.46), 2.337 (1.38), 2.518 (16.00), 2.523 (10.69), 2.674 (3.02), 2.679 (1.44), 2.828 (5.64), 2.983 (2.43), 3.090 (0.98), 3.430 (3.08), 3.976 (0.46), 3.989 (0.46), 4.066 (0.46), 4.078 (0.39), 4.877 (0.39), 4.903 (0.46), 6.294 (0.66), 6.685 (2.03), 7.067 (0.46), 7.084 (0.85), 7.104 (0.52), 7.154 (0.39), 7.206 (0.46), 7.223 (0.59), 7.236 (0.66), 7.256 (0.85), 7.276 (0.46), 7.411 (1.51), 7.516 (0.85), 7.535 (0.72), 7.574 (0.92), 7.594 (0.79), 7.941 (0.59), 8.084 (0.52), 8.137 (0.79), 8.452 (0.66), 8.467 (0.59), 9.203 (0.59).

#40 Linker

Intermediate 222

### tri-tert-butyl (5S,12S,16S)-1-{(1r,4S)-4-[(tert-butoxycarbonyl)amino]cyclohexyl}-5-[(naphthalen-2-yl)methyl]-3,6,14-trioxo-2,4,7,13,15-pentaazaoctadecane-12,16,18-tricarboxylate

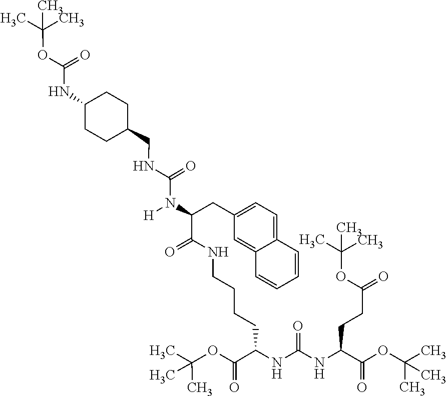 [SMILES: CC(C)(C)OC(=O)CC[C@H](NC(=O)N[C@@H](CCCCNC(=O)[C@H](Cc1ccc2ccccc2c1)NC(=O)NC[C@H]1CC[C@H](NC(=O)OC(C)(C)C)CC1)C(=O)OC(C)(C)C)C(=O)OC(C)(C)C]

tert-butyl [(1r,4r)-4-(aminomethyl)cyclohexyl]carbamate (5.87 mg, 25.7 μmol) was solubilised in DCM (450 μl, 7.0 mmol), N,N-diisopropylethylamine (20 μl, 120 μmol) and tri-tert-butyl (3S,10S,14S)-3-[(naphthalen-2-yl)methyl]-1-(4-nitrophenoxy)-1,4,12-trioxo-2,5,11,13-tetraazahexadecane-10,14,16-tricarboxylate (19.9 mg, 23.4 μmol) were added. The mixture was stirred under argon over the weekend at rt. The mixture was filtered and purified by preparative HPLC (C18, acetonitrile/water with 0.1% formic acid) to give 17.0 mg (90% purity, 70% yield) of the target compound.

LC-MS (Method 1): Rₜ=1.55 min; MS (ESIpos): m/z=940 [M+H]⁺

¹H-NMR (400 MHz, DMSO-d6) δ[ppm]: 1.232 (0.54), 1.352 (2.17), 1.367 (7.47), 1.380 (16.00), 1.382 (14.02), 2.331 (0.51), 2.518 (2.65), 2.523 (1.76), 2.673 (0.51), 7.621 (0.42), 7.780 (0.54).

### Example 40-A

### (5S,12S,16S)-1-[(1r,4S)-4-aminocyclohexyl]-5-[(naphthalen-2-yl)methyl]-3,6,14-trioxo-2,4,7,13,15-pentaazaoctadecane-12,16,18-tricarboxylic acid

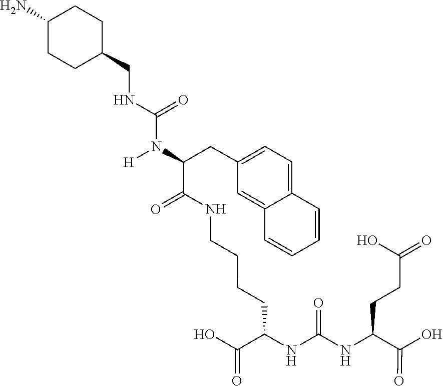 [SMILES: N[C@H]1CC[C@H](CNC(=O)N[C@@H](Cc2ccc3ccccc3c2)C(=O)NCCCC[C@H](NC(=O)N[C@@H](CCC(=O)O)C(=O)O)C(=O)O)CC1]

tri-tert-butyl (5S,12S,16S)-1-{(1r,4S)-4-[(tert-butoxycarbonyl)amino]cyclohexyl}-5-[(naphthalen-2-yl)methyl]-3,6,14-trioxo-2,4,7,13,15-pentaazaoctadecane-12,16,18-tricarboxylate (16.0 mg, 17.0 μmol) was solubilised in DCM (540 μl), TFA (19.4 mg, 170 μmol) was added and the mixture was stirred under argon overnight at rt. The mixture was evaporated and purified by preparative HPLC (C18, acetonitrile/water with 0.1% formic acid) to give 10.0 mg (95% purity, 83% yield) of the target compound.

LC-MS (Method 1): Rₜ=0.71 min; MS (ESIpos): m/z=671 [M+H]⁺

¹H-NMR (400 MHz, DMSO-d6) δ[ppm]: 0.813 (0.39), 0.844 (1.27), 0.875 (1.40), 0.909 (0.61), 1.170 (1.97), 1.202 (2.45), 1.224 (3.28), 1.232 (3.50), 1.249 (2.05), 1.269 (1.05), 1.286 (1.62), 1.303 (1.62), 1.432 (0.66), 1.450 (0.83), 1.468 (0.70), 1.571 (0.61), 1.584 (0.87), 1.605 (1.44), 1.634 (1.53), 1.688 (0.61), 1.705 (0.70), 1.722 (0.92), 1.734 (0.79), 1.738 (0.70), 1.845 (1.62), 1.864 (1.75), 1.877 (1.84), 1.898 (0.96), 1.907 (0.83), 2.216 (1.22), 2.225 (1.31), 2.237 (1.75), 2.242 (1.79), 2.263 (1.05), 2.298 (0.66), 2.331 (1.79), 2.336 (0.83), 2.518 (11.41), 2.522 (7.13), 2.539 (16.00), 2.546 (4.81), 2.673 (1.84), 2.678 (0.87), 2.727 (1.75), 2.762 (1.53), 2.777 (2.62), 2.793 (1.53), 2.866 (1.44), 2.888 (3.19), 2.900 (1.44), 2.921 (1.09), 2.979 (1.27), 2.993 (1.70), 3.007 (1.44), 3.021 (1.66), 3.035 (1.40), 3.055 (1.01), 3.067 (0.96), 3.090 (0.48), 3.725 (0.39), 3.738 (0.44), 3.985 (0.57), 3.997 (0.74), 4.005 (1.14), 4.018 (1.18), 4.038 (0.70), 4.063 (1.09), 4.078 (1.09), 4.098 (0.52), 4.380 (0.57), 4.400 (1.14), 4.415 (1.09), 4.435 (0.52), 6.144 (0.61), 6.200 (0.87), 6.283 (2.45), 6.303 (2.36), 7.326 (1.92), 7.330 (1.88), 7.348 (1.97), 7.351 (2.01), 7.423 (0.66), 7.427 (0.83), 7.440 (1.97), 7.444 (1.88), 7.452 (2.05), 7.457 (3.76), 7.463 (2.19), 7.471 (1.84), 7.476 (1.92), 7.488 (0.83), 7.492 (0.61), 7.634 (3.72), 7.783 (4.46), 7.796 (2.19), 7.803 (3.58), 7.840 (1.88), 7.845 (1.92), 7.863 (1.79), 7.935 (0.92), 7.949 (1.88), 7.962 (0.83).

#41 Linker

Intermediate 223

### 1-[(4-chlorophenyl)sulfanyl]propan-2-one

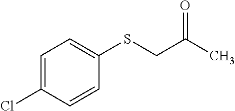

To a solution of 4-chlorobenzene-1-thiol (10.0 g, 69.1 mmol) and 1-chloropropan-2-one (6.72 g, 72.6 mmol) in N,N-dimethylformamide (100 ml) was added potassium carbonate (19.1 g, 138 mmol) at 0° C. The reaction mixture was stirred at room temperature for 16 hours. The mixture was poured into water and extracted with ethyl acetate. The organic phase was washed with brine, dried over anhydrous sodium sulfate, filtered and concentrated to give a residue. The residue was purified by column chromatography (1000 mesh, petroleum ether:ethyl acetate=30:1 to 5:1) to give 1-[(4-chlorophenyl)sulfanyl]acetone (5.00 g, 99% purity) and 1-[(4-chlorophenyl)sulfanyl]acetone (7.00 g, 83% purity) as yellow oil.

LC-MS (Method C): Rₜ=0.875 min; MS (ESIpos): m/z=200.9 [M+H]⁺.

¹H NMR (400 MHz, CDCl₃) δ [ppm]=7.27 (s, 4H), 3.65 (s, 2H), 2.28 (s, 3H).

Intermediate 224

### 5-chloro-3-methyl-1-benzothiophene

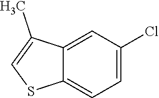

To a solution of 1-[(4-chlorophenyl)sulfanyl]propan-2-one (7.00 g, 83% purity, 29.0 mmol) in toluene (100 ml) was added polyphosphoric acid (29.3 g, 86.9 mmol) at room temperature. The reaction mixture was refluxed for 16 hours. The mixture (combined with another batch) was concentrated, dissolved in water and extracted with ethyl acetate. The organic phase was washed with sodium hydroxide (1M), brine, dried over anhydrous sodium sulfate, filtered and concentrated to give a residue. The residue was purified by column chromatography (1000 mesh, petroleum ether) to give 5-chloro-3-methyl-1-benzothiophene (7.00 g) as yellow oil.

¹H NMR (400 MHz, CDCl₃) δ [ppm]=7.76 (d, 1H), 7.70 (d, 1H), 7.32 (dd, 1H), 7.14 (s, 1H), 2.43 (s, 3H).

Intermediate 225

### 3-(bromomethyl)-5-chloro-1-benzothiophene

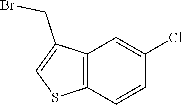 [SMILES: Clc1ccc2scc(CBr)c2c1]

To a solution of 5-chloro-3-methyl-1-benzothiophene (6.00 g, 32.8 mmol) in carbon tetrachloride (60 ml, 620 mmol) were added N-bromosuccinimide (5.85 g, 32.8 mmol) and dibenzoyl peroxide (159 mg, 0.657 mmol) at room temperature. After stirring at 80° C. for 12 hours, the reaction mixture was cooled to room temperature. and diluted with water. The mixture was extracted with dichloromethane. The organic layer was evaporated under reduced pressure and triturated with methyl tert-butyl ether to give 3-(bromomethyl)-5-chloro-1-benzothiophene (5.60 g, 65% yield) as a light yellow solid.

¹H NMR (400 MHz, DMSO-d₆) δ [ppm]=8.09-8.04 (m, 2H), 8.00 (d, 1H), 7.45 (dd, 1H), 5.04 (s, 2H).

Intermediate 226

### (5-chloro-1-benzothiophen-3-yl)mEtOAc

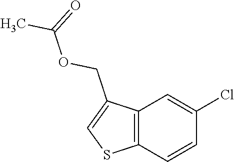

To a solution of 3-(bromomethyl)-5-chloro-1-benzothiophene (5.60 g, 21.4 mmol) in N,N-dimethylformamide (80 ml) were added sodium acetate (5.27 g, 64.2 mmol) and potassium iodide (711 mg, 4.28 mmol) at room temperature. After stirring at 50° C. for 12 hours, the reaction mixture was cooled to room temperature and diluted with water. The mixture was extracted with ethyl acetate. The organic layer was evaporated under reduced pressure to give (5-chloro-1-benzothiophen-3-yl)methyl acetate (5.10 g, 99% yield) as yellow oil.

¹H NMR (400 MHz, DMSO-d₆) δ=8.06 (d, 1H), 7.97-7.93 (m, 2H), 7.44 (dd, 1H), 5.32 (s, 2H), 2.06 (s, 3H).

Intermediate 227

### (5-chloro-1-benzothiophen-3-yl)MeOH

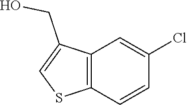 [SMILES: OCc1csc2ccc(Cl)cc12]

To a solution of (5-chloro-1-benzothiophen-3-yl)methyl acetate (5.10 g, 21.2 mmol) in methanol (80 ml) was added potassium carbonate (5.86 g, 42.4 mmol) at room temperature. After stirring at room temperature for 12 hours, the reaction mixture was diluted with water and extracted with ethyl acetate. The organic layer was evaporated under reduced pressure and purified by chromatography (100 mesh, petroleum ether:ethyl acetate=1:0, then 100:1, then 20:1) to give (5-chloro-1-benzothiophen-3-yl)methanol (4.00 g, 95% yield) as a yellow solid.

¹H NMR (400 MHz, DMSO-d₆) δ [ppm]=8.01 (d, 1H), 7.92 (d, 1H), 7.67 (s, 1H), 7.39 (dd, 1H), 5.29 (t, 1H), 4.72 (dd, 2H).

Intermediate 228

### 5-chloro-1-benzothiophene-3-carbaldehyde

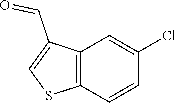 [SMILES: O=Cc1csc2ccc(Cl)cc12]

To a solution of (5-chloro-1-benzothiophen-3-yl)methanol (4.00 g, 20.1 mmol) in acetonitrile (50 ml) was added manganese(IV) oxide (8.75 g, 101 mmol) at room temperature. The reaction mixture was heated to 80° C. and stirred at 80° C. for 12 hours. The reaction mixture was cooled to room temperature and filtered through celite. The filtrate was evaporated under reduced pressure to give 5-chloro-1-benzothiophene-3-carbaldehyde (4.00 g) as a yellow solid.

¹H NMR (400 MHz, DMSO-d₆) δ [ppm]=10.10 (s, 1H), 9.06 (s, 1H), 8.50 (d, 1H), 8.16 (d, 1H), 7.54 (dd, 1H), 5.30 (t, 1H), 4.71 (dd, 2H).

Intermediate 229

### methyl (2Z)-2-{[(benzyloxy)carbonyl]amino}-3-(5-chloro-1-benzothiophen-3-yl)prop-2-enoate

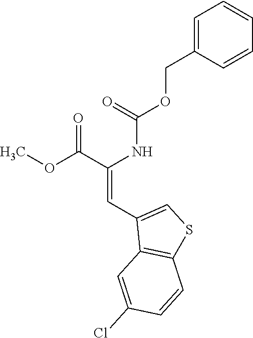 [SMILES: COC(=O)/C(=C/c1csc2ccc(Cl)cc12)NC(=O)OCc1ccccc1]

1,8-Diazabicyclo(5.4.0)undec-7-ene was added dropwise to a solution of methyl (2R)—{[(benzyloxy)carbonyl]amino}(dimethoxyphosphoryl)acetate (8.09 g, 24.4 mmol) in dichloromethane (50 mL). After stirring at room temperature for 10 minutes, a solution of 5-chloro-1-benzothiophene-3-carbaldehyde (4.00 g, 20.3 mmol) in dichloromethane (30 ml) was added. The reaction was stirred at room temperature for 2 hours. The reaction solvent was removed under reduced pressure to give a residue. The residue was diluted with ethyl acetate (40 mL). The solution was washed with 1M HCl and brine. The organic phase was dried over anhydrous sodium sulfate and filtered. The filtrate was concentrated and purified by column chromatography (1000 mesh, petroleum ether:ethyl acetate=1:0, then 100:1, then 70:1, then 40:1, then 10:1) to give methyl (2Z)-2-{[(benzyloxy)carbonyl]amino}-3-(5-chloro-1-benzothiophen-3-yl)acrylate as a white solid.

LC-MS (Method D): Rₜ=0.921 min; MS (ESIpos): m/z=402.0 [M+H]⁺.

¹H NMR (400 MHz, DMSO-d₆) δ [ppm]=9.32 (s, 1H), 8.26 (s, 1H), 8.10 (d, 1H), 8.04 (d, 1H), 7.54-7.45 (m, 2H), 7.43-7.22 (m, 5H), 5.11 (s, 2H), 3.76 (s, 3H).

Intermediate 230

### methyl N-[(benzyloxy)carbonyl]-3-(5-chloro-1-benzothiophen-3-yl)-D-alaninate

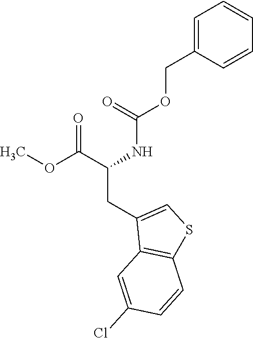 [SMILES: COC(=O)[C@@H](Cc1csc2ccc(Cl)cc12)NC(=O)OCc1ccccc1]

To a solution of methyl (2Z)-2-{[(benzyloxy)carbonyl]amino}-3-(5-chloro-1-benzothiophen-3-yl)prop-2-enoate (3.90 g, 9.70 mmol) in methanol (150 ml) were added (R)—[Rh(COD)(MaxPhos)]BF₄ (0.06 eq) (345 mg, 0.582 mmol). After stirring at room temperature for 16 hours under hydrogen atmosphere (50 psi), the reaction mixture (combined with another batch) was evaporated under reduced pressure to give a residue. The residue was dissolved in dichloromethane and filtered through silica gel (200-300 mesh). The filtrate was evaporated under reduced pressure to give methyl N-[(benzyloxy)carbonyl]-3-(5-chloro-1-benzothiophen-3-yl)-L-alaninate (4.00 g, 97% purity, 99% yield) as a yellow oil.

LC-MS (Method D): Rₜ=0.928 min; MS (ESIpos): m/z=404.0 [M+H]⁺.

Intermediate 231

### N-[(benzyloxy)carbonyl]-3-(5-chloro-1-benzothiophen-3-yl)-D-alanine

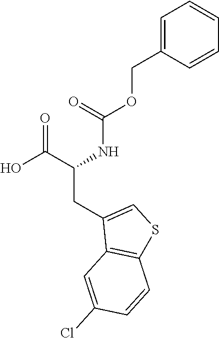 [SMILES: O=C(N[C@H](Cc1csc2ccc(Cl)cc12)C(=O)O)OCc1ccccc1]

To a solution of methyl N-[(benzyloxy)carbonyl]-3-(5-chloro-1-benzothiophen-3-yl)-D-alaninate (3.80 g, 97% purity, 9.13 mmol) in tetrahydrofuran (70 ml) was added lithium hydroxide (2M in water) (15 ml, 2.0 M, 30 mmol) at 0° C. After stirring at room temperature for 12 hours, the pH of the mixture was adjusted to 5-6 with formic acid. The mixture was concentrated and purified by preparative HPLC (Instrument: Shimadzu LC-20AP; Column: Phenomenex luna C18 250*100 mm*10 μm; eluent A: 0.225% formic acid in water, eluent B: acetonitrile; gradient: 0-20 min 40-75% B; flow 100 ml/min; temperature: room temperature; Detector: UV 220/254 nm) to give N-[(benzyloxy)carbonyl]-3-(5-chloro-1-benzothiophen-3-yl)-L-alanine (3.18 g, 99% purity, 88% yield) as a light yellow solid.

LC-MS (Method D): Rₜ=0.857 min; MS (ESIpos): m/z=411.9 [M+23]⁺.

¹H NMR (400 MHz, DMSO-d₆) δ [ppm]=8.01 (d, 1H), 7.91 (d, 1H), 7.72 (d, 1H), 7.57 (s, 1H), 7.40 (dd, 1H), 7.35-7.21 (m, 5H), 4.96 (s, 2H), 4.36-4.25 (m, 1H), 3.28-3.25 (m, 1H), 3.13 (dd, 1H).

Intermediate 232

### tri-tert-butyl (5R,12S,16S)-5-[(5-chloro-1-benzothiophen-3-yl)methyl]-3,6,14-trioxo-1-phenyl-2-oxa-4,7,13,15-tetraazaoctadecane-12,16,18-tricarboxylate

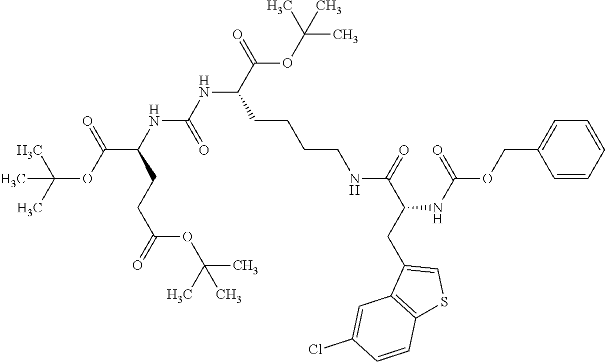 [SMILES: CC(C)(C)OC(=O)CC[C@H](NC(=O)N[C@@H](CCCCNC(=O)[C@@H](Cc1csc2ccc(Cl)cc12)NC(=O)OCc1ccccc1)C(=O)OC(C)(C)C)C(=O)OC(C)(C)C]

di-tert-butyl N-{[(2S)-6-amino-1-tert-butoxy-1-oxohexan-2-yl]carbamoyl}-L-glutamate (876 mg, 1.80 mmol) and N-[(benzyloxy)carbonyl]-3-(5-chloro-1-benzothiophen-3-yl)-D-alanine (700 mg, 1.80 mmol) were solubilised in DMF (14 ml), 4-methylmorpholine (490 μl, 4.5 mmol, CAS-RN: 109-02-4) and HATU (956 mg, 2.51 mmol) were added and the mixture was stirred under argon overnight at rt. The mixture was evaporated and purified by preparative HPLC (C18, acetonitrile/water with 0.1% formic acid) to give 559 mg (36% yield) of the target compound.

LC-MS (Method 1): Rₜ=1.61 min; MS (ESIpos): m/z=861 [M+H]⁺

Intermediate 233

### di-tert-butyl (2S)-2-({[(2S)-6-{[(2R)-2-amino-3-(5-chloro-1-benzothiophen-3-yl)propanoyl]amino}-1-tert-butoxy-1-oxohexan-2-yl]carbamoyl}amino)pentanedioate

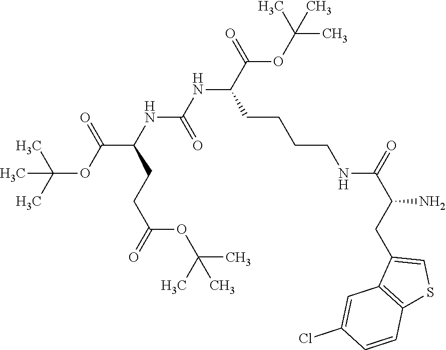 [SMILES: CC(C)(C)OC(=O)CC[C@H](NC(=O)N[C@@H](CCCCNC(=O)[C@H](N)Cc1csc2ccc(Cl)cc12)C(=O)OC(C)(C)C)C(=O)OC(C)(C)C]

tri-tert-butyl (5R,12S,16S)-5-[(5-chloro-1-benzothiophen-3-yl)methyl]-3,6,14-trioxo-1-phenyl-2-oxa-4,7,13,15-tetraazaoctadecane-12,16,18-tricarboxylate (559 mg, 98% purity, 637 μmol) was solubilised in MeOH (5.2 ml), Palladium on carbon (67.8 mg, 10% purity, 63.7 μmol) was added and the mixture was purged with hydrogen. The mixture was stirred for 3.5 h at rt under hydrogen atmosphere. The mixture was filtered over Celite, washed with MeOH and evaporated. The residue was purified by preparative HPLC (C18, acetonitrile/water with 0.1% formic acid) to give 24.8 mg (5% yield) of the target compound.

LC-MS (Method 1): Rt=1.24 min; MS (ESIpos): m/z=726 [M+H]⁺

¹H-NMR (400 MHz, DMSO-d6) δ[ppm]: 1.107 (2.19), 1.382 (15.28), 1.388 (16.00), 2.327 (0.53), 2.518 (2.18), 2.522 (1.36), 2.669 (0.55), 7.550 (0.87), 7.892 (0.60), 7.897 (0.59), 7.980 (0.67), 8.002 (0.63).

Intermediate 234

### tri-tert-butyl (3R,10S,14S)-3-[(5-chloro-1-benzothiophen-3-yl)methyl]-1-{(1r,4S)-4-[({[(9H-fluoren-9-yl)methoxy]carbonyl}amino)methyl]cyclohexyl}-1,4,12-trioxo-2,5,11,13-tetraazahexadecane-10,14,16-tricarboxylate

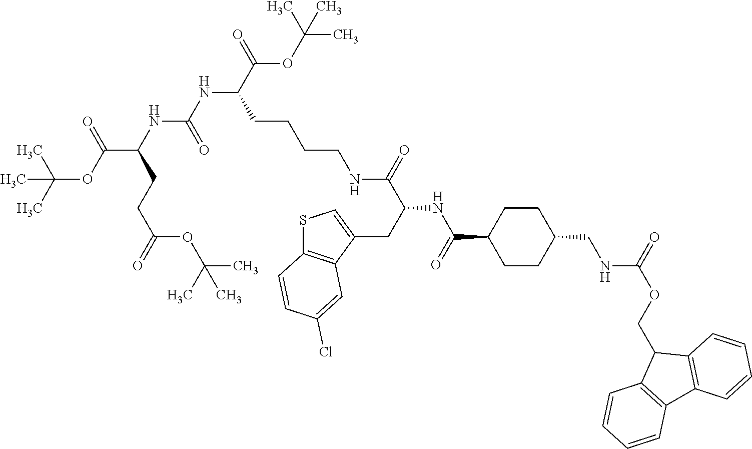 [SMILES: CC(C)(C)OC(=O)CC[C@H](NC(=O)N[C@@H](CCCCNC(=O)[C@@H](Cc1csc2ccc(Cl)cc12)NC(=O)[C@H]1CC[C@H](CNC(=O)OCC2c3ccccc3-c3ccccc32)CC1)C(=O)OC(C)(C)C)C(=O)OC(C)(C)C]

(1r,4r)-4-[({[(9H-fluoren-9-yl)methoxy]carbonyl}amino)methyl]cyclohexane-1-carboxylic acid (13.8 mg, 36.4 μmol) was solubilised in DMF (510 μl), 4-methylmorpholine (11 μl, 99 μmol, CAS-RN: 109-02-4) and HATU (13.8 mg, 36.4 μmol) were added, stirred for 20 min at rt, di-tert-butyl (2S)-2-({[(2S)-6-{[(2R)-2-amino-3-(5-chloro-1-benzothiophen-3-yl)propanoyl]amino}-1-tert-butoxy-1-oxohexan-2-yl]carbamoyl}amino)pentanedioate (24.0 mg, 33.1 μmol) was added and the mixture was stirred under argon overnight at rt. The mixture was filtered and evaporated to give 35.0 mg (15% purity, 15% yield) of the target compound.

LC-MS (Method 1): Rₜ=1.67 min; MS (ESIpos): m/z=1087 [M+H]⁺

Intermediate 235

tri-tert-butyl (3R,10S,14S)-1-[(1r,4S)-4-(aminomethyl)cyclohexyl]-3-[(5-chloro-1-benzothiophen-3-yl)methyl]-1,4,12-trioxo-2,5,11,13-tetraazahexadecane-10,14,16-tricarboxylate

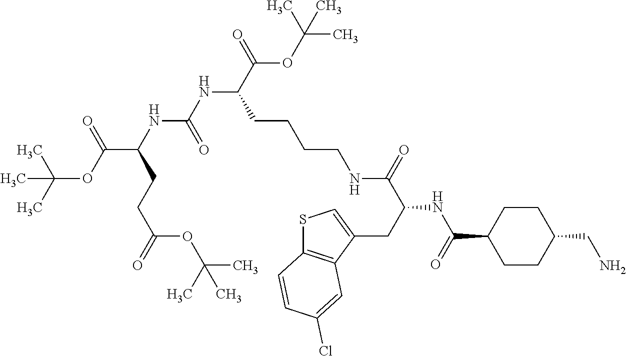 [SMILES: CC(C)(C)OC(=O)CC[C@H](NC(=O)N[C@@H](CCCCNC(=O)[C@@H](Cc1csc2ccc(Cl)cc12)NC(=O)[C@H]1CC[C@H](CN)CC1)C(=O)OC(C)(C)C)C(=O)OC(C)(C)C]

tri-tert-butyl (3R,10S,14S)-3-[(5-chloro-1-benzothiophen-3-yl)methyl]-1-{(1r,4S)-4-[({[(9H-fluoren-9-yl)methoxy]carbonyl}amino)methyl]cyclohexyl}-1,4,12-trioxo-2,5,11,13-tetraazahexadecane-10,14,16-tricarboxylate (35.0 mg, 32.2 μmol) was solubilised in DMF (500 μl), piperidine (64 μl, 640 μmol) was added and the mixture was stirred under argon for 2 h at rt. The mixture was evaporated and purified by preparative HPLC (C18, acetonitrile/water with 0.1% formic acid) to give 12.0 mg (95% purity, 41% yield) of the target compound.

LC-MS (Method 1): Rₜ=1.28 min; MS (ESIpos): m/z=866 [M+H]⁺

### Example 41-A

### N⁶—{N-[(1r,4S)-4-(aminomethyl)cyclohexane-1-carbonyl]-3-(5-chloro-1-benzothiophen-3-yl)-D-alanyl}-N²-{[(1S)-1,3-dicarboxypropyl]carbamoyl}-L-lysine

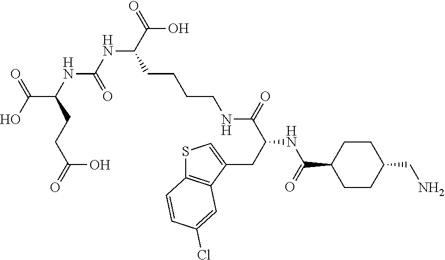 [SMILES: NC[C@H]1CC[C@H](C(=O)N[C@H](Cc2csc3ccc(Cl)cc23)C(=O)NCCCC[C@H](NC(=O)N[C@@H](CCC(=O)O)C(=O)O)C(=O)O)CC1]

tri-tert-butyl (3R,10S,14S)-1-[(1r,4S)-4-(aminomethyl)cyclohexyl]-3-[(5-chloro-1-benzothiophen-3-yl)methyl]-1,4,12-trioxo-2,5,11,13-tetraazahexadecane-10,14,16-tricarboxylate (5.00 mg, 5.78 μmol) was solubilised in DCM (190 μl), TFA (89 μl, 1.2 mmol) was added and the mixture was stirred under argon overnight at rt. The mixture was evaporated and purified by preparative HPLC (C18, acetonitrile/water with 0.1% formic acid) to give 4.00 mg (95% purity, 94% yield) of the target compound.

LC-MS (Method 1): Rₜ=0.76 min; MS (ESIpos): m/z=697 [M+H]⁺

¹H-NMR (400 MHz, DMSO-d6) δ[ppm]: 0.828 (0.73), 0.851 (2.19), 0.888 (2.39), 0.913 (1.20), 1.082 (0.60), 1.113 (1.33), 1.146 (1.59), 1.168 (1.13), 1.197 (1.53), 1.230 (3.39), 1.249 (3.52), 1.265 (3.39), 1.310 (1.86), 1.328 (1.99), 1.345 (1.99), 1.362 (1.86), 1.378 (1.53), 1.395 (1.53), 1.414 (1.86), 1.430 (2.26), 1.446 (2.39), 1.465 (1.86), 1.497 (1.79), 1.527 (1.39), 1.601 (2.06), 1.614 (2.79), 1.634 (2.79), 1.648 (2.19), 1.669 (3.32), 1.703 (2.59), 1.760 (2.06), 1.780 (2.26), 1.794 (2.59), 1.815 (1.66), 1.831 (1.06), 1.850 (0.60), 1.904 (2.19), 2.037 (1.06), 2.067 (1.86), 2.073 (4.05), 2.097 (1.00), 2.126 (0.86), 2.139 (1.06), 2.145 (1.06), 2.162 (1.93), 2.176 (1.93), 2.181 (1.86), 2.195 (1.33), 2.216 (1.46), 2.238 (2.46), 2.252 (1.20), 2.259 (1.53), 2.274 (1.39), 2.295 (0.73), 2.332 (2.92), 2.336 (1.39), 2.352 (0.60), 2.518 (16.00), 2.523 (11.09), 2.539 (0.93), 2.552 (0.66), 2.584 (2.59), 2.599 (3.39), 2.673 (3.05), 2.678 (1.53), 2.692 (0.60), 2.710 (0.60), 2.727 (0.66), 2.888 (1.13), 2.956 (1.99), 2.972 (2.52), 2.993 (3.65), 3.004 (3.05), 3.017 (3.59), 3.030 (3.78), 3.053 (4.25), 3.077 (4.05), 3.095 (3.92), 3.110 (3.72), 3.155 (5.98), 3.168 (6.71), 3.192 (7.24), 3.204 (7.77), 3.912 (2.59), 3.932 (4.12), 3.944 (4.12), 4.483 (1.20), 4.497 (1.33), 4.506 (2.06), 4.520 (2.06), 4.528 (1.33), 4.542 (1.13), 6.140 (1.26), 6.431 (1.26), 7.362 (4.32), 7.366 (4.05), 7.383 (4.12), 7.388 (4.51), 7.533 (10.36), 7.970 (11.22), 7.974 (8.56), 7.979 (7.77), 7.991 (9.23), 8.019 (1.53), 8.250 (2.32).

#42 Linker

Intermediate 236

### methyl isoquinoline-7-carboxylate

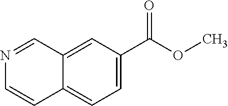

To a solution of 7-bromoisoquinoline (15.0 g, 72.1 mmol) in MeOH (150 ml) were added 1,1′-bis(diphenylphosphino)ferrocenepalladium(II) chloride (5.28 g, 7.21 mmol; CAS-RN:[72287-26-4]) and trimethylamine (20 ml, 140 mmol). The reaction mixture was stirred at 70° C. for 16 h under carbon monoxide atmosphere (50 Psi). The precipitate was removed by filtration. The filtrate was concentrated under reduced pressure. The residue was purified by flash chromatography (SiO2, petroleum ether/EtOAc gradient 10%-20%) to give 11.5 g (85% yield) of the target compound.

¹H NMR (400 MHz, CDCl₃): δ [ppm]=9.35 (s, 1H), 8.72 (s, 1H), 8.62 (dd, 1H), 8.28 (d, 1H), 7.86 (d, 1H), 7.69 (d, 1H), 4.01 (s, 3H).

Intermediate 237

### (isoquinolin-7-yl)MeOH

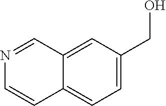 [SMILES: OCc1ccc2ccncc2c1]

To a mixture of methyl isoquinoline-7-carboxylate (8.50 g, 45.4 mmol) in THF (85 ml) was lithium aluminum hydride (1.72 g, 45.4 mmol; CAS-RN:[16853-85-3]) at 0° C. The reaction mixture was stirred at rt for 2 h. EtOAc and sodium potassium tartrate aqueous solution were added to the reaction mixture. The mixture was stirred at rt for overnight. The mixture was extracted with EtOAc. The organic phase was dried and concentrated under reduced pressure. The residue was purified by flash chromatography (SiO2, petroleum ether/EtOAc gradient 20%-50%) to give 3.90 g (54% yield) of the target compound.

Intermediate 238

### isoquinoline-7-carbaldehyde

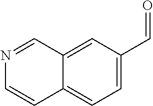 [SMILES: O=Cc1ccc2ccncc2c1]

To a solution of (isoquinolin-7-yl)MeOH (3.90 g, 24.5 mmol) in acetonitrile (50 ml) was added manganese(IV) oxide (10.6 g, 122 mmol). The reaction was stirred at 80° C. for 16 h. The solid was removed by filtration. The filtrate was concentrated under reduced pressure to give a residue. The residue was purified by flash chromatography (SiO2, petroleum ether/EtOAc gradient 20%-50%) to give 2.41 g (63% yield) of the target compound.

¹H NMR (400 MHz, CDCl₃): δ [ppm]=10.23 (s, 1H), 9.47 (s, 1H), 8.72 (d, 1H), 8.53 (s, 1H), 8.22 (dd, 1H), 7.98 (d, 1H), 7.78 (d, 1H).

Intermediate 239

### methyl (2Z)-2-{[(benzyloxy)carbonyl]amino}-3-(isoquinolin-7-yl)prop-2-enoate

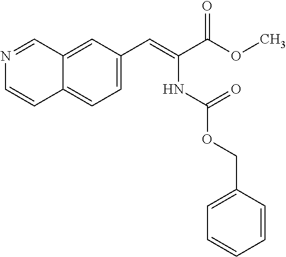 [SMILES: COC(=O)/C(=C/c1ccc2ccncc2c1)NC(=O)OCc1ccccc1]

1,8-Diazabicyclo(5.4.0)undec-7-ene (6.0 ml, 40 mmol) was added dropwise to a solution of methyl {[(benzyloxy)carbonyl]amino}(dimethoxyphosphoryl)acetate (13.4 g, 40.5 mmol) in DCM (100 ml). After stirring at rt for 10 min, a solution of isoquinoline-7-carbaldehyde (5.30 g, 33.7 mmol) in DCM (50 ml) was added. The reaction was stirred at rt for 2 h. The reaction solvent was removed under reduced pressure. The residue was diluted with EtOAc. The solution was washed with 1M HCl and brine. The organic phase was dried. The filtrate was concentrated and purified by flash chromatography (SiO2, petroleum ether/EtOAc gradient 10%-50%) to give 8.50 g (70% yield) of the target compound.

LC-MS (Method D): Rₜ=0.608 min; MS (ESIpos): m/z=363.1 [M+H]⁺.

Intermediate 240

### methyl (2R)-2-{[(benzyloxy)carbonyl]amino}-3-(isoquinolin-7-yl)propanoate

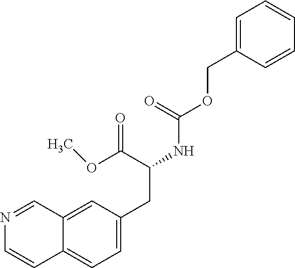 [SMILES: COC(=O)[C@@H](Cc1ccc2ccncc2c1)NC(=O)OCc1ccccc1]

To a solution of methyl (2Z)-2-{[(benzyloxy)carbonyl]amino}-3-(isoquinolin-7-yl)prop-2-enoate (3.20 g, 8.83 mmol) in MeOH were added (R)—[Rh(COD)(MaxPHOS)]BF4 (150 ml). The reaction mixture was stirred at rt for 96 h under hydrogen atmosphere (1 MPa). The reaction mixture was evaporated under reduced pressure. The residue was purified by flash chromatography (SiO2, petroleum ether/EtOAc gradient 10%-50%) to give 1.20 g (91% purity, 34% yield) and 500 mg (50% purity, 8% yield) of the target compound.

LC-MS (Method D): Rₜ=0.623 min; MS (ESIpos): m/z=365.0 [M+H]⁺.

Intermediate 241

### (2R)-2-{[(benzyloxy)carbonyl]amino}-3-(isoquinolin-7-yl)propanoic acid

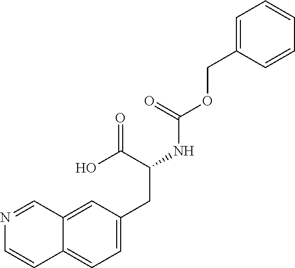 [SMILES: O=C(N[C@H](Cc1ccc2ccncc2c1)C(=O)O)OCc1ccccc1]

To a cooled solution (0° C.) of methyl (2R)-2-{[(benzyloxy)carbonyl]amino}-3-(isoquinolin-7-yl)propanoate (3.30 g, 9.06 mmol) in THF (30 ml) was added lithium hydroxide (5.4 ml, 2.0 M, 11 mmol; CAS-RN:[1310-65-2]). The reaction mixture was stirred at rt for 16 h. The reaction solution was concentrated under reduced pressure and dissolved in MeOH. The pH of the mixture was adjusted to 5 with formic acid. The mixture was concentrated and purified by preparative HPLC (C18, acetonitrile/water with 0.1% formic acid) to give 2.00 g (63% yield) of the target compound.

LC-MS (Method C): Rₜ=0.685 min; MS (ESIpos): m/z=351.1 [M+H]⁺.

¹H-NMR (400 MHz, DMSO-d₆): δ [ppm]=9.23 (s, 1H), 8.48 (d, 1H), 7.96 (s, 1H), 7.90 (d, 1H), 7.83-7.68 (m, 3H), 7.29-7.07 (m, 5H), 4.94 (s, 2H), 4.39-4.31 (m, 1H), 3.27-3.22 (m, 1H), 3.07 (dd, 1H).

Intermediate 242

### tri-tert-butyl (5R,12S,16S)-5-[(isoquinolin-7-yl)methyl]-3,6,14-trioxo-1-phenyl-2-oxa-4,7,13,15-tetraazaoctadecane-12,16,18-tricarboxylate

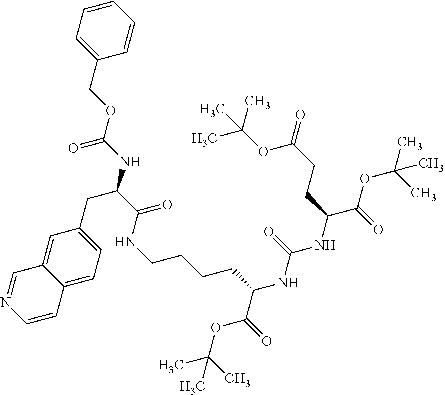 [SMILES: CC(C)(C)OC(=O)CC[C@H](NC(=O)N[C@@H](CCCCNC(=O)[C@@H](Cc1ccc2ccncc2c1)NC(=O)OCc1ccccc1)C(=O)OC(C)(C)C)C(=O)OC(C)(C)C]

di-tert-butyl N-{[(2S)-6-amino-1-tert-butoxy-1-oxohexan-2-yl]carbamoyl}-L-glutamate (1.11 g, 2.28 mmol) and (2R)-2-{[(benzyloxy)carbonyl]amino}-3-(isoquinolin-7-yl)propanoic acid (800 mg, 2.28 mmol) were solubilised in DMF (18 ml), 4-methylmorpholine (630 μl, 5.7 mmol, CAS-RN: 109-02-4) and HATU (1.22 g, 3.20 mmol) were added and the mixture was stirred under argon overnight at rt. The mixture was evaporated and purified by preparative HPLC (C18, acetonitrile/water with 0.1% formic acid) to give 460 mg (98% purity, 24% yield) of the target compound.

LC-MS (Method 1): Rₜ=1.28 min; MS (ESIpos): m/z=821 [M+H]⁺

¹H-NMR (400 MHz, DMSO-d6) δ[ppm]: 1.107 (10.01), 1.381 (16.00), 1.384 (15.19), 2.518 (1.60), 2.522 (1.00), 2.539 (0.89), 4.190 (0.78), 4.885 (0.48), 4.908 (0.53), 7.155 (0.46), 7.159 (0.45), 7.210 (0.53), 7.226 (0.63), 7.795 (0.42), 7.868 (0.41), 7.925 (0.42), 8.453 (0.82), 8.468 (0.71), 9.203 (0.73).

Intermediate 243

### di-tert-butyl (2S)-2-({[(2S)-6-{[(2R)-2-amino-3-(isoquinolin-7-yl)propanoyl]amino}-1-tert-butoxy-1-oxohexan-2-yl]carbamoyl}amino)pentanedioate

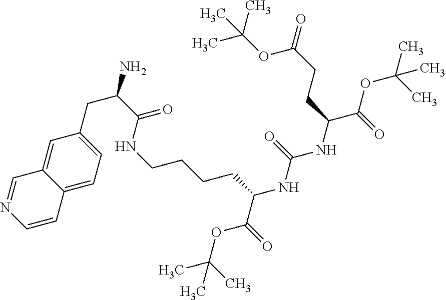 [SMILES: CC(C)(C)OC(=O)CC[C@H](NC(=O)N[C@@H](CCCCNC(=O)[C@H](N)Cc1ccc2ccncc2c1)C(=O)OC(C)(C)C)C(=O)OC(C)(C)C]

tri-tert-butyl (5R,12S,16S)-5-[(isoquinolin-7-yl)methyl]-3,6,14-trioxo-1-phenyl-2-oxa-4,7,13,15-tetraazaoctadecane-12,16,18-tricarboxylate (460 mg, 561 μmol) was solubilised in MeOH (4.5 ml), Palladium on carbon (59.7 mg, 10% purity, 56.1 μmol) was added and the mixture was purged with hydrogen. The mixture was stirred for 8 h at rt under hydrogen atmosphere. The mixture was filtered over Celite, washed with MeOH and evaporated to give 330 mg (91% purity, 78% yield) of the target compound.

LC-MS (Method 1): Rₜ=1.29 min; MS (ESIpos): m/z=686 [M+H]⁺

Intermediate 244

### tri-tert-butyl (3R,10S,14S)-1-{(1r,4S)-4-[({[(9H-fluoren-9-yl)methoxy]carbonyl}amino)methyl]cyclohexyl}-3-[(isoquinolin-7-yl)methyl]-1,4,12-trioxo-2,5,11,13-tetraazahexadecane-10,14,16-tricarboxylate

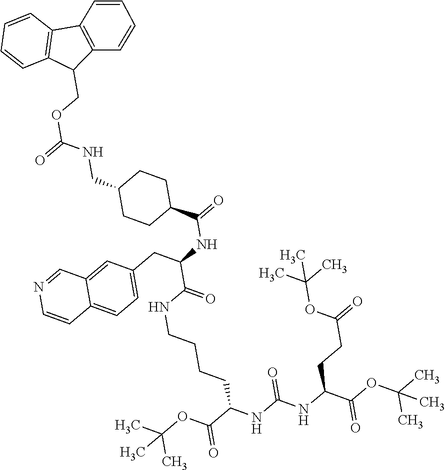 [SMILES: CC(C)(C)OC(=O)CC[C@H](NC(=O)N[C@@H](CCCCNC(=O)[C@@H](Cc1ccc2ccncc2c1)NC(=O)[C@H]1CC[C@H](CNC(=O)OCC2c3ccccc3-c3ccccc32)CC1)C(=O)OC(C)(C)C)C(=O)OC(C)(C)C]

(1r,4r)-4-[({[(9H-fluoren-9-yl)methoxy]carbonyl}amino)methyl]cyclohexane-1-carboxylic acid (183 mg, 482 μmol) was solubilised in DMF (6.7 ml), 4-methylmorpholine (140 μl, 1.3 mmol, CAS-RN: 109-02-4) and HATU (183 mg, 482 μmol) were added, stirred for 20 min at rt, di-tert-butyl (2S)-2-({[(2S)-6-{[(2R)-2-amino-3-(isoquinolin-7-yl)propanoyl]amino}-1-tert-butoxy-1-oxohexan-2-yl]carbamoyl}amino)pentanedioate (330 mg, 91% purity, 438 μmol) was added and the mixture was stirred under argon overnight at rt. The mixture was filtered and purified by preparative HPLC (C18, acetonitrile/water with 0.1% formic acid) to give 150 mg (95% purity, 31% yield) of the target compound.

LC-MS (Method 1): Rₜ=1.42 min; MS (ESIpos): m/z=1049 [M+H]⁺

Intermediate 245

### tri-tert-butyl (3R,10S,14S)-1-[(1r,4S)-4-(aminomethyl)cyclohexyl]-3-[(isoquinolin-7-yl)methyl]-1,4,12-trioxo-2,5,11,13-tetraazahexadecane-10,14,16-tricarboxylate

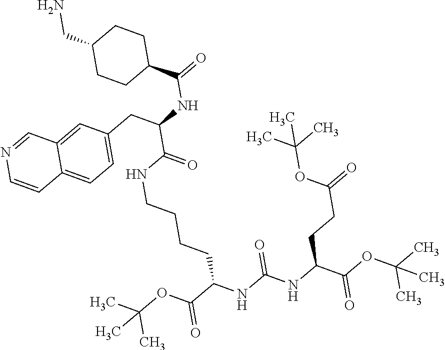 [SMILES: CC(C)(C)OC(=O)CC[C@H](NC(=O)N[C@@H](CCCCNC(=O)[C@@H](Cc1ccc2ccncc2c1)NC(=O)[C@H]1CC[C@H](CN)CC1)C(=O)OC(C)(C)C)C(=O)OC(C)(C)C]

tri-tert-butyl (3R,10S,14S)-1-{(1r,4S)-4-[({[(9H-fluoren-9-yl)methoxy]carbonyl}amino)methyl]cyclohexyl}-3-[(isoquinolin-7-yl)methyl]-1,4,12-trioxo-2,5,11,13-tetraazahexadecane-10,14,16-tricarboxylate (150 mg, 95% purity, 136 μmol) was solubilised in DMF (2.1 ml), piperidine (270 μl, 2.7 mmol) was added and the mixture was stirred under argon overnight at rt. The mixture was evaporated and purified by preparative HPLC (C18, acetonitrile/water with 0.1% formic acid) to give 84.0 mg (98% purity, 73% yield) of the target compound.

LC-MS (Method 1): Rₜ=0.96 min; MS (ESIpos): m/z=826 [M+H]⁺

¹H-NMR (400 MHz, DMSO-d6) δ[ppm]: 1.381 (12.36), 1.387 (16.00), 2.518 (1.73), 2.522 (1.09), 7.872 (0.71), 8.419 (0.59), 8.431 (0.59), 8.446 (0.52), 9.190 (0.61).

### Example 42-A

### (3R,10S,14S)-1-[(1r,4S)-4-(aminomethyl)cyclohexyl]-3-[(isoquinolin-7-yl)methyl]-1,4,12-trioxo-2,5,11,13-tetraazahexadecane-10,14,16-tricarboxylic acid

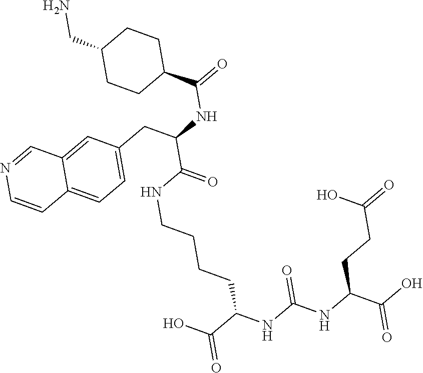 [SMILES: NC[C@H]1CC[C@H](C(=O)N[C@H](Cc2ccc3ccncc3c2)C(=O)NCCCC[C@H](NC(=O)N[C@@H](CCC(=O)O)C(=O)O)C(=O)O)CC1]

tri-tert-butyl (3R,10S,14S)-1-[(1r,4S)-4-(aminomethyl)cyclohexyl]-3-[(isoquinolin-7-yl)methyl]-1,4,12-trioxo-2,5,11,13-tetraazahexadecane-10,14,16-tricarboxylate (10.0 mg, 98% purity, 11.9 μmol) was solubilised in DCM (380 μl), TFA (180 μl, 2.4 mmol) was added and the mixture was stirred under argon overnight at rt. The mixture was evaporated and purified by preparative HPLC (C18, acetonitrile/water with 0.1% formic acid) to give 4.00 mg (95% purity, 49% yield) of the target compound.

LC-MS (Method 1): Rₜ=0.45 min; MS (ESIpos): m/z=658 [M+H]⁺

¹H-NMR (400 MHz, DMSO-d6) δ[ppm]: 0.832 (2.25), 0.862 (2.50), 0.895 (1.07), 0.987 (0.61), 1.018 (1.38), 1.049 (1.28), 1.076 (0.51), 1.142 (0.61), 1.175 (1.38), 1.207 (1.74), 1.231 (2.96), 1.248 (3.17), 1.266 (3.42), 1.302 (1.79), 1.319 (1.84), 1.339 (1.84), 1.354 (2.15), 1.374 (2.20), 1.412 (2.56), 1.431 (3.32), 1.624 (3.42), 1.645 (4.55), 1.732 (1.53), 1.762 (1.79), 1.799 (1.48), 1.817 (1.28), 1.834 (0.97), 2.029 (1.12), 2.058 (1.84), 2.073 (2.15), 2.133 (0.77), 2.147 (1.02), 2.169 (1.79), 2.183 (1.79), 2.201 (1.23), 2.222 (1.33), 2.244 (2.25), 2.265 (1.38), 2.279 (1.28), 2.301 (0.72), 2.322 (2.45), 2.326 (3.17), 2.331 (2.40), 2.522 (9.66), 2.572 (3.83), 2.587 (3.99), 2.664 (2.40), 2.669 (3.22), 2.673 (2.40), 2.944 (2.35), 2.977 (3.99), 3.002 (3.78), 3.059 (2.91), 3.074 (3.32), 3.091 (3.12), 3.108 (2.91), 3.154 (4.81), 3.166 (5.32), 3.188 (5.62), 3.200 (5.83), 3.364 (16.00), 3.938 (3.78), 3.949 (3.68), 4.503 (1.07), 4.516 (1.28), 4.528 (1.84), 4.538 (1.89), 4.550 (1.23), 4.562 (1.02), 6.154 (1.28), 6.425 (1.38), 7.658 (2.96), 7.662 (3.07), 7.679 (3.58), 7.683 (3.58), 7.758 (3.37), 7.772 (3.42), 7.847 (4.91), 7.868 (3.99), 7.895 (5.98), 7.967 (1.74), 8.238 (1.48), 8.429 (3.17), 8.443 (3.02), 9.192 (4.60).

Intermediate 246

### tri-tert-butyl (3R,10S,14S)-3-[(isoquinolin-7-yl)methyl]-1,4,12-trioxo-1-[(1r,4S)-4-({2-[4,7,10-tris(2-tert-butoxy-2-oxoethyl)-1,4,7,10-tetraazacyclododecan-1-yl]acetamido}methyl)cyclohexyl]-2,5,11,13-tetraazahexadecane-10,14,16-tricarboxylate

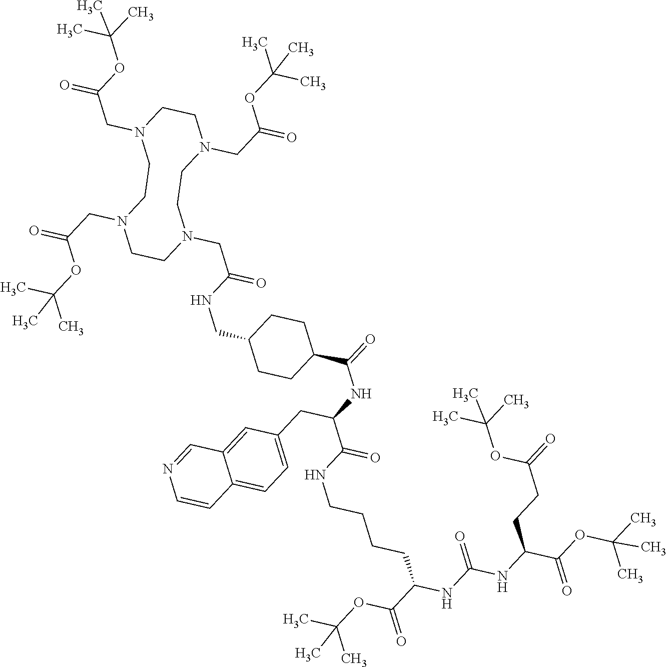 [SMILES: CC(C)(C)OC(=O)CC[C@H](NC(=O)N[C@@H](CCCCNC(=O)[C@@H](Cc1ccc2ccncc2c1)NC(=O)[C@H]1CC[C@H](CNC(=O)CN2CCN(CC(=O)OC(C)(C)C)CCN(CC(=O)OC(C)(C)C)CCN(CC(=O)OC(C)(C)C)CC2)CC1)C(=O)OC(C)(C)C)C(=O)OC(C)(C)C]

[4,7,10-tris(2-tert-butoxy-2-oxoethyl)-1,4,7,10-tetraazacyclododecan-1-yl]acetic acid (136 mg, 238 μmol; CAS-RN:[137076-54-1]), [(1H-benzotriazol-1-yl)oxy](dimethylamino)-N,N-dimethylmethaniminium hexafluoridophosphate(1−) (89.0 mg, 235 μmol; CAS-RN:94790-37-1) and N,N-diisopropylethylamine (30 μl, 180 μmol) were stirred in DMF (6.2 ml) for 10 min at rt. tri-tert-butyl (3R,10S,14S)-1-[(1r,4S)-4-(aminomethyl)cyclohexyl]-3-[(isoquinolin-7-yl)methyl]-1,4,12-trioxo-2,5,11,13-tetraazahexadecane-10,14,16-tricarboxylate (49.0 mg, 59.4 μmol) was added and the mixture was stirred overnight at rt. The mixture was evaporated and purified by preparative HPLC (C18, acetonitrile/water with 0.1% formic acid) to give 70.0 mg (95% purity, 81% yield) of the target compound.

LC-MS (Method 1): Rₜ=1.26 min; MS (ESIpos): m/z=1381 [M+H]⁺

### Example 42-B

### (3R,10S,14S)-3-[(isoquinolin-7-yl)methyl]-1,4,12-trioxo-1-[(1r,4S)-4-({2-[4,7,10-tris(carboxymethyl)-1,4,7,10-tetraazacyclododecan-1-yl]acetamido}methyl)cyclohexyl]-2,5,11,13-tetraazahexadecane-10,14,16-tricarboxylic acid

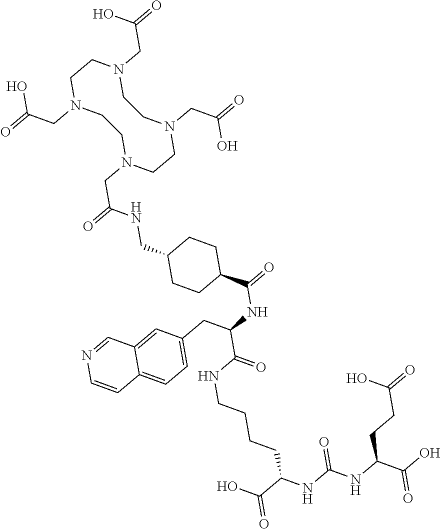 [SMILES: O=C(O)CC[C@H](NC(=O)N[C@@H](CCCCNC(=O)[C@@H](Cc1ccc2ccncc2c1)NC(=O)[C@H]1CC[C@H](CNC(=O)CN2CCN(CC(=O)O)CCN(CC(=O)O)CCN(CC(=O)O)CC2)CC1)C(=O)O)C(=O)O]

tri-tert-butyl (3R,10S,14S)-3-[(isoquinolin-7-yl)methyl]-1,4,12-trioxo-1-[(1r,4S)-4-({2-[4,7,10-tris(2-tert-butoxy-2-oxoethyl)-1,4,7,10-tetraazacyclododecan-1-yl]acetamido}methyl)cyclohexyl]-2,5,11,13-tetraazahexadecane-10,14,16-tricarboxylate (4.00 mg, 2.90 μmol) was solubilised in DCM (1.0 ml), TFA (510 μl) was added and the mixture was stirred under argon overnight at rt. The mixture was evaporated and purified by preparative HPLC (C18, acetonitrile/water with 0.1% formic acid) to give 2.50 mg (90% purity, 74% yield) of the target compound.

LC-MS (Method 1): Rₜ=0.48 min; MS (ESIpos): m/z=1042 [M−H]⁻

¹H-NMR (400 MHz, DMSO-d6) δ[ppm]: 0.788 (0.96), 0.818 (1.08), 0.852 (1.02), 1.044 (0.45), 1.075 (0.45), 1.233 (3.00), 1.296 (1.27), 1.316 (1.53), 1.333 (1.47), 1.352 (1.21), 1.438 (1.34), 1.453 (1.21), 1.592 (1.59), 1.685 (0.76), 1.705 (0.64), 1.726 (0.76), 1.741 (0.57), 1.881 (0.70), 1.899 (0.70), 1.986 (0.51), 2.005 (0.76), 2.038 (0.83), 2.074 (0.70), 2.212 (1.08), 2.227 (1.53), 2.245 (1.53), 2.263 (0.96), 2.322 (2.87), 2.327 (3.82), 2.332 (2.87), 2.518 (16.00), 2.522 (10.07), 2.642 (2.10), 2.660 (2.61), 2.664 (3.82), 2.669 (4.65), 2.673 (3.44), 2.895 (1.91), 2.980 (5.16), 3.005 (5.04), 3.088 (2.29), 3.140 (1.53), 3.152 (1.72), 3.173 (1.72), 3.185 (1.72), 3.334 (10.01), 3.958 (0.51), 3.979 (1.02), 3.991 (1.02), 4.011 (0.51), 4.052 (0.51), 4.071 (0.96), 4.084 (0.96), 4.104 (0.45), 4.488 (0.45), 4.511 (0.76), 4.523 (0.83), 4.547 (0.45), 6.300 (1.47), 6.319 (1.66), 6.332 (1.15), 7.656 (1.40), 7.678 (1.66), 7.758 (2.10), 7.772 (2.23), 7.851 (2.49), 7.873 (2.04), 7.888 (2.68), 7.979 (0.89), 8.072 (1.47), 8.138 (2.49), 8.428 (4.08), 8.442 (3.63), 9.202 (4.53).

### Example 42-B 227Th

### (3R,10S,14S)-3-[(isoquinolin-7-yl)methyl]-1,4,12-trioxo-1-{(1r,4S)-4-[(2-{4,7,10-tris[(carboxy-kappaO)methyl]-1,4,7,10-tetraazacyclododecan-1-yl-kappa²N¹,N⁴}acetamido)methyl]cyclohexyl}-2,5,11,13-tetraazahexadecane-10,14,16-tricarboxylato(3−)(²²⁷Th)Thorium

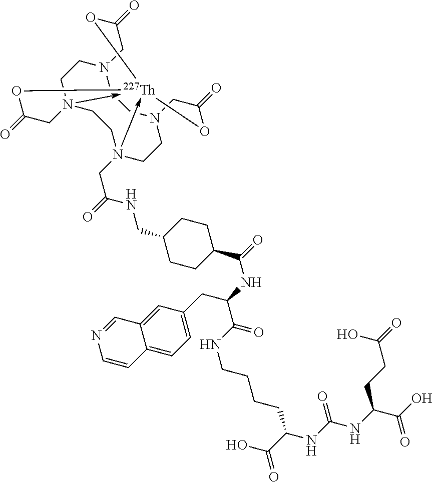

(3R,10S,14S)-3-[(isoquinolin-7-yl)methyl]-1,4,12-trioxo-1-[(1r,4S)-4-({2-[4,7,10-tris(carboxymethyl)-1,4,7,10-tetraazacyclododecan-1-yl]acetamido}methyl)cyclohexyl]-2,5,11,13-tetraazahexadecane-10,14,16-tricarboxylic acid was dissolved as 10 mM solution in DMSO and diluted to a 250 μM solution in 400 mM sodium acetate buffer (pH 5) containing 0.5 mg/mL pABA. 1.07 μl of this solution was used in a 40 μl labeling reaction. Thorium-227 in 400 mM sodium acetate buffer (pH 5) plus 20% EtOH was added giving a specific activity of 0.375 MBq/nmol and a RAC of 2.5 MBq/mL. The mixture was incubated at 95° C. for 60 min. The labelling efficiency was determined to be 96.1% by iTLC.

#43 Linker

Intermediate 247

### tri-tert-butyl (3S,10S,14S)-3-[(1-benzothiophen-3-yl)methyl]-1-{(1r,4S)-4-[({[(9H-fluoren-9-yl)methoxy]carbonyl}amino)methyl]cyclohexyl}-1,4,12-trioxo-2,5,11,13-tetraazahexadecane-10,14,16-tricarboxylate

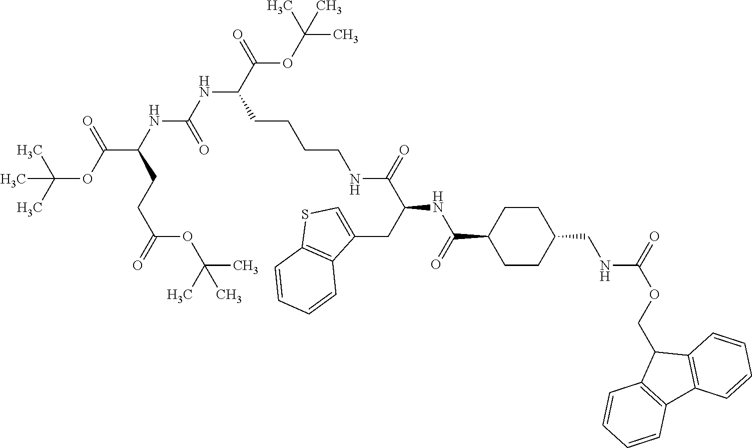 [SMILES: CC(C)(C)OC(=O)CC[C@H](NC(=O)N[C@@H](CCCCNC(=O)[C@H](Cc1csc2ccccc12)NC(=O)[C@H]1CC[C@H](CNC(=O)OCC2c3ccccc3-c3ccccc32)CC1)C(=O)OC(C)(C)C)C(=O)OC(C)(C)C]

(1 r,4r)-4-[({[(9H-fluoren-9-yl)methoxy]carbonyl}amino)methyl]cyclohexane-1-carboxylic acid (57.4 mg, 151 μmol) was solubilised in DMF (2.1 ml), 4-methylmorpholine (45 μl, 410 μmol, CAS-RN: 109-02-4) and HATU (57.5 mg, 151 μmol) were added, stirred for 20 min at rt, di-tert-butyl (2S)-2-({[(2S)-6-{[(2S)-2-amino-3-(1-benzothiophen-3-yl)propanoyl]amino}-1-tert-butoxy-1-oxohexan-2-yl]carbamoyl}amino)pentanedioate (95.0 mg, 138 μmol) was added and the mixture was stirred under argon overnight at rt. The mixture was filtered and purified by preparative HPLC (C18, acetonitrile/water with 0.1% formic acid) to give 40.0 mg (95% purity, 26% yield) of the target compound.

LC-MS (Method 1): Rₜ=1.64 min; MS (ESIpos): m/z=1053 [M+H]⁺

Intermediate 248

### tri-tert-butyl (3S,10S,14S)-1-[(1r,4S)-4-(aminomethyl)cyclohexyl]-3-[(1-benzothiophen-3-yl)methyl]-1,4,12-trioxo-2,5,11,13-tetraazahexadecane-10,14,16-tricarboxylate

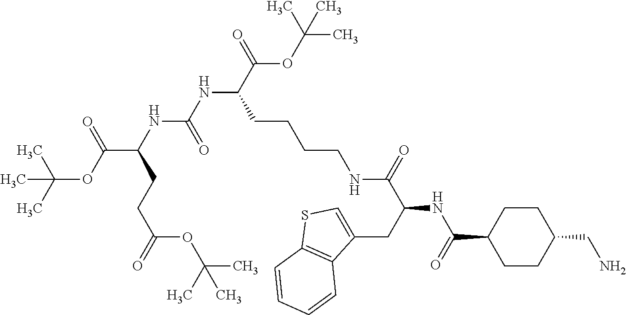 [SMILES: CC(C)(C)OC(=O)CC[C@H](NC(=O)N[C@@H](CCCCNC(=O)[C@H](Cc1csc2ccccc12)NC(=O)[C@H]1CC[C@H](CN)CC1)C(=O)OC(C)(C)C)C(=O)OC(C)(C)C]

tri-tert-butyl (3S,10S,14S)-3-[(1-benzothiophen-3-yl)methyl]-1-{(1r,4S)-4-[({[(9H-fluoren-9-yl)methoxy]carbonyl}amino)methyl]cyclohexyl}-1,4,12-trioxo-2,5,11,13-tetraazahexadecane-10,14,16-tricarboxylate (40.0 mg, 38.0 μmol) was solubilised in DMF (580 μl), piperidine (75 μl, 760 μmol) was added and the mixture was stirred under argon for 2 h at rt. The mixture was evaporated and purified by preparative HPLC (C18, acetonitrile/water with 0.1% formic acid) to give 27.0 mg (96% purity, 82% yield) of the target compound.

LC-MS (Method 1): Rₜ=1.20 min; MS (ESIpos): m/z=831 [M+H]⁺

¹H-NMR (400 MHz, DMSO-d6) δ[ppm]: 1.370 (0.78), 1.383 (16.00), 1.387 (11.91), 1.391 (11.33), 2.518 (1.65), 2.522 (0.95), 7.374 (0.86), 8.429 (0.47).

### Example 43-A

### N⁶—{N-[(1r,4S)-4-(aminomethyl)cyclohexane-1-carbonyl]-3-(1-benzothiophen-3-yl)-L-alanyl}-N²-{[(1S)-1,3-dicarboxypropyl]carbamoyl}-L-lysine

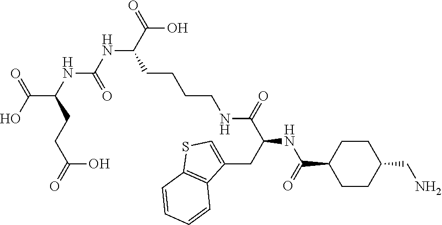 [SMILES: NC[C@H]1CC[C@H](C(=O)N[C@@H](Cc2csc3ccccc23)C(=O)NCCCC[C@H](NC(=O)N[C@@H](CCC(=O)O)C(=O)O)C(=O)O)CC1]

tri-tert-butyl (3S,10S,14S)-1-[(1r,4S)-4-(aminomethyl)cyclohexyl]-3-[(1-benzothiophen-3-yl)methyl]-1,4,12-trioxo-2,5,11,13-tetraazahexadecane-10,14,16-tricarboxylate (11.6 mg, 14.0 μmol) was solubilised in DCM (450 μl), TFA (220 μl, 2.8 mmol) was added and the mixture was stirred under argon overnight at rt. The mixture was evaporated and purified by preparative HPLC (C18, acetonitrile/water with 0.1% formic acid) to give 7.10 mg (95% purity, 73% yield) of the target compound.

LC-MS (Method 1): Rₜ=0.70 min; MS (ESIpos): m/z=663 [M+H]⁺

¹H-NMR (400 MHz, DMSO-d6) δ[ppm]: 0.851 (3.63), 0.884 (3.48), 0.911 (1.72), 1.066 (0.86), 1.083 (1.16), 1.131 (3.13), 1.149 (3.68), 1.208 (2.83), 1.232 (8.18), 1.246 (6.71), 1.255 (6.46), 1.268 (5.30), 1.295 (3.79), 1.315 (3.13), 1.331 (7.27), 1.352 (3.28), 1.373 (2.37), 1.382 (2.42), 1.389 (2.37), 1.433 (3.38), 1.451 (3.48), 1.470 (2.78), 1.517 (2.47), 1.549 (2.22), 1.613 (2.57), 1.632 (2.68), 1.687 (5.50), 1.702 (4.74), 1.761 (4.14), 1.780 (3.94), 1.795 (3.48), 1.988 (0.45), 2.005 (0.45), 2.046 (1.46), 2.076 (2.47), 2.106 (1.41), 2.157 (1.36), 2.178 (2.68), 2.198 (3.28), 2.226 (3.53), 2.247 (2.07), 2.263 (1.46), 2.285 (0.86), 2.297 (2.12), 2.323 (2.47), 2.326 (3.18), 2.332 (2.47), 2.539 (1.77), 2.609 (4.59), 2.664 (2.52), 2.669 (3.23), 2.673 (2.47), 2.728 (8.73), 2.817 (1.06), 2.888 (10.40), 2.975 (3.38), 3.002 (4.64), 3.025 (4.19), 3.038 (4.90), 3.062 (5.20), 3.086 (3.74), 3.103 (3.23), 3.181 (5.96), 3.193 (6.56), 3.216 (6.51), 3.230 (6.66), 3.385 (9.89), 3.578 (3.99), 3.946 (5.45), 3.960 (5.25), 4.519 (1.46), 4.541 (2.57), 4.554 (2.62), 4.576 (1.36), 6.174 (1.92), 6.412 (2.27), 6.430 (2.22), 7.126 (0.40), 7.160 (0.81), 7.180 (0.91), 7.207 (0.76), 7.230 (0.40), 7.254 (0.76), 7.273 (0.76), 7.335 (2.12), 7.352 (4.74), 7.370 (4.04), 7.386 (4.14), 7.402 (16.00), 7.420 (2.32), 7.889 (5.25), 7.908 (4.79), 7.932 (6.16), 7.951 (7.92), 7.970 (3.13), 8.189 (2.17), 8.211 (2.27), 8.227 (2.52).

#44 Linker

Intermediate 249

### methyl isoquinoline-6-carboxylate

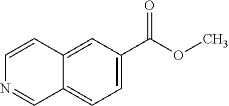

To a solution of isoquinoline-6-carboxylic acid (8.50 g, 49.1 mmol) in MeOH (100 ml) was added thionyl chloride (11 ml, 150 mmol) at rt. The reaction mixture was stirred at 65° C. for 12 h. The mixture was concentrated under reduced pressure. The residue was purified by flash chromatography (SiO2, petroleum ether/EtOAc gradient 5%-33%) to give 8.40 g (91% yield) of the target compound.

¹H NMR (400 MHz, CDCl₃): δ [ppm]=9.33 (s, 1H), 8.61 (d, 1H), 8.57 (s, 1H), 8.18 (d, 1H), 8.03 (d, 1H), 7.75 (d, 1H), 4.01 (s, 3H).

Intermediate 250

### (isoquinolin-6-yl)MeOH

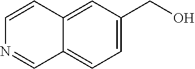 [SMILES: OCc1ccc2cnccc2c1]

To a solution of methyl isoquinoline-6-carboxylate (8.40 g, 44.9 mmol) in THF (150 ml) was added lithium aluminum hydride (2.2 ml, 10 M, 22 mmol; CAS-RN:[16853-85-3]) at 0° C. The reaction mixture was stirred at 25° C. for 1 h. The mixture was quenched by saturated ammonium chloride and extracted with EtOAc. The organic phase was washed with brine, dried and concentrated under reduced pressure. The residue was purified by flash chromatography (SiO2, petroleum ether/EtOAc gradient 10%-50%) to give 6.40 g (90% yield) of the target compound.

¹H NMR (400 MHz, DMSO-d₆): δ [ppm]=9.26 (s, 1H), 8.47 (d, 1H), 8.06 (d, 1H), 7.85 (s 1H), 7.80 (d, 1H), 7.60 (d, 1H), 5.49 (t, 1H), 4.72 (d, 2H).

Intermediate 251

### isoquinoline-6-carbaldehyde

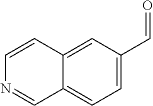 [SMILES: O=Cc1ccc2cnccc2c1]

To a solution of (isoquinolin-6-yl)MeOH (6.40 g, 40.2 mmol) in DCM (130 ml) was added 3,3,3-triacetoxy-3-iodophthalide (22.2 g, 52.3 mmol; CAS-RN:[87413-09-0]) at rt. The reaction mixture was stirred at 25° C. for 1 h. The mixture was filtered through a pad of celite and the filtrate was concentrated to give a residue. The residue was purified by flash chromatography (SiO2, petroleum ether/EtOAc gradient 10%-50%) to give 6.70 g (90% purity, 95% yield) of the target compound.

¹H NMR (400 MHz, DMSO-d₆): δ [ppm]=10.22 (s, 1H), 9.46 (s, 1H), 8.66 (d, 1H), 8.62 (s, 1H), 8.28 (d, 1H), 8.08-8.04 (m, 2H).

Intermediate 252 methyl (2Z)-2-{[(benzyloxy)carbonyl]amino}-3-(isoquinolin-6-yl)prop-2-enoate

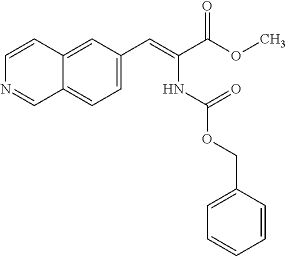 [SMILES: COC(=O)/C(=C/c1ccc2cnccc2c1)NC(=O)OCc1ccccc1]

To a solution of methyl (2S)-{[(benzyloxy)carbonyl]amino}(dimethoxyphosphoryl)acetate (16.5 g, 49.9 mmol) in DCM (100 ml) was added 1,8-diazabicyclo(5.4.0)undec-7-ene (8.6 ml, 58 mmol) at rt. After stirred for 0.5 h, a solution of isoquinoline-6-carbaldehyde (6.70 g, 90% purity, 38.4 mmol) in DCM (50 ml) was added and the reaction mixture was stirred at rt for 2 h. The mixture was quenched with saturated ammonium chloride and extracted with EtOAc. The organic phase was washed with brine, dried and concentrated to give a residue. The residue was purified by flash chromatography (SiO2, petroleum ether/EtOAc gradient 10%-33%) to give 2.80 g (96% purity, 21% yield) and 1.20 g (87% purity, 8% yield) of the target compound.

LC-MS (Method D): Rₜ=0.600 min; MS (ESIpos): m/z=363.1 [M+H]⁺.

¹H NMR (400 MHz, DMSO-d₆): δ [ppm]=9.40 (br.s, 1H), 9.31 (s, 1H), 8.53 (d, 1H), 8.18 (s, 1H), 8.11 (d, 1H), 7.92 (d, 1H), 7.75 (d, 1H), 7.45-7.30 (m, 6H), 5.12 (s, 2H), 3.75 (s, 3H).

Intermediate 253

### methyl (2R)-2-{[(benzyloxy)carbonyl]amino}-3-(isoquinolin-6-yl)propanoate

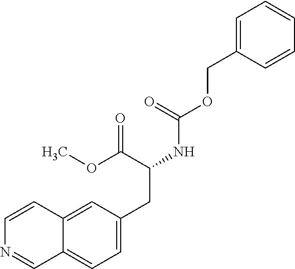 [SMILES: COC(=O)[C@@H](Cc1ccc2cnccc2c1)NC(=O)OCc1ccccc1]

To a solution of methyl (2Z)-2-{[(benzyloxy)carbonyl]amino}-3-(isoquinolin-6-yl)prop-2-enoate (2.80 g, 96% purity, 7.42 mmol) in methanol (100 ml) were added (R)—[Rh(COD)(MaxPhos)]BF₄ (220 mg, 0.371 mmol). The reaction mixture was stirred at room temperature for 96 hours under hydrogen atmosphere (2.5 MPa). The mixture was concentrated and filtered through silica gel (200-300 mesh). The filtrate was concentrated and purified by preparative HPLC (Instrument: Shimadzu LC-20AP; Column: Phenomenex Synergi Max-RP 250*50 mm*10 μm; eluent A: 0.225% formic acid in water, eluent B: acetonitrile; gradient: 0-30 min 5-40% B; flow 100 ml/min; temperature: room temperature; Detector: UV 220/254 nm) to give methyl (2S)-2-{[(benzyloxy)carbonyl]amino}-3-(isoquinolin-6-yl)propanoate (1.10 g, 41% yield) as yellow oil and methyl (2Z)-2-{[(benzyloxy)carbonyl]amino}-3-(isoquinolin-6-yl)acrylate (0.8 g) as yellow oil.

Intermediate 254

### (2R)-2-{[(benzyloxy)carbonyl]amino}-3-(isoquinolin-6-yl)propanoic acid

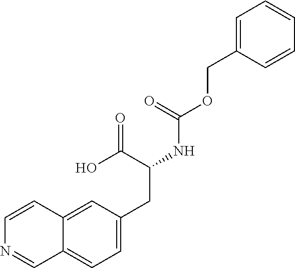 [SMILES: O=C(N[C@H](Cc1ccc2cnccc2c1)C(=O)O)OCc1ccccc1]

To a solution of methyl (2R)-2-{[(benzyloxy)carbonyl]amino}-3-(isoquinolin-6-yl)propanoate (1.10 g, 3.02 mmol) in pyridine (20 ml) was added lithium iodide anhydrous (1.21 g, 9.06 mmol) at room temperature. The reaction mixture was stirred at 105° C. for 12 hours. The mixture (combined with another batch starting with 0.80 g methylester) was concentrated and purified by preparative HPLC (Instrument: Shimadzu LC-20AP; Column: Phenomenex Synergi Max-RP 250*50 mm*10 μm; eluent A: 0.225% formic acid in water, eluent B: acetonitrile; gradient: 0-24 min 8-38% B; flow 100 ml/min; temperature: room temperature; Detector: UV 220/254 nm) to give (2S)-2-{[(benzyloxy)carbonyl]amino}-3-(isoquinolin-6-yl)propanoic acid as a brown solid. The product (combined with another batch starting with 0.80 g methylester) was dissolved in a mixed solvent of acetonitrile and water, lyophilized to give (2S)-2-{[(benzyloxy)carbonyl]amino}-3-(isoquinolin-6-yl)propanoic acid (1.17 g) a brown solid.

LC-MS (Method C): Rₜ=0.636 min; MS (ESIpos): m/z=351.0 [M+H]⁺.

¹H NMR (400 MHz, DMSO-d₆): δ [ppm]=9.51 (s, 1H), 8.55 (d, 1H), 8.21 (d, 1H), 8.02 (d, 1H), 7.95 (s, 1H), 7.77 (d, 1H), 7.26-7.19 (m, 5H), 4.93 (s, 2H), 4.42-4.36 (m, 1H), 3.38-3.34 (m, 1H), 3.14-3.08 (m, 1H).

Intermediate 255

### tri-tert-butyl (5R,12S,16S)-5-[(isoquinolin-6-yl)methyl]-3,6,14-trioxo-1-phenyl-2-oxa-4,7,13,15-tetraazaoctadecane-12,16,18-tricarboxylate

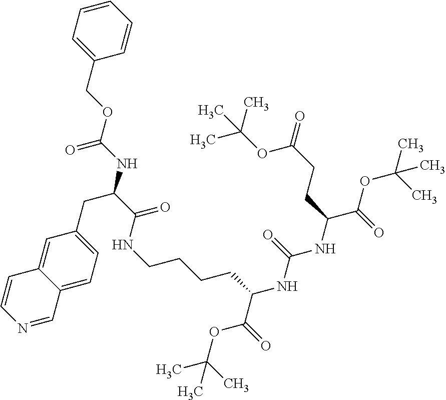 [SMILES: CC(C)(C)OC(=O)CC[C@H](NC(=O)N[C@@H](CCCCNC(=O)[C@@H](Cc1ccc2cnccc2c1)NC(=O)OCc1ccccc1)C(=O)OC(C)(C)C)C(=O)OC(C)(C)C]

di-tert-butyl N-{[(2S)-6-amino-1-tert-butoxy-1-oxohexan-2-yl]carbamoyl}-L-glutamate (660 mg, 1.35 mmol) and (2R)-2-{[(benzyloxy)carbonyl]amino}-3-(isoquinolin-6-yl)propanoic acid (474 mg, 1.35 mmol) were solubilised in DMF (10 ml), 4-methylmorpholine (370 μl, 3.4 mmol, CAS-RN: 109-02-4) and HATU (720 mg, 1.89 mmol) were added and the mixture was stirred under argon overnight at rt. The mixture was evaporated and purified by preparative HPLC (C18, acetonitrile/water with 0.1% formic acid) to give 456 mg (90% purity, 37% yield) of the target compound.

LC-MS (Method 1): Rₜ=1.25 min; MS (ESIpos): m/z=821 [M+H]⁺

Intermediate 256

### di-tert-butyl (2S)-2-({[(2S)-6-{[(2R)-2-amino-3-(isoquinolin-6-yl)propanoyl]amino}-1-tert-butoxy-1-oxohexan-2-yl]carbamoyl}amino)pentanedioate

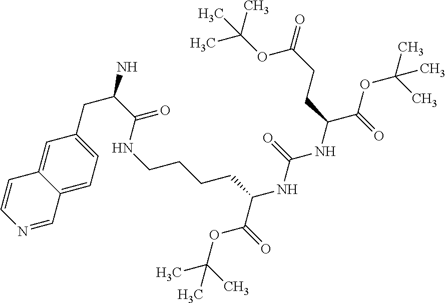 [SMILES: CC(C)(C)OC(=O)CC[C@H](NC(=O)N[C@@H](CCCCNC(=O)[C@H](N)Cc1ccc2cnccc2c1)C(=O)OC(C)(C)C)C(=O)OC(C)(C)C]

tri-tert-butyl (5R,12S,16S)-5-[(isoquinolin-6-yl)methyl]-3,6,14-trioxo-1-phenyl-2-oxa-4,7,13,15-tetraazaoctadecane-12,16,18-tricarboxylate (456 mg, 90% purity, 500 μmol) was solubilised in MeOH (4.1 ml), Palladium on carbon (53.3 mg, 10% purity, 50.0 μmol) was added and the mixture was purged with hydrogen. The mixture was stirred at rt under hydrogen atmosphere. The mixture was filtered over Celite, washed with MeOH and evaporated. The residue was purified by preparative HPLC (C18, acetonitrile/water with 0.1% formic acid) to give 130 mg (95% purity, 36% yield) of the target compound.

LC-MS (Method 1): Rₜ=0.94 min; MS (ESIpos): m/z=687 [M+H]⁺

¹H-NMR (400 MHz, DMSO-d6) δ[ppm]: 1.382 (16.00), 1.387 (7.54), 2.298 (0.73), 3.331 (0.66), 8.440 (0.50), 8.454 (0.45), 9.242 (0.56).

Intermediate 257

### tri-tert-butyl (3R,10S,14S)-1-{(1r,4S)-4-[({[(9H-fluoren-9-yl)methoxy]carbonyl}amino)methyl]cyclohexyl}-3-[(isoquinolin-6-yl)methyl]-1,4,12-trioxo-2,5,11,13-tetraazahexadecane-10,14,16-tricarboxylate

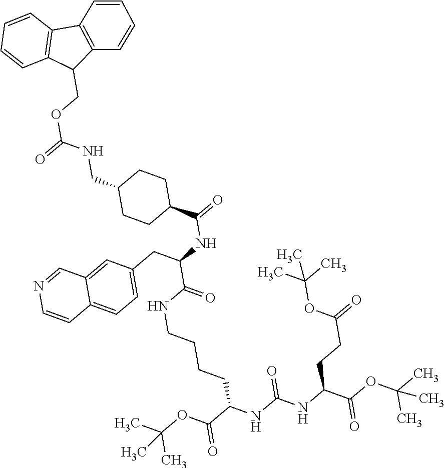 [SMILES: CC(C)(C)OC(=O)CC[C@H](NC(=O)N[C@@H](CCCCNC(=O)[C@@H](Cc1ccc2ccncc2c1)NC(=O)[C@H]1CC[C@H](CNC(=O)OCC2c3ccccc3-c3ccccc32)CC1)C(=O)OC(C)(C)C)C(=O)OC(C)(C)C]

(1r,4r)-4-[({[(9H-fluoren-9-yl)methoxy]carbonyl}amino)methyl]cyclohexane-1-carboxylic acid (79.1 mg, 208 μmol) was solubilised in DMF (2.9 ml), 4-methylmorpholine (63 μl, 570 μmol, CAS-RN: 109-02-4) and HATU (79.3 mg, 208 μmol) were added, stirred for 20 min at rt, di-tert-butyl (2S)-2-({[(2S)-6-{[(2R)-2-amino-3-(isoquinolin-6-yl)propanoyl]amino}-1-tert-butoxy-1-oxohexan-2-yl]carbamoyl}amino)pentanedioate (130 mg, 190 μmol) was added and the mixture was stirred under argon overnight at rt. The mixture was filtered and purified by preparative HPLC (C18, acetonitrile/water with 0.1% formic acid) to give 195 mg (98% yield) of the target compound.

LC-MS (Method 1): Rₜ=1.38 min; MS (ESIpos): m/z=1048 [M+H]⁺

Intermediate 258

### tri-tert-butyl (3R,10S,14S)-1-[(1r,4S)-4-(aminomethyl)cyclohexyl]-3-[(isoquinolin-6-yl)methyl]-1,4,12-trioxo-2,5,11,13-tetraazahexadecane-10,14,16-tricarboxylate

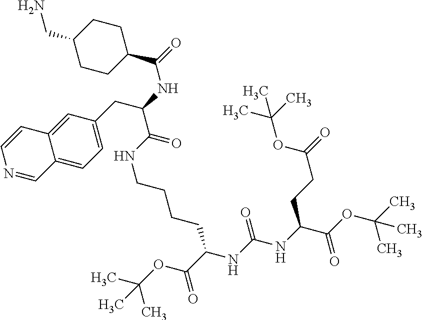 [SMILES: CC(C)(C)OC(=O)CC[C@H](NC(=O)N[C@@H](CCCCNC(=O)[C@@H](Cc1ccc2cnccc2c1)NC(=O)[C@H]1CC[C@H](CN)CC1)C(=O)OC(C)(C)C)C(=O)OC(C)(C)C]

tri-tert-butyl (3R,10S,14S)-1-{(1r,4S)-4-[({[(9H-fluoren-9-yl)methoxy]carbonyl}amino)methyl]cyclohexyl}-3-[(isoquinolin-6-yl)methyl]-1,4,12-trioxo-2,5,11,13-tetraazahexadecane-10,14,16-tricarboxylate (200 mg, 191 μmol) was solubilised in DMF (2.9 ml), piperidine (380 μl, 3.8 mmol) was added and the mixture was stirred under argon for 2 h at rt. The mixture was evaporated and purified by preparative HPLC (C18, acetonitrile/water with 0.1% formic acid) to give 43.0 mg (95% purity, 26% yield) of the target compound.

LC-MS (Method 1): Rₜ=1.01 min; MS (ESIpos): m/z=826 [M+H]⁺

¹H-NMR (400 MHz, DMSO-d6) δ[ppm]: 1.381 (13.30), 1.386 (16.00), 2.327 (0.47), 2.518 (1.96), 2.523 (1.22), 2.669 (0.49), 7.718 (0.45), 7.996 (0.44), 8.017 (0.50), 8.411 (0.81), 8.440 (0.55), 8.454 (0.50), 9.238 (0.64).

### Example 44-A

### (3R,10S,14S)-1-[(1r,4S)-4-(aminomethyl)cyclohexyl]-3-[(isoquinolin-6-yl)methyl]-1,4,12-trioxo-2,5,11,13-tetraazahexadecane-10,14,16-tricarboxylic acid

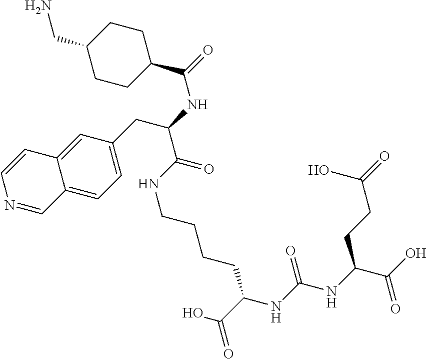 [SMILES: NC[C@H]1CC[C@H](C(=O)N[C@H](Cc2ccc3cnccc3c2)C(=O)NCCCC[C@H](NC(=O)N[C@@H](CCC(=O)O)C(=O)O)C(=O)O)CC1]

tri-tert-butyl (3R,10S,14S)-1-[(1r,4S)-4-(aminomethyl)cyclohexyl]-3-[(isoquinolin-6-yl)methyl]-1,4,12-trioxo-2,5,11,13-tetraazahexadecane-10,14,16-tricarboxylate (6.80 mg, 8.24 μmol) was solubilised in DCM (270 μl), TFA (130 μl, 1.6 mmol) was added and the mixture was stirred under argon overnight at rt. The mixture was evaporated and purified by preparative HPLC (C18, acetonitrile/water with 0.1% formic acid) to give 1.00 mg (90% purity, 17% yield) of the target compound.

LC-MS (Method 1): Rₜ=0.44 min; MS (ESIpos): m/z=657 [M+H]⁺

¹H-NMR (400 MHz, DMSO-d6) δ[ppm]: 0.834 (1.93), 0.862 (2.18), 1.026 (1.17), 1.053 (1.01), 1.144 (0.75), 1.168 (1.17), 1.234 (3.43), 1.258 (2.85), 1.275 (2.93), 1.432 (2.68), 1.454 (2.09), 1.605 (3.02), 1.640 (3.18), 1.767 (1.34), 1.808 (1.09), 1.987 (0.67), 2.020 (1.01), 2.050 (1.42), 2.074 (1.93), 2.141 (0.84), 2.162 (1.34), 2.176 (1.42), 2.229 (1.01), 2.251 (1.59), 2.271 (1.09), 2.284 (0.92), 2.518 (16.00), 2.523 (11.98), 2.539 (7.46), 2.573 (2.68), 2.945 (1.76), 2.978 (2.85), 3.004 (3.02), 3.089 (2.60), 3.104 (2.51), 3.157 (4.02), 3.169 (4.61), 3.950 (2.93), 4.533 (1.42), 4.544 (1.51), 6.140 (1.01), 6.442 (0.92), 7.561 (2.76), 7.582 (2.93), 7.696 (3.18), 7.711 (3.27), 7.747 (4.94), 7.959 (1.09), 7.992 (4.19), 8.013 (3.60), 8.233 (0.75), 8.280 (0.67), 8.437 (3.94), 8.452 (3.60), 9.234 (5.78).

Intermediate 259

### tri-tert-butyl (3R,10S,14S)-3-[(isoquinolin-6-yl)methyl]-1,4,12-trioxo-1-[(1r,4S)-4-({2-[4,7,10-tris(2-tert-butoxy-2-oxoethyl)-1,4,7,10-tetraazacyclododecan-1-yl]acetamido}methyl)cyclohexyl]-2,5,11,13-tetraazahexadecane-10,14,16-tricarboxylate

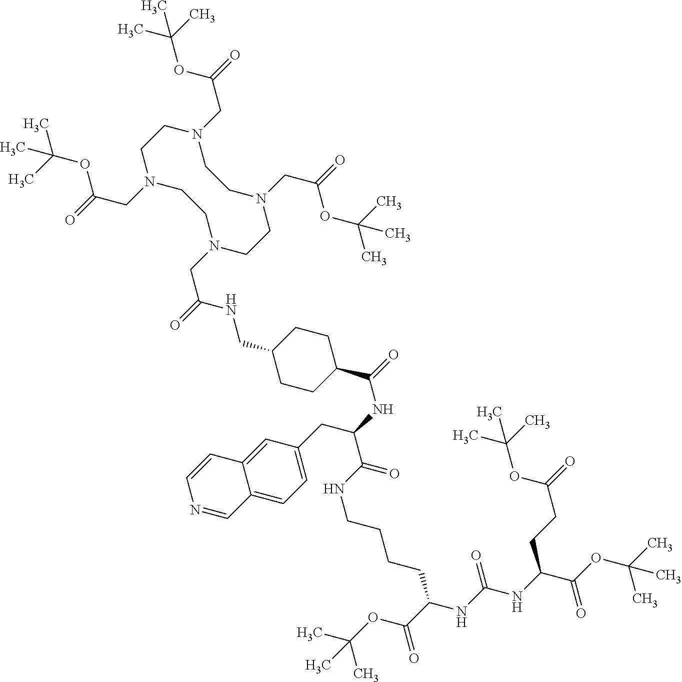 [SMILES: CC(C)(C)OC(=O)CC[C@H](NC(=O)N[C@@H](CCCCNC(=O)[C@@H](Cc1ccc2cnccc2c1)NC(=O)[C@H]1CC[C@H](CNC(=O)CN2CCN(CC(=O)OC(C)(C)C)CCN(CC(=O)OC(C)(C)C)CCN(CC(=O)OC(C)(C)C)CC2)CC1)C(=O)OC(C)(C)C)C(=O)OC(C)(C)C]

[4,7,10-tris(2-tert-butoxy-2-oxoethyl)-1,4,7,10-tetraazacyclododecan-1-yl]acetic acid (38.9 mg, 67.9 μmol; CAS-RN:[137076-54-1]), [(1H-benzotriazol-1-yl)oxy](dimethylamino)-N,N-dimethylmethaniminium hexafluoridophosphate(1−) (25.4 mg, 67.0 μmol; CAS-RN:94790-37-1) and N,N-diisopropylethylamine (8.7 μl, 51 μmol) were stirred in DMF (1.8 ml) for 10 min at rt. tri-tert-butyl (3R,10S,14S)-1-[(1r,4S)-4-(aminomethyl)cyclohexyl]-3-[(isoquinolin-6-yl)methyl]-1,4,12-trioxo-2,5,11,13-tetraazahexadecane-10,14,16-tricarboxylate (14.0 mg, 17.0 μmol) was added and the mixture was stirred overnight at rt. The mixture was evaporated and purified by preparative HPLC (C18, acetonitrile/water with 0.1% formic acid) to give 17.0 mg (30% purity, 22% yield) of the target compound.

LC-MS (Method 1): Rt=1.23 min; MS (ESIpos): m/z=1380 [M+H]⁺

### Example 44-B

### (3R,10S,14S)-3-[(isoquinolin-6-yl)methyl]-1,4,12-trioxo-1-[(1r,4S)-4-({2-[4,7,10-tris(carboxymethyl)-1,4,7,10-tetraazacyclododecan-1-yl]acetamido}methyl)cyclohexyl]-2,5,11,13-tetraazahexadecane-10,14,16-tricarboxylic acid

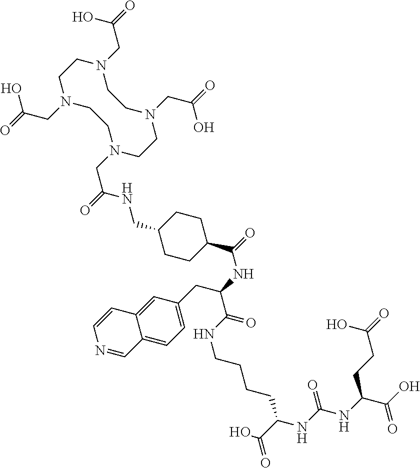 [SMILES: O=C(O)CC[C@H](NC(=O)N[C@@H](CCCCNC(=O)[C@@H](Cc1ccc2cnccc2c1)NC(=O)[C@H]1CC[C@H](CNC(=O)CN2CCN(CC(=O)O)CCN(CC(=O)O)CCN(CC(=O)O)CC2)CC1)C(=O)O)C(=O)O]

tri-tert-butyl (3R,10S,14S)-3-[(isoquinolin-6-yl)methyl]-1,4,12-trioxo-1-[(1r,4S)-4-({2-[4,7,10-tris(2-tert-butoxy-2-oxoethyl)-1,4,7,10-tetraazacyclododecan-1-yl]acetamido}methyl)cyclohexyl]-2,5,11,13-tetraazahexadecane-10,14,16-tricarboxylate (4.00 mg, 2.90 μmol) was solubilised in DCM (1.0 ml), TFA (510 μl) was added and the mixture was stirred under argon overnight at rt. The mixture was evaporated and purified by preparative HPLC (C18, acetonitrile/water with 0.1% formic acid) to give 2.50 mg (95% purity, 79% yield) of the target compound.

LC-MS (Method 1): Rₜ=0.46 min; MS (ESIneg): m/z=1042 [M−H]⁻

¹H-NMR (400 MHz, DMSO-d6) δ[ppm]: 0.784 (0.76), 0.814 (0.83), 1.228 (1.45), 1.319 (1.18), 1.352 (1.18), 1.463 (0.90), 1.594 (1.25), 1.722 (0.62), 1.738 (0.48), 1.907 (2.08), 2.032 (0.69), 2.209 (0.76), 2.224 (1.25), 2.246 (1.25), 2.263 (0.69), 2.318 (1.45), 2.322 (3.05), 2.326 (4.02), 2.331 (3.05), 2.336 (1.52), 2.518 (16.00), 2.522 (9.77), 2.659 (2.49), 2.664 (3.88), 2.669 (4.71), 2.673 (3.53), 2.678 (1.87), 2.729 (1.59), 2.889 (2.84), 2.979 (4.09), 3.095 (1.66), 3.152 (1.66), 3.185 (1.80), 3.436 (6.03), 3.987 (0.69), 4.000 (0.69), 4.073 (0.76), 4.086 (0.69), 4.534 (0.62), 6.298 (0.97), 6.317 (1.25), 6.333 (0.83), 7.555 (1.18), 7.576 (1.25), 7.699 (1.66), 7.714 (1.73), 7.731 (2.08), 7.993 (2.08), 8.015 (1.66), 8.075 (1.11), 8.136 (0.90), 8.437 (3.19), 8.451 (2.70), 9.233 (3.74).

### Example 44-B 227Th

### (3R,10S,14S)-3-[(isoquinolin-6-yl)methyl]-1,4,12-trioxo-1-{(1r,4S)-4-[(2-{4,7,10-tris[(carboxy-kappaO)methyl]-1,4,7,10-tetraazacyclododecan-1-yl-kappa²N¹,N⁴}acetamido)methyl]cyclohexyl}-2,5,11,13-tetraazahexadecane-10,14,16-tricarboxylato(3−)(²²⁷Th)Thorium

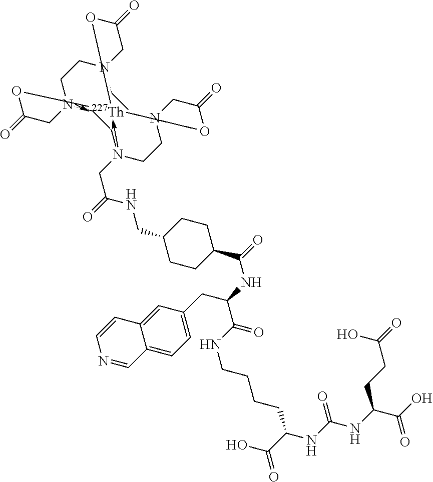

(3R,10S,14S)-3-[(isoquinolin-6-yl)methyl]-1,4,12-trioxo-1-[(1r,4S)-4-({2-[4,7,10-tris(carboxymethyl)-1,4,7,10-tetraazacyclododecan-1-yl]acetamido}methyl)cyclohexyl]-2,5,11,13-tetraazahexadecane-10,14,16-tricarboxylic acid was dissolved as 10 mM solution in DMSO and diluted to a 250 μM solution in 400 mM sodium acetate buffer (pH 5) containing 0.5 mg/mL pABA. 1.07 μl of this solution was used in a 40 μl labeling reaction. Thorium-227 in 400 mM sodium acetate buffer (pH 5) plus 20% EtOH was added giving a specific activity of 0.375 MBq/nmol and a RAC of 2.5 MBq/mL. The mixture was incubated at 95° C. for 60 min. The labelling efficiency was determined to be 97.8% by iTLC.

#45 Linker

Intermediate 260

### Methyl quinoline-7-carboxylate

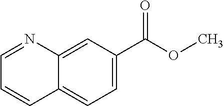

To a solution of 7-bromoquinoline (40.0 g, 192 mmol) in MeOH (400 ml) were added 1,1′-bis(diphenylphosphino)ferrocenepalladium(II) chloride (14.1 g, 19.2 mmol; CAS-RN:[72287-26-4]) and trimethylamine (54 ml, 380 mmol) at rt. The reaction mixture was stirred at 70° C. for 12 h under carbon monoxide (50 Psi). The reaction mixture was cooled to rt. The mixture was added to water. The mixture was extracted with EtOAc. The organic layer was evaporated under reduced pressure to give a residue. The residue was purified by flash chromatography (SiO2, petroleum ether/EtOAc gradient 0%-10%) to give 40.0 g of the target compound.

¹H NMR (400 MHz, DMSO-d₆) δ [ppm]=9.00 (dd, 1H), 8.56 (s, 1H), 8.43 (dd, 1H), 8.10-8.00 (m, 2H), 7.64 (dd, 1H), 3.92 (s, 3H).

Intermediate 261

### (quinolin-7-yl)MeOH

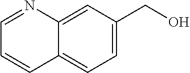 [SMILES: OCc1ccc2cccnc2c1]

To a solution of methyl quinoline-7-carboxylate (40.0 g, 214 mmol) in THF (400 ml) was added lithium aluminum hydride (8.11 g, 214 mmol; CAS-RN:[16853-85-3]) at 0° C. under nitrogen atmosphere. The reaction was stirred at rt for 2 h under nitrogen atmosphere. Saturated sodium potassium tartrate tetrahydrate was added to the reaction mixture. The mixture was stirred at rt for 12 h. The mixture was extracted with ethyl acetate. The organic layer was evaporated under reduced pressure. The residue was purified by flash chromatography (SiO2, petroleum ether/EtOAc gradient 5%-75%) to give 25.8 g (76% yield) of the target compound.

LC-MS (Method D): Rₜ=0.205 min; MS (ESIpos): m/z=160.0 [M+H]⁺.

Intermediate 262

### Quinoline-7-carbaldehyde

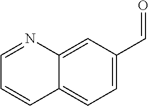 [SMILES: O=Cc1ccc2cccnc2c1]

To a solution of (quinolin-7-yl)MeOH (25.8 g, 162 mmol) in acetonitrile (250 ml) was added manganese(IV) oxide (70.4 g, 810 mmol) at rt. The reaction mixture was stirred at 80° C. for 12 h. The reaction mixture was cooled to rt and then filtered through celite. The organic layer was evaporated under reduced pressure to give 11.1 g (98% purity, 43% yield) of the target compound.

¹H NMR (400 MHz, DMSO-d₆) δ [ppm]=10.24 (s, 1H), 9.04 (dd, 1H), 8.61 (s, 1H), 8.46 (d, 1H), 8.12 (d, 1H), 7.97 (d, 1H), 7.68 (dd, 1H).

Intermediate 263

### Methyl (2Z)-2-{[(benzyloxy)carbonyl]amino}-3-(quinolin-7-yl)prop-2-enoate

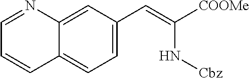 [SMILES: COC(=O)/C(=C/c1ccc2cccnc2c1)NC(=O)OCc1ccccc1]

1,8-Diazabicyclo(5.4.0)undec-7-ene (14 ml, 92 mmol) was added dropwise to a solution of methyl (2S)-{[(benzyloxy)carbonyl]amino}(dimethoxyphosphoryl)acetate (30.3 g, 91.6 mmol) in DCM (200 ml). After stirring at rt for 10 min, a solution of quinoline-7-carbaldehyde (12.0 g, 76.3 mmol) in DCM (100 ml) was added. The reaction was stirred at rt for 2 h. The reaction solvent was removed under reduced pressure. The residue was diluted with EtOAc. The solution was washed with 1M HCl and brine. The organic phase was dried. The filtrate was concentrated and purified by flash chromatography (SiO2, petroleum ether/EtOAc gradient 2%-33%) and trituration with methyl tert-butyl ether to give 12.5 g (45% yield) of the target compound.

LC-MS (Method D): Rₜ=0.639 min; MS (ESIpos): m/z=363.1 [M+H]⁺.

Intermediate 264

### Methyl (2R)-2-{[(benzyloxy)carbonyl]amino}-3-(quinolin-7-yl)propanoate

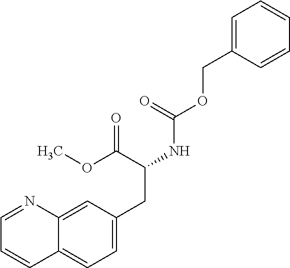 [SMILES: COC(=O)[C@@H](Cc1ccc2cccnc2c1)NC(=O)OCc1ccccc1]

To a solution of (E)-1-(((benzyloxy)carbonyl)amino)-2-(quinolin-7-yl)vinyl acetate (12.5 g, 34.5 mmol) in methanol (520 ml) was added (R)—[Rh(COD)(MaxPhos)]BF₄ (1.02 g, 1.72 mmol). The reaction mixture was stirred at room temperature for 96 hours under hydrogen atmosphere (50 psi). The reaction mixture was evaporated under reduced pressure to give a residue. The residue was dissolved in dichloromethane/methanol (10/1) and filtered through silica gel (200-300 mesh). The filtrate was evaporated under reduced pressure and purified by preparative-HPLC (Instrument: Shimadzu LC-20AP; Column: Phenomenex Synergi Max-RP 250*50 mm*10 μm; eluent A: 0.225% formic acid in water, eluent B: acetonitrile; gradient: 0-33 min 15-45% B; flow 100 ml/min; temperature: room temperature; Detector: UV 220/254 nm) to give methyl (2S)-2-{[(benzyloxy)carbonyl]amino}-3-(quinolin-7-yl)propanoate (4.80 g, 87% purity, 33% yield) as yellow oil.

LC-MS (Method C): Rₜ=0.786 min; MS (ESIpos): m/z=365.2 [M+H]⁺.

Intermediate 265

### (2R)-2-{[(benzyloxy)carbonyl]amino}-3-(quinolin-7-yl)propanoic acid

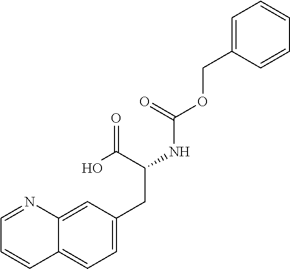 [SMILES: O=C(N[C@H](Cc1ccc2cccnc2c1)C(=O)O)OCc1ccccc1]

To a cooled solution (0° C.) of methyl (2R)-2-{[(benzyloxy)carbonyl]amino}-3-(quinolin-7-yl)propanoate (4.60 g, 12.6 mmol) in THF (70 ml) was added lithium hydroxide (15 ml, 2.0 M, 30 mmol; CAS-RN:[1310-65-2]). The reaction mixture was stirred at rt for 1 h. The reaction mixture was cooled to rt and evaporated under reduced pressure. The residue was dissolved in MeOH, the solution was adjusted to pH 5-6 through 1 M HCl and evaporated under reduced pressure. The residue was purified by preparative HPLC (C18, acetonitrile/water with 0.1% formic acid) to give 4.30 g (99% purity, 96% yield) of the target compound.

LC-MS (Method C): Rₜ=0.704 min; MS (ESIpos): m/z=351.0 [M+H]⁺.

¹H NMR (400 MHz, DMSO-d₆) δ=12.84 (br.s, 1H), 8.88 (dd, 1H), 8.33 (dd, 1H), 7.97-7.85 (m, 2H), 7.76 (d, 1H), 7.54 (dd, 1H), 7.51-7.48 (m, 1H), 7.32-7.09 (m, 5H), 4.98 (s, 2H), 4.40-4.28 (m, 1H), 3.32-3.28 (m, 1H), 3.07 (dd, 1H).

Intermediate 266

### tri-tert-butyl (5R,12S,16S)-3,6,14-trioxo-1-phenyl-5-[(quinolin-7-yl)methyl]-2-oxa-4,7,13,15-tetraazaoctadecane-12,16,18-tricarboxylate

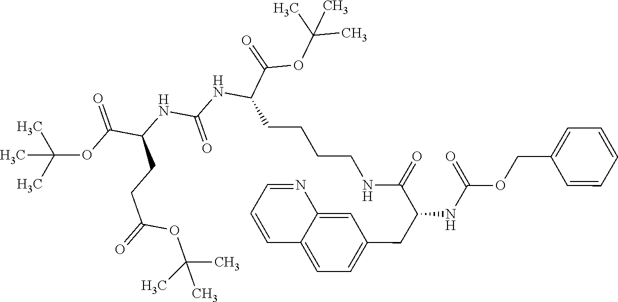 [SMILES: CC(C)(C)OC(=O)CC[C@H](NC(=O)N[C@@H](CCCCNC(=O)[C@@H](Cc1ccc2cccnc2c1)NC(=O)OCc1ccccc1)C(=O)OC(C)(C)C)C(=O)OC(C)(C)C]

di-tert-butyl N-{[(2S)-6-amino-1-tert-butoxy-1-oxohexan-2-yl]carbamoyl}-L-glutamate (974 mg, 2.00 mmol) and (2R)-2-{[(benzyloxy)carbonyl]amino}-3-(quinolin-7-yl)propanoic acid (700 mg, 2.00 mmol) were solubilised in DMF (15 ml), 4-methylmorpholine (550 μl, 5.0 mmol, CAS-RN: 109-02-4) and HATU (1.06 g, 2.80 mmol) were added and the mixture was stirred under argon overnight at rt. The mixture was evaporated and purified by preparative HPLC (C18, acetonitrile/water with 0.1% formic acid) to give 410 mg (98% purity, 25% yield) of the target compound.

LC-MS (Method 1): Rₜ=1.37 min; MS (ESIpos): m/z=821 [M+H]⁺

Intermediate 267

### di-tert-butyl (2S)-2-({[(2S)-6-{[(2R)-2-amino-3-(quinolin-7-yl)propanoyl]amino}-1-tert-butoxy-1-oxohexan-2-yl]carbamoyl}amino)pentanedioate

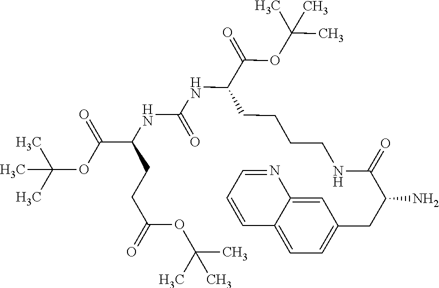 [SMILES: CC(C)(C)OC(=O)CC[C@H](NC(=O)N[C@@H](CCCCNC(=O)[C@H](N)Cc1ccc2cccnc2c1)C(=O)OC(C)(C)C)C(=O)OC(C)(C)C]

tri-tert-butyl (5R,12S,16S)-3,6,14-trioxo-1-phenyl-5-[(quinolin-7-yl)methyl]-2-oxa-4,7,13,15-tetraazaoctadecane-12,16,18-tricarboxylate (410 mg, 500 μmol) was solubilised in MeOH (4.1 ml), Palladium on carbon (53.2 mg, 10% purity, 50.0 μmol) was added and the mixture was purged with hydrogen. The mixture was stirred at rt under hydrogen atmosphere. The mixture was filtered over Celite, washed with MeOH and evaporated. The residue was purified by preparative HPLC (C18, acetonitrile/water with 0.1% formic acid) to give 151 mg (97% purity, 43% yield) of the target compound.

LC-MS (Method 1): Rₜ=1.01 min; MS (ESIpos): m/z=687 [M+H]⁺

Intermediate 268

### tri-tert-butyl (3R,10S,14S)-1-{(1r,4S)-4-[({[(9H-fluoren-9-yl)methoxy]carbonyl}amino)methyl]cyclohexyl}-1,4,12-trioxo-3-[(quinolin-7-yl)methyl]-2,5,11,13-tetraazahexadecane-10,14,16-tricarboxylate

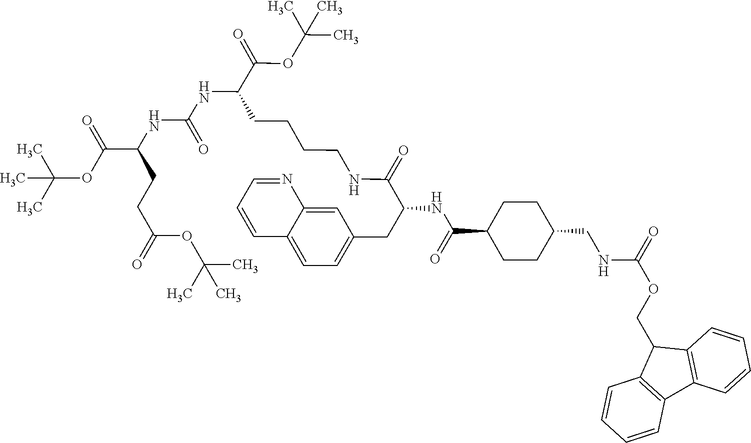 [SMILES: CC(C)(C)OC(=O)CC[C@H](NC(=O)N[C@@H](CCCCNC(=O)[C@@H](Cc1ccc2cccnc2c1)NC(=O)[C@H]1CC[C@H](CNC(=O)OCC2c3ccccc3-c3ccccc32)CC1)C(=O)OC(C)(C)C)C(=O)OC(C)(C)C]

(1r,4r)-4-[({[(9H-fluoren-9-yl)methoxy]carbonyl}amino)methyl]cyclohexane-1-carboxylic acid (91.9 mg, 242 μmol) was solubilised in DMF (3.4 ml), 4-methylmorpholine (73 μl, 660 μmol, CAS-RN: 109-02-4) and HATU (92.1 mg, 242 μmol) were added, stirred for 20 min at rt, di-tert-butyl (2S)-2-({[(2S)-6-{[(2R)-2-amino-3-(quinolin-7-yl)propanoyl]amino}-1-tert-butoxy-1-oxohexan-2-yl]carbamoyl}amino)pentanedioate (151 mg, 220 μmol) was added and the mixture was stirred under argon overnight at rt. The mixture was filtered and purified by preparative HPLC (C18, acetonitrile/water with 0.1% formic acid) to give 200 mg (97% purity, 84% yield) of the target compound.

LC-MS (Method 1): Rₜ=1.49 min; MS (ESIpos): m/z=1048 [M+H]⁺

Intermediate 269

### tri-tert-butyl (3R,10S,14S)-1-[(1r,4S)-4-(aminomethyl)cyclohexyl]-1,4,12-trioxo-3-[(quinolin-7-yl)methyl]-2,5,11,13-tetraazahexadecane-10,14,16-tricarboxylate

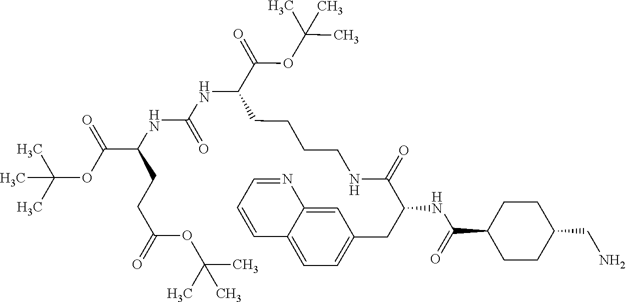 [SMILES: CC(C)(C)OC(=O)CC[C@H](NC(=O)N[C@@H](CCCCNC(=O)[C@@H](Cc1ccc2cccnc2c1)NC(=O)[C@H]1CC[C@H](CN)CC1)C(=O)OC(C)(C)C)C(=O)OC(C)(C)C]

tri-tert-butyl (3R,10S,14S)-1-{(1 r,4S)-4-[({[(9H-fluoren-9-yl)methoxy]carbonyl}amino)methyl]cyclohexyl}-1,4,12-trioxo-3-[(quinolin-7-yl)methyl]-2,5,11,13-tetraazahexadecane-10,14,16-tricarboxylate (200 mg, 191 μmol) was solubilised in DMF (2.9 ml), piperidine (380 μl, 3.8 mmol) was added and the mixture was stirred under argon for 2 h at rt. The mixture was evaporated and purified by preparative HPLC (C18, acetonitrile/water with 0.1% formic acid) to give 26.0 mg (96% purity, 16% yield) of the target compound.

LC-MS (Method 1): Rₜ=1.10 min; MS (ESIpos): m/z=826 [M+H]⁺

¹H-NMR (400 MHz, DMSO-d6) δ[ppm]: 1.211 (0.42), 1.230 (0.59), 1.364 (0.45), 1.382 (16.00), 1.387 (13.94), 1.390 (13.47), 2.518 (2.72), 2.523 (1.90), 7.473 (0.64), 7.846 (0.85), 7.865 (0.51), 8.387 (0.46), 8.839 (0.42), 8.843 (0.43), 8.850 (0.43).

### Example 45-A

### (3R,10S,14S)-1-[(1r,4S)-4-(aminomethyl)cyclohexyl]-1,4,12-trioxo-3-[(quinolin-7-yl)methyl]-2,5,11,13-tetraazahexadecane-10,14,16-tricarboxylic acid

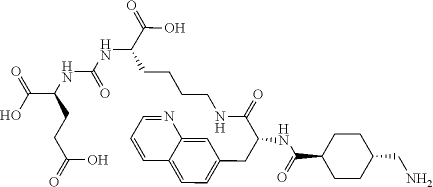 [SMILES: NC[C@H]1CC[C@H](C(=O)N[C@H](Cc2ccc3cccnc3c2)C(=O)NCCCC[C@H](NC(=O)N[C@@H](CCC(=O)O)C(=O)O)C(=O)O)CC1]

tri-tert-butyl (3R,10S,14S)-1-[(1r,4S)-4-(aminomethyl)cyclohexyl]-1,4,12-trioxo-3-[(quinolin-7-yl)methyl]-2,5,11,13-tetraazahexadecane-10,14,16-tricarboxylate (6.80 mg, 8.24 μmol) was solubilised in DCM (270 μl), TFA (130 μl, 1.6 mmol) was added and the mixture was stirred under argon overnight at rt. The mixture was evaporated and purified by preparative HPLC (C18, acetonitrile/water with 0.1% formic acid) to give 2.20 mg (95% purity, 39% yield) of the target compound.

LC-MS (Method 1): Rₜ=0.48 min; MS (ESIpos): m/z=657 [M+H]⁺

¹H-NMR (400 MHz, DMSO-d6) δ[ppm]: 0.835 (0.44), 0.852 (0.68), 0.868 (0.48), 1.233 (1.76), 1.353 (0.44), 1.452 (0.48), 1.606 (0.44), 2.518 (10.59), 2.523 (6.90), 2.540 (16.00), 2.556 (0.44), 2.568 (0.48), 3.166 (0.44), 3.200 (0.44), 3.929 (0.40), 7.444 (0.52), 7.454 (0.52), 7.464 (0.52), 7.475 (0.52), 7.487 (0.44), 7.512 (0.48), 7.847 (1.12), 7.867 (0.68), 8.286 (0.48), 8.303 (0.44), 8.841 (0.56), 8.845 (0.56), 8.851 (0.56), 8.856 (0.52).

Intermediate 270

### tri-tert-butyl (3R,10S,14S)-1,4,12-trioxo-3-[(quinolin-7-yl)methyl]-1-[(1r,4S)-4-({2-[4,7,10-tris(2-tert-butoxy-2-oxoethyl)-1,4,7,10-tetraazacyclododecan-1-yl]acetamido}methyl)cyclohexyl]-2,5,11,13-tetraazahexadecane-10,14,16-tricarboxylate

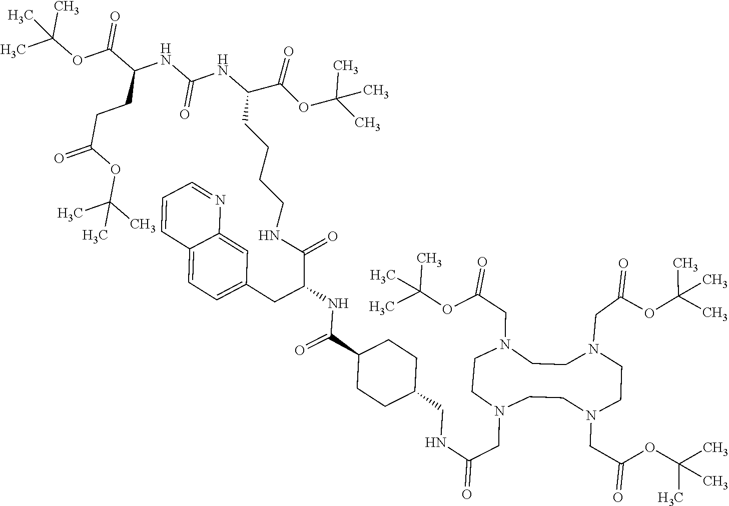 [SMILES: CC(C)(C)OC(=O)CC[C@H](NC(=O)N[C@@H](CCCCNC(=O)[C@@H](Cc1ccc2cccnc2c1)NC(=O)[C@H]1CC[C@H](CNC(=O)CN2CCN(CC(=O)OC(C)(C)C)CCN(CC(=O)OC(C)(C)C)CCN(CC(=O)OC(C)(C)C)CC2)CC1)C(=O)OC(C)(C)C)C(=O)OC(C)(C)C]

[4,7,10-tris(2-tert-butoxy-2-oxoethyl)-1,4,7,10-tetraazacyclododecan-1-yl]acetic acid (53.3 mg, 93.1 μmol; CAS-RN:[137076-54-1]), [(1H-benzotriazol-1-yl)oxy](dimethylamino)-N,N-dimethylmethaniminium hexafluoridophosphate(1−) (34.9 mg, 91.9 μmol; CAS-RN:94790-37-1) and N,N-diisopropylethylamine (12 μl, 70 μmol) were stirred in DMF (2.4 ml) for 10 min at rt. tri-tert-butyl (3R,10S,14S)-1-[(1r,4S)-4-(aminomethyl)cyclohexyl]-1,4,12-trioxo-3-[(quinolin-7-yl)methyl]-2,5,11,13-tetraazahexadecane-10,14,16-tricarboxylate (19.2 mg, 23.3 μmol) was added and the mixture was stirred overnight at rt. The mixture was filtered and purified by preparative HPLC (C18, acetonitrile/water with 0.1% formic acid) to give 26.0 mg (90% purity, 73% yield) of the target compound.

LC-MS (Method 1): Rₜ=1.28 min; MS (ESIpos): m/z=1381 [M+H]⁺

### Example 45-B

### (3R,10S,14S)-1,4,12-trioxo-3-[(quinolin-7-yl)methyl]-1-[(1r,4S)-4-({2-[4,7,10-tris(carboxymethyl)-1,4,7,10-tetraazacyclododecan-1-yl]acetamido}methyl)cyclohexyl]-2,5,11,13-tetraazahexadecane-10,14,16-tricarboxylic acid

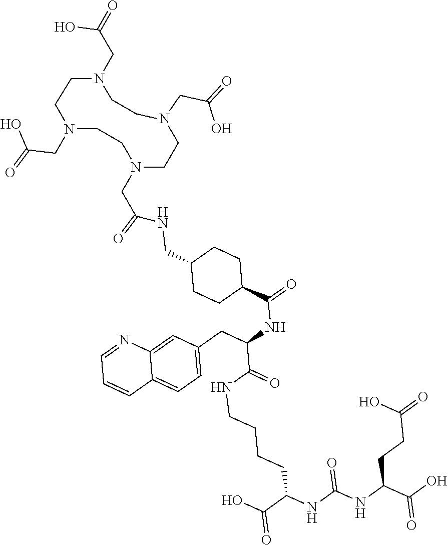 [SMILES: O=C(O)CC[C@H](NC(=O)N[C@@H](CCCCNC(=O)[C@@H](Cc1ccc2cccnc2c1)NC(=O)[C@H]1CC[C@H](CNC(=O)CN2CCN(CC(=O)O)CCN(CC(=O)O)CCN(CC(=O)O)CC2)CC1)C(=O)O)C(=O)O]

tri-tert-butyl (3R,10S,14S)-1,4,12-trioxo-3-[(quinolin-7-yl)methyl]-1-[(1r,4S)-4-({2-[4,7,10-tris(2-tert-butoxy-2-oxoethyl)-1,4,7,10-tetraazacyclododecan-1-yl]acetamido}methyl)cyclohexyl]-2,5,11,13-tetraazahexadecane-10,14,16-tricarboxylate (4.00 mg, 2.90 μmol) was solubilised in DCM (1.0 ml), TFA (510 μl) was added and the mixture was stirred under argon overnight at rt. The mixture was evaporated and purified by preparative HPLC (C18, acetonitrile/water with 0.1% formic acid) to give 1.00 mg (70% purity, 23% yield) of the target compound.

LC-MS (Method 1): Rₜ=0.48 min; MS (ESIpos): m/z=1042 [M−H]⁺

¹H-NMR (400 MHz, DMSO-d6) δ[ppm]: 0.815 (1.04), 0.851 (0.70), 0.999 (0.52), 1.015 (0.58), 1.044 (0.58), 1.074 (0.64), 1.230 (2.55), 1.306 (1.51), 1.321 (1.91), 1.339 (1.68), 1.439 (1.45), 1.456 (1.33), 1.603 (1.86), 1.640 (1.33), 1.687 (1.04), 1.703 (0.87), 1.724 (0.99), 1.739 (0.75), 1.876 (0.81), 1.894 (0.81), 2.004 (0.75), 2.033 (0.99), 2.073 (1.22), 2.211 (1.45), 2.232 (1.86), 2.240 (1.91), 2.261 (1.22), 2.322 (2.67), 2.326 (3.48), 2.331 (2.67), 2.336 (1.39), 2.518 (16.00), 2.522 (9.62), 2.539 (7.01), 2.644 (2.49), 2.659 (2.90), 2.664 (3.83), 2.669 (4.41), 2.673 (3.42), 2.892 (2.55), 2.958 (5.86), 2.991 (5.74), 3.013 (5.62), 3.086 (3.48), 3.150 (3.30), 3.173 (3.54), 3.185 (3.65), 3.433 (14.61), 3.957 (0.81), 3.977 (1.33), 3.990 (1.39), 4.011 (0.75), 4.050 (0.75), 4.070 (1.28), 4.084 (1.28), 4.104 (0.70), 4.532 (0.87), 6.307 (2.09), 6.327 (2.03), 7.445 (2.09), 7.455 (1.97), 7.466 (1.97), 7.476 (2.61), 7.504 (1.74), 7.849 (6.03), 7.869 (2.67), 7.978 (1.16), 8.073 (1.62), 8.140 (2.38), 8.287 (1.68), 8.304 (1.62), 8.427 (0.52), 8.441 (0.52), 8.845 (2.20), 8.849 (2.26), 8.855 (2.20), 8.860 (1.91), 9.201 (0.52).

### Example 45-B 227Th

### (3R,10S,14S)-1,4,12-trioxo-3-[(quinolin-7-yl)methyl]-1-{(1r,4S)-4-[(2-{4,7,10-tris[(carboxy-kappaO)methyl]-1,4,7,10-tetraazacyclododecan-1-yl-kappa²N¹,N⁴}acetamido)methyl]cyclohexyl}-2,5,11,13-tetraazahexadecane-10,14,16-tricarboxylato(3−)(²²⁷Th)Thorium

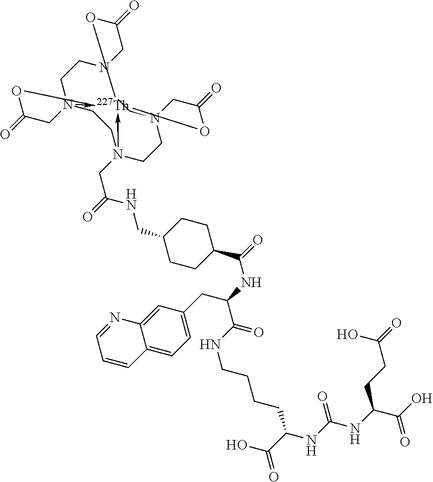

(3R,10S,14S)-1,4,12-trioxo-3-[(quinolin-7-yl)methyl]-1-[(1r,4S)-4-({2-[4,7,10-tris(carboxymethyl)-1,4,7,10-tetraazacyclododecan-1-yl]acetamido}methyl)cyclohexyl]-2,5,11,13-tetraazahexadecane-10,14,16-tricarboxylic acid was dissolved as 10 mM solution in DMSO and diluted to a 250 μM solution in 400 mM sodium acetate buffer (pH 5) containing 0.5 mg/mL pABA. 1.07 μl of this solution was used in a 40 μl labeling reaction. Thorium-227 in 400 mM sodium acetate buffer (pH 5) plus 20% EtOH was added giving a specific activity of 0.375 MBq/nmol and a RAC of 2.5 MBq/mL. The mixture was incubated at 95° C. for 60 min. The labelling efficiency was determined to be 89.9% by iTLC.

#46 Linker

Intermediate 271

### Trifluoroacetic acid-tri-tert-butyl (3S,10S,14S)-1-[(1r,4S)-4-(aminomethyl)cyclohexyl]-3-[(naphthalen-2-yl)methyl]-1,4,12-trioxo-2,5,11,13-tetraazahexadecane-10,14,16-tricarboxylate (1/1)

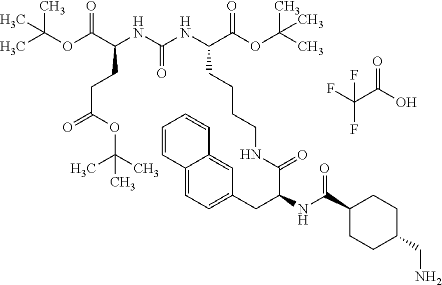 [SMILES: CC(C)(C)OC(=O)CC[C@H](NC(=O)N[C@@H](CCCCNC(=O)[C@H](Cc1ccc2ccccc2c1)NC(=O)[C@H]1CC[C@H](CN)CC1)C(=O)OC(C)(C)C)C(=O)OC(C)(C)C ; O=C(O)C(F)(F)F]

tri-tert-butyl (3S,10S,14S)-1-{(1r,4S)-4-[({[(9H-fluoren-9-yl)methoxy]carbonyl}amino)methyl]cyclohexyl}-3-[(naphthalen-2-yl)methyl]-1,4,12-trioxo-2,5,11,13-tetraazahexadecane-10,14,16-tricarboxylate (182 mg, 174 μmol) was stirred in piperidine (20%, in DMF) for 30 min at rt. The mixture was diluted with tert-butyl methyl ether/petrolether (9:1) and washed 2 times with sodium hydrogen carbonate solution. The organic layer was dried and evaporated. The residue was treated with acetonitrile, filtered and washed with acetonitrile. The filtrate was evaporated and purified by preparative HPLC (C18, acetonitrile/water with 0.1% formic acid) to give 141.0 mg (100% purity, 87% yield) of the target compound.

LC-MS (Method M): Rt=3.93 min; MS (ESIpos): m/z=825 [M+H]⁺

Intermediate 272

### N²-[4-(4-{3-(bis{2-[(3-hydroxy-1-methyl-2-oxo-1,2-dihydropyridine-4-carbonyl)amino]ethyl}amino)-2-[(bis{2-[(3-hydroxy-1-methyl-2-oxo-1,2-dihydropyridine-4-carbonyl)amino]ethyl}amino)methyl]propyl}anilino)-4-oxobutanoyl]-N⁵—[N-(2,2,4,6,7-pentamethyl-2,3-dihydro-1-benzofuran-5-sulfonyl)carbamimidoyl]-D-ornithyl-N⁵—[N-(2,2,4,6,7-pentamethyl-2,3-dihydro-1-benzofuran-5-sulfonyl)carbamimidoyl]-D-ornithine

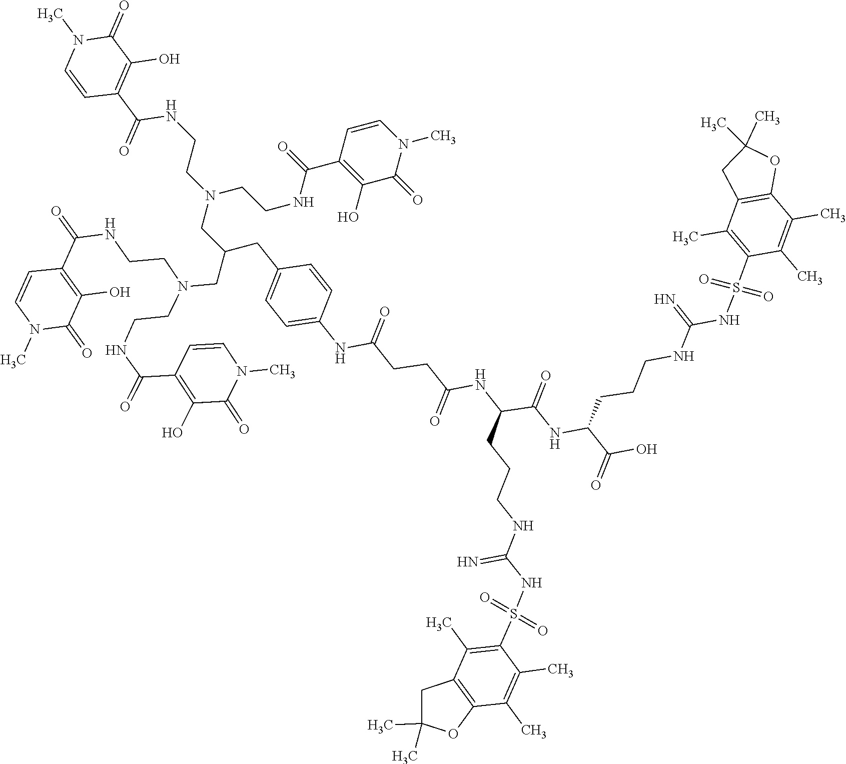 [SMILES: Cc1c(C)c(S(=O)(=O)NC(=N)NCCC[C@@H](NC(=O)[C@@H](CCCNC(=N)NS(=O)(=O)c2c(C)c(C)c3c(c2C)CC(C)(C)O3)NC(=O)CCC(=O)Nc2ccc(CC(CN(CCNC(=O)c3ccn(C)c(=O)c3O)CCNC(=O)c3ccn(C)c(=O)c3O)CN(CCNC(=O)c3ccn(C)c(=O)c3O)CCNC(=O)c3ccn(C)c(=O)c3O)cc2)C(=O)O)c(C)c2c1OC(C)(C)C2]

Fmoc-D-Arg(Pbf)-2-CT (CT=chlorotrityl resin) resin (0,378 mmol/g, 273.0 mg, 103 μmol) was mixed with piperidine 20% in DMF (6 mL) in a 10 mL filter syringe body and shaken for 30 minutes after which the supernatant solvent was removed by filtration and washed with DMF. The procedure was repeated one more time.

To the resulting H-D-Arg(Pbf)-2-CT was then added a solution of Fmoc-D-Arg(Pbf)-OH (134 mg, 206 μmol),) N,N′-diisopropylcarbodiimide (32 μl, 210 μmol) and ethyl cyanoglyoxylate-2-oxime (29.3 mg, 206 μmol; CAS-RN [3849-21-6]) in DMF (4 mL), then shaken for 2 hours. Afterwards the supernatant solution was removed and the resin washed with several time with DMF. The coupling procedure was repeated 1×.

The resin was then mixed with piperidine 20% in DMF (6 mL) and then shaken for 30 minutes for the deprotection step; then the supernatant solvent was subsequently removed and the resin was washed with DMF. The deprotection procedure was repeated 1×.

The resulting H-D-Arg(Pbf)-D-Arg(Pbf)-2-CT was mixed with 3 mL of DCM, to which then was added PYAOP (80.7 mg, 155 μmol in 1 ml DMF; CAS-RN:[156311-83-0]), 4-methylmorpholine (34 μl, 310 μmol) and then 4-(4-{3-(bis{2-[(3-hydroxy-1-methyl-2-oxo-1,2-dihydropyridine-4-carbonyl)amino]ethyl}amino)-2-[(bis{2-[(3-hydroxy-1-methyl-2-oxo-1,2-dihydropyridine-4-carbonyl)amino]ethyl}amino)methyl]propyl}anilino)-4-oxobutanoic acid (109 mg, 103 μmol, solid) under an argon counter-stream. The mixture was shaken for 2 h, the supernatant solution was removed, and then the resin washed with DCM. The coupling procedure was repeated 2×, and completion of reaction was monitored by test cleavage/analytical HPLC. Cleavage of the target compound was facilitated by adding 1,1,1,3,3,3-hexafluoro-2-propanol (20% in DCM, 6 mL), shaking the mixture for 30 minutes at rt and then removing the supernatant solution by filtration. The procedure was repeated twice. The combined filtrates were then concentrated, the residue taken up in a mixture of acetonitrile and water (8 mL+2 mL), and the solution was lyophilized to afford 103 mg (88% purity, 46% yield) of the target compound.

LC-MS (Method N): Rₜ=10.53 min; MS (ESI pos): m/z=937 [M+2H]2⁺

Intermediate 273

### tri-tert-butyl (3S,10S,14S)-1-{(1R,4S)-4-[({(2R)-2-({(2R)-2-[4-(4-{3-(bis{2-[(3-hydroxy-1-methyl-2-oxo-1,2-dihydropyridine-4-carbonyl)amino]ethyl}amino)-2-[(bis{2-[(3-hydroxy-1-methyl-2-oxo-1,2-dihydropyridine-4-carbonyl)amino]ethyl}amino)methyl]propyl}anilino)-4-oxobutanamido]-5-[N′-(2,2,4,6,7-pentamethyl-2,3-dihydro-1-benzofuran-5-sulfonyl)carbamimidamido]pentanoyl}amino)-5-[N′-(2,2,4,6,7-pentamethyl-2,3-dihydro-1-benzofuran-5-sulfonyl)carbamimidamido]pentanoyl}amino)methyl]cyclohexyl}-3-[(naphthalen-2-yl)methyl]-1,4,12-trioxo-2,5,11,13-tetraazahexadecane-10,14,16-tricarboxylate

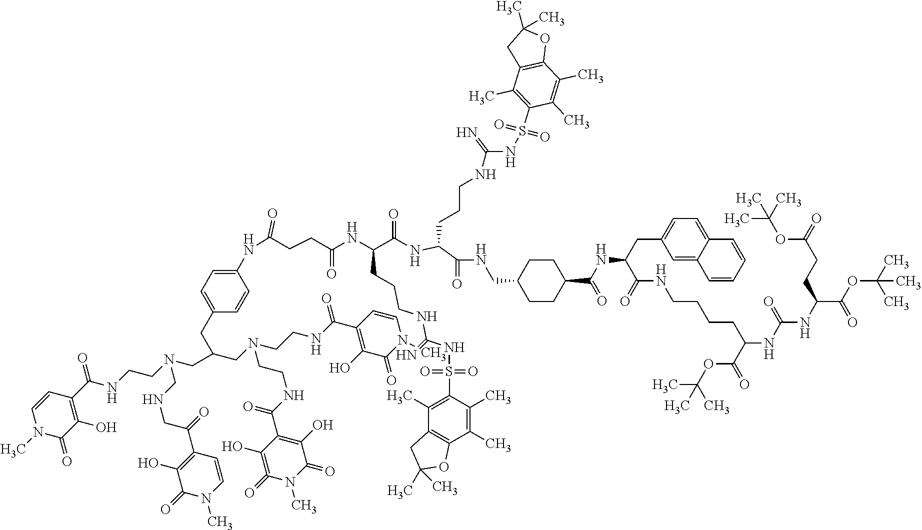

N²-[4-(4-{3-(bis{2-[(3-hydroxy-1-methyl-2-oxo-1,2-dihydropyridine-4-carbonyl)amino]ethyl}amino)-2-[(bis{2-[(3-hydroxy-1-methyl-2-oxo-1,2-dihydropyridine-4-carbonyl)amino]ethyl}amino)methyl]propyl}anilino)-4-oxobutanoyl]-N⁵—[N-(2,2,4,6,7-pentamethyl-2,3-dihydro-1-benzofuran-5-sulfonyl)carbamimidoyl]-D-ornithyl-N⁵—[N-(2,2,4,6,7-pentamethyl-2,3-dihydro-1-benzofuran-5-sulfonyl)carbamimidoyl]-D-ornithine (101 mg, 53.9 pmol), trifluoroacetic acid-tri-tert-butyl (3S,10S,14S)-1-[(1r,4S)-4-(aminomethyl)cyclohexyl]-3-[(naphthalen-2-yl)methyl]-1,4,12-trioxo-2,5,11,13-tetraazahexadecane-10,14,16-tricarboxylate (1/1) (75.9 mg, 80.9 μmol) and PYAOP (56.2 mg, 107.9 μmol; CAS-RN:[156311-83-0]) were solubilised in DCM (6.0 μl) and DMF (0.50 μl), 4-methylmorpholine (21 μl, 187 μmol; CAS-RN:[109-02-4]) was added and the mixture was stirred for at rt and overnight at 7° C. (fridge). TFA (21 μl, 270 μmol) in acetonitrile (3.0 ml) was added and the DCM was evaporated. The residue was purified by preparative HPLC (C18, acetonitrile/water with 0.1% formic acid) to give 77.0 mg (68% purity, 36.5% yield) of the target compound.

LC-MS (Method N): Rₜ=13.67 min; MS (ESI pos): m/z=1339.65 [M+2H]2⁺

### Example 46-E

### (3S,10S,14S)-1-[(1R,4S)-4-({[(2R)-2-({(2R)-2-[4-(4-{3-(bis{2-[(3-hydroxy-1-methyl-2-oxo-1,2-dihydropyridine-4-carbonyl)amino]ethyl}amino)-2-[(bis{2-[(3-hydroxy-1-methyl-2-oxo-1,2-dihydropyridine-4-carbonyl)amino]ethyl}amino)methyl]propyl}anilino)-4-oxobutanamido]-5-carbamimidamidopentanoyl}amino)-5-carbamimidamidopentanoyl]amino}methyl)cyclohexyl]-3-[(naphthalen-2-yl)methyl]-1,4,12-trioxo-2,5,11,13-tetraazahexadecane-10,14,16-tricarboxylic acid

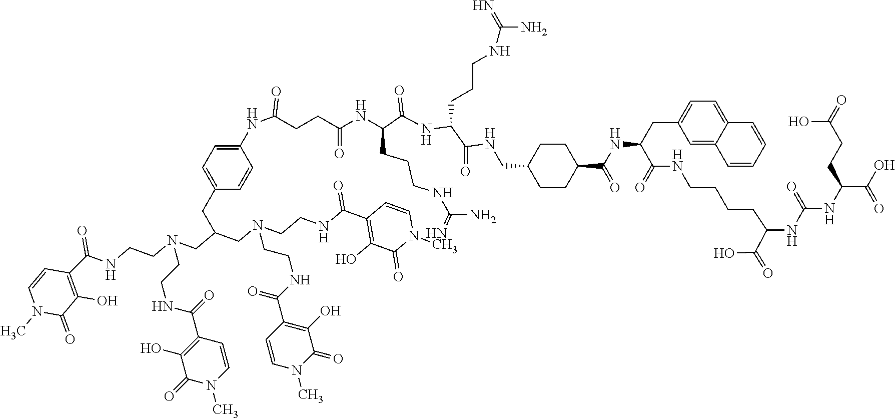 [SMILES: Cn1ccc(C(=O)NCCN(CCNC(=O)c2ccn(C)c(=O)c2O)CC(Cc2ccc(NC(=O)CCC(=O)N[C@H](CCCNC(=N)N)C(=O)N[C@H](CCCNC(=N)N)C(=O)NC[C@H]3CC[C@H](C(=O)N[C@@H](Cc4ccc5ccccc5c4)C(=O)NCCCCC(NC(=O)N[C@@H](CCC(=O)O)C(=O)O)C(=O)O)CC3)cc2)CN(CCNC(=O)c2ccn(C)c(=O)c2O)CCNC(=O)c2ccn(C)c(=O)c2O)c(O)c1=O]

tri-tert-butyl (3S,10S,14S)-1-{(1R,4S)-4-[({(2R)-2-({(2R)-2-[4-(4-{3-(bis{2-[(3-hydroxy-1-methyl-2-oxo-1,2-dihydropyridine-4-carbonyl)amino]ethyl}amino)-2-[(bis{2-[(3-hydroxy-1-methyl-2-oxo-1,2-dihydropyridine-4-carbonyl)amino]ethyl}amino)methyl]propyl}anilino)-4-oxobutanamido]-5-[N′-(2,2,4,6,7-pentamethyl-2,3-dihydro-1-benzofuran-5-sulfonyl)carbamimidamido]pentanoyl}amino)-5-[N′-(2,2,4,6,7-pentamethyl-2,3-dihydro-1-benzofuran-5-sulfonyl)carbamimidamido]pentanoyl}amino)methyl]cyclohexyl}-3-[(naphthalen-2-yl)methyl]-1,4,12-trioxo-2,5,11,13-tetraazahexadecane-10,14,16-tricarboxylate (101 mg, 37.7 μmol) was solubilised in DCM (1.5 mL), a TFA solution (1.3 mL in 1.5 mL DCM) was added under argon and the reaction mixture was stirred for 40 min in a sealed vessel. The mixture was evaporated at 30° C. and purified by preparative HPLC (C18, acetonitrile/water with 0.1% TFA) to give 28.0 mg (95% purity, 68% yield) of the target compound.

LC-MS (Method N): Rₜ=7.89 min; MS (ESIpos): m/z=1003 [M+2H]2⁺

### Example 46-E ²²⁷Th

### (3S,10S,14S)-1-[(1R,4S)-4-({[(2R)-2-{[(2R)-2-{4-[4-(3−[bis(2-{[3-(hydroxy-kappaO)-1-methyl-2-oxo-1,2-dihydropyridine-4-carbonyl]amino}ethyl)amino]-2-{[bis(2-{[3-(hydroxy-kappaO)-1-methyl-2-oxo-1,2-dihydropyridine-4-carbonyl]amino}ethyl)amino]methyl}propyl)anilino]-4-oxobutanamido}-5-carbamimidamidopentanoyl]amino}-5-carbamimidamidopentanoyl]amino}methyl)cyclohexyl]-3-[(naphthalen-2-yl)methyl]-1,4,12-trioxo-2,5,11,13-tetraazahexadecane-10,14,16-tricarboxylato(4−)(²²⁷Th)Thorium

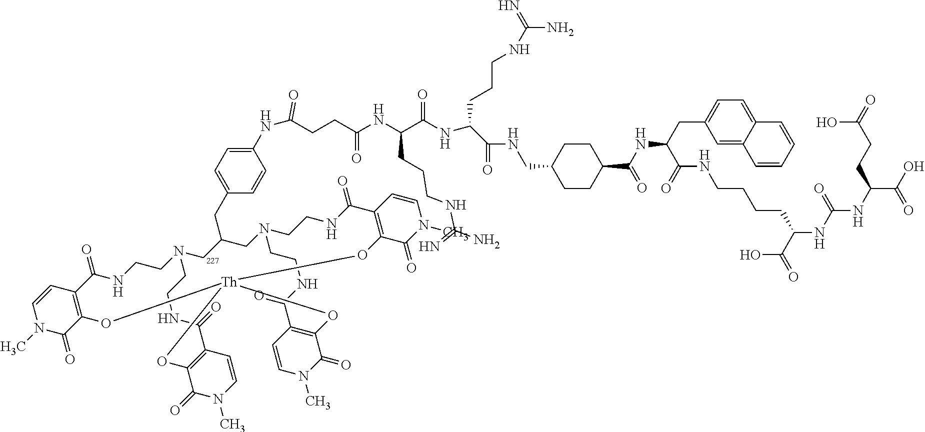

(3S,10S,14S)-1-[(1R,4S)-4-({[(2R)-2-({(2R)-2-[4-(4-{3-(bis{2-[(3-hydroxy-1-methyl-2-oxo-1,2-dihydropyridine-4-carbonyl)amino]ethyl}amino)-2-[(bis{2-[(3-hydroxy-1-methyl-2-oxo-1,2-dihydropyridine-4-carbonyl)amino]ethyl}amino)methyl]propyl}anilino)-4-oxobutanamido]-5-carbamimidamidopentanoyl}amino)-5-carbamimidamidopentanoyl]amino}methyl)cyclohexyl]-3-[(naphthalen-2-yl)methyl]-1,4,12-trioxo-2,5,11,13-tetraazahexadecane-10,14,16-tricarboxylic acid was dissolved in 30 mM citrate buffer (pH 5.5) containing 50 mg/mL sucrose, 2 mM EDTA and 0.5 mg/mL pABA was added thorium-227 dissolved in 30 mM citrate buffer (pH 5.5) resulting in a specific activity of 0.3 MBq/nmol and a RAC of 4.6 MBq/mL. The mixture was adjusted to pH 8 by adding 1M carbonate buffer. The mixture was incubated at room temperature for 60 min. The labelling efficiency was determined to be 98.9% by iTLC.

#47 Linker

Intermediate 274

### di-tert-butyl N-[(4-nitrophenoxy)carbonyl]-L-glutamate

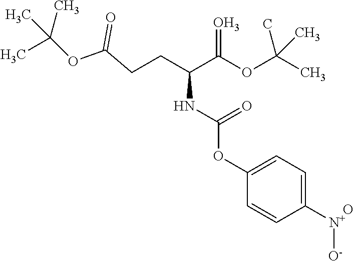

A mixture of di-tert-butyl L-glutamate-hydrogen chloride (2.00 g, 6.76 mmol), 4-nitrophenyl carbonochloridate (1.36 g, 6.76 mmol), N,N-diisopropylethylamine (2.5 ml, 14 mmol), and THF (55 mL) was stirred at 30° C. for 3 h. After that the mixture was cooled to r.t., separated between ethyl acetate and 1N aqueous sodium hydroxide solution. The organic phase was washed 3× with 1N aqueous sodium hydroxide solution and once with brine and filtered through a phase separator. After concentration under reduced pressure the crude product was directly used in the next step.

Intermediate 275

### (2S)-2-({[(2S)-1,5-di-tert-butoxy-1,5-dioxopentan-2-yl]carbamoyl}amino)-6-({[(9H-fluoren-9-yl)methoxy]carbonyl}amino)hexanoic acid

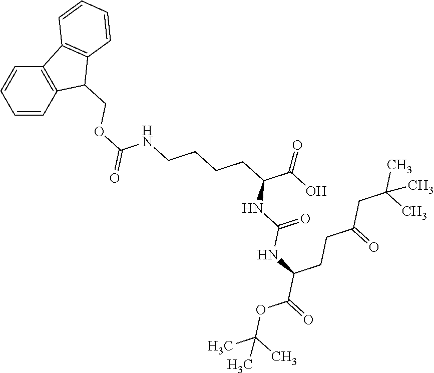 [SMILES: CC(C)(C)CC(=O)CC[C@H](NC(=O)N[C@@H](CCCCNC(=O)OCC1c2ccccc2-c2ccccc21)C(=O)O)C(=O)OC(C)(C)C]

To a solution of N⁶-{[(9H-fluoren-9-yl)methoxy]carbonyl}-L-lysine (2.49 g, 6.76 mmol) in DCM (10 mL) was added at r.t. N,N-diisopropylethylamine (1.3 ml, 7.4 mmol) and di-tert-butyl N-[(4-nitrophenoxy)carbonyl]-L-glutamate (2.87 g, 6.76 mmol). The mixture stirred for 14 h at r.t. After that the mixture was filtered and the residue washed with DCM. The filtrate was concentrated under reduced pressure and the crude product purified by column chromatography (SiO₂, DCM/EtOH gradient) and thereafter by recrystallization from ethyl acetate/hexane. The solids were filtered off, washed with ethyl acetate, and dried under to give the title compound (1.72 g, 92% purity, 36% yield).

LC-MS (Method 1): Rₜ=1.42 min; MS (ESIpos): m/z=1308 [M+H]⁺

1H-NMR (400 MHz, DMSO-d6) delta [ppm]: 1.154 (0.42), 1.172 (0.84), 1.190 (0.41), 1.263 (0.21), 1.281 (0.20), 1.368 (0.57), 1.388 (16.00), 1.987 (1.49), 2.198 (0.16), 2.204 (0.20), 2.209 (0.18), 2.227 (0.19), 2.518 (0.31), 2.523 (0.20), 2.728 (0.28), 2.888 (0.32), 2.944 (0.28), 2.959 (0.27), 4.017 (0.44), 4.026 (0.25), 4.035 (0.56), 4.047 (0.25), 4.053 (0.23), 4.199 (0.21), 4.215 (0.18), 4.273 (0.61), 4.290 (0.38), 6.252 (0.27), 6.272 (0.26), 6.303 (0.24), 6.324 (0.23), 7.280 (0.24), 7.305 (0.33), 7.308 (0.34), 7.324 (0.80), 7.327 (0.77), 7.343 (0.52), 7.345 (0.51), 7.390 (0.39), 7.409 (0.62), 7.428 (0.27), 7.672 (0.55), 7.691 (0.49), 7.877 (0.65), 7.895 (0.59).

Intermediate 276

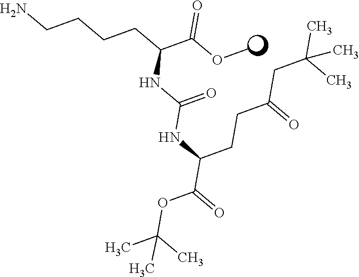 [SMILES: COC(=O)[C@H](CCCCN)NC(=O)N[C@@H](CCC(=O)CC(C)(C)C)C(=O)OC(C)(C)C]

2-Chlorotrityl chloride (CTC) resin (1.58 mmol/g, 98.0 mg, 150 μmol) were mixed with DCM (2 mL) and filled into a 2 mL syringe body and shaken for 1 h after which the supernatant solvent was removed. After that (2S)-2-({[(2S)-1,5-di-tert-butoxy-1,5-dioxopentan-2-yl]carbamoyl}amino)-6-{[(9H-fluoren-9-ylmethoxy)carbonyl]amino}hexanoic acid (198 mg, 303 μmol) and 4-methyl morpholine (100 μl, 910 μmol) were dissolved in DCM (2 mL) and added to the resin. The mixture was shaken for 14 h at r.t. After that the supernatant was removed and 4-methylmorpholine (6.7 μl, 61 μmol) dissolved in methanol (0.25 mL) was added to the resin. After 15 min the supernatant was removed and the resin was washed with DCM (5×2 mL), DMF (5×2 mL), DMF/methanol (1:1, 5×2 mL), methanol (3×2 mL), and DMF (5×2 mL). Then a mixture of piperidine in DMF (1:4, 2 mL) was added to the resin twice and the mixture was shaken for 5 min each. After that the supernatant was removed and the resin was washed with DMF (3×2 mL) and DCM (3×2 mL).

Intermediate 277

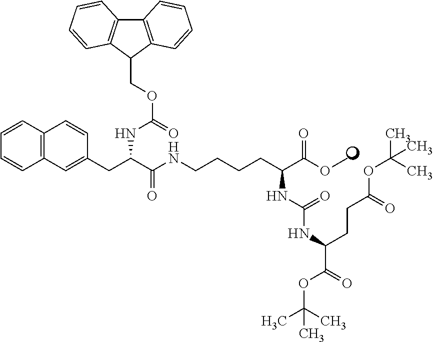 [SMILES: COC(=O)[C@H](CCCCNC(=O)[C@H](Cc1ccc2ccccc2c1)NC(=O)OCC1c2ccccc2-c2ccccc21)NC(=O)N[C@@H](CCC(=O)OC(C)(C)C)C(=O)OC(C)(C)C]

The resin-bound starting material from the previous step was mixed with a solution of (2S)-2-({[(9H-fluoren-9-yl)methoxy]carbonyl}amino)-3-(naphthalen-2-yl)propanoic acid (262 mg, 600 μmol), HATU (222 mg, 585 μmol), and 4-methylmorpholine (130 μl, 1.2 mmol) in DMF (1.5 mL) and the mixture was shaken at r.t. for 1 h. After that the resin was washed with DMF (5×2 mL) and DCM (5×2 mL).

Intermediate 278

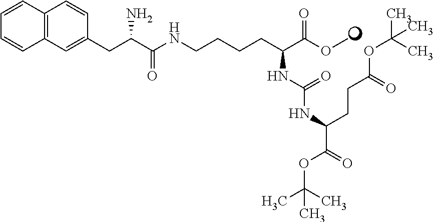 [SMILES: COC(=O)[C@H](CCCCNC(=O)[C@@H](N)Cc1ccc2ccccc2c1)NC(=O)N[C@@H](CCC(=O)OC(C)(C)C)C(=O)OC(C)(C)C]

The resin-bound starting material from the previous step was washed with DMF (3×2 mL) and then a mixture of DMF/piperidine (4:1, 2×2 mL) was added and the mixture was shaken at r.t. for 15 min each. Then the resin was washed with DMF (5×2 mL) and DCM (3×2 mL).

Intermediate 279

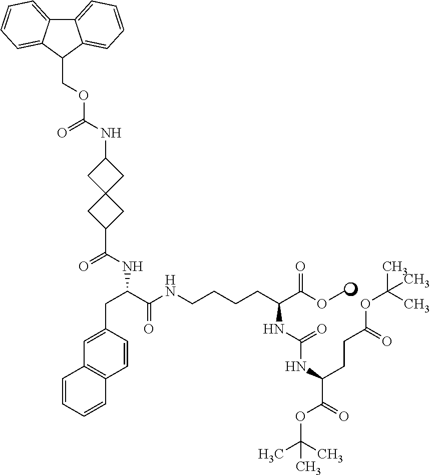 [SMILES: COC(=O)[C@H](CCCCNC(=O)[C@H](Cc1ccc2ccccc2c1)NC(=O)C1CC2(CC(NC(=O)OCC3c4ccccc4-c4ccccc43)C2)C1)NC(=O)N[C@@H](CCC(=O)OC(C)(C)C)C(=O)OC(C)(C)C]

The resin-bound starting material from the previous step was mixed with a solution of 6-({[(9H-fluoren-9-yl)methoxy]carbonyl}amino)spiro[3.3]heptane-2-carboxylic acid (110 mg, 293 μmol), HATU (111 mg, 293 μmol), and 4-methylmorpholine (66 μl, 600 μmol) in DMF (1.1 mL) and the mixture was shaken at r.t. for 1 h. After that the resin was washed with DMF (5×2 mL) and DCM (5×2 mL).

Intermediate 280

### (2S)-6-{[(2S)-2-[(6-aminospiro[3.3]heptane-2-carbonyl)amino]-3-(naphthalen-2-yl)propanoyl]amino}-2-({[(2S)-1,5-di-tert-butoxy-1,5-dioxopentan-2-yl]carbamoyl}amino)hexanoic acid

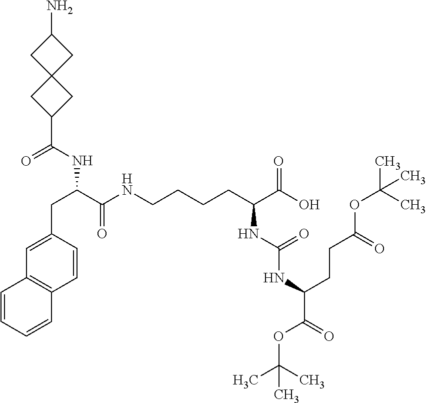 [SMILES: CC(C)(C)OC(=O)CC[C@H](NC(=O)N[C@@H](CCCCNC(=O)[C@H](Cc1ccc2ccccc2c1)NC(=O)C1CC2(CC(N)C2)C1)C(=O)O)C(=O)OC(C)(C)C]

The resin-bound starting material from the previous step was washed with DMF (3×2 mL) and then a mixture of DMF/piperidine (4:1, 2×2 mL) was added and the mixture was shaken at r.t. for 15 min each. Then the resin was washed with DMF (5×2 mL) and DCM (3×2 mL). After that the product was released from the resin by adding a mixture of HFIP/TIPS/water (95:2.5:2.5) (3×2 mL, 15 min each). The combined solutions were concentrated under reduced pressure and the crude product was purified by preparative HPLC (C18, acetonitrile/1% formic acid) to give the title compound (10 mg, 85% purity).

LC-MS (Method 1): Rₜ=1.07 min; MS (ESIpos): m/z=766 [M+H]⁺.

### Example 47-A

### (3S,10S,14S)-1-(6-aminospiro[3.3]heptan-2-yl)-3-[(naphthalen-2-yl)methyl]-1,4,12-trioxo-2,5,11,13-tetraazahexadecane-10,14,16-tricarboxylic acid

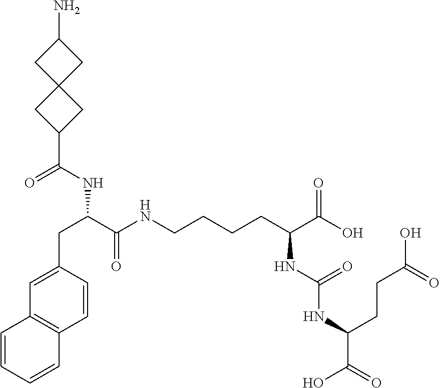 [SMILES: NC1CC2(C1)CC(C(=O)N[C@@H](Cc1ccc3ccccc3c1)C(=O)NCCCC[C@H](NC(=O)N[C@@H](CCC(=O)O)C(=O)O)C(=O)O)C2]

A mixture of (2S)-6-{[(2S)-2-[(6-aminospiro[3.3]heptane-2-carbonyl)amino]-3-(naphthalen-2-yl)propanoyl]amino}-2-({[(2S)-1,5-di-tert-butoxy-1,5-dioxopentan-2-yl]carbamoyl}amino)hexanoic acid (10.0 mg, 13.1 μmol) and hydrogenchloride (4M in dioxane, 33 μL) was stirred for 14 h at r.t. After that the mixture was concentrated under reduced pressure to give the title compound (8.0 mg, 95% purity, 89% yield).

LC-MS (Method 1): Rₜ=0.73 min; MS (ESIpos): m/z=654 [M+H]⁺.

¹H-NMR (400 MHz, DMSO-d6) δ[ppm]: 0.851 (0.40), 1.232 (2.88), 1.295 (1.36), 1.313 (1.36), 1.352 (0.64), 1.460 (0.72), 1.481 (0.56), 1.579 (0.72), 1.600 (0.56), 1.682 (0.48), 1.696 (0.64), 1.717 (0.80), 1.731 (0.56), 1.815 (0.80), 1.848 (1.12), 1.870 (1.36), 1.900 (1.36), 1.919 (1.36), 1.939 (1.12), 2.004 (1.60), 2.026 (2.00), 2.050 (2.32), 2.073 (1.60), 2.093 (0.64), 2.211 (1.04), 2.227 (1.68), 2.249 (1.76), 2.268 (1.20), 2.289 (0.88), 2.331 (3.52), 2.518 (16.00), 2.522 (10.72), 2.539 (1.68), 2.673 (3.44), 2.859 (0.96), 2.879 (1.28), 2.900 (1.44), 2.922 (0.88), 2.970 (0.72), 2.987 (0.72), 3.015 (0.72), 3.031 (0.80), 3.047 (0.64), 3.064 (0.96), 3.100 (0.56), 3.426 (0.40), 3.483 (0.40), 3.983 (0.48), 4.004 (0.88), 4.018 (0.96), 4.038 (0.48), 4.067 (0.56), 4.089 (1.12), 4.101 (1.12), 4.122 (0.48), 4.514 (0.80), 4.526 (0.80), 6.276 (1.44), 6.297 (1.52), 6.312 (1.68), 6.332 (1.52), 7.373 (1.44), 7.395 (1.60), 7.426 (0.64), 7.440 (1.52), 7.443 (1.44), 7.457 (2.72), 7.462 (1.68), 7.472 (1.36), 7.488 (0.56), 7.668 (2.72), 7.778 (3.20), 7.797 (3.20), 7.840 (1.84), 7.859 (1.60), 7.901 (0.96), 7.912 (0.96), 7.923 (0.96), 7.933 (0.80), 7.993 (1.12), 8.133 (0.40).

#48 Linker

Intermediate 290

### Methyl (2E)-3-(quinolin-6-yl)prop-2-enoate

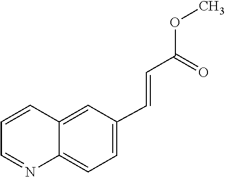 [SMILES: COC(=O)/C=C/c1ccc2ncccc2c1]

To a solution of 6-bromoquinoline (20.0 g, 96.1 mmol) and methyl prop-2-enoate (24.8 g, 288 mmol) in N,N-dimethylformamide (200 ml) were added dichlorobis(triphenylphosphine) palladium(II) (3.37 g, 4.81 mmol) and potassium carbonate (33.2 g, 240 mmol) at room temperature. The reaction mixture was heated to 100° C. and stirred at 100° C. for 12 hours. The reaction mixture was poured to water (1000 ml) and the mixture was extracted with ethyl acetate. The organic layer was evaporated under reduced pressure to give a residue. The residue was purified by chromatography (200-300 mesh, petroleum ether:ethyl acetate=10:1, then 5:1, then 2:1) to give methyl 3-(quinolin-6-yl)acrylate (11.1 g, 54% yield) as yellow oil.

¹H NMR (400 MHz, DMSO-d₆) δ [ppm]=8.91 (dd, J=4.4, 2.0 Hz, 1H), 8.37-8.31 (m, 1H), 8.24 (d, J=1.6 Hz, 1H), 8.11 (dd, J=8.8, 1.6 Hz, 1H), 8.00 (d, J=8.8 Hz, 1H), 7.81 (d, J=16.0 Hz, 1H), 7.55 (dd, J=8.0, 4.0 Hz, 1H), 6.78 (d, J=16.0 Hz, 1H), 3.74 (s, 3H).

Intermediate 291

### Methyl 3-(1,2,3,4-tetrahydroquinolin-6-yl)propanoate

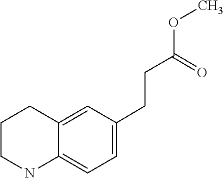 [SMILES: COC(=O)CCc1ccc2c(c1)CCCN2]

To a solution of methyl (2E)-3-(quinolin-6-yl)prop-2-enoate (11.1 g, 52.1 mmol) in methanol (100 ml) was added palladium on carbon (2.77 g, 10% purity, 2.60 mmol) at room temperature. The reaction mixture was stirred at room temperature for 12 hours under hydrogen atmosphere. The reaction mixture was filtered through celite. The filtrate was evaporated under reduced pressure to give a residue. The residue was purified by chromatography (200-300 mesh, petroleum ether:ethyl acetate=10:1, then 5:1, then 2:1) to give methyl 3-(1,2,3,4-tetrahydroquinolin-6-yl)propanoate (10.1 g, 88% yield)) as a yellow oil.

Intermediate 292

### Methyl 3-(quinolin-6-yl)propanoate

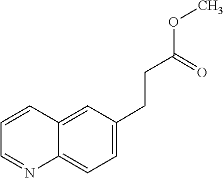 [SMILES: COC(=O)CCc1ccc2ncccc2c1]

To a solution of methyl 3-(1,2,3,4-tetrahydroquinolin-6-yl)propanoate (10.1 g, 46.1 mmol) in a mixed solvent of 1,4-dioxane (100 ml) and water (5.0 ml) was added sodium periodate (19.7 g, 92.1 mmol) at room temperature under nitrogen atmosphere. The reaction mixture was heated to 80° C. and stirred at 80° C. for 48 hours. The reaction mixture was cooled to room temperature. Saturated sodium thiosulfate and ethyl acetate were added to the reaction mixture. After separated, the organic layer was evaporated under reduced pressure to give a residue. The residue was purified by chromatography (200-300 mesh, petroleum ether:ethyl acetate=9:1, then 4:1, then 2:1) to give methyl 3-(quinolin-6-yl)propanoate (5.20 g, 52% yield) as brown oil.

¹H NMR (400 MHz, DMSO-d₆) δ [ppm]=8.84 (dd, J=1.6, 4.0 Hz, 1H), 8.27 (d, J=8.0 Hz, 1H), 7.94 (d, J=8.8 Hz, 1H), 7.77 (d, J=0.8 Hz, 1H), 7.65 (dd, J=8.4, 2.0 Hz, 1H), 7.49 (dd, J=8.4, 4.4 Hz, 1H), 3.58 (s, 3H), 3.05 (t, J=7.6 Hz, 2H), 2.75 (t, J=7.6 Hz, 2H).

Intermediate 293

### 3-(quinolin-6-yl)propanoic acid

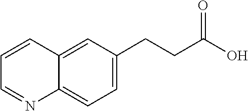 [SMILES: O=C(O)CCc1ccc2ncccc2c1]

To a solution of methyl 3-(quinolin-6-yl)propanoate (5.20 g, 24.2 mmol) in a mixed solvent of tetrahydrofuran (10 ml) and water (2.0 ml) was added lithium hydroxide (1.16 g, 48.3 mmol) at room temperature. The reaction mixture was stirred at room temperature for 12 hours. The reaction mixture was adjusted to pH=3-4 through hydrochloric acid (1M). The mixture was extracted with ethyl acetate. The organic layer was evaporated under reduced pressure to give 3-(quinolin-6-yl)propanoic acid (3.60 g, 74% yield) as a yellow solid.

LC-MS (Method D): Rₜ=0.486 min; MS (ESIpos): m/z=202.0 [M+H]⁺.

Intermediate 294

### 3-(quinolin-6-yl)propanoyl chloride

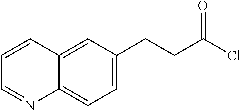 [SMILES: O=C(Cl)CCc1ccc2ncccc2c1]

To a solution of 3-(quinolin-6-yl)propanoic acid (3.60 g, 17.9 mmol) in dichloromethane (50 ml) was added oxalyl chloride (3.1 ml, 36 mmol) at room temperature under nitrogen atmosphere. After stirring at room temperature for 2 hours under nitrogen atmosphere, the reaction mixture was evaporated under reduced pressure to give 3-(quinolin-6-yl)propanoyl chloride (3.93 g, 100% yield) as a brown solid.

Intermediate 295

### (4S)-4-benzyl-3-[3-(quinolin-6-yl)propanoyl]-1,3-oxazolidin-2-one

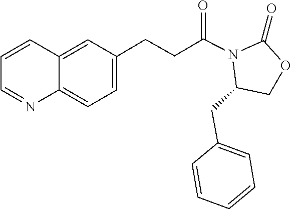 [SMILES: O=C(CCc1ccc2ncccc2c1)N1C(=O)OC[C@@H]1Cc1ccccc1]

To a stirred solution of (4S)-4-benzyl-1,3-oxazolidin-2-one (3.17 g, 17.9 mmol) in tetrahydrofuran (90 ml) was added n-butyllithium (2.5 M in hexane, 11 ml, 27 mmol) under an atmosphere of nitrogen at −78° C. After stirring at −78° C. for 30 min, 3-(quinolin-6-yl)propanoyl chloride (3.93 g, 17.9 mmol) was added to the reaction mixture. The reaction mixture was stirred at room temperature for 12 hours. The reaction mixture was quenched with saturated ammonium chloride and extracted with ethyl acetate. The organic layer was evaporated under reduced pressure to give a residue. The residue was purified by chromatography (200-300 mesh, petroleum ether:ethyl acetate=20:1, then 10:1, then 4:1, then 2:1) to give (4S)-4-benzyl-3-[3-(quinolin-6-yl)propanoyl]-1,3-oxazolidin-2-one (2.90 g, 45% yield) as yellow oil.

¹H NMR (400 MHz, DMSO-d₆) δ [ppm]=8.85 (dd, J=4.0, 1.6 Hz, 1H), 8.31 (dd, J=7.6, 0.8 Hz, 1H), 7.98 (d, J=8.8 Hz, 1H), 7.86-7.78 (m, 1H), 7.71 (dd, J=8.4, 2.0 Hz, 1H), 7.50 (dd, J=8.0, 4.4 Hz, 1H), 7.26-7.20 (m, 3H), 7.16-7.08 (m, 2H), 4.71-4.62 (m, 1H), 4.35-4.15 (m, 2H), 3.42-3.27 (m, 2H), 3.19-3.08 (m, 2H), 3.02-2.86 (m, 2H).

Intermediate 296

### (4S)-3-[(2S)-2-azido-3-(quinolin-6-yl)propanoyl]-4-benzyl-1,3-oxazolidin-2-one

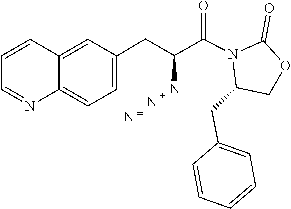

A solution of (4S)-4-benzyl-3-[3-(quinolin-6-yl)propanoyl]-1,3-oxazolidin-2-one (1.90 g, 5.27 mmol) in tetrahydrofuran (10 ml) was added to a solution of lithium bis(trimethylsilyl)amide (5.8 ml, 1.0 M, 5.8 mmol) in tetrahydrofuran (20 ml) at −70° C. After stirring at −70° C. for 30 minutes, a solution of 2,4,6-tri(propan-2-yl)benzene-1-sulfonyl azide (1.96 g, 6.33 mmol) in tetrahydrofuran (10 ml) was added at the same temperature. The reaction mixture was stirred at −70° C. for 2 minutes. Then acetic acid was added to the reaction mixture at −70° C. The reaction mixture was warmed to room temperature and stirred at room temperature for 12 hours. Saturated ammonium chloride was added to the reaction mixture. The solution was extracted with ethyl acetate. The organic layer was evaporated under reduced pressure to give a residue. The residue was purified by preparative HPLC (Instrument: Shimadzu LC-20AP; Column: Phenomenex Synergi Max-RP 250*50 mm*10 μm; eluent A: 0.225% formic acid in water, eluent B: acetonitrile; gradient: 0-35 min 25-55% B; flow 100 ml/min; temperature: room temperature; Detector: UV 220/254 nm) to give (4S)-3-[(2S)-2-azido-3-(quinolin-6-yl)propanoyl]-4-benzyl-1,3-oxazolidin-2-one (1.10 g, 52% yield) as a light yellow solid.

LC-MS (Method C): Rₜ=0.695 min; MS (ESIpos): m/z=401.9 [M+H]⁺.

¹H NMR (400 MHz, DMSO-d₆) δ [ppm]=8.98 (dd, J=4.4, 1.6 Hz, 1H), 8.55 (d, J=7.6 Hz, 1H), 8.08 (d, J=8.4 Hz, 1H), 7.98 (d, J=1.2 Hz, 1H), 7.82 (dd, J=8.8, 2.0 Hz, 1H), 7.66 (dd, J=8.4, 4.8 Hz, 1H), 7.36-7.21 (m, 5H), 5.35-5.27 (m, 1H), 4.77-4.69 (m, 1H), 4.39 (t, J=8.8 Hz, 1H), 4.29 (dd, J=8.8, 2.8 Hz, 1H), 3.51-3.45 (m, 1H), 3.18-3.06 (m, 2H), 3.05-2.97 (m, 1H).

Intermediate 297

### (2S)-2-azido-3-(quinolin-6-yl)propanoic acid

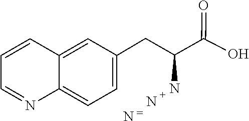

To a solution of (4S)-3-[(2S)-2-azido-3-(quinolin-6-yl)propanoyl]-4-benzyl-1,3-oxazolidin-2-one (900 mg, 2.24 mmol) in a mixed solvent of tetrahydrofuran (9.0 ml) and water (2 ml) was added hydrogen peroxide (1.5 ml, 30% purity, 15 mmol) dropwise at 0° C. After stirring at 0° C. for 10 minutes, a solution of lithium hydroxide (118 mg, 4.93 mmol) in water (1 ml) was added dropwise to the reaction mixture at 0° C. The reaction mixture was stirred at 0° C. for 2 hours. Saturated sodium sulfite and water was added to the reaction mixture. The mixture was washed with ethyl acetate. The aqueous layer was adjusted to pH=4-5 with hydrochloric acid (1M) and extracted with ethyl acetate. The organic layer was evaporated under reduced pressure to give (2S)-2-azido-3-(quinolin-6-yl)propanoic acid (430 mg, 79% yield) as light yellow oil.

LC-MS (Method D): Rₜ=0.526 min; MS (ESIpos): m/z=243.0 [M+H]⁺.

Intermediate 298

### (2S)-2-amino-3-(quinolin-6-yl)propanoic acid

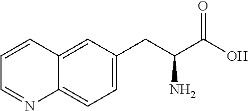 [SMILES: N[C@@H](Cc1ccc2ncccc2c1)C(=O)O]

To a solution of (2S)-2-azido-3-(quinolin-6-yl)propanoic acid (400 mg, 1.65 mmol) in a mixed solvent of tetrahydrofuran (2.0 ml) and hydrochloric acid (2.0 ml, 1M) was added triphenylphosphine (455 mg, 1.73 mmol) at room temperature. After stirring at room temperature for 12 hours, the reaction mixture was used to next step without further purification.

LC-MS (Method C): Rₜ=0.122 min; MS (ESIpos): m/z=216.9 [M+H]⁺.

Intermediate 299

### (2S)-2-amino-3-(quinolin-6-yl)propanoic acid

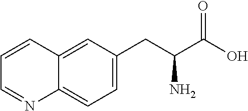 [SMILES: N[C@@H](Cc1ccc2ncccc2c1)C(=O)O]

To a solution of (2S)-2-azido-3-(quinolin-6-yl)propanoic acid (5.20 g, 89% purity, 19.1 mmol) in a mixed solvent of tetrahydrofuran (20 ml) and hydrochloric acid (20 ml, 1M in water) was added triphenylphosphine (5.51 g, 21.0 mmol) at room temperature. After stirring at room temperature for 12 hours, the reaction mixture was washed with ethyl acetate. The aqueous layer was lyophilized to give (2S)-2-amino-3-(quinolin-6-yl)propanoic acid (5.10 g, 80% purity, 99% yield) as a yellow solid.

LC-MS (Method A): Rₜ=0.137 min; MS (ESIpos): m/z=216.9 [M+H]⁺.

Intermediate 300

### (2S)-2-{[(benzyloxy)carbonyl]amino}-3-(quinolin-6-yl)propanoic acid

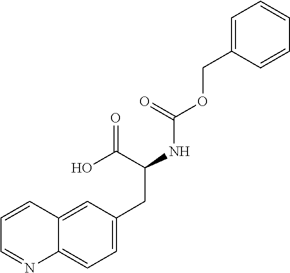 [SMILES: O=C(N[C@@H](Cc1ccc2ncccc2c1)C(=O)O)OCc1ccccc1]

To a solution of (2S)-2-amino-3-(quinolin-6-yl)propanoic acid (350 mg, 1.62 mmol) in tetrahydrofuran (6.8 ml) were added sodium hydrogen carbonate (272 mg, 3.24 mmol) and benzyl chloroformate (230 μl, 1.6 mmol) at 14° C. The reaction mixture was stirred at 14° C. for 16 hours. The reaction mixture was warmed to 28° C. and stirred at 28° C. for 16 hours. Saturated sodium hydrogen carbonate was added to the reaction mixture. The mixture was washed with ethyl acetate. The aqueous layer was adjusted to pH=4-5 by hydrochloric acid (1M) and extracted with ethyl acetate. The organic layer was evaporated under reduced pressure to give a crude product. The crude product was purified by preparative HPLC (Instrument: Gilson-281; Column: Phenomenex Synergi C18 150*25 mm*10 μm; eluent A: 0.225% formic acid in water, eluent B: acetonitrile; gradient: 0-10 min 7-37% B; flow 25 ml/min; temperature: room temperature; Detector: UV 220/254 nm) to give the product (82% LCMS purity). The product was purified again by preparative HPLC (Instrument: Gilson-281; Column: Phenomenex Synergi C18 150*25 mm*10 μm; eluent A: 0.225% formic acid in water, eluent B: acetonitrile; gradient: 0-10 min 3-33% B; flow 25 ml/min; temperature: room temperature; Detector: UV 220/254 nm) to give (2S)-2-{[(benzyloxy)carbonyl]amino}-3-(quinolin-6-yl)propanoic acid (4.00 mg, 98% purity, 1% yield) as a white solid.

LC-MS (Method C): Rₜ=0.532 min; MS (ESIpos): m/z=350.9 [M+H]⁺.

¹H NMR (400 MHz, DMSO-d₆) δ [ppm]=8.85 (dd, J=4.0, 1.6 Hz, 1H), 8.25 (d, J=8.4 Hz, 1H), 7.92 (d, J=8.4 Hz, 1H), 7.79 (s, 1H), 7.67 (dd, J=8.4, 1.6 Hz, 1H), 7.59 (d, J=7.6 Hz, 1H), 7.50 (dd, J=8.4, 4.4 Hz, 1H), 7.28-7.08 (m, 5H), 4.94 (s, 2H), 4.34-4.24 (m, 1H), 3.27-3.25 (m, 1H), 3.08-3.02 (m, 1H).

Intermediate 301

### (2S)-2-{[(benzyloxy)carbonyl]amino}-3-(quinolin-6-yl)propanoic acid

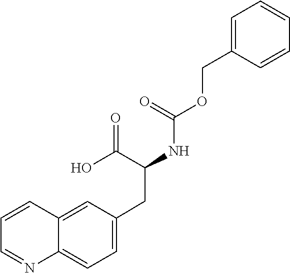 [SMILES: O=C(N[C@@H](Cc1ccc2ncccc2c1)C(=O)O)OCc1ccccc1]

To a solution of (2S)-2-amino-3-(quinolin-6-yl)propanoic acid (1.90 g, 80% purity, 7.03 mmol) in a mixed solvent of tetrahydrofuran (10 ml) and water (10 ml) were added sodium hydrogen carbonate (1.77 g, 21.1 mmol) and N-(benzyloxycarbonyloxy)succinimide (1.75 g, 7.03 mmol) at room temperature. The reaction mixture was stirred at room temperature for 16 hours. The reaction mixture was evaporated under reduced pressure. The resulting suspension was filtered. The filter cake was washed with water and methanol to give (2S)-2-{[(benzyloxy)carbonyl]amino}-3-(quinolin-6-yl)propanoic acid (1.44 g, 98% purity, 99% ee %, 57% yield) as a white solid. The filtrate was purified by preparative HPLC (Instrument: Shimadzu LC-20AP; Column: Phenomenex Synergi Max-RP 250*80 mm*10 μm; eluent A: 0.225% formic acid in water, eluent B: acetonitrile; gradient: 0-20 min 20-48% B; flow 160 ml/min; temperature: room temperature; Detector: UV 220/254 nm) to give (2S)-2-{[(benzyloxy)carbonyl]amino}-3-(quinolin-6-yl)propanoic acid (660 mg, 96% purity, 82% ee %, 25% yield) as a light yellow solid.

LC-MS (Method A): Rₜ=0.645 min; MS (ESIpos): m/z=351.0 [M+H]⁺.

¹H NMR (400 MHz, DMSO-d₆) δ [ppm]=12.82 (br. s, 1H), 8.86 (dd, J=4.0, 1.6 Hz, 1H), 8.26 (d, J=7.6 Hz, 1H), 7.93 (d, J=8.8 Hz, 1H), 7.81 (s, 1H), 7.77-7.64 (m, 2H), 7.51 (dd, J=8.4, 4.0 Hz, 1H), 7.29-7.15 (m, 5H), 4.94 (s, 2H), 4.39-4.29 (m, 1H), 3.28-3.25 (m, 1H), 3.11-3.00 (m, 1H).

Intermediate 302

### tri-tert-butyl (5S,12S,16S)-3,6,14-trioxo-1-phenyl-5-[(quinolin-6-yl)methyl]-2-oxa-4,7,13,15-tetraazaoctadecane-12,16,18-tricarboxylate

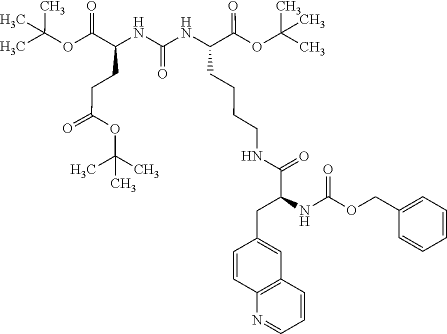 [SMILES: CC(C)(C)OC(=O)CC[C@H](NC(=O)N[C@@H](CCCCNC(=O)[C@H](Cc1ccc2ncccc2c1)NC(=O)OCc1ccccc1)C(=O)OC(C)(C)C)C(=O)OC(C)(C)C]

A mixture of di-tert-butyl N-{[(2S)-6-amino-1-tert-butoxy-1-oxohexan-2-yl]carbamoyl}-L-glutamate (1.95 g, 4.00 mmol), (2S)-2-{[(benzyloxy)carbonyl]amino}-3-(quinolin-6-yl)propanoic acid (1.40 g, 4.00 mmol), 4-methylmorpholine (1.3 ml, 12 mmol), HATU (2.28 g, 5.99 mmol), and DMF (31 ml, 400 mmol) was stirred for 3 h at r.t. After that the mixture was separated between DCM and water. The organic phase was filtered through a phase separator and concentrated under reduced pressure. The crude product was redissolved in DCM, filtered, and the filtrate concentrated under reduced pressure and purified by preparative HPLC (C18, acetonitrile/1% formic acid) to give the title compound (1.28 g, 1.25 mmol, 31% yield).

Intermediate 303

### di-tert-butyl (2S)-2-({[(2S)-6-{[(2S)-2-amino-3-(quinolin-6-yl)propanoyl]amino}-1-tert-butoxy-1-oxohexan-2-yl]carbamoyl}amino)pentanedioate

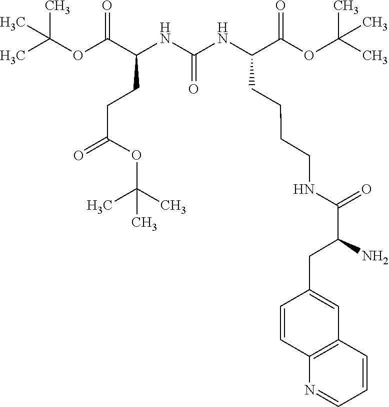 [SMILES: CC(C)(C)OC(=O)CC[C@H](NC(=O)N[C@@H](CCCCNC(=O)[C@@H](N)Cc1ccc2ncccc2c1)C(=O)OC(C)(C)C)C(=O)OC(C)(C)C]

To a solution of tri-tert-butyl (5S,12S,16S)-3,6,14-trioxo-1-phenyl-5-[(quinolin-6-yl)methyl]-2-oxa-4,7,13,15-tetraazaoctadecane-12,16,18-tricarboxylate (1.28 g, 1.56 mmol) in ethanol (10 mL) was added palladium (10% on charcoal, 166 mg, 156 μmol) an the mixture was stirred under a hydrogen atmosphere at 50° C. for 5 h. After cooling to r.t. the mixture was filtered and the filtrate was concentrated under reduced pressure. The crude product was purified by preparative HPLC (C18, acetonitrile/1% ammonia) to give the title compound (550 mg, 80% purity, 41% yield).

LC-MS (OA01b02): Rₜ=1.27 min; MS (ESIpos): m/z=687 [M+H]⁺.

Intermediate 304

### tri-tert-butyl (3S,10S,14S)-1-[trans-4-({[(benzyloxy)carbonyl]amino}methyl)cyclohexyl]-1,4,12-trioxo-3-[(quinolin-6-yl)methyl]-2,5,11,13-tetraazahexadecane-10,14,16-tricarboxylate

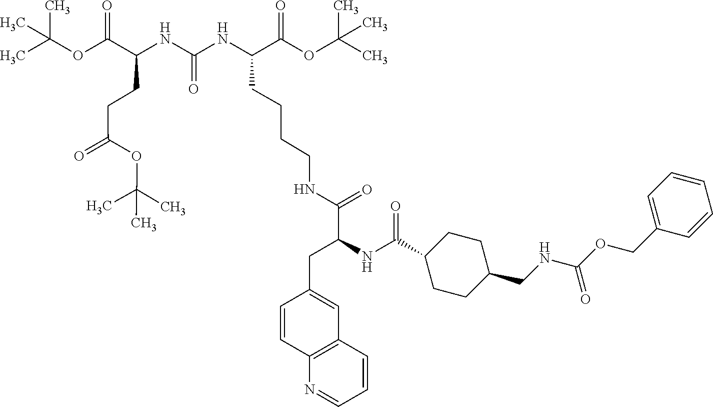 [SMILES: CC(C)(C)OC(=O)CC[C@H](NC(=O)N[C@@H](CCCCNC(=O)[C@H](Cc1ccc2ncccc2c1)NC(=O)[C@H]1CC[C@H](CNC(=O)OCc2ccccc2)CC1)C(=O)OC(C)(C)C)C(=O)OC(C)(C)C]

A mixture of di-tert-butyl (2S)-2-({[(2S)-6-{[(2S)-2-amino-3-(quinolin-6-yl)propanoyl]amino}-1-tert-butoxy-1-oxohexan-2-yl]carbamoyl}amino)pentanedioate (105 mg, 80% purity, 122 μmol), (1r,4r)-4-({[(benzyloxy)carbonyl]amino}methyl)cyclohexane-1-carboxylic acid (39.3 mg, 135 μmol; CAS-RN:[27687-12-3]), HATU (55.9 mg, 147 μmol), 4-methylmorpholine (34 μl, 310 μmol), and DMF (1.9 mL) was stirred at r.t. for 3 h. After that the mixture was separated between DCM and water and the organic phase was filtered through a phase separator and concentrated under reduced pressure. The crude product was purified by preparative HPLC (C18, acetonitrile/1% TFA gradient) to give the title compound (130 mg, 90% purity, 100% yield).

LC-MS (Method 1): Rₜ=1.39 min; MS (ESIpos): m/z=959 [M+H]⁺.

Intermediate 305

### tri-tert-butyl (3S,10S,14S)-1-[trans-4-(aminomethyl)cyclohexyl]-1,4,12-trioxo-3-[(quinolin-6-yl)methyl]-2,5,11,13-tetraazahexadecane-10,14,16-tricarboxylate

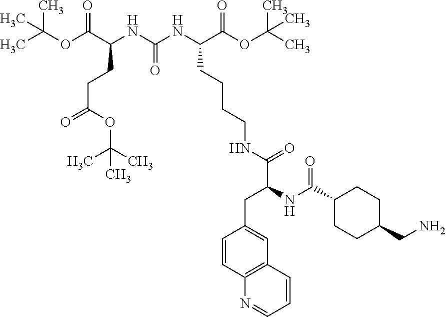 [SMILES: CC(C)(C)OC(=O)CC[C@H](NC(=O)N[C@@H](CCCCNC(=O)[C@H](Cc1ccc2ncccc2c1)NC(=O)[C@H]1CC[C@H](CN)CC1)C(=O)OC(C)(C)C)C(=O)OC(C)(C)C]

To a solution of tri-tert-butyl (3S,10S,14S)-1-[4-({[(benzyloxy)carbonyl]amino}methyl)cyclohexyl]-1,4,12-trioxo-3-[(quinolin-6-yl)methyl]-2,5,11,13-tetraazahexadecane-10,14,16-tricarboxylate (130 mg, 136 μmol) in ethanol (2 mL) was added palladium (10% on charcoal, 14.4 mg, 13.6 μmol) an the mixture was stirred under a hydrogen atmosphere at r.t. for 3 h. After that the mixture was filtered and the filtrate was concentrated under reduced pressure. The crude product was purified by preparative HPLC (C18, acetonitrile/1% ammonia) to give the title compound (14 mg, 95% purity, 12% yield).

LC-MS (Method 2): Rₜ=1.38 min; MS (ESIneg): m/z=824 [M−H]⁻

### Example 48-A

### (3S,10S,14S)-1-[trans-4-(aminomethyl)cyclohexyl]-1,4,12-trioxo-3-[(quinolin-6-yl)methyl]-2,5,11,13-tetraazahexadecane-10,14,16-tricarboxylic acid

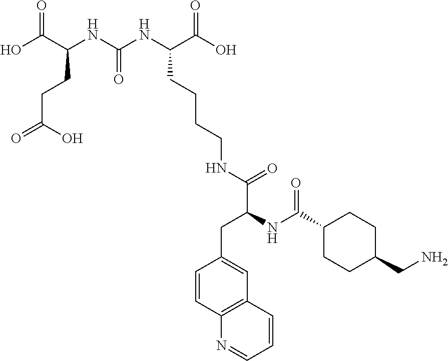 [SMILES: NC[C@H]1CC[C@H](C(=O)N[C@@H](Cc2ccc3ncccc3c2)C(=O)NCCCC[C@H](NC(=O)N[C@@H](CCC(=O)O)C(=O)O)C(=O)O)CC1]

A mixture of tri-tert-butyl (3S,10S,14S)-1-[trans-4-(aminomethyl)cyclohexyl]-1,4,12-trioxo-3-[(quinolin-6-yl)methyl]-2,5,11,13-tetraazahexadecane-10,14,16-tricarboxylate (15.0 mg, 18.2 μmol) and hydrogenchloride (4M in dioxane, 91 μL) was stirred for 14 h at r.t. After that the mixture was concentrated under reduced pressure and the crude product was purified by preparative HPLC (C18, acetonitrile/1% TFA) to give the title compound (5.2 mg, 95% purity, 41% yield).

LC-MS (Method 1): Rₜ=0.48 min; MS (ESIpos): m/z=657 [M+H]⁺

¹H-NMR (400 MHz, DMSO-d6) δ[ppm]: 0.830 (0.46), 0.861 (0.68), 0.890 (0.51), 1.194 (0.54), 1.231 (1.71), 1.248 (1.23), 1.306 (0.78), 1.325 (0.79), 1.458 (0.74), 1.479 (0.63), 1.577 (0.44), 1.638 (0.73), 1.667 (0.64), 1.714 (0.61), 1.735 (0.73), 1.752 (0.61), 2.077 (0.50), 2.214 (0.85), 2.233 (1.41), 2.253 (0.66), 2.322 (0.54), 2.326 (0.70), 2.331 (0.53), 2.518 (4.04), 2.523 (16.00), 2.539 (3.74), 2.589 (1.59), 2.602 (1.34), 2.664 (0.55), 2.669 (0.73), 2.673 (0.55), 2.950 (0.50), 2.959 (0.58), 2.984 (0.66), 3.014 (0.75), 3.128 (0.58), 3.140 (0.65), 3.162 (0.63), 3.174 (0.56), 3.990 (0.51), 4.003 (0.54), 4.051 (0.51), 4.066 (0.51), 4.550 (0.45), 4.564 (0.45), 6.291 (0.58), 6.311 (0.58), 6.326 (0.75), 6.347 (0.68), 7.471 (0.91), 7.481 (0.90), 7.491 (0.85), 7.502 (0.90), 7.634 (0.79), 7.639 (0.84), 7.656 (0.88), 7.660 (0.96), 7.740 (1.46), 7.890 (1.44), 7.912 (1.20), 7.991 (0.46), 8.005 (0.83), 8.019 (0.48), 8.051 (0.83), 8.072 (0.76), 8.233 (0.89), 8.252 (0.81), 8.826 (1.00), 8.831 (1.03), 8.837 (0.99), 8.841 (0.95).

#49 Linker

Intermediate 306

### tri-tert-butyl (3S,10S,14S)-1-{5-[({[(9H-fluoren-9-yl)methoxy]carbonyl}amino)methyl]pyridin-2-yl}-1,4,12-trioxo-3-[(quinolin-6-yl)methyl]-2,5,11,13-tetraazahexadecane-10,14,16-tricarboxylate

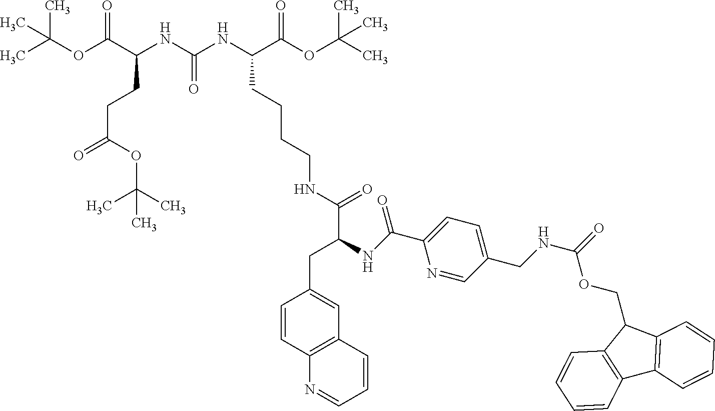 [SMILES: CC(C)(C)OC(=O)CC[C@H](NC(=O)N[C@@H](CCCCNC(=O)[C@H](Cc1ccc2ncccc2c1)NC(=O)c1ccc(CNC(=O)OCC2c3ccccc3-c3ccccc32)cn1)C(=O)OC(C)(C)C)C(=O)OC(C)(C)C]

A mixture of di-tert-butyl (2S)-2-({[(2S)-6-{[(2S)-2-amino-3-(quinolin-6-yl)propanoyl]amino}-1-tert-butoxy-1-oxohexan-2-yl]carbamoyl}amino)pentanedioate (225 mg, 80% purity, 262 μmol), 5-[({[(9H-fluoren-9-yl)methoxy]carbonyl}amino)methyl]pyridine-2-carboxylic acid (98.3 mg, 262 μmol), HATU (120 mg, 315 μmol), 4-methylmorpholine (72 μl, 660 μmol), and DMF (4 mL) was stirred at r.t. for 3 h. After that the mixture was separated between DCM/water and the organic phase was filtered through a phase separator. The filtrate was concentrated under reduced pressure and the residue purified by preparative HPLC (C18, acetonitrile/1% TFA) to give the title compound (175 mg, 91% purity, 58% yield).

LC-MS (Method 1): Rₜ=1.49 min; MS (ESIpos): m/z=1043 [M+H]⁺

Intermediate 307

### tri-tert-butyl (3S,10S,14S)-1-[5-(aminomethyl)pyridin-2-yl]-1,4,12-trioxo-3-[(quinolin-6-yl)methyl]-2,5,11,13-tetraazahexadecane-10,14,16-tricarboxylate

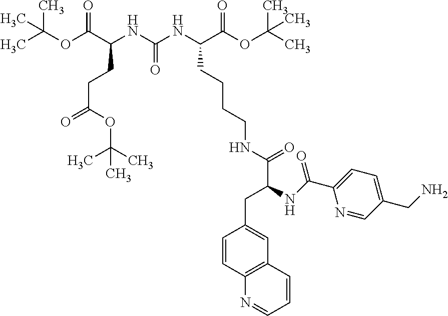 [SMILES: CC(C)(C)OC(=O)CC[C@H](NC(=O)N[C@@H](CCCCNC(=O)[C@H](Cc1ccc2ncccc2c1)NC(=O)c1ccc(CN)cn1)C(=O)OC(C)(C)C)C(=O)OC(C)(C)C]

A mixture of tri-tert-butyl (3S,10S,14S)-1-{5-[({[(9H-fluoren-9-yl)methoxy]carbonyl}amino)methyl]pyridin-2-yl}-1,4,12-trioxo-3-[(quinolin-6-yl)methyl]-2,5,11,13-tetraazahexadecane-10,14,16-tricarboxylate (170 mg, 163 μmol), piperidine (160 μl, 1.6 mmol), and DMF (2.5 mL) was stirred at r.t. for 3 h. After that the mixture was concentrated under reduced pressure and the residue purified by preparative HPLC (C18, acetonitrile/1% ammonia) to give the title compound (65.0 mg, 97% purity, 47% yield).

LC-MS (Method 2): Rₜ=1.27 min; MS (ESIpos): m/z=820.9 [M+H]⁺.

### Example 49-A

### (3S,10S,14S)-1-[5-(aminomethyl)pyridin-2-yl]-1,4,12-trioxo-3-[(quinolin-6-yl)methyl]-2,5,11,13-tetraazahexadecane-10,14,16-tricarboxylic acid

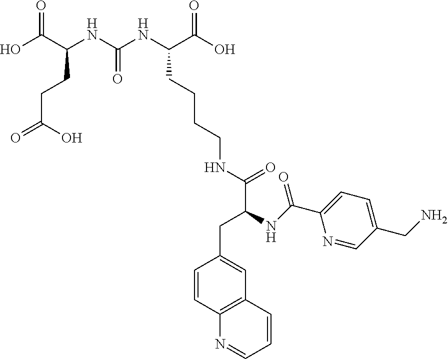 [SMILES: NCc1ccc(C(=O)N[C@@H](Cc2ccc3ncccc3c2)C(=O)NCCCC[C@H](NC(=O)N[C@@H](CCC(=O)O)C(=O)O)C(=O)O)nc1]

A mixture of tri-tert-butyl (3S,10S,14S)-1-[5-(aminomethyl)pyridin-2-yl]-1,4,12-trioxo-3-[(quinolin-6-yl)methyl]-2,5,11,13-tetraazahexadecane-10,14,16-tricarboxylate-(16.0 mg, 19.1 μmol) and hydrogen chloride (4M in dioxane, 96 μL) was stirred at r.t. for 14 h. After that the mixture was concentrated under reduced pressure and the crude product was purified by preparative HPLC (C18, acetonitrile/1% TFA) to give the title compound (3.8 mg, 30% yield).

LC-MS (Method 1): Rₜ=0.50 min; MS (ESIpos): m/z=652 [M+H]⁺

¹H-NMR (400 MHz, DMSO-d6) δ[ppm]: 1.237 (2.92), 1.258 (2.26), 1.333 (2.32), 1.352 (2.92), 1.425 (0.93), 1.444 (1.20), 1.462 (1.00), 1.546 (0.86), 1.559 (1.20), 1.579 (1.00), 1.660 (0.60), 1.674 (1.06), 1.693 (1.39), 1.708 (1.26), 1.727 (1.33), 1.746 (1.53), 1.765 (1.59), 1.781 (1.06), 1.799 (0.73), 2.074 (0.40), 2.137 (0.53), 2.151 (0.73), 2.174 (1.66), 2.194 (1.93), 2.204 (1.86), 2.223 (2.52), 2.244 (1.33), 2.261 (1.00), 2.282 (0.53), 2.327 (4.05), 2.331 (2.92), 2.518 (16.00), 2.523 (10.76), 2.669 (4.18), 2.674 (3.05), 2.835 (0.93), 3.019 (2.52), 3.036 (3.52), 3.052 (3.85), 3.069 (3.45), 3.088 (2.85), 3.103 (2.92), 3.117 (2.72), 3.215 (5.11), 3.249 (7.50), 3.271 (8.43), 3.287 (9.23), 3.300 (10.09), 3.320 (10.36), 3.334 (10.95), 3.385 (12.08), 3.727 (2.06), 3.961 (3.39), 3.971 (3.12), 4.021 (7.97), 4.797 (0.93), 4.818 (1.73), 4.832 (1.73), 4.852 (0.86), 6.154 (1.33), 6.170 (1.26), 6.249 (0.46), 6.269 (0.40), 6.363 (2.46), 6.383 (2.32), 6.650 (1.06), 7.451 (2.79), 7.461 (2.79), 7.471 (2.79), 7.482 (2.79), 7.614 (2.39), 7.619 (2.39), 7.636 (2.66), 7.641 (2.72), 7.759 (4.45), 7.868 (4.25), 7.889 (3.52), 7.920 (1.39), 7.940 (5.84), 7.947 (3.92), 7.973 (0.86), 7.987 (1.26), 8.187 (1.26), 8.201 (2.46), 8.217 (3.39), 8.238 (2.52), 8.515 (0.46), 8.655 (4.12), 8.729 (2.92), 8.750 (2.79), 8.814 (3.32), 8.817 (3.39), 8.824 (3.32), 8.828 (2.99).

### Example 50

### Conjugation of Carboxy-HOPO Chelator to mABs

2,2′,2″,2′″-(propane-1,3-diylbis{nitrilobis[(ethane-2,1-diyl)carbamoyl(3-hydroxy-6-methyl-2-oxopyridine-4,1(2H)-diyl)]})tetraacetic acid (Example A) (24.23 mg) was dissolved in DMA to 30 mg/mL. TSTU (3.4 mg) was dissolved in DMA to 40 mg/mL and added to the carboxy-HOPO solution. 2,4,6-trimethylpyridine (14.8 μL) was added and the solution mixed for 30 min at r.t. The solution was then added to a solution of mAb (50 mg) in PBS (7 mg mAb/mL) droplet by droplet. The mixture was mixed for 30 min at r.t.

Purification using Äkta FPLC (running buffer: 30 mM citrate pH 5.5, flow rate: 1 mL/min, column: HiLoad 10/300 Superdex 200 μg, detection: UV 280/335 nm) of the crude reaction mixture afforded pure mAb-carboxy-HOPO conjugate.

The pure product was analyzed by analytical SEC (running buffer: 10% DMSO in PBS, flow rate: 0.3 mL/min, column: Acuity UPLC Protein BEH SEC, 1.7 μm, 200 Å, 300×4.6 mm, detection: UV 280/335 nm) determining the monomeric purity and CAR. Table 2 list the purity obtained for various mAb-carboxy-HOPO conjugates.

### Example 51: Th-227 Labelling of mAb-Carboxy-HOPO Conjugates

Each mAb-carboxy-HOPO conjugate was dissolved in 30 mM citrate buffer (pH 5.5) containing 50 mg/ml sucrose, 2 mM EDTA and 0.5 mg/mL pABA and 0.075% PS80 was mixed with Th-227 in 0.5M HCl at 2 kBq/μg specific activity at room temperature for 90 min. The labelling efficiently (RCP) was determined by iTLC (2 μL sample was applied on an iTLC strip at 1 cm and the strip developed to 8 cm, then cut at 4 cm and each section counted on a HPGe detector calibrated for Th-227). Table 2 list the RCP obtained for various Th-227 labelled mAb-carboxy-HOPO conjugates.

### Example 52: Zr-89 Labelling of HER2 mAb-Carboxy-HOPO Conjugate

89Zr— was neutralized in 2 M Na₂CO₃. TBS buffer pH 6 and 5 mg/ml gentisic acid were added to a final volume of 1 ml. 50 MBq for each labeling and 250 μg HER2 mAb HOPO chelator conjugate (0.1 mg/ml) was used for each labelling. Labelling samples were incubated at 37° C. for 1 h, then purified by PD10 SEC gravity columns eluting with 10 mM sodium acetate, 240 mM sucrose and 0.02% PS80. Final products were analysed by radio SEC and results are listed in table 1.

TABLE 1
Purity of Zr-89 labelled HER2 chelator conjugates

| mAb chelator | HER2- | HER2-carboxy- |
|---|---|---|
| conjugate | HOPO | HOPO |
| Dimer, UV 280 nm | 3.2% | >1% |
| before labelling | | |
| Dimer, UV 280 nm | 12.1% | 0.9 |
| Dimer, radio | 31.6% | 1.4 |

### Example 5: Determination of Antigen Binding Capacity by IRF of Radiolabelled Antibody Carboxy-HOPO Chelator Conjugates

IRF experiments were performed using PSMA antigen coated magnetic beads. The beads were placed in tubes and diluted to 0.625×10⁶ beads/μl in IRF assay buffer. The diluted beads were divided into two groups: Blocked (BL) and unblocked (UBL). 10 μl naked PSMA antibody was added to the BL group while for the UBL group, just 10 μl of IRF buffer was added. Both groups were incubated for 2 h at r.t. with shaking at 750 rpm. Th-227 labelled antibody carboxy-HOPO chelator conjugates were prepared as described in example 3 and diluted to 5 Bq/μl in IRF assay buffer. The dilutions were confirmed using HPGe detector. At the end of the incubation step, 10 μl of the Th-227 labelled conjugates (˜50 Bq) were added to all the beads (both groups) and incubated for another 2 h at r.t. with shaking at 750 rpm. Then, IRF assay buffer was added to all beads to 1 mL total volume. Tubes filled with samples were placed on a magnet for 2 min. In this way, the samples were divided into 0.5 mL “beads+supernatant” and 0.5 mL “supernatant”. All the samples were counted for 5 min using the HPGe detector and the measured Th-227 activity was used to calculate the % total binding (100×activity on UBL beads/total activity UBL sample), the % nonspecific binding (100×activity on BL beads/total activity BL sample) and the % specific binding (% total binding−% nonspecific binding). IRF results are listed in Table 2 for PSMA antibody carboxy-HOPO chelator conjugates.

TABLE 2
Analytical results for mAb-chelator conjugate

| | Purity by | | | IRF |
|---|---|---|---|---|
| | SEC after | | | Specific |
| mAb-chelator | purification | RCP | | binding |
| conjugate | (% monomer) | (%) | CAR | (%) |
| FAP | 99 | 94 | 0.58 | NA |
| HER2 | 100 | 91 | 0.8 | NA |
| Isotype control | 99 | 96 | 0.83 | NA |
| (IgG2A, non-binding) | | | | |
| PSMA low CAR | 99 | 95 | 0.7 | 78 |
| PSMA high CAR | 98 | 99 | 3 | 79 |
| PSMA-ctrl low CAR | 98 | 100 | 0.8 | 94 |
| PSMA-ctrl high CAR | 99 | 100 | 3 | 77 |

### EXPERIMENTAL SECTION—BIOLOGICAL ASSAYS

Examples were tested in selected biological assays one or more times. When tested more than once, data are reported as either average values or as median values, wherein
the average value, also referred to as the arithmetic mean value, represents the sum of the values obtained divided by the number of times tested, and
the median value represents the middle number of the group of values when ranked in ascending or descending order. If the number of values in the data set is odd, the median is the middle value. If the number of values in the data set is even, the median is the arithmetic mean of the two middle values.

Examples were synthesized one or more times. When synthesized more than once, data from biological assays represent average values or median values calculated utilizing data sets obtained from testing of one or more synthetic batch.

Competition Assay

For binding competition assay 7.5×10\hat{ }5 LNCaP cells were seeded in 500 μl growth medium (RPM11640 (Biochrom #FG1215), 10% FCS Superior (Biochrom #S0615)) in 24 well plates (#3526, Corning). Cells were incubated at 37° C. and 5% CO2 over night. On the following day 400 μl of the media were removed and a total of 200 μl growth medium added supplemented with 3H-PSMA-617 (926.6 GBq/mmol produced in Bayer laboratories) in the presence or absence of competing compounds in triplicates. The final concentration of 3H-PSMA-617 in the test wells was 10 nM, the competing non-radiolabeled PSMA ligands were tested at final concentrations of 10 and 100 nM. After addition of the compounds in growth media the cells were swiveled slightly on a shaker mixing for 10 min followed by incubation for 2 h at 37° C., 5% C02. Following the incubation time the supernatant was removed by pipetting and cells were washed twice with ice cold PBS (total of 800 μl). Cells were lysed in 500 μl of 0.3M NaOH (PanReac AppliChem ITW Reagents) by vigorous shaking for 15 min and the cell lysated pipetted into scintillator tubes (S207-SNAPTWIST® Scintillation Vials 6.5 ml #S207, Simport). In the following the wells were washed with 500 μl PBS and the washing PBS added to the corresponding samples in the sincitallator tubes. To each sample in the scintillator tubes 4 mL of Ultima Gold liquid scintillation cocktail (Sigma-Aldrich #L8286) was added and the samples let stand for at least 30 min. Thereafter radioactivity was counted in a Tri-Carb Liquid Scinzillation Analyzer (Perkin Elmer). Non-specific binding was assessed by measuring remaining binding in the presence of unlabeled PSMA-617 at 5000 nM concentration (500 fold excess) and was found to be below 5%. The percentage of remaining 3H-PSMA-617 bound in the presence of the test compounds compared to total binding of 3H-PSMA-617 alone was calculated for each compound. Result from triplicate values are reported.

By comparing examples 1-B, 1-C monomer, 1-C dimer, 1-C trimer, 1-C tetramer, 1-E, 1-F, 1-PEG and 46-E it can be seen that the compounds -C bearing the HOPO chelator Example A surprisingly show an improved competition against 3H-PSMA-617 on PSMA-expressing LNCaP cells.

By comparing quinoline isomer examples 17-B, 36-B, 44-B, 42-B and 45-B it can be seen that surprisingly the 2-quinolinyl isomer example 17-B shows the best competition amongst all quinoline isomer examples against 3H-PSMA-617 on PSMA-expressing LNCaP cells.

By comparing examples 25-A, 25-B, 26-A, 26-B, 1-A and 1-B it can be seen that surprisingly the pyridine linker examples 25-A, 25-B, 26-A, and 26-B show improved competition in comparison to the cyclohexyl examples 1-A and 1-B against 3H-PSMA-617 on PSMA-expressing LNCaP cells.

By comparing examples 34-A, 34-B3, 1-A, and 1-B3 it can be seen that surprisingly examples 34-A and 34-B bearing the 2-quinolinyl-pyridine linker show an improved competition in comparison to the cyclohexyl examples 1-A and 1-B3 against 3H-PSMA-617 on PSMA-expressing LNCaP cells.

| | % remaining 3H- | % remaining 3H- |
|---|---|---|
| Patent | PSMA-617 | PSMA-617 |
| example | @ 10 nM (mean) | @ 100 nM (mean) |
| 1-B | 66 | 17 |
| 1-A | 90 | 45 |
| 1-B-232Th | 71 | 12 |
| 1-C dimer | 39 | 5 |
| 1-C monomer | 32 | 5 |
| 1-C tetramer | 59 | 7 |
| 1-C trimer | 45 | 9 |
| 1-E | 67 | 21 |
| 1-F | 71 | 21 |
| 1-PEG | 80 | 41 |
| 3-A | 100 | 88 |
| 4-A | 106 | 112 |
| 5-A | 109 | 112 |
| 6-A | 98 | 86 |
| 7-A | 106 | 93 |
| 8-A | 92 | 111 |
| 9-A | 147 | 114 |
| 10-A | 91 | 60 |
| 11-A | 117 | 146 |
| 11a-A | 110 | 103 |
| 12-A | 158 | 128 |
| 13-A | 108 | 124 |
| 14-A | 187 | 129 |
| 15-A | 133 | 111 |
| 16-A | 79 | 90 |
| 17-A | 84 | 56 |
| 17-B | 55 | 12 |
| 17-B-232Th | 46 | 10 |
| 17-C monomer | 42 | 8 |
| 18-A | 78 | 79 |
| 19-A | 84 | 79 |
| 20-A | 88 | 82 |
| 21-A | 70 | 58 |
| 22-A | 113 | 40 |
| 22-C dimer | 30 | 5 |
| 22-C monomer | 47 | 6 |
| 22-C trimer | 33 | 5 |
| 23-A | 59 | 26 |
| 23-B | 66 | 18 |
| 24-A | 66 | 16 |
| 24-B | 59 | 6 |
| 25-A | 93 | 21 |
| 25-A-D | 79 | 67 |
| 25-B | 54 | 5 |
| 26-A | 33 | 6 |
| 26-B | 53 | 6 |
| 27-A | 27 | 7 |
| 27-B | 54 | 6 |
| 28-A | 107 | 60 |
| 28-B | 33 | 90 |
| 29-A | 113 | 92 |
| 30-A | 136 | 96 |
| 31-A | 61 | 78 |
| 32-A | 84 | 46 |
| 32-B | 88 | 35 |
| 33-A | 66 | 72 |
| 33a-B | 74 | 42 |
| 33-B | 114 | 40 |
| 34-A | 50 | 9 |
| 34-B | 43 | 6 |
| 34-C dimer | 42 | 7 |
| 34-C monomer | 58 | 10 |
| 34-C tetramer | 73 | 14 |
| 34-C trimer | 28 | 6 |
| 35-A | 113 | 60 |
| 36-A | 98 | 73 |
| 36-B | 92 | 29 |
| 37-A | 145 | 75 |
| 37-B | 55 | 11 |
| 37-C dimer | 64 | 13 |
| 38-A | 140 | 106 |
| 38-B | 76 | 34 |
| 39-B | 81 | 32 |
| 40-A | 87 | 62 |
| 41-A | 82 | 61 |
| 42-A | 86 | 78 |
| 42-B | 88 | 34 |
| 43-A | 96 | 82 |
| 44-A | 85 | 68 |
| 44-B | 75 | 22 |
| 45-A | 93 | 87 |
| 45-B | 90 | 47 |
| 46-E | 78 | 66 |
| 47-A | 94 | 43 |
| 48-A | 73 | 74 |
| 49-A | 65 | 44 |
| 1-B-D | 80 | 78 |

Cell Proliferation Assay

Inhibition of cell proliferation of Thorium-227 labeled PMSA SMOL ligands was tested different prostate cancer cell lines, namely 22Rv1, VCaP, C4-2 (all from ATCC, Manassas, Va., USA) and LNCaP (DMSZ, Braunschweig). Cells were seeded using the following conditions in 384 well plates (Corning) in 30 μl of growth media at 4000 cells/well for VCaP cells and 1000 cells/well for 22Rv1, C4-2 and LNCaP cells. The growth media for the cell lines were as follows:
22Rv1: RPMI1640 (Biochrom #FG1215),
C4-2: DMEM/Ham's F12 (Biochom #FG4815), 5 μg/ml human recombinant Insulin (Sigma #19278), 0.25 μg/ml Biotin (Sigma #B4639), 25 μg/ml Adenine Hydrochlorid Hydrate (Sigma #A9795), 5 μg/ml Transferrin (Roche #10652202001), 13.6 μg/ml Triiodothyronine (3,3,-5Triodo-L-Thyronine Sodium, Sigma #T6397)
LNCaP: RPMI1640 (Biochrom #FG1215), 10% FCS Superior (Biochrom #S0615)
VCaP: DMEM (high glucose with stable glutamine; Biochrom #FG 0445), 10% FCS Superior (Biochrom #S0615)

Cells were cultivated an incubator at 37° C. and 5% C02 over night. On the following days cells were treated in triplicates with serial dilutions of PSMA-SMOLs labeled with Th-227 at a specific activity of 375 kBq/nmol (RCP>70% as assessed by iTLC analysis; compounds where RCP was between 50-70% in individual experiments are marked with an * in the table) using a D300e Digital Dispenser (Tecan). In sister plates cell viability at treatment start (d=0) was determined using CellTiter-Glo® assay (CTG, Promega, Madison, Wis., USA), by adding 30 μl per well CTG solution, incubation for 10 min and reading of Luminescence in a PheraStarFSX instrument (BMG Labtech). After a 5-day exposure in the presenced of test compounds, cell viability of treated or untreated control cells was determined again using CellTiter-Glo® assay (Promega, Madison, Wis., USA). Dose response curves and calculation of IC50 values (50% inhibition of proliferation) were generated using BELLA-Dose Response Curve (DRC) spreadsheets. The DRC software is a Biobook Spreadsheet that was developed by Bayer AG and Bayer Business Services on the IDBS E-Workbook Suite platform (IDBS: ID Business Solutions Ltd., Guildford, UK). Results show mean IC50 from 1 to 6 independent experiments.

| | Proliferation | Proliferation | Proliferation | Proliferation |
|---|---|---|---|---|
| | assay on cancer | assay on | assay on cancer | assay on |
| | cell lines by | cancer | cell lines by | cancer cell lines |
| | TTCs Default | cell lines | TTCs Default | by TTCs |
| | Pivot #3 | by TTCs | Pivot #3 | Default Pivot #3 |
| | IC50 | Default | IC50 | IC50 |
| | [kBq/mL] | Pivot #3 | [kBq/mL] | [kBq/mL] |
| | VCap | IC50 [kBq/mL] | 22Rv1 | LnCap |
| | 4000cells | C4-2 1000cells | 1000cells | 1000cells |
| | 375kBq/nmol | 375kBq/nmol | 375kBq/nmol | 375kBq/nmol |
| Patent examples | (mean) | (mean) | (mean) | (mean) |
| 1-B-227Th | 0.38 | 0.17 | 0.71 | 0.03 |
| 1-B-D-227Th | 4.52 | 3.15 | 1.28 | 0.49 |
| 1-C dimer-227Th | 1.55 | 1.12 | 1.04 | n.d. |
| 1-C monomer-227Th* | 1.23 | 0.36 | 1.15 | 0.02 |
| 1-C tetramer-227Th | 3.16 | 2.38 | 0.75 | n.d. |
| 1-C trimer-227Th | 3.11 | 2.24 | 0.74 | n.d. |
| 1-F-227Th | 3.07 | 2.14 | 1.06 | n.d. |
| 1-PEG-227Th | 0.64 | 0.44 | n.d. | 0.1 |
| 17-B-227Th | 0.73 | 0.15 | 0.8 | n.d. |
| 17-C monomer-227Th | 1.48 | 0.5 | 0.86 | 0.04 |
| 22-C dimer-227Th | 3.65 | 2.56 | 0.89 | n.d. |
| 22-C monomer-227Th | 2.01 | 0.43 | 0.72 | n.d. |
| 22-C trimer-227Th | 2.62 | 1.82 | 0.84 | n.d. |
| 23-B-227Th* | 3 | 1.32 | 0.96 | n.d. |
| 24-B-227Th* | 0.68 | 0.12 | 0.56 | n.d. |
| 25-B-227Th | 1.51 | 0.1 | 0.73 | n.d. |
| 25-C-D-227Th* | 2.26 | 2.95 | n.d. | 0.49 |
| 26-B-227Th | 1.15 | 0.12 | 0.87 | n.d. |
| 27-B-227Th | 0.73 | 0.08 | 0.65 | n.d. |
| 28-B-227Th | 4.77 | 2.22 | 1.22 | n.d. |
| 32-B-227Th | 1.28 | 4.43 | n.d. | 0.21 |
| 33-B-227Th | 0.58 | 3.78 | n.d. | 0.13 |
| 33a-B-227Th | 1.22 | 4.63 | n.d. | 0.20 |
| 36-B-227Th | 0.75 | 3.88 | n.d. | 0.16 |
| 34-B 227Th | 1.75 | 0.63 | 0.76 | n.d. |
| 34-C monomer 227Th | 0.99 | 0.15 | 1.28 | 0.02 |
| 34-C dimer 227Th | 1.25 | 0.46 | 1.36 | 0.02 |
| 34-C tetramer 227Th | 1.96 | 1.55 | 1.34 | 0.03 |
| 34-C trimer 227Th | 1.1 | 0.28 | 1.25 | 0.03 |
| 37-B-227Th | 0.87 | 1.19 | n.d. | 0.13 |
| 37-C dimer-227Th | 1.53 | 0.48 | 1.06 | n.d. |
| 38-B-227Th | 0.44 | 0.91 | n.d. | 0.10 |
| 42-B-227Th | 0.70 | 0.72 | n.d. | 0.10 |
| 44-B-227Th | 0.71 | 0.67 | n.d. | 0.08 |
| 45-B-227Th | 0.94 | 1.02 | n.d. | 0.14 |
| 46-E 227Th | 1.37 | 0.95 | 0.68 | 0.18 |

*radiochemical purity as detected by iTLC was between 50 and 70% only in individual experiments, whereas it was > 70% in all other experiments

It can be seen that surprisingly the 227Th-labelled compounds of the invention examples 1-C monomer, 1-C dimer, 1-C trimer, 1-C tetramer, 17-C monomer, 22-C monomer, 22-C dimer, 22-C trimer, 34-C monomer, 34-C dimer, 34-C trimer, 34-C tetramer and 37-C dimer bearing the HOPO chelator Example A show high antiproliferative activity on VCap, C4-2, 22Rv1 and LnCap cells. Even more surprisingly the 227Th-labelled compounds of the invention examples 34-C monomer, 34-C dimer, 34-C trimer and 34-C tetramer show an improved antiproliferative activity on VCap and C4-2 cells in comparison to 227Th-labelled compounds examples 1-C monomer, 1-C dimer, 1-C trimer and 1-C tetramer. By comparing examples 1-C-monomer-227Th, 1-PEG-227Th and 46-E 227Th it can be seen that surprisingly example 1-C-monomer-227 bearing the HOPO chelator Example A shows a superior antiproliferative activity compared to 1-PEG-227Th and 46-E 227Th on LnCap cells.

Internalization Assay

For internalization assays 24 well plates (#3526, Corning) were coated with 200 μl Poly-L-Lysine (0.1 mg/ml, #3438-100-01, Fa. Cultrex) per well for 2 h at 37° C. After 2 washes with growth medium, 7.5×10\hat{ }5 LNCaP cells were seeded in 500 μl growth medium (RPMI1640 (Biochrom #FG1215), 10% FCS Superior (Biochrom #S0615)) in the coated wells. Cells were incubated at 37° C. and 5% CO2 over night. On the following day 400 μl of the media were removed and a total of 200 μl growth medium added supplemented with PSMA-SMOLs labeled with Th-227 at 375 kBq/nmol in triplicates. Radiochemical purity of the labeling was analyzed by instant thin layer chromatography and found to be above 70% for all compounds used. The final concentration of radiolabeled SMOLs was in the test wells was 10 nM. Cells were incubated for 30 min or 2 h. After the incubation time supernatants were taken off and cells washed with PBS. Thereafter cells were washed twice with 0.2 ml 50 mM Glycine-HCl buffer (#55097, Sigma Aldrich) and once with PBS and the washing solutions combined in counting vials (#TK75-018, Fa. Corning) and subjected to gamma counting (1470 Wizard automatic gamma counter, Wallac). The resulting counts were attributed to membrane bound radioactivity. Cells were then lysed with 500 μl 0.3N NaOH (#181691.1211, Fa. PanReac AppliChem ITW Reagents) followed by another wash with 500 μl PBS. The NaOH lysate as well as the final wash were combined in vials and subjected to gamma counting as described above. Measured radioactivity was attributed to internalized fraction. Results were calculated as fmol of Th-227 per/10\hat{ }cells in the membrane bound and internalized fractions from triplicate values. To check for specificity of binding/internalization cells were incubated with the radiolabeled compounds in the presence of 100 fold excess of non-radiolabeled compound and observed cell binding/internalization was found to be below 10% of the non-competed value.

It can be seen that the compound 1-C monomer-227Th bearing the HOPO chelator Example A surprisingly shows an increased intracellular radioactivity after 30 min and 2 h on PSMA-expressing LNCaP cells compared to the compound 1-B-227Th bearing a DOTA chelator.

| | Membrane | | Membrane | |
|---|---|---|---|---|
| | bound | Internalized in | bound in | Internalized |
| | in LNCaP | LNCaP | LNCaP | in LNCaP |
| Patent | fmol/10\hat{ }6 | fmol/10\hat{ }6 | fmol/10\hat{ }6 | fmol/10\hat{ }6 |
| example | cells 30 min | cells 30 min | cells 2 h | cells 2 h |
| 1-B-227Th | 237 | 55 | 438 | 189 |
| 1-C monomer- | 105 | 84 | 260 | 291 |
| 227Th | | | | |
| 1-B-D-227Th | 16 | 3 | 30 | 12 |
| 46-E-227Th | 63 | 34 | 234 | 104 |
| 34-C monomer- | 147 | 75 | 359 | 325 |
| 227Th | | | | |
| 34-C dimer- | 79 | 100 | 185 | 299 |
| 227Th | | | | |
| 34-C timer- | 49 | 68 | 93 | 162 |
| 227Th | | | | |
| 34-C tetramer- | 82 | 117 | 183 | 315 |
| 227Th | | | | |

3. Biological In Vivo Experiments:

The LNCaP human prostate cancer model was used for the human xenograft study to evaluate the efficacy or biodistribution of the PSMAsmol compounds.

3.1 Test System, Study Design and Methods

Experiments were initiated after an acclimatization period of at least 7 days. Animals were kept in a 12 hours light/dark cycle, food and water was available ad libitum and housing temperature was 23° C. All animal experiments were conducted in accordance with the German animal welfare law and approved by local authorities.

Study Design Treatment Study

SCID mice were randomly assigned to experimental groups, ten animals per group (efficacy) or n=3-5 per time point of biodistribution study. At the initiation of the study, a testosterone pellet was subcutaneously transplanted, the animals were shaved, tattooed and each cage was labeled with the cage number, study number and number of animals per cage.

Methods and Parameters

LNCaP human prostate cancer cells were cultured as described according to the supplier's protocols. Cells were harvested for transplantation in a subconfluent (70%) state. Animals were injected with 5×10⁶ LNCaP cells suspended in 100% Matrigel subcutaneously into the flank of the mice.

Efficacy study: When tumors reached a predetermined size in mice of 35-50 mm² animals were randomized into treatment and control groups (n=10 animals/group) and treatment with the defined radioactive dose was started. The i.v. application volume was 100 μl/mice.

Tumor response was assessed by determination of the tumor area (product of the longest diameter and its perpendicular) using a caliper. The animal body weight was monitored as a measure for treatment-related toxicity. Measurement of tumor area and body weight was performed two to three times weekly.

Animals were sacrificed when showing signs of toxicity (>20% body weight loss) or when tumors reached a size of approximately 200 mm².

Tumor growth inhibition is presented as T/C ratio (Treatment/Control) calculated with tumor areas when the vehicle control group had to be closed.

Biodistribution study: When tumors reached a predetermined size in mice of ˜50 mm² animals were randomized into groups (n=3 animals/time point) and the radioactive compound was once applied intravenously. The i.v. application volume was 100 μl/mice.

At the predetermined time point, n=3 animals were sacrificed, the organs removed, weight and measured in a Germanium detector to count the Th227 in the sample.

3.2 Results

### Example I

In a study using 1-F-227Th at a dosing of 2×400 kBq/kg weekly followed by 2×750 kBq/kg weekly a T/Cₐᵣₑₐ of 1.03 was reached after 27 days and after an additional treatment with 750 kBq/kg a T/Cₐᵣₑₐ of 0.82 was reached after 37 days. For 34-C monomer 227Th with a single treatment with 750 kBq/kg a T/Cₐᵣₑₐ of 0.37 was already reached after 27 days.

| | T/C [symbol] | T/C [symbol] ( [symbol] | T/C [symbol] ( [symbol] |
|---|---|---|---|
| | ( [symbol] | 27, 2 × | 37, 2 × |
| | 27, | 400 + | 400 + |
| | 1 × 750 | 2 × 750 | 3 × 750 |
| Example | [symbol] /kg) | [symbol] /kg) | [symbol] /kg) |
| [symbol] | 1.00 | 1.00 | 1.00 |
| 34-C | 0.37 | | |
| monomer | | | |
| 227Th | | | |
| 1-F-227Th | | 1.03 | 0.82 |

### Example II

In this study the following treatment groups were included: vehicle control, 1-B-227Th, 1-C monomer-227Th, 1-C dimer-227Th. 1-C monomer-227Th and 1-C dimer-227Th were included as two groups with a doses of 750 kBq/kg resp. 375 kBq/kg each. For 1-B-227Th was only one treatment group with 750 kBq/kg. Vehicle control was citrate buffer only. Treatment for all groups was started at a tumor size of approx. 42-49 mm², at day 14 after tumor cell inoculation. Treatment was weekly for 5 weeks with 750 kBq/kg or 375 kBq/kg and some of the animals were followed until day 131. T/Cₐᵣₑₐ was determined on day 33 after start of therapy. The groups with the compounds 1-B-227Th 750 kBq/kg and 1-C monomer-227Th 750 kBq/kg were continued until day 120, were both groups were terminated. Mean tumor size for 1-B-227Th 750 kBq/kg was measured at 20 mm² and 25 mm² for 1-C monomer-227Th 750 kBq/kg, which was counted as full response for both groups and none of the tumors showed regrowth.

| [symbol] | T/C [symbol] |
|---|---|
| [symbol] | 1.00 |
| 1-B-227Th (750 [symbol] /kg) | 0.18 |
| 1-C monomer-227Th | 0.23 |
| (750 [symbol] /kg) | |
| 1-C monomer-227Th | 0.66 |
| (375 [symbol] /kg) | |
| 1-C dimer-227Th | 0.34 |
| (750 [symbol] /kg) | |
| 1-C dimer-227Th | 0.60 |
| (375 [symbol] /kg) | |

### Example III

In this study the following treatment groups were included: vehicle control, 1-B-227Th, 22-C dimer, 34-B 227Th and 1-C tetramer. Treatment doses for all groups was a single treatment of 1.5 MBq/kg. Vehicle control was 0.9 NaCl only. Treatment for all groups except for group 2 was started at a tumor size of approx. 49 mm², at day 14 after tumor cell inoculation. Group 2 started one week later due to labeling problems at first. T/Cₐᵣₑₐ was determined on day 64 after start of therapy.

| Example | T/C [symbol] |
|---|---|
| [symbol] | 1.00 |
| 1-B-227Th | 0.51 |
| 22-C dimer | 0.19 |
| 34-B 227Th | 0.23 |
| 1-C [symbol] | 0.36 |

### Example IV

In this study the following treatment groups were included: vehicle control, 1-B-D-227Th, 1-C monomer-227Th, 22-C dimer-227Th, 22-C trimer-227Th, 34-B 227Th, 34-C monomer 227Th, 34-C dimer 227Th, 34-C trimer 227Th, 1-C monomer-227Th and 1-C dimer-227Th. Treatment for all groups started at a tumor size of approx. 58 mm², at day 20 after tumor cell inoculation. Treatment was once with 1 MBq/kg and the animals were followed until day 47. T/Cₐᵣₑₐ was determined on day 36 after start of therapy.

| Example | T/C [symbol] |
|---|---|
| [symbol] | 1.00 |
| 1-B-D-227Th | 0.81 |
| 1-C monomer-227Th | 0.33 |
| 22-C dimer-227Th | 0.67 |
| 22-C trimer-227Th | 0.64 |
| 34-B 227Th | 0.66 |
| 34-C monomer 227Th | 0.51 |
| 34-C dimer 227Th | 0.46 |
| 34-C [symbol] 227Th | 0.41 |
| 1-C monomer-227Th | 0.35 |
| 1-C dimer-227Th | 0.48 |

### Example V

In this study the following treatment groups were included: vehicle control, 1-B-227Th, 1-B-D-227Th, 34-C monomer 227Th, 34-C dimer 227Th, 34-C trimer 227Th and 17-C monomer-227Th. Treatment for all groups started at a tumor size of approx. 48 mm², at day 21 after tumor cell inoculation. Treatment was once with 750 kBq/kg. and the animals were followed until day 29. T/Cₐᵣₑₐ was determined on day 27 after start of therapy.

| Example | T/C [symbol] |
|---|---|
| [symbol] | 1.00 |
| 1-B-227Th | 0.29 |
| 1-B-D-227Th | 0.91 |
| 34-C monomer 227Th | 0.37 |
| 34-C dimer 227Th | 0.38 |
| 34-C [symbol] 227Th | 0.56 |
| 17-C monomer-227Th | 0.36 |

### Example VI

In this biodistribution study the compounds 1-F, 1-1B and 1-PEG-227Th were applied with 500 kBq/kg per animal. At the corresponding time point 3-5 animals were sacrificed, organs removed and measured in the Germanium detector. Values below are given at % ID Th-227/g organ.

| 1-F-227Th | 0.25 h | 1 hr | 6 hr | 24 h | 72 h | 168 h | 336 h |
|---|---|---|---|---|---|---|---|
| Blood | 4.80 | 2.28 | 0.35 | 0.06 | 0.02 | 0.00 | 0.00 |
| Tumor | 9.36 | 10.58 | 13.03 | 6.05 | 3.40 | 1.13 | 0.74 |
| Liver | 2.63 | 2.41 | 2.19 | 2.49 | 1.96 | 2.14 | 1.69 |
| Spleen | 2.35 | 1.86 | 1.51 | 0.94 | 1.14 | 1.34 | 1.41 |
| Kidneys | 51.79 | 58.96 | 67.34 | 34.39 | 12.99 | 6.79 | 2.29 |
| Femur and tibia | 1.46 | 1.68 | 1.30 | 0.88 | 0.79 | 1.07 | 1.18 |
| Prostate | 3.62 | 4.83 | 1.97 | 0.34 | 0.68 | 0.13 | 0.22 |
| Salivary gland | 0.31 | 2.68 | 1.48 | 0.75 | 0.73 | 0.74 | 0.42 |

| 1-B-227Th | 0.25 h | 1 hr | 6 hr | 24 h | 72 h | 168 h | 336 h |
|---|---|---|---|---|---|---|---|
| Blood | 1.68 | 0.37 | 0.06 | 0.01 | 0.00 | 0.00 | 0.00 |
| Tumor | 8.41 | 10.87 | 9.80 | 14.45 | 21.94 | 9.80 | 7.78 |
| Liver | 0.92 | 0.41 | 0.19 | 0.18 | 0.26 | 0.21 | 0.17 |
| Spleen | 6.51 | 6.04 | 0.73 | 0.33 | 0.92 | 0.31 | 0.15 |
| Kidneys | 114.94 | 150.81 | 67.34 | 26.81 | 19.30 | 3.56 | 0.78 |
| Femur and tibia | 1.31 | 0.78 | 0.31 | 0.23 | 0.33 | 0.18 | 0.25 |
| Prostate | 8.53 | 9.05 | 6.41 | 0.00 | 0.00 | 0.00 | 0.00 |
| Salivary gland | 10.22 | 17.15 | 2.84 | 1.24 | 0.73 | 0.00 | 0.00 |

| 1-PEG-227Th | 0.25 h | 1 hr | 6 hr | 24 h | 72 h | 168 h | 336 h |
|---|---|---|---|---|---|---|---|
| Blood | 2.95 | 0.44 | 0.10 | 0.04 | 0.01 | 0.00 | 0.00 |
| Tumor | 9.05 | 5.24 | 4.39 | 4.10 | 3.06 | 1.89 | 1.06 |
| Liver | 1.01 | 0.43 | 0.20 | 0.23 | 0.23 | 0.25 | 0.21 |
| Spleen | 1.76 | 1.27 | 0.43 | 0.33 | 0.95 | 0.35 | 0.20 |
| Kidneys | 48.56 | 33.55 | 26.80 | 16.26 | 8.40 | 5.88 | 3.25 |
| Femur and tibia | 1.43 | 1.12 | 0.65 | 0.72 | 4.68 | 0.96 | 0.70 |
| Prostate | 51.66 | 6.57 | 1.96 | 0.00 | 1.45 | 0.00 | 0.00 |
| Salivary gland | 6.53 | 2.48 | 3.30 | 1.84 | 1.10 | 0.00 | 0.12 |

### Example VI

In this biodistribution study the compounds 1-C monomer-227Th and 1-C dimer-227Th were applied with 500 kBq/kg per animal. At the corresponding time point 3 animals were sacrificed, organs removed and measured in the Germanium detector. Values below are given at % ID Th-227/g organ.

| 1-C monamer-Th227 | 1 h | 24 h | 72 h | 168 h | 336 h |
|---|---|---|---|---|---|
| Blood | 0.63 | 0.03 | 0.01 | 0.00 | 0.00 |
| Tumor | 31.73 | 16.36 | 9.02 | 6.20 | 2.78 |
| Liver | 0.69 | 0.35 | 0.36 | 0.33 | 0.31 |
| Spleen | 4.69 | 1.79 | 0.76 | 0.78 | 0.70 |
| Kidneys | 119.59 | 72.59 | 24.80 | 12.66. | 3.41 |
| Salivary glands | 9.62 | 1.41 | 1.13 | 1.23 | 1.29 |
| Femur bone | 0.96 | 1.08 | 1.12 | 1.28 | 1.13 |

| 1-C dimer-Th227 | 1 h | 24 h | 72 h | 168 h | 336 h |
|---|---|---|---|---|---|
| Blood | 0.90 | 0.07 | 0.01 | 0.01 | 0.00 |
| Tumor | 19.99 | 14.49 | 8.99 | 8.68 | 2.85 |
| Liver | 1.49 | 1.34 | 1.02 | 1.28 | 1.06 |
| Spleen | 2.33 | 1.50 | 0.76 | 1.24 | 0.73 |
| Kidneys | 120.74 | 102.45 | 2.10 | 17.42 | 5.40 |
| Salivary glands | 5.02 | 1.39 | 0.77 | 1.17 | 1.20 |
| Femur bone | 1.39 | 1.65 | 1.25 | 1.76 | 1.55 |

### Example VII

In this biodistribution study the compounds 1-B-227Th, 1-B-D-227Th, 220 dimer-227Th, 34-1B-227Th, 1C-Tetramer-227Th were applied with 1500 kBq/kg per animal. At the corresponding time point 3 animals were sacrificed, organs removed and measured in the Germanium detector. Values below are given at % ID Th-227/g organ.

| 1-B-227Th | 0.25 h | 1 hr | 4 hr | 24 h | 96 h |
|---|---|---|---|---|---|
| blood | 6.68% | 3.85% | 1.05% | 0.02% | 0.00% |
| spleen | 11.41% | 9.16% | 6.17% | 1.24% | 2.79% |
| liver | 2.01% | 1.99% | 1.32% | 1.05% | 1.56% |
| kidney | 36.39% | 38.11% | 32.13% | 11.30% | 1.96% |
| lung | 4.42% | 2.88% | 1.55% | 0.50% | 0.55% |
| bone | 2.42% | 5.16% | 7.25% | 9.38% | 10.31% |
| brain | 0.27% | 0.07% | 0.04% | 0.00% | 0.00% |
| muscle | 1.06% | 0.76% | 0.37% | 0.16% | 0.08% |
| tumor | 6.89% | 7.34% | 13.09% | 9.81% | 10.02% |
| small intestine | 1.14% | 0.84% | 0.74% | 0.23% | 0.21% |
| large intestine | 1.08% | 0.57% | 0.76% | 0.12% | 0.12% |
| salivary glands | 2.81% | 1.62% | 0.72% | 0.32% | 0.42% |
| prostate | 7.80% | 1.15% | 1.06% | 0.00% | 0.00% |

| 1-B-D-227Th | 0.25 h | 1 hr | 4 hr | 24 h | 96 h |
|---|---|---|---|---|---|
| blood | 4.97% | 1.04% | 0.20% | 0.00% | 0.00% |
| spleen | 1.91% | 1.09% | 0.34% | 0.23% | 0.29% |
| liver | 1.25% | 0.59% | 0.43% | 0.31% | 0.31% |
| kidney | 10.76% | 4.11% | 2.97% | 0.83% | 0.37% |
| lung | 3.67% | 0.98% | 0.43% | 0.16% | 0.14% |
| bone | 1.70% | 0.99% | 1.68% | 1.29% | 2.88% |
| brain | 0.18% | 0.04% | 0.01% | 0.00% | 0.00% |
| muscle | 0.88% | 0.24% | 0.05% | 0.00% | 0.00% |
| tumor | 2.08% | 1.16% | 1.59% | 0.95% | 1.05% |
| small intestine | 1.21% | 0.26% | 0.17% | 0.08% | 0.06% |
| large intestine | 0.75% | 0.18% | 0.17% | 0.07% | 0.12% |
| salivary glands | 1.61% | 0.52% | 0.30% | 0.09% | 0.13% |
| prostate | 6.26% | 2.87% | 0.00% | 0.00% | 0.00% |

| 22-C | | | | | |
|---|---|---|---|---|---|
| dimer- | | | | | |
| 227Th | 0.25 h | 1 hr | 4 hr | 24 h | 96 h |
| blood | 3.35% | 0.53% | 0.10% | 0.02% | 0.00% |
| spleen | 13.87% | 8.93% | 1.68% | 1.96% | 0.43% |
| liver | 1.79% | 0.41% | 0.60% | 078% | 0.31% |
| kidney | 61.44% | 96.67% | 45.26% | 11.35% | 2.18% |
| lung | 3.56% | 1.30% | 0.38% | 0.25% | 0.16% |
| bone | 2.50% | 1.12% | 0.74% | 0.46% | 0.83% |
| brain | 0.13% | 0.01% | 0.00% | 0.00% | 0.00% |
| muscle | 1.27% | 0.28% | 0.05% | 0.00% | 0.00% |
| tumor | 4.57% | 4.49% | 2.18% | 2.23% | 0.66% |
| small intestine | 1.64% | 0.38% | 0.21% | 0.10% | 0.04% |
| large intestine | 0.87% | 0.23% | 0.29% | 0.21% | 0.04% |
| salivary glands | 2.63% | 0.69% | 0.36% | 0.18% | 0.19% |
| prostate | 5.47% | 1.66% | 0.20% | 0.02% | 0.00% |

| 34-B-227Th | 0.25 h | 1 hr | 4 hr | 24 h | 96 h |
|---|---|---|---|---|---|
| blood | 1.78% | 0.58% | 0.07% | 0.01% | 0.00% |
| spleen | 17.32% | 19.69% | 11.57% | 1.21% | 0.43% |
| liver | 1.69% | 0.88% | 0.55% | 0.23% | 0.19% |
| kidney | 62.53% | 69.22% | 65.52% | 21.02% | 9.71% |
| lung | 4.04% | 2.79% | 1.48% | 0.27% | 0.13% |
| bone | 1.66% | 1.27% | 1.44% | 1.21% | 1.08% |
| brain | 0.12% | 0.02% | 0.00% | 0.00% | 0.00% |
| muscle | 1.02% | 0.54% | 0.41% | 0.00% | 0.00% |
| tumor | 6.33% | 9.15% | 7.00% | 10.56% | 9.97% |
| small intestine | 1.36% | 0.64% | 1.13% | 0.08% | 0.25% |
| large intestine | 0.67% | 0.45% | 0.79% | 0.10% | 1.44% |
| salivary glands | 3.14% | 1.75% | 1.05% | 0.11% | 0.10% |
| prostate | 0.00% | 0.62% | 4.08% | 0.00% | 0.00% |

| 1-C | | | | | |
|---|---|---|---|---|---|
| tetramer- | | | | | |
| 227Th | 0.25 h | 1 hr | 4 hr | 24 h | 96 h |
| blood | 6.34% | 1.30% | 0.05% | 0.02% | 0.00% |
| spleen | 11.00% | 13.21% | 4.31% | 7.54% | 16.20% |
| liver | 14.38% | 21.94% | 8.61% | 10.30% | 16.11% |
| kidney | 16.73% | 25.85% | 13.56% | 15.94% | 4.94% |
| lung | 2.49% | 1.97% | 0.42% | 0.42% | 0.45% |
| bone | 2.24% | 1.97% | 1.16% | 1.30% | 2.44% |
| brain | 0.17% | 0.09% | 0.01% | 0.00% | 0.00% |
| muscle | 0.60% | 0.50% | 0.05% | 0.16% | 0.15% |
| tumor | 1.14% | 3.16% | 0.23% | 3.19% | 1.07% |
| small | 1.14% | 1.12% | 0.56% | 0.62% | 0.57% |
| intestine | | | | | |
| large | 0.91% | 0.92% | 0.33% | 0.42% | 0.55% |
| intestine | | | | | |
| salivary | 4.77% | 3.54% | 1.21% | 1.71% | 1.82% |
| glands | | | | | |
| prostate | 1.26% | 1.71% | 0.00% | 0.00% | 0.00% |

The compound 34-B-227Th showed a good and stable accumulation in the tumor and after a 1.5 MBq/kg treatment had a T/Cₐᵣₑₐ of 0.23 (example Ill) resp. T/Cₐᵣₑₐ of 0.66 (example IV) after 1 MBq/kg treatment. The compound 34-C monomer-227Th and compound 34-C dimer-227Th both showed after 750 kBq/kg treatment T/Cₐᵣₑₐ of 0.37 resp. 0.38 (example V).

### Example VII

Comparing the percent of injected dose per gram tissue between 1-F-Th227, 1-PEG-227Th, 1-C-monomer-227Th and 1-C-Dimer-227Th, 1-F-227Th shows the highest background which can been seen by the highest values in blood, liver and spleen. Adding the PEG-linker as in compound 1-PEG-227Th reduced the overall background of the compound leading to low values in blood, liver and spleen, but also in low tumor accumulation. 1-C-monomer-227Th and 1-C-Dimer-227Th show lower background compared to 1-F-227Th in blood, liver and spleen but much higher accumulation in the tumor.

### Example VIII

Cell Cytotoxicity Assay to Determine the Potency and the Specificity of Radiolabelled Antibody Carboxy-HOPO Chelator Conjugates

Cells were harvested and seeded at 500 cells/wells (30 μl) in 384 well plates and placed in the incubator at 37° C. and 5% C02 overnight. The cells were treated with antibody carboxy-HOPO chelator conjugates radiolabelled with Th-227 at 20 kBq/μg using a half dilution series starting at 50 kBq/mL over 14 points using a TECAN D300e dispenser. The treated cells were incubated at 37° C. with 5% CO₂ for 5 days. Then, the cells were added 30 μl cell titer glow, incubated 10 min and luminescence was measured in the wells using a microplate reader. The % viability was calculated relatively to the luminescence displayed by untreated cells. The specificity ratio was calculated using statistical analysis and by dividing the IC50 of the isotype non-binding control to the IC50 of the corresponding sample. A high specificity ratio indicates specificity. Table 3 list specific ratio for various Th-227 labelled antibody carboxy-HOPO chelator conjugates.

TABLE 3
Cell cytotoxicity results

| | | | Specificity ratio |
|---|---|---|---|
| | | IC50 | (IC50 |
| | mAb-chelator | (kBq/ | isotype/IC50 |
| Cell line | conjugate | ml) | sample) |
| C4-2 | PSMA(low car) | 0.10 | 24 |
| (100- | PSMA ctrl (low car) | 0.17 | 15 |
| 200 000 | PSMA (high car) | 0.13 | 16 |
| mAbs/cell) | PSMA ctrl (high car) | 0.13 | 16 |

### Example IX: Biodistribution of Radiolabelled Antibody Carboxy-HOPO Chelator Conjugates

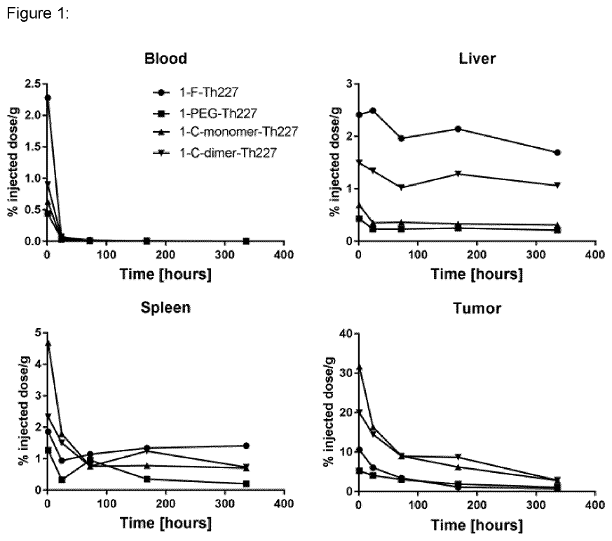

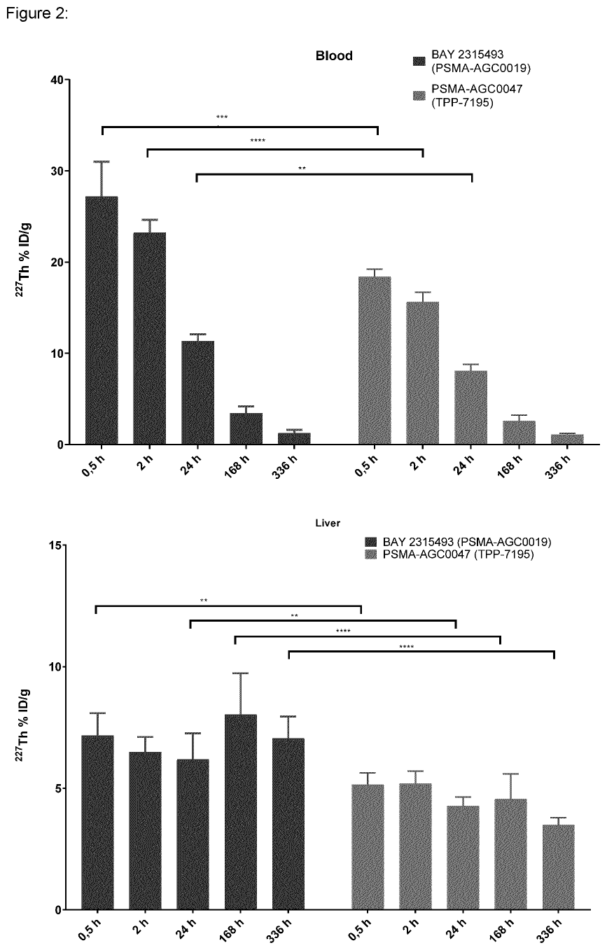

Carboxy-HOPO chelator (designated AGC0047 in FIG. 2) offers altered biodistribution profile for antibody conjugates compared to more hydrophobic HOPO chelates. This is evidenced by the lower liver uptake for this new chelating system and is driven by the increased negative charge (hydrophilicity) of antibody conjugate compared to the antibody conjugated with the 3,2-HOPO (produced according to the methods described in WO2016/096843 and designated AGC0019 in FIG. 2). By altering liver uptake we also anticipate that more thorium-227 will be delivered to the tumor compared to the more hydrophobic HOPO chelates. This is evidenced by the lower liver uptake for this new chelating system and is driven by the increased negative charge (hydrophilicity) of antibody conjugate compared to the antibody conjugated with the 3,2-HOPO. By altering liver uptake we also anticipate that more thorium-227 will be delivered to the target site in the tumor. The results are shown in FIG. 2.

1: A compound of formula (I): 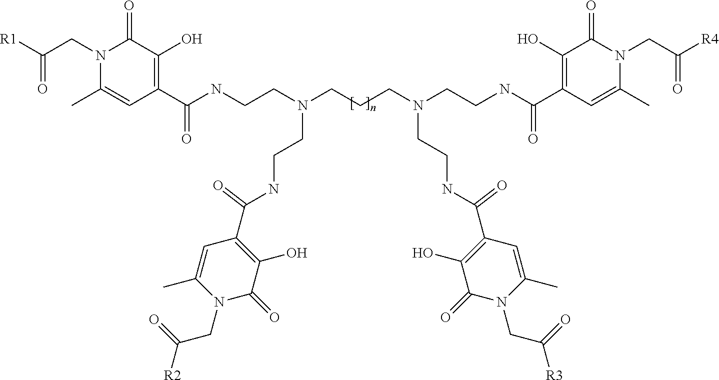
wherein:
n is 1, 2 or 3;
R1, R2, R3 and R4, independently represent OH or Q; and
Q represents a tissue-targeting moiety selected from the group consisting of poly- and oligo-peptides, proteins, DNA and RNA fragments, aptamers polyclonal or monoclonal antibodies, and a mixture of proteins or fragments or constructs of protein;
or a stereoisomer, a hydrate, a solvate, or a salt thereof, or a mixture of same.

2: The compound according to claim 1, 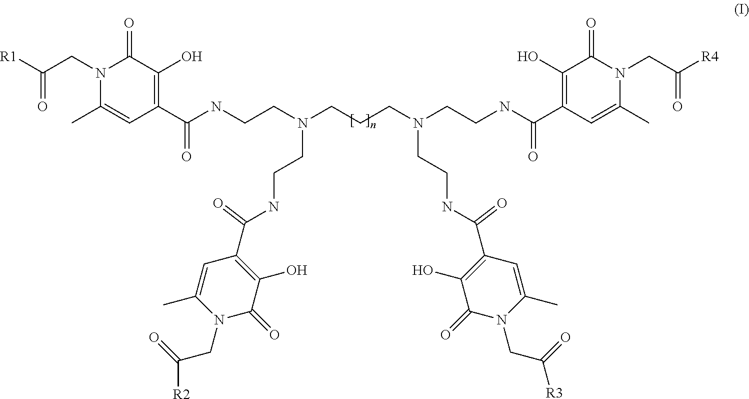
wherein Q represents the following structure: 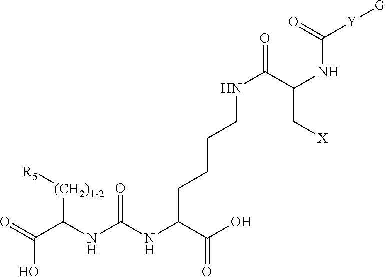
wherein X represents aryl, heteroaryl, butylurea, fluoro-substituted phenyl, butyl-substituted phenyl, quinolinyl and 2-napthyl;
wherein Y represents aryl, heteroaryl, aralkyl, hetaralkyl, C₃-C₈-cycloalkyl, or pyridine;
wherein G represents CH₂N*H or N*H with * being the attachment point to the remainder of compound (I);
and wherein R₅ represents azole, —SO₂H, —SO₃H, —SO₄H, —PO₂H, —PO₃H₂, —PO₃H, —PO₄H₂, —CO₂H, —C(O)R; wherein
R represents —H, —OH, —(C₁-C₁₀)alkyl, —O(C₁-C₁₀)alkyl, —NHR₆, or NR₆R₇;
R₆, R₇ and R₈ each independently represent H, bond, (C₁-C₁₀)alkylene, F, Cl, BR, I, C(O), C(S), —C(S)—NH-benzyl-, —C(O)—NH-benzyl-, —C(O)—(C₁-C₁₀)alkylene, —(CH₂)v-NR₈, —(CH₂)p-NH—C(O)—(CH₂)p-, —(CH₂—CH₂)t-NH—C(O)—(CH₂)p-, —(CH₂)p-COR, —(CH₂)p-C(O)NH—C[(CH₂)p-COR]₃, —C[(CH₂)p-COR]₃, or —(CH₂)p-(C₅-C₁₄)heteroaryl;
wherein v, p and t are independently 0, 1, 2, 3, 4, 5, 6, 7, 8, 9, or 10;
or a stereoisomer, a hydrate, a solvate, or a salt thereof, or a mixture of same.

3: The compound according to claim 1, wherein Q represents a monoclonal antibody with binding affinity for targets selected from the list consisting of FAP, HER2, and PSMA.

4: The compound according to claim 1, wherein compound (I) is radiolabeled with a radionuclide A selected from the group consisting of ⁴³Sc, ⁴⁴Sc, ⁴⁷Sc, ⁶⁴Cu, ⁶⁷Cu, ⁶⁷Ga, ⁶⁸Ga, ⁸⁹Zr, ⁹⁰Y, ¹¹¹In, ¹⁴⁹Tb, ¹⁵²Tb, ¹⁵⁵Tb, ¹⁶¹Tb, ¹⁶⁶Ho, ¹⁷⁷Lu, ¹⁸⁶Re, ¹⁸⁸Re, ²¹²Pb, ²¹²Bi, ²¹³Bi, ²²⁵Ac, ²²⁷Th, and ²³²Th.

5: The compound according to claim 4, wherein the radionuclide A is chelated according to the structure:

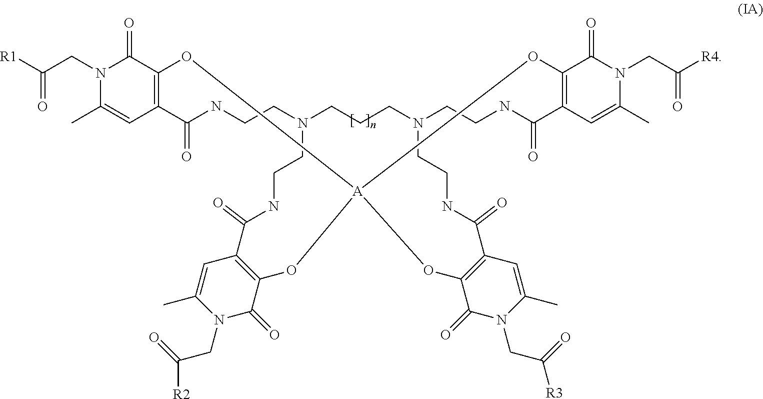

6: The compound according to claim 5, wherein Q represents the following structure: 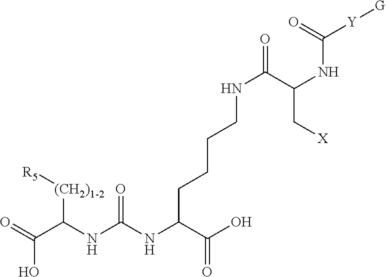
wherein X represents aryl, heteroaryl, butylurea, fluoro-substituted phenyl, butyl-substituted phenyl, quinolinyl and 2-napthyl;
wherein Y represents aryl, heteroaryl, aralkyl, hetaralkyl, C₃-C₈-cycloalkyl, or pyridine;
wherein G represents CH₂N*H or N*H with * being the attachment point to the remainder of compound (I);
and wherein R₅ represents azole, —SO₂H, —SO₃H, —SO₄H, —PO₂H, —PO₃H₂, —PO₃H, —PO₄H₂, —CO₂H, —C(O)R; wherein
R represents —H, —OH, —(C₁-C₁₀)alkyl, —O(C₁-C₁₀)alkyl, —NHR₆, or NR₆R₇;
R₆, R₇ and R₈ each independently represent H, bond, (C₁-C₁₀)alkylene, F, Cl, BR, I, C(O), C(S), —C(S)—NH-benzyl-, —C(O)—NH-benzyl-, —C(O)—(C₁-C₁₀)alkylene, —(CH₂)v-NR₈, —(CH₂)p-NH—C(O)—(CH₂)p-, —(CH₂—CH₂)t-NH—C(O)—(CH₂)p-, —(CH₂)p-COR, —(CH₂)p-C(O)NH—C[(CH₂)p-COR]₃, —C[(CH₂)p-COR]₃, or —(CH₂)p-(C₅-C₁₄)heteroaryl;
wherein v, p and t are independently 0, 1, 2, 3, 4, 5, 6, 7, 8, 9, or 10;
or a stereoisomer, a hydrate, a solvate, or a salt thereof, or a mixture of same.

7: The compound according to claim 4, wherein Q represents a monoclonal antibody with binding affinity for targets selected from the list consisting of FAP, HER2, and PSMA.

8: The compound according to claim 1, wherein:
n is 1;
two of R1, R2, R3 and R4 represent OH and two of R1, R2, R3 and R4 represent Q,
or a stereoisomer, a hydrate, a solvate, or a salt thereof, or a mixture of same.

9: The compound according to claim 1, wherein:
n is 1;
three of R1, R2, R3 and R4 represent OH and one of R1, R2, R3 and R4 represents Q,
or a stereoisomer, a hydrate, a solvate, or a salt thereof, or a mixture of same.

10: The compound according to claim 1, wherein:
n is 1;
one of R1, R2, R3 and R4 represents OH and three of R1, R2, R3 and R4 represent Q,
or a stereoisomer, a hydrate, a solvate, or a salt thereof, or a mixture of same.

11: The compound according to claim 1, wherein:
n is 1;
all of R1, R2, R3 and R4 represent Q,
or a stereoisomer, a hydrate, a solvate, or a salt thereof, or a mixture of same.

12: The compound according to claim 1, wherein:
X represents 2-quinolinyl, and Y represents pyridine,
or a stereoisomer, a hydrate, a solvate, or a salt thereof, or a mixture of same.

13: A method of preparing a compound of formula (I) according to claim 1, comprising reacting an intermediate compound of formula (II): 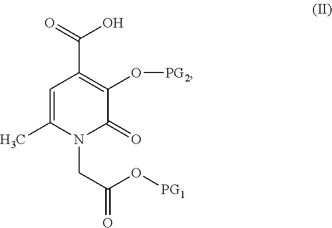
in which PG1 and PG2 represent protecting groups,
with a compound of formula (III): 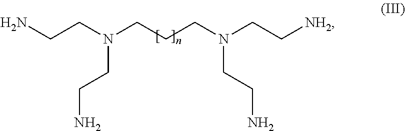
in which n is as defined for the compound of formula (I) according to claim 1,
thereby giving a compound of formula (Ia): 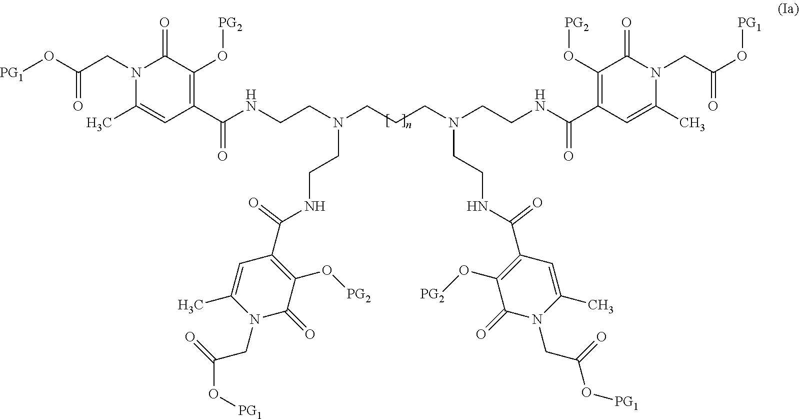
and removing the protecting groups, thereby giving a compound of formula (I) 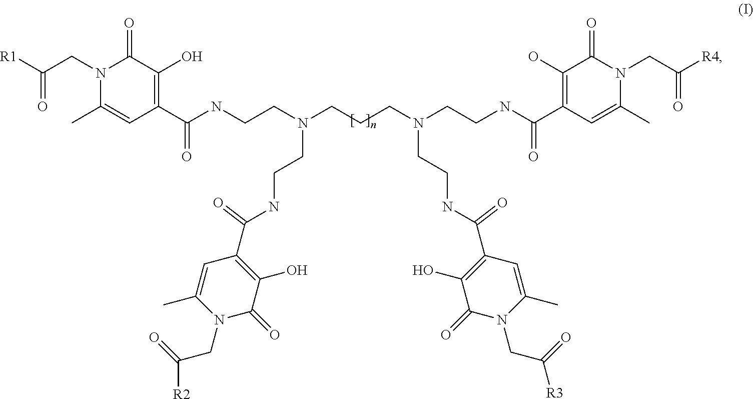
in which R1, R2, R3 and R4 represent OH and n is as defined for the compound of formula (I) according to claim 1.

14. A method for diagnosis, treatment or prophylaxis of a disease in a human or animal in need thereof, comprising administering a compound of formula (I) according to claim 1 to said human or animal.

15: A pharmaceutical composition, comprising a compound of formula (I) according to claim 1, and one or more pharmaceutically acceptable excipients.

16: A pharmaceutical combination comprising:
one or more first active ingredients, wherein the one or more first active ingredients is a compound of formula (I) according to claim 1, and
one or more further active ingredients.

17. (canceled)

18. A method for imaging an internal organ of a mammal in need thereof comprising administering a compound of formula (I) according to claim 1 to said mammal.

19. (canceled)

20: The method of claim 14, wherein the disease is prostate cancer.

21: A method for controlling prostate cancer in a human or animal in need thereof, comprising administering a therapeutically effective amount of at least one compound according to claim 1 to said human or animal.

22: A method for controlling prostate cancer in a human or animal in need thereof, comprising administering a therapeutically effective amount of a pharmaceutical composition according to claim 15 to said human or animal.

23: The pharmaceutical combination of claim 16, wherein the one or more further active agents is one or more cancer agents.
